(19) United States
(12) Patent Application Publication
(10) Pub. No.: US 2026/0256792 A1
(43) Pub. Date: Sep. 3, 2026

(54) TREATMENT OF CHRONIC SPONTANEOUS URTICARIA BY ADMINISTERING (R)-2-[3-[4-AMINO-3-(2-FLUORO-4-PHENOXY-PHENYL)PYRAZOLO[3,4-D]PYRIMIDIN-1-YL]PIPERIDINE-1-CARBONYL]-4-METHYL-4-[4-(OXETAN-3-YL)PIPERAZIN-1-YL]PENT-2-ENENITRILE

(71) Applicant: Principia Biopharma Inc., Morristown, NJ (US)

(72) Inventors: Jessica Gereige, Brookline, MA (US); Leda Mannent, Paris (FR); David Gossage, Beaufort, SC (US)

(73) Assignee: Principia Biopharma Inc., Morristown, NJ (US)

(21) Appl. No.: 19/655,016

(22) Filed: Apr. 22, 2026

Related U.S. Application Data
(63) Continuation of application No. PCT/US2024/052717, filed on Oct. 24, 2024.
(60) Provisional application No. 63/593,171, filed on Oct. 25, 2023.
(60) Provisional application No. 63/652,999, filed on May 29, 2024.

(51) Int. Cl. A61K 31/519; A61P 17/00

(52) U.S. Cl. CPC A61K 31/519; A61P 17/00

(57) ABSTRACT

Methods for treating patients with chronic spontaneous urticaria (CSU) comprising administering at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof are disclosed.

This application is a continuation of International Application No. PCT/US2024/052717, filed Oct. 24, 2024, which claims the benefit of priority to U.S. Application No. 63/652,999 filed May 29, 2024, and U.S. Application No. 63/593,171 filed Oct. 25, 2023, each of which is incorporated herein by reference in its entirety.

Disclosed herein are methods for treating chronic spontaneous urticaria. BTK inhibitors and pharmaceutical compositions comprising the same are also disclosed.

Chronic spontaneous urticaria (CSU), formerly known as chronic idiopathic urticaria (CIU) is a common condition characterized by the spontaneous appearance of pruritic hives and wheals with or without angioedema for more than 6 weeks without a clear inciting factor. CSU has a prevalence between 0.5% and 1% (Maurer et al. 2011). It more commonly affects females than males, and the peak incidence occurs between 20 and 40 years of age. Patients with CSU experience substantial disease burden, including sleep impairment, body image issues, diminished physical and emotional well-being, and impaired performance at school and work (Can et al. 2023; Kolkhir et al. 2022). CSU can thus have significant detrimental impacts on quality of life, productivity, and psychological health (Maurer et al. 2017).

Although the molecular mechanisms are still being elucidated, CSU is thought to be driven by pathogenic activation of mast cells and basophils leading to local release of degranulation products. Of these degranulation products, histamine is felt to play a central role in CSU pathogenesis, driving the pruritus and local vascular permeability (leading to urticaria/angioedema) characteristic of this disease. Of note, emerging data suggest that mast cell and basophil activation in CSU is mediated by activation of the high-affinity IgE receptor, FcεRI, either from development of IgE to autoantigens such as thyroid peroxidase (autoimmune Type I phenotype) or development of activating IgG autoantibodies to IgE or FcεRI (autoimmune Type IIb phenotype) (Bracken et al. 2019; Giménez-Arnau et al. 2021; Kolkhir et al. 2017). Current approved treatments for CSU are targeted against either mast cell degranulation products or the IgE receptor. H1-antihistamines (H1-AH) are first-line therapy in most practice settings. However, approximately 50% of patients fail to achieve symptomatic control with H1-AH at a standard dose. Second-line treatment includes escalation up to four-fold the standard dose of H1-AHs; however, the success rate for patients remains around 45-55%, and dose escalation may be associated with increased somnolence compared to standard dosing of H1-AHs (Armstrong et al. 2023; Kaplan et al. 2023; Kolkhir et al. 2022; Xiao et al. 2023). Omalizumab, an anti-IgE monoclonal antibody which reduces both serum IgE and cell-surface FcεRI, has emerged as an effective treatment for CSU with inadequate response to H1-AH. However, it has been reported that nearly 50% of patients who receive omalizumab have an inadequate response to this therapy (Metz et al. 2017). In particular, patients with Type IIb autoimmunity are less likely to respond to treatment with H1-AH or omalizumab (Marcelino et al. 2021). As such, there is a need for additional treatments for CSU.

Emerging evidence suggests that Bruton's tyrosine kinase (BTK), a non-receptor-tyrosine kinase and a member of the TEC family of kinases, plays a role in signal amplification after FcεRI cross-linking, leading to enhanced activation of mast cells and basophils. Consistent with this observation, BTK antagonists have demonstrated an inhibitory effect on basophil activation by allergens or anti-IgE antibodies. Given its importance in both the generation of antibodies and signaling through FcεRI, BTK has great potential as a target in CSU therapy, as evidenced by multiple clinical studies investigating the effect of BTK inhibitors on patients with CSU who are refractory to treatment with H1-AH, and are omalizumab-naïve.

Compound (I) is a BTK inhibitor of the following structure:

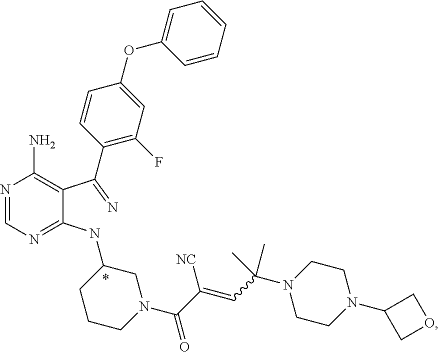 [SMILES: CC(C)(C=C(C#N)C(=O)N1CCCC(Nc2ncnc(N)c2C(=N)c2ccc(Oc3ccccc3)cc2F)C1)N1CCN(C2COC2)CC1]

wherein *C is a stereochemical center. See PCT Publication No. WO 2014/039899, which is incorporated herein by reference, e.g., Example 31.
(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, having the following structure:

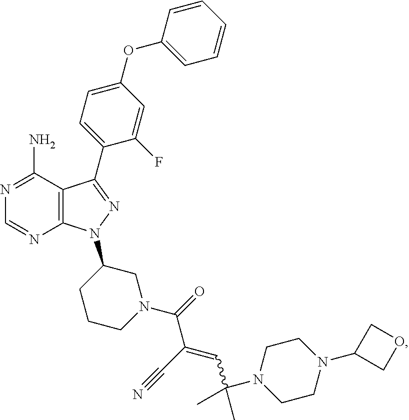 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

is also known as rilzabrutinib. This compound has been disclosed in several patent publications, such as, e.g., PCT Publication Nos. WO 2014/039899, WO 2015/127310, WO 2016/100914, WO 2016/105531, WO 2018/005849, and WO 2021/150723, the contents of each of which are incorporated by reference herein.

Rilzabrutinib is a novel, highly selective, and potent small molecule inhibitor of non-T cell white blood cell signaling via B-cell receptor, FcγR, and/or FcεR signaling of the BTK pathway. In the context of ITP, rilzabrutinib has the potential to (1) inhibit B cell activation and (2) interrupt antibody-coated cell phagocytosis by FCγR in the spleen and liver (Bradshaw et al. 2021, Langrish et al. 2021, Owens et al. 2022).

Rilzabrutinib functions as a reversible covalent BTK inhibitor and is capable of both non-covalently and covalently binding to its target; in particular, its reversible cysteine binding enables high selectivity and precise BTK inhibition without a permanent modification of proteins and peptides (Langrish et al. 2021, Owens et al. 2022, Smith μF et al. 2017). Taken together, these properties allow for enhanced selectivity and extended inhibition with low systemic exposure. In comparison to first and second generation BTKi, rilzabrutinib has shown minimal cross-reactivity with other molecules and is low risk for off-target effects (Smith μF et al. 2017). Importantly, rilzabrutinib's reversible binding minimizes the likelihood of permanently modified peptides (Serafimova IM 2012). In addition, rilzabrutinib shows improved kinase selectivity relative to the covalent BTK inhibitor ibrutinib. Preclinical studies in a broad kinase enzyme inhibition panel showed that 1 μM rilzabrutinib achieved>90% inhibition of just 6 of 251 kinases sharing a common cysteine in their active site. By contrast, 1 μM ibrutinib inhibited 21 kinases. Rilzabrutinib's IC50 values were 1.3 nM for BTK, 0.8 nM for tyrosine protein kinase TEC, 1.0 nM for bone marrow tyrosine kinase on chromosome X (BMX), 1.2 nM for receptor-like kinase (RLK), 6.3 nM for B cell lymphocyte kinase (BLK), and 11 nM for ERBB4. Further preclinical assays with rilzabrutinib showed that binding to BTK persisted while that for other TEC family members decayed rapidly over time.

Rilzabrutinib has shown encouraging results for the treatment of immune-mediated diseases. In humans, rilzabrutinib is rapidly absorbed following oral administration, with a fast half-life (3-4 h) and variable pharmacokinetics (Smith μF et al., 2017).

In Phase 1 studies of rilzabrutinib with 114 healthy volunteers, target BTK occupancy levels were safely and consistently exceeded, suggesting rilzabrutinib may be highly effective in treating autoimmune diseases. Moreover, preclinical and clinical pharmacokinetic and pharmacodynamic data showed that treatment effects endured even after the compound was cleared from circulation, consistent with an extended target residence time (Hill R et al., 2015) and high target occupancy rate (>90% within four hours and high sustained occupancy over 24 h) (Smith μF et al., 2015).

Rilzabrutinib has also demonstrated a favorable safety profile in clinical studies. In contrast with non-selective, irreversible BTK inhibitors, rilzabrutinib does not alter platelet aggregation in healthy volunteers or patients with ITP and thus does not lead to bleeding problems (Langrish et al. 2021, von Hundelshausen and Seiss 2021). As an additional point of contrast with irreversible BTK inhibitors, rilzabrutinib treatment does not exert clinically relevant effects on cardiac repolarization (electrocardiogram parameters including corrected QT interval) in healthy volunteers (N=51), even when administered at supratherapeutic doses (Lipsky and Lamanna 2020). Indeed, the most commonly reported adverse events in healthy volunteers were gastrointestinal adverse events, including nausea/vomiting and diarrhea. No serious adverse events or deaths were reported, and no participants discontinued treatment due to an adverse event (Smith μF 2017). Moreover, based on preclinical reproductive toxicity studies, rilzabrutinib is not expected to harm fetal development or male fertility.

The efficacy of rilzabrutinib has been evaluated in both nonclinical and clinical studies. Rilzabrutinib demonstrated blockade of the rat Arthus reaction, full disease reversal of a rat collagen-induced arthritis model, and reduction in platelet loss in a mouse model of immune thrombocytopenia. The therapeutic effects of rilzabrutinib in pemphigus was studied in dogs with newly diagnosed, naturally occurring pemphigus foliaceus (PF). The drug safely controlled disease without the need for corticosteroid (CS) in 4 of 4 cases. The clinical efficacy of rilzabrutinib was further demonstrated in an open-label, Phase 1/2 study in immune thrombocytopenic purpura (ITP). In this trial, 50% of participants achieved the primary response endpoint after treatment with rilzabrutinib for at least 12 weeks. Furthermore, there were no treatment-emergent serious adverse events (TESAEs) observed. Collectively, these and other studies have shown rilzabrutinib to be a safe and effective inhibitor of BTK.

There is preliminary evidence to support the role of BTK inhibition in patients with CSU, including in patients with CSU who are both refractory to treatment with H1-AH and are omalizumab-naïve (Maurer et al. 2021; Metz et al. 2020). Notably, results from studies on the BTK inhibitor fenebrutinib suggest a differential response based on basophil histamine release assay (BHRA) status, wherein BHRA-positive patients may be more sensitive to BTK inhibition, especially at lower doses, compared to BHRA-negative patients (Metz et al. 2020). Separately, studies suggest that CSU patients who are BHRA-positive are more likely to have a poorer response to omalizumab compared to those who are BHRA-negative. As patients with prior omalizumab use, including omalizumab-incomplete responders may be enriched for BHRA-positive CSU patients, who are in turn more likely to have Type IIb autoimmunity and be less responsive to available treatments, they may be best poised to benefit from BTK inhibition. Patients with prior omalizumab use, including omalizumab-incomplete responders who are BHRA-negative may also stand to benefit from BTK inhibition, although this benefit may be seen at higher doses compared to BHRA-positive patients (Gericke et al. 2016). Accordingly, a dose-ranging Phase 2 study was initiated to assess the efficacy and safety of rilzabrutinib in patients with moderate-to-severe CSU.

Accordingly, disclosed herein are methods of treating chronic spontaneous urticaria (CSU) in a human patient in need thereof, comprising administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.

Further disclosed herein are methods of treating chronic spontaneous urticaria (CSU) in a human patient in need thereof, comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl- 4-[4-(oxetan-3-yl)piperazin-1-yl]pent-2-enenitrile (rilzabrutinib) and pharmaceutically acceptable salts thereof, wherein the human patient in need thereof has CSU refractory to H1 antihistamines (H1-AH) treatment.

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof.

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof.

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof.

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for the manufacture of a medicament for treating chronic spontaneous urticaria (CSU) in a human patient in need thereof.

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof, wherein the human patient in need thereof has CSU refractory to H1 antihistamines (H1-AH) treatment.

Further disclosed herein is a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof, wherein the human patient in need thereof has CSU refractory to H1 antihistamines (H1-AH) treatment.

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof, wherein the human patient in need thereof has CSU refractory to H1 antihistamines (H1-AH) treatment.

Further disclosed herein is the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, in the manufacture of a medicament for treating chronic spontaneous urticaria (CSU) in a human patient in need thereof, wherein the human patient in need thereof has CSU refractory to H1 antihistamines (H1-AH) treatment.

### BRIEF DESCRIPTION OF DRAWINGS

FIG. 1A shows the graphical study design.

FIG. 1B shows patient disposition up to Week 12.

FIG. 2 shows a waterfall plot of UAS7 at Week 12 by treatment group (omalizumab-naïve population). Circles and triangles indicate scores for individual patients at baseline and Week 12, respectively.

FIG. 3 shows a waterfall plot of UAS7 at Week 12 by treatment group (patients with prior omalizumab use, including omalizumab-incomplete responders in ITT population). Circles and triangles indicate scores for individual patients at baseline and Week 12, respectively.

FIG. 4 shows the cumulative probability function of change from baseline in UAS7 at Week 12 (omalizumab-naïve population).

FIG. 5 shows a plot of LS mean change from baseline in UAS7 over time up to Week 12 (ITT population).

FIG. 6 shows a plot of LS mean change from baseline in UAS7 over time up to Week 12 (omalizumab-naïve population).

FIG. 7 shows a plot of LS mean change from baseline in UAS7 over time up to Week 12 (omalizumab-naïve population with outlier removed from analysis).

FIG. 8 shows a plot of mean change from baseline in UAS7 over time up to Week 12 (patients with prior omalizumab use, including omalizumab-incomplete responders in ITT population).

FIG. 9 shows a plot of the proportion of participants with UAS7<=6 over time up to Week 12 (ITT population).

FIG. 10 shows a plot of the proportion of participants with UAS7<=6 over time up to Week 12 (omalizumab-naïve population).

FIG. 11 shows a plot of the proportion of participants with UAS7=0 over time up to Week 12 (ITT population).

FIG. 12 shows a plot of the proportion of participants with UAS7=0 over time up to Week 12 (omalizumab-naïve population).

FIG. 13 shows a plot of the proportion of participants with UAS7 reduction from baseline of 10 points or more over time up to Week 12 (ITT population).

FIG. 14 shows a plot of the proportion of participants with UAS7 reduction from baseline of 10 points or more over time up to Week 12 (omalizumab-naïve population).

FIG. 15 shows a cumulative probability function of change from baseline in ISS7 at Week 12 (omalizumab-naïve population).

FIG. 16 shows a waterfall plot of ISS7 at Week 12 by treatment group (omalizumab-naïve population). Circles and triangles indicate scores for individual patients at baseline and Week 12, respectively.

FIG. 17 shows a waterfall plot of ISS7 at Week 12 by treatment group (patients with prior omalizumab use, including omalizumab-incomplete responders in the ITT population). Circles and triangles indicate scores for individual patients at baseline and Week 12, respectively.

FIG. 18 shows a plot of LS mean change from baseline in ISS7 over time up to Week 12 (ITT population).

FIG. 19 shows a plot of LS mean change from baseline in ISS7 over time up to Week 12 (omalizumab-naïve population).

FIG. 20 shows a plot of LS mean change from baseline in ISS7 over time up to Week 12 (omalizumab-naïve population with outlier removed from analysis).

FIG. 21 shows a plot of mean change from baseline in ISS7 over time up to Week 12 (patients with prior omalizumab use, including omalizumab-incomplete responders in the ITT population).

FIG. 22 shows a cumulative probability function of change from baseline in HSS7 at Week 12 (omalizumab-naïve population).

FIG. 23 shows a waterfall plot of HSS7 at Week 12 by treatment group (omalizumab-naïve population). Circles and triangles indicate scores for individual patients at baseline and Week 12, respectively.

FIG. 24 shows a waterfall plot of HSS7 at Week 12 by treatment group (patients with prior omalizumab use, including omalizumab-incomplete responders in the ITT population). Circles and triangles indicate scores for individual patients at baseline and Week 12, respectively.

FIG. 25 shows a plot of LS mean change from baseline in HSS7 over time up to Week 12 (ITT population).

FIG. 26 shows a plot of LS mean change from baseline in HSS7 over time up to Week 12 (omalizumab-naïve population).

FIG. 27 shows a plot of LS mean change from baseline in HSS7 over time up to Week 12 (omalizumab-naïve population with outlier removed from analysis).

FIG. 28 shows a plot of mean change from baseline in HSS7 over time up to Week 12 (patients with prior omalizumab use, including omalizumab-incomplete responders in the ITT population).

FIG. 29 shows a plot of LS mean change from baseline in AAS7 over time up to Week 12 in participants with angioedema at baseline (ITT population).

FIG. 30 shows a plot of LS mean change from baseline in AAS7 over time up to Week 12 in participants with angioedema at baseline (omalizumab-naïve population).

FIG. 31 shows a cumulative probability function of change from baseline in AAS7 at Week 12 in participants with angioedema at baseline (omalizumab-naïve population).

FIG. 32 shows a waterfall plot of AAS7 at Week 12 by treatment group in participants with angioedema at baseline (omalizumab-naïve population). Circles and triangles indicate scores for individual patients at baseline and Week 12, respectively.

FIG. 33 shows a plot of the proportion of participants with AAS7=0 over time up to Week 12 (participants with angioedema at baseline in the omalizumab-naïve population).

FIG. 34 shows a plot of proportion of participants with AAS7=0 over time up to Week 12 (omalizumab-naïve population with outlier removed from the analysis).

FIG. 35 shows a plot of LS mean change from baseline in UCT over time up to Week 12 (ITT population).

FIG. 36 shows a plot of LS mean change from baseline in UCT over time up to Week 12 (omalizumab-naïve population).

FIG. 37 shows a plot of proportion of participants with UCT≥12 over time up to Week 12 (omalizumab-naïve population).

FIG. 38 shows a plot of LS mean change from baseline in PGIS over time up to Week 12 (omalizumab-naïve population).

FIG. 39 shows a plot of LS mean change from baseline in PGIC over time up to Week 12 (omalizumab-naïve population).

FIG. 40A-E shows a plot of the median percentage change from baseline (with interquartile range) for (A) sMRGPRX2, (B) IL-31, (C) IgG anti-TPO, (D) IgG anti-FcεRI, and (E) IgG anti-IgE.

FIG. 41 shows a plot of the median percentage change from baseline (with interquartile range) for eosinophils.

### DEFINITIONS

Unless otherwise stated, the following terms used in the specification and claims are defined for the purposes of this Application and have the following meanings. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. With regard to specific values, it should be understood that specific values described herein for subject populations (e.g., the subject of the described clinical trial) represent median, mean, or statistical numbers, unless otherwise provided. Accordingly, aspects of the present disclosure requiring a particular value in a subject are supported herein by population data in which the relevant value is assessed to be a meaningful delimitation on the subject population.

As used herein, the term “active pharmaceutical ingredient” or “therapeutic agent” (“API”) refers to a biologically active compound.

As used herein, the term “approved treatment” refers to a medication that has received regulatory authorization, in any country, for its intended use.

As used herein, the terms “administer,” “administering,” or “administration” herein refer to providing, giving, dosing, and/or prescribing by either a health practitioner or an authorized agent and/or putting into, taking, or consuming by the patient or person himself or herself. For example, “administration” of an API to a patient refers to any route (e.g., oral delivery) of introducing or delivering the API to the patient. Administration includes self-administration and administration by another.

As used herein, the term “baseline” refers to a measurement or an average of measurements taken on or before day 1 (D1) of initiating rilzabrutinib treatment. By way of non-limiting example, baseline can be determined for, e.g., UAS7, ISS7, UCT, and AAS7.

As used herein, “BID” and “bid” are used interchangeably to refer to twice a day.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, an amount expressed in terms of “mg of [X]” refers to the total amount in milligrams of [X], i.e., the free base. In some embodiments, rilzabrutinib may be administered as a pharmaceutically acceptable salt of rilzabrutinib, in which case an amount expressed in terms of “mg of rilzabrutinib” refers to the total amount in milligrams of rilzabrutinib, i.e., the free base, plus the equivalent amount of one or more pharmaceutically acceptable salts of rilzabrutinib based on the weight of free base therein. For example, “400 mg of at least one compound chosen from rilzabrutinib and pharmaceutically acceptable salts thereof” includes 400 mg of rilzabrutinib and a concentration of one or more pharmaceutically acceptable salts of rilzabrutinib equivalent to 400 mg of rilzabrutinib.

As used herein, “moderate-to-severe” CSU encompasses both patients with moderate CSU and patients with severe CSU.

As used herein, the terms “prior use of omalizumab” “omalizumab-incomplete responder,” “omalizumab-incomplete responder,” “incomplete responder,” and “incomplete responders” refer to participants who were previously treated with omalizumab and/or exhibited inadequate control of CSU symptoms despite treatment with omalizumab as prescribed.

As used herein, a “pharmaceutically acceptable carrier or excipient” means a carrier or an excipient that is useful in preparing a pharmaceutical composition that is generally safe, and neither biologically nor otherwise undesirable, such as, e.g., a carrier or an excipient that is acceptable for mammalian pharmaceutical use.

As used herein, the term “pharmaceutically acceptable salt” refers to a salt form, e.g., an acid addition salt, of an active pharmaceutical agent that is pharmaceutically acceptable and that possesses the desired pharmacological activity of the API of which the salt is made. Pharmaceutically acceptable salts are well known in the art and include those derived from suitable inorganic and organic acids. Such salts include, but are not limited to, salts formed with inorganic acids such as hydrochloric acid, hydrobromic acid, sulfuric acid, phosphoric acid, and the like; or formed with organic acids such as formic acid, acetic acid, propionic acid, hexanoic acid, lactic acid, malonic acid, succinic acid, malic acid, maleic acid, fumaric acid, tartaric acid, citric acid, benzoic acid, mandelic acid, methanesulfonic acid, ethanesulfonic acid, 1,2-ethanedisulfonic acid, benzenesulfonic acid, 4-toluenesulfonic acid, and the like. S. M. Berge et al. describes pharmaceutically acceptable salts in detail in J. Pharmaceutical Sciences, 1977, 66, 1-19.

As used herein, the terms “rilzabrutinib,” “(R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile;” “the compound of Formula (I);” and “2-[(3R)-3-[4-amino-3-(2-fluoro-4-phenoxy-phenyl)pyrazolo[3,4-d]-pyrimidin-1-yl]piperidine-1-carbonyl]-4-methyl-4-[4-(oxetan-3-yl)piperazin-1-yl]pent-2-enenitrile” are used interchangeably to refer to a compound having the structure:

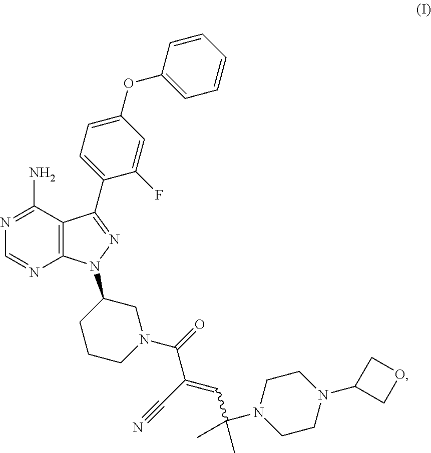 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

which is also referred to as 2-[(3R)-2-[4-amino-3-(2-fluoro-4-phenoxy-phenyl)pyrazolo[3,4-d]pyrimidin-1-yl]piperdine-1-carbonyl]-4-methyl-4[4-(oxetan-3-yl)piperazin-1-yl]-(E and Z)-pent-2-enenitrile; (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile; 1-piperidinepropanenitrile, 3-[4-amino-3-(2-fluoro-4-phenoxyphenyl)-1H-pyrazolo[3,4-d]pyrimidin-1-yl]-a-[2-methyl-2-[4-(3-oxetanyl)-1-piperazinyl]propylidene]-3-oxo-, (3R)-; (EZ)-2-[(3R)-3-[4-amino-3-(2-fluoro-4-phenoxyphenyl)pyrazolo[3,4-d]pyrimidin-1-yl]piperidine-1-carbonyl]-4-methyl-4[4-(oxetan-3-yl)piperazin-1-yl]pent-2-enenitrile; and also by the International Nonproprietary Names for Pharmaceutical Substances (INN) as published by the World Health Organization (https://cdn.who.int/media/docs/default-source/international-nonproprietary-names-(inn)/pl121.pdf?sfvrsn=69617906_15&download=true) having the following structure:

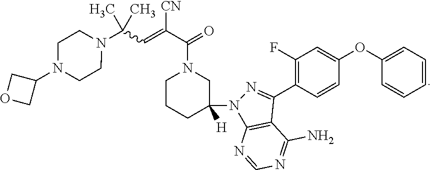 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1 ; "."]

The compound of Formula (I) includes E and Z isomers, as indicated by the wavy bond in the structure shown above. The compound of Formula (I) may be present as a salt form.

A dose of the (E) isomer of rilzabrutinib may contain the corresponding (Z) isomer as an impurity in less than about 1% by weight; a dose of the (Z) isomer of rilzabrutinib may contain the corresponding (E) isomer as an impurity in less than about 1% by weight. When rilzabrutinib is denoted as a mixture of (E) and (Z) isomers of (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, it means that the amount of (E) or (Z) isomer in the mixture is greater than about 1% by weight. In some embodiments, the molar ratio of (E) to (Z) isomer is 9:1. rilzabrutinib or a pharmaceutically acceptable salt thereof may also be referred to herein as a “drug,” “active agent,” “a therapeutically active agent,” or “API.”

As used herein, “QD” and “qd” are used interchangeably to refer to once a day.

As used herein, the terms “refractory” or “refractory to H1 antihistamines” encompasses patients diagnosed with CSU who continue to exhibit symptoms despite treatment with up to 4 times the recommended dose of H1-AH, for example 2 times the recommended dose, 2.5 times the recommended dose, 3 times the recommended dose, 3.5 times the recommended dose, or 4 times the recommended dose.

As used herein, the term “therapeutically effective amount” refers to that of a compound that produces the desired effect for which it is administered (e.g., improvement in CSU or a symptom of CSU, or lessening the severity of CSU or a symptom of CSU). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, “TID” and “tid” are used interchangeably to refer to three times a day.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

The present disclosure provides methods of treating chronic spontaneous urticaria (CSU) in a human patient in need thereof. In some embodiments, the methods comprise administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof. In some embodiments, the methods comprise administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has CSU refractory to H1 antihistamines (H1-AH) treatment. In at least one embodiment, rilzabrutinib is administered to the human patient.

In some embodiments, the human patient has moderate-to-severe CSU. In some embodiments, the human patient has moderate CSU. In some embodiments, the human patient has severe CSU. In some embodiments, the human patient has CSU refractory to H1 antihistamine (H1-AH) treatment.

In some embodiments, the human patient has Type I autoimmune CSU. In some embodiments, the human patient has Type IIb autoimmune CSU.

In some embodiments, the human patient has angioedema. In some embodiments, the human patient does not have angioedema.

In some embodiments, the human patient achieves a Urticaria Activity Score (UAS7) that is lower than baseline UAS7. In some embodiments, the human patient achieves a UAS7 that is lower than baseline UAS7 within 52 weeks of initiating rilzabrutinib. In some embodiments, the human patient achieves a UAS7 that is lower than baseline UAS7 within 24 weeks of initiating rilzabrutinib. In some embodiments, the human patient achieves a UAS7 that is lower than baseline UAS7 within 12 weeks of initiating rilzabrutinib. In some embodiments, the human patient achieves a UAS7 that is lower than baseline UAS7 within 4 weeks of initiating rilzabrutinib. In some embodiments, the human patient achieves a UAS7 that is lower than baseline UAS7 within 3 weeks of initiating rilzabrutinib. In some embodiments, the human patient achieves a UAS7 that is lower than baseline UAS7 within 2 weeks of initiating rilzabrutinib. In some embodiments, the human patient achieves a UAS7 that is lower than baseline UAS7 within 1 week of initiating rilzabrutinib.

In some embodiments, the human patient achieves a UAS7<6. In some embodiments, the human patient achieves a UAS7≤6 within 12 weeks of initiating rilzabrutinib treatment.

In some embodiments, the human patient achieves a UAS7 of 0. In some embodiments, the human patient achieves a UAS7 of 0 within 12 weeks of initiating rilzabrutinib treatment.

In some embodiments, the human patient achieves a UAS7 that is at least 10 points lower than baseline UAS7. In some embodiments, the human patient achieves a UAS7 that is at least 12 points lower than baseline UAS7.

In some embodiments, the human patient achieves an Itch Severity Score (ISS7) that is lower than baseline ISS7. In some embodiments, the human patient achieves an ISS7 that is lower than baseline ISS7 within 52 weeks of initiating rilzabrutinib treatment. In some embodiments, the human patient achieves an ISS7 that is lower than baseline ISS7 within 24 weeks of initiating rilzabrutinib treatment. In some embodiments, the human patient achieves an ISS7 that is lower than baseline ISS7 within 12 weeks of initiating rilzabrutinib treatment. In some embodiments, the human patient achieves an ISS7 that is lower than baseline ISS7 within 4 weeks of initiating rilzabrutinib treatment. In some embodiments, the human patient achieves an ISS7 that is lower than baseline ISS7 within 3 weeks of initiating rilzabrutinib treatment. In some embodiments, the human patient achieves an ISS7 that is lower than baseline ISS7 within 2 weeks of initiating rilzabrutinib treatment. In some embodiments, the human patient achieves an ISS7 that is lower than baseline ISS7 within 1 week of initiating rilzabrutinib treatment.

In some embodiments, the human patient achieves an ISS7 that is at least 5 points lower than a baseline ISS7. In some embodiments, the human patient achieves an ISS7 that is at least 6 points lower than a baseline ISS7.

In some embodiments, the human patient achieves a Hives Severity Score (HSS7) that is lower than baseline HSS7. In some embodiments, the human patient achieves a HSS7 that is lower than baseline HSS7 within 52 weeks of initiating rilzabrutinib treatment. In some embodiments, the human patient achieves a HSS7 that is lower than baseline HSS7 within 24 weeks of initiating rilzabrutinib treatment. In some embodiments, the human patient achieves a HSS7 that is lower than baseline HSS7 within 12 weeks of initiating rilzabrutinib treatment. In some embodiments, the human patient achieves a HSS7 that is lower than baseline HSS7 within 4 weeks of initiating rilzabrutinib treatment. In some embodiments, the human patient achieves a HSS7 that is lower than baseline HSS7 within 3 weeks of initiating rilzabrutinib treatment. In some embodiments, the human patient achieves a HSS7 that is lower than baseline HSS7 within 2 weeks of initiating rilzabrutinib treatment. In some embodiments, the human patient achieves a HSS7 that is lower than baseline HSS7 within 1 week of initiating rilzabrutinib treatment.

In some embodiments, the human patient achieves an Angioedema Activity Score (AAS7) that is lower than baseline AAS7. In some embodiments, the human patient achieves an AAS7 that is lower than baseline AAS7 within 52 weeks of initiating rilzabrutinib treatment. In some embodiments, the human patient achieves an AAS7 that is lower than baseline AAS7 within 24 weeks of initiating rilzabrutinib treatment. In some embodiments, the human patient achieves an AAS7 that is lower than baseline AAS7 within 12 weeks of initiating rilzabrutinib treatment. In some embodiments, the human patient achieves an AAS7 that is lower than baseline AAS7 within 4 weeks of initiating rilzabrutinib treatment. In some embodiments, the human patient achieves an AAS7 that is lower than baseline AAS7 within 3 weeks of initiating rilzabrutinib treatment. In some embodiments, the human patient achieves an AAS7 that is lower than baseline AAS7 within 2 weeks of initiating rilzabrutinib treatment. In some embodiments, the human patient achieves an AAS7 that is lower than baseline AAS7 within 1 week of initiating rilzabrutinib treatment.

In some embodiments, the human patient achieves a Urticaria Control Test (UCT) score that is higher than baseline UCT score. In some embodiments, the human patient achieves a UCT score that is higher than baseline UCT score within 52 weeks of initiating rilzabrutinib treatment. In some embodiments, the human patient achieves a UCT score that is higher than baseline UCT score within 24 weeks of initiating rilzabrutinib treatment. In some embodiments, the human patient achieves a UCT score that is higher than baseline UCT score within 12 weeks of initiating rilzabrutinib treatment. In some embodiments, the human patient achieves a UCT score that is higher than baseline UCT score within 8 weeks of initiating rilzabrutinib treatment.

In some embodiments, the human patient achieves a UCT score that is higher than baseline UCT score within 4 weeks of initiating rilzabrutinib treatment. In some embodiments, the human patient achieves a UCT score that is higher than baseline UCT score within 3 weeks of initiating rilzabrutinib treatment. In some embodiments, the human patient achieves a UCT score that is higher than baseline UCT score within 2 weeks of initiating rilzabrutinib treatment. In some embodiments, the human patient achieves a UCT score that is higher than baseline UCT score within 1 week of initiating rilzabrutinib treatment.

In some embodiments, the human patient achieves a UCT score≥12. In some embodiments, the human patient achieves a UCT score≥12 within 12 weeks of initiating rilzabrutinib treatment. In some embodiments, the human patient achieves a UCT score≥12 within 8 weeks of initiating rilzabrutinib treatment. In some embodiments, the human patient achieves a UCT score≥12 within 4 weeks of initiating rilzabrutinib treatment. In some embodiments, the human patient achieves a UCT score≥12 within 3 weeks of initiating rilzabrutinib treatment. In some embodiments, the human patient achieves a UCT score≥12 within 2 weeks of initiating rilzabrutinib treatment. In some embodiments, the human patient achieves a UCT score≥12 within 1 week of initiating rilzabrutinib treatment.

In some embodiments, the human patient achieves a change from baseline in health-related quality-of-life (HRQoL). In some embodiments, the human patient achieves a change from baseline in HRQoL as measured by the Patient Global Impression of Severity (PGIS). In some embodiments, the human patient achieves a change from baseline in HRQoL as measured by the PGIS within 12 weeks of initiating rilzabrutinib treatment. In some embodiments, the human patient achieves a change from baseline in HRQoL as measured by the PGIS within 8 weeks of initiating rilzabrutinib treatment. In some embodiments, the human patient achieves a change from baseline in HRQoL as measured by the PGIS within 4 weeks of initiating rilzabrutinib treatment. In some embodiments, the human patient achieves a change from baseline in HRQoL as measured by the PGIS within 3 weeks of initiating rilzabrutinib treatment. In some embodiments, the human patient achieves a change from baseline in HRQoL as measured by the PGIS within 2 weeks of initiating rilzabrutinib treatment. In some embodiments, the human patient achieves a change from baseline in HRQoL as measured by the PGIS within 1 week of initiating rilzabrutinib treatment.

In some embodiments, the human patient achieves a change from baseline in HRQoL as measured by the PGIC. In some embodiments, the human patient achieves a change from baseline in HRQoL as measured by the PGIC within 12 weeks of initiating rilzabrutinib treatment. In some embodiments, the human patient achieves a change from baseline in HRQoL as measured by the PGIC within 8 weeks of initiating rilzabrutinib treatment. In some embodiments, the human patient achieves a change from baseline in HRQoL as measured by the PGIC within 4 weeks of initiating rilzabrutinib treatment. In some embodiments, the human patient achieves a change from baseline in HRQoL as measured by the PGIC within 3 weeks of initiating rilzabrutinib treatment. In some embodiments, the human patient achieves a change from baseline in HRQoL as measured by the PGIC within 2 weeks of initiating rilzabrutinib treatment. In some embodiments, the human patient achieves a change from baseline in HRQoL as measured by the PGIC within 1 week of initiating rilzabrutinib treatment.

In some embodiments, the human patient requires rescue therapy. In some embodiments, when the human patient requires rescue therapy, the total number of days for which the human patient requires rescue therapy is lower than the total number of days for which the human patient required rescue therapy during a baseline time period of the same length. In some embodiments, when the human patient requires rescue therapy, the human patient receives rescue therapy with an H1-AH.

In some embodiments, the human patient has a history of taking one or more anti-IgE therapy prior to the start of the treatment period, optionally wherein the one or more anti-IgE therapy is omalizumab. In some embodiments, the human patient has a history of taking omalizumab prior to the start of the treatment period.

In some embodiments, the human patient has had an incomplete response to omalizumab. In some embodiments, the human patient is omalizumab naïve.

In some embodiments, the human patient has a positive basophil histamine release assay (BHRA) status. In some embodiments, the human patient has a negative BHRA status.

In some embodiments, the human patient receives concomitant treatment with an H1-AH.

In some embodiments, the human patient has previously been identified as having increased sMRGPRX2 serum levels. In some embodiments, the human patient has previously been identified as having increased mast cell MRGPRX2 expression. In some embodiments, the human patient achieves a reduction from baseline in sMRGPRX2 serum levels. In some embodiments, the human patient achieves a reduction from baseline in sMRGPRX2 serum levels within 12 weeks of initiating rilzabrutinib treatment. In some embodiments, the human patient achieves a reduction from baseline in sMRGPRX2 serum levels within 10 weeks of initiating rilzabrutinib treatment. In some embodiments, the human patient achieves a reduction from baseline in sMRGPRX2 serum levels within 8 weeks of initiating rilzabrutinib treatment. In some embodiments, the human patient achieves a reduction from baseline in sMRGPRX2 serum levels within 6 weeks of initiating rilzabrutinib treatment. In some embodiments, the human patient achieves a reduction from baseline in sMRGPRX2 serum levels within 4 weeks of initiating rilzabrutinib treatment. In some embodiments, the human patient achieves a reduction from baseline in sMRGPRX2 serum levels within 2 weeks of initiating rilzabrutinib treatment.

In some embodiments, the human patient has previously been identified as having increased IL-31 serum levels. In some embodiments, the human patient has previously been identified as having increased IL-31 serum levels as a result of increased mast cell or basophil activation. In some embodiments, the human patient achieves a reduction from baseline in IL-31 serum levels. In some embodiments, the human patient achieves a reduction from baseline in IL-31 serum levels within 12 weeks of initiating rilzabrutinib treatment. In some embodiments, the human patient achieves a reduction from baseline in IL-31 serum levels within 10 weeks of initiating rilzabrutinib treatment. In some embodiments, the human patient achieves a reduction from baseline in IL-31 serum levels within 8 weeks of initiating rilzabrutinib treatment. In some embodiments, the human patient achieves a reduction from baseline in IL-31 serum levels within 6 weeks of initiating rilzabrutinib treatment. In some embodiments, the human patient achieves a reduction from baseline in IL-31 serum levels within 4 weeks of initiating rilzabrutinib treatment. In some embodiments, the human patient achieves a reduction from baseline in IL-31 serum levels within 2 weeks of initiating rilzabrutinib treatment.

In some embodiments, the human patient has previously been identified as having increased IgG anti-TPO serum levels. In some embodiments, the human patient achieves a reduction from baseline in IgG anti-TPO serum levels. In some embodiments, the human patient achieves a reduction from baseline in IgG anti-TPO serum levels within 12 weeks of initiating rilzabrutinib treatment. In some embodiments, the human patient achieves a reduction from baseline in IgG anti-TPO serum levels within 10 weeks of initiating rilzabrutinib treatment. In some embodiments, the human patient achieves a reduction from baseline in IgG anti-TPO serum levels within 8 weeks of initiating rilzabrutinib treatment. In some embodiments, the human patient achieves a reduction from baseline in IgG anti-TPO serum levels within 6 weeks of initiating rilzabrutinib treatment. In some embodiments, the human patient achieves a reduction from baseline in IgG anti-TPO serum levels within 4 weeks of initiating rilzabrutinib treatment. In some embodiments, the human patient achieves a reduction from baseline in IgG anti-TPO serum levels within 2 weeks of initiating rilzabrutinib treatment.

In some embodiments, the human patient has previously been identified as having increased IgG anti-FCεRI serum levels. In some embodiments, the human patient has previously been identified as having increased mast cell or basophil activation as a result of increased IgG anti-FCεRI levels serum levels. In some embodiments, the human patient achieves a reduction from baseline in IgG anti-FCεRI serum levels. In some embodiments, the human patient achieves a reduction from baseline in IgG anti-FCεRI serum levels within 12 weeks of initiating rilzabrutinib treatment. In some embodiments, the human patient achieves a reduction from baseline in IgG anti-FCεRI serum levels within 10 weeks of initiating rilzabrutinib treatment. In some embodiments, the human patient achieves a reduction from baseline in IgG anti-FCεRI serum levels within 8 weeks of initiating rilzabrutinib treatment. In some embodiments, the human patient achieves a reduction from baseline in IgG anti-FCεRI serum levels within 6 weeks of initiating rilzabrutinib treatment. In some embodiments, the human patient achieves a reduction from baseline in IgG anti-FCεRI serum levels within 4 weeks of initiating rilzabrutinib treatment. In some embodiments, the human patient achieves a reduction from baseline in IgG anti-FCεRI serum levels within 2 weeks of initiating rilzabrutinib treatment.

In some embodiments, the human patient has previously been identified as having increased eosinophil levels. In some embodiments, the human patient has previously been identified as having increased mast cell activation as a result of increased eosinophil levels. In some embodiments, the human patient achieves a reduction from baseline in eosinophil levels. In some embodiments, the human patient achieves a reduction from baseline in eosinophil levels within 12 weeks of initiating rilzabrutinib treatment. In some embodiments, the human patient achieves a reduction from baseline in eosinophil levels within 10 weeks of initiating rilzabrutinib treatment. In some embodiments, the human patient achieves a reduction from baseline in eosinophil levels within 8 weeks of initiating rilzabrutinib treatment. In some embodiments, the human patient achieves a reduction from baseline in eosinophil levels within 6 weeks of initiating rilzabrutinib treatment. In some embodiments, the human patient achieves a reduction from baseline in eosinophil levels within 4 weeks of initiating rilzabrutinib treatment. In some embodiments, the human patient achieves a reduction from baseline in eosinophil levels within 2 weeks of initiating rilzabrutinib treatment.

In some embodiments, the human patient does not achieve a reduction in total serum IgG or IgE levels.

In some embodiments, the treatment period is at least 12 weeks. In some embodiments, the treatment period is at least 52 weeks.

In some embodiments, the at least one compound consists of at least one compound chosen from the (E) isomer of rilzabrutinib and pharmaceutically acceptable salts thereof. In some embodiments, the at least one compound consists of at least one compound chosen from the (Z) isomer of rilzabrutinib and pharmaceutically acceptable salts thereof. In some embodiments, the at least one compound consists of a mixture of (E) and (Z) isomers of rilzabrutinib or a pharmaceutically acceptable salt of the foregoing.

In some embodiments, the method comprises administering to the human patient about 400 mg of at least one compound chosen from rilzabrutinib and pharmaceutically acceptable salts thereof once, twice, or thrice a day. In some embodiments, the method comprises administering to the human patient about 400 mg of at least one compound chosen from rilzabrutinib and pharmaceutically acceptable salts thereof once a day. In some embodiments, the method comprises administering to the human patient about 400 mg of at least one compound chosen from rilzabrutinib and pharmaceutically acceptable salts thereof twice a day. In some embodiments, the method comprises administering to the human patient about 400 mg of at least one compound chosen from rilzabrutinib and pharmaceutically acceptable salts thereof thrice a day.

In some embodiments, the method comprises administering to the human patient rilzabrutinib once, twice, or thrice a day. In some embodiments, the method comprises administering to the human patient rilzabrutinib once a day. In some embodiments, the method comprises administering to the human patient rilzabrutinib twice a day. In some embodiments, the method comprises administering to the human patient rilzabrutinib thrice a day.

In some embodiments, the method comprises administering to the human patient 400 mg of rilzabrutinib once, twice, or thrice a day. In some embodiments, the method comprises administering to the human patient 400 mg of rilzabrutinib once a day. In some embodiments, the method comprises administering to the human patient 400 mg of rilzabrutinib twice a day. In some embodiments, the method comprises administering to the human patient 400 mg of rilzabrutinib thrice a day.

In some embodiments, the at least one compound is the (E) isomer of rilzabrutinib. In some embodiments, the at least one compound is the (Z) isomer of rilzabrutinib. In some embodiments, the at least one compound consists of a mixture of (E) and (Z) isomers of rilzabrutinib.

In some embodiments, the at least one compound is orally administered to the human patient. In some embodiments, the at least one compound is administered to the human patient in the form of at least one tablet. In some embodiments, the at least one compound is administered with water.

In some embodiments, the at least one compound is rilzabrutinib.

In some embodiments, the present disclosure provides a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof.

In some embodiments, the present disclosure provides a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof.

In some embodiments, the present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl- 4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof.

In some embodiments, the present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for the manufacture of a medicament for treating chronic spontaneous urticaria (CSU) in a human patient in need thereof.

In some embodiments, the present disclosure provides for a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof, wherein the human patient in need thereof has CSU refractory to H1 antihistamines (H1-AH) treatment.

In some embodiments, the present disclosure provides for a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof, wherein the human patient in need thereof has CSU refractory to H1 antihistamines (H1-AH) treatment.

In some embodiments, the present disclosure provides for the use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof, wherein the human patient in need thereof has CSU refractory to H1 antihistamines (H1-AH) treatment.

In some embodiments, the present disclosure provides for the use of a therapeutically effective amount of at least one compound chosen from (R)-2-[3-[4-amino-3 -(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, in the manufacture of a medicament for treating chronic spontaneous urticaria (CSU) in a human patient in need thereof, wherein the human patient in need thereof has CSU refractory to H1 antihistamines (H1-AH) treatment.

### Pharmaceutical Compositions:

In some embodiments of the present disclosure, rilzabrutinib is administered as part of a pharmaceutical composition comprising: at least one compound chosen from rilzabrutinib and pharmaceutically acceptable salts thereof; and at least one pharmaceutically acceptable excipient. In some embodiments, the pharmaceutical composition is in the form of at least one tablet.

In some embodiments, rilzabrutinib is administered in the form of a film-coated tablet.

In some embodiments of the present disclosure, rilzabrutinib is administered in the form of at least one tablet comprising: at least one compound chosen from rilzabrutinib and pharmaceutically acceptable salts thereof; and at least one pharmaceutically acceptable excipient. In some embodiments, rilzabrutinib is administered in the form of at least one tablet comprising: at least one compound chosen from rilzabrutinib and pharmaceutically acceptable salts thereof; at least one filler; at least one disintegrant; at least one lubricant; and at least one film coating.

In some embodiments, rilzabrutinib is administered with a glass of water.

The proportion and nature of any pharmaceutically acceptable excipient may be determined by the chosen route of administration and standard pharmaceutical practice. Except insofar as any conventional pharmaceutically acceptable excipient is incompatible with rilzabrutinib, such as by producing any undesirable biological effect or otherwise interacting in a deleterious manner with any other component(s) of the pharmaceutically composition, its use is contemplated to be within the scope of this disclosure.

Some non-limiting examples of materials which may serve as pharmaceutically acceptable excipients include: (1) sugars, such as, e.g., lactose, glucose, and sucrose; (2) starches, such as, e.g., corn starch and potato starch; (3) cellulose and its derivatives, such as, e.g., sodium carboxymethyl cellulose, ethyl cellulose, and cellulose acetate; (4) powdered tragacanth; (5) malt; (6) gelatin; (7) talc; (8) excipients, such as, e.g., cocoa butter and suppository waxes; (9) oils, such as, e.g., peanut oil, cottonseed oil, safflower oil, sesame oil, olive oil, corn oil, and soybean oil; (10) glycols, such as, e.g., propylene glycol; (11) polyols, such as, e.g., glycerin, sorbitol, mannitol, and polyethylene glycol; (12) esters, such as, e.g., ethyl oleate and ethyl laurate; (13) agar; (14) buffering agents, such as, e.g., magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Remington: The Science and Practice of Pharmacy, 21st edition, 2005, ed. D.B. Troy, Lippincott Williams & Wilkins, Philadelphia, and Encyclopedia of Pharmaceutical Technology, eds. J. Swarbrick and J. C. Boylan, 1988-1999, Marcel Dekker, New York also discloses additional non-limiting examples of pharmaceutically acceptable excipients, as well as known techniques for preparing and using the same.

One skilled in the art can readily select the proper form and route of administration depending upon the disorder or condition to be treated, the stage of the disorder or condition, and other relevant circumstances.

### EXAMPLES

The following example is intended to be illustrative and is not meant in any way to limit the scope of the disclosure.

### Abbreviations

AAS7 Angioedema activity score over 7 days
AE Adverse event
AESI Adverse event of special interest
API Active pharmaceutical ingredient
ALP Alkaline phosphatase
ALT Alanine aminotransferase
aPTT Activated partial thromboplastin time
AST Aspartate aminotransferase
BHRA Basophil histamine release assay
bid/BID Twice daily (morning and evening)
BM Biomarker
BMI Body mass index
BTK Bruton's Tyrosine Kinase
CD Cluster of differentiation
CIU Chronic idiopathic urticaria
CMH Cochran-Mantel Haenzel test
CPK Creatine phosphokinase
D Day
DB Double-blind
DLQI Dermatology Life Quality Index
ECG Electrocardiogram
e-diary Electronic diary
ED Early discontinuation
EOT End of treatment
EOS End of study
FU Follow-up
HbA1c Glycated hemoglobin
H1-AH H1 antihistamine
HIV Human immunodeficiency virus
HRQoL Health-related quality of life
HSS Hives severity score
IgE Immunoglobulin E
IgG Immunoglobulin G
IgM Immunoglobulin M
IL Interleukin
IMP Investigational medicinal product
INR International normalized ratio
ISS Itch severity score
ITP Immune thrombocytopenia
LDH Lactate dehydrogenase
LS Least squares
MCID Minimal clinically important difference
MI Multiple imputation
MRGPRX2 Mas-related G-protein coupled receptor member X2
N Number
OCS Oral corticosteroids
OLE Open-label extension phase
OR Odds ratio
PBMC Peripheral blood mononuclear cells
PGIC Patient global impression of change
PGIS Patient global impression of severity
PK Pharmacokinetic
PRO Patient-reported outcome
qd/QD Once a day
QOL Quality of Life
QPM Once each evening
R Randomization
RRD Response rate difference
SAE Serious adverse event
TEAE Treatment-emergent adverse event
TB Tuberculosis
TID Three times daily/thrice daily
TPO Thyroperoxidase
UAS7 Urticaria activity score over 7 days
UCT Urticaria control test
ULN Upper limit of normal
V Visit
W Week
WOCBP Woman of childbearing potential
WOCF Worst observation carried forward

### Example 1: a Randomized, Double-Blind, Placebo-Controlled, Multi-Center, Dose-Ranging Phase 2 Study of Rilzabrutinib Followed by an Open-Label Extension Phase in Patients with Moderate-to-Severe Chronic Spontaneous Urticaria (CSU) Who Remain Symptomatic Despite the Use of H1 Antihistamine Treatments

### Study Design

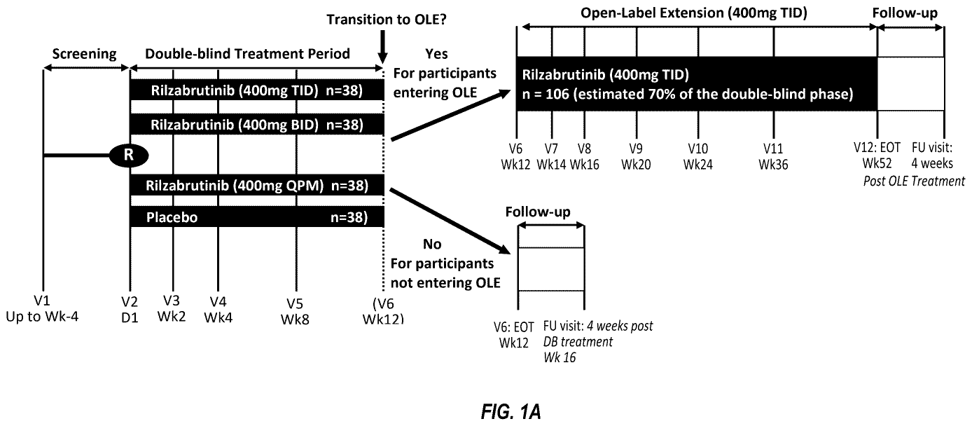

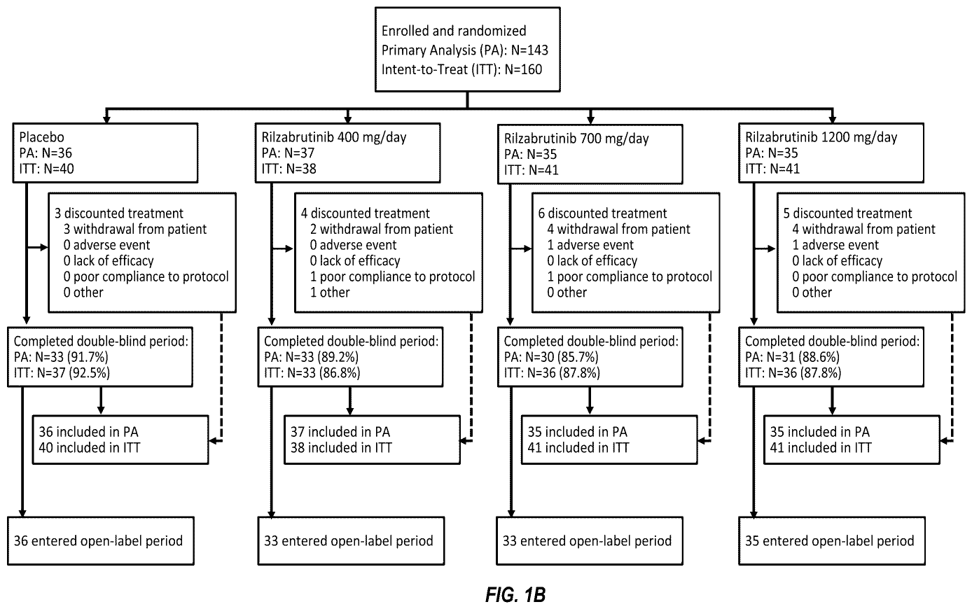

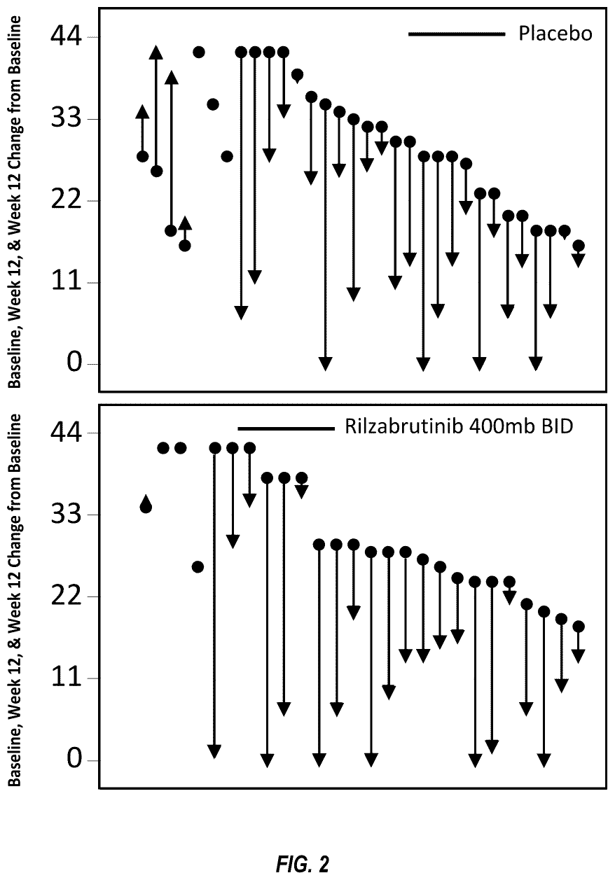

This study is a 52-week sequential Phase 2 study on the effects of rilzabrutinib in adult participants with moderate-to-severe CSU who remain symptomatic despite the use of H1 antihistamine (H1-AH) treatment. The study comprises a 12 week, randomized, double-blind, placebo-controlled, multi-center, dose-ranging efficacy and safety phase, followed by a 40 week open label extension (OLE) phase (see FIG. 1).

Patients exhibited at least moderate disease activity (see inclusion criteria) despite the use of H1-AH. It was expected that ~40% of patients would have concomitant angioedema. Participants were to be randomized 1:1:1:1 to one of three doses of rilzabrutinib or placebo. The randomization was to be stratified by region and prior use of omalizumab.

The study is to evaluate the effect of rilzabrutinib on itch and hives, scored individually or as a composite score; on angioedema activity; on participant-reported urticaria control; and on HRQoL.

The study consists of three phases and an optional OLE phase. The phases are as follows:
Screening period (up to 4 weeks)
Double-blind IMP treatment period (12 weeks): participants were randomized 1:1:1:1 to one of the following treatments: rilzabrutinib 400 mg TID; rilzabrutinib 400 mg BID with matched placebo; rilzabrutinib 400 mg QPM with matched placebo; or matched placebo
Optional OLE phase (40 weeks): for participants who completed the 12-week double-blind phase, eligible participants were given the option to continue to an OLE phase of the study. Participants in the OLE will be given rilzabrutinib 400 mg TID (the dose may be modified based on the 12-week safety and efficacy data) (all non-Germany countries). In Germany, participants in the OLE will be administered rilzabrutinib 400 mg BID.
Follow-up period (4 weeks)

During the double-blind IMP treatment phase of this study, participants were to continue their established standard of care background therapy with an approved long-acting H1-AH, at up to 4-fold the recommended dose. If a participant was on an H1-AH dose higher than 4-fold the recommended dose at the screening visit, the Investigator may have adjusted the participant's dose to within the stipulated dose range at the screening visit. For participants on stable doses of nonstudy-approved H1-AH, Investigators may have switched participants to an equivalent dose of a study-approved H1-AH maintenance medication at the screening visit. Participants should have continued their screening dose of H1-AH throughout the double-blind phase of the study, unless they experienced a disease flare. Participants should also continue their baseline dose of H1-AH for the first 12 weeks of the OLE phase of the study. After 12 weeks in the OLE phase, Investigators may, at their discretion, reduce participants' H1-AH dose, but the antihistamine daily dose still may not exceed 4-fold the recommended dose (all non-Japan countries) or 2-fold the recommended dose (Japan only).

In the case that participants are considered for rescue therapy, participants on 1-, 2- or 3-fold the recommended H1-AH may receive additional doses of their study-approved H1-AH as rescue medication, as long as their daily dose does not exceed 4-fold the recommended daily dose of the H1-AH (all non-Japan countries) or 2-fold the recommended daily dose of the H1-AH (Japan only). At the investigator's discretion, participants may be prescribed a short course of oral corticosteroids (OCS) as rescue therapy during the treatment, OLE and follow up periods. Rescue therapy should start with an increase in H1-AH whenever possible, followed by a short course of OCS if needed. For analysis of urticaria activity (including itch and hives individually), angioedema activity, participant-reported urticaria control and HRQoL, data collected after OCS use will be set to missing and the worse post-baseline values before OCS therapy will be used.

The end of the study in the 12 week double-blind phase of the study is defined as the date of the last follow-up visit of the last participant in the double-blind phase of study. Participants were considered to have completed the 12 week double-blind phase of the study if they had completed the 12 week phase of the study, including the last follow-up visit.

The end of the study in the 40 week open-label phase of the study is defined as the date of the last follow-up visit of the last participant in the open-label phase of the study. Participants will be considered to have completed the 40 week open-label phase of the study once they have completed the 40-week phase of the study including the last follow-up visit.

The assessments used in this study are standard endpoints in evaluation of disease activity and response to therapy in CSU. Clinically, CSU is characterized by the spontaneous and recurrent appearance of pruritic hives. The proposed primary endpoints were the UAS7 at Week 12 in EU and EU reference countries and the ISS7 in the US. Regardless of where a participant was from, all assessments were the same. The UAS7 score is a composite score containing both the HSS7 and the ISS7. This allowed a global assessment of the two key components of urticaria: hives and itch. The UAS7 score has been well-established as a prospective measure of CSU activity and has been used in a number of clinical studies as a primary outcome measure (Agache et al. 2021; Hawro et al. 2018; Maurer et al. 2019). As an independent score, ISS7 has also been well established and widely accepted as a primary outcome measure for CSU activity (Maurer et al. 2013).

Angioedema is present in up to 40.3% of patients with CSU and can have a significant negative impact on quality of life (Sussman et al. 2018). This study aimed to assess the effect of rilzabrutinib on angioedema activity. The weekly angioedema activity score (AAS7) was proposed as a tertiary endpoint. The AAS7 score is a validated tool determine angioedema activity and has previously been used in CSU clinical studies (Maurer et al. 2019; Weller et al. 2013).

This study also aimed to assess disease control from the perspective of participants. To this end, the UCT was proposed as a tertiary endpoint in this study. The UCT is a validated tool which has become the standard Patient-Reported Outcome (PRO) tool to determine disease control in CSU and other forms of chronic urticaria (Weller et al. 2014; Zuberbier et al. 2018). In addition to disease activity scores, this study aimed to assess the impact of rilzabrutinib on HRQoL in CSU. The dermatology life quality index (DLQI) was proposed as a tertiary endpoint in this study. The DLQI is a standard well-studied HRQoL score which has been validated for use in chronic urticaria (Lennox et al. 2004).

Blood samples were also collected through Week 12 to evaluate the change in baseline in various biomarkers, including sMRGPRX2, IgG anti-TPO, IgG anti-FCεRI, IgG anti-IgE, and IL-31.

In sum, the proposed primary, secondary and tertiary endpoints were intended to allow a determination of the efficacy of rilzabrutinib on disease activity, disease symptoms, and HRQoL in participants with CSU.

TABLE 1
Objectives and Endpoints.

| | |
|---|---|
| Objectives | Endpoints |

Primary

| | |
|---|---|
| To demonstrate the efficacy of rilzabrutinib | Change from baseline in weekly UAS7 at |
| in study participants with CSU who remain | Week 12 (except US and US reference |
| symptomatic despite the use of H1-AH | countries) |
| | For US and US reference countries only: |
| | change from baseline in weekly ISS7 at |
| | Week 12 |

Secondary

| | |
|---|---|
| To demonstrate the efficacy of rilzabrutinib | Change from baseline in UAS7 at Week 4 |
| on urticaria activity composite endpoint and | Change from baseline in ISS7 at Week 12 |
| itch or hives, separately, at various time | (except US and US reference countries) |
| points | For US and US reference countries only: |
| | change from baseline in UAS7 at Week 12 |
| | Change from baseline in weekly HSS7 at |
| | Week 12 |
| | Proportion of participants with UAS7 ≤ 6 |
| | at Week 12 |
| | Proportion of participants with UAS7 = 0 |
| | at Week 12 |
| To evaluate safety outcome measures | Incidence of TEAEs, SAEs, AESIs, and |
| | withdrawals due to TEAEs during the DB |
| | period and OLE |
| To assess the plasma PK of rilzabrutinib in | Plasma PK concentrations of rilzabrutinib in |
| participants with CSU | participants with CSU |

Tertiary

| | |
|---|---|
| To demonstrate the efficacy of rilzabrutinib | Change from baseline in UAS7 at all time |
| on urticaria activity composite endpoint and | points (onset of action is assessed by the |
| itch or hives, separately, at various time | first p < 0.05 that remains significant at |
| points | subsequent measures until Week 12) |
| | Time to UAS7 minimal clinically |
| | important difference (MCID) in response |
| | (improvement from baseline = 10) |
| | Proportion of participants with UAS7 ≤ 6 |
| | at all time points |
| | Change from baseline in ISS7 at all time |
| | points (onset of action is assessed by the |
| | first p < 0.05 that remains significant at |
| | subsequent measures until Week 12) |
| | Time to ISS7 minimal clinically important |
| | difference (MCID) in response |
| | (improvement from baseline ≥ 5) |
| | Change from baseline in HSS7 at all time |
| | points (onset of action is assessed by the |
| | first p < 0.05 that remains significant at |
| | subsequent measures until Week 12) |
| | Change from baseline in UAS7 at Week |
| | 24 and Week 52 |
| | Change from baseline in ISS7 at Week 24 |
| | and Week 52 |
| | Change from baseline in HSS7 at Week |
| | 24 and Week 52 |
| To demonstrate the efficacy of rilzabrutinib | Change from baseline in AAS7 at all time |
| on angioedema | points (onset of action is assessed by the |
| | first p < 0.05 that remains significant at |
| | subsequent measures until Week 12) |
| | Change from baseline in AAS7 at Week |
| | 24 and Week 52 |
| To demonstrate the efficacy of rilzabrutinib | Change from baseline in UCT at Week 4, |
| on urticaria control | at Week 8 and at Week 12 |
| | Proportion of well-controlled participants |
| | (UCT ≥ 12) at Week 4, at Week 8 and at |
| | Week 12 |
| | Change from baseline in UCT at Week 24 |
| | and Week 52 |
| To demonstrate improvement in health- | Change from baseline in HRQoL as |
| related quality-of-life and overall disease | measured by Dermatology Life Quality |
| status and severity | Index (DLQI) at Week 4, at Week 8 and |
| | Week 12; by Patient Global Impression of |
| | Severity (PGIS) at Week 2, Week 4, Week |
| | 8, and Week 12; by Patient Global |
| | Impression of Change (PGIC) at Week 2, |
| | Week 4, Week 8, and Week 12 |
| | Change from baseline in DLQI at Week |
| | 24 and week 52 |
| To evaluate the ability of rilzabrutinib in | Time-to-event of participants receiving |
| reducing the need for oral corticosteroids | rescue therapy with OCS for CSU during |
| | the double-blind phase |
| | Proportion of participants receiving rescue |
| | therapy with OCS for CSU during the |
| | double-blind phase |
| | Total number of days each participant |
| | requires rescue therapy with OCS for CSU |
| | during the double-blind phase |

### Study Population

Inclusion criteria are summarized in Table 2. Participants were eligible to be included in the study only if they met all of the inclusion criteria.

TABLE 2
Inclusion Criteria.

| | |
|---|---|
| Age | Participants must have been 18-80 years of age (inclusive) at the |
| | time of signing the informed consent |
| Disease | Participants must have had a diagnosis of CSU refractory to H1- |
| characteristics | AH at the time of randomization, as defined by all of the |
| | following: |
| | Diagnosis of CSU ≥ 3 months prior to screening visit (Visit 1) |
| | The presence of itch and hives for ≥ 6 consecutive weeks at any |
| | time prior to screening visit (Visit 1) despite the use of H1-AH |
| | during this time period. |
| | Participants must have been using a study defined H1-AH for |
| | CSU treatment. For participants on stable doses of non-study- |
| | approved H1-AH, investigators may switch participants to an |
| | equivalent dose of a study-approved H1-AH maintenance |
| | medication. Note: participants should have remained on their |
| | prescreening non-sedating H1-AH dose. Only up to 4-fold the |
| | recommended dose was allowed. If participants were on dose |
| | higher than 4-fold the recommended dose at screening, the |
| | Investigator could have adjusted the participant dose to the |
| | stipulated range at the screening visit (Visit 1). Participants were |
| | to remain on this dose of antihistamine for at least 3 consecutive |
| | days before screening assessment of UAS7 and ISS7 is initiated. |
| | Participants who were omalizumab-naïve OR with prior |
| | omalizumab use, including omalizumab-incomplete responders |
| | (as determined by the Site Investigator and defined as inadequate |
| | control of CSU symptoms despite treatment with omalizumab 300 |
| | mg every 4 weeks for at least 3 months (minimum 3 injections). |
| | Note: Documentation of incomplete response must have been |
| | documented in the patient's medical records). |
| | During the 7 days before randomization, patients must have had |
| | a UAS7 ≥ 16, ISS7 ≥ 8, with no missing e-diary UAS7 or ISS7 |
| | records |
| Sex, | Male participants must have agreed to the following during the |
| contraceptive/barrier | study intervention period and for at least 13 weeks after the last |
| method and | administration of study intervention: |
| pregnancy testing | refrain from donating or cryopreserving sperm |
| requirements | abstain from heterosexual or homosexual intercourse as their |
| | preferred and usual lifestyle (i.e., must have been abstinent on a |
| | long term and persistent basis) and agree to remain abstinent; or |
| | use contraception and a barrier (i.e., a male condom and an |
| | additional highly effective contraceptive method when having |
| | sexual intercourse with a WOCBP who is not currently pregnant; |
| | or a male condom when engaging in any activity that allows for |
| | passage of ejaculate to another person) |
| | Female participants must not have been pregnant or |
| | breastfeeding and must have met the following criteria: |
| | be a WONCBP; or agree to use a highly-effective contraceptive |
| | method (i.e., a contraceptive method with a failure rate of < 1% |
| | per year), preferably with low user dependency, during the study |
| | intervention period. The contraceptive method must be effective |
| | before starting the intervention and for at least 4 weeks after the |
| | last administration of study intervention. The participant must |
| | agree not to donate or cryopreserve eggs (ova/oocytes) for the |
| | purpose of reproduction during this period). Eligible WOCBP |
| | participants must have a negative highly sensitive pregnancy test |
| | (serum as required by local regulations) within 28 days before the |
| | first administration of study intervention. If a urine test cannot be |
| | confirmed as negative (e.g., an ambiguous result), a serum |
| | pregnancy test is required. In such cases, the participant must be |
| | excluded from participation of the serum pregnancy result is |
| | positive. |
| Informed consent | Participants must have been capable of giving signed informed |
| | consent. |
| Additional criteria | Participants must have been willing and able to complete a daily |
| | symptom e-diary for the duration of the study |

Exclusion criteria are summarized in Table 3. Participants were excluded from the study if any of the criteria applied.

TABLE 3
Exclusion Criteria.

| | |
|---|---|
| Medical Conditions | Participants must not have had any of the following medical |
| | conditions: |
| | Clearly defined underlying etiology for CUs other than CSU |
| | (main manifestation being physical urticaria). |
| | Presence of skin morbidities other than CSU that may interfere |
| | with the assessment of the study outcomes |
| | Active atopic dermatitis (AD) |
| | Severe concomitant illness(es) that, in the Investigator's |
| | judgment, would adversely affect the patient's participation in the |
| | study. |
| | Known or suspected immunodeficiency, or otherwise recurrent |
| | infections of abnormal frequency or prolonged duration |
| | suggesting an immune compromised status, as judged by the |
| | Investigator. |
| | History of serious infections requiring intravenous (IV) therapy |
| | with the potential for recurrence, or currently active moderate to |
| | severe infection at Screening (Grade 2 or higher), including active |
| | coronavirus disease 2019 (COVID-19) |
| | Live vaccine except Bacille Calmette Guerin-vaccination within |
| | 28 days prior to Day 1 or plan to receive one during the trial; |
| | Bacille Calmette Guerin-vaccination within 12 months prior to |
| | Screening. |
| | Active malignancy or history of malignancy within 5 years |
| | before Day 1, |
| | Conditions that may predispose the participant to excessive |
| | bleeding |
| | An uncontrolled disease state as judged by the Investigator, such |
| | as asthma, psoriasis, or an inflammatory bowel disease, etc. that is |
| | typically treated with oral and/or parenteral corticosteroids |
| Prior/concurrent | Previous use of a BTK inhibitor |
| clinical study | Use of any investigational drug (or current use of an |
| experience | investigational device) within the 30 days before Day 1, or at least |
| | 5 times the respective elimination half-life time (whichever is |
| | longer) |
| | Previous exposure to another investigative drug for CSU |
| Diagnostic | Positive HIV antibody test |
| Assessments | Presence of hepatitis B surface antigen (HBsAg) or hepatitis B |
| | core antibody (HBcAb) with positive DNA test result at screening |
| | or within 3 months prior to the screening visit (all non-Japan |
| | countries) |
| | Presence of hepatitis B surface antigen (HBsAg) with/without |
| | positive HBV DNA test result or presence of hepatitis B core |
| | antibody (HBcAb) or presence of hepatitis B surface antibody |
| | (HBsAb) with positive HBV DNA test result at screening or |
| | within 3 months prior to the screening visit (Japan only) |
| | Positive hepatitis C antibody test result at screening or within 3 |
| | months prior to the screening visit. |
| | Exclusions related to tuberculosis infection (TBI) |
| | Any laboratory abnormalities at the screening visit |

### Screen Failures

Screen failures are defined as participants who consent to participate in the clinical study but are not subsequently randomized. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure reasons, eligibility criteria, and any SAE.

If a participant failed to complete 2 or fewer days of their UAS7 evaluation at screening, or if their e-diary device failed, that participant may have been rescreened once. However, if a participant did not meet the minimum UAS7 or ISS7 score in screening, that participant was not eligible for rescreening.

In cases where original screen failure was due to reasons expected to change at re-screening and based upon the Investigator's clinical judgment, the participant may have been rescreened one time for this study after notification of the Sponsor. Rescreened participants were to be assigned a new participant number for the re-screening event. There was no requirement for a waiting period between the screen-failure and the re-screening. A new consent form must have been signed and Visit 1 procedures must have been repeated for re-screened participants.

### Study Intervention(s) Administered

Study intervention is defined as any investigational intervention(s), marketed product(s), placebo, or medical device(s) intended to be administered to a study participant according to the study protocol. Tables 4 and 5 provide an overview of interventions administered in the present study.

Consecutive doses were to be administered approximately 6 hours apart (and not less than 4 hours apart).

TABLE 4
Overview of Study Interventions Administered

| | | |
|---|---|---|
| Intervention label | Rilzabrutinib | Placebo |
| Intervention name | Rilzabrutinib | Placebo |
| Type | Drug | Drug |
| Dose formulation | Tablets tablets/caplets) | Placebo to match rilzabrutinib |
| | | tablets (/caplets) |
| Unit dose strength(s) | 400 mg | 0 mg |
| Dosage level(s) | 400 mg (1 tablet) BID, QPM, | 1 tablet BID, QPM, or TID |
| | or TID | |
| Route of administration | Oral | Oral |
| Use | Experimental | Experimental |
| IMP or NIMP | IMP | IMP |
| Packaging and labeling | During the double-blind | During the double-blind |
| | phase, IMP was to be | phase, IMP was to be |
| | provided in wallets of 1 | provided in wallets of 1 |
| | blister pack, and each wallet | blister pack, and each wallet |
| | was to be labeled as required | was to be labeled as required |
| | per country requirement; | per country requirements |
| | during the open-label phase, | |
| | IMP was to be supplied in | |
| | white plastic bottles, and each | |
| | bottle was to be labeled as | |
| | required per country | |
| | requirement | |
| Current/Former name(s) or | Rilzabrutinib | Placebo |
| aliases | | |

TABLE 5
Arms and Associated Interventions

| Arm name | Placebo | 400 mg QPM | 400 mg BID | 400 mg TID |
|---|---|---|---|---|
| Associated | Rilzabrutinib | Rilzabrutinib | Rilzabrutinib | Rilzabrutinib |
| interventions | or placebo | or placebo | or placebo | or placebo |
| (intervention | | | | |
| label(s)) | | | | |

### Noninvestigational Medicinal Products

Participants were to continue their established standard of care background therapy with a long-acting, non-sedating H1-AH during the study. For participants on stable doses of non-study-approved H1-AH, investigators may have switched participants to an equivalent dose of a study-approved H1-AH maintenance medication. Only up to 4-fold (all non-Japan countries) or 2-fold (Japan only) the recommended dose was allowed. If participants were on dose higher than 4-fold the recommended dose at screening, the Investigator could have adjusted the participant dose to the stipulated range at the screening visit (Visit 1). Participants must have remained on this dose of antihistamine for at least 3 consecutive days before screening assessment of UAS7 and ISS7 is initiated. Participants were to continue to take the same daily dose throughout the study unless they experience a flare for which rescue therapy may have been initiated. During the OLE phase, participants are to also continue their baseline dose of H1-AH for the first 12 weeks of the OLE phase. After 12 weeks in the OLE phase, Investigators may, at their discretion, decide to reduce or discontinue participants' maintenance antihistamines dose, but the antihistamine daily dose still may not exceed 4-fold the recommended dose; the dose of rilzabrutinib will remain the same.

Because rilzabrutinib is a mild inhibitor of CYP3A, only the following study-approved antihistamines and recommended were allowed as background therapy to minimize the possibility of rilzabrutinib altering their pharmacokinetics: cetirizine (10 mg QD); levocetirizine dihydrochloride (5 mg QD); fexofenadine (60 mg BID or 180 mg QD); desloratadine (5 mg QD); and bilastine (20 mg QD).

### Dose Modification

Rilzabrutinib dose modification was not allowed during the double-blind phase. For the OLE phase, the dose may be modified based on the 12 week safety and efficacy data.

### Concomitant Therapy

Any medication or vaccine (including over-the-counter or prescription medicines, recreational drugs, vitamins, and/or herbal supplements) that the participant was receiving at the time of enrollment or received during the study must have been recorded along with the reason for use, the dates of administration (including the start and end dates), and dosage information (including dose and frequency). The sponsor should have been contacted if there were any questions regarding concomitant or prior therapy.

Participants were to abstain from taking nonprescription drugs (including vitamins and dietary or herbal supplements) within 2 weeks or 5 half-lives (whichever was longer) before the start of study intervention until completion of the follow-up visit, unless, in the opinion of the Investigator, the medication would not interfere with the study. New or chronic prescription medications could have been used if needed at the discretion of the Investigator. The Medical Monitor was to be contacted if there were any questions regarding new or chronic medications.

Clinically relevant drugs that are substrates of CYP3A, including those considered to be sensitive CYP3A substrates were permitted. Appropriate caution was to be used when co-administering sensitive CYP3A substrates with rilzabrutinib and a benefit-risk assessment was to be conducted for each medication. Consideration was also to have been given to avoidance of high doses, dose reduction, or replacement of sensitive and narrow therapeutic index CYP3A substrate drug. Systemic strong and moderate inhibitors (including foods such as grapefruit juice) and inducers of CYP3A were to be avoided.

Medications that have not been listed as exclusion criteria were permitted for use during the trial. These include the following:
Oral corticosteroid rinses (mouth washes) are allowed.
H2 antihistamine (H2-AH) receptor blocking drugs were permitted provided they were taken once daily only and were taken 2-3 hours after administration of the evening rilzabrutinib or placebo dose.
Antacids were permitted provided they were given 2 hours or more apart from rilzabrutinib or placebo dose.
COVID-19 vaccines

Participants were to continue their established standard of care background therapy with a long acting, non-sedating H1-AH (defined as noninvestigational medicinal product (NIMP)). During the OLE phase, participants should also continue their baseline dose of H1-AH for the first 12 weeks of the OLE phase. After 12 weeks in the OLE phase, Investigators may, at their discretion, decide to reduce or discontinue participants' maintenance antihistamines dose; the dose of rilzabrutinib will remain the same. In addition, participants should continue to take only allowed concomitant therapy, as defined for the first phase of the study (i.e., 12-week double-blind period).

A list of prohibited concomitant therapies is provided in Table 6.

TABLE 6
Prohibited Concomitant Therapy

| Drug Category | Drug Name |
|---|---|
| Immunosuppressants | Mycophenolate mofetil |
| | Azathioprine |
| | Methotrexate |
| | Cyclosporine |
| | Dapsone |
| | Anakinra (Kineret) |
| | Etanercept (Enbrel) |
| | Topical corticosteroids, topical calcineurin inhibitors, |
| | and topical antihistamines (non-Italy countries only) |
| | Systemic corticosteroids (except for OCS used as rescue |
| | Immunosuppressant drugs |
| Biologics or targeted-synthetic | Omalizumab (non-Italy countries only) |
| disease modifiers | Infliximab |
| | Adalimumab |
| | Golimumab |
| | Abatacept |
| | Tocilizumab |
| | Sarilumab |
| | Certolizumab |
| | Secukinumab |
| | IFN-γ |
| | JAK inhibitors |
| | Dupilumab |
| | Other biologic or targeted-synthetic disease modifiers |
| Anti-CD20 drugs | Rituximab |
| | Ofatumumab |
| | Other long-acting biologics |
| Antifibrinolytic | Tranexamic acid |
| | Epsilon-aminocaproic acid |
| Leukotriene receptor antagonists | Montelukast |
| and 5-lipoxygenase inhibitors | Zafirlukast |
| (Note: OK to continue if used for | Zileuton (non-Italy countries only) |
| an indication other than CSU) | |
| H2 receptor antagonists (Note: OK | Cimetidine |
| to continue if used for an | Ranitidine |
| indication other than CSU) | Famotidine |
| | Nizatidine |
| | Roxatidine |
| | Lafutidine |
| | Other H2 Receptor antagonists |
| Proton pump inhibitors | Omeprazole |
| | Esomeprazole |
| | Lansoprazole |
| | Pantoprazole |
| | Other proton pump inhibitors |
| Immunomodulator therapy | Intravenous immunoglobulin (IVIG); subcutaneous |
| | immunoglobulin (SCIG) (non-Italy countries only) |
| | Plasmapheresis |
| Antidepressant | Doxepin |
| Systemic strong-to-moderate | Strong CYP3A inducers: apalutamide, carbamazepine, |
| inhibitors and inducers of | enzalutamide, mitotane, phenytoin, rifampin, St. John's |
| cytochrome P450 CYP3A | wort |
| | Moderate CYP3A inducers: Bosentan, efavirenz, |
| | etravirine, phenobarbital, primidone |
| | Strong CYP3A inhibitors: boceprevir, clarithromycin, |
| | cobicistat, danoprevir/ritonavir, elvitegravir/ritonavir, |
| | grapefruit juice, idelalisib, indinavir/ritonavir, |
| | itraconazole, ketoconazole, lopinavir/ritonavir, |
| | nefazodone, nelfinavir, paritaprevir/ritonavir and |
| | ombitasvir and/or dasabuvir, posaconazole, ritonavir, |
| | saquinavir/ritonavir, telaprevir, telithromycin, |
| | tipranavir/ritonavir, troleandomycin, voriconazole |
| | Moderate CYP3A inhibitors: aprepitant, ciprofloxacin, |
| | conivaptan, crizotinib, cyclosporine, dronedarone, |
| | erythromycin, fluconazole, fluvoxamine, imatinib, |
| | tofisopam, verapamil |

This table provides a list of the most common medications within the classes of prohibited concomitant drugs but is not exhaustive.

### Rescue Medicine

As rescue medication, up to 4-fold of the approved dose of the participants usual H1-AH could have been given on days where pruritus and wheal development were severe. The total daily H1-AH dose was not to exceed 4-fold the recommended dose during the screening, treatment and follow-up period.

The initial maintenance antihistamine dose should have remained stable throughout the 12-week double-blind phase and the first 12 weeks of the OLE phase, and participants were to continue their maintenance dose once rescue treatment is no longer required. After 12 weeks in the OLE phase, Investigators may, at their discretion, decide to reduce or discontinue participants' maintenance antihistamines dose, but the antihistamine daily dose still may not exceed 4-fold the recommended dose.

If needed, or if unable to increase H1-AH as an initial step for rescue therapy, participants may have been prescribed a short course of OCS. After any use of OCS, participants would have needed to seek immediate medical attention to ensure that the initial reaction had been successfully controlled and/or to evaluate for additional therapeutic steps. In order to ensure consistency, when possible, it was recommended to use OCS for 5 to 7 days with a starting dose of oral prednisone 40 mg (or clinically comparable OCS) followed by taper per the Investigator's judgment. The use of OCS was to be delayed, if possible, for at least 8 weeks following the initiation of the investigational treatment. The date and time of OCS administration as well as the name and dosage regimen of the OCS was to be recorded.

Unless permanently discontinued from the study, all participants were to complete the scheduled study visits and assessments whether or not they completed study treatment and whether or not they received rescue treatment for CSU. Investigators were to make every attempt to conduct efficacy and safety assessments immediately before administering any rescue treatment. An unscheduled visit may have been used for this purpose, if necessary. Participants who received rescue therapy with OCS during the study treatment were considered “treatment failures” for the analysis of all efficacy endpoints.

### Efficacy Assessments

Efficacy data was to be collected via electronic devices. The e-diary was to be used for daily recording of PRO such as the UAS7 and AAS7 questionnaires, and use of H1-AH medication. This device was to be dispensed at Screening (Visit 1), including instructions for use. Additionally, participants were to be instructed on the use of the device.

Participants were to fill in UCT, DLQI, PGIC, and PGIS questionnaires during their site visit.

### Urticaria Activity Score

The UAS is a validated PRO measure. The daily UAS is the sum of the daily Hive Severity Score (HSS, ranging from 0=None to 3=more than 50 hives) and the daily Itch Severity Score (ISS, ranging from 0=None to 3=intense), the 2 key urticaria signs and symptoms which are wheals and itch. The daily UAS scores range from 0 to 6 point/day. Daily UAS scores are summed over 7-day period to create the UAS7, ranging from 0 to 42, and are composed of the HSS7 and ISS7 components. The UAS7 is an established and widely accepted PRO tool to prospectively measure CSU activity (Mlynek et al. 2008). It has been used in most clinical trials in CSU in recent years as a main outcome parameter and medical practice (Maurer et al. 2013; Metz et al. 2020). A minimal important difference (MID) value ranging from 9.5 to 10.5 has been defined to help interpretation of the change in score in CSU participants (Hawro et al. 2018; Hollis et al. 2018; Mathias et al. 2012). Patients were to complete the UAS using the e-diary. The e-diary was to be used once daily on the same time of each day recording of participant's answers to the urticaria activity score over 7 days (UAS7) questionnaire. For participants who entered the OLE phase, their answers to the UAS7 questionnaire were also to be collected from Week 20 to Week24 and from Week 48 to Week 52.

### Angioedema Activity Score

The AAS is a validated PRO measure that assesses angioedema activity (Weller et al. 2013). The AAS is a diary in which participants document on a daily basis the presence or absence of angioedema during the past 24 hours. If angioedema is present, participants answer 5 additional questions about the time of the day the swelling episode occurred, and the severity and impact on daily functioning and appearance this swelling episode has had. Each AAS item is scored between 0 and 3 points, that is, the minimum and maximum daily AASs are 0 and 15 points. The daily AASs are summed up to 7-day scores (AAS7), with 7-day scores ranging from 0 to 105 (Weller et al. 2013). A MID of the AAS7 of around 8 points has been established (Weller et al. 2013). Patients were to complete the AAS using the e-diary. The e-diary was to be used once daily on the same time of each day recording of participant's answers to the angioedema activity score over 7 days (AAS7) questionnaire. For participants who entered the OLE phase, their answers to the AAS7 questionnaire were also to be collected from Week 20 to Week24 and from Week 48 to Week 52.

### Urticaria Control Test

The UCT is a validated PRO measure for assessing urticaria control (Weller et al. 2014) based on 4 items (severity of pruritus and wheals urticaria symptoms; frequency of treatment being not sufficient; QoL impairment; overall urticarial control). Each item is rated on a 5-point Likert-type scale (scored with 0 to 4 points). Low scores indicate high disease activity and low disease control. The UCT total score is calculated by adding all 4 individual item scores. Accordingly, the minimum and maximum UCT scores are 0 and 16, with a score of 16 points indicating complete disease control (Weller et al. 2014). The recall period is the previous 4 weeks. Patients were to complete the UCT questionnaire during their site visit.

### Dermatology Life Quality Index

The DLQI is a PRO developed to measure dermatology-specific HRQoL in adult participants (Finlay and Khan 1994). The instrument comprises 10 items assessing the impact of skin disease on participants' HRQoL over the previous week with an average completion time of 126 seconds (Loo et al. 2003). The items cover symptoms, leisure activities, work/school or holiday time, personal relationships including intimate, the side effects of treatment, and emotional reactions to having a skin disease. It is a validated questionnaire used in clinical practice and clinical trials (Chernyshov 2019). Response scale is a 4-point Likert scale (0=“not at all” and 3=“very much”) for 9 items. The remaining 1 item about work/studying asks whether work/study has been prevented and then (if “No”) to what degree the skin condition has been a problem at work/study; the item is rated on a 3-point Likert scale (“Not at all” to “A lot”). Overall scoring ranges from 0 to 30, with a high score indicative of a poor HRQoL. Using an integrated analysis of distribution and anchor-based approaches using the change in DLQI total score and participant-assessed itch severity scores, the MID for the DLQI in participants with CIU was reported to be in the range of 2.24 to 3.10 points (Shikiar et al. 2005). The recall period is the previous week. Patients were to complete the DLQI questionnaire during their site visit.

### Patient Global Impression of Change of CSU Disease and Patient Global Impression of Severity of CSU Disease

The PGIS is a 1-item measure that asks participants to provide a self-assessment of their overall CSU severity for the past week on a 4 point scale. Response choices are: “None”, “Mild”, “Moderate” and “Severe”. The PGIC is a 1-item measure that asks the participant to provide a self-assessment of overall change in their CSU since the participant started taking the study medication on a 7-point scale. The response options range from “Very Much Better” to “Very Much Worse.” Patients were to complete questionnaires for the 2 items during their site visit.

### Adverse Events (AEs), Serious Adverse Events (SAEs), and Other Safety Reporting

An AE is defined as any untoward medical occurrence in a patient or clinical study participant, temporally associated with the use of study intervention, whether or not considered related to the study intervention¹. 1 An AE can therefore be any unfavorable and unintended sign (including an abnormal laboratory finding), symptom, or disease (new or exacerbated) temporally associated with the use of study intervention.

Events meeting the AE definition include any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the Investigator (i.e., not related to progression of underlying disease), e.g., events that are symptomatic; events that require either corrective treatment or consultation; events that lead to IMP discontinuation or modification of dosing; events that fulfill a seriousness criterion; and/or events defined as an AESI. Events meeting the AE definition further include the exacerbation of a chronic or intermittent pre-existing condition, including either an increase in frequency and/or intensity of the condition; a new condition detected or diagnosed after study intervention administration even though it may have been present before the start of the study; signs, symptoms, or clinical sequelae of a suspected drug-drug interaction; and/or signs, symptoms, or clinical sequelae of a suspected overdose of either study intervention or a concomitant medication.

An SAE is defined as any AE that, at any dose, results in death, is life-threatening, requires inpatient hospitalization or prolongation of existing hospitalization, results in persistent or significant disability/incapacity, or is a congenital anomaly/birth defect.

The term “life-threatening” in the definition of “serious” refers to an event in which the participant was at risk of death at the time of the event. It does not refer to an event, which hypothetically might have caused death if it were more severe.

In general, hospitalization signifies that the participant has been admitted (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for elective treatment of a pre-existing condition that did not worsen from baseline is not considered an AE.

The term “disability” means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.

Other situations may also have been considered SAEs. Examples of such events include invasive or malignant cancers, intensive treatment in an emergency room or at home for allergic bronchospasm, blood dyscrasias or convulsions that did not result in hospitalization, or development of drug dependency or drug abuse.

The Investigator was to make an assessment of intensity for each AE and SAE reported during the study and assign it to one of the following categories:
Mild: an event that was easily tolerated by the participant, causing minimal discomfort and not interfering with everyday activities.
Moderate: an event that caused sufficient discomfort to interfere with normal everyday activities.
Severe: an event that prevented normal everyday activities. An AE that was assessed as severe should not have been confused with an SAE. “Severe” is a category used for rating the intensity of an event; and both AEs and SAEs can be assessed as severe. An event is defined as “serious” when it meets at least one of the predefined outcomes as described in the definition of an SAE, and not when it is rated as severe.

### Adverse Events of Special Interest

An AESI is an AE (serious or nonserious) of scientific and medical concern specific to the Sponsor's product or program, for which ongoing monitoring and immediate notification by the Investigator to the Sponsor is required. Such events may have required further investigation in order to characterize and understand them. AESIs may have been added, modified, or removed during a study by protocol amendment. Exemplary AESIs are listed below.
Pregnancy of a female participant entered in the study, or pregnancy occurring in a female partner of a male participant entered in the clinical trial. Such a pregnancy was to be qualified as an SAE only if it fulfilled one of the seriousness criteria. In the event of pregnancy in a female participant, the IMP was to be discontinued. Follow-up of the pregnancy in a female participant or in a female partner of a male participant was mandatory until the outcome had been determined.
Symptomatic overdose (serious or nonserious) with an IMP or NIMP. An overdose (accidental or intentional) with the IMP was an event suspected by the Investigator or spontaneously notified by the participant (not based on systematic pills count) and defined as a single ingestion of a dose equal to or greater than 1,200 mg (≥3 times the indicated single dose of 400 mg), provided that it had been taken at once or over 2-hour period. An overdose (accidental or intentional) with the NIMP was an event suspected by the Investigator or spontaneously notified by the participant (not based on systematic pills count) and defined as at least twice the maximum allowed daily dose, within the intended therapeutic interval. For H1-AH only, an overdose is defined as any dose that exceeds the maximum allowed daily dose in this protocol.
Increase in ALT. For participants with an increase in ALT>3×ULN, report the ALT>3×ULN together with total bilirubin value including normal ranges and confirm the elevation within 72 hours.
Other project specific AESIs. This may have included severe infections, including opportunistic infections; tuberculosis or initiation of medications for suspected tuberculosis; diagnosed and biologically proven SARS-CoV-2 infection; major hemorrhagic events, including symptomatic bleeding in a critical area or organ such as the central nervous system (CNS), or intraocular bleeding, resulting in an SAE; severe or serious AEs (or non-serious AEs with a severity level consistent with Grade 3 Common Terminology Criteria for AE (CTCAE) of cytopenia v 5.0; and atrial fibrillation.

### Pharmacokinetics

Plasma samples were to be obtained for PK characterization of rilzabrutinib. Selected residual plasma samples may have been utilized to assess PK of relevant metabolites of rilzabrutinib. Concentrations at each time point were to be reported.

### Biomarkers

Collection of biological samples for other biomarker research was also part of this study. The following samples for biomarker research were required and were to be collected from all participants in this study:
Serum and plasma samples for soluble biomarkers and cellular tests. Analytes included IgG anti-IgE, IgG anti-FcεRI, IgG anti-TPO, IgE anti-TPO, IL-31, and sMRGPRX2.
Blood samples for immune cell phenotyping, including measurement of eosinophil levels
Blood samples for BTK occupancy in peripheral blood mononuclear cells (PBMCs) at selected sites

For each patient, serum samples were to be collected on each of four visits (Visits 2, 4, 6, and end of study/follow up).

Serum analyte testing was to be performed using the antibodies and antigens listed below:
hIgG anti-FcεRI (Cat. No. #130-122-308; Miltenyi)
hIgG anti-IgE-biotin (Cat. No. #130-117-938; Miltenyi)
Anti-IgE-HRP (Cat. No. #5220-0329; SeraCare)
Anti-hIgG HRP (Cat. No. #109-035-008; Jackson IR)
Purified TPO (Cat. No. #043/20; In.vent Diagnostica)
Recombinant human FcεRIα (HIS-tag) (Cat. No. #FCA-H5228; AcroBiosystems)
Human IgE (myeloma) (Cat. No. #401152; Merck Millipore)
IvIg (Kiovig) (Cat. No. #LE-07-43020; Baxter AG/Shire)
IgE anti-TPO (SP1.4-IgE anti-TPO purified from hybridoma supernatants) (Guo et al. 1996)

Serum analyte testing was to be performed using the following commercial assays:
sMRGPRX2 ELISA (Cat. No. #MBS933336; MyBioSource)
IL-31 HTRF assay (Cat. No. #62HIL31PEG; Cisbio/PerkinElmer)
IgG anti-TPO ELISA (Cat. No. #ORG503; Orgentec Diagnostica GmbH)

All chemical and biological reagents used for serum analyte testing were of analytical grade and were used without further purification. Any water used for dilution, e.g., for the washing buffer, was to be deionized and obtained directly from the facilities installation.

The following commercial reagents and consumables were to be used:
ECL prime (Cat. No. #10308449; Cytiva/FisherScientific)
Streptavidin HRP (Cat. No. #DY998; R&D Systems)
PBS (Cat. No. #14190094; Gibco)
Protein-free blocking buffer (Cat. No. #927-90001; LI-COR)
TritonX-100 (Cat. No. #T8532; SigmaAldrich)
Tween-20 (Cat. No. #M147; VWR LifeSciences)
NaCl
MaxiSorp 384 well plates (Cat. No. #P6491; Nunc/SigmaAldrich)

Assay buffer was to be prepared using PBS, Tween-20, TritonX-100, and NaCl. TBS was to comprise 10 mM Tris, 150 mM Nacl, pH 7.4. TBST was to comprise TBS and Tween-20.

Laboratory equipment was to include a Multimode Plate Reader (VICTOR5; Perkin Elmer).

If dilutions were to be prepared, the same dilutions were to be prepared twice from each sample. These were to be measured on different plates. Each plate was to include a standard curve. Samples were to be measured twice for all biomarkers except for sMRGPRX2. Mean measurement values were to be calculated, and the concentration of the biomarker was to be calculated using GraphPad Prism 9 software (4-PL model).

Biomarker assays were performed as described below.

For the IgG anti-IgE ELISA, MaxiSorp plates were coated overnight with 1 g/mL human IgE antigen and blocked with 10% protein-free blocking buffer diluted in assay buffer. Following the addition of serum samples (1:100 dilution in assay buffer) or the hIgG anti-IgE standard (1:2 serial dilution in assay buffer), the plates were incubated at room temperature. Next, the anti-IgG-HRP detection antibody was added to all samples. Development was performed using ECL-prime solution, and chemiluminescent signal intensity was measured on the Victor5 multimode plate reader.

For the IgG anti-FcεRI ELISA, MaxiSorp plates were coated overnight with 1 g/mL recombinant human FcεRIα and blocked with 10% protein-free blocking buffer diluted in assay buffer. Following the addition of serum samples (1:100 in assay buffer) or the hIgG anti-FcεRI standard (1:2 serial dilution in assay buffer), the plates were incubated at room temperature. Next, the anti-IgG-HRP detection antibody was added to all samples. Development was performed using ECL-prime solution and chemiluminescent signal intensity was measured on the Victor5 multimode plate reader.

The IgG anti-TPO ELISA was performed according to the manufacturer's instructions.

For the IgE anti-TPO ELISA, plates were coated with purified human TPO and blocked with 1% fetal bovine serum (FBS) diluted in TBST. Serum samples (1:5 in TBS) or the SP1.4-IgE anti-TPO standard (1:2 serial dilution in assay buffer) were incubated at room temperature. Next, the anti-IgE-HRP antibody was added to all samples. Development was performed using ECL-prime solution and chemiluminescent signal intensity was measured on the Victor5 multimode plate reader.

The IL-31 HTRF assay was performed according to the manufacturer's instructions.

For the sMRGPRX2 ELISA, measurements were performed according to the manufacturer's instructions. Single measurements were obtained for all samples.

Assay data ranges are described below.
sMRGPRX2: 0.1 ng/mL-10 ng/mL
IgG anti-FCεRI: 0-10000 arbitrary units (AU)
IgG anti-TPO: 0-3000 international units (IU)
IgE anti-TPO: 0-500 AU
IgG anti-IgE: 0-100000 AU
IL-31: 0.1 ng/mL-20 ng/mL

### Statistical Considerations

In this study, the statistical hypotheses for comparing each of rilzabrutinib groups against placebo on the primary endpoint of change from baseline in ISS7 at Week 12 (for US and US reference countries), and the primary endpoint of change from baseline in UAS7 at Week 12 (for countries except US and US reference countries) were as follows:
Null hypothesis H0: there is no treatment difference between rilzabrutinib and placebo.
Alternative hypothesis H1: there is a treatment difference between rilzabrutinib and placebo.

The statistical hypotheses to be tested on secondary efficacy endpoints could be specified similarly as those on the primary endpoint.

Participants were randomized to the intervention group in a ratio of 1:1:1:1.

An absolute change of 5 in ISS7 score is considered the minimal clinically important difference (MCID) and an absolute change of 10 in UAS7 score is considered the MCID. Based upon an SD of 7, a change of 5 in the ISS7 would correspond to an effect size of approximately 0.71. Based upon a SD of 14, a change of 10 in the UAS7 would correspond to an effect size of approximately 0.71. Based on this assumption, plus the assumption of a 15% dropout rate before Week 12, it was estimated that 38 patients per group would provide approximate 80% power to detect an effect size of 0.71 or higher between the rilzabrutinib arm and placebo using a 2-sided t-test with alpha=0.05. This sample size estimate applies to both ISS7 (primary endpoint for US and US reference countries) and UAS7 (primary endpoint for all countries except US and US reference countries).

The sample size calculations were performed using EAST 6.5.

The randomization was to be stratified by region and prior use of omalizumab (yes or no). Note: prior use of omalizumab=yes represents patients with prior omalizumab use, including omalizumab-incomplete responders, and prior use of omalizumab=no represents omalizumab-naïve patients. Patients with prior omalizumab use, including omalizumab-incomplete responders were only to be randomized to the 400 mg BID, 400 mg TID and placebo arms, and they were to be capped at up to 15% of the patients in that arm.

### Populations for Analyses

The populations for analyses are defined in Table 7.

TABLE 7
Populations for Analyses

| Population | Description |
|---|---|
| Screened | All participants who signed the ICF |
| Randomized | All participants from screened population |
| | who were allocated to a randomized |
| | intervention by IRT regardless of whether the |
| | intervention was received |
| Intent-to-treat (ITT) | All randomized participants. Participants were |
| | to be analyzed according to the intervention |
| | allocated by randomization. |
| Omalizumab-naïve | All randomized omalizumab-naïve |
| | participants. Participants were to be analyzed |
| | according to the intervention they actually |
| | received. |
| Safety | All randomized participants who took at least |
| | one dose of study intervention. Participants |
| | were to be analyzed according to the |
| | intervention they actually received |
| Pharmacokinetic | All randomized and treated participants |
| (PK) | (safety population) without important |
| | deviation related to IMP administration, and |
| | with at least one post-baseline PK sample |
| | with adequate documentation of dosing and |
| | sampling dates and times. Participants having |
| | received only placebo were not to be part of |
| | the PK population. Participants were to be |
| | analyzed according to the intervention they |
| | actually received. |
| Open-label | All participants who received at least one |
| extension (OLE) | dose of intervention during the open-label |
| | extension phase. |

The efficacy endpoints analyses were to be primarily based on the omalizumab-naïve population. The ITT population was to be used as the supplementary analysis for selected efficacy endpoints.

### Primary Endpoints

The primary endpoint was change from baseline in UAS7 at Week 12 (except US and US reference countries). For US and US reference countries, the primary endpoint was change from baseline in ISS7 at Week 12.

The summary of primary estimand for the primary endpoint is provided in Table 8.

TABLE 8
Primary Estimand for the Primary Endpoint

| | |
|---|---|
| Treatment | Rilzabrutinib 400 mg QPM, 400 mg BID, 400 mg TID, and |
| | placebo, along with established standard of care background |
| | therapy with a long-acting, non-sedating H1-AH. |
| Population | Omalizumab-naïve |
| Variables | Change from baseline in UAS7 at Week 12 (except US and US |
| | reference countries). Change from baseline in ISS7 at Week 12 |
| | (for US and US reference countries only). |
| Intercurrent events and | For participants who discontinued the study intervention, all |
| handling strategy for | data collected after discontinuation were to be used in the |
| missing data handlings | analysis (treatment policy strategy). After discontinuation of |
| | the study intervention due to lack of efficacy prior to Week 12, |
| | WOCF approach was to be used to impute missing data if |
| | needed. After discontinuation of the study intervention due to |
| | reasons other than lack of efficacy prior to Week 12, MI |
| | approach was to be used to impute missing endpoint values, |
| | and this MI was to use all participants excluding participants |
| | who had taken selected prohibited medications and/or rescue |
| | medications prior to Week 12. |
| | For participants who took selected prohibited medications |
| | and/or rescue medications prior to Week 12, data were to be |
| | set to missing values after the medication usage, and the |
| | participant's worst postbaseline value on or before the time of |
| | the medication usage was to be used to impute missing |
| | endpoint values. For participants whose postbaseline values |
| | were all missing, the participant's baseline value was to be |
| | used to impute the missing endpoint value (hypothetical |
| | strategy). |
| Population-level | An analysis of covariance model with intervention groups, |
| summary | region (combined countries) and baseline value of the endpoint |
| | as covariates was to be used. Statistical inference obtained |
| | from all imputed data was to be combined using Rubin's rule. |
| | Descriptive statistics including number of subjects, mean, |
| | standard error, and LS mean changes (and standard error) |
| | score will be provided. In addition, difference of the |
| | rilzabrutinib group against placebo in LS means and the |
| | corresponding 95% CI was to be provided along with the p- |
| | values. |

### Supplementary Analysis

The primary endpoint was also to be analyzed on ITT population. In addition, descriptive statistics for patients with prior omalizumab use, including omalizumab-incomplete responders, were to be provided.

As-observed analysis (including all data after taking the prohibited and/or rescue medications) and other supplementary analyses were to be performed to provide additional insights into the understanding of the treatment effect.

In addition to the comparison between each dose level of rilzabrutinib and placebo in a pairwise manner, a pooled analysis of the BID and TID doses may have been performed as appropriate.

### Secondary Endpoint(s)

The following secondary endpoints were to be analyzed using the same approach as for the primary endpoint:
Change from baseline in UAS7 at Week 4
Change from baseline in UAS7 at Week 12 (for US and US reference countries)
Change from baseline in ISS7 at Week 12 (except US and US reference countries)
Change from baseline in HSS7 at Week 12

The following responder secondary endpoints were to be analyzed using the Cochran-Mantel-Haenszel test adjusted by region:
Proportion of participants with UAS7<6 at Week 12
Proportion of participants with UAS7=0 at Week 12

Participants who received selected prohibited medications and/or rescue medications were to be considered as non-responders for time points after the medication usage. For other participants, all available data including those collected during the off-treatment period were to be used to determine the responder/non-responder status. Missing data were to be considered as non-responders.

No multiplicity adjustment for the secondary efficacy endpoints was to be made. A pooled analysis of the BID and TID doses may have been performed as appropriate.

### Safety Analysis

All safety analysis was to be based on the safety population according to the intervention group to which the participants are exposed. The safety analysis was to be conducted separately for double-blind period and open-label period.

### Example 2: Double-Blind Treatment Phase Results

A study was initiated to evaluate the effect of rilzabrutinib treatment in patients with moderate-to-severe chronic spontaneous urticaria (CSU) whose disease was not controlled with H1-antihistamines (H1-AH) alone. Over the course of this 12-week, double-blind, placebo-controlled study, N=160 patients were administered one of three doses of rilzabrutinib (400 mg QD, 400 mg BID, or 400 mg TID) or a placebo.

Patients exhibited significant improvement across all disease components, including urticaria activity, itch severity, and hives. These improvements, which were observed from Week 1, were sustained through Week 12. Moreover, improvements were observed in both the intent-to-treat (ITT) and omalizumab-naïve participant populations.

Primary endpoints for this study included the change from baseline in weekly urticaria activity score (UAS7) at Week 12 (all non-US countries) and the change from baseline in weekly itch severity score (ISS7) at Week 12 (US only). Secondary and tertiary endpoints are described in Table 1.

For patients receiving 400 mg TID rilzabrutinib, the study met the primary endpoints for both non-US countries and the US. Additionally, for patients receiving 400 mg TID rilzabrutinib, the study met several secondary endpoints, including the proportion of participants with well-controlled disease (UAS7<6) at Week 12 and the percent change from baseline in weekly hives severity score (HSS7) at Week 12.

Evidence of a dose-response relationship was observed for patients treated with rilzabrutinib. While there was no difference between patients receiving 400 mg QPM rilzabrutinib and patients receiving the placebo, patients receiving 400 mg BID rilzabrutinib showed positive improvements across all endpoints when compared to patients receiving the placebo.

Moreover, rilzabrutinib was generally safe, with no evidence of new safety concerns or dose-related AEs.

### Participant Demographics

The 160 patients enrolled in the phase 2 study were characterized by the demographic information provided in Tables 9-12. Of the 160 patients randomized, 142 (88.75%) completed the double-blind period. Baseline demographics and disease characteristics were generally well balanced across treatment arms (Tables 9 and 10). The omalizumab-naïve patient population was similar to the ITT population.

The median age of enrolled patients was 44. At enrollment, patients had CSU for a median duration of 3.0 years, and 11.6% had previously received omalizumab (patients with prior omalizumab use, including omalizumab-incomplete responders). Additionally, 36.9% of patients had a history of angioedema.

TABLE 9
Demographics and Participant Characteristics at Baseline (Randomized Population)

| | | | | |
|---|---|---|---|---|
| | Rilzabrutinib | Rilzabrutinib | Rilzabrutinib | |
| Placebo | 400 mg QPM | 400 mg BID | 400 mg TID | All |
| (N = 40) | (N = 38) | (N = 41) | (N = 41) | (N = 160) |

Age (years)

| | | | | | |
|---|---|---|---|---|---|
| Number | 40 | 38 | 41 | 41 | 160 |
| Mean (SD) | 40.2 (14.0) | 42.1 (12.7) | 45.5 (13.4) | 48.5 (12.2) | 44.1 (13.4) |
| Median | 39.5 | 41.0 | 43.0 | 49.0 | 44.0 |
| Q1; Q3 | 27.0; 50.5 | 33.0; 50.0 | 34.0; 57.0 | 40.0; 56.0 | 33.0; 54.5 |
| Min; Max | 19; 70 | 20; 66 | 24; 73 | 22; 77 | 19; 77 |

Age group (years) [n (%)]

| | | | | | |
|---|---|---|---|---|---|
| Number | 40 | 38 | 41 | 41 | 160 |
| 18-39 | 20 (50.0) | 16 (42.1) | 17 (41.5) | 10 (24.4) | 63 (39.4) |
| 40-64 | 18 (45.0) | 21 (55.3) | 22 (53.7) | 27 (65.9) | 88 (55.0) |
| ≥65 | 2 (5.0) | 1 (2.6) | 2 (4.9) | 4 (9.8) | 9 (5.6) |

Age group (years) [n (%)]

| | | | | | |
|---|---|---|---|---|---|
| Number | 40 | 38 | 41 | 41 | 160 |
| <44 (Median) | 25 (62.5) | 20 (52.6) | 21 (51.2) | 13 (31.7) | 79 (49.4) |
| ≥44 (Median) | 15 (37.5) | 18 (47.4) | 20 (48.8) | 28 (68.3) | 81 (50.6) |

Region^{a} [n (%)]

| | | | | | |
|---|---|---|---|---|---|
| Number | 40 | 38 | 41 | 41 | 160 |
| Asia | 9 (22.5) | 10 (26.3) | 8 (19.5) | 8 (19.5) | 35 (21.9) |
| Latin America | 14 (35.0) | 14 (36.8) | 11 (26.8) | 9 (22.0) | 48 (30.0) |
| Eastern Europe | 3 (7.5) | 4 (10.5) | 3 (7.3) | 5 (12.2) | 15 (9.4) |
| Western Countries | 14 (35.0) | 10 (26.3) | 19 (46.3) | 19 (46.3) | 62 (38.8) |

Sex [n (%)]

| | | | | | |
|---|---|---|---|---|---|
| Number | 40 | 38 | 41 | 41 | 160 |
| Male | 14 (35.0) | 10 (26.3) | 16 (39.0) | 8 (19.5) | 48 (30.0) |
| Female | 26 (65.0) | 28 (73.7) | 25 (61.0) | 33 (80.5) | 112 (70.0) |

Race [n (%)]

| | | | | | |
|---|---|---|---|---|---|
| Number | 40 | 38 | 41 | 41 | 160 |
| White | 31 (77.5) | 27 (71.1) | 33 (80.5) | 30 (73.2) | 121 (75.6) |
| Black or African American | 0 | 0 | 0 | 0 | 0 |
| Asian | 9 (22.5) | 11 (28.9) | 8 (19.5) | 10 (24.4) | 38 (23.8) |
| Japanese | 2 (5.0) | 3 (7.9) | 2 (4.9) | 3 (7.3) | 10 (6.3) |
| Native Hawaiian or Other Pacific | 0 | 0 | 0 | 0 | 0 |
| Islander | | | | | |
| American Indian or Alaska Native | 0 | 0 | 0 | 1 (2.4) | 1 (0.6) |
| Multiple | 0 | 0 | 0 | 0 | 0 |
| Not reported | 0 | 0 | 0 | 0 | 0 |
| Unknown | 0 | 0 | 0 | 0 | 0 |

Ethnicity [n (%)]

| | | | | | |
|---|---|---|---|---|---|
| Number | 40 | 38 | 41 | 41 | 160 |
| Hispanic or Latino | 13 (32.5) | 17 (44.7) | 12 (29.3) | 11 (26.8) | 53 (33.1) |
| Not Hispanic or Latino | 27 (67.5) | 21 (55.3) | 28 (68.3) | 28 (68.3) | 104 (65.0) |
| Not reported | 0 | 0 | 1 (2.4) | 1 (2.4) | 2 (1.3) |
| Unknown | 0 | 0 | 0 | 1 (2.4) | 1 (0.6) |

Weight (kg)

| | | | | | |
|---|---|---|---|---|---|
| Number | 40 | 38 | 41 | 41 | 160 |
| Mean (SD) | 74.86 (18.00) | 72.31 (16.45) | 75.68 (18.99) | 74.52 (16.37) | 74.38 (17.38) |
| Median | 72.65 | 67.35 | 75.00 | 75.00 | 72.90 |
| Q1; Q3 | 58.60; 87.40 | 60.50; 81.90 | 61.00; 87.00 | 64.00; 81.50 | 60.75; 85.00 |
| Min; Max | 44.0; 110.6 | 48.9; 118.0 | 49.8; 141.5 | 43.4; 114.8 | 43.4; 141.5 |

Weight group (kg) [n (%)]

| | | | | | |
|---|---|---|---|---|---|
| Number | 40 | 38 | 41 | 41 | 160 |
| <72.9 (Median) | 21 (52.5) | 23 (60.5) | 20 (48.8) | 16 (39.0) | 80 (50.0) |
| ≥72.9 (Median) | 19 (47.5) | 15 (39.5) | 21 (51.2) | 25 (61.0) | 80 (50.0) |

Weight group (kg) [n (%)]

| | | | | | |
|---|---|---|---|---|---|
| Number | 40 | 38 | 41 | 41 | 160 |
| <60 | 11 (27.5) | 7 (18.4) | 9 (22.0) | 8 (19.5) | 35 (21.9) |
| ≥60-<90 | 20 (50.0) | 28 (73.7) | 24 (58.5) | 28 (68.3) | 100 (62.5) |
| ≥90 | 9 (22.5) | 3 (7.9) | 8 (19.5) | 5 (12.2) | 25 (15.6) |

BMI (kg/m²)

| | | | | | |
|---|---|---|---|---|---|
| Number | 40 | 38 | 41 | 41 | 160 |
| Mean (SD) | 27.04 (5.73) | 26.01 (5.48) | 27.01 (6.18) | 27.69 (5.25) | 26.95 (5.65) |
| Median | 25.30 | 25.20 | 24.90 | 27.80 | 25.90 |
| Q1; Q3 | 23.65; 31.00 | 21.70; 30.50 | 22.60; 30.10 | 24.50; 29.20 | 22.90; 29.95 |
| Min; Max | 17.9; 42.4 | 18.4; 38.6 | 17.3; 43.2 | 17.6; 42.2 | 17.3; 43.2 |

BMI group (kg/m²) [n (%)]

| | | | | | |
|---|---|---|---|---|---|
| Number | 40 | 38 | 41 | 41 | 160 |
| <25 | 17 (42.5) | 18 (47.4) | 21 (51.2) | 12 (29.3) | 68 (42.5) |
| ≥25-<30 | 12 (30.0) | 10 (26.3) | 9 (22.0) | 21 (51.2) | 52 (32.5) |
| ≥30 | 11 (27.5) | 10 (26.3) | 11 (26.8) | 8 (19.5) | 40 (25.0) |

^{a}Asia: South Korea, Taiwan, Japan; Latin America: Argentina, Chile; Eastern Europe: Poland; Western Countries: Spain, Italy, Germany, Canada, Greece, Netherlands.

TABLE 10
Demographics and Participant Characteristics at Baseline (Omalizumab-Naïve Population)

| | | | | |
|---|---|---|---|---|
| | Rilzabrutinib | Rilzabrutinib | Rilzabrutinib | |
| Placebo | 400 mg QPM | 400 mg BID | 400 mg TID | All |
| (N = 36) | (N = 37) | (N = 35) | (N = 35) | (N = 143) |

Age (years)

| | | | | | |
|---|---|---|---|---|---|
| Number | 36 | 37 | 35 | 35 | 143 |
| Mean (SD) | 39.3 (13.7) | 41.9 (12.9) | 45.5 (13.6) | 48.7 (12.8) | 43.8 (13.6) |
| Median | 38.0 | 41.0 | 44.0 | 49.0 | 43.0 |
| Q1; Q3 | 27.0; 49.5 | 33.0; 50.0 | 33.0; 57.0 | 40.0; 59.0 | 31.0; 54.0 |
| Min; Max | 19; 70 | 20; 66 | 24; 73 | 22; 77 | 19; 77 |

Age group (years) [n (%)]

| | | | | | |
|---|---|---|---|---|---|
| Number | 36 | 37 | 35 | 35 | 143 |
| 18-39 | 19 (52.8) | 16 (43.2) | 15 (42.9) | 8 (22.9) | 58 (40.6) |
| 40-64 | 15 (41.7) | 20 (54.1) | 18 (51.4) | 23 (65.7) | 76 (53.1) |
| ≥65 | 2 (5.6) | 1 (2.7) | 2 (5.7) | 4 (11.4) | 9 (6.3) |

Age group (years) [n (%)]

| | | | | | |
|---|---|---|---|---|---|
| Number | 36 | 37 | 35 | 35 | 143 |
| <44 (Median) | 24 (66.7) | 20 (54.1) | 17 (48.6) | 11 (31.4) | 72 (50.3) |
| ≥44 (Median) | 12 (33.3) | 17 (45.9) | 18 (51.4) | 24 (68.6) | 71 (49.7) |

Region^{a} [n (%)]

| | | | | | |
|---|---|---|---|---|---|
| Number | 36 | 37 | 35 | 35 | 143 |
| Asia | 9 (25.0) | 10 (27.0) | 8 (22.9) | 8 (22.9) | 35 (24.5) |
| Latin America | 13 (36.1) | 14 (37.8) | 10 (28.6) | 8 (22.9) | 45 (31.5) |
| Eastern Europe | 3 (8.3) | 4 (10.8) | 3 (8.6) | 4 (11.4) | 14 (9.8) |
| Western Countries | 11 (30.6) | 9 (24.3) | 14 (40.0) | 15 (42.9) | 49 (34.3) |

Sex [n (%)]

| | | | | | |
|---|---|---|---|---|---|
| Number | 36 | 37 | 35 | 35 | 143 |
| Male | 13 (36.1) | 9 (24.3) | 13 (37.1) | 7 (20.0) | 42 (29.4) |
| Female | 23 (63.9) | 28 (75.7) | 22 (62.9) | 28 (80.0) | 101 (70.6) |

Race [n (%)]

| | | | | | |
|---|---|---|---|---|---|
| Number | 36 | 37 | 35 | 35 | 143 |
| White | 27 (75.0) | 26 (70.3) | 27 (77.1) | 25 (71.4) | 105 (73.4) |
| Black or African American | 0 | 0 | 0 | 0 | 0 |
| Asian | 9 (25.0) | 11 (29.7) | 8 (22.9) | 10 (28.6) | 38 (26.6) |
| Japanese | 2 (5.6) | 3 (8.1) | 2 (5.7) | 3 (8.6) | 10 (7.0) |
| Native Hawaiian or Other Pacific | 0 | 0 | 0 | 0 | 0 |
| Islander | | | | | |
| American Indian or Alaska Native | 0 | 0 | 0 | 0 | 0 |
| Multiple | 0 | 0 | 0 | 0 | 0 |
| Not reported | 0 | 0 | 0 | 0 | 0 |
| Unknown | 0 | 0 | 0 | 0 | 0 |

Ethnicity [n (%)]

| | | | | | |
|---|---|---|---|---|---|
| Number | 36 | 37 | 35 | 35 | 143 |
| Hispanic or Latino | 12 (33.3) | 16 (43.2) | 11 (31.4) | 10 (28.6) | 49 (34.3) |
| Not Hispanic or Latino | 24 (66.7) | 21 (56.8) | 23 (65.7) | 23 (65.7) | 91 (63.6) |
| Not reported | 0 | 0 | 1 (2.9) | 1 (2.9) | 2 (1.4) |
| Unknown | 0 | 0 | 0 | 1 (2.9) | 1 (0.7) |

Weight (kg)

| | | | | | |
|---|---|---|---|---|---|
| Number | 36 | 37 | 35 | 35 | 143 |
| Mean (SD) | 73.33 (17.55) | 72.64 (16.55) | 74.13 (19.95) | 72.62 (15.08) | 73.17 (17.19) |
| Median | 71.75 | 67.70 | 71.90 | 75.00 | 71.90 |
| Q1; Q3 | 58.20; 84.60 | 60.70; 81.90 | 56.70; 84.00 | 62.00; 81.50 | 60.20; 82.70 |
| Min; Max | 44.0; 110.6 | 48.9; 118.0 | 49.8; 141.5 | 43.4; 110.0 | 43.4; 141.5 |

Weight group (kg) [n (%)]

| | | | | | |
|---|---|---|---|---|---|
| Number | 36 | 37 | 35 | 35 | 143 |
| <72.9 (Median) | 20 (55.6) | 22 (59.5) | 19 (54.3) | 15 (42.9) | 76 (53.1) |
| ≥72.9 (Median) | 16 (44.4) | 15 (40.5) | 16 (45.7) | 20 (57.1) | 67 (46.9) |

Weight group (kg) [n (%)]

| | | | | | |
|---|---|---|---|---|---|
| Number | 36 | 37 | 35 | 35 | 143 |
| <60 | 11 (30.6) | 7 (18.9) | 9 (25.7) | 8 (22.9) | 35 (24.5) |
| ≥60-<90 | 18 (50.0) | 27 (73.0) | 19 (54.3) | 24 (68.6) | 88 (61.5) |
| ≥90 | 7 (19.4) | 3 (8.1) | 7 (20.0) | 3 (8.6) | 20 (14.0) |

BMI (kg/m²)

| | | | | | |
|---|---|---|---|---|---|
| Number | 36 | 37 | 35 | 35 | 143 |
| Mean (SD) | 26.48 (5.32) | 26.22 (5.41) | 26.49 (6.07) | 27.19 (4.82) | 26.59 (5.38) |
| Median | 25.25 | 25.40 | 24.50 | 27.10 | 25.40 |
| Q1; Q3 | 23.35; 30.05 | 21.80; 30.50 | 22.40; 31.30 | 24.20; 29.10 | 22.60; 29.60 |
| Min; Max | 17.9; 39.7 | 18.4; 38.6 | 17.3; 43.2 | 17.6; 41.4 | 17.3; 43.2 |

BMI group (kg/m²) [n (%)]

| | | | | | |
|---|---|---|---|---|---|
| Number | 36 | 37 | 35 | 35 | 143 |
| <25 | 17 (47.2) | 17 (45.9) | 20 (57.1) | 11 (31.4) | 65 (45.5) |
| ≥25-<30 | 10 (27.8) | 10 (27.0) | 6 (17.1) | 18 (51.4) | 44 (30.8) |
| ≥30 | 9 (25.0) | 10 (27.0) | 9 (25.7) | 6 (17.1) | 34 (23.8) |

BMI: Body mass index
^{a}Asia: South Korea, Taiwan, Japan; Latin America: Argentina, Chile; Eastern Europe: Poland; Western Countries: Spain, Italy, Germany, Canada, Greece, Netherlands.

TABLE 11
Demographics and Participant Characteristics at Baseline (Patients with Prior
Omalizumab Use, Including Omalizumab-Incomplete Responders in ITT Population)

| | | | | |
|---|---|---|---|---|
| | Rilzabrutinib | Rilzabrutinib | Rilzabrutinib | |
| Placebo | 400 mg QPM | 400 mg BID | 400 mg TID | All |
| (N = 4) | (N = 1) | (N = 6) | (N = 6) | (N = 17) |

Age (years)

| | | | | | |
|---|---|---|---|---|---|
| Number | 4 | 1 | 6 | 6 | 17 |
| Mean (SD) | 48.0 (16.5) | 48.0 (NC) | 45.7 (12.8) | 47.0 (8.9) | 46.8 (11.3) |
| Median | 53.0 | 48.0 | 41.5 | 50.0 | 47.0 |
| Q1; Q3 | 36.0; 60.0 | 48.0; 48.0 | 34.0; 59.0 | 39.0; 54.0 | 39.0; 55.0 |
| Min; Max | 25; 61 | 48; 48 | 34; 64 | 34; 55 | 25; 64 |

Age group (years) [n (%)]

| | | | | | |
|---|---|---|---|---|---|
| Number | 4 | 1 | 6 | 6 | 17 |
| 18-39 | 1 (25.0) | 0 | 2 (33.3) | 2 (33.3) | 5 (29.4) |
| 40-64 | 3 (75.0) | 1 (100) | 4 (66.7) | 4 (66.7) | 12 (70.6) |
| ≥65 | 0 | 0 | 0 | 0 | 0 |

Age group (years) [n (%)]

| | | | | | |
|---|---|---|---|---|---|
| Number | 4 | 1 | 6 | 6 | 17 |
| <44 (Median) | 1 (25.0) | 0 | 4 (66.7) | 2 (33.3) | 7 (41.2) |
| ≥44 (Median) | 3 (75.0) | 1 (100) | 2 (33.3) | 4 (66.7) | 10 (58.8) |

Region^{a} [n (%)]

| | | | | | |
|---|---|---|---|---|---|
| Number | 4 | 1 | 6 | 6 | 17 |
| Latin America | 1 (25.0) | 0 | 1 (16.7) | 1 (16.7) | 3 (17.6) |
| Eastern Europe | 0 | 0 | 0 | 1 (16.7) | 1 (5.9) |
| Western Countries | 3 (75.0) | 1 (100) | 5 (83.3) | 4 (66.7) | 13 (76.5) |

Sex [n (%)]

| | | | | | |
|---|---|---|---|---|---|
| Number | 4 | 1 | 6 | 6 | 17 |
| Male | 1 (25.0) | 1 (100) | 3 (50.0) | 1 (16.7) | 6 (35.3) |
| Female | 3 (75.0) | 0 | 3 (50.0) | 5 (83.3) | 11 (64.7) |

Race [n (%)]

| | | | | | |
|---|---|---|---|---|---|
| Number | 4 | 1 | 6 | 6 | 17 |
| White | 4 (100) | 1 (100) | 6 (100) | 5 (83.3) | 16 (94.1) |
| Black or African American | 0 | 0 | 0 | 0 | 0 |
| Asian | 0 | 0 | 0 | 0 | 0 |
| Native Hawaiian or Other Pacific | 0 | 0 | 0 | 0 | 0 |
| Islander | | | | | |
| American Indian or Alaska Native | 0 | 0 | 0 | 1 (16.7) | 1 (5.9) |
| Multiple | 0 | 0 | 0 | 0 | 0 |
| Not reported | 0 | 0 | 0 | 0 | 0 |
| Unknown | 0 | 0 | 0 | 0 | 0 |

Ethnicity [n (%)]

| | | | | | |
|---|---|---|---|---|---|
| Number | 4 | 1 | 6 | 6 | 17 |
| Hispanic or Latino | 1 (25.0) | 1 (100) | 1 (16.7) | 1 (16.7) | 4 (23.5) |
| Not Hispanic or Latino | 3 (75.0) | 0 | 5 (83.3) | 5 (83.3) | 13 (76.5) |
| Not reported | 0 | 0 | 0 | 0 | 0 |
| Unknown | 0 | 0 | 0 | 0 | 0 |

Weight (kg)

| | | | | | |
|---|---|---|---|---|---|
| Number | 4 | 1 | 6 | 6 | 17 |
| Mean (SD) | 88.55 (18.49) | 60.10 (NC) | 84.73 (8.01) | 85.63 (20.59) | 84.50 (16.08) |
| Median | 89.85 | 60.10 | 88.00 | 76.00 | 84.00 |
| Q1; Q3 | 72.85; 104.25 | 60.10; 60.10 | 84.00; 89.40 | 75.00; 108.00 | 75.00; 90.00 |
| Min; Max | 68.7; 105.8 | 60.1; 60.1 | 69.0; 90.0 | 64.0; 114.8 | 60.1; 114.8 |

Weight group (kg) [n (%)]

| | | | | | |
|---|---|---|---|---|---|
| Number | 4 | 1 | 6 | 6 | 17 |
| <72.9 (Median) | 1 (25.0) | 1 (100) | 1 (16.7) | 1 (16.7) | 4 (23.5) |
| ≥72.9 (Median) | 3 (75.0) | 0 | 5 (83.3) | 5 (83.3) | 13 (76.5) |

Weight group (kg) [n (%)]

| | | | | | |
|---|---|---|---|---|---|
| Number | 4 | 1 | 6 | 6 | 17 |
| <60 | 0 | 0 | 0 | 0 | 0 |
| ≥60-<90 | 2 (50.0) | 1 (100) | 5 (83.3) | 4 (66.7) | 12 (70.6) |
| ≥90 | 2 (50.0) | 0 | 1 (16.7) | 2 (33.3) | 5 (29.4) |

BMI (kg/m²)

| | | | | | |
|---|---|---|---|---|---|
| Number | 4 | 1 | 6 | 6 | 17 |
| Mean (SD) | 32.05 (7.69) | 18.50 (NC) | 30.03 (6.54) | 30.60 (7.09) | 30.03 (7.05) |
| Median | 30.35 | 18.50 | 28.70 | 28.60 | 28.70 |
| Q1; Q3 | 26.30; 37.80 | 18.50; 18.50 | 26.90; 30.10 | 25.00; 35.70 | 25.10; 33.20 |
| Min; Max | 25.1; 42.4 | 18.5; 18.5 | 23.3; 42.5 | 23.5; 42.2 | 18.5; 42.5 |

BMI group (kg/m²) [n (%)]

| | | | | | |
|---|---|---|---|---|---|
| Number | 4 | 1 | 6 | 6 | 17 |
| <25 | 0 | 1 (100) | 1 (16.7) | 1 (16.7) | 3 (17.6) |
| ≥25-<30 | 2 (50.0) | 0 | 3 (50.0) | 3 (50.0) | 8 (47.1) |
| ≥30 | 2 (50.0) | 0 | 2 (33.3) | 2 (33.3) | 6 (35.3) |

BMI: Body mass index
^{a}Asia: South Korea, Taiwan, Japan; Latin America: Argentina, Chile; Eastern Europe: Poland; Western Countries: Spain, Italy, Germany, Canada, Greece, Netherlands.

TABLE 12
Disease and Other Characteristics at Baseline (Randomized Population)

| | | | | |
|---|---|---|---|---|
| | Rilzabrutinib | Rilzabrutinib | Rilzabrutinib | |
| Placebo | 400 mg QPM | 400 mg BID | 400 mg TID | All |
| (N = 40) | (N = 38) | (N = 41) | (N = 41) | (N = 160) |

Age at onset of CSU (years)

| | | | | | |
|---|---|---|---|---|---|
| Number | 40 | 38 | 41 | 41 | 160 |
| Mean (SD) | 34.7 (12.2) | 37.3 (14.1) | 41.9 (15.3) | 42.0 (13.4) | 39.0 (14.0) |
| Median | 31.5 | 38.0 | 41.0 | 42.0 | 38.0 |
| Q1; Q3 | 25.0; 43.5 | 25.0; 49.0 | 31.0; 54.0 | 33.0; 49.0 | 28.0; 49.0 |
| Min; Max | 17; 62 | 12; 65 | 2; 71 | 8; 70 | 2; 71 |

Time since first diagnosis of CSU (years)^{a}

| | | | | | |
|---|---|---|---|---|---|
| Number | 40 | 38 | 41 | 41 | 160 |
| Mean (SD) | 6.13 (8.89) | 5.26 (5.56) | 4.11 (6.96) | 6.99 (11.37) | 5.63 (8.51) |
| Median | 3.50 | 3.63 | 2.00 | 3.08 | 3.00 |
| Q1; Q3 | 1.21; 6.79 | 1.17; 6.00 | 1.08; 4.17 | 1.08; 7.25 | 1.08; 6.00 |
| Min; Max | 0.3; 39.0 | 0.6; 24.0 | 0.3; 41.0 | 0.3; 53.0 | 0.3; 53.0 |

Prior use of Omalizumab [n (%)]

| | | | | | |
|---|---|---|---|---|---|
| Number | 40 | 38 | 41 | 41 | 160 |
| Yes | 4 (10.0) | 1 (2.6) | 6 (14.6) | 6 (14.6) | 17 (10.6) |
| No | 36 (90.0) | 37 (97.4) | 35 (85.4) | 35 (85.4) | 143 (89.4) |

History of allergy^{b}

| | | | | | |
|---|---|---|---|---|---|
| Number | 40 | 38 | 41 | 41 | 160 |
| Allergic | 6 (15.0) | 10 (26.3) | 13 (31.7) | 12 (29.3) | 41 (25.6) |
| Non-Allergic | 34 (85.0) | 28 (73.7) | 28 (68.3) | 29 (70.7) | 119 (74.4) |

History of atopic dermatitis

| | | | | | |
|---|---|---|---|---|---|
| Number | 40 | 38 | 41 | 41 | 160 |
| Yes | 1 (2.5) | 0 | 1 (2.4) | 1 (2.4) | 3 (1.9) |
| No | 39 (97.5) | 38 (100) | 40 (97.6) | 40 (97.6) | 157 (98.1) |

History of angioedema

| | | | | | |
|---|---|---|---|---|---|
| Number | 40 | 38 | 41 | 41 | 160 |
| Yes | 18 (45.0) | 16 (42.1) | 12 (29.3) | 13 (31.7) | 59 (36.9) |
| No | 22 (55.0) | 22 (57.9) | 29 (70.7) | 28 (68.3) | 101 (63.1) |

Number of angioedema episodes in past 6 months

| | | | | | |
|---|---|---|---|---|---|
| Number | 18 | 17 | 12 | 13 | 60 |
| Mean (SD) | 9.5 (14.6) | 8.6 (14.4) | 11.4 (20.3) | 14.5 (16.0) | 10.7 (15.9) |
| Median | 4.0 | 1.0 | 1.5 | 6.0 | 2.5 |
| Q1; Q3 | 1.0; 10.0 | 0.0; 12.0 | 1.0; 17.5 | 3.0; 30.0 | 1.0; 16.5 |
| Min; Max | 0; 60 | 0; 48 | 0; 70 | 0; 48 | 0; 70 |

Time since last angioedema episode (months)

| | | | | | |
|---|---|---|---|---|---|
| Number | 18 | 16 | 12 | 13 | 59 |
| Mean (SD) | 7.14 (21.22) | 6.62 (11.64) | 21.31 (64.64) | 10.39 (29.51) | 10.60 (34.22) |
| Median | 1.41 | 2.83 | 1.81 | 1.87 | 1.58 |
| Q1; Q3 | 0.92; 3.19 | 0.71; 8.20 | 0.76; 4.63 | 1.02; 3.29 | 0.76; 4.50 |
| Min; Max | 0.5; 91.8 | 0.4; 48.2 | 0.5; 226.4 | 0.2; 108.4 | 0.2; 226.4 |

Baseline UAS7 score

| | | | | | |
|---|---|---|---|---|---|
| Number | 40 | 38 | 41 | 41 | 160 |
| Mean (SD) | 30.0 (8.6) | 31.4 (7.3) | 30.2 (7.2) | 29.9 (8.6) | 30.3 (7.9) |
| Median | 29.0 | 30.0 | 28.0 | 29.0 | 29.0 |
| Q1; Q3 | 23.0; 37.5 | 27.0; 38.0 | 25.0; 35.0 | 25.0; 36.0 | 25.0; 37.0 |
| Min; Max | 16; 42 | 16; 42 | 18; 42 | 0; 42 | 0; 42 |

Baseline UAS7 score [n (%)]

| | | | | | |
|---|---|---|---|---|---|
| Number | 40 | 38 | 41 | 41 | 160 |
| <28 | 15 (37.5) | 10 (26.3) | 16 (39.0) | 14 (34.1) | 55 (34.4) |
| ≥28 | 25 (62.5) | 28 (73.7) | 25 (61.0) | 27 (65.9) | 105 (65.6) |

Baseline ISS7 score

| | | | | | |
|---|---|---|---|---|---|
| Number | 40 | 38 | 41 | 41 | 160 |
| Mean (SD) | 15.6 (4.2) | 16.5 (3.5) | 15.8 (3.7) | 15.9 (4.4) | 15.9 (4.0) |
| Median | 14.5 | 16.5 | 15.0 | 15.0 | 15.0 |
| Q1; Q3 | 12.0; 20.5 | 14.0; 20.0 | 13.0; 19.0 | 14.0; 20.0 | 14.0; 20.0 |
| Min; Max | 8; 21 | 9; 21 | 8; 21 | 0; 21 | 0; 21 |

Baseline ISS7 score [n (%)]

| | | | | | |
|---|---|---|---|---|---|
| Number | 40 | 38 | 41 | 41 | 160 |
| <13 | 11 (27.5) | 5 (13.2) | 7 (17.1) | 4 (9.8) | 27 (16.9) |
| ≥13 | 29 (72.5) | 33 (86.8) | 34 (82.9) | 37 (90.2) | 133 (83.1) |

Baseline HSS7 score

| | | | | | |
|---|---|---|---|---|---|
| Number | 40 | 38 | 41 | 41 | 160 |
| Mean (SD) | 14.4 (4.9) | 14.9 (4.3) | 14.4 (4.3) | 14.0 (4.8) | 14.4 (4.5) |
| Median | 14.0 | 14.0 | 14.0 | 14.0 | 14.0 |
| Q1; Q3 | 11.0; 18.9 | 12.0; 18.0 | 12.0; 17.0 | 12.0; 17.0 | 12.0; 18.0 |
| Min; Max | 7; 21 | 7; 21 | 7; 21 | 0; 21 | 0; 21 |

Baseline AAS7 >0 [n (%)]

| | | | | | |
|---|---|---|---|---|---|
| Number | 40 | 38 | 41 | 41 | 160 |
| Yes | 30 (75.0) | 28 (73.7) | 28 (68.3) | 26 (63.4) | 112 (70.0) |
| No | 10 (25.0) | 10 (26.3) | 13 (31.7) | 15 (36.6) | 48 (30.0) |

Baseline AAS7 score for participants with angioedema^{c}

| | | | | | |
|---|---|---|---|---|---|
| Number | 30 | 28 | 28 | 26 | 112 |
| Mean (SD) | 59.7 (27.1) | 54.0 (25.2) | 46.1 (25.7) | 46.0 (22.3) | 51.7 (25.6) |
| Median | 61.5 | 56.0 | 44.5 | 50.5 | 51.0 |
| Q1; Q3 | 42.0; 86.0 | 39.0; 71.0 | 24.0; 67.5 | 27.0; 61.0 | 32.0; 68.0 |
| Min; Max | 8; 101 | 6; 99 | 2; 92 | 9; 91 | 2; 101 |

Baseline UCT score

| | | | | | |
|---|---|---|---|---|---|
| Number | 35 | 36 | 41 | 40 | 152 |
| Mean (SD) | 4.2 (3.1) | 4.0 (2.2) | 4.2 (3.1) | 4.5 (3.5) | 4.3 (3.0) |
| Median | 4.0 | 4.0 | 4.0 | 4.0 | 4.0 |
| Q1; Q3 | 2.0; 6.0 | 2.0; 5.5 | 2.0; 6.0 | 2.0; 6.5 | 2.0; 6.0 |
| Min; Max | 0; 12 | 0; 9 | 0; 13 | 0; 15 | 0; 15 |

Baseline PGIS score

| | | | | | |
|---|---|---|---|---|---|
| Number | 40 | 38 | 41 | 40 | 159 |
| Mean (SD) | 2.4 (0.6) | 2.5 (0.6) | 2.2 (0.6) | 2.4 (0.7) | 2.4 (0.6) |
| Median | 2.0 | 3.0 | 2.0 | 2.0 | 2.0 |
| Q1; Q3 | 2.0; 3.0 | 2.0; 3.0 | 2.0; 3.0 | 2.0; 3.0 | 2.0; 3.0 |
| Min; Max | 1; 3 | 1; 3 | 1; 3 | 0; 3 | 0; 3 |

Baseline Eosinophils (10\hat{ }9/L)

| | | | | | |
|---|---|---|---|---|---|
| Number | 40 | 38 | 41 | 41 | 160 |
| Mean (SD) | 0.194 (0.104) | 0.189 (0.134) | 0.192 (0.150) | 0.200 (0.144) | 0.194 (0.133) |
| Median | 0.180 | 0.155 | 0.160 | 0.180 | 0.165 |
| Q1; Q3 | 0.115; 0.290 | 0.110; 0.250 | 0.090; 0.260 | 0.090; 0.290 | 0.090; 0.280 |
| Min; Max | 0.03; 0.45 | 0.02; 0.67 | 0.02; 0.62 | 0.02; 0.64 | 0.02; 0.67 |

Baseline Eosinophils (10\hat{ }9/L) [n (%)]

| | | | | | |
|---|---|---|---|---|---|
| Number | 40 | 38 | 41 | 41 | 160 |
| <0.165 (Median) | 19 (47.5) | 20 (52.6) | 22 (53.7) | 19 (46.3) | 80 (50.0) |
| ≥0.165 (Median) | 21 (52.5) | 18 (47.4) | 19 (46.3) | 22 (53.7) | 80 (50.0) |

Baseline Eosinophils (10\hat{ }9/L) [n (%)]

| | | | | | |
|---|---|---|---|---|---|
| Number | 40 | 38 | 41 | 41 | 160 |
| <0.3 | 31 (77.5) | 33 (86.8) | 33 (80.5) | 32 (78.0) | 129 (80.6) |
| ≥0.3 | 9 (22.5) | 5 (13.2) | 8 (19.5) | 9 (22.0) | 31 (19.4) |

Baseline Basophils (10\hat{ }9/L)

| | | | | | |
|---|---|---|---|---|---|
| Number | 40 | 38 | 41 | 41 | 160 |
| Mean (SD) | N/A | N/A | N/A | N/A | N/A |
| Median | 0.040 | 0.040 | 0.040 | 0.040 | 0.040 |
| Q1; Q3 | (0.023; 0.050) | (0.015; 0.070) | (0.015; 0.060) | (0.030; 0.050) | (0.015; 0.060) |
| Min; Max | N/A | N/A | N/A | N/A | N/A |

Baseline Total IgE (IU/mL)

| | | | | | |
|---|---|---|---|---|---|
| Number | 39 | 37 | 41 | 40 | 157 |
| Mean (SD) | 319.6 (530.2) | 433.3 (669.8) | 275.7 (487.3) | 389.0 (638.7) | 352.6 (581.9) |
| Median | 142.6 | 158.0 | 111.1 | 116.8 | 123.8 |
| Q1; Q3 | 40.4; 349.5 | 37.6; 533.6 | 38.7; 314.6 | 34.7; 407.9 | 37.6; 351.1 |
| Min; Max | 0; 2608 | 3; 2843 | 1; 2634 | 1; 2882 | 0; 2882 |

Baseline Total IgE (IU/mL) [n (%)]

| | | | | | |
|---|---|---|---|---|---|
| Number | 39 | 37 | 41 | 40 | 157 |
| <300 | 27 (69.2) | 25 (67.6) | 29 (70.7) | 28 (70.0) | 109 (69.4) |
| ≥300 | 12 (30.8) | 12 (32.4) | 12 (29.3) | 12 (30.0) | 48 (30.6) |

Baseline Total IgE (IU/mL) [n (%)]

| | | | | | |
|---|---|---|---|---|---|
| Number | 39 | 37 | 41 | 40 | 157 |
| <100 | 18 (46.2) | 15 (40.5) | 19 (46.3) | 18 (45.0) | 70 (44.6) |
| ≥100 | 21 (53.8) | 22 (59.5) | 22 (53.7) | 22 (55.0) | 87 (55.4) |

Baseline Total IgG (g/L)

| | | | | | |
|---|---|---|---|---|---|
| Number | 40 | 38 | 41 | 41 | 160 |
| Mean (SD) | 12.4 (3.2) | 11.6 (2.0) | 11.3 (2.3) | 11.7 (2.1) | 11.7 (2.5) |
| Median | 11.9 | 11.1 | 11.4 | 11.7 | 11.5 |
| Q1; Q3 | 10.2; 14.0 | 9.9; 13.0 | 10.2; 12.4 | 10.4; 12.9 | 10.1; 13.0 |
| Min; Max | 6; 24 | 8; 17 | 6; 19 | 7; 18 | 6; 24 |

Baseline Total IgG (g/L) [n (%)]

| | | | | | |
|---|---|---|---|---|---|
| Number | 40 | 38 | 41 | 41 | 160 |
| <7 | 1 (2.5) | 0 | 1 (2.4) | 0 | 2 (1.3) |
| 7-16 | 33 (82.5) | 37 (97.4) | 39 (95.1) | 40 (97.6) | 149 (93.1) |
| >16 | 6 (15.0) | 1 (2.6) | 1 (2.4) | 1 (2.4) | 9 (5.6) |

Baseline Total IgM (g/L)

| | | | | | |
|---|---|---|---|---|---|
| Number | 39 | 37 | 41 | 41 | 158 |
| Mean (SD) | 1.1 (0.6) | 1.1 (0.5) | 1.2 (0.7) | 1.3 (0.8) | 1.2 (0.6) |
| Median | 1.0 | 1.1 | 1.1 | 1.1 | 1.1 |
| Q1; Q3 | 0.7; 1.3 | 0.7; 1.5 | 0.7; 1.4 | 0.7; 1.6 | 0.7; 1.4 |
| Min; Max | 0; 3 | 0; 2 | 0; 4 | 0; 4 | 0; 4 |

Baseline Total IgM (g/L) [n (%)]

| | | | | | |
|---|---|---|---|---|---|
| Number | 39 | 37 | 41 | 41 | 158 |
| <0.4 | 0 | 2 (5.4) | 1 (2.4) | 1 (2.4) | 4 (2.5) |
| 0.4-2.3 | 36 (92.3) | 35 (94.6) | 38 (92.7) | 34 (82.9) | 143 (90.5) |
| >2.3 | 3 (7.7) | 0 | 2 (4.9) | 6 (14.6) | 11 (7.0) |

Baseline IgG anti-IgE (Arbitrary U)

| | | | | | |
|---|---|---|---|---|---|
| Number | 40 | 38 | 41 | 41 | 160 |
| Mean (SD) | N/A | N/A | N/A | N/A | N/A |
| Median | 5982.6 | 6983.4 | 6911.4 | 6230.8 | 6437.3 |
| Q1; Q3 | (3954.8; | (3807.8; | (5329.0; | (4608.6; | (4608.6; |
| | 9595.8) | 11618.6) | 10065.8) | 9534.6) | 10147.6) |
| Min; Max | N/A | N/A | N/A | N/A | N/A |

Baseline BHRA [n (%)]

| | | | | | |
|---|---|---|---|---|---|
| Number | 40 | 36 | 41 | 41 | 158 |
| BHRA+ | 5 (12.5) | 4 (11.1) | 7 (17.1) | 6 (14.6) | 22 (13.9) |
| BHRA− | 35 (87.5) | 32 (88.9) | 34 (82.9) | 35 (85.4) | 136 (86.1) |

Baseline IgG anti-FceRI (Arbitrary U)

| | | | | | |
|---|---|---|---|---|---|
| Number | 40 | 38 | 41 | 41 | 160 |
| Mean (SD) | 312.3 (530.0) | 931.6 (4512.2) | 1591.8 (7225.6) | 214.9 (298.3) | 762.3 (4275.0) |
| Median | 141.6 | 174.7 | 208.2 | 134.2 | 160.1 |
| Q1; Q3 | 56.1; 308.9 | 53.7; 250.0 | 99.8; 425.0 | 46.4; 308.9 | 53.5; 317.6 |
| Min; Max | 0; 2762 | 0; 27990 | 0; 46369 | 0; 1695 | 0; 46369 |

Baseline IgE anti-TPO (Arbitrary U)

| | | | | | |
|---|---|---|---|---|---|
| Number | 40 | 38 | 41 | 41 | 160 |
| Mean (SD) | 2.7 (0.6) | 2.9 (1.3) | 2.7 (0.5) | 2.8 (0.7) | 2.8 (0.8) |
| Median | 2.5 | 2.5 | 2.6 | 2.6 | 2.6 |
| Q1; Q3 | 2.2; 3.2 | 2.4; 3.2 | 2.4; 2.9 | 2.2; 3.0 | 2.3; 3.1 |
| Min; Max | 2; 4 | 2; 10 | 2; 4 | 2; 5 | 2; 10 |

Baseline IL-31

| | | | | | |
|---|---|---|---|---|---|
| Number | 40 | 38 | 41 | 41 | 160 |
| Median | 4.0 | 4.1 | 5.0 | 4.2 | 4.2 |
| Q1; Q3 | (2.9; 5.3) | (3.2; 5.7) | (3.8; 8.4) | (3.3; 6.7) | (3.3; 6.4) |

Baseline sMRGPRX2 (ng/mL)

| Number | 40 | 38 | 41 | 41 | 160 |
|---|---|---|---|---|---|
| Median | 21.1 | 26.5 | 17.5 | 21.8 | 21.2 |
| Q1; Q3 | (13.1; 30.6) | (17.5; 33.6) | (10.4; 28.3) | (14.3: 30.3) | (13.6; 31.2) |
| Baseline IgG anti-TPO (IU) | | | | | |
| Number | 40 | 38 | 40 | 41 | 159 |
| Mean (SD) | 18.7 (50.0) | 35.4 (107.4) | 47.7 (182.1) | 36.1 (80.7) | 34.5 (115.2) |
| Median | 0.0 | 0.0 | 3.1 | 1.6 | 0.4 |
| Q1; Q3 | 0.0; 6.8 | 0.0; 8.3 | 0.0; 9.7 | 0.0; 19.9 | 0.0; 10.3 |
| Min; Max | 0; 205 | 0; 429 | 0; 1133 | 0; 338 | 0; 1133 |

Baseline H1-antihistamines dose [n (%)]

| | | | | | |
|---|---|---|---|---|---|
| Number | 40 | 38 | 41 | 40 | 159 |
| <1-fold | 1 (2.5) | 0 | 0 | 0 | 1 (0.6) |
| 1-fold | 22 (55.0) | 21 (55.3) | 16 (39.0) | 24 (60.0) | 83 (52.2) |
| 2-fold to 3-fold | 9 (22.5) | 14 (36.8) | 14 (34.1) | 9 (22.5) | 46 (28.9) |
| 4-fold | 8 (20.0) | 3 (7.9) | 11 (26.8) | 7 (17.5) | 29 (18.2) |

Frequency of alcohol drinking in the past 12 months [n (%)]

| | | | | | |
|---|---|---|---|---|---|
| Number | 40 | 38 | 41 | 41 | 160 |
| Never | 19 (47.5) | 17 (44.7) | 19 (46.3) | 22 (53.7) | 77 (48.1) |
| Occasional | 16 (40.0) | 15 (39.5) | 18 (43.9) | 16 (39.0) | 65 (40.6) |
| At least monthly | 1 (2.5) | 0 | 2 (4.9) | 0 | 3 (1.9) |
| At least weekly | 2 (5.0) | 5 (13.2) | 1 (2.4) | 2 (4.9) | 10 (6.3) |
| At least daily | 2 (5.0) | 1 (2.6) | 1 (2.4) | 1 (2.4) | 5 (3.1) |

Number of drinks on a typical day of alcohol drinking in the past 12 months [n (%)]

| | | | | | |
|---|---|---|---|---|---|
| Number | 21 | 21 | 22 | 19 | 83 |
| 1 to 3 | 21 (100) | 21 (100) | 21 (95.5) | 19 (100) | 82 (98.8) |
| >3 | 0 | 0 | 1 (4.5) | 0 | 1 (1.2) |

^{a}Derived as (Year of randomization − Year of first diagnosis of CSU) + (month of randomization-month of first diagnosis of CSU)/12
^{b}Defined as having a medical history of atopic dermatitis, allergic rhinitis, allergic conjunctivitis, asthma, food allergy, chronic rhinosinusitis, nasal polyps or eosinophilic esophagitis.
^{c}Participants with an AAS7 baseline score >0.
Note:
A low score indicates good outcome for ISS7 (range 0-21), UAS7 (range 0-42), HSS7 (range 0-21), AAS7 (0-105), UCT (range 0-16), and PGIS (range 1-4).

### UAS7

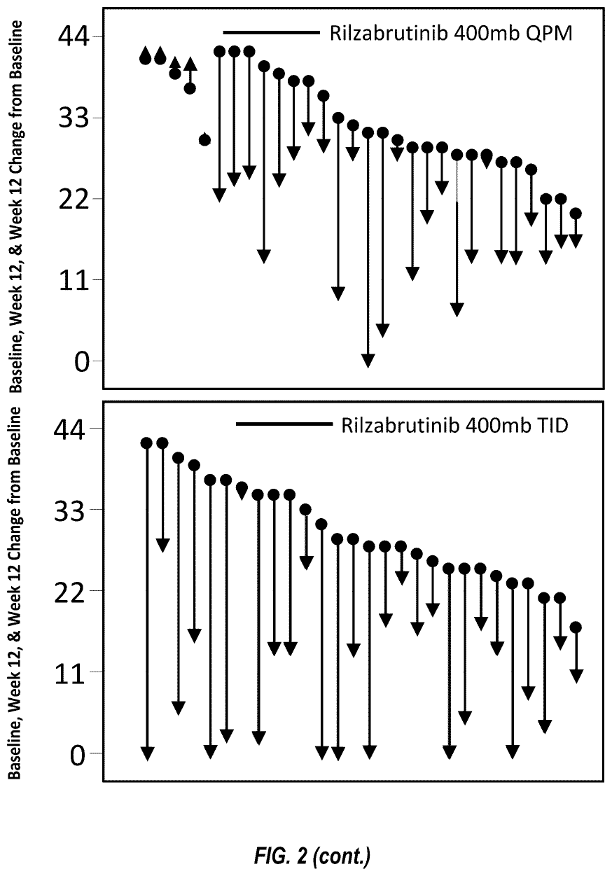

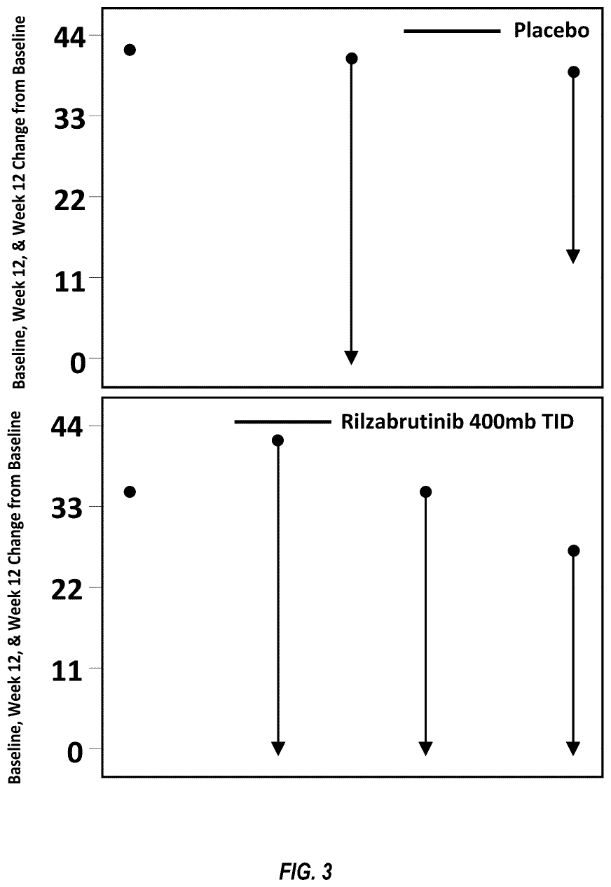

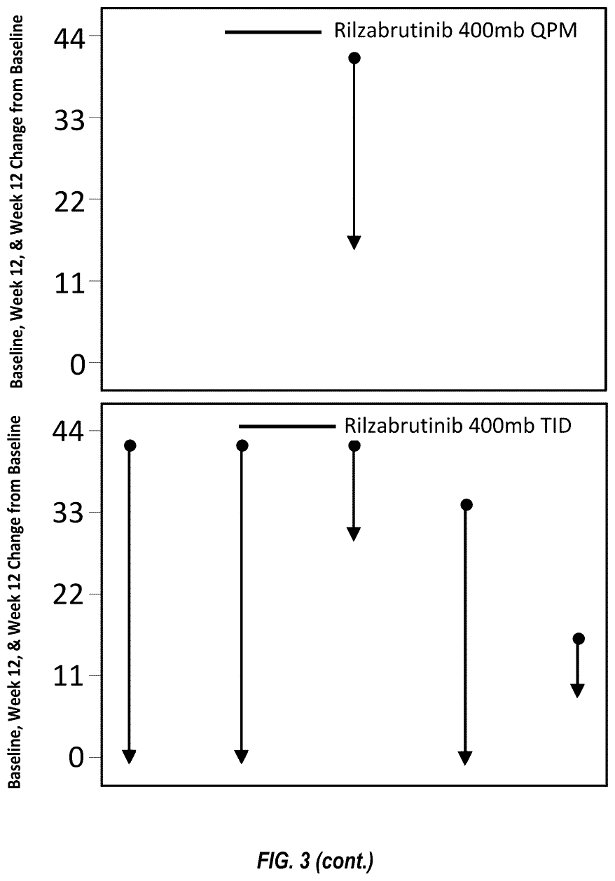

Treatment with rilzabrutinib yielded significant improvements in UAS7. In particular, patients receiving 400 mg TID rilzabrutinib saw a significant reduction in UAS7 at Week 12, thereby meeting the primary endpoint in the non-United States countries (see FIGS. 2-4 and Tables 13-16). In the ITT population, participants receiving 400 mg TID rilzabrutinib showed a LS mean change from baseline at Week 12 of −17.95 (p=0.00116) points, while patients receiving the placebo showed a LS mean change of −11.20 from baseline to Week 12 (see Table 13). Consistent results were observed for omalizumab-naïve patients receiving 400 mg TID rilzabrutinib, both prior to (LS mean change of −16.89, p=0.0159; see Table 14) and following (LS mean reduction of −17.50, p=0.0079; see Table 15) the removal of a non-symptomatic outlier (UAS7/ISS7=0 at baseline) who was erroneously randomized into the 400 mg TID rilzabrutinib arm.

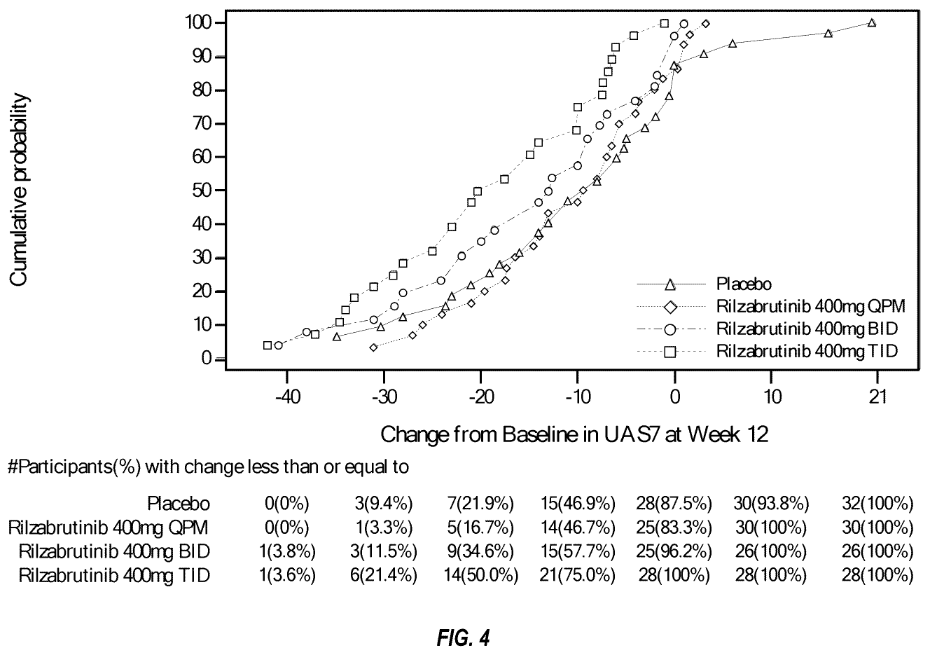

In the 400 mg TID arm, all patients except for one showed improvement in the weekly UAS7. Moreover, a clear separation was observed between patients receiving 400 mg TID rilzabrutinib and patients receiving the placebo across all response thresholds (see FIG. 4).

Subgroup analysis of the omalizumab-naïve population further supports the efficacy of rilzabrutinib treatment in CSU (see Table 16). Overall, treatment with 400 mg TID rilzabrutinib was efficacious across subgroups, regardless of baseline demographic characteristics. Moreover, 400 mg TID rilzabrutinib treatment led to improvements in patients with a low IgE concentration, positive BHRA status, history of angioedema, or characteristics of difficult-to-treat CSU.

TABLE 13
Supplementary Analysis: Change from Baseline in UAS7 at Week 12 (ITT Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| UAS7 | (N = 40) | (N = 38) | (N = 41) | (N = 41) |

Baseline

| | | | | |
|---|---|---|---|---|
| Number | 40 | 38 | 41 | 41 |
| Mean (SD) | 29.97 (8.56) | 31.37 (7.32) | 30.21 (7.19) | 29.88 (8.57) |
| Median | 29.00 | 30.00 | 28.00 | 29.00 |
| Q1; Q3 | 23.00; 37.50 | 27.00; 38.00 | 25.00; 35.00 | 25.00; 36.00 |
| Min; Max | 16.0; 42.0 | 16.0; 42.0 | 18.0; 42.0 | 0.0; 42.0 |

Week 12

| | | | | |
|---|---|---|---|---|
| Number (observed/imputed) | 35 (33/2) | 31 (28/3) | 30 (27/3) | 33 (30/3) |
| Mean (SD) | 19.27 (13.49) | 21.80 (10.94) | 14.70 (14.29) | 10.52 (10.26) |
| Median | 17.50 | 22.40 | 12.00 | 9.00 |
| Q1; Q3 | 7.00; 29.00 | 14.00; 28.00 | 0.00; 26.00 | 0.00; 16.80 |
| Min; Max | 0.0; 42.0 | 0.0; 42.0 | 0.0; 42.0 | 0.0; 35.0 |

Change from baseline

| | | | | |
|---|---|---|---|---|
| Number (observed/imputed) | 35 (33/2) | 31 (28/3) | 30 (27/3) | 33 (30/3) |
| Mean (SD) | −10.76 (13.99) | −11.04 (9.56) | −16.02 (13.29) | −20.30 (12.41) |
| Median | −8.00 | −9.40 | −13.50 | −20.33 |
| Q1; Q3 | −21.00; −0.50 | −17.50; −3.67 | −27.00; −4.00 | −31.00; −10.00 |
| Min; Max | −40.8; 20.5 | −31.0; 3.3 | −42.0; 1.0 | −42.0; −1.0 |
| LS Mean (SE) ^{a} | −11.20 (1.98) | −10.22 (2.01) | −14.82 (1.98) | −17.95 (1.95) |
| LS Mean Diff vs. placebo (95% CI) ^{a} | | 0.97 (−4.40, 6.34) | −3.62 (−8.87, 1.63) | −6.76 (−12.01, −1.51) |
| P-value vs. placebo ^{a} | | 0.7230 | 0.1763 | 0.0116 |

^{a} Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.

TABLE 14
Primary Analysis: Change from Baseline in UAS7 at Week 12 (Omalizumab-Naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| UAS7 | (N = 36) | (N = 37) | (N = 35) | (N = 35) |

Baseline

| | | | | |
|---|---|---|---|---|
| Number | 36 | 37 | 35 | 35 |
| Mean (SD) | 28.97 (8.39) | 31.11 (7.24) | 29.73 (7.11) | 29.34 (8.08) |
| Median | 28.00 | 30.00 | 28.00 | 28.00 |
| Q1; Q3 | 22.00; 35.00 | 27.00; 38.00 | 24.50; 35.00 | 25.00; 35.00 |
| Min; Max | 16.0; 42.0 | 16.0; 42.0 | 18.0; 42.0 | 0.0; 42.0 |

Week 12

| | | | | |
|---|---|---|---|---|
| Number (observed/imputed) | 32 (31/1) | 30 (27/3) | 26 (24/2) | 28 (27/1) |
| Mean (SD) | 19.32 (13.04) | 22.00 (11.07) | 15.61 (13.91) | 11.00 (9.90) |
| Median | 17.75 | 22.87 | 14.00 | 12.25 |
| Q1; Q3 | 8.17; 28.50 | 14.00; 28.00 | 2.00; 26.00 | 1.00; 17.15 |
| Min; Max | 0.0; 42.0 | 0.0; 42.0 | 0.0; 42.0 | 0.0; 35.0 |

Change from baseline

| | | | | |
|---|---|---|---|---|
| Number (observed/imputed) | 32 (31/1) | 30 (27/3) | 26 (24/2) | 28 (27/1) |
| Mean (SD) | −9.71 (13.20) | −10.57 (9.36) | −14.49 (12.08) | −19.04 (11.40) |
| Median | −8.00 | −8.70 | −12.80 | −18.92 |
| Q1; Q3 | −18.50; −0.50 | −17.33; −3.67 | −22.00; −4.00 | −28.50; −8.75 |
| Min; Max | −35.0; 20.5 | −31.0; 3.3 | −40.8; 1.0 | −42.0; −1.0 |
| LS Mean (SE) ^{a} | −10.14 (2.05) | −9.74 (2.00) | −14.24 (2.06) | −16.89 (2.04) |
| LS Mean Diff vs. placebo (95% CI) ^{a} | | 0.40 (−5.08, 5.89) | −4.09 (−9.59, 1.40) | −6.75 (−12.23, −1.26) |
| P-value vs. placebo ^{a} | | 0.8856 | 0.1441 | 0.0159 |

^{a} Each of the imputed complete data were analyzed by fitting an ANCOVA model with the corresponding baseline value,
intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.

TABLE 15
Change from Baseline in UAS7 at Week 12 (Omalizumab-Naïve Population with Outlier Removed from Analysis)

| | | | | |
|---|---|---|---|---|
| | | Rilzabrutinib | Rilzabrutinib | Rilzabrutinib |
| | Placebo | 400 mg QPM | 400 mg BID | 400 mg TID |
| UAS7 | (N = 36) | (N = 37) | (N = 35) | (N = 34) |

Baseline

| | | | | |
|---|---|---|---|---|
| Number | 36 | 37 | 35 | 34 |
| Mean (SD) | 28.97 (8.39) | 31.11 (7.24) | 29.73 (7.11) | 30.21 (6.36) |
| Median | 28.00 | 30.00 | 28.00 | 28.50 |
| Q1; Q3 | 22.00; 35.00 | 27.00; 38.00 | 24.50; 35.00 | 25.00; 35.00 |
| Min; Max | 16.0; 42.0 | 16.0; 42.0 | 18.0; 42.0 | 17.0; 42.0 |

Week 12

| | | | | |
|---|---|---|---|---|
| Number (observed/imputed) | 32 (31/1) | 30 (27/3) | 26 (24/2) | 28 (27/1) |
| Mean (SD) | 19.32 (13.04) | 22.00 (11.07) | 15.61 (13.91) | 11.00 (9.90) |
| Median | 17.75 | 22.87 | 14.00 | 12.25 |
| Q1; Q3 | 8.17; 28.50 | 14.00; 28.00 | 2.00; 26.00 | 1.00; 17.15 |
| Min; Max | 0.0; 42.0 | 0.0; 42.0 | 0.0; 42.0 | 0.0; 35.0 |

Change from baseline

| | | | | |
|---|---|---|---|---|
| Number (observed/imputed) | 32 (31/1) | 30 (27/3) | 26 (24/2) | 28 (27/1) |
| Mean (SD) | −9.71 (13.20) | −10.57 (9.36) | −14.49 (12.08) | −19.04 (11.40) |
| Median | −8.00 | −8.70 | −12.80 | −18.92 |
| Q1; Q3 | −18.50; −0.50 | −17.33; −3.67 | −22.00; −4.00 | −28.50; −8.75 |
| Min; Max | −35.0; 20.5 | −31.0; 3.3 | −40.8; 1.0 | −42.0; −1.0 |
| LS Mean (SE) ^{a} | −10.01 (2.01) | −9.93 (1.96) | −14.30 (2.06) | −17.50 (2.06) |
| LS Mean Diff vs. placebo (95% CI) ^{a} | | 0.08 (−5.29, 5.46) | −4.29 (−9.74, 1.16) | −7.50 (−13.02, −1.97) |
| P-value vs. placebo ^{a} | | 0.9759 | 0.1232 | 0.0079 |

^{a} Each of the imputed complete data were analyzed by fitting an ANCOVA model with the corresponding baseline value, intervention group and region as covariates.

TABLE 16
Subgroup Analysis: Change from Baseline in UAS7 at Week 12 by Subgroups (Omalizumab-Naïve Population)

| | | | |
|---|---|---|---|
| | Rilzabrutinib | Rilzabrutinib | Rilzabrutinib |
| Placebo | 400 mg QPM | 400 mg BID | 400 mg TID |
| (N = 36) | (N = 37) | (N = 35) | (N = 35) |

Age group (years)

| | | | | |
|---|---|---|---|---|
| <44 (Median) | | | | |
| Number (observed/imputed) | 21 (20/1) | 16 (14/2) | 12 (12/0) | 8 (8/0) |
| Mean (SD) | −8.66 (13.45) | −10.45 (10.67) | −11.98 (9.69) | −18.69 (10.55) |
| LS Mean (SE) ^{a} | −8.39 (2.53) | −9.54 (2.74) | −13.80 (2.95) | −16.19 (3.68) |
| LS Mean Diff vs. placebo (95% CI) ^{a} | | −1.15 (−8.58, 6.27) | −5.42 (−13.05, 2.21) | −7.80 (−16.46, 0.86) |
| P-value vs. placebo ^{a} | | 0.7607 | 0.1642 | 0.0775 |
| ≥44 (Median) | | | | |
| Number (observed/imputed) | 11 (11/0) | 14 (13/1) | 14 (12/2) | 20 (19/1) |
| Mean (SD) | −11.71 (13.10) | −10.71 (7.99) | −16.63 (13.80) | −19.17 (11.98) |
| LS Mean (SE) ^{a} | −11.56 (3.28) | −8.84 (2.81) | −15.12 (2.72) | −17.54 (2.39) |
| LS Mean Diff vs. placebo (95% CI) ^{a} | | 2.72 (−5.76, 11.20) | −3.56 (−11.91, 4.80) | −5.98 (−13.92, 1.95) |
| P-value vs. placebo ^{a} | | 0.5296 | 0.4045 | 0.1393 |
| Overall p-value for interaction ^{b} | | | | 0.9595 |

Age group (years)

| | | | | |
|---|---|---|---|---|
| <65 | | | | |
| Number (observed/imputed) | 30 (29/1) | 29 (26/3) | 25 (23/2) | 24 (23/1) |
| Mean (SD) | −10.62 (12.74) | −10.37 (9.46) | −13.91 (11.95) | −18.19 (11.00) |
| LS Mean (SE) ^{a} | −10.41 (2.02) | −9.12 (1.96) | −13.99 (2.04) | −16.30 (2.11) |
| LS Mean Diff vs. placebo (95% CI) ^{a} | | 1.30 (−4.27, 6.86) | −3.58 (−9.19, 2.03) | −5.88 (−11.58, −0.19) |
| P-value vs. placebo ^{a} | | 0.6475 | 0.2106 | 0.0430 |
| ≥65 | | | | |
| Number (observed/imputed) | 2 (2/0) | 1 (1/0) | 1 (1/0) | 4 (4/0) |
| Mean (SD) | 4.00 (16.97) | −16.33 (NC) | −29.00 (NC) | −24.13 (14.17) |
| LS Mean (SE) ^{a} | 1.46 (10.15) | −1.73 (17.36) | −21.85 (10.98) | −23.81 (7.07) |
| LS Mean Diff vs. placebo (95% CI) ^{a} | | −3.19 (−44.28, 37.89) | −23.31 (−51.77, 5.15) | −25.27 (−49.57, −0.97) |
| P-value vs. placebo ^{a} | | 0.8790 | 0.1084 | 0.0416 |
| Overall p-value for interaction ^{b} | | | | 0.2251 |

Gender

| | | | | |
|---|---|---|---|---|
| Male | | | | |
| Number (observed/imputed) | 11 (11/0) | 8 (8/0) | 9 (8/1) | 5 (5/0) |
| Mean (SD) | −6.91 (13.75) | −9.58 (10.13) | −16.87 (13.20) | −12.72 (5.65) |
| LS Mean (SE) ^{a} | −6.70 (3.52) | −8.84 (4.15) | −16.67 (3.54) | −11.21 (4.99) |
| LS Mean Diff vs. placebo (95% CI) ^{a} | | −2.15 (−12.83, 8.54) | −9.97 (−19.70, −0.25) | −4.51 (−16.53, 7.51) |
| P-value vs. placebo ^{a} | | 0.6937 | 0.0445 | 0.4622 |
| Female | | | | |
| Number (observed/imputed) | 21 (20/1) | 22 (19/3) | 17 (16/1) | 23 (22/1) |
| Mean (SD) | −11.17 (12.99) | −10.93 (9.28) | −13.23 (11.66) | −20.41 (11.94) |
| LS Mean (SE) ^{a} | −11.39 (2.38) | −9.27 (2.22) | −12.97 (2.46) | −18.51 (2.16) |
| LS Mean Diff vs. placebo (95% CI) ^{a} | | 2.13 (−4.34, 8.59) | −1.57 (−8.27, 5.12) | −7.12 (−13.39, −0.85) |
| P-value vs. placebo ^{a} | | 0.5190 | 0.6450 | 0.0260 |
| Overall p-value for interaction ^{b} | | | | 0.3631 |

Baseline weight (kg)

| | | | | |
|---|---|---|---|---|
| <72.9 (Median) | | | | |
| Number (observed/imputed) | 18 (18/0) | 17 (16/1) | 16 (15/1) | 13 (13/0) |
| Mean (SD) | −11.18 (11.95) | −7.98 (7.20) | −12.97 (9.84) | −20.83 (13.25) |
| LS Mean (SE) ^{a} | −10.84 (2.51) | −7.76 (2.45) | −13.03 (2.58) | −18.62 (2.85) |
| LS Mean Diff vs. placebo (95% CI) ^{a} | | 3.08 (−3.85, 10.01) | −2.19 (−9.21, 4.84) | −7.78 (−15.19, −0.37) |
| P-value vs. placebo ^{a} | | 0.3833 | 0.5418 | 0.0397 |
| ≥72.9 (Median) | | | | |
| Number (observed/imputed) | 14 (13/1) | 13 (11/2) | 10 (9/1) | 15 (14/1) |
| Mean (SD) | −7.82 (14.89) | −13.96 (10.98) | −16.91 (15.27) | −17.48 (9.72) |
| LS Mean (SE) ^{a} | −7.93 (3.02) | −11.30 (3.07) | −15.67 (3.01) | −16.27 (2.71) |
| LS Mean Diff vs. placebo (95% CI) ^{a} | | −3.37 (−11.78, 5.05) | −7.75 (−16.17, 0.68) | −8.35 (−16.31, −0.38) |
| P-value vs. placebo ^{a} | | 0.4330 | 0.0715 | 0.0400 |
| Overall p-value for interaction ^{b} | | | | 0.4144 |

Baseline weight (kg)

| | | | | |
|---|---|---|---|---|
| <60 | | | | |
| Number (observed/imputed) | 10 (10/0) | 6 (5/1) | 7 (7/0) | 8 (8/0) |
| Mean (SD) | −8.75 (11.17) | −7.82 (8.01) | −10.00 (9.76) | −19.50 (14.84) |
| LS Mean (SE) ^{a} | −8.01 (3.53) | −6.74 (4.50) | −11.36 (3.92) | −19.90 (4.01) |
| LS Mean Diff vs. placebo (95% CI) ^{a} | | 1.28 (−9.97, 12.52) | −3.35 (−13.61, 6.91) | −11.88 (−22.36, −1.40) |
| P-value vs. placebo ^{a} | | 0.8237 | 0.5222 | 0.0262 |
| ≥60-<90 | | | | |
| Number (observed/imputed) | 15 (14/1) | 21 (19/2) | 16 (14/2) | 17 (17/0) |
| Mean (SD) | −10.84 (13.46) | −10.49 (9.74) | −15.04 (13.16) | −18.74 (10.60) |
| LS Mean (SE) ^{a} | −11.19 (2.81) | −9.11 (2.26) | −13.31 (2.65) | −15.74 (2.43) |
| LS Mean Diff vs. placebo (95% CI) ^{a} | | 2.08 (−5.04, 9.20) | −2.12 (−9.70, 5.46) | −4.55 (−11.84, 2.73) |
| P-value vs. placebo ^{a} | | 0.5666 | 0.5832 | 0.2205 |
| ≥90 | | | | |
| Number (observed/imputed) | 7 (7/0) | 3 (3/0) | 3 (3/0) | 3 (2/1) |
| Mean (SD) | −8.64 (16.85) | −16.67 (9.07) | −22.00 (9.00) | −19.44 (9.03) |
| LS Mean (SE) ^{a} | −8.78 (4.74) | −14.44 (7.38) | −20.74 (5.01) | −20.81 (7.30) |
| LS Mean Diff vs. placebo (95% CI) ^{a} | | −5.66 (−22.88, 11.55) | −11.96 (−25.48, 1.56) | −12.03 (−29.07, 5.01) |
| P-value vs. placebo ^{a} | | 0.5190 | 0.0829 | 0.1663 |
| Overall p-value for interaction ^{b} | | | | 0.7259 |

Baseline BMI (kg/m²)

| | | | | |
|---|---|---|---|---|
| <25 | | | | |
| Number (observed/imputed) | 15 (15/0) | 14 (13/1) | 17 (16/1) | 11 (11/0) |
| Mean (SD) | −8.97 (10.73) | −7.00 (7.22) | −14.44 (11.30) | −17.02 (13.10) |
| LS Mean (SE) ^{a} | −8.65 (2.64) | −6.27 (2.66) | −14.48 (2.43) | −17.23 (3.19) |
| LS Mean Diff vs. placebo (95% CI) ^{a} | | 2.37 (−4.98, 9.73) | −5.83 (−12.83, 1.16) | −8.58 (−16.69, −0.47) |
| P-value vs. placebo ^{a} | | 0.5266 | 0.1020 | 0.0381 |
| ≥25-<30 | | | | |
| Number (observed/imputed) | 9 (8/1) | 7 (6/1) | 5 (4/1) | 13 (13/0) |
| Mean (SD) | −5.13 (16.32) | −10.92 (12.01) | −13.03 (16.22) | −19.44 (10.56) |
| LS Mean (SE) ^{a} | −9.33 (3.97) | −7.48 (3.95) | −11.34 (4.80) | −14.99 (2.89) |
| LS Mean Diff vs. placebo (95% CI) ^{a} | | 1.85 (−9.71, 13.42) | −2.01 (−14.35, 10.33) | −5.67 (−15.50, 4.16) |
| P-value vs. placebo ^{a} | | 0.7535 | 0.7493 | 0.2585 |
| ≥30 | | | | |
| Number (observed/imputed) | 8 (8/0) | 9 (8/1) | 4 (4/0) | 4 (3/1) |
| Mean (SD) | −16.25 (12.62) | −15.85 (8.40) | −16.50 (13.23) | −23.25 (10.60) |
| LS Mean (SE) ^{a} | −14.22 (4.23) | −14.65 (3.89) | −15.87 (4.21) | −21.80 (5.17) |
| LS Mean Diff vs. placebo (95% CI) ^{a} | | −0.43 (−11.59, 10.72) | −1.65 (−13.49, 10.19) | −7.58 (−20.66, 5.50) |
| P-value vs. placebo ^{a} | | 0.9391 | 0.7849 | 0.2561 |
| Overall p-value for interaction ^{b} | | | | 0.9352 |

Region ^{c}

| | | | | |
|---|---|---|---|---|
| Asia | | | | |
| Number (observed/imputed) | 8 (8/0) | 9 (9/0) | 8 (8/0) | 6 (6/0) |
| Mean (SD) | −7.46 (11.36) | −9.59 (9.91) | −11.25 (10.32) | −13.93 (11.07) |
| LS Mean (SE) ^{a} | −6.47 (3.67) | −9.00 (3.50) | −11.32 (3.76) | −14.25 (3.99) |
| LS Mean Diff vs. placebo (95% CI) ^{a} | | −2.53 (−12.50, 7.44) | −4.86 (−15.18, 5.46) | −7.78 (−18.32, 2.76) |
| P-value vs. placebo ^{a} | | 0.6187 | 0.3562 | 0.1479 |
| Latin America | | | | |
| Number (observed/imputed) | 11 (10/1) | 10 (7/3) | 8 (6/2) | 6 (6/0) |
| Mean (SD) | −13.32 (15.08) | −13.28 (10.78) | −10.23 (11.81) | −14.07 (12.07) |
| LS Mean (SE) ^{a} | −11.88 (3.39) | −10.74 (3.14) | −9.26 (3.71) | −11.50 (4.23) |
| LS Mean Diff vs. placebo (95% CI) ^{a} | | 1.14 (−7.86, 10.15) | 2.62 (−7.19, 12.43) | 0.38 (−10.12, 10.88) |
| P-value vs. placebo ^{a} | | 0.8035 | 0.6003 | 0.9434 |
| Eastern Europe | | | | |
| Number (observed/imputed) | 2 (2/0) | 4 (4/0) | 3 (3/0) | 4 (4/0) |
| Mean (SD) | −10.00 (18.38) | −5.79 (5.45) | −20.14 (18.13) | −21.88 (11.40) |
| LS Mean (SE) ^{a} | −19.72 (8.80) | −5.35 (5.65) | −6.34 (10.64) | −25.97 (6.17) |
| LS Mean Diff vs. placebo (95% CI) ^{a} | | 14.37 (−6.40, 35.15) | 13.37 (−19.79, 46.54) | −6.26 (−24.55, 12.03) |
| P-value vs. placebo ^{a} | | 0.1751 | 0.4292 | 0.5025 |
| Western Countries | | | | |
| Number (observed/imputed) | 11 (11/0) | 7 (7/0) | 7 (7/0) | 12 (11/1) |
| Mean (SD) | −7.68 (12.86) | −10.70 (8.61) | −20.62 (10.65) | −23.13 (10.61) |
| LS Mean (SE) ^{a} | −7.98 (3.38) | −8.74 (4.03) | −20.19 (3.23) | −19.49 (3.03) |
| LS Mean Diff vs. placebo (95% CI) ^{a} | | −0.76 (−11.10, 9.58) | −12.21 (−21.37, −3.06) | −11.51 (−20.42, −2.61) |
| P-value vs. placebo ^{a} | | 0.8853 | 0.0090 | 0.0113 |
| Overall p-value for interaction ^{b} | | | | 0.4154 |

Race

| | | | | |
|---|---|---|---|---|
| White | | | | |
| Number (observed/imputed) | 24 (23/1) | 21 (18/3) | 18 (16/2) | 20 (19/1) |
| Mean (SD) | −10.46 (13.90) | −10.99 (9.33) | −15.92 (12.79) | −20.70 (11.84) |
| LS Mean (SE) ^{a} | −10.65 (2.36) | −9.37 (2.37) | −15.38 (2.37) | −17.97 (2.44) |
| LS Mean Diff vs. placebo (95% CI) ^{a} | | 1.28 (−5.33, 7.88) | −4.73 (−11.30, 1.84) | −7.33 (−13.95, −0.70) |
| P-value vs. placebo ^{a} | | 0.7052 | 0.1579 | 0.0303 |
| All the Others | | | | |
| Number (observed/imputed) | 8 (8/0) | 9 (9/0) | 8 (8/0) | 8 (8/0) |
| Mean (SD) | −7.46 (11.36) | −9.59 (9.91) | −11.25 (10.32) | −14.86 (9.62) |
| LS Mean (SE) ^{a} | −6.51 (3.58) | −8.43 (3.32) | −11.33 (3.66) | −14.94 (3.44) |
| LS Mean Diff vs. placebo (95% CI) ^{a} | | −1.92 (−11.55, 7.72) | −4.82 (−14.87, 5.24) | 8.43 (−18.08, 1.23) |
| P-value vs. placebo ^{a} | | 0.6967 | 0.3477 | 0.0871 |
| Overall p-value for interaction ^{b} | | | | 0.9692 |

Ethnicity

| | | | | |
|---|---|---|---|---|
| Hispanic | | | | |
| Number (observed/imputed) | 10 (10/0) | 12 (10/2) | 8 (6/2) | 8 (8/0) |
| Mean (SD) | −14.65 (15.20) | −10.26 (9.92) | −10.23 (11.81) | −15.49 (12.69) |
| LS Mean (SE) ^{a} | −12.54 (3.60) | −8.80 (2.97) | −10.31 (3.59) | −13.76 (3.78) |
| LS Mean Diff vs. placebo (95% CI) ^{a} | | 3.74 (−5.35, 12.83) | 2.24 (−7.72, 12.19) | −1.22 (−11.34, 8.91) |
| P-value vs. placebo ^{a} | | 0.4198 | 0.6597 | 0.8139 |
| Non-Hispanic | | | | |
| Number (observed/imputed) | 22 (21/1) | 18 (17/1) | 17 (17/0) | 18 (17/1) |
| Mean (SD) | −7.46 (11.88) | −10.78 (9.25) | −16.52 (12.39) | −22.02 (10.28) |
| LS Mean (SE) ^{a} | −8.25 (2.37) | 9.79 (2.59) | −16.16 (2.46) | −19.24 (2.48) |
| LS Mean Diff vs. placebo (95% CI) ^{a} | | −1.54 (−8.49, 5.41) | −7.92 (−14.63, −1.20) | −10.99 (−17.71, −4.28) |
| P-value vs. placebo ^{a} | | 0.6636 | 0.0209 | 0.0013 |
| Overall p-value for interaction ^{b} | | | | 0.2878 |

History of angioedema

| | | | | |
|---|---|---|---|---|
| Yes | | | | |
| Number (observed/imputed) | 15 (14/1) | 11 (8/3) | 8 (6/2) | 9 (8/1) |
| Mean (SD) | −10.17 (12.72) | −13.08 (11.63) | −8.60 (9.01) | −16.58 (10.23) |
| LS Mean (SE) ^{a} | −9.67 (2.91) | −11.20 (2.97) | −11.58 (3.69) | −15.33 (3.68) |
| LS Mean Diff vs. placebo (95% CI) ^{a} | | −1.53 (−9.72, 6.65) | −1.91 (−11.17, 7.35) | −5.66 (−14.82, 3.50) |
| P-value vs. placebo ^{a} | | 0.7135 | 0.6863 | 0.2255 |
| No | | | | |
| Number (observed/imputed) | 17 (17/0) | 19 (19/0) | 18 (18/0) | 19 (19/0) |
| Mean (SD) | −9.30 (13.98) | −9.12 (7.74) | −17.10 (12.56) | −20.20 (12.00) |
| LS Mean (SE) ^{a} | −9.88 (2.69) | −7.72 (2.58) | −15.01 (2.39) | −17.97 (2.40) |
| LS Mean Diff vs. placebo (95% CI) ^{a} | | 2.16 (−5.19, 9.52) | −5.13 (−12.18, 1.92) | −8.09 (−15.14, −1.03) |
| P-value vs. placebo ^{a} | | 0.5644 | 0.1538 | 0.0246 |
| Overall p-value for interaction ^{b} | | | | 0.7199 |

Baseline UAS7 score

| | | | | |
|---|---|---|---|---|
| <28 | | | | |
| Number (observed/imputed) | 12 (12/0) | 6 (6/0) | 11 (9/2) | 11 (11/0) |
| Mean (SD) | −3.75 (12.65) | −8.39 (3.86) | −11.43 (8.06) | −13.43 (7.05) |
| LS Mean (SE) ^{a} | −4.40 (2.72) | −5.87 (3.45) | −8.89 (2.76) | −11.75 (2.89) |
| LS Mean Diff vs. placebo (95% CI) ^{a} | | −1.46 (−10.06, 7.14) | −4.49 (−12.35, 3.38) | −7.34 (−15.21, 0.52) |
| P-value vs. placebo ^{a} | | 0.7388 | 0.2634 | 0.0670 |
| ≥28 | | | | |
| Number (observed/imputed) | 20 (19/1) | 24 (21/3) | 15 (15/0) | 17 (16/1) |
| Mean (SD) | −13.28 (12.47) | −11.12 (10.28) | −16.73 (14.20) | −22.66 (12.35) |
| LS Mean (SE) ^{a} | −13.11 (2.72) | −11.17 (2.39) | −17.23 (2.81) | −19.91 (2.64) |
| LS Mean Diff vs. placebo (95% CI) ^{a} | | 1.94 (−5.16, 9.03) | −4.13 (−11.78, 3.52) | −6.81 (−14.23, 0.62) |
| P-value vs. placebo ^{a} | | 0.5925 | 0.2903 | 0.0722 |
| Overall p-value for interaction ^{b} | | | | 0.9282 |

Baseline ISS7 score

| | | | | |
|---|---|---|---|---|
| <13 | | | | |
| Number (observed/imputed) | 10 (10/0) | 2 (2/0) | 6 (5/1) | 2 (2/0) |
| Mean (SD) | −5.50 (12.18) | −5.83 (3.06) | −10.78 (8.33) | −10.75 (6.01) |
| LS Mean (SE) ^{a} | −4.91 (3.05) | −3.27 (5.21) | −9.30 (4.02) | 9.24 (6.48) |
| LS Mean Diff vs. placebo (95% CI) ^{a} | | 1.64 (−10.30, 13.58) | −4.39 (−14.52, 5.74) | −4.33 (−18.30, 9.63) |
| P-value vs. placebo ^{a} | | 0.7870 | 0.3954 | 0.5433 |
| ≥13 | | | | |
| Number (observed/imputed) | 22 (21/1) | 28 (25/3) | 20 (19/1) | 26 (25/1) |
| Mean (SD) | −11.62 (13.46) | −10.91 (9.59) | −15.60 (12.97) | −19.67 (11.53) |
| LS Mean (SE) ^{a} | −10.75 (2.45) | −10.47 (2.13) | −15.07 (2.33) | −18.70 (2.15) |
| LS Mean Diff vs. placebo (95% CI) ^{a} | | 0.28 (−6.08, 6.64) | −4.32 (−10.91, 2.27) | −7.95 (−14.32, −1.57) |
| P-value vs. placebo ^{a} | | 0.9304 | 0.1991 | 0.0145 |
| Overall p-value for interaction ^{b} | | | | 0.8561 |

Duration of disease (years)

| | | | | |
|---|---|---|---|---|
| <2 | | | | |
| Number (observed/imputed) | 14 (13/1) | 10 (9/1) | 14 (13/1) | 12 (11/1) |
| Mean (SD) | −7.17 (10.41) | −12.50 (9.27) | −14.54 (10.00) | −19.20 (11.00) |
| LS Mean (SE) ^{a} | −7.07 (2.94) | −10.62 (3.36) | −16.30 (2.66) | −17.20 (3.03) |
| LS Mean Diff vs. placebo (95% CI) ^{a} | | −3.55 (−12.28, 5.17) | −9.24 (−17.02, −1.46) | −10.14 (−18.42, −1.86) |
| P-value vs. placebo ^{a} | | 0.4248 | 0.0200 | 0.0164 |
| 2-10 | | | | |
| Number (observed/imputed) | 15 (15/0) | 16 (16/0) | 11 (10/1) | 12 (12/0) |
| Mean (SD) | −13.42 (14.67) | −10.83 (9.70) | −15.56 (14.75) | −17.51 (11.39) |
| LS Mean (SE) ^{a} | −12.83 (2.86) | −9.73 (2.58) | −12.88 (3.03) | −16.44 (3.07) |
| LS Mean Diff vs. placebo (95% CI) ^{a} | | 3.11 (−4.51, 10.72) | −0.05 (−8.24, 8.14) | −3.61 (−11.80, 4.58) |
| P-value vs. placebo ^{a} | | 0.4239 | 0.9909 | 0.3878 |
| >10 | | | | |
| Number (observed/imputed) | 3 (3/0) | 4 (2/2) | 1 (1/0) | 4 (4/0) |
| Mean (SD) | −3.00 (16.52) | −4.70 (7.74) | −1.83 (NC) | −23.13 (14.76) |
| LS Mean (SE) ^{a} | −5.92 (6.96) | −1.05 (6.12) | 8.87 (12.67) | −18.73 (5.22) |
| LS Mean Diff vs. placebo (95% CI) ^{a} | | 4.87 (−13.68, 23.41) | 14.79 (−14.26, 43.85) | −12.81 (−29.81, 4.20) |
| P-value vs. placebo ^{a} | | 0.6071 | 0.3183 | 0.1399 |
| Overall p-value for interaction ^{b} | | | | 0.5067 |

Baseline H1-antihistamines dose

| | | | | |
|---|---|---|---|---|
| 1-fold | | | | |
| Number (observed/imputed) | 17 (17/0) | 16 (14/2) | 11 (10/1) | 18 (17/1) |
| Mean (SD) | −12.60 (13.61) | −8.61 (9.50) | −18.22 (14.88) | −15.87 (11.07) |
| LS Mean (SE) ^{a} | −12.27 (2.76) | −7.64 (2.73) | −15.75 (3.11) | −14.92 (2.59) |
| LS Mean Diff vs. placebo (95% CI) ^{a} | | 4.64 (−2.96, 12.23) | −3.48 (−11.69, 4.73) | −2.65 (−10.05, 4.76) |
| P-value vs. placebo ^{a} | | 0.2316 | 0.4057 | 0.4837 |
| 2 to 4-fold standard dose | | | | |
| Number (observed/imputed) | 14 (13/1) | 14 (13/1) | 15 (14/1) | 10 (10/0) |
| Mean (SD) | −6.32 (12.80) | −12.81 (9.00) | −11.75 (9.14) | −24.73 (10.10) |
| LS Mean (SE) ^{a} | −6.56 (2.90) | −10.93 (2.77) | −12.98 (2.55) | −20.96 (3.15) |
| LS Mean Diff vs. placebo (95% CI) ^{a} | | −4.37 (−12.30, 3.56) | −6.42 (−13.94, 1.10) | −14.41 (−22.75, −6.07) |
| P-value vs. placebo ^{a} | | 0.2798 | 0.0942 | 0.0007 |
| Overall p-value for interaction ^{b} | | | | 0.1610 |

Baseline Eosinophils (10\hat{ }9/L)

| | | | | |
|---|---|---|---|---|
| <0.150 | | | | |
| Number | 16 | N/A | 11 | 14 |
| Mean (SD) | N/A | N/A | N/A | N/A |
| LS Mean (SE) | −13.06 (2.87) | N/A | −13.60 (3.19) | −20.09 (3.11) |
| LS Mean Diff vs. placebo (95% CI) | | N/A | −0.54 (−8.95, 7.87) | −7.04 (−15.34, 1.27) |
| P-value vs. placebo | | N/A | N/A | N/A |
| ≥0.150 | | | | |
| Number | 16 | N/A | 15 | 14 |
| Mean (SD) | N/A | N/A | N/A | N/A |
| LS Mean (SE) | −6.89 (2.77) | N/A | −14.57 (2.57) | −14.80 (2.58) |
| LS Mean Diff vs. placebo (95% CI) | | N/A | −7.68 (−15.25, −0.11) | −7.92 (−15.27, −0.56) |
| P-value vs. placebo | | N/A | N/A | N/A |

Baseline Eosinophils (10\hat{ }9/L)

| | | | | |
|---|---|---|---|---|
| <0.165 (Median) | | | | |
| Number (observed/imputed) | 17 (17/0) | 16 (14/2) | 13 (12/1) | 14 (14/0) |
| Mean (SD) | −12.63 (13.99) | 9.46 (8.63) | −11.24 (8.98) | −21.57 (13.01) |
| LS Mean (SE) ^{a} | −12.47 (2.71) | −7.71 (2.71) | −14.30 (2.83) | −19.82 (2.96) |
| LS Mean Diff vs. placebo (95% CI) ^{a} | | 4.76 (−2.75, 12.26) | −1.83 (−9.50, 5.84) | −7.36 (−15.23, 0.52) |
| P-value vs. placebo ^{a} | | 0.2139 | 0.6397 | 0.0673 |
| ≥0.165 (Median) | | | | |
| Number (observed/imputed) | 15 (14/1) | 14 (13/1) | 13 (12/1) | 14 (13/1) |
| Mean (SD) | −6.40 (11.83) | −11.84 (10.30) | −17.73 (14.16) | −16.50 (9.30) |
| LS Mean (SE) ^{a} | −7.12 (2.93) | −10.83 (2.83) | −14.13 (2.87) | −14.63 (2.71) |
| LS Mean Diff vs. placebo (95% CI) ^{a} | | −3.71 (−11.80, 4.38) | −7.01 (−15.24, 1.22) | −7.52 (−15.32, 0.28) |
| P-value vs. placebo ^{a} | | 0.3686 | 0.0948 | 0.0590 |
| Overall p-value for interaction ^{b} | | | | 0.3253 |

Baseline Eosinophils (10\hat{ }9/L)

| | | | | |
|---|---|---|---|---|
| <0.3 | | | | |
| Number (observed/imputed) | 29 (28/1) | 25 (22/3) | 20 (19/1) | 23 (22/1) |
| Mean (SD) | −9.51 (13.70) | −10.75 (8.91) | −14.90 (12.30) | −19.99 (12.00) |
| LS Mean (SE) ^{a} | −10.16 (2.13) | −9.19 (2.15) | −15.05 (2.31) | −18.29 (2.31) |
| LS Mean Diff vs. placebo (95% CI) ^{a} | | 0.97 (−5.00, 6.93) | −4.89 (−11.04, 1.26) | −8.14 (−14.29, −1.98) |
| P-value vs. placebo ^{a} | | 0.7505 | 0.1188 | 0.0096 |
| ≥0.3 | | | | |
| Number (observed/imputed) | 3 (3/0) | 5 (5/0) | 6 (5/1) | 5 (5/0) |
| Mean (SD) | −11.67 (8.14) | −9.69 (12.55) | −13.11 (12.32) | −14.65 (7.48) |
| LS Mean (SE) ^{a} | −7.66 (5.36) | −9.66 (4.84) | −11.06 (3.93) | −13.16 (4.09) |
| LS Mean Diff vs. placebo (95% CI) ^{a} | | −2.00 (−16.22, 12.21) | −3.40 (−16.66, 9.86) | −5.51 (−18.58, 7.57) |
| P-value vs. placebo ^{a} | | 0.7820 | 0.6148 | 0.4085 |
| Overall p-value for interaction ^{b} | | | | 0.9380 |

Baseline Total IgE (IU/mL)

| | | | | |
|---|---|---|---|---|
| <300 | | | | |
| Number (observed/imputed) | 23 (22/1) | 21 (18/3) | 18 (16/2) | 18 (18/0) |
| Mean (SD) | −7.80 (13.81) | −10.61 (9.62) | 15.35 (12.23) | −20.66 (12.39) |
| LS Mean (SE) ^{a} | −8.06 (2.36) | −9.07 (2.38) | −14.78 (2.46) | −18.19 (2.47) |
| LS Mean Diff vs. placebo (95% CI) ^{a} | | −1.01 (−7.60, 5.59) | −6.72 (−13.41, −0.03) | −10.13 (−16.83, −3.44) |
| P-value vs. placebo ^{a} | | 0.7649 | 0.0490 | 0.0030 |
| ≥300 | | | | |
| Number (observed/imputed) | 9 (9/0) | 8 (8/0) | 8 (8/0) | 10 (9/1) |
| Mean (SD) | −14.59 (10.63) | −9.60 (9.52) | −12.53 (12.31) | −16.12 (9.22) |
| LS Mean (SE) ^{a} | −12.46 (3.64) | −8.04 (3.57) | −14.22 (3.59) | −14.94 (3.46) |
| LS Mean Diff vs. placebo (95% CI) ^{a} | | 4.42 (−5.58, 14.42) | −1.77 (−12.00, 8.47) | −2.48 (−12.37, 7.41) |
| P-value vs. placebo ^{a} | | 0.3860 | 0.7351 | 0.6230 |
| Overall p-value for interaction ^{b} | | | | 0.5123 |

Baseline Total IgE (IU/mL)

| | | | | |
|---|---|---|---|---|
| <100 | | | | |
| Number (observed/imputed) | 15 (14/1) | 11 (10/1) | 11 (10/1) | 13 (13/0) |
| Mean (SD) | −6.59 (15.16) | −11.05 (9.70) | −16.26 (11.38) | −21.03 (13.37) |
| LS Mean (SE) ^{a} | −7.30 (3.09) | 8.49 (3.25) | −15.72 (3.24) | −17.40 (3.01) |
| LS Mean Diff vs. placebo (95% CI) ^{a} | | −1.19 (−10.05, 7.67) | −8.43 (−17.18, 0.33) | −10.10 (−18.54, −1.65) |
| P-value vs. placebo ^{a} | | 0.7926 | 0.0593 | 0.0191 |
| ≥100 | | | | |
| Number (observed/imputed) | 17 (17/0) | 18 (16/2) | 15 (14/1) | 15 (14/1) |
| Mean (SD) | −12.46 (10.92) | −9.90 (9.51) | −13.19 (12.80) | −17.31 (9.51) |
| LS Mean (SE) ^{a} | −11.51 (2.59) | −8.97 (2.49) | −13.17 (2.55) | −17.08 (2.69) |
| LS Mean Diff vs. placebo (95% CI) ^{a} | | 2.53 (−4.52, 9.59) | −1.67 (−8.84, 5.51) | −5.57 (−12.83, 1.69) |
| P-value vs. placebo ^{a} | | 0.4814 | 0.6486 | 0.1328 |
| Overall p-value for interaction ^{b} | | | | 0.6477 |

Baseline BHRA

| | | | | |
|---|---|---|---|---|
| BHRA+ | | | | |
| Number (observed/imputed) | 3 (3/0) | 3 (2/1) | 6 (5/1) | 4 (4/0) |
| Mean (SD) | −8.83 (8.78) | −19.94 (5.28) | −19.50 (10.03) | −22.13 (16.40) |
| LS Mean (SE) ^{a} | −9.49 (6.28) | −17.03 (6.45) | −19.58 (5.46) | −22.12 (5.72) |
| LS Mean Diff vs. placebo (95% CI) ^{a} | | −7.53 (−24.53, 9.46) | −10.09 (−27.02, 6.84) | −12.63 (−29.25, 4.00) |
| P-value vs. placebo ^{a} | | 0.3849 | 0.2428 | 0.1365 |
| BHRA− | | | | |
| Number (observed/imputed) | 29 (28/1) | 26 (24/2) | 20 (19/1) | 24 (23/1) |
| Mean (SD) | −9.80 (13.68) | −9.96 (9.08) | −12.98 (12.46) | −18.52 (10.75) |
| LS Mean (SE) ^{a} | −9.97 (2.05) | −8.95 (2.08) | −12.39 (2.21) | −16.51 (2.11) |
| LS Mean Diff vs. placebo (95% CI) ^{a} | | 1.02 (−4.73, 6.78) | −2.42 (−8.35, 3.52) | −6.53 (−12.28, −0.79) |
| P-value vs. placebo ^{a} | | 0.7271 | 0.4247 | 0.0258 |
| Overall p-value for interaction ^{b} | | | | 0.4945 |

^{a} Each of the imputed complete data were analyzed by fitting an ANCOVA model with the corresponding baseline value and intervention group as covariates. Analysis was based on the same imputed dataset using WOCF/MI from primary analysis of the primary endpoint.
^{b} Each of the imputed complete data were analyzed by fitting an ANCOVA model with the corresponding baseline value and intervention group as covariates, plus the subgroup variable and the subgroup-by-intervention interaction. Analysis was based on the same imputed dataset using WOCF/MI from primary analysis of the primary endpoint.
^{c} Asia: South Korea, Taiwan, Japan; Latin America: Argentina, Chile; Eastern Europe: Poland; Western Countries: Spain, Italy, Germany, Canada, Greece, Netherlands.
Note:
Descriptive statistics include participants with WOCF imputation at Week 12 and participants whose Week 12 values were imputed by MI were excluded from the descriptive analysis.

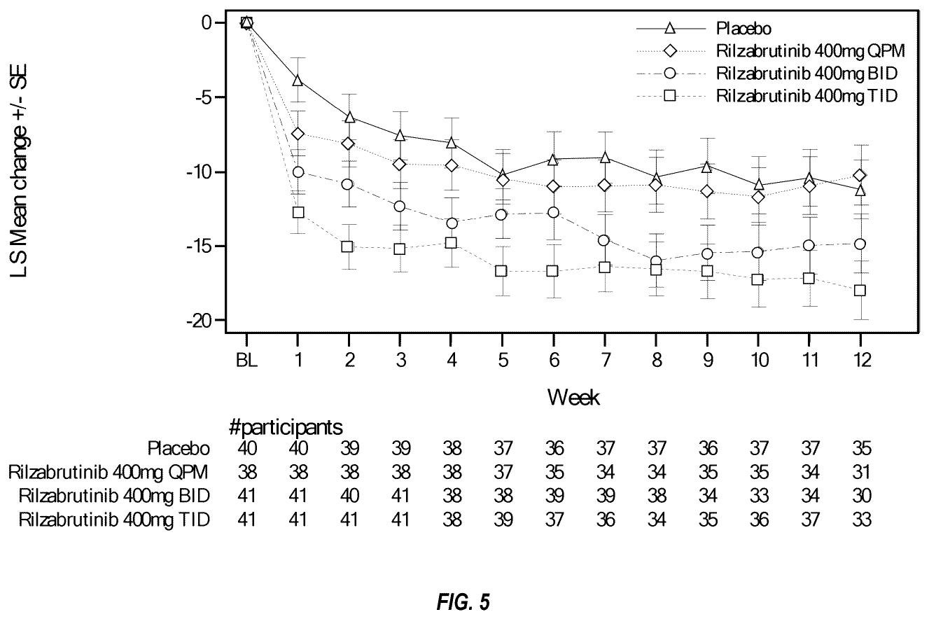

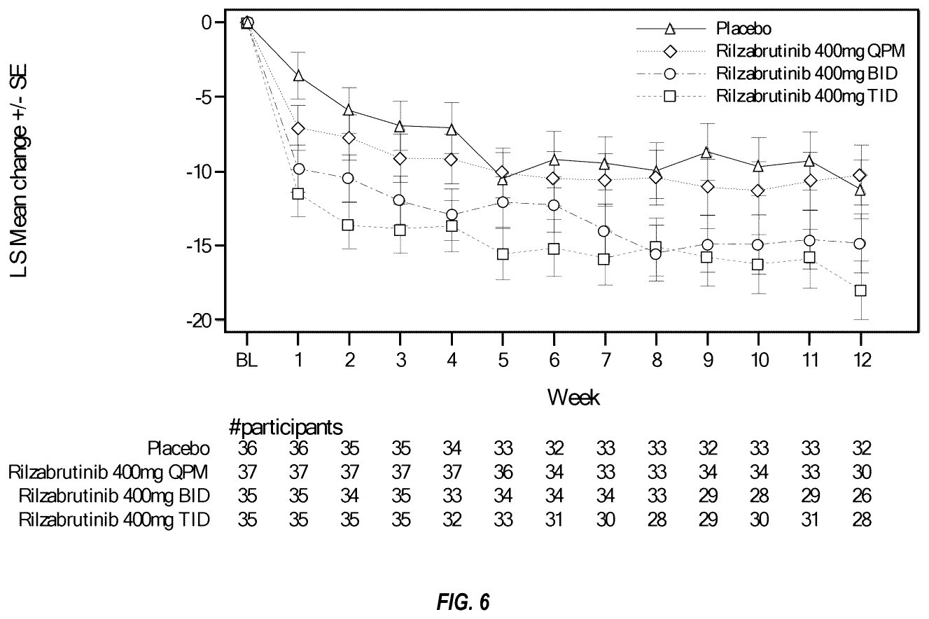

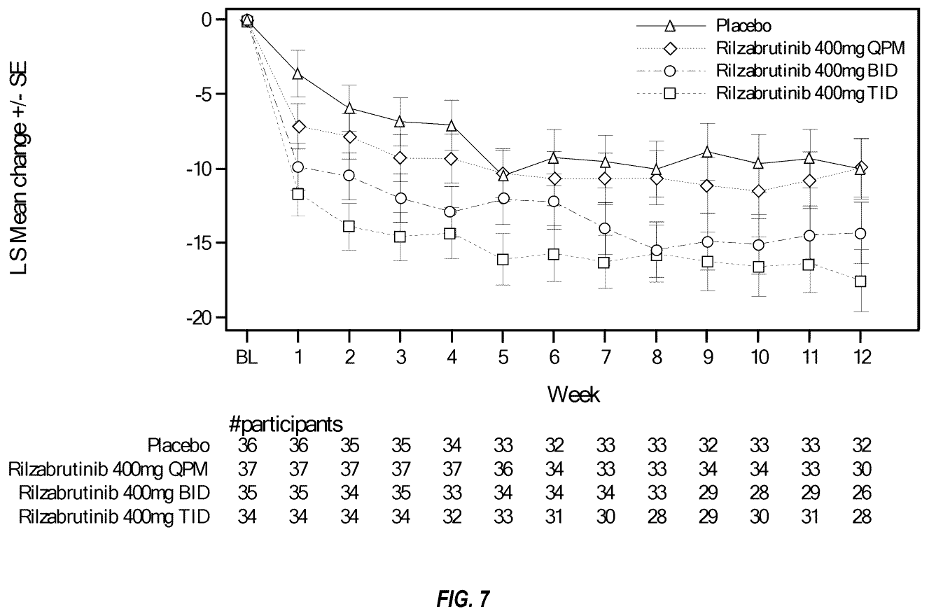

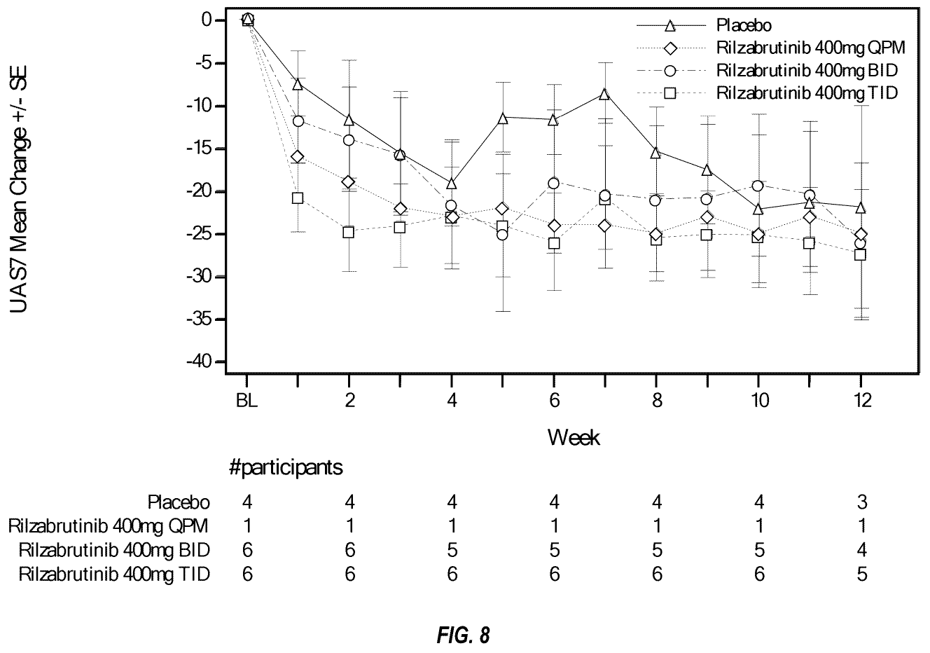

Additionally, participants exhibited a rapid and sustained improvement in symptoms, with significant reductions in UAS7 observed following one week of treatment (see FIGS. 5-8 and Tables 17-20). In the ITT population, participants receiving 400 mg BID rilzabrutinib had an LS mean change in UAS7 from baseline to Week 12 of −10.01 (p=0.0024; see Table 18). Consistent with rilzabrutinib's dose-dependent effect on UAS7, participants receiving 400 mg TID showed an LS mean change in UAS7 from baseline to Week 12 of −12.72 (p<0.0001), reflecting a 40.18% decrease from baseline. Notably, the overall reduction in UAS7 observed in both the 400 mg BID and TID arms exceeded the minimal clinically important difference (MCID) of 10 points.

Similar results were observed for the omalizumab-naïve population (see Table 19). Omalizumab-naïve participants in the 400 mg BID and TID rilzabrutinib arms experienced an LS mean reduction in UAS7 of −9.79 (p=0.0040) and −11.44 (p=0.0003) from baseline to Week 12, respectively. Similar data were obtained when these analyses were repeated following the removal of the asymptomatic outlier (see Table 20).

TABLE 17
Summary of UAS7 by Week in the Double−Blind Period (Omalizumab-Naïve Population and Patients with Prior Omalizumab Use, Including Omalizumab-Incomplete Responders)

| | Omalizumab-Naïve Population | | | | Patients with Prior Omalizumab Use, Including Omalizumab-Incomplete Responders | | | |
|---|---|---|---|---|---|---|---|---|
| | | Rilzabrutinib | Rilzabrutinib | Rilzabrutinib | | Rilzabrutinib | Rilzabrutinib | Rilzabrutinib |
| | Placebo | 400 mg QPM | 400 mg BID | 400 mg TID | Placebo | 400 mg QPM | 400 mg BID | 400 mg TID |
| UAS7 | (N = 36) | (N = 37) | (N = 35) | (N = 35) | (N = 4) | (N = 1) | (N = 6) | (N = 6) |
| Baseline | | | | | | | | |
| Number | 36 | 37 | 35 | 35 | 4 | 1 | 6 | 6 |
| Mean (SD) | 28.97 (8.39) | 31.11 (7.24) | 29.73 (7.11) | 29.34 (8.08) | 38.96 (3.53) | 41.00 (NC) | 33.00 (7.67) | 33.03 (11.40) |
| Median | 28.00 | 30.00 | 28.00 | 28.00 | 39.92 | 41.00 | 35.00 | 38.00 |
| Q1; Q3 | 22.00; 35.00 | 27.00; 38.00 | 24.50; 35.00 | 25.00; 35.00 | 36.50; 41.42 | 41.00; 41.00 | 27.00; 38.00 | 22.17; 42.00 |
| Min; Max | 16.0; 42.0 | 16.0; 42.0 | 18.0; 42.0 | 0.0; 42.0 | 34.0; 42.0 | 41.0; 41.0 | 21.0; 42.0 | 16.0; 42.0 |
| Week 1 | | | | | | | | |
| Number (observed/imputed) | 36 (36/0) | 37 (37/0) | 35 (35/0) | 35 (35/0) | 4 (4/0) | 1 (1/0) | 6 (5/1) | 6 (6/0) |
| Mean (SD) | 25.80 (10.44) | 23.81 (8.78) | 19.77 (11.32) | 17.77 (10.26) | 31.50 (9.04) | 25.00 (NC) | 21.17 (12.48) | 12.17 (8.54) |
| Median | 27.00 | 25.00 | 21.00 | 18.00 | 31.50 | 25.00 | 23.50 | 12.00 |
| Q1; Q3 | 21.50; 32.50 | 18.00; 29.00 | 10.00; 28.00 | 8.00; 27.00 | 24.50; 38.50 | 25.00; 25.00 | 14.00; 30.00 | 4.00; 16.00 |
| Min; Max | 0.0; 42.0 | 5.0; 42.0 | 0.0; 42.0 | 0.0; 35.0 | 21.0; 42.0 | 25.0; 25.0 | 1.0; 35.0 | 3.0; 26.0 |
| Change from baseline | | | | | | | | |
| Number (observed/imputed) | 36 (36/0) | 37 (37/0) | 35 (35/0) | 35 (35/0) | 4 (4/0) | 1 (1/0) | 6 (5/1) | 6 (6/0) |
| Mean (SD) | −3.17 (8.91) | −7.30 (9.14) | −9.96 (8.81) | −11.57 (10.61) | −7.46 (7.56) | −16.00 (NC) | −11.83 (12.14) | −20.86 (9.80) |
| Median | 0.50 | −6.00 | −9.00 | −10.00 | −5.92 | −16.00 | −7.50 | −21.00 |
| Q1; Q3 | −7.50; 1.50 | 12.00; −1.00 | −19.00; −4.00 | −21.00; −3.00 | −12.00; −2.92 | −16.00; −16.00 | −26.00; −2.00 | −30.00; −13.00 |
| Min; Max | −30.0; 17.0 | −35.0; 8.0 | −29.0; 3.0 | −36.0; 5.0 | −18.0; 0.0 | −16.0; −16.0 | −28.0; 0.0 | −32.0; −8.2 |
| Percent change from baseline | | | | | | | | |
| Number (observed/imputed) | 36 (36/0) | 37 (37/0) | 35 (35/0) | 34 (34/0) | 4 (4/0) | 1 (1/0) | 6 (5/1) | 6 (6/0) |
| Mean (SD) | −9.17 (34.38) | −21.72 (26.65) | −35.41 (33.69) | −38.05 (33.56) | −19.52 (19.33) | −39.02 (NC) | −35.59 (37.55) | −63.75 (22.12) |
| Median | −1.56 | −17.95 | −26.19 | −30.95 | −15.97 | −39.02 | −20.53 | −69.05 |
| Q1; Q3 | −31.18; 4.70 | −36.36; −3.33 | −61.54; −13.79 | −65.63; −13.79 | −31.90; −7.14 | −39.02; −39.02 | −66.67; −9.52 | −81.25; −38.10 |
| Min; Max | −100.0; 70.8 | −87.5; 27.6 | −100.0; 12.0 | −94.4; 17.9 | 46.2; 0.0 | −39.0; −39.0 | −96.3; 0.0 | −88.2; −36.8 |
| Week 2 | | | | | | | | |
| Number (observed/imputed) | 35 (35/0) | 37 (37/0) | 34 (34/0) | 35 (35/0) | 4 (4/0) | 1 (1/0) | 6 (5/1) | 6 (6/0) |
| Mean (SD) | 23.49 (10.05) | 23.44 (8.97) | 19.59 (10.86) | 15.65 (10.66) | 27.29 (15.37) | 22.00 (NC) | 19.00 (13.67) | 8.33 (9.77) |
| Median | 25.00 | 23.00 | 18.10 | 14.00 | 29.58 | 22.00 | 23.00 | 5.50 |
| Q1; Q3 | 18.00; 29.00 | 20.00; 29.00 | 11.00; 28.00 | 7.00; 22.40 | 15.08; 39.50 | 22.00; 22.00 | 5.00; 28.00 | 0.00; 14.00 |
| Min; Max | 0.0; 42.0 | 6.0; 42.0 | 0.0; 42.0 | 0.0; 38.0 | 8.0; 42.0 | 22.0; 22.0 | 0.0; 35.0 | 0.0; 25.0 |
| Change from baseline | | | | | | | | |
| Number (observed/imputed) | 35 (35/0) | 37 (37/0) | 34 (34/0) | 35 (35/0) | 4 (4/0) | 1 (1/0) | 6 (5/1) | 6 (6/0) |
| Mean (SD) | −5.11 (9.01) | −7.67 (8.84) | −10.10 (8.61) | −13.69 (11.66) | −11.67 (13.80) | −19.00 (NC) | −14.00 (14.98) | −24.69 (11.86) |
| Median | −3.00 | −5.00 | −10.00 | −11.00 | −7.83 | −19.00 | −10.00 | −22.50 |
| Q1; Q3 | −8.00; 0.00 | −13.00; 0.00 | −14.00; −2.00 | −23.00; −5.60 | −21.42; −1.92 | −19.00; −19.00 | −27.00; 0.00 | −34.00; −14.17 |
| Min; Max | −32.0; 12.0 | −32.0; 2.5 | −31.0; 3.0 | −35.0; 7.0 | −31.0; 0.0 | −19.0; −19.0 | −37.0; 0.0 | −42.0; −13.0 |
| Percent change from baseline | | | | | | | | |
| Number (observed/imputed) | 35 (35/0) | 37 (37/0) | 34 (34/0) | 34 (34/0) | 4 (4/0) | 1 (1/0) | 6 (5/1) | 6 (6/0) |
| Mean (SD) | −16.27 (32.44) | −23.23 (26.34) | −35.50 (31.03) | −45.70 (36.18) | −30.92 (35.56) | −46.34 (NC) | −40.50 (43.43) | −75.38 (23.12) |
| Median | −9.38 | −16.67 | −35.71 | −46.67 | −22.10 | −46.34 | −27.44 | −73.96 |
| Q1; Q3 | −30.95; 0.00 | −39.39; 0.00 | −50.00; −11.11 | −80.56; −21.08 | −57.15; −4.69 | −46.34; −46.34 | −88.10; 0.00 | −100.00; −63.91 |
| Min; Max | −100.0; 50.0 | −80.6; 9.1 | −100.0; 13.3 | −100.0; 25.0 | −79.5; 0.0 | −46.3; −46.3 | −100.0; 0.0 | −100.0; −40.5 |
| Week 3 | | | | | | | | |
| Number (observed/imputed) | 35 (35/0) | 37 (37/0) | 35 (35/0) | 35 (35/0) | 4 (4/0) | 1 (1/0) | 6 (5/1) | 6 (6/0) |
| Mean (SD) | 22.60 (10.89) | 22.01 (9.00) | 17.71 (11.91) | 15.37 (9.87) | 23.30 (14.48) | 19.00 (NC) | 17.19 (13.86) | 8.89 (7.68) |
| Median | 23.00 | 22.00 | 16.80 | 14.00 | 26.43 | 19.00 | 18.58 | 7.67 |
| Q1; Q3 | 17.50; 30.00 | 15.00; 28.00 | 10.00; 25.00 | 7.00; 21.00 | 14.63; 31.97 | 19.00; 19.00 | 3.00; 28.00 | 3.00; 14.00 |
| Min; Max | 0.0; 42.0 | 5.0; 42.0 | 0.0; 42.0 | 0.0; 35.0 | 3.0; 37.3 | 19.0; 19.0 | 0.0; 35.0 | 0.0; 21.0 |
| Change from baseline | | | | | | | | |
| Number (observed/imputed) | 35 (35/0) | 37 (37/0) | 35 (35/0) | 35 (35/0) | 4 (4/0) | 1 (1/0) | 6 (5/1) | 6 (6/0) |
| Mean (SD) | −6.00 (9.80) | −9.10 (8.78) | −12.02 (10.63) | −13.97 (12.14) | −15.66 (14.49) | −22.00 (NC) | −15.81 (16.39) | −24.14 (11.99) |
| Median | −3.00 | −7.00 | −9.00 | −14.00 | −11.58 | −22.00 | −15.00 | −24.50 |
| Q1; Q3 | −12.00; 0.00 | −15.33; −2.00 | −21.20; −2.00 | −23.00; −7.00 | −25.88; −5.45 | −22.00; −22.00 | −24.00; 0.00 | −31.00; −12.83 |
| Min; Max | −33.0; 7.0 | −27.0; 2.0 | −39.0; 1.0 | −35.0; 21.0 | −36.0; −3.5 | −22.0; −22.0 | −42.0; 1.2 | −42.0; −10.0 |
| Percent change from baseline | | | | | | | | |
| Number (observed/imputed) | 35 (35/0) | 37 (37/0) | 35 (35/0) | 34 (34/0) | 4 (4/0) | 1 (1/0) | 6 (5/1) | 6 (6/0) |
| Mean (SD) | −19.11 (37.23) | −28.09 (25.63) | −41.11 (35.05) | −48.87 (33.07) | −40.04 (36.80) | −53.66 (NC) | −44.47 (44.78) | −71.37 (19.75) |
| Median | −9.00 | −26.67 | −32.14 | −48.08 | −29.63 | −53.66 | −41.73 | −64.58 |
| Q1; Q3 | −36.67; 0.00 | −46.15; −9.09 | −69.05; −8.00 | −76.67; −25.00 | −64.90; −15.17 | −53.66; −53.66 | −88.89; 0.00 | −91.18; −57.89 |
| Min; Max | −100.0; 43.8 | −82.8; 10.0 | −100.0; 5.6 | −100.0; 28.0 | −92.3; −8.6 | −53.7; −53.7 | −100.0; 5.6 | −100.0; −50.0 |
| Week 4 | | | | | | | | |
| Number (observed/imputed) | 34 (34/0) | 37 (36/1) | 33 (33/0) | 32 (32/0) | 4 (4/0) | 1 (1/0) | 5 (4/1) | 6 (6/0) |
| Mean (SD) | 22.09 (12.20) | 21.92 (8.82) | 15.92 (11.71) | 14.65 (9.61) | 19.83 (10.96) | 18.00 (NC) | 13.60 (16.55) | 10.08 (10.62) |
| Median | 23.50 | 21.00 | 14.00 | 14.00 | 20.65 | 18.00 | 3.00 | 8.58 |
| Q1; Q3 | 14.00; 29.00 | 17.00; 28.00 | 6.00; 22.17 | 8.50; 19.00 | 11.75; 27.90 | 18.00; 18.00 | 1.00; 28.00 | 2.33; 11.00 |
| Min; Max | 0.0; 42.0 | 2.0; 42.0 | 0.0; 42.0 | 0.0; 38.0 | 6.0; 32.0 | 18.0; 18.0 | 1.0; 35.0 | 0.0; 30.0 |
| Change from baseline | | | | | | | | |
| Number (observed/imputed) | 34 (34/0) | 37 (36/1) | 33 (33/0) | 32 (32/0) | 4 (4/0) | 1 (1/0) | 5 (4/1) | 6 (6/0) |
| Mean (SD) | −6.64 (11.17) | −9.18 (9.24) | −13.49 (10.97) | −15.51 (9.79) | −19.13 (10.10) | −23.00 (NC) | −21.80 (16.62) | −22.94 (13.84) |
| Median | −3.42 | −7.00 | −11.00 | −14.00 | −17.35 | −23.00 | −26.00 | −22.50 |
| Q1; Q3 | −14.00; 0.00 | −14.00; −1.00 | −22.50; −5.00 | −22.50; −8.50 | −25.60; −12.67 | −23.00; −23.00 | −32.00; −10.00 | −31.67; −12.00 |
| Min; Max | −35.0; 12.0 | −30.3; 1.0 | −38.0; 3.0 | −35.0; 2.0 | −33.0; −8.8 | −23.0; −23.0 | −41.0; 0.0 | −42.0; −7.0 |
| Percent change from baseline | | | | | | | | |
| Number (observed/imputed) | 34 (34/0) | 37 (36/1) | 33 (33/0) | 32 (32/0) | 4 (4/0) | 1 (1/0) | 5 (4/1) | 6 (6/0) |
| Mean (SD) | −21.54 (41.63) | −27.91 (26.53) | −46.55 (35.69) | −51.19 (29.70) | −49.53 (26.13) | −56.10 (NC) | −62.33 (45.90) | −67.07 (27.75) |
| Median | −13.83 | −21.88 | −41.94 | −51.09 | −45.93 | −56.10 | −91.43 | −68.48 |
| Q1; Q3 | −55.56; 0.00 | −48.39; −4.55 | −76.92; −18.52 | −73.39; −30.70 | −66.57; −32.48 | −56.10; −56.10 | −96.30; −26.32 | −93.14; −43.75 |
| Min; Max | −100.0; 66.7 | −93.1; 3.4 | −100.0; 10.3 | −100.0; 5.6 | −84.6; −21.6 | −56.1 ;−56.1 | −97.6; 0.0 | −100.0; −28.6 |
| Week 5 | | | | | | | | |
| Number (observed/imputed) | 33 (33/0) | 36 (35/1) | 34 (33/1) | 33 (33/0) | 4 (4/0) | 1 (1/0) | 4 (3/1) | 6 (5/1) |
| Mean (SD) | 18.31 (9.22) | 20.63 (9.18) | 17.22 (13.36) | 13.41 (10.60) | 27.52 (11.12) | 19.00 (NC) | 9.75 (16.94) | 8.92 (10.94) |
| Median | 16.33 | 18.63 | 16.50 | 14.00 | 28.38 | 19.00 | 2.00 | 5.50 |
| Q1; Q3 | 12.00; 24.50 | 15.28; 27.50 | 5.00; 24.00 | 5.00; 19.83 | 18.00; 37.04 | 19.00; 19.00 | 0.00; 19.50 | 2.00; 10.50 |
| Min; Max | 0.0; 42.0 | 0.0; 42.0 | 0.0; 42.0 | 0.0; 38.0 | 16.0; 37.3 | 19.0; 19.0 | 0.0; 35.0 | 0.0; 30.0 |
| Change from baseline | | | | | | | | |
| Number (observed/imputed) | 33 (33/0) | 36 (35/1) | 34 (33/1) | 33 (33/0) | 4 (4/0) | 1 (1/0) | 4 (3/1) | 6 (5/1) |
| Mean (SD) | −10.66 (8.95) | −10.65 (9.52) | −12.56 (11.55) | −16.74 (11.67) | −11.44 (8.17) | −22.00 (NC) | −25.00 (18.42) | −24.11 (14.81) |
| Median | −10.00 | −8.50 | −12.67 | −15.17 | −11.33 | −22.00 | −29.00 | −20.50 |
| Q1; Q3 | −17.00; −4.00 | −15.33; −2.88 | −21.67; −2.00 | −24.00; −9.00 | −18.50; −4.38 | −22.00; −22.00 | −38.50; −11.50 | −40.00; −11.67 |
| Min; Max | −33.0; 7.0 | −31.0; 1.0 | −33.8; 13.0 | −42.0; 2.0 | −19.0; −4.1 | −22.0; −22.0 | −42.0; 0.0 | −42.0; −10.0 |
| Percent change from baseline | | | | | | | | |
| Number (observed/imputed) | 33 (33/0) | 36 (35/1) | 34 (33/1) | 33 (33/0) | 4 (4/0) | 1 (1/0) | 4 (3/1) | 6 (5/1) |
| Mean (SD) | −35.69 (29.51) | −32.46 (26.63) | −44.07 (39.75) | −54.42 (33.80) | −30.69 (23.32) | −53.66 (NC) | −71.30 (48.04) | −70.71 (27.72) |
| Median | −40.48 | −27.86 | −40.89 | −50.00 | −29.91 | −53.66 | −92.59 | −73.90 |
| Q1; Q3 | −50.00; −13.89 | −48.70; −12.02 | −80.56; −7.69 | −82.14; −30.30 | −50.83; −10.56 | −53.66; −53.66 | −100.00; −42.59 | −95.24; −52.63 |
| Min; Max | −100.0; 43.8 | −100.0; 2.4 | −100.0; 44.8 | −100.0; 8.3 | −52.9; −10.0 | −53.7; −53.7 | −100.0; 0.0 | −100.0; −28.6 |
| Week 6 | | | | | | | | |
| Number (observed/imputed) | 32 (31/1) | 34 (33/1) | 34 (33/1) | 31 (31/0) | 4 (3/1) | 1 (1/0) | 5 (4/1) | 6 (5/1) |
| Mean (SD) | 19.67 (10.97) | 20.21 (10.42) | 17.02 (12.76) | 14.10 (11.23) | 27.25 (11.24) | 17.00 (NC) | 13.03 (15.45) | 7.00 (11.49) |
| Median | 21.00 | 18.25 | 16.50 | 14.00 | 26.00 | 17.00 | 5.83 | 3.00 |
| Q1; Q3 | 10.50; 28.00 | 14.00; 28.00 | 4.00; 26.83 | 4.67; 21.00 | 19.50; 35.00 | 17.00; 17.00 | 1.00; 23.33 | 0.00; 6.00 |
| Min; Max | 0.0; 42.0 | 0.0; 42.0 | 0.0; 42.0 | 0.0; 40.0 | 15.0; 42.0 | 17.0; 17.0 | 0.0; 35.0 | 0.0; 30.0 |
| Change from baseline | | | | | | | | |
| Number (observed/imputed) | 32 (31/1) | 34 (33/1) | 34 (33/1) | 31 (31/0) | 4 (3/1) | 1 (1/0) | 5 (4/1) | 6 (5/1) |
| Mean (SD) | −9.36 (11.33) | −11.79 (10.56) | −12.76 (11.42) | −15.94 (12.03) | −11.71 (8.21) | −24.00 (NC) | −18.97 (18.81) | −26.03 (14.05) |
| Median | −7.50 | −9.00 | −11.08 | −15.00 | −13.92 | −24.00 | −26.00 | −26.58 |
| Q1; Q3 | −14.40; −0.25 | −22.67; −2.67 | −24.00; −2.00 | −27.00; −4.83 | −17.00; −6.42 | −24.00; −24.00 | −35.00; 0.00 | −39.00; −12.00 |
| Min; Max | −40.0; 12.0 | −31.0; 3.3 | −32.0; 3.0 | −37.0; 3.5 | −19.0; 0.0 | −24.0; −24.0 | −36.2; 2.3 | −42.0; −10.0 |
| Percent change from baseline | | | | | | | | |
| Number (observed/imputed) | 32 (31/1) | 34 (33/1) | 34 (33/1) | 31 (31/0) | 4 (3/1) | 1 (1/0) | 5 (4/1) | 6 (5/1) |
| Mean (SD) | −29.60 (39.51) | −35.97 (30.17) | −43.89 (39.18) | −52.07 (35.76) | −31.44 (23.35) | −58.54 (NC) | −54.26 (54.98) | −78.40 (28.08) |
| Median | −26.59 | −37.30 | −38.06 | −47.83 | −34.95 | −58.54 | −86.11 | −89.66 |
| Q1; Q3 | −56.07; −1.39 | −58.12; −10.00 | −83.33; −8.33 | −81.33; −17.90 | −47.17; −15.71 | −58.54; −58.54 | −96.30; 0.00 | −100.00; −62.50 |
| Min; Max | −100.0; 75.0 | −100.0; 10.0 | −100.0; 13.3 | −100.0; 12.5 | −55.9; 0.0 | −58.5; −58.5 | −100.0; 11.1 | −100.0; −28.6 |
| Week 7 | | | | | | | | |
| Number (observed/imputed) | 33 (32/1) | 33 (31/2) | 34 (33/1) | 30 (30/0) | 4 (3/1) | 1 (1/0) | 5 (4/1) | 6 (5/1) |
| Mean (SD) | 19.78 (10.84) | 21.11 (10.03) | 15.55 (11.97) | 13.54 (9.34) | 30.35 (10.52) | 17.00 (NC) | 11.60 (15.79) | 12.17 (10.21) |
| Median | 21.00 | 18.20 | 14.88 | 14.00 | 31.30 | 17.00 | 2.00 | 11.50 |
| Q1; Q3 | 11.20; 28.00 | 14.00; 28.00 | 5.60; 26.00 | 6.00; 18.67 | 22.90; 37.80 | 17.00; 17.00 | 0.00; 21.00 | 6.00; 14.00 |
| Min; Max | 0.0; 42.0 | 0.0; 42.0 | 0.0; 42.0 | 0.0; 36.0 | 16.8; 42.0 | 17.0; 17.0 | 0.0; 35.0 | 0.0; 30.0 |
| Change from baseline | | | | | | | | |
| Number (observed/imputed) | 33 (32/1) | 33 (31/2) | 34 (33/1) | 30 (30/0) | 4 (3/1) | 1 (1/0) | 5 (4/1) | 6 (5/1) |
| Mean (SD) | −9.19 (10.67) | −10.92 (10.06) | −14.03 (11.20) | −16.56 (10.48) | −8.61 (7.11) | −24.00 (NC) | −20.40 (19.58) | −20.86 (14.89) |
| Median | 7.00 | −9.00 | −12.75 | −15.00 | −8.62 | −24.00 | −25.00 | −16.00 |
| Q1; Q3 | −14.00; −2.50 | −21.00; −3.00 | −22.00; −4.00 | −23.00; −9.00 | −13.60; −3.62 | −24.00; −24.00 | −35.00; 0.00 | −36.00; −8.17 |
| Min; Max | 40.0; 12.0 | −29.0; 3.3 | −39.0; 2.0 | −36.0; 3.5 | −17.2; 0.0 | −24.0; −24.0 | −42.0; 0.0 | −42.0; −7.0 |
| Percent change from baseline | | | | | | | | |
| Number (observed/imputed) | 33 (32/1) | 33 (31/2) | 34 (33/1) | 30 (30/0) | 4 (3/1) | 1 (1/0) | 5 (4/1) | 6 (5/1) |
| Mean (SD) | −29.76 (37.90) | −33.33 (28.49) | −48.14 (36.14) | −54.22 (30.59) | −23.49 (21.01) | −58.54 (NC) | −58.52 (53.51) | −58.95 (28.44) |
| Median | −21.88 | −36.11 | −48.28 | −52.50 | −21.68 | −58.54 | −92.59 | −51.29 |
| Q1; Q3 | −55.00; −10.00 | −57.14; −7.89 | −81.58; −22.22 | −76.00; −28.21 | −38.11; −8.86 | −58.54; −58.54 | −100.00; 0.00 | −85.71; −36.84 |
| Min; Max | −100.0; 75.0 | −100.0; 8.8 | −100.0; 6.9 | −100.0; 12.5 | −50.6; 0.0 | −58.5; −58.5 | −100.0; 0.0 | −100.0; −28.6 |
| Week 8 | | | | | | | | |
| Number (observed/imputed) | 33 (32/1) | 33 (30/3) | 33 (32/1) | 28 (28/0) | 4 (3/1) | 1 (1/0) | 5 (4/1) | 6 (5/1) |
| Mean (SD) | 19.31 (11.99) | 21.16 (10.52) | 13.98 (13.06) | 13.36 (9.81) | 23.52 (13.09) | 16.00 (NC) | 11.00 (15.67) | 7.50 (11.26) |
| Median | 18.00 | 21.00 | 10.00 | 14.00 | 20.20 | 16.00 | 1.00 | 3.00 |
| Q1; Q3 | 10.50; 29.40 | 14.00; 28.00 | 4.00; 22.00 | 4.00; 19.13 | 14.83; 32.20 | 16.00; 16.00 | 0.00; 19.00 | 2.00; 7.00 |
| Min; Max | 0.0; 42.0 | 0.0; 42.0 | 0.0; 42.0 | 0.0; 35.0 | 11.7; 42.0 | 16.0; 16.0 | 0.0; 35.0 | 0.0; 30.0 |
| Change from baseline | | | | | | | | |
| Number (observed/imputed) | 33 (32/1) | 33 (30/3) | 33 (32/1) | 28 (28/0) | 4 (3/1) | 1 (1/0) | 5 (4/1) | 6 (5/1) |
| Mean (SD) | −9.66 (11.77) | −10.63 (9.70) | −15.64 (11.76) | −16.89 (10.13) | −15.44 (10.42) | −25.00 (NC) | −21.00 (19.13) | −25.53 (12.52) |
| Median | −7.00 | −10.80 | −16.00 | −15.17 | −19.72 | −25.00 | −26.00 | −25.58 |
| Q1; Q3 | −18.00; −0.50 | −17.33; −2.00 | −24.00; −5.00 | −23.00; −10.00 | −21.67; −9.22 | −25.00; −25.00 | −35.00; −2.00 | −35.00; −13.00 |
| Min; Max | −37.0; 11.4 | −31.0; 3.3 | −42.0; 4.0 | −37.0; 1.0 | −22.3; 0.0 | −25.0; −25.0 | −42.0; 0.0 | 42.0; −12.0 |
| Percent change from baseline | | | | | | | | |
| Number (observed/imputed) | 33 (32/1) | 33 (30/3) | 33 (32/1) | 28 (28/0) | 4 (3/1) | 1 (1/0) | 5 (4/1) | 6 (5/1) |
| Mean (SD) | −32.03 (39.69) | −33.68 (30.26) | −54.37 (38.23) | −55.66 (31.25) | −41.17 (28.71) | −60.98 (NC) | −61.16 (51.62) | −78.96 (25.66) |
| Median | −23.81 | −31.82 | −64.29 | −55.83 | −49.49 | −60.98 | −96.30 | −84.90 |
| Q1; Q3 | −57.58; −1.28 | −54.17; −6.25 | −89.47; −15.38 | −82.48; −35.91 | −59.77; −22.57 | −60.98; −60.98 | −100.00; −9.52 | −94.12; −81.25 |
| Min; Max | −100.0; 63.3 | −100.0; 8.8 | −100.0; 16.0 | −100.0; 5.9 | −65.7; 0.0 | −61.0; −61.0 | −100.0; 0.0 | −100.0; −28.6 |
| Week 9 | | | | | | | | |
| Number (observed/imputed) | 32 (31/1) | 34 (31/3) | 29 (28/1) | 29 (29/0) | 4 (3/1) | 1 (1/0) | 5 (4/1) | 6 (5/1) |
| Mean (SD) | 20.65 (11.21) | 20.25 (10.25) | 14.64 (14.43) | 12.57 (9.72) | 21.35 (13.88) | 18.00 (NC) | 11.17 (15.78) | 7.89 (11.07) |
| Median | 20.00 | 19.33 | 11.00 | 12.00 | 15.40 | 18.00 | 1.00 | 4.00 |
| Q1; Q3 | 14.00; 28.00 | 14.00; 26.83 | 2.33; 22.00 | 4.00; 18.00 | 13.30; 29.40 | 18.00; 18.00 | 0.00; 19.83 | 2.33; 7.00 |
| Min; Max | 0.0; 42.0 | 0.0; 42.0 | 0.0; 42.0 | 0.0; 35.0 | 12.6; 42.0 | 18.0; 18.0 | 0.0; 35.0 | 0.0; 30.0 |
| Change from baseline | | | | | | | | |
| Number (observed/imputed) | 32 (31/1) | 34 (31/3) | 29 (28/1) | 29 (29/0) | 4 (3/1) | 1 (1/0) | 5 (4/1) | 6 (5/1) |
| Mean (SD) | −8.35 (11.00) | −11.75 (9.99) | −14.51 (11.46) | −17.88 (11.04) | −17.61 (12.62) | −23.00 (NC) | −20.83 (19.15) | −25.14 (12.48) |
| Median | −7.00 | −11.00 | −17.00 | −17.00 | −21.10 | −23.00 | −27.00 | −24.92 |
| Q1; Q3 | −14.00; −0.25 | −17.00; −5.00 | −22.00; −4.00 | −25.00; −10.00 | −26.62; −8.60 | −23.00; −23.00 | −35.00; −1.17 | −35.00; −12.00 |
| Min; Max | −35.0; 13.7 | −38.0; 3.3 | −35.7; 6.0 | −39.0; 4.0 | −28.2; 0.0 | −23.0; −23.0 | −41.0; 0.0 | −42.0; −12.0 |
| Percent change from baseline | | | | | | | | |
| Number (observed/imputed) | 32 (31/1) | 34 (31/3) | 29 (28/1) | 29 (29/0) | 4 (3/1) | 1 (1/0) | 5 (4/1) | 6 (5/1) |
| Mean (SD) | −26.90 (38.08) | −36.26 (27.83) | −53.27 (40.38) | −57.42 (32.58) | −45.96 (31.62) | −56.10 (NC) | −60.63 (52.86) | −77.44 (25.29) |
| Median | −22.88 | −32.58 | −59.77 | −60.00 | −57.35 | −56.10 | −97.62 | −85.78 |
| Q1; Q3 | −49.07; −1.39 | −51.72; −16.67 | −91.67; −15.38 | −82.14; −35.71 | −66.62; −25.29 | −56.10; −56.10 | −100.00; −5.56 | −89.47; −75.00 |
| Min; Max | −100.0; 56.3 | −100.0; 8.8 | −100.0; 24.0 | −100.0; 23.5 | −69.1; 0.0 | −56.1 ;−56.1 | −100.0; 0.0 | −100.0; −28.6 |
| Week 10 | | | | | | | | |
| Number (observed/imputed) | 33 (32/1) | 34 (31/3) | 28 (27/1) | 30 (30/0) | 4 (3/1) | 1 (1/0) | 5 (4/1) | 6 (4/2) |
| Mean (SD) | 19.73 (12.24) | 20.27 (10.69) | 15.22 (14.36) | 12.61 (9.89) | 16.78 (17.90) | 16.00 (NC) | 12.60 (14.98) | 7.83 (11.43) |
| Median | 19.83 | 17.00 | 13.50 | 12.83 | 11.40 | 16.00 | 4.00 | 4.00 |
| Q1; Q3 | 10.50; 28.00 | 14.00; 28.00 | 0.00; 23.50 | 4.00; 17.50 | 4.17; 29.40 | 16.00; 16.00 | 3.00; 21.00 | 0.00; 9.00 |
| Min; Max | 0.0; 42.0 | 0.0; 42.0 | 0.0; 42.0 | 0.0; 35.0 | 2.3; 42.0 | 16.0; 16.0 | 0.0; 35.0 | 0.0; 30.0 |
| Change from baseline | | | | | | | | |
| Number (observed/imputed) | 33 (32/1) | 34 (31/3) | 28 (27/1) | 30 (30/0) | 4 (3/1) | 1 (1/0) | 5 (4/1) | 6 (4/2) |
| Mean (SD) | −9.24 (11.57) | −11.73 (10.20) | −14.01 (11.59) | −17.75 (10.50) | −22.18 (17.32) | −25.00 (NC) | −19.40 (18.66) | −25.19 (15.27) |
| Median | −8.00 | −10.00 | −14.50 | −20.00 | −25.10 | −25.00 | −23.00 | −25.08 |
| Q1; Q3 | −16.33; 0.00 | −18.50; −3.00 | −24.00; −4.50 | −23.00; −10.00 | −35.75; −8.60 | −25.00; −25.00 | −35.00; 0.00 | 40.00; −12.00 |
| Min; Max | −35.0; 14.0 | −34.0; 3.3 | −32.0; 8.0 | −37.0; 0.5 | −38.5; 0.0 | 25.0; −25.0 | −39.0; 0.0 | −42.0; −7.0 |
| Percent change from baseline | | | | | | | | |
| Number (observed/imputed) | 33 (32/1) | 34 (31/3) | 28 (27/1) | 30 (30/0) | 4 (3/1) | 1 (1/0) | 5 (4/1) | 6 (4/2) |
| Mean (SD) | −30.98 (42.20) | −36.15 (29.79) | −50.88 (41.19) | −58.08 (31.82) | −57.37 (42.59) | −60.98 (NC) | −55.61 (51.03) | −73.42 (30.92) |
| Median | −33.33 | −36.70 | −51.79 | −59.49 | −67.60 | −60.98 | −85.19 | −84.09 |
| Q1; Q3 | −53.33; 0.00 | −62.07; −10.34 | −100.00; −17.06 | −84.00; −28.00 | −89.45; −25.29 | −60.98; −60.98 | −92.86; 0.00 | −100.00; −43.75 |
| Min; Max | −100.0; 56.3 | −100.0; 10.0 | −100.0; 32.0 | −100.0; 2.9 | −94.3; 0.0 | −61.0; −61.0 | −100.0; 0.0 | −100.0; −28.6 |
| Week 11 | | | | | | | | |
| Number (observed/imputed) | 33 (32/1) | 33 (30/3) | 29 (28/1) | 31 (31/0) | 4 (3/1) | 1 (1/0) | 5 (4/1) | 6 (4/2) |
| Mean (SD) | 19.89 (12.32) | 21.17 (11.16) | 15.06 (13.80) | 13.16 (10.54) | 17.58 (17.21) | 18.00 (NC) | 11.57 (15.45) | 7.08 (11.77) |
| Median | 21.00 | 20.00 | 11.20 | 13.00 | 13.00 | 18.00 | 2.00 | 1.75 |
| Q1; Q3 | 9.33; 28.00 | 14.00; 28.00 | 4.00; 22.00 | 4.00; 20.00 | 6.17; 29.00 | 18.00; 18.00 | 1.00; 19.83 | 0.00; 9.00 |
| Min; Max | 0.0; 42.0 | 0.0; 42.0 | 0.0; 42.0 | 0.0; 37.8 | 2.3; 42.0 | 18.0; 18.0 | 0.0; 35.0 | 0.0; 30.0 |
| Change from baseline | | | | | | | | |
| Number (observed/imputed) | 33 (32/1) | 33 (30/3) | 29 (28/1) | 31 (31/0) | 4 (3/1) | 1 (1/0) | 5 (4/1) | 6 (4/2) |
| Mean (SD) | −9.08 (11.91) | −11.28 (9.60) | −14.86 (11.83) | −17.13 (11.53) | −21.38 (16.53) | −23.00 (NC) | −20.43 (19.00) | −25.94 (15.41) |
| Median | −10.20 | −11.00 | −19.00 | −17.00 | −23.50 | −23.00 | −25.00 | −26.33 |
| Q1; Q3 | −18.00; 0.00 | −16.00; −3.00 | −23.80; −4.00 | −27.00; −8.00 | −33.75; −9.00 | −23.00; −23.00 | −35.00; −1.17 | −42.00; −12.00 |
| Min; Max | −35.0; 16.0 | −31.0; 3.3 | −38.0; 6.0 | −38.0; 9.8 | −38.5; 0.0 | −23.0; −23.0 | −41.0; 0.0 | −42.0; −7.0 |
| Percent change from baseline | | | | | | | | |
| Number (observed/imputed) | 33 (32/1) | 33 (30/3) | 29 (28/1) | 31 (31/0) | 4 (3/1) | 1 (1/0) | 5 (4/1) | 6 (4/2) |
| Mean (SD) | −29.69 (45.40) | −35.34 (30.18) | −51.82 (39.20) | −56.01 (34.22) | −55.40 (40.61) | −56.10 (NC) | −59.15 (51.57) | −76.09 (31.89) |
| Median | −33.33 | −34.19 | −57.14 | −60.00 | −63.65 | −56.10 | −92.59 | −92.11 |
| Q1; Q3 | −61.11; 0.00 | −50.00; −12.82 | −85.19; −21.05 | −88.89; −33.33 | −84.32; −26.47 | −56.10; −56.10 | −97.62; −5.56 | −100.00; −43.75 |
| Min; Max | −100.0; 75.0 | −100.0; 8.8 | −100.0; 24.0 | −100.0; 35.0 | −94.3; 0.0 | −56.1; −56.1 | −100.0; 0.0 | −100.0; −28.6 |
| Week 12 | | | | | | | | |
| Number (observed/imputed) | 32 (31/1) | 30 (27/3) | 26 (24/2) | 28 (27/1) | 3 (2/1) | 1 (1/0) | 4 (3/1) | 5 (3/2) |
| Mean (SD) | 19.32 (13.04) | 22.00 (11.07) | 15.61 (13.91) | 11.00 (9.90) | 18.67 (21.39) | 16.00 (NC) | 8.75 (17.50) | 7.80 (13.01) |
| Median | 17.75 | 22.87 | 14.00 | 12.25 | 14.00 | 16.00 | 0.00 | 0.00 |
| Q1; Q3 | 8.17; 28.50 | 14.00; 28.00 | 2.00; 26.00 | 1.00; 17.15 | 0.00; 42.00 | 16.00; 16.00 | 0.00; 17.50 | 0.00; 9.00 |
| Min; Max | 0.0; 42.0 | 0.0; 42.0 | 0.0; 42.0 | 0.0; 35.0 | 0.0; 42.0 | 16.0; 16.0 | 0.0; 35.0 | 0.0; 30.0 |
| Change from baseline | | | | | | | | |
| Number (observed/imputed) | 32 (31/1) | 30 (27/3) | 26 (24/2) | 28 (27/1) | 3 (2/1) | 1 (1/0) | 4 (3/1) | 5 (3/2) |
| Mean (SD) | −9.71 (13.20) | −10.57 (9.36) | −14.49 (12.08) | −19.04 (11.40) | −21.94 (20.59) | −25.00 (NC) | −26.00 (18.38) | −27.40 (16.76) |
| Median | −8.00 | −8.70 | −12.80 | −18.92 | −25.00 | −25.00 | −31.00 | −34.00 |
| Q1; Q3 | −18.50; −0.50 | −17.33; −3.67 | −22.00; −4.00 | −28.50; −8.75 | −40.83; 0.00 | −25.00; −25.00 | −38.50; −13.50 | −42.00; −12.00 |
| Min; Max | −35.0; 20.5 | −31.0; 3.3 | −40.8; 1.0 | −42.0; −1.0 | −40.8; 0.0 | −25.0; −25.0 | −42.0; 0.0 | −42.0; −7.0 |
| Percent change from baseline | | | | | | | | |
| Number (observed/imputed) | 32 (31/1) | 30 (27/3) | 26 (24/2) | 28 (27/1) | 3 (2/1) | 1 (1/0) | 4 (3/1) | 5 (3/2) |
| Mean (SD) | −31.31 (48.12) | −33.23 (28.33) | −50.20 (37.89) | −62.32 (31.89) | −54.70 (50.66) | −60.98 (NC) | −75.00 (50.00) | −74.46 (35.38) |
| Median | −26.76 | −29.67 | −47.76 | −60.00 | −64.10 | −60.98 | −100.00 | −100.00 |
| Q1; Q3 | −68.36; −2.03 | −48.15; −12.50 | −91.67; −16.67 | −97.14; −34.52 | −100.00; 0.00 | −60.98; −60.98 | −100.00; −50.00 | −100.00; −43.75 |
| Min; Max | −100.0; 113.9 | −100.0; 8.8 | −100.0; 2.9 | −100.0; −2.8 | −100.0; 0.0 | −61.0; −61.0 | −100.0; 0.0 | −100.0; −28.6 |
| End of Study | | | | | | | | |
| Number (observed/imputed) | 5 (3/2) | 7 (3/4) | 5 (3/2) | 5 (3/2) | 0 (0/0) | 0 (0/0) | 0 (0/0) | 2 (0/2) |
| Mean (SD) | 22.10 (13.19) | 11.70 (16.82) | 15.60 (17.52) | 21.40 (11.74) | | | | 15.58 (9.31) |
| Median | 24.50 | 5.00 | 14.00 | 20.00 | | | | 15.58 |
| Q1; Q3 | 23.00; 28.00 | 0.00; 30.33 | 0.00; 22.00 | 14.00; 30.00 | | | | 9.00; 22.17 |
| Min; Max | 0.0; 35.0 | 0.0; 41.0 | 0.0; 42.0 | 7.0; 36.0 | | | | 9.0; 22.2 |
| Change from baseline | | | | | | | | |
| Number (observed/imputed) | 5 (3/2) | 7 (3/4) | 5 (3/2) | 5 (3/2) | 0 (0/0) | 0 (0/0) | 0 (0/0) | 2 (0/2) |
| Mean (SD) | −7.30 (10.21) | −21.44 (16.26) | −14.40 (13.79) | −11.40 (6.62) | | | | −3.50 (4.95) |
| Median | −11.00 | −28.00 | −13.00 | −14.00 | | | | −3.50 |
| Q1; Q3 | −14.00; 0.00 | −36.40; 0.00 | −28.00; −2.00 | −14.00; −12.00 | | | | −7.00; 0.00 |
| Min; Max | −18.0; 6.5 | −38.0; 0.3 | −29.0; 0.0 | −17.0; 0.0 | | | | −7.0; 0.0 |
| Percent change from baseline | | | | | | | | |
| Number (observed/imputed) | 5 (3/2) | 7 (3/4) | 5 (3/2) | 5 (3/2) | 0 (0/0) | 0 (0/0) | 0 (0/0) | 2 (0/2) |
| Mean (SD) | −25.92 (50.26) | −66.12 (46.34) | −51.30 (48.04) | −38.24 (25.31) | | | | −21.88 (30.94) |
| Median | −32.35 | −86.67 | −48.15 | −45.95 | | | | −21.88 |
| Q1; Q3 | −33.33; 0.00 | −100.00; 0.00 | −100.00; −8.33 | −50.00; −28.57 | | | | −43.75; 0.00 |
| Min; Max | −100.0; 36.1 | −100.0; 1.1 | −100.0; 0.0 | −66.7; 0.0 | | | | −43.8; 0.0 |

Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF.

TABLE 18
Change and Percent Change from Baseline in UAS7 over Time up to Week 12 (ITT population)

| | | | | Change | | | % Change | | |
|---|---|---|---|---|---|---|---|---|---|
| | | | Post- | from | Change difference for | | from | % Change difference for | |
| | | Baseline | baseline | baseline | rilzabrutinib vs. placebo | | baseline | rilzabrutinib vs. placebo | |
| | | Mean | Mean | LS Mean | LS Mean | | LS Mean | LS Mean | |
| | n | (SD) | (SD) | (SE)^{a} | (95% CI)^{a} | p-valueª | (SE)^{a} | (95% CI)^{a} | p-valueª |
| Week 1 | | | | | | | | | |
| Rilzabrutinib 400 mg TID | 41 | 29.88 (8.57) | 16.95 (10.13) | −12.72 | −8.89 | <.0001 | −40.18 | −29.94 | <.0001 |
| (N = 41) | | | | (1.45) | (−13.65, −4.13) | | (5.17) | (−43.87, −16.00) | |
| Rilzabrutinib 400 mg BID | 41 | 30.21 (7.19) | 19.98 (11.35) | −10.01 | −6.18 | 0.0024 | −34.18 | −23.93 | 0.0007 |
| (N = 41) | | | | (1.48) | (−10.92, −1.44) | | (5.22) | (−37.73, −10.14) | |
| Rilzabrutinib 400 mg QPM | 38 | 31.37 (7.32) | 23.84 (8.66) | −7.44 | −3.61 | 0.0785 | −22.47 | −12.22 | 0.0887 |
| (N = 38) | | | | (1.50) | (−8.44, 1.23) | | (5.29) | (−26.29, 1.85) | |
| Placebo (N = 40) | 40 | 29.97 (8.56) | 26.37 (10.35) | −3.83 | | | −10.25 | | |
| | | | | (1.48) | | | (5.23) | | |
| Week 2 | | | | | | | | | |
| Rilzabrutinib 400 mg TID | 41 | 29.88 (8.57) | 14.58 (10.74) | −15.06 | −8.72 | <.0001 | −48.69 | −29.83 | <.0001 |
| (N = 41) | | | | (1.50) | (−12.78, −4.67) | | (5.12) | (−43.69, −15.96) | |
| Rilzabrutinib 400 mg BID | 40 | 30.21 (7.19) | 19.50 (11.13) | −10.82 | −4.49 | 0.0296 | −36.13 | −17.26 | 0.0135 |
| (N = 41) | | | | (1.53) | (−8.54, −0.44) | | (5.18) | (−30.96, −3.56) | |
| Rilzabrutinib 400 mg QPM | 38 | 31.37 (7.32) | 23.40 (8.85) | −8.14 | −1.80 | 0.3911 | −25.19 | −6.32 | 0.3751 |
| (N = 38) | | | | (1.55) | (−5.93, 2.32) | | (5.23) | (−20.30, 7.65) | |
| Placebo (N = 40) | 39 | 29.97 (8.56) | 23.88 (10.51) | −6.33 | | | −18.87 | | |
| | | | | (1.53) | | | (5.20) | | |
| Week 3 | | | | | | | | | |
| Rilzabrutinib 400 mg TID | 41 | 29.88 (8.57) | 14.42 (9.77) | −15.15 | −7.56 | 0.0005 | −49.95 | −28.32 | <.0001 |
| (N = 41) | | | | (1.57) | (−11.83, −3.30) | | (5.25) | (−42.50, −14.13) | |
| Rilzabrutinib 400 mg BID | 41 | 30.21 (7.19) | 17.63 (12.02) | −12.33 | −4.74 | 0.0290 | −39.97 | −18.34 | 0.0105 |
| (N = 41) | | | | (1.60) | (−8.99, −0.49) | | (5.30) | (−32.39, −4.29) | |
| Rilzabrutinib 400 mg QPM | 38 | 31.37 (7.32) | 21.93 (8.89) | −9.50 | −1.91 | 0.3889 | −29.55 | −7.92 | 0.2790 |
| (N = 38) | | | | (1.63) | (−6.25, 2.43) | | (5.37) | (−22.26, 6.42) | |
| Placebo (N = 40) | 39 | 29.97 (8.56) | 22.67 (11.08) | −7.59 | | | −21.63 | | |
| | | | | (1.62) | | | (5.34) | | |
| Week 4 | | | | | | | | | |
| Rilzabrutinib 400 mg TID | 38 | 29.88 (8.57) | 13.93 (9.77) | −14.78 | −6.71 | 0.0030 | −48.94 | −25.68 | 0.0007 |
| (N = 41) | | | | (1.64) | (−11.14, −2.27) | | (5.48) | (−40.58, −10.78) | |
| Rilzabrutinib 400 mg BID | 38 | 30.21 (7.19) | 15.62 (12.20) | −13.41 | −5.34 | 0.0177 | −44.02 | −20.76 | 0.0056 |
| (N = 41) | | | | (1.67) | (−9.76, −0.93) | | (5.54) | (−35.44, −6.08) | |
| Rilzabrutinib 400 mg QPM | 38 | 31.37 (7.32) | 21.82 (8.73) | −9.55 | −1.48 | 0.5181 | −29.23 | −5.97 | 0.4329 |
| (N = 38) | | | | (1.68) | (−5.98, 3.01) | | (5.56) | (−20.90, 8.96) | |
| Placebo (N = 40) | 38 | 29.97 (8.56) | 21.85 (11.96) | −8.07 | | | −23.26 | | |
| | | | | (1.68) | | | (5.57) | | |
| Week 5 | | | | | | | | | |
| Rilzabrutinib 400 mg TID | 39 | 29.88 (8.57) | 12.72 (10.63) | −16.68 | −6.47 | 0.0043 | −54.01 | −22.21 | 0.0024 |
| (N = 41) | | | | (1.65) | (−10.92, −2.03) | | (5.30) | (−36.58, −7.85) | |
| Rilzabrutinib 400 mg BID | 38 | 30.21 (7.19) | 16.44 (13.70) | −12.81 | −2.61 | 0.2503 | −42.67 | −10.88 | 0.1338 |
| (N = 41) | | | | (1.68) | (−7.06, 1.84) | | (5.37) | (−25.10, 3.34) | |
| Rilzabrutinib 400 mg QPM | 37 | 31.37 (7.32) | 20.58 (9.05) | −10.47 | −0.26 | 0.9091 | −32.52 | −0.72 | 0.9222 |
| (N = 38) | | | | (1.69) | (−4.77, 4.25) | | (5.40) | (−15.19, 13.75) | |
| Placebo (N = 40) | 37 | 29.97 (8.56) | 19.30 (9.71) | −10.20 | | | −31.80 | | |
| | | | | (1.68) | | | (5.40) | | |
| Week 6 | | | | | | | | | |
| Rilzabrutinib 400 mg TID | 37 | 29.88 (8.57) | 12.95 (11.42) | −16.68 | −7.54 | 0.0022 | −54.12 | −27.22 | 0.0009 |
| (N = 41) | | | | (1.78) | (−12.35, −2.72) | | (5.89) | (−43.32, −11.13) | |
| Rilzabrutinib 400 mg BID | 39 | 30.21 (7.19) | 16.51 (12.98) | −12.78 | −3.64 | 0.1341 | −41.66 | −14.76 | 0.0669 |
| (N = 41) | | | | (1.79) | (−8.40, 1.12) | | (5.92) | (−30.55, 1.03) | |
| Rilzabrutinib 400 mg QPM | 35 | 31.37 (7.32) | 20.12 (10.28) | −11.02 | −1.88 | 0.4515 | −34.14 | −7.24 | 0.3795 |
| (N = 38) | | | | (1.82) | (−6.78, 3.02) | | (6.02) | (−23.38, 8.91) | |
| Placebo (N = 40) | 36 | 29.97 (8.56) | 20.51 (11.10) | −9.14 | | | −26.90 | | |
| | | | | (1.82) | | | (6.05) | | |
| Week 7 | | | | | | | | | |
| Rilzabrutinib 400 mg TID | 36 | 29.88 (8.57) | 13.31 (9.35) | −16.36 | −7.30 | 0.0017 | −52.94 | −25.60 | 0.0008 |
| (N = 41) | | | | (1.70) | (−11.86, −2.74) | | (5.57) | (−40.60, −10.59) | |
| Rilzabrutinib 400 mg BID | 39 | 30.21 (7.19) | 15.04 (12.35) | −14.56 | −5.50 | 0.0170 | −47.80 | −20.46 | 0.0067 |
| (N = 41) | | | | (1.71) | (−10.02, −0.98) | | (5.58) | (−35.24, −5.68) | |
| Rilzabrutinib 400 mg QPM | 34 | 31.37 (7.32) | 20.98 (9.91) | −10.95 | −1.89 | 0.4271 | −34.50 | −7.16 | 0.3600 |
| (N = 38) | | | | (1.74) | (−6.55, 2.77) | | (5.71) | (−22.48, 8.17) | |
| Placebo (N = 40) | 37 | 29.97 (8.56) | 20.92 (11.17) | −9.06 | | | −27.34 | | |
| | | | | (1.72) | | | (5.65) | | |
| Week 8 | | | | | | | | | |
| Rilzabrutinib 400 mg TID | 34 | 29.88 (8.57) | 12.33 (10.15) | −16.52 | −6.17 | 0.0132 | −54.62 | −22.78 | 0.0049 |
| (N = 41) | | | | (1.81) | (−11.06, −1.29) | | (5.88) | (−38.64, −6.92) | |
| Rilzabrutinib 400 mg BID | 38 | 30.21 (7.19) | 13.59 (13.23) | −15.96 | −5.61 | 0.0205 | −53.24 | −21.40 | 0.0071 |
| (N = 41) | | | | (1.78) | (−10.36, −0.86) | | (5.86) | (−36.98, −5.82) | |
| Rilzabrutinib 400 mg QPM | 34 | 31.37 (7.32) | 21.01 (10.40) | −10.88 | −0.53 | 0.8324 | −35.22 | −3.38 | 0.6780 |
| (N = 38) | | | | (1.83) | (−5.43, 4.37) | | (5.96) | (−19.31, 12.56) | |
| Placebo (N = 40) | 37 | 29.97 (8.56) | 19.76 (11.99) | −10.35 | | | −31.84 | | |
| | | | | (1.81) | | | (5.90) | | |
| Week 9 | | | | | | | | | |
| Rilzabrutinib 400 mg TID | 35 | 29.88 (8.57) | 11.76 (9.95) | −16.69 | −7.09 | 0.0050 | −54.53 | −26.48 | 0.0014 |
| (N = 41) | | | | (1.82) | (−12.04, −2.14) | | (6.02) | (−42.71, −10.25) | |
| Rilzabrutinib 400 mg BID | 34 | 30.21 (7.19) | 14.13 (14.43) | −15.45 | −5.85 | 0.0199 | −52.21 | −24.16 | 0.0027 |
| (N = 41) | | | | (1.85) | (−10.77, −0.92) | | (5.98) | (−39.96, −8.36) | |
| Rilzabrutinib 400 mg QPM | 35 | 31.37 (7.32) | 20.18 (10.10) | −11.33 | −1.73 | 0.4925 | −36.53 | −8.48 | 0.3055 |
| (N = 38) | | | | (1.85) | (−6.68, 3.21) | | (6.04) | (−24.69, 7.74) | |
| Placebo (N = 40) | 36 | 29.97 (8.56) | 20.73 (11.30) | −9.60 | | | −28.05 | | |
| | | | | (1.85) | | | (5.98) | | |
| Week 10 | | | | | | | | | |
| Rilzabrutinib 400 mg TID | 36 | 29.88 (8.57) | 11.82 (10.15) | −17.20 | −6.30 | 0.0153 | −56.75 | −23.22 | 0.0057 |
| (N = 41) | | | | (1.88) | (−11.39, −1.21) | | (6.13) | (−39.70, −6.74) | |
| Rilzabrutinib 400 mg BID | 33 | 30.21 (7.19) | 14.82 (14.24) | −15.36 | −4.46 | 0.0874 | −51.13 | −17.60 | 0.0359 |
| (N = 41) | | | | (1.94) | (−9.57, 0.65) | | (6.28) | (−34.05, −1.16) | |
| Rilzabrutinib 400 mg QPM | 35 | 31.37 (7.32) | 20.15 (10.55) | −11.66 | −0.76 | 0.7746 | −37.35 | −3.82 | 0.6555 |
| (N = 38) | | | | (1.94) | (−5.94, 4.43) | | (6.27) | (−20.62, 12.97) | |
| Placebo (N = 40) | 37 | 29.97 (8.56) | 19.41 (12.68) | −10.90 | | | −33.52 | | |
| | | | | (1.92) | | | (6.21) | | |
| Week 11 | | | | | | | | | |
| Rilzabrutinib 400 mg TID | 37 | 29.88 (8.57) | 12.17 (10.81) | −17.15 | −6.74 | 0.0087 | −55.80 | −25.03 | 0.0040 |
| (N = 41) | | | | (1.87) | (−11.78, −1.71) | | (6.27) | (−42.07, −7.99) | |
| Rilzabrutinib 400 mg BID | 34 | 30.21 (7.19) | 14.55 (13.86) | −14.95 | −4.55 | 0.0761 | −49.87 | −19.11 | 0.0267 |
| (N = 41) | | | | (1.92) | (−9.58, 0.48) | | (6.37) | (−36.01, −2.21) | |
| Rilzabrutinib 400 mg QPM | 34 | 31.37 (7.32) | 21.08 (11.00) | −10.92 | −0.52 | 0.8440 | −36.39 | −5.62 | 0.5185 |
| (N = 38) | | | | (1.93) | (−5.67, 4.64) | | (6.38) | (−22.69, 11.45) | |
| Placebo (N = 40) | 37 | 29.97 (8.56) | 19.64 (12.65) | −10.40 | | | −30.76 | | |
| | | | | (1.90) | | | (6.34) | | |
| Week 12 | | | | | | | | | |
| Rilzabrutinib 400 mg TID | 33 | 29.88 (8.57) | 10.52 (10.26) | −17.95 | −6.76 | 0.0116 | −59.16 | −26.23 | 0.0030 |
| (N = 41) | | | | (1.95) | (−12.01, −1.51) | | (6.41) | (−43.53, −8.93) | |
| Rilzabrutinib 400 mg BID | 30 | 30.21 (7.19) | 14.70 (14.29) | −14.82 | −3.62 | 0.1763 | −49.34 | −16.41 | 0.0660 |
| (N = 41) | | | | (1.98) | (−8.87, 1.63) | | (6.68) | (−33.90, 1.08) | |
| Rilzabrutinib 400 mg QPM | 31 | 31.37 (7.32) | 21.80 (10.94) | −10.22 | 0.97 | 0.7230 | −35.03 | −2.09 | 0.8167 |
| (N = 38) | | | | (2.01) | (−4.40, 6.34) | | (6.66) | (−19.80, 15.61) | |
| Placebo (N = 40) | 35 | 29.97 (8.56) | 19.27 (13.49) | −11.20 | | | −32.93 | | |
| | | | | (1.98) | | | (6.46) | | |

^{a}Each of the imputed complete data were analyzed by fitting an ANCOVA model with the corresponding baseline value, intervention group and regions as covariates.

TABLE 19
Change and Percent Change from Baseline in UAS7 over Time up to Week 12 (Omalizumab-Naïve Population)

| | | | | Change | | | % Change | | |
|---|---|---|---|---|---|---|---|---|---|
| | | | Post- | from | Change difference for | | from | % Change difference for | |
| | | Baseline | baseline | baseline | rilzabrutinib vs. placebo | | baseline | rilzabrutinib vs. placebo | |
| | | Mean | Mean | LS Mean | LS Mean | | LS Mean | LS Mean | |
| | n | (SD) | (SD) | (SE)^{a} | (95% CI)ª | p-valueª | (SE)^{a} | (95% CI)ª | p-valueª |
| Week 1 | | | | | | | | | |
| Rilzabrutinib 400 mg TID | 35 | 29.34 (8.08) | 17.77 (10.26) | −11.44 (1.56) | −7.89 | 0.0003 | −36.60 (5.66) | −27.13 | 0.0004 |
| (N = 35) | | | | | (−12.98, −2.81) | | | (−42.27, −11.98) | |
| Rilzabrutinib 400 mg BID | 35 | 29.73 (7.11) | 19.77 (11.32) | −9.79 (1.58) | −6.24 | 0.0040 | −34.48 (5.64) | −25.01 | 0.0010 |
| (N = 35) | | | | | (−11.31, −1.18) | | | (−39.96, −10.06) | |
| Rilzabrutinib 400 mg QPM | 37 | 31.11 (7.24) | 23.81 (8.78) | −7.06 (1.52) | −3.51 | 0.0983 | −21.67 (5.45) | −12.19 | 0.1062 |
| (N = 37) | | | | | (−8.52, 1.50) | | | (−26.98, 2.60) | |
| Placebo (N = 36) | 36 | 28.97 (8.39) | 25.80 (10.44) | −3.55 (1.56) | | | −9.48 (5.57) | | |
| Week 2 | | | | | | | | | |
| Rilzabrutinib 400 mg TID | 35 | 29.34 (8.08) | 15.65 (10.66) | −13.63 (1.56) | −7.73 | 0.0003 | −44.90 (5.51) | −27.13 | 0.0003 |
| (N = 35) | | | | | (−11.91, −3.54) | | | (−41.93, −12.34) | |
| Rilzabrutinib 400 mg BID | 34 | 29.73 (7.11) | 19.59 (10.86) | −10.44 (1.57) | −4.55 | 0.0327 | −36.05 (5.50) | −18.28 | 0.0140 |
| (N = 35) | | | | | (−8.72, −0.37) | | | (−32.86, −3.71) | |
| Rilzabrutinib 400 mg QPM | 37 | 31.11 (7.24) | 23.44 (8.97) | −7.71 (1.52) | −1.81 | 0.3884 | −24.37 (5.29) | −6.60 | 0.3693 |
| (N = 37) | | | | | (−5.93, 2.31) | | | (−21.02, 7.82) | |
| Placebo (N = 36) | 35 | 28.97 (8.39) | 23.49 (10.05) | −5.90 (1.55) | | | −17.76 (5.44) | | |
| Week 3 | | | | | | | | | |
| Rilzabrutinib 400 mg TID | 35 | 29.34 (8.08) | 15.37 (9.87) | −13.83 (1.65) | −6.90 | 0.0024 | −47.14 (5.69) | −27.19 | 0.0005 |
| (N = 35) | | | | | (−11.35, −2.45) | | | (−42.43, −11.95) | |
| Rilzabrutinib 400 mg BID | 35 | 29.73 (7.11) | 17.71 (11.91) | −11.96 (1.67) | −5.04 | 0.0259 | −40.11 (5.66) | −20.16 | 0.0087 |
| (N = 35) | | | | | (−9.48, −0.61) | | | (−35.23, −5.10) | |
| Rilzabrutinib 400 mg QPM | 37 | 31.11 (7.24) | 22.01 (9.00) | −9.10 (1.61) | −2.17 | 0.3319 | −28.93 (5.48) | −8.98 | 0.2377 |
| (N = 37) | | | | | (−6.56, 2.22) | | | (−23.89, 5.93) | |
| Placebo (N = 36) | 35 | 28.97 (8.39) | 22.60 (10.89) | −6.92 (1.66) | | | −19.95 (5.62) | | |
| Week 4 | | | | | | | | | |
| Rilzabrutinib 400 mg TID | 32 | 29.34 (8.08) | 14.65 (9.61) | −13.66 (1.72) | −6.60 | 0.0052 | −46.75 (5.92) | −26.33 | 0.0012 |
| (N = 35) | | | | | (−11.22, −1.97) | | | (−42.23, −10.43) | |
| Rilzabrutinib 400 mg BID | 33 | 29.73 (7.11) | 15.92 (11.71) | −12.89 (1.73) | −5.82 | 0.0132 | −43.62 (5.88) | −23.20 | 0.0036 |
| (N = 35) | | | | | (−10.43, −1.22) | | | (−38.83, −7.56) | |
| Rilzabrutinib 400 mg QPM | 37 | 31.11 (7.24) | 21.92 (8.82) | −9.14 (1.66) | −2.08 | 0.3699 | −28.63 (5.66) | −8.22 | 0.2988 |
| (N = 37) | | | | | (−6.63, 2.47) | | | (−23.72, 7.28) | |
| Placebo (N = 36) | 34 | 28.97 (8.39) | 22.09 (12.20) | −7.06 (1.72) | | | −20.42 (5.86) | | |
| Week 5 | | | | | | | | | |
| Rilzabrutinib 400 mg TID | 33 | 29.34 (8.08) | 13.41 (10.60) | −15.54 (1.72) | −5.10 | 0.0314 | −52.02 (5.69) | −19.17 | 0.0146 |
| (N = 35) | | | | | (−9.74, −0.45) | | | (−34.55, −3.79) | |
| Rilzabrutinib 400 mg BID | 34 | 29.73 (7.11) | 17.22 (13.36) | −12.04 (1.72) | −1.60 | 0.4942 | −41.58 (5.68) | −8.73 | 0.2620 |
| (N = 35) | | | | | (−6.18, 2.98) | | | (−23.98, 6.52) | |
| Rilzabrutinib 400 mg QPM | 36 | 31.11 (7.24) | 20.63 (9.18) | −10.08 (1.67) | 0.36 | 0.8770 | −31.92 (5.50) | 0.93 | 0.9046 |
| (N = 37) | | | | | (−4.19, 4.90) | | | (−14.21, 16.06) | |
| Placebo (N = 36) | 33 | 28.97 (8.39) | 18.31 (9.22) | −10.44 (1.72) | | | −32.85 (5.72) | | |
| Week 6 | | | | | | | | | |
| Rilzabrutinib 400 mg TID | 31 | 29.34 (8.08) | 14.10 (11.23) | −15.12 (1.90) | −5.94 | 0.0218 | −50.65 (6.35) | −24.10 | 0.0058 |
| (N = 35) | | | | | (−11.02, −0.87) | | | (−41.21, −7.00) | |
| Rilzabrutinib 400 mg BID | 34 | 29.73 (7.11) | 17.02 (12.76) | −12.21 (1.87) | −3.03 | 0.2347 | −41.23 (6.32) | −14.68 | 0.0881 |
| (N = 35) | | | | | (−8.03, 1.97) | | | (−31.55, 2.19) | |
| Rilzabrutinib 400 mg QPM | 34 | 31.11 (7.24) | 20.21 (10.42) | −10.46 (1.82) | −1.28 | 0.6143 | −33.30 (6.14) | −6.75 | 0.4286 |
| (N = 37) | | | | | (−6.26, 3.70) | | | (−23.47, 9.97) | |
| Placebo (N = 36) | 32 | 28.97 (8.39) | 19.67 (10.97) | −9.18 (1.88) | | | −26.55 (6.32) | | |
| Week 7 | | | | | | | | | |
| Rilzabrutinib 400 mg TID | 30 | 29.34 (8.08) | 13.54 (9.34) | −15.80 (1.80) | −6.37 | 0.0096 | −52.91 (6.13) | −24.35 | 0.0034 |
| (N = 35) | | | | | (−11.19, −1.55) | | | (−40.66, −8.04) | |
| Rilzabrutinib 400 mg BID | 34 | 29.73 (7.11) | 15.55 (11.97) | −13.94 (1.77) | −4.51 | 0.0617 | −47.01 (5.97) | −18.46 | 0.0236 |
| (N = 35) | | | | | (−9.23, 0.22) | | | (−34.43, −2.48) | |
| Rilzabrutinib 400 mg QPM | 33 | 31.11 (7.24) | 21.11 (10.03) | −10.55 (1.74) | −1.12 | 0.6443 | −34.23 (5.85) | −5.67 | 0.4852 |
| (N = 37) | | | | | (−5.86, 3.63) | | | (−21.60, 10.26) | |
| Placebo (N = 36) | 33 | 28.97 (8.39) | 19.78 (10.84) | −9.43 (1.77) | | | −28.56 (5.99) | | |
| Week 8 | | | | | | | | | |
| Rilzabrutinib 400 mg TID | 28 | 29.34 (8.08) | 13.36 (9.81) | −15.04 (1.96) | −5.11 | 0.0532 | −50.90 (6.49) | −20.07 | 0.0222 |
| (N = 35) | | | | | (−10.30, 0.07) | | | (−37.26, −2.87) | |
| Rilzabrutinib 400 mg BID | 33 | 29.73 (7.11) | 13.98 (13.06) | −15.47 (1.87) | −5.54 | 0.0299 | −53.47 (6.30) | −22.63 | 0.0083 |
| (N = 35) | | | | | (−10.53, −0.54) | | | (−39.45, −5.82) | |
| Rilzabrutinib 400 mg QPM | 33 | 31.11 (7.24) | 21.16 (10.52) | −10.40 (1.83) | −0.47 | 0.8534 | −34.61 (6.22) | −3.78 | 0.6578 |
| (N = 37) | | | | | (−5.46, 4.52) | | | (−20.49, 12.94) | |
| Placebo (N = 36) | 33 | 28.97 (8.39) | 19.31 (11.99) | −9.93 (1.87) | | | −30.84 (6.24) | | |
| Week 9 | | | | | | | | | |
| Rilzabrutinib 400 mg TID | 29 | 29.34 (8.08) | 12.57 (9.72) | −15.75 (1.93) | −7.07 | 0.0067 | −52.54 (6.34) | −25.99 | 0.0028 |
| (N = 35) | | | | | (−12.19, −1.96) | | | (−43.04, −8.94) | |
| Rilzabrutinib 400 mg BID | 29 | 29.73 (7.11) | 14.64 (14.43) | −14.84 (1.91) | −6.16 | 0.0173 | −52.38 (6.34) | −25.82 | 0.0026 |
| (N = 35) | | | | | (−11.24, −1.09) | | | (−42.66, −8.98) | |
| Rilzabrutinib 400 mg QPM | 34 | 31.11 (7.24) | 20.25 (10.25) | −11.03 (1.86) | −2.36 | 0.3609 | −35.89 (6.15) | −9.34 | 0.2740 |
| (N = 37) | | | | | (−7.42, 2.70) | | | (−26.06, 7.39) | |
| Placebo (N = 36) | 32 | 28.97 (8.39) | 20.65 (11.21) | −8.67 (1.90) | | | −26.56 (6.30) | | |
| Week 10 | | | | | | | | | |
| Rilzabrutinib 400 mg TID | 30 | 29.34 (8.08) | 12.61 (9.89) | −16.20 (1.97) | −6.54 | 0.0149 | −54.80 (6.67) | −24.83 | 0.0069 |
| (N = 35) | | | | | (−11.80, −1.28) | | | (−42.85, −6.81) | |
| Rilzabrutinib 400 mg BID | 28 | 29.73 (7.11) | 15.22 (14.36) | −14.89 (2.00) | −5.22 | 0.0523 | −51.13 (6.68) | −21.17 | 0.0190 |
| (N = 35) | | | | | (−10.50, 0.05) | | | (−38.85, −3.48) | |
| Rilzabrutinib 400 mg QPM | 34 | 31.11 (7.24) | 20.27 (10.69) | −11.25 (1.92) | −1.58 | 0.5492 | −37.03 (6.40) | −7.06 | 0.4332 |
| (N = 37) | | | | | (−6.76, 3.60) | | | (−24.72, 10.60) | |
| Placebo (N = 36) | 33 | 28.97 (8.39) | 19.73 (12.24) | −9.66 (1.95) | | | −29.97 (6.65) | | |
| Week 11 | | | | | | | | | |
| Rilzabrutinib 400 mg TID | 31 | 29.34 (8.08) | 13.16 (10.54) | −15.86 (1.96) | −6.58 | 0.0143 | −53.47 (6.81) | −25.26 | 0.0071 |
| (N = 35) | | | | | (−11.85, −1.31) | | | (−43.63, −6.89) | |
| Rilzabrutinib 400 mg BID | 29 | 29.73 (7.11) | 15.06 (13.80) | −14.56 (1.97) | −5.29 | 0.0479 | −49.86 (6.78) | −21.65 | 0.0193 |
| (N = 35) | | | | | (−10.53, −0.05) | | | (−39.78, −3.52) | |
| Rilzabrutinib 400 mg QPM | 33 | 31.11 (7.24) | 21.17 (11.16) | −10.63 (1.93) | −1.36 | 0.6091 | −35.67 (6.65) | −7.47 | 0.4145 |
| (N = 37) | | | | | (−6.58, 3.86) | | | (−25.41, 10.47) | |
| Placebo (N = 36) | 33 | 28.97 (8.39) | 19.89 (12.32) | −9.27 (1.94) | | | −28.20 (6.73) | | |
| Week 12 | | | | | | | | | |
| Rilzabrutinib 400 mg TID | 28 | 29.34 (8.08) | 11.00 (9.90) | −16.89 (2.04) | −6.75 | 0.0159 | −56.89 (6.92) | −26.61 | 0.0050 |
| (N = 35) | | | | | (−12.23, −1.26) | | | (−45.20, −8.01) | |
| Rilzabrutinib 400 mg BID | 26 | 29.73 (7.11) | 15.61 (13.91) | −14.24 (2.06) | −4.09 | 0.1441 | −48.13 (6.91) | −17.85 | 0.0592 |
| (N = 35) | | | | | (−9.59, 1.40) | | | (−36.39, 0.69) | |
| Rilzabrutinib 400 mg QPM | 30 | 31.11 (7.24) | 22.00 (11.07) | −9.74 (2.00) | 0.40 | 0.8856 | −34.79 (6.80) | −4.51 | 0.6327 |
| (N = 37) | | | | | (−5.08, 5.89) | | | (−22.99, 13.98) | |
| Placebo (N = 36) | 32 | 28.97 (8.39) | 19.32 (13.04) | −10.14 (2.05) | | | −30.28 (6.89) | | |

^{a}Each of the imputed complete data were analyzed by fitting an ANCOVA model with the corresponding baseline value, intervention group and regions as covariates.

TABLE 20
Change and Percent Change from Baseline in UAS7 over Time up to Week 12 (Omalizumab-Naïve Population with
Outlier Removed from Analysis)

| | | | | Change from | Change difference for | | % Change | % Change difference for | |
|---|---|---|---|---|---|---|---|---|---|
| | | | | baseline | rilzabrutinib vs. placebo | | from baseline | rilzabrutinib vs. placebo | |
| | | Baseline | Post-baseline | LS Mean | LS Mean | | LS | LS Mean | p- |
| | n | Mean (SD) | Mean (SD) | (SE)^{a} | (95% CI)ª | p-valueª | Mean (SE)^{a} | (95% CI)^{a} | valueª |
| Week 1 | | | | | | | | | |
| Rilzabrutinib 400 mg TID | 34 | 30.21 (6.36) | 18.29 (9.93) | −11.59 (1.59) | −7.98 (−13.13, −2.83) | 0.0003 | −36.85 (5.65) | −27.32 (−45.67, | 0.0006 |
| (N = 34) | | | | | | | | −8.97) | |
| Rilzabrutinib 400 mg BID | 35 | 29.73 (7.11) | 19.77 (11.32) | −9.85 (1.58) | −6.25 (−11.33, −1.16) | 0.0041 | −34.55 (5.64) | −25.02 (−43.14, | 0.0013 |
| (N = 35) | | | | | | | | −6.90) | |
| Rilzabrutinib 400 mg QPM | 37 | 31.11 (7.24) | 23.81 (8.78) | −7.14 (1.53) | −3.54 (−8.57, 1.49) | 0.0971 | −21.78 (5.44) | −12.25 (−30.18, | 0.1068 |
| (N = 37) | | | | | | | | 5.68) | |
| Placebo (N = 36) | 36 | 28.97 (8.39) | 25.80 (10.44) | −3.60 (1.56) | | | −9.53 (5.57) | | |
| Week 2 | | | | | | | | | |
| Rilzabrutinib 400 mg TID | 34 | 30.21 (6.36) | 16.03 (10.59) | −13.89 (1.58) | −7.96 (−12.18, −3.73) | 0.0002 | −44.94 (5.49) | −27.17 (−41.88, | 0.0003 |
| (N = 34) | | | | | | | | −12.47) | |
| Rilzabrutinib 400 mg BID | 34 | 29.73 (7.11) | 19.59 (10.86) | −10.50 (1.58) | −4.57 (−8.75, −0.39) | 0.0321 | −36.40 (5.49) | −18.63 (−33.17, | 0.0121 |
| (N = 35) | | | | | | | | −4.09) | |
| Rilzabrutinib 400 mg QPM | 37 | 31.11 (7.24) | 23.44 (8.97) | −7.82 (1.52) | −1.89 (−6.02, 2.24) | 0.3700 | −24.47 (5.29) | −6.70 (−21.07, | 0.3612 |
| (N = 37) | | | | | | | | 7.68) | |
| Placebo (N = 36) | 35 | 28.97 (8.39) | 23.49 (10.05) | −5.93 (1.56) | | | −17.77 (5.42) | | |
| Week 3 | | | | | | | | | |
| Rilzabrutinib 400 mg TID | 34 | 30.21 (6.36) | 15.20 (9.97) | −14.55 (1.64) | −7.70 (−12.10, −3.31) | 0.0006 | −47.26 (5.69) | −27.10 (−42.36, | 0.0005 |
| (N = 34) | | | | | | | | −11.83) | |
| Rilzabrutinib 400 mg BID | 35 | 29.73 (7.11) | 17.71 (11.91) | −11.96 (1.63) | −5.12 (−9.46, −0.78) | 0.0207 | −40.21 (5.67) | −20.05 (−35.13, | 0.0092 |
| (N = 35) | | | | | | | | −4.97) | |
| Rilzabrutinib 400 mg QPM | 37 | 31.11 (7.24) | 22.01 (9.00) | −9.28 (1.58) | −2.44 (−6.74, 1.86) | 0.2659 | −29.02 (5.48) | −8.86 (−23.79, | 0.2451 |
| (N = 37) | | | | | | | | 6.08) | |
| Placebo (N = 36) | 35 | 28.97 (8.39) | 22.60 (10.89) | −6.84 (1.62) | | | −20.16 (5.64) | | |
| Week 4 | | | | | | | | | |
| Rilzabrutinib 400 mg TID | 32 | 30.21 (6.36) | 14.65 (9.61) | −14.31 (1.71) | −7.25 (−11.83, −2.67) | 0.0019 | −46.87 (5.93) | −26.13 (−41.99, | 0.0012 |
| (N = 34) | | | | | | | | −10.27) | |
| Rilzabrutinib 400 mg BID | 33 | 29.73 (7.11) | 15.92 (11.71) | −12.88 (1.70) | −5.82 (−10.34, −1.30) | 0.0116 | −43.62 (5.90) | −22.88 (−38.52, | 0.0041 |
| (N = 35) | | | | | | | | −7.25) | |
| Rilzabrutinib 400 mg QPM | 37 | 31.11 (7.24) | 21.92 (8.82) | −9.30 (1.64) | −2.24 (−6.70, 2.22) | 0.3244 | −28.65 (5.66) | −7.91 (−23.33, | 0.3148 |
| (N = 37) | | | | | | | | 7.51) | |
| Placebo (N = 36) | 34 | 28.97 (8.39) | 22.09 (12.20) | −7.06 (1.68) | | | | −20.74 (5.82) | |
| Week 5 | | | | | | | | | |
| Rilzabrutinib 400 mg TID | 33 | 30.21 (6.36) | 13.41 (10.60) | −16.07 (1.72) | −5.61 (−10.23, −0.99) | 0.0172 | −51.99 (5.73) | −19.26 (−34.65, | 0.0142 |
| (N = 34) | | | | | | | | −3.86) | |
| Rilzabrutinib 400 mg BID | 34 | 29.73 (7.11) | 17.22 (13.36) | −12.04 (1.71) | −1.59 (−6.13, 2.96) | 0.4931 | −41.62 (5.69) | −8.89 (−24.04, | 0.2499 |
| (N = 35) | | | | | | | | 6.26) | |
| Rilzabrutinib 400 mg QPM | 36 | 31.11 (7.24) | 20.63 (9.18) | −10.29 (1.65) | 0.16 (−4.35, 4.67) | 0.9433 | −32.16 (5.50) | 0.57 (−14.46, | 0.9411 |
| (N = 37) | | | | | | | | 15.59) | |
| Placebo (N = 36) | 33 | 28.97 (8.39) | 18.31 (9.22) | −10.45 (1.71) | | | −32.73 (5.69) | | |
| Week 6 | | | | | | | | | |
| Rilzabrutinib 400 mg TID | 31 | 30.21 (6.36) | 14.10 (11.23) | −15.69 (1.88) | −6.44 (−11.46, −1.43) | 0.0118 | −50.57 (6.35) | −23.48 (−40.46, | 0.0067 |
| (N = 34) | | | | | | | | −6.49) | |
| Rilzabrutinib 400 mg BID | 34 | 29.73 (7.11) | 17.02 (12.76) | −12.20 (1.86) | −2.96 (−7.92, 2.01) | 0.2434 | −41.32 (6.30) | −14.22 (−31.04, | 0.0973 |
| (N = 35) | | | | | | | | 2.59) | |
| Rilzabrutinib 400 mg QPM | 34 | 31.11 (7.24) | 20.21 (10.42) | −10.66 (1.81) | −1.41 (−6.38, 3.55) | 0.5767 | −33.42 (6.12) | −6.33 (−23.11, | 0.4602 |
| (N = 37) | | | | | | | | 10.46) | |
| Placebo (N = 36) | 32 | 28.97 (8.39) | 19.67 (10.97) | −9.24 (1.87) | | | −27.10 (6.34) | | |
| Week 7 | | | | | | | | | |
| Rilzabrutinib 400 mg TID | 30 | 30.21 (6.36) | 13.54 (9.34) | −16.24 (1.78) | −6.73 (−11.50, −1.96) | 0.0057 | −52.77 (6.02) | −24.12 (−40.27, | 0.0034 |
| (N = 34) | | | | | | | | −7.97) | |
| Rilzabrutinib 400 mg BID | 34 | 29.73 (7.11) | 15.55 (11.97) | −14.01 (1.76) | −4.50 (−9.19, 0.19) | 0.0598 | −47.24 (5.95) | −18.59 (−34.47, | 0.0218 |
| (N = 35) | | | | | | | | −2.70) | |
| Rilzabrutinib 400 mg QPM | 33 | 31.11 (7.24) | 21.11 (10.03) | −10.66 (1.72) | −1.15 (−5.83, 3.52) | 0.6289 | −34.18 (5.81) | −5.53 (−21.38, | 0.4938 |
| (N = 37) | | | | | | | | 10.31) | |
| Placebo (N = 36) | 33 | 28.97 (8.39) | 19.78 (10.84) | −9.51 (1.76) | | | −28.65 (5.95) | | |
| Week 8 | | | | | | | | | |
| Rilzabrutinib 400 mg TID | 28 | 30.21 (6.36) | 13.36 (9.81) | −15.70 (1.90) | −5.68 (−10.80, −0.56) | 0.0298 | −51.40 (6.43) | −20.31 (−37.72, | 0.0222 |
| (N = 34) | | | | | | | | −2.91) | |
| Rilzabrutinib 400 mg BID | 33 | 29.73 (7.11) | 13.98 (13.06) | −15.43 (1.86) | −5.41 (−10.40, −0.43) | 0.0332 | −53.10 (6.28) | −22.01 (−38.88, | 0.0106 |
| (N = 35) | | | | | | | | −5.14) | |
| Rilzabrutinib 400 mg QPM | 33 | 31.11 (7.24) | 21.16 (10.52) | −10.60 (1.81) | −0.58 (−5.54, 4.37) | 0.8181 | −35.12 (6.14) | −4.03 (−20.86, | 0.6385 |
| (N = 37) | | | | | | | | 12.79) | |
| Placebo (N = 36) | 33 | 28.97 (8.39) | 19.31 (11.99) | −10.02 (1.87) | | | −31.09 (6.33) | | |
| Week 9 | | | | | | | | | |
| Rilzabrutinib 400 mg TID | 29 | 30.21 (6.36) | 12.57 (9.72) | −16.22 (1.96) | −7.36 (−12.59, −2.14) | 0.0058 | −52.11 (6.55) | −25.28 (−42.68, | 0.0044 |
| (N = 34) | | | | | | | | −7.89) | |
| Rilzabrutinib 400 mg BID | 29 | 29.73 (7.11) | 14.64 (14.43) | −14.88 (1.92) | −6.02 (−11.15, −0.89) | 0.0215 | −52.05 (6.41) | −25.22 (−42.33, | 0.0039 |
| (N = 35) | | | | | | | | −8.12) | |
| Rilzabrutinib 400 mg QPM | 34 | 31.11 (7.24) | 20.25 (10.25) | −11.11 (1.85) | −2.25 (−7.32, 2.82) | 0.3846 | −35.85 (6.16) | −9.02 (−25.91, | 0.2949 |
| (N = 37) | | | | | | | | 7.86) | |
| Placebo (N = 36) | 32 | 28.97 (8.39) | 20.65 (11.21) | −8.86 (1.91) | | | −26.83 (6.34) | | |
| Week 10 | | | | | | | | | |
| Rilzabrutinib 400 mg TID | 30 | 30.21 (6.36) | 12.61 (9.89) | −16.56 (1.98) | −6.91 (−12.19, −1.64) | 0.0103 | −54.21 (6.69) | −23.84 (−41.69, | 0.0089 |
| (N = 34) | | | | | | | | −5.99) | |
| Rilzabrutinib 400 mg BID | 28 | 29.73 (7.11) | 15.22 (14.36) | −15.06 (1.99) | −5.41 (−10.69, −0.13) | 0.0445 | −51.72 (6.74) | −21.34 (−39.16, | 0.0189 |
| (N = 35) | | | | | | | | −3.52) | |
| Rilzabrutinib 400 mg QPM | 34 | 31.11 (7.24) | 20.27 (10.69) | −11.49 (1.88) | −1.84 (−7.00, 3.33) | 0.4863 | −37.24 (6.37) | −6.86 (−24.33, | 0.4410 |
| (N = 37) | | | | | | | | 10.60) | |
| Placebo (N = 36) | 33 | 28.97 (8.39) | 19.73 (12.24) | −9.65 (1.95) | | | −30.37 (6.58) | | |
| Week 11 | | | | | | | | | |
| Rilzabrutinib 400 mg TID | 31 | 30.21 (6.36) | 13.16 (10.54) | −16.34 (1.95) | −7.03 (−12.30, −1.76) | 0.0090 | −53.14 (6.75) | −24.83 (−43.04, | 0.0076 |
| (N = 34) | | | | | | | | −6.61) | |
| Rilzabrutinib 400 mg BID | 29 | 29.73 (7.11) | 15.06 (13.80) | −14.46 (1.99) | −5.15 (−10.44, 0.15) | 0.0567 | −49.79 (6.87) | −21.48 (−39.77, | 0.0214 |
| (N = 35) | | | | | | | | −3.19) | |
| Rilzabrutinib 400 mg QPM | 33 | 31.11 (7.24) | 21.17 (11.16) | −10.76 (1.90) | −1.45 (−6.67, 3.77) | 0.5864 | −36.00 (6.57) | −7.69 (−25.74, | 0.4037 |
| (N = 37) | | | | | | | | 10.36) | |
| Placebo (N = 36) | 33 | 28.97 (8.39) | 19.89 (12.32) | −9.31 (1.95) | | | −28.31 (6.74) | | |
| Week 12 | | | | | | | | | |
| Rilzabrutinib 400 mg TID | 28 | 30.21 (6.36) | 11.00 (9.90) | −17.50 (2.06) | −7.50 (−13.02, −1.97) | 0.0079 | −57.01 (7.01) | −26.35 (−45.10, | 0.0059 |
| (N = 34) | | | | | | | | −7.61) | |
| Rilzabrutinib 400 mg BID | 26 | 29.73 (7.11) | 15.61 (13.91) | −14.30 (2.06) | −4.29 (−9.74, 1.16) | 0.1232 | −49.10 (7.03) | −18.45 (−37.01, | 0.0515 |
| (N = 35) | | | | | | | | 0.12) | |
| Rilzabrutinib 400 mg QPM | 30 | 31.11 (7.24) | 22.00 (11.07) | −9.93 (1.96) | 0.08 (−5.29, 5.46) | 0.9759 | −34.40 (6.66) | −3.75 (−21.98, | 0.6869 |
| (N = 37) | | | | | | | | 14.48) | |
| Placebo (N = 36) | 32 | 28.97 (8.39) | 19.32 (13.04) | −10.01 (2.01) | | | −30.65 (6.81) | | |

LS: least squares; WOCF: Worst observation carried forward; MI: multiple imputation
^{a}Each of the imputed complete data were analyzed by fitting an ANCOVA model with the corresponding baseline value, intervention group and region as covariates.

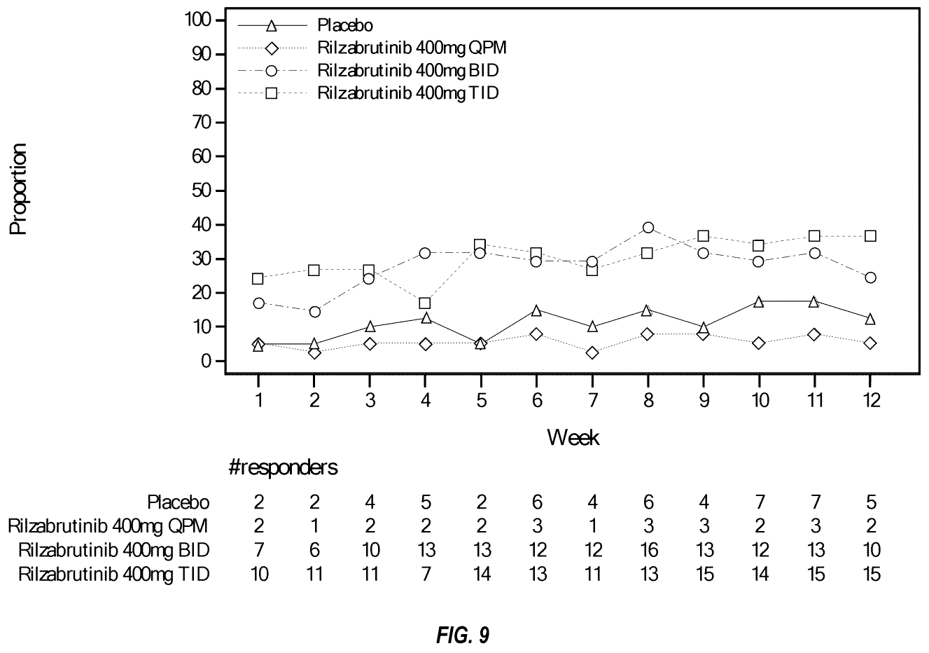

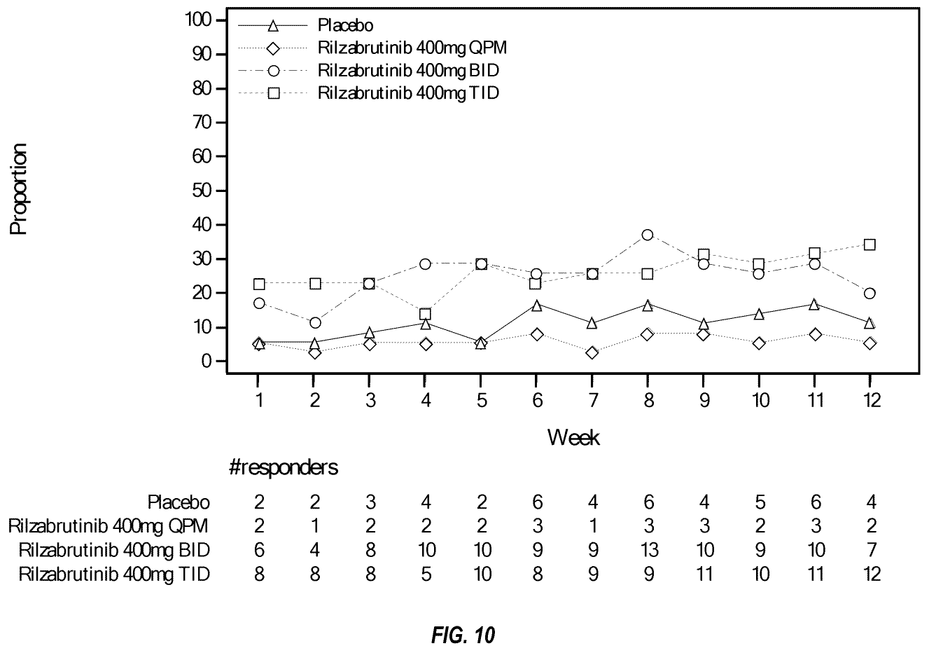

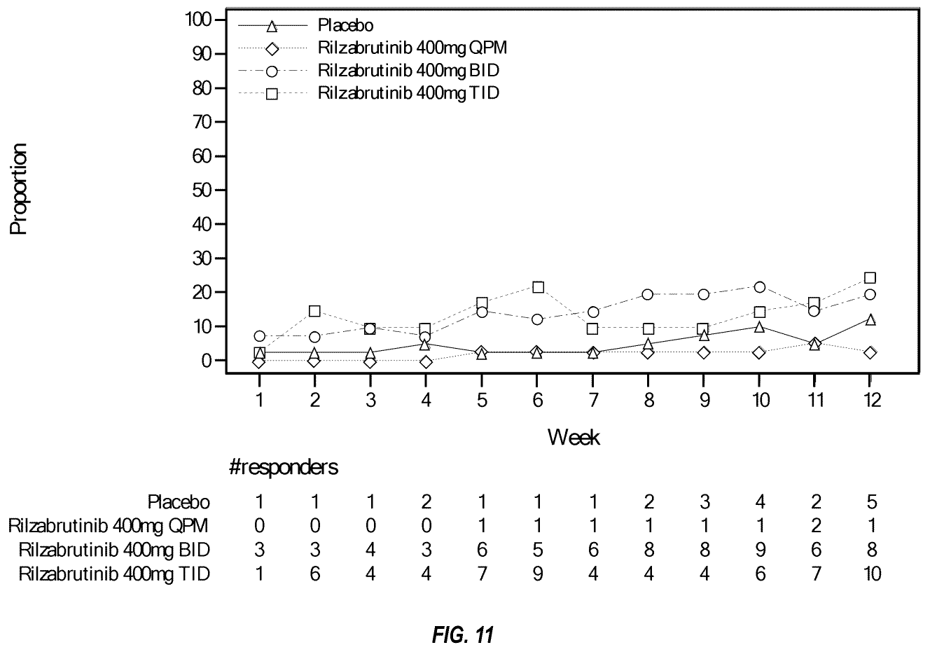

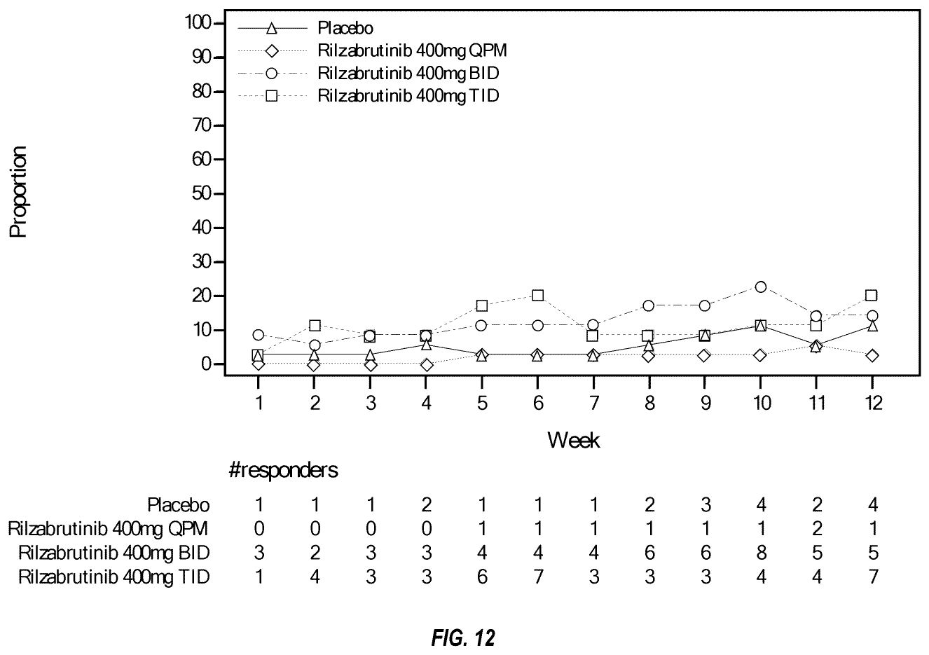

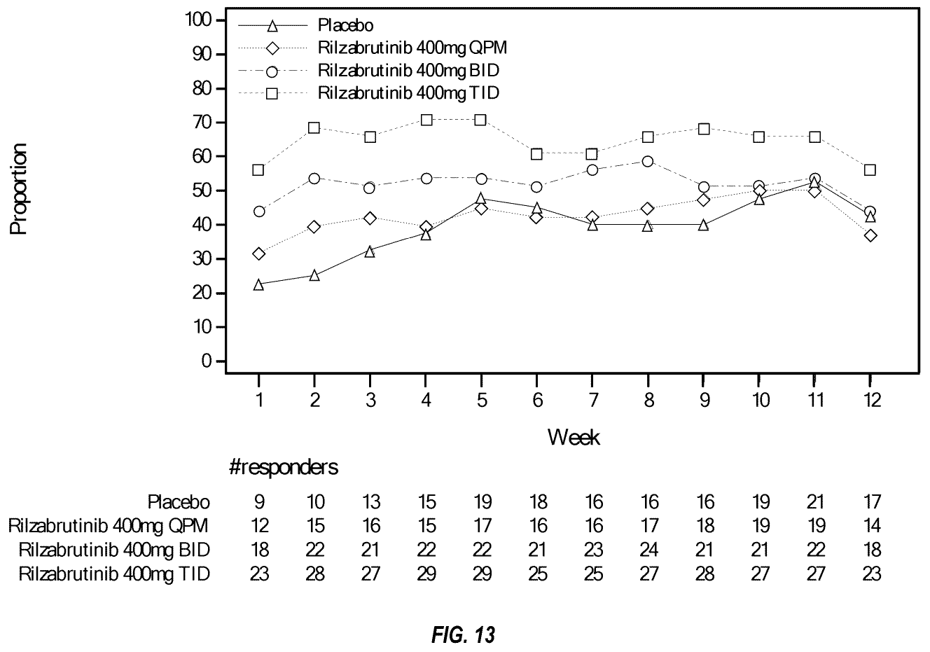

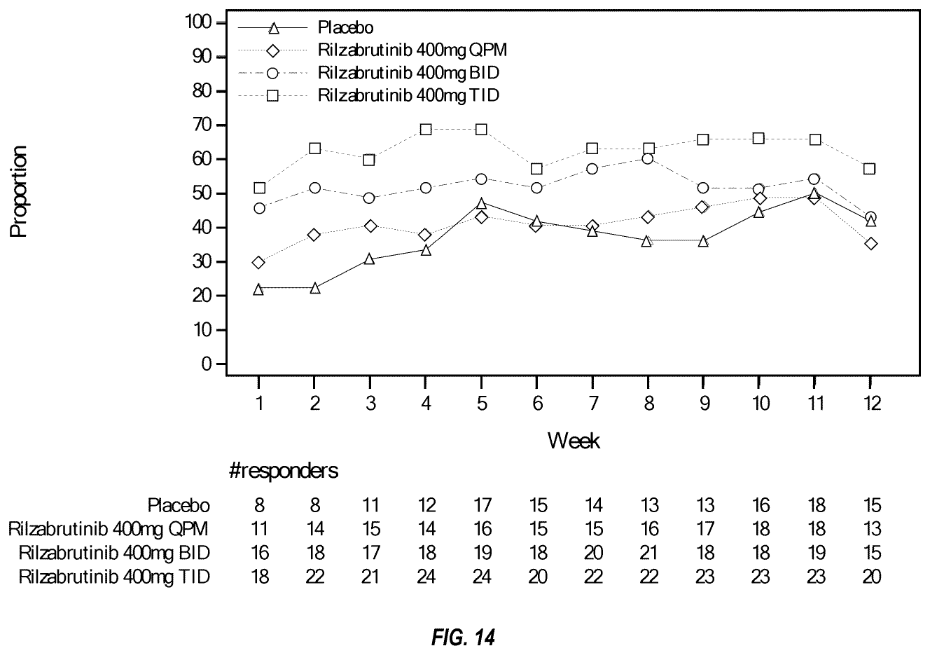

Over the course of the study, a substantial proportion of patients in both the ITT and omalizumab-naïve population achieved a UAS7 score of ≤6 (a non-limiting representative criterion for well-controlled disease), with a smaller population of participants achieving a UAS7 score of 0 (see FIGS. 9-12 and Tables 21-22). At Week 12, 20.0% of omalizumab-naïve participants receiving 400 mg BID rilzabrutinib and 34.3% of omalizumab-naïve participants receiving 400 mg TID rilzabrutinib had a UAS7 of ≤6. Meanwhile, 14.3% of omalizumab-naïve participants in the BID cohort and 20.0% of omalizumab-naïve patients in the TID cohort had a UAS7 of 0. Additionally, a substantial percentage of patients experienced an MCID (a reduction in UAS7 of at least 10 points relative to baseline) that was sustained over the course of the double-blind treatment phase (see FIGS. 13-14 and Table 23).

TABLE 21
Proportion of Participants with UAS7 ≤6 Over Time up to Week 12 (ITT and Omalizumab-Naïve Population)

| ITT Population | | | | | | | Omalizumab-Naïve Population | | | | | |
|---|---|---|---|---|---|---|---|---|---|---|---|---|
| | | | Imputed | RRD (%) | OR | | | | Imputed | RRD (%) | | |
| | | Non- | non- | for | (95% CI) | | | Non- | non- | for | OR (95% | |
| | Re- | re- | re- | rilzabrutinib | for | P-value | Re- | re- | re- | rilzabrutinib | CI) for | P-value |
| | sponder | sponder | sponder | vs. placebo | rilzabrutinib | vs. | sponder | sponder | sponder | vs. placebo | rilzabrutinib | vs. |
| | n (%) | n (%) | n ( %) | (95% CI)ª | vs. placeboª | placebo^{b} | n (%) | n (%) | n (%) | (95% CI)ª | vs. placeboª | placebo^{b} |
| Baseline | | | | | | | | | | | | |
| Rilzabrutinib | 0 | 41 (100) | 0 | | | | 0 | 35 (100) | 0 | | | |
| 400 mg TID | | | | | | | | | | | | |
| (N = 41) | | | | | | | | | | | | |
| Rilzabrutinib | 0 | 41 (100) | 0 | | | | 0 | 35 (100) | 0 | | | |
| 400 mg BID | | | | | | | | | | | | |
| (N = 41) | | | | | | | | | | | | |
| Rilzabrutinib | 0 | 38 (100) | 0 | | | | 0 | 37 (100) | 0 | | | |
| 400 mg QPM | | | | | | | | | | | | |
| (N = 38) | | | | | | | | | | | | |
| Placebo | 0 | 40 (100) | 0 | | | | 0 | 36 (100) | 0 | | | |
| (N = 40) | | | | | | | | | | | | |
| Week 1 | | | | | | | | | | | | |
| Rilzabrutinib | 10 (24.4) | 31 (75.6) | 0 | 17.6 (2.90, | 5.673 (1.111, | 0.0282 | 8 (22.9) | 27 (77.1) | 0 | 15.9 (0.19, | 4.809 (0.890, | 0.0600 |
| 400 mg TID | | | | 32.38) | 28.967) | | | | | 31.52) | 25.993) | |
| (N = 41) | | | | | | | | | | | | |
| Rilzabrutinib | 7 (17.1) | 34 (82.9) | 1 (2.4) | 10.3 (−2.73, | 3.469 (0.658, | 0.1337 | 6 (17.1) | 29 (82.9) | 0 | 9.9 (−4.25, | 3.183 (0.576, | 0.1789 |
| 400 mg BID | | | | 23.37) | 18.287) | | | | | 24.02) | 17.573) | |
| (N = 41) | | | | | | | | | | | | |
| Rilzabrutinib | 2 (5.3) | 36 (94.7) | 0 | 0.4 (−9.14, | 1.099 (0.137, | 0.9314 | 2 (5.4) | 35 (94.6) | 0 | −0.0 | 0.994 (0.125, | 0.9954 |
| 400 mg QPM | | | | 10.01) | 8.792) | | | | | (−10.23, | 7.921) | |
| (N = 38) | | | | | | | | | | 10.17) | | |
| Placebo | 2 (5.0) | 38 (95.0) | 0 | | | | 2 (5.6) | 34 (94.4) | 0 | | | |
| (N = 40) | | | | | | | | | | | | |
| Week 2 | | | | | | | | | | | | |
| Rilzabrutinib | 11 (26.8) | 30 (73.2) | 0 | 19.4 (4.20, | 6.049 (1.232, | 0.0188 | 8 (22.9) | 27 (77.1) | 0 | 15.3 (−0.43, | 4.455 (0.852, | 0.0693 |
| 400 mg TID | | | | 34.60) | 29.703) | | | | | 31.10) | 23.298) | |
| (N = 41) | | | | | | | | | | | | |
| Rilzabrutinib | 6 (14.6) | 35 (85.4) | 2 (4.9) | 7.7 (−4.55, | 2.834 (0.511, | 0.2317 | 4 (11.4) | 31 (88.6) | 1 (2.9) | 4.4 (−7.93, | 1.917 (0.304, | 0.4975 |
| 400 mg BID | | | | 19.88) | 15.715) | | | | | 16.65) | 12.087) | |
| (N = 41) | | | | | | | | | | | | |
| Rilzabrutinib | 1 (2.6) | 37 (97.4) | 0 | −2.3 (−10.71, | 0.517 (0.043, | 0.6072 | 1 (2.7) | 36 (97.3) | 0 | −2.7 | 0.489 (0.042, | 0.5668 |
| 400 mg QPM | | | | 6.12) | 6.233) | | | | | (−11.88, | 5.701) | |
| (N = 38) | | | | | | | | | | 6.43) | | |
| Placebo | 2 (5.0) | 38 (95.0) | 1 (2.5) | | | | 2 (5.6) | 34 (94.4) | 1 (2.8) | | | |
| (N = 40) | | | | | | | | | | | | |
| Week 3 | | | | | | | | | | | | |
| Rilzabrutinib | 11 (26.8) | 30 (73.2) | 0 | 14.9 (−1.46, | 3.055 (0.845, | 0.0887 | 8 (22.9) | 27 (77.1) | 0 | 13.6 (−2.78, | 3.239 (0.735, | 0.1209 |
| 400 mg TID | | | | 31.22) | 11.040) | | | | | 30.07) | 14.279) | |
| (N = 41) | | | | | | | | | | | | |
| Rilzabrutinib | 10 (24.4) | 31 (75.6) | 1 (2.4) | 12.5 (−3.67, | 2.554 (0.730, | 0.1383 | 8 (22.9) | 27 (77.1) | 0 | 13.3 (−3.54, | 2.894 (0.713, | 0.1292 |
| 400 mg BID | | | | 28.69) | 8.938) | | | | | 30.09) | 11.743) | |
| (N = 41) | | | | | | | | | | | | |
| Rilzabrutinib | 2 (5.3) | 36 (94.7) | 0 | −3.9 | 0.553 (0.092, | 0.5202 | 2 (5.4) | 35 (94.6) | 0 | −2.5 | 0.666 (0.102, | 0.6724 |
| 400 mg QPM | | | | (−15.41, | 3.325) | | | | | (−14.01, | 4.335) | |
| (N = 38) | | | | 7.63) | | | | | | 8.99) | | |
| Placebo | 4 (10.0) | 36 (90.0) | 1 (2.5) | | | | 3 (8.3) | 33 (91.7) | 1 (2.8) | | | |
| (N = 40) | | | | | | | | | | | | |
| Week 4 | | | | | | | | | | | | |
| Rilzabrutinib | 7 (17.1) | 34 (82.9) | 3 (7.3) | 2.4 (−13.00, | 1.217 (0.340, | 0.7677 | 5 (14.3) | 30 (85.7) | 3 (8.6) | 1.8 (−13.62, | 1.185 (0.278, | 0.8243 |
| 400 mg TID | | | | 17.75) | 4.362) | | | | | 17.23) | 5.048) | |
| (N = 41) | | | | | | | | | | | | |
| Rilzabrutinib | 13 (31.7) | 28 (68.3) | 4 (9.8) | 16.7 (−0.68, | 2.948 (0.914, | 0.0676 | 10 (28.6) | 25 (71.4) | 2 (5.7) | 15.8 (−2.29, | 2.950 (0.814, | 0.0963 |
| 400 mg BID | | | | 34.05) | 9.514) | | | | | 33.91) | 10.692) | |
| (N = 41) | | | | | | | | | | | | |
| Rilzabrutinib | 2 (5.3) | 36 (94.7) | 1 (2.6) | −6.0 | 0.432 (0.075, | 0.3471 | 2 (5.4) | 35 (94.6) | 1 (2.7) | −5.0 | 0.486 (0.080, | 0.4367 |
| 400 mg QPM | | | | (−18.20, | 2.483) | | | | | (−17.29, | 2.962) | |
| (N = 38) | | | | 6.10) | | | | | | 7.31) | | |
| Placebo | 5 (12.5) | 35 (87.5) | 2 (5.0) | | | | 4 (11.1) | 32 (88.9) | 2 (5.6) | | | |
| (N = 40) | | | | | | | | | | | | |
| Week 5 | | | | | | | | | | | | |
| Rilzabrutinib | 14 (34.1) | 27 (65.9) | 3 (7.3) | 26.0 (9.94, | 8.858 (1.832, | 0.0030 | 10 (28.6) | 25 (71.4) | 2 (5.7) | 19.7 (3.09, | 5.923 (1.166, | 0.0254 |
| 400 mg TID | | | | 42.11) | 42.833) | | | | | 36.22) | 30.099) | |
| (N = 41) | | | | | | | | | | | | |
| Rilzabrutinib | 13 (31.7) | 28 (68.3) | 5 (12.2) | 24.3 (8.61, | 8.203 (1.700, | 0.0037 | 10 (28.6) | 25 (71.4) | 2 (5.7) | 21.1 (4.62, | 6.525 (1.284, | 0.0152 |
| 400 mg BID | | | | 39.95) | 39.569) | | | | | 37.65) | 33.153) | |
| (N = 41) | | | | | | | | | | | | |
| Rilzabrutinib | 2 (5.3) | 36 (94.7) | 2 (5.3) | 0.3 (−9.40, | 1.065 (0.136, | 0.9539 | 2 (5.4) | 35 (94.6) | 2 (5.4) | −0.1 | 0.986 (0.128, | 0.9892 |
| 400 mg QPM | | | | 9.99) | 8.313) | | | | | (−10.46, | 7.566) | |
| (N = 38) | | | | | | | | | | 10.32) | | |
| Placebo | 2 (5.0) | 38 (95.0) | 3 (7.5) | | | | 2 (5.6) | 34 (94.4) | 3 (8.3) | | | |
| (N = 40) | | | | | | | | | | | | |
| Week 6 | | | | | | | | | | | | |
| Rilzabrutinib | 13 (31.7) | 28 (68.3) | 5 (12.2) | 14.2 (−4.24, | 2.273 (0.765, | 0.1405 | 8 (22.9) | 27 (77.1) | 4 (11.4) | 3.7 (−14.85, | 1.274 (0.384, | 0.7003 |
| 400 mg TID | | | | 32.56) | 6.747) | | | | | 22.33) | 4.230) | |
| (N = 41) | | | | | | | | | | | | |
| Rilzabrutinib | 12 (29.3) | 29 (70.7) | 4 (9.8) | 13.6 (−4.03, | 2.329 (0.755, | 0.1450 | 9 (25.7) | 26 (74.3) | 2 (5.7) | 8.8 (−9.86, | 1.743 (0.532, | 0.3678 |
| 400 mg BID | | | | 31.14) | 7.182) | | | | | 27.50) | 5.708) | |
| (N = 41) | | | | | | | | | | | | |
| Rilzabrutinib | 3 (7.9) | 35 (92.1) | 4 (10.5) | −6.3 | 0.540 (0.128, | 0.3899 | 3 (8.1) | 34 (91.9) | 4 (10.8) | −7.8 | 0.481 (0.111, | 0.3189 |
| 400 mg QPM | | | | (−20.67, | 2.276) | | | | | (−22.93, | 2.083) | |
| (N = 38) | | | | 8.03) | | | | | | 7.40) | | |
| Placebo | 6 (15.0) | 34 (85.0) | 6 (15.0) | | | | 6 (16.7) | 30 (83.3) | 5 (13.9) | | | |
| (N = 40) | | | | | | | | | | | | |
| Week 7 | | | | | | | | | | | | |
| Rilzabrutinib | 11 (26.8) | 30 (73.2) | 6 (14.6) | 16.1 (−0.40, | 3.247 (0.898, | 0.0691 | 9 (25.7) | 26 (74.3) | 5 (14.3) | 13.4 (−4.18, | 2.685 (0.704, | 0.1522 |
| 400 mg TID | | | | 32.60) | 11.746) | | | | | 31.05) | 10.244) | |
| (N = 41) | | | | | | | | | | | | |
| Rilzabrutinib | 12 (29.3) | 29 (70.7) | 4 (9.8) | 17.8 (0.98, | 3.425 (0.989, | 0.0465 | 9 (25.7) | 26 (74.3) | 2 (5.7) | 13.6 (−4.13, | 2.622 (0.714, | 0.1456 |
| 400 mg BID | | | | 34.66) | 11.863) | | | | | 31.42) | 9.632) | |
| (N = 41) | | | | | | | | | | | | |
| Rilzabrutinib | 1 (2.6) | 37 (97.4) | 6 (15.8) | −7.3 | 0.278 (0.033, | 0.1981 | 1 (2.7) | 36 (97.3) | 6 (16.2) | −8.3 | 0.249 (0.029, | 0.1674 |
| 400 mg QPM | | | | (−18.32, | 2.339) | | | | | (−20.15, | 2.154) | |
| (N = 38) | | | | 3.75) | | | | | | 3.52) | | |
| Placebo | 4 (10.0) | 36 (90.0) | 5 (12.5) | | | | 4 (11.1) | 32 (88.9) | 4 (11.1) | | | |
| (N = 40) | | | | | | | | | | | | |
| Week 8 | | | | | | | | | | | | |
| Rilzabrutinib | 13 (31.7) | 28 (68.3) | 8 (19.5) | 12.7 (−4.90, | 2.257 (0.723, | 0.1663 | 9 (25.7) | 26 (74.3) | 7 (20.0) | 6.3 (−12.17, | 1.513 (0.448, | 0.5163 |
| 400 mg TID | | | | 30.33) | 7.043) | | | | | 24.68) | 5.104) | |
| (N = 41) | | | | | | | | | | | | |
| Rilzabrutinib | 16 (39.0) | 25 (61.0) | 5 (12.2) | 21.8 (3.07, | 3.297 (1.126, | 0.0279 | 13 (37.1) | 22 (62.9) | 3 (8.6) | 18.5 (−1.55, | 2.745 (0.891, | 0.0799 |
| 400 mg BID | | | | 40.46) | 9.656) | | | | | 38.58) | 8.452) | |
| (N = 41) | | | | | | | | | | | | |
| Rilzabrutinib | 3 (7.9) | 35 (92.1) | 7 (18.4) | −7.0 | 0.502 (0.118, | 0.3464 | 3 (8.1) | 34 (91.9) | 7 (18.9) | −8.4 | 0.454 (0.105, | 0.2869 |
| 400 mg QPM | | | | (−21.26, | 2.138) | | | | | (−23.55, | 1.963) | |
| (N = 38) | | | | 7.28) | | | | | | 6.78) | | |
| Placebo | 6 (15.0) | 34 (85.0) | 5 (12.5) | | | | 6 (16.7) | 30 (83.3) | 4 (11.1) | | | |
| (N = 40) | | | | | | | | | | | | |
| Week 9 | | | | | | | | | | | | |
| Rilzabrutinib | 15 (36.6) | 26 (63.4) | 7 (17.1) | 22.0 (4.85, | 4.680 (1.311, | 0.0151 | 11 (31.4) | 24 (68.6) | 6 (17.1) | 16.7 (−1.21, | 3.358 (0.885, | 0.0781 |
| 400 mg TID | | | | 39.06) | 16.709) | | | | | 34.64) | 12.743) | |
| (N = 41) | | | | | | | | | | | | |
| Rilzabrutinib | 13 (31.7) | 28 (68.3) | 9 (22.0) | 19.4 (2.08, | 3.540 (1.061, | 0.0309 | 10 (28.6) | 25 (71.4) | 7 (20.0) | 15.6 (−2.80, | 2.797 (0.796, | 0.0998 |
| 400 mg BID | | | | 36.75) | 11.804) | | | | | 34.02) | 9.830) | |
| (N = 41) | | | | | | | | | | | | |
| Rilzabrutinib | 3 (7.9) | 35 (92.1) | 6 (15.8) | −2.2 (−15.39, | 0.773 (0.169, | 0.7375 | 3 (8.1) | 34 (91.9) | 6 (16.2) | −3.0 | 0.715 (0.154, | 0.6671 |
| 400 mg QPM | | | | 10.92) | 3.540) | | | | | (−16.97, | 3.319) | |
| (N = 38) | | | | | | | | | | 10.87) | | |
| Placebo | 4 (10.0) | 36 (90.0) | 6 (15.0) | | | | 4 (11.1) | 32 (88.9) | 5 (13.9) | | | |
| (N = 40) | | | | | | | | | | | | |
| Week 10 | | | | | | | | | | | | |
| Rilzabrutinib | 14 (34.1) | 27 (65.9) | 7 (17.1) | 12.9 (−5.77, | 2.074 (0.709, | 0.1797 | 10 (28.6) | 25 (71.4) | 5 (14.3) | 12.1 (−6.36, | 2.241 (0.642, | 0.2111 |
| 400 mg TID | | | | 31.54) | 6.063) | | | | | 30.46) | 7.824) | |
| (N = 41) | | | | | | | | | | | | |
| Rilzabrutinib | 12 (29.3) | 29 (70.7) | 10 (24.4) | 8.6 (−9.13, | 1.699 (0.567, | 0.3472 | 9 (25.7) | 26 (74.3) | 8 (22.9) | 9.9 (−8.41, | 1.923 (0.566, | 0.2961 |
| 400 mg BID | | | | 26.41) | 5.093) | | | | | 28.23) | 6.530) | |
| (N = 41) | | | | | | | | | | | | |
| Rilzabrutinib | 2 (5.3) | 36 (94.7) | 6 (15.8) | −11.9 | 0.279 (0.055, | 0.1090 | 2 (5.4) | 35 (94.6) | 6 (16.2) | −8.6 | 0.357 (0.066, | 0.2263 |
| 400 mg QPM | | | | (−25.79, | 1.407) | | | | | (−22.21, | 1.940) | |
| (N = 38) | | | | 2.04) | | | | | | 5.00) | | |
| Placebo | 7 (17.5) | 33 (82.5) | 5 (12.5) | | | | 5 (13.9) | 31 (86.1) | 4 (11.1) | | | |
| (N = 40) | | | | | | | | | | | | |
| Week 11 | | | | | | | | | | | | |
| Rilzabrutinib | 15 (36.6) | 26 (63.4) | 6 (14.6) | 15.0 (−3.73, | 2.322 (0.795, | 0.1220 | 11 (31.4) | 24 (68.6) | 4 (11.4) | 11.2 (−8.12, | 1.971 (0.605, | 0.2662 |
| 400 mg TID | | | | 33.81) | 6.782) | | | | | 30.44) | 6.420) | |
| (N = 41) | | | | | | | | | | | | |
| Rilzabrutinib | 13 (31.7) | 28 (68.3) | 9 (22.0) | 11.8 (−6.40, | 1.991 (0.676, | 0.2172 | 10 (28.6) | 25 (71.4) | 7 (20.0) | 10.3 (−8.89, | 1.853 (0.578, | 0.3072 |
| 400 mg BID | | | | 30.04) | 5.862) | | | | | 29.45) | 5.939) | |
| (N = 41) | | | | | | | | | | | | |
| Rilzabrutinib | 3 (7.9) | 35 (92.1) | 7 (18.4) | −9.4 | 0.418 (0.101, | 0.2244 | 3 (8.1) | 34 (91.9) | 7 (18.9) | −8.5 | 0.456 (0.107, | 0.2834 |
| 400 mg QPM | | | | (−24.19, | 1.739) | | | | | (−23.75, | 1.946) | |
| (N = 38) | | | | 5.35) | | | | | | 6.80) | | |
| Placebo | 7 (17.5) | 33 (82.5) | 5 (12.5) | | | | 6 (16.7) | 30 (83.3) | 4 (11.1) | | | |
| (N = 40) | | | | | | | | | | | | |
| Week 12 | | | | | | | | | | | | |
| Rilzabrutinib | 15 (36.6) | 26 (63.4) | 11 (26.8) | 20.9 (3.11, | 3.721 (1.141, | 0.0271 | 12 (34.3) | 23 (65.7) | 8 (22.9) | 20.3 (1.86, | 3.980 (1.064, | 0.0394 |
| 400 mg TID | | | | 38.70) | 12.137) | | | | | 38.75) | 14.887) | |
| (N = 41) | | | | | | | | | | | | |
| Rilzabrutinib | 10 (24.4) | 31 (75.6) | 14 (34.1) | 10.0 (−6.37, | 2.067 (0.616, | 0.2480 | 7 (20.0) | 28 (80.0) | 11 (31.4) | 7.9 (−8.61, | 1.904 (0.488, | 0.3661 |
| 400 mg BID | | | | 26.39) | 6.930) | | | | | 24.44) | 7.430) | |
| (N = 41) | | | | | | | | | | | | |
| Rilzabrutinib | 2 (5.3) | 36 (94.7) | 10 (26.3) | −7.6 | 0.378 (0.069, | 0.2583 | 2 (5.4) | 35 (94.6) | 10 (27.0) | −6.1 | 0.438 (0.076, | 0.3551 |
| 400 mg QPM | | | | (−20.17, | 2.072) | | | | | (−18.87, | 2.527) | |
| (N = 38) | | | | 5.06) | | | | | | 6.62) | | |
| Placebo | 5 (12.5) | 35 (87.5) | 7 (17.5) | | | | 4 (11.1) | 32 (88.9) | 5 (13.9) | | | |
| (N = 40) | | | | | | | | | | | | |

ªOR: odds ratio; RRD: response rate difference; derived from the Mantel-Haenszel estimator
^{b}CMH test was performed on the association between the responder status and intervention group, stratified by region.

TABLE 22
Proportion of Participants with UAS7 = 0 over Time up to Week 12 (ITT Population and Omalizumab-Naïve
Participants)

| | ITT Population | | | | | | Omalizumab-Naïve Population | | | | | |
|---|---|---|---|---|---|---|---|---|---|---|---|---|
| | | | Imputed | RRD (%) for | OR (95% | | | Non- | Imputed | RRD (%) for | OR (95% | |
| | Re- | Non- | non- | rilzabrutinib | CI) for | P-value | Re- | re- | non- | rilzabrutinib | CI) for | P-value |
| | sponder | responder | responder | vs. placebo | rilzabrutinib | vs. | sponder | sponder | responder | vs. placebo | rilzabrutinib | vs. |
| | n (%) | n (%) | n (%) | (95% CI)^{a} | vs. placeboª | placebo^{b} | n (%) | n (%) | n ( %) | (95% CI)^{a} | vs. placeboª | placebo^{b} |
| Baseline | | | | | | | | | | | | |
| Rilzabrutinib | 0 | 41 (100) | 0 | | | | 0 | 35 (100) | 0 | | | |
| 400 mg TID | | | | | | | | | | | | |
| (N = 41) | | | | | | | | | | | | |
| Rilzabrutinib | 0 | 41 (100) | 0 | | | | 0 | 35 (100) | 0 | | | |
| 400 mg BID | | | | | | | | | | | | |
| (N = 41) | | | | | | | | | | | | |
| Rilzabrutinib | 0 | 38 (100) | 0 | | | | 0 | 37 (100) | 0 | | | |
| 400 mg QPM | | | | | | | | | | | | |
| (N = 38) | | | | | | | | | | | | |
| Placebo (N = 40) | 0 | 40 (100) | 0 | | | | 0 | 36 (100) | 0 | | | |
| Week 1 | | | | | | | | | | | | |
| Rilzabrutinib | 1 (2.4) | 40 (97.6) | 0 | 0.2 (−6.20, | 1.084 (0.050, | 0.9622 | 1 (2.9) | 34 (97.1) | 0 | 0.2 (−6.96, | 1.111 (0.050, | 0.9515 |
| 400 mg TID | | | | 6.54) | 23.369) | | | | | 7.45) | 24.475) | |
| (N = 41) | | | | | | | | | | | | |
| Rilzabrutinib | 3 (7.3) | 38 (92.7) | 1 (2.4) | 4.7 (−4.53, | 3.132 (0.292, | 0.3406 | 3 (8.6) | 32 (91.4) | 0 | 5.6 (−5.06, | 3.242 (0.306, | 0.3217 |
| 400 mg BID | | | | 13.93) | 33.570) | | | | | 16.16) | 34.369) | |
| (N = 41) | | | | | | | | | | | | |
| Rilzabrutinib | 0 | 38 (100) | 0 | −2.6 (−7.54, | 0.000 (NE, | 0.3173 | 0 | 37 (100) | 0 | −2.9 (−8.32, | 0.000 (NE, | 0.2994 |
| 400 mg QPM | | | | 2.36) | NE) | | | | | 2.60) | NE) | |
| (N = 38) | | | | | | | | | | | | |
| Placebo (N = 40) | 1 (2.5) | 39 (97.5) | 0 | | | | 1 (2.8) | 35 (97.2) | 0 | | | |
| Week 2 | | | | | | | | | | | | |
| Rilzabrutinib | 6 (14.6) | 35 (85.4) | 0 | 11.0 (−0.61, | 6.522 (0.677, | 0.0852 | 4 (11.4) | 31 (88.6) | 0 | 8.3 (−3.37, | 4.736 (0.439, | 0.1936 |
| 400 mg TID | | | | 22.60) | 62.812) | | | | | 19.87) | 51.037) | |
| (N = 41) | | | | | | | | | | | | |
| Rilzabrutinib | 3 (7.3) | 38 (92.7) | 2 (4.9) | 4.2 (−4.81, | 2.893 (0.262, | 0.3887 | 2 (5.7) | 33 (94.3) | 1 (2.9) | 2.5 (−6.51, | 2.024 (0.158, | 0.5993 |
| 400 mg BID | | | | 13.16) | 31.979) | | | | | 11.58) | 25.945) | |
| (N = 41) | | | | | | | | | | | | |
| Rilzabrutinib | 0 | 38 (100) | 0 | −2.6 (−7.54, | 0.000 (NE, | 0.3173 | 0 | 37 (100) | 0 | −2.9 (−8.32, | 0.000 (NE, | 0.2994 |
| 400 mg QPM | | | | 2.36) | NE) | | | | | 2.60) | NE) | |
| (N = 38) | | | | | | | | | | | | |
| Placebo (N = 40) | 1 (2.5) | 39 (97.5) | 1 (2.5) | | | | 1 (2.8) | 35 (97.2) | 1 (2.8) | | | |
| Week 3 | | | | | | | | | | | | |
| Rilzabrutinib | 4 (9.8) | 37 (90.2) | 0 | 6.9 (−3.14, | 4.480 (0.413, | 0.2129 | 3 (8.6) | 32 (91.4) | 0 | 5.8 (−4.73, | 3.625 (0.312, | 0.3109 |
| 400 mg TID | | | | 17.00) | 48.581) | | | | | 16.32) | 42.079) | |
| (N = 41) | | | | | | | | | | | | |
| Rilzabrutinib | 4 (9.8) | 37 (90.2) | 1 (2.4) | 6.3 (−3.66, | 3.857 (0.387, | 0.2374 | 3 (8.6) | 32 (91.4) | 0 | 5.0 (−5.34, | 3.036 (0.283, | 0.3569 |
| 400 mg BID | | | | 16.26) | 38.430) | | | | | 15.43) | 32.571) | |
| (N = 41) | | | | | | | | | | | | |
| Rilzabrutinib | 0 | 38 (100) | 0 | −2.6 (−7.54, | 0.000 (NE, | 0.3173 | 0 | 37 (100) | 0 | −2.9 (−8.32, | 0.000 (NE, | 0.2994 |
| 400 mg QPM | | | | 2.36) | NE) | | | | | 2.60) | NE) | |
| (N = 38) | | | | | | | | | | | | |
| Placebo (N = 40) | 1 (2.5) | 39 (97.5) | 1 (2.5) | | | | 1 (2.8) | 35 (97.2) | 1 (2.8) | | | |
| Week 4 | | | | | | | | | | | | |
| Rilzabrutinib | 4 (9.8) | 37 (90.2) | 3 (7.3) | 4.0 (−7.30, | 1.867 (0.308, | 0.5079 | 3 (8.6) | 32 (91.4) | 3 (8.6) | 2.5 (−9.62, | 1.460 (0.224, | 0.6991 |
| 400 mg TID | | | | 15.30) | 11.311) | | | | | 14.53) | 9.499) | |
| (N = 41) | | | | | | | | | | | | |
| Rilzabrutinib | 3 (7.3) | 38 (92.7) | 4 (9.8) | 1.3 (−8.78, | 1.278 (0.186, | 0.8091 | 3 (8.6) | 32 (91.4) | 2 (5.7) | 1.9 (−9.53, | 1.372 (0.197, | 0.7574 |
| 400 mg BID | | | | 11.36) | 8.766) | | | | | 13.23) | 9.566) | |
| (N = 41) | | | | | | | | | | | | |
| Rilzabrutinib | 0 | 38 (100) | 1 (2.6) | −4.8 (−11.39, | 0.000 (NE, | 0.1917 | 0 | 37 (100) | 1 (2.7) | −5.3 (−12.71, | 0.000 (NE, | 0.1696 |
| 400 mg QPM | | | | 1.89) | NE) | | | | | 2.03) | NE) | |
| (N = 38) | | | | | | | | | | | | |
| Placebo (N = 40) | 2 (5.0) | 38 (95.0) | 2 (5.0) | | | | 2 (5.6) | 34 (94.4) | 2 (5.6) | | | |
| Week 5 | | | | | | | | | | | | |
| Rilzabrutinib | 7 (17.1) | 34 (82.9) | 3 (7.3) | 13.2 (0.86, | 7.606 (0.820, | 0.0523 | 6 (17.1) | 29 (82.9) | 2 (5.7) | 13.2 (−0.23, | 6.971 (0.745, | 0.0692 |
| 400 mg TID | | | | 25.45) | 70.599) | | | | | 26.61) | 65.267) | |
| (N = 41) | | | | | | | | | | | | |
| Rilzabrutinib | 6 (14.6) | 35 (85.4) | 5 (12.2) | 11.1 (−0.64, | 6.024 (0.684, | 0.0771 | 4 (11.4) | 31 (88.6) | 2 (5.7) | 8.1 (−3.64, | 4.254 (0.439, | 0.1914 |
| 400 mg BID | | | | 22.79) | 53.045) | | | | | 19.77) | 41.171) | |
| (N = 41) | | | | | | | | | | | | |
| Rilzabrutinib | 1 (2.6) | 37 (97.4) | 2 (5.3) | 0.0 (−6.93, | 1.000 (0.056, | 1.0000 | 1 (2.7) | 36 (97.3) | 2 (5.4) | −0.2 (−7.56, | 0.923 (0.052, | 0.9574 |
| 400 mg QPM | | | | 6.93) | 17.751) | | | | | 7.15) | 16.456) | |
| (N = 38) | | | | | | | | | | | | |
| Placebo (N = 40) | 1 (2.5) | 39 (97.5) | 3 (7.5) | | | | 1 (2.8) | 35 (97.2) | 3 (8.3) | | | |
| Week 6 | | | | | | | | | | | | |
| Rilzabrutinib | 9 (22.0) | 32 (78.0) | 5 (12.2) | 17.2 (3.80, | 9.649 (1.140, | 0.0183 | 7 (20.0) | 28 (80.0) | 4 (11.4) | 15.6 (1.47, | 8.082 (0.897, | 0.0415 |
| 400 mg TID | | | | 30.65) | 81.651) | | | | | 29.81) | 72.821) | |
| (N = 41) | | | | | | | | | | | | |
| Rilzabrutinib | 5 (12.2) | 36 (87.8) | 4 (9.8) | 8.4 (−2.38, | 4.821 (0.522, | 0.1426 | 4 (11.4) | 31 (88.6) | 2 (5.7) | 7.6 (−3.96, | 4.048 (0.423, | 0.2081 |
| 400 mg BID | | | | 19.23) | 44.499) | | | | | 19.07) | 38.762) | |
| (N = 41) | | | | | | | | | | | | |
| Rilzabrutinib | 1 (2.6) | 37 (97.4) | 4 (10.5) | 0.0 (−6.93, | 1.000 (0.056, | 1.0000 | 1 (2.7) | 36 (97.3) | 4 (10.8) | −0.2 (−7.56, | 0.923 (0.052, | 0.9574 |
| 400 mg QPM | | | | 6.93) | 17.751) | | | | | 7.15) | 16.456) | |
| (N = 38) | | | | | | | | | | | | |
| Placebo (N = 40) | 1 (2.5) | 39 (97.5) | 6 (15.0) | | | | 1 (2.8) | 35 (97.2) | 5 (13.9) | | | |
| Week 7 | | | | | | | | | | | | |
| Rilzabrutinib | 4 (9.8) | 37 (90.2) | 6 (14.6) | 6.9 (−3.14, | 4.480 (0.413, | 0.2129 | 3 (8.6) | 32 (91.4) | 5 (14.3) | 5.8 (−4.73, | 3.625 (0.312, | 0.3109 |
| 400 mg TID | | | | 17.00) | 48.581) | | | | | 16.32) | 42.079) | |
| (N = 41) | | | | | | | | | | | | |
| Rilzabrutinib | 6 (14.6) | 35 (85.4) | 4 (9.8) | 11.1 (−0.64, | 6.024 (0.684, | 0.0771 | 4 (11.4) | 31 (88.6) | 2 (5.7) | 8.1 (−3.64, | 4.254 (0.439, | 0.1914 |
| 400 mg BID | | | | 22.79) | 53.045) | | | | | 19.77) | 41.171) | |
| (N = 41) | | | | | | | | | | | | |
| Rilzabrutinib | 1 (2.6) | 37 (97.4) | 6 (15.8) | 0.4 (−7.05, | 1.167 (0.081, | 0.9055 | 1 (2.7) | 36 (97.3) | 6 (16.2) | 0.2 (−7.72, | 1.061 (0.073, | 0.9644 |
| 400 mg QPM | | | | 7.92) | 16.709) | | | | | 8.07) | 15.484) | |
| (N = 38) | | | | | | | | | | | | |
| Placebo (N = 40) | 1 (2.5) | 39 (97.5) | 5 (12.5) | | | | 1 (2.8) | 35 (97.2) | 4 (11.1) | | | |
| Week 8 | | | | | | | | | | | | |
| Rilzabrutinib | 4 (9.8) | 37 (90.2) | 8 (19.5) | 3.2 (−7.77, | 1.697 (0.273, | 0.5836 | 3 (8.6) | 32 (91.4) | 7 (20.0) | 1.9 (−9.95, | 1.351 (0.204, | 0.7599 |
| 400 mg TID | | | | 14.20) | 10.531) | | | | | 13.68) | 8.952) | |
| (N = 41) | | | | | | | | | | | | |
| Rilzabrutinib | 8 (19.5) | 33 (80.5) | 5 (12.2) | 13.4 (−0.62, | 4.083 (0.825, | 0.0725 | 6 (17.1) | 29 (82.9) | 3 (8.6) | 10.7 (−3.97, | 3.154 (0.607, | 0.1632 |
| 400 mg BID | | | | 27.32) | 20.197) | | | | | 25.45) | 16.397) | |
| (N = 41) | | | | | | | | | | | | |
| Rilzabrutinib | 1 (2.6) | 37 (97.4) | 7 (18.4) | −2.2 (−10.44, | 0.527 (0.042, | 0.6245 | 1 (2.7) | 36 (97.3) | 7 (18.9) | −2.7 (−11.63, | 0.477 (0.039, | 0.5680 |
| 400 mg QPM | | | | 6.12) | 6.557) | | | | | 6.26) | 5.895) | |
| (N = 38) | | | | | | | | | | | | |
| Placebo (N = 40) | 2 (5.0) | 38 (95.0) | 5 (12.5) | | | | 2 (5.6) | 34 (94.4) | 4 (11.1) | | | |
| Week 9 | | | | | | | | | | | | |
| Rilzabrutinib | 4 (9.8) | 37 (90.2) | 7 (17.1) | 0.8 (−11.22, | 1.117 (0.220, | 0.8981 | 3 (8.6) | 32 (91.4) | 6 (17.1) | −0.9 (−13.88, | 0.893 (0.161, | 0.8989 |
| 400 mg TID | | | | 12.86) | 5.670) | | | | | 12.16) | 4.971) | |
| (N = 41) | | | | | | | | | | | | |
| Rilzabrutinib | 8 (19.5) | 33 (80.5) | 9 (22.0) | 11.3 (−3.19, | 2.914 (0.694, | 0.1426 | 6 (17.1) | 29 (82.9) | 7 (20.0) | 8.4 (−6.86, | 2.249 (0.502, | 0.2940 |
| 400 mg BID | | | | 25.78) | 12.243) | | | | | 23.71) | 10.063) | |
| (N = 41) | | | | | | | | | | | | |
| Rilzabrutinib | 1 (2.6) | 37 (97.4) | 6 (15.8) | −4.9 (−14.54, | 0.330 (0.032, | 0.3393 | 1 (2.7) | 36 (97.3) | 6 (16.2) | −5.6 (−16.00, | 0.305 (0.029, | 0.3052 |
| 400 mg QPM | | | | 4.76) | 3.409) | | | | | 4.83) | 3.168) | |
| (N = 38) | | | | | | | | | | | | |
| Placebo (N = 40) | 3 (7.5) | 37 (92.5) | 6 (15.0) | | | | 3 (8.3) | 33 (91.7) | 5 (13.9) | | | |
| Week 10 | | | | | | | | | | | | |
| Rilzabrutinib | 6 (14.6) | 35 (85.4) | 7 (17.1) | 1.7 (−11.54, | 1.210 (0.276, | 0.8084 | 4 (11.4) | 31 (88.6) | 5 (14.3) | −1.7 (−15.55, | 0.819 (0.163, | 0.8173 |
| 400 mg TID | | | | 14.96) | 5.304) | | | | | 12.11) | 4.120) | |
| (N = 41) | | | | | | | | | | | | |
| Rilzabrutinib | 9 (22.0) | 32 (78.0) | 10 (24.4) | 10.9 (−4.98, | 2.322 (0.651, | 0.1903 | 8 (22.9) | 27 (77.1) | 8 (22.9) | 10.6 (−6.91, | 2.159 (0.588, | 0.2438 |
| 400 mg BID | | | | 26.81) | 8.283) | | | | | 28.09) | 7.919) | |
| (N = 41) | | | | | | | | | | | | |
| Rilzabrutinib | 1 (2.6) | 37 (97.4) | 6 (15.8) | −7.9 (−18.71, | 0.235 (0.026, | 0.1701 | 1 (2.7) | 36 (97.3) | 6 (16.2) | −8.7 (−20.44, | 0.219 (0.024, | 0.1517 |
| 400 mg QPM | | | | 3.01) | 2.134) | | | | | 2.97) | 2.012) | |
| (N = 38) | | | | | | | | | | | | |
| Placebo (N = 40) | 4 (10.0) | 36 (90.0) | 5 (12.5) | | | | 4 (11.1) | 32 (88.9) | 4 (11.1) | | | |
| Week 11 | | | | | | | | | | | | |
| Rilzabrutinib | 7 (17.1) | 34 (82.9) | 6 (14.6) | 10.4 (−2.82, | 3.449 (0.661, | 0.1330 | 4 (11.4) | 31 (88.6) | 4 (11.4) | 4.3 (−8.37, | 1.846 (0.304, | 0.5161 |
| 400 mg TID | | | | 23.58) | 18.002) | | | | | 17.01) | 11.223) | |
| (N = 41) | | | | | | | | | | | | |
| Rilzabrutinib | 6 (14.6) | 35 (85.4) | 9 (22.0) | 8.2 (−4.30, | 2.892 (0.532, | 0.2142 | 5 (14.3) | 30 (85.7) | 7 (20.0) | 7.4 (−6.07, | 2.551 (0.444, | 0.2939 |
| 400 mg BID | | | | 20.69) | 15.710) | | | | | 20.83) | 14.640) | |
| (N = 41) | | | | | | | | | | | | |
| Rilzabrutinib | 2 (5.3) | 36 (94.7) | 7 (18.4) | 0.3 (−9.40, | 1.065 (0.136, | 0.9539 | 2 (5.4) | 35 (94.6) | 7 (18.9) | −0.1 (−10.46, | 0.986 (0.128, | 0.9892 |
| 400 mg QPM | | | | 9.99) | 8.313) | | | | | 10.32) | 7.566) | |
| (N = 38) | | | | | | | | | | | | |
| Placebo (N = 40) | 2 (5.0) | 38 (95.0) | 5 (12.5) | | | | 2 (5.6) | 34 (94.4) | 4 (11.1) | | | |
| Week 12 | | | | | | | | | | | | |
| Rilzabrutinib | 10 (24.4) | 31 (75.6) | 11 (26.8) | 9.4 (−7.01, | 1.995 (0.587, | 0.2755 | 7 (20.0) | 28 (80.0) | 8 (22.9) | 6.9 (−9.40, | 1.800 (0.444, | 0.4283 |
| 400 mg TID | | | | 25.85) | 6.780) | | | | | 23.14) | 7.300) | |
| (N = 41) | | | | | | | | | | | | |
| Rilzabrutinib | 8 (19.5) | 33 (80.5) | 14 (34.1) | 5.4 (−10.49, | 1.511 (0.443, | 0.5151 | 5 (14.3) | 30 (85.7) | 11 (31.4) | 2.6 (−13.10, | 1.258 (0.308, | 0.7536 |
| 400 mg BID | | | | 21.25) | 5.159) | | | | | 18.22) | 5.128) | |
| (N = 41) | | | | | | | | | | | | |
| Rilzabrutinib | 1 (2.6) | 37 (97.4) | 10 (26.3) | −10.0 (−21.65, | 0.194 (0.022, | 0.1076 | 1 (2.7) | 36 (97.3) | 10 (27.0) | −8.7 (−20.44, | 0.219 (0.024, | 0.1517 |
| 400 mg QPM | | | | 1.62) | 1.698) | | | | | 2.97) | 2.012) | |
| (N = 38) | | | | | | | | | | | | |
| Placebo (N = 40) | 5 (12.5) | 35 (87.5) | 7 (17.5) | | | | 4 (11.1) | 32 (88.9) | 5 (13.9) | | | |

^{a}OR: odds ratio; RRD: response rate difference; derived from the Mantel-Haenszel estimator
^{b}CMH test was performed on the association between the responder status and intervention group, stratified by region.

TABLE 23
Proportion of participants with UAS7 reduction from baseline of 10 points or more over time up to Week 12-ITT
population

| | ITT Population | | | | | | Omalizumab-Naive Participants | | | | | |
|---|---|---|---|---|---|---|---|---|---|---|---|---|
| | | | | RRD (%) | | | | | | RRD (%) | | |
| | | | Imputed | for | OR (95% | | | Non- | Imputed | for | OR (95% | |
| | Re- | Non- | non- | rilzabrutinib | CI) for | P-value | Re- | re- | non- | vs. placebo | CI) for | P-value |
| | sponder | responder | responder | vs. placebo | rilzabrutinib | vs. | sponder | sponder | responderrilzabrutinibrilzabrutinib | | | vs. |
| | n (%) | n (%) | n (%) | (95% CI)ª | vs. placeboª | placebo^{b} | n (%) | n (%) | n (%) | (95% CI)ª | vs. placeboª | placebo^{b} |
| Week 1 | | | | | | | | | | | | |
| Rilzabrutinib 400 mg TID | 23 (56.1) | 18 (43.9) | 0 | 32.5 (12.09, | 4.196 (1.583, | 0.0037 | 18 (51.4) | 17 (48.6) | 0 | 28.5 (6.70, | 3.580 (1.268, | 0.0162 |
| (N = 41) | | | | 52.85) | 11.120) | | | | | 50.35) | 10.109) | |
| Rilzabrutinib 400 mg BID | 18 (43.9) | 23 (56.1) | 1 (2.4) | 20.0 (0.12, | 2.607 (0.975, | 0.0560 | 16 (45.7) | 19 (54.3) | 0 | 22.3 (0.93, | 2.862 (1.007, | 0.0475 |
| (N = 41) | | | | 39.90) | 6.973) | | | | | 43.62) | 8.140) | |
| Rilzabrutinib 400 mg QPM | 12 (31.6) | 26 (68.4) | 0 | 10.5 (−9.62, | 1.672 (0.618, | 0.3039 | 11 (29.7) | 26 (70.3) | 0 | 8.2 (−12.26, | 1.510 (0.535, | 0.4319 |
| (N = 38) | | | | 30.53) | 4.526) | | | | | 28.61) | 4.266) | |
| Placebo (N = 40) | 9 (22.5) | 31 (77.5) | 0 | | | | 8 (22.2) | 28 (77.8) | 0 | | | |
| Week 2 | | | | | | | | | | | | |
| Rilzabrutinib 400 mg TID | 28 (68.3) | 13 (31.7) | 0 | 40.6 (20.49, | 5.982 (2.230, | 0.0003 | 22 (62.9) | 13 (37.1) | 0 | 39.0 (17.40, | 5.505 (1.931, | 0.0012 |
| (N = 41) | | | | 60.75) | 16.042) | | | | | 60.57) | 15.697) | |
| Rilzabrutinib 400 mg BID | 22 (53.7) | 19 (46.3) | 2 (4.9) | 27.8 (7.35, | 3.388 (1.308, | 0.0124 | 18 (51.4) | 17 (48.6) | 1 (2.9) | 28.9 (7.38, | 3.686 (1.307, | 0.0141 |
| (N = 41) | | | | 48.28) | 8.771) | | | | | 50.39) | 10.399) | |
| Rilzabrutinib 400 mg QPM | 15 (39.5) | 23 (60.5) | 0 | 16.6 (−3.87, | 2.169 (0.813, | 0.1176 | 14 (37.8) | 23 (62.2) | 0 | 16.6 (−4.45, | 2.174 (0.790, | 0.1263 |
| (N = 38) | | | | 37.01) | 5.785) | | | | | 37.65) | 5.983) | |
| Placebo (N = 40) | 10 (25.0) | 30 (75.0) | 1 (2.5) | | | | 8 (22.2) | 28 (77.8) | 1 (2.8) | | | |
| Week 3 | | | | | | | | | | | | |
| Rilzabrutinib 400 mg TID | 27 (65.9) | 14 (34.1) | 0 | 31.4 (9.76, | 3.444 (1.387, | 0.0057 | 21 (60.0) | 14 (40.0) | 0 | 27.1 (3.88, | 2.949 (1.124, | 0.0251 |
| (N = 41) | | | | 53.03) | 8.548) | | | | | 50.28) | 7.739) | |
| Rilzabrutinib 400 mg BID | 21 (51.2) | 20 (48.8) | 1 (2.4) | 18.0 (−3.33, | 2.109 (0.854, | 0.1076 | 17 (48.6) | 18 (51.4) | 0 | 17.2 (−5.49, | 2.057 (0.781, | 0.1481 |
| (N = 41) | | | | 39.36) | 5.210) | | | | | 39.84) | 5.418) | |
| Rilzabrutinib 400 mg QPM | 16 (42.1) | 22 (57.9) | 0 | 11.7 (−9.57, | 1.663 (0.650, | 0.2880 | 15 (40.5) | 22 (59.5) | 0 | 11.4 (−10.23, | 1.665 (0.624, | 0.3118 |
| (N = 38) | | | | 32.93) | 4.256) | | | | | 33.01) | 4.445) | |
| Placebo (N = 40) | 13 (32.5) | 27 (67.5) | 1 (2.5) | | | | 11 (30.6) | 25 (69.4) | 1 (2.8) | | | |
| Week 4 | | | | | | | | | | | | |
| Rilzabrutinib 400 mg TID | 29 (70.7) | 12 (29.3) | 3 (7.3) | 29.4 (8.33, | 3.520 (1.371, | 0.0078 | 24 (68.6) | 11 (31.4) | 3 (8.6) | 31.2 (8.87, | 3.855 (1.396, | 0.0083 |
| (N = 41) | | | | 50.37) | 9.039) | | | | | 53.45) | 10.650) | |
| Rilzabrutinib 400 mg BID | 22 (53.7) | 19 (46.3) | 4 (9.8) | 14.7 (−6.60, | 1.853 (0.751, | 0.1892 | 18 (51.4) | 17 (48.6) | 2 (5.7) | 16.8 (−5.71, | 2.046 (0.772, | 0.1587 |
| (N = 41) | | | | 36.10) | 4.572) | | | | | 39.38) | 5.420) | |
| Rilzabrutinib 400 mg QPM | 15 (39.5) | 23 (60.5) | 1 (2.6) | 4.4 (−16.40, | 1.224 (0.469, | 0.6858 | 14 (37.8) | 23 (62.2) | 1 (2.7) | 6.0 (−15.25, | 1.322 (0.487, | 0.5916 |
| (N = 38) | | | | 25.18) | 3.190) | | | | | 27.19) | 3.588) | |
| Placebo (N = 40) | 15 (37.5) | 25 (62.5) | 2 (5.0) | | | | 12 (33.3) | 24 (66.7) | 2 (5.6) | | | |
| Week 5 | | | | | | | | | | | | |
| Rilzabrutinib 400 mg TID | 29 (70.7) | 12 (29.3) | 3 (7.3) | 23.5 (2.40, | 2.703 (1.064, | 0.0352 | 24 (68.6) | 11 (31.4) | 2 (5.7) | 22.4 (−0.46, | 2.512 (0.941, | 0.0629 |
| (N = 41) | | | | 44.63) | 6.869) | | | | | 45.18) | 6.705) | |
| Rilzabrutinib 400 mg BID | 22 (53.7) | 19 (46.3) | 5 (12.2) | 5.2 (−16.49, | 1.238 (0.511, | 0.6393 | 19 (54.3) | 16 (45.7) | 2 (5.7) | 7.0 (−16.15, | 1.329 (0.515, | 0.5596 |
| (N = 41) | | | | 26.99) | 3.003) | | | | | 30.06) | 3.430) | |
| Rilzabrutinib 400 mg QPM | 17 (44.7) | 21 (55.3) | 2 (5.3) | −1.3 (−23.67, | 0.950 (0.390, | 0.9084 | 16 (43.2) | 21 (56.8) | 2 (5.4) | −3.4 (−26.65, | 0.877 (0.354, | 0.7743 |
| (N = 38) | | | | 21.07) | 2.313) | | | | | 19.95) | 2.177) | |
| Placebo (N = 40) | 19 (47.5) | 21 (52.5) | 3 (7.5) | | | | 17 (47.2) | 19 (52.8) | 3 (8.3) | | | |
| Week 6 | | | | | | | | | | | | |
| Rilzabrutinib 400 mg TID | 25 (61.0) | 16 (39.0) | 5 (12.2) | 14.2 (−7.59, | 1.787 (0.727, | 0.2012 | 20 (57.1) | 15 (42.9) | 4 (11.4) | 14.6 (−9.14, | 1.770 (0.690, | 0.2249 |
| (N = 41) | | | | 35.94) | 4.392) | | | | | 38.27) | 4.539) | |
| Rilzabrutinib 400 mg BID | 21 (51.2) | 20 (48.8) | 4 (9.8) | 4.9 (−16.84, | 1.222 (0.505, | 0.6576 | 18 (51.4) | 17 (48.6) | 2 (5.7) | 9.8 (−13.40, | 1.486 (0.577, | 0.4130 |
| (N = 41) | | | | 26.74) | 2.956) | | | | | 32.94) | 3.825) | |
| Rilzabrutinib 400 mg QPM | 16 (42.1) | 22 (57.9) | 4 (10.5) | −1.0 (−22.78, | 0.959 (0.384, | 0.9298 | 15 (40.5) | 22 (59.5) | 4 (10.8) | −0.5 (−22.90, | 0.981 (0.382, | 0.9682 |
| (N = 38) | | | | 20.79) | 2.394) | | | | | 21.98) | 2.519) | |
| Placebo (N = 40) | 18 (45.0) | 22 (55.0) | 6 (15.0) | | | | 15 (41.7) | 21 (58.3) | 5 (13.9) | | | |
| Week 7 | | | | | | | | | | | | |
| Rilzabrutinib 400 mg TID | 25 (61.0) | 16 (39.0) | 6 (14.6) | 18.7 (−1.93, | 2.334 (0.897, | 0.0837 | 22 (62.9) | 13 (37.1) | 5 (14.3) | 22.3 (0.42, | 2.765 (0.982, | 0.0534 |
| (N = 41) | | | | 39.42) | 6.074) | | | | | 44.16) | 7.782) | |
| Rilzabrutinib 400 mg BID | 23 (56.1) | 18 (43.9) | 4 (9.8) | 15.6 (−5.89, | 1.899 (0.774, | 0.1689 | 20 (57.1) | 15 (42.9) | 2 (5.7) | 18.5 (−4.23, | 2.154 (0.819, | 0.1269 |
| (N = 41) | | | | 37.05) | 4.661) | | | | | 41.15) | 5.667) | |
| Rilzabrutinib 400 mg QPM | 16 (42.1) | 22 (57.9) | 6 (15.8) | 3.5 (−18.15, | 1.159 (0.461, | 0.7571 | 15 (40.5) | 22 (59.5) | 6 (16.2) | 2.2 (−20.27, | 1.096 (0.427, | 0.8496 |
| (N = 38) | | | | 25.06) | 2.913) | | | | | 24.65) | 2.814) | |
| Placebo (N = 40) | 16 (40.0) | 24 (60.0) | 5 (12.5) | | | | 14 (38.9) | 22 (61.1) | 4 (11.1) | | | |
| Week 8 | | | | | | | | | | | | |
| Rilzabrutinib 400 mg TID | 27 (65.9) | 14 (34.1) | 8 (19.5) | 23.2 (1.88, | 2.657 (1.048, | 0.0359 | 22 (62.9) | 13 (37.1) | 7 (20.0) | 25.7 (2.65, | 2.815 (1.057, | 0.0307 |
| (N = 41) | | | | 44.51) | 6.735) | | | | | 48.81) | 7.500) | |
| Rilzabrutinib 400 mg BID | 24 (58.5) | 17 (41.5) | 5 (12.2) | 17.5 (−4.45, | 1.985 (0.826, | 0.1221 | 21 (60.0) | 14 (40.0) | 3 (8.6) | 24.0 (0.44, | 2.484 (0.977, | 0.0492 |
| (N = 41) | | | | 39.55) | 4.769) | | | | | 47.47) | 6.318) | |
| Rilzabrutinib 400 mg QPM | 17 (44.7) | 21 (55.3) | 7 (18.4) | 5.3 (−16.20, | 1.257 (0.498, | 0.6342 | 16 (43.2) | 21 (56.8) | 7 (18.9) | 6.6 (−15.50, | 1.331 (0.511, | 0.5608 |
| (N = 38) | | | | 26.83) | 3.172) | | | | | 28.73) | 3.468) | |
| Placebo (N = 40) | 16 (40.0) | 24 (60.0) | 5 (12.5) | | | | 13 (36.1) | 23 (63.9) | 4 (11.1) | | | |
| Week 9 | | | | | | | | | | | | |
| Rilzabrutinib 400 mg TID | 28 (68.3) | 13 (31.7) | 7 (17.1) | 26.2 (5.60, | 3.243 (1.218, | 0.0147 | 23 (65.7) | 12 (34.3) | 6 (17.1) | 29.2 (6.92, | 3.460 (1.235, | 0.0114 |
| (N = 41) | | | | 46.73) | 8.639) | | | | | 51.53) | 9.698) | |
| Rilzabrutinib 400 mg BID | 21 (51.2) | 20 (48.8) | 9 (22.0) | 10.6 (−12.31, | 1.471 (0.633, | 0.3507 | 18 (51.4) | 17 (48.6) | 7 (20.0) | 16.0 (−8.61, | 1.764 (0.720, | 0.1862 |
| (N = 41) | | | | 33.44) | 3.419) | | | | | 40.51) | 4.325) | |
| Rilzabrutinib 400 mg QPM | 18 (47.4) | 20 (52.6) | 6 (15.8) | 8.5 (−12.74, | 1.452 (0.567, | 0.4398 | 17 (45.9) | 20 (54.1) | 6 (16.2) | 9.7 (−12.34, | 1.522 (0.581, | 0.3873 |
| (N = 38) | | | | 29.70) | 3.719) | | | | | 31.72) | 3.985) | |
| Placebo (N = 40) | 16 (40.0) | 24 (60.0) | 6 (15.0) | | | | 13 (36.1) | 23 (63.9) | 5 (13.9) | | | |
| Week 10 | | | | | | | | | | | | |
| Rilzabrutinib 400 mg TID | 27 (65.9) | 14 (34.1) | 7 (17.1) | 15.7 (−5.15, | 2.018 (0.784, | 0.1447 | 23 (65.7) | 12 (34.3) | 5 (14.3) | 19.8 (−2.84, | 2.327 (0.859, | 0.0882 |
| (N = 41) | | | | 36.64) | 5.194) | | | | | 42.45) | 6.308) | |
| Rilzabrutinib 400 mg BID | 21 (51.2) | 20 (48.8) | 10 (24.4) | 2.8 (−19.65, | 1.113 (0.473, | 0.8053 | 18 (51.4) | 17 (48.6) | 8 (22.9) | 7.2 (−16.87, | 1.312 (0.529, | 0.5549 |
| (N = 41) | | | | 25.28) | 2.623) | | | | | 31.33) | 3.255) | |
| Rilzabrutinib 400 mg QPM | 19 (50.0) | 19 (50.0) | 6 (15.8) | 3.5 (−18.12, | 1.159 (0.460, | 0.7599 | 18 (48.6) | 19 (51.4) | 6 (16.2) | 4.1 (−18.19, | 1.190 (0.459, | 0.7267 |
| (N = 38) | | | | 25.03) | 2.918) | | | | | 26.30) | 3.082) | |
| Placebo (N = 40) | 19 (47.5) | 21 (52.5) | 5 (12.5) | | | | 16 (44.4) | 20 (55.6) | 4 (11.1) | | | |
| Week 11 | | | | | | | | | | | | |
| Rilzabrutinib 400 mg TID | 27 (65.9) | 14 (34.1) | 6 (14.6) | 10.3 (−10.11, | 1.624 (0.618, | 0.3217 | 23 (65.7) | 12 (34.3) | 4 (11.4) | 13.6 (−8.65, | 1.839 (0.669, | 0.2206 |
| (N = 41) | | | | 30.69) | 4.267) | | | | | 35.92) | 5.054) | |
| Rilzabrutinib 400 mg BID | 22 (53.7) | 19 (46.3) | 9 (22.0) | 0.2 (−22.10, | 1.009 (0.426, | 0.9836 | 19 (54.3) | 16 (45.7) | 7 (20.0) | 4.4 (−19.56, | 1.183 (0.474, | 0.7166 |
| (N = 41) | | | | 22.56) | 2.389) | | | | | 28.37) | 2.949) | |
| Rilzabrutinib 400 mg QPM | 19 (50.0) | 19 (50.0) | 7 (18.4) | −1.3 (−22.73, | 0.945 (0.373, | 0.9077 | 18 (48.6) | 19 (51.4) | 7 (18.9) | −1.3 (−23.40, | 0.946 (0.363, | 0.9118 |
| (N = 38) | | | | 20.13) | 2.395) | | | | | 20.84) | 2.465) | |
| Placebo (N = 40) | 21 (52.5) | 19 (47.5) | 5 (12.5) | | | | 18 (50.0) | 18 (50.0) | 4 (11.1) | | | |
| Week 12 | | | | | | | | | | | | |
| Rilzabrutinib 400 mg TID | 23 (56.1) | 18 (43.9) | 11 (26.8) | 11.7 (−9.78, | 1.638 (0.660, | 0.2871 | 20 (57.1) | 15 (42.9) | 8 (22.9) | 14.0 (−8.89, | 1.805 (0.681, | 0.2342 |
| (N = 41) | | | | 33.27) | 4.062) | | | | | 36.90) | 4.784) | |
| Rilzabrutinib 400 mg BID | 18 (43.9) | 23 (56.1) | 14 (34.1) | 1.0 (−21.02, | 1.041 (0.434, | 0.9295 | 15 (42.9) | 20 (57.1) | 11 (31.4) | 1.7 (−21.77, | 1.069 (0.420, | 0.8897 |
| (N = 41) | | | | 23.03) | 2.493) | | | | | 25.12) | 2.719) | |
| Rilzabrutinib 400 mg QPM | 14 (36.8) | 24 (63.2) | 10 (26.3) | −4.0 (−25.52, | 0.841 (0.333, | 0.7203 | 13 (35.1) | 24 (64.9) | 10 (27.0) | −5.8 (−27.91, | 0.780 (0.300, | 0.6176 |
| (N = 38) | | | | 17.49) | 2.125) | | | | | 16.37) | 2.029) | |
| Placebo (N = 40) | 17 (42.5) | 23 (57.5) | 7 (17.5) | | | | 15 (41.7) | 21 (58.3) | 5 (13.9) | | | |

ªOR: odds ratio; RRD: response rate difference; derived from the Mantel-Haenszel estimator
^{b}CMH test was performed on the association between the responder status and intervention group, stratified by region.
Note:
Participants who received selected prohibited/rescue medications (high/medium impact on efficacy confirmed through blinded medical review) before the given week were considered as non-responders, and participants with missing data at the given week were considered as non-responders up to Week 12.

### ISS7

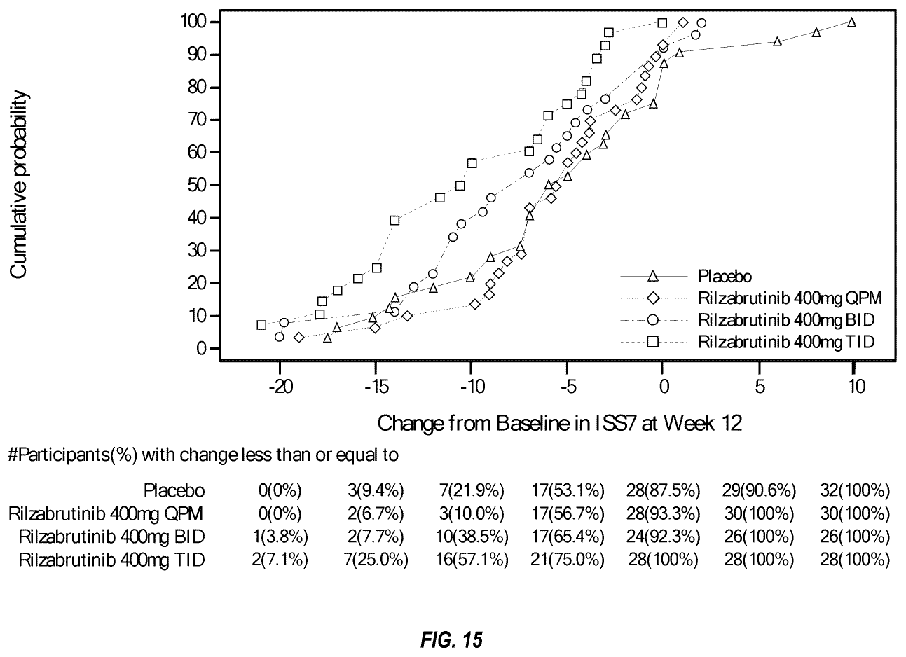

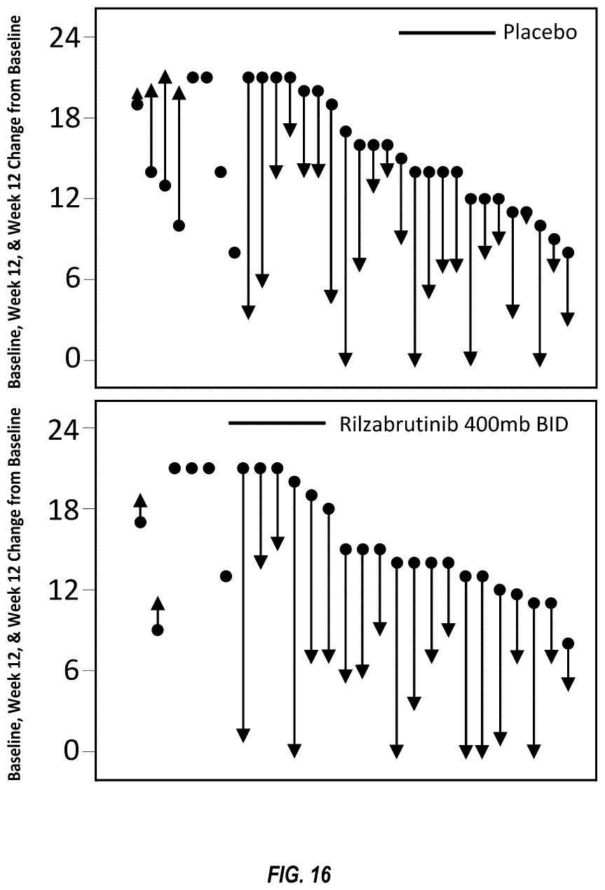

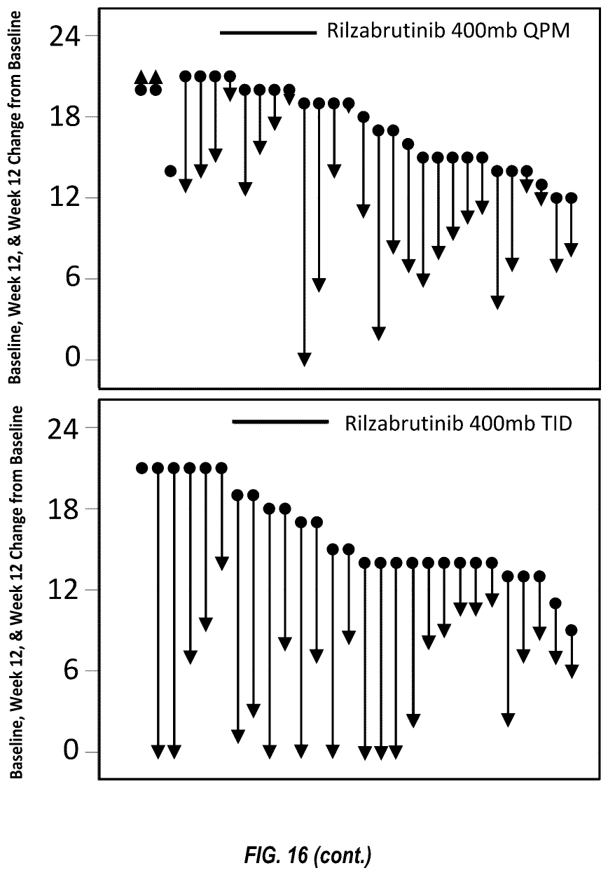

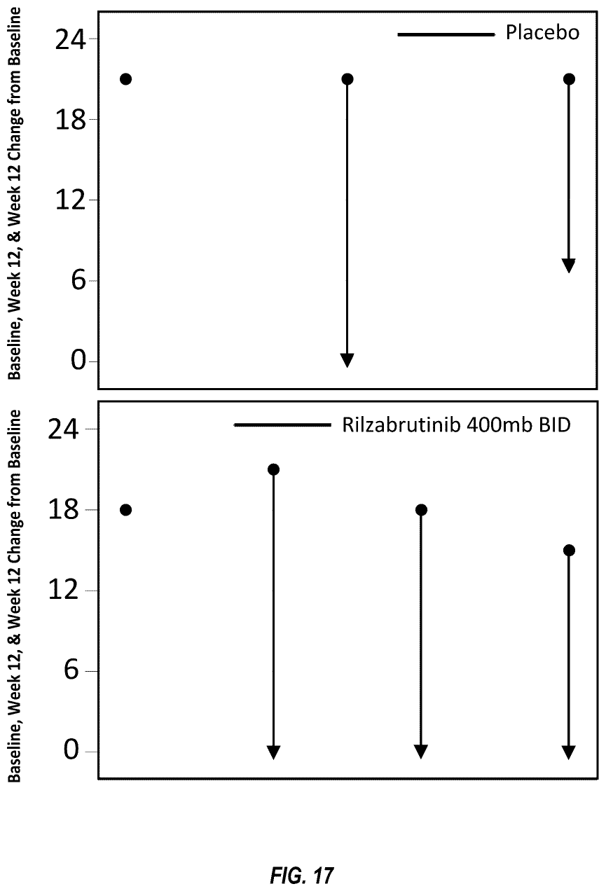

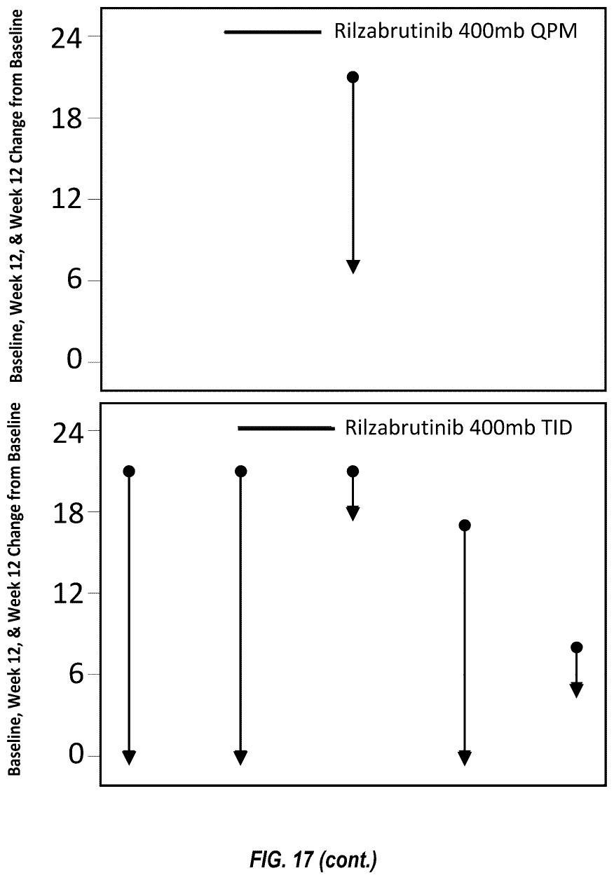

Rilzabrutinib treatment led to significant improvements in ISS7. Notably, participants receiving 400 mg TID rilzabrutinib exhibited a significant reduction in ISS7 at Week 12, thereby meeting the primary endpoint in the United States and United States Reference Countries (see FIGS. 15-17 and Tables 24-26). In the ITT population, participants receiving 400 mg TID rilzabrutinib exhibited a LS mean change from baseline at Week 12 of −9.58 (p=0.0181) points, while patients receiving the placebo exhibited a LS mean change of −6.31 points (see Table 24). Similar results were observed for the omalizumab-naïve population, both prior to (LS mean change from baseline of −9.21, p=0.0168; see Table 25) and following (LS mean change from baseline of −9.57, p=0.0078; see Table 26) the removal of the asymptomatic outlier (UAS7/ISS7=0 at baseline) who was erroneously randomized into the 400 mg TID rilzabrutinib arm.

In the 400 mg TID arm, all patients except one showed improvement in the weekly ISS7. Moreover, a clear separation was observed between patients receiving 400 mg TID rilzabrutinib and patients receiving the placebo across all response thresholds (see FIG. 15).

Further analysis of the omalizumab-naïve population supports the efficacy of rilzabrutinib treatment in CSU (see Table 27). Overall, baseline participant demographics did not appear to affect the efficacy of 400 mg TID rilzabrutinib Moreover, 400 mg TID rilzabrutinib treatment was efficacious in patients with characteristics such as low IgE, a positive BHRA status, and a history of angioedema.

TABLE 24
Change from baseline in ISS7 at Week 12 (ITT population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| ISS7 | (N = 40) | (N = 38) | (N = 41) | (N = 41) |

Baseline

| Number | 40 | | 38 | | 41 | | 41 | |
|---|---|---|---|---|---|---|---|---|
| Mean (SD) | 15.60 | (4.24) | 16.50 | (3.53) | 15.80 | (3.68) | 15.88 | (4.37) |

| | | | | |
|---|---|---|---|---|
| Median | 14.50 | 16.50 | 15.00 | 15.00 |
| Q1; Q3 | 12.00; 20.50 | 14.00; 20.00 | 13.00; 19.00 | 14.00; 20.00 |
| Min; Max | 8.0; 21.0 | 9.0; 21.0 | 8.0; 21.0 | 0.0; 21.0 |

Week 12

| | | | | | | | | |
|---|---|---|---|---|---|---|---|---|
| Number (observed/imputed) | 35 | (33/2) | 31 | (28/3) | 30 | (27/3) | 33 | (30/3) |
| Mean (SD) | 9.96 | (7.02) | 11.37 | (5.51) | 7.61 | (7.18) | 5.59 | (5.58) |

| | | | | |
|---|---|---|---|---|
| Median | 8.00 | 11.20 | 7.00 | 6.00 |
| Q1; Q3 | 4.67; 14.00 | 7.00; 15.17 | 0.00; 13.00 | 0.00; 8.75 |
| Min; Max | 0.0; 21.0 | 0.0; 21.0 | 0.0; 21.0 | 0.0; 21.0 |

Change from baseline

| Number (observed/imputed) | 35 | (33/2) | 31 | (28/3) | 30 | (27/3) | 33 | (30/3) |
|---|---|---|---|---|---|---|---|---|
| Mean (SD) | −5.67 | (7.15) | −5.95 | (4.87) | −8.21 | (6.76) | −10.68 | (6.50) |
| Median | −6.00 | | −5.67 | | −8.00 | | −10.67 | |
| Q1;Q3 | −10.00; 0.00 | | −8.60; −1.40 | | −13.00; −3.00 | | −16.00; −4.25 | |
| Min; Max | −21.0; 9.8 | | −19.0; 1.0 | | −21.0; 2.0 | | −21.0; 0.0 | |
| LS Mean (SE) ^{a} | −6.31 | (1.02) | −5.46 | (1.03) | −8.06 | (1.02) | −9.58 | (1.01) |
| LS Mean Diff vs. placebo (95% CI) ^{a} | | | 0.85 | (−1.90, 3.60) | −1.75 | (−4.46, 0.95) | −3.26 | (−5.97, −0.56) |

| | | | |
|---|---|---|---|
| P-value vs. placebo ^{a} | 0.5439 | 0.2041 | 0.0181 |

^{a} Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.

TABLE 25
Primary Analysis: Change from Baseline in ISS7 at Week 12 (Omalizumab-Naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| ISS7 | (N = 36) | (N = 37) | (N = 35) | (N = 35) |

Baseline

| Number | 36 | | 37 | | 35 | | 35 | |
|---|---|---|---|---|---|---|---|---|
| Mean (SD) | 15.14 | (4.16) | 16.38 | (3.50) | 15.45 | (3.72) | 15.69 | (4.26) |

| | | | | |
|---|---|---|---|---|
| Median | 14.00 | 16.00 | 14.00 | 14.00 |
| Q1; Q3 | 12.00; 19.50 | 14.00; 20.00 | 13.00; 19.00 | 14.00; 19.00 |
| Min; Max | 8.0; 21.0 | 9.0; 21.0 | 8.0; 21.0 | 0.0; 21.0 |

Week 12

| | | | | | | | | |
|---|---|---|---|---|---|---|---|---|
| Number (observed/imputed) | 32 | (31/1) | 30 | (27/3) | 26 | (24/2) | 28 | (27/1) |
| Mean (SD) | 10.02 | (6.82) | 11.51 | (5.54) | 8.09 | (6.95) | 5.77 | (5.26) |

| | | | | |
|---|---|---|---|---|
| Median | 8.50 | 11.60 | 7.00 | 7.00 |
| Q1; Q3 | 4.83; 14.00 | 7.00; 15.17 | 1.17; 13.00 | 0.00; 8.88 |
| Min; Max | 0.0; 21.0 | 0.0; 21.0 | 0.0; 21.0 | 0.0; 21.0 |

Change from baseline

| Number (observed/imputed) | 32 | (31/1) | 30 | (27/3) | 26 | (24/2) | 28 | (27/1) |
|---|---|---|---|---|---|---|---|---|
| Mean (SD) | −5.11 | (6.70) | −5.69 | (4.72) | −7.40 | (6.12) | −10.27 | (6.02) |
| Median | −5.50 | | −5.33 | | −7.00 | | −10.33 | |
| Q1; Q3 | −9.00; −0.25 | | −8.17; −1.40 | | −11.00; −3.00 | | −14.50; −4.63 | |
| Min; Max | −17.5; 9.8 | | −19.0; 1.0 | | −20.0; 2.0 | | −21.0; 0.0 | |
| LS Mean (SE) ^{a} | −5.77 | (1.05) | −5.19 | (1.02) | −7.73 | (1.06) | −9.21 | (1.05) |
| LS Mean Diff vs. placebo (95% CI) ^{a} | | | 0.58 | (−2.24, 3.40) | −1.96 | (−4.77, 0.86) | −3.44 | (−6.25, −0.62) |

| | | | |
|---|---|---|---|
| P-value vs. placebo ^{a} | 0.6869 | 0.1728 | 0.0168 |

^{a} Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.

TABLE 26
Change from Baseline in ISS7 at Week 12 (Omalizumab-Naïve Population with Outlier Removed from Analysis)

| | | | | |
|---|---|---|---|---|
| | | Rilzabrutinib | Rilzabrutinib | Rilzabrutinib |
| | Placebo | 400 mg QPM | 400 mg BID | 400 mg TID |
| ISS7 | (N = 36) | (N = 37) | (N = 35) | (N = 34) |

Baseline

| Number | 36 | | 37 | | 35 | | 34 | |
|---|---|---|---|---|---|---|---|---|
| Mean (SD) | 15.14 | (4.16) | 16.38 | (3.50) | 15.45 | (3.72) | 16.15 | (3.32) |

| | | | | |
|---|---|---|---|---|
| Median | 14.00 | 16.00 | 14.00 | 14.50 |
| Q1; Q3 | 12.00; 19.50 | 14.00; 20.00 | 13.00; 19.00 | 14.00; 19.00 |
| Min; Max | 8.0; 21.0 | 9.0; 21.0 | 8.0; 21.0 | 9.0; 21.0 |

Week 12

| | | | | | | | | |
|---|---|---|---|---|---|---|---|---|
| Number (observed/imputed) | 32 | (31/1) | 30 | (27/3) | 26 | (24/2) | 28 | (27/1) |
| Mean (SD) | 10.02 | (6.82) | 11.51 | (5.54) | 8.09 | (6.95) | 5.77 | (5.26) |

| | | | | |
|---|---|---|---|---|
| Median | 8.50 | 11.60 | 7.00 | 7.00 |
| Q1; Q3 | 4.83; 14.00 | 7.00; 15.17 | 1.17; 13.00 | 0.00; 8.88 |
| Min; Max | 0.0; 21.0 | 0.0; 21.0 | 0.0; 21.0 | 0.0; 21.0 |

Change from baseline

| Number (observed/imputed) | 32 | (31/1) | 30 | (27/3) | 26 | (24/2) | 28 | (27/1) |
|---|---|---|---|---|---|---|---|---|
| Mean (SD) | −5.11 | (6.70) | −5.69 | (4.72) | −7.40 | (6.12) | −10.27 | (6.02) |
| Median | −5.50 | | −5.33 | | −7.00 | | −10.33 | |
| Q1; Q3 | −9.00; −0.25 | | −8.17; −1.40 | | 11.00; −3.00 | | −14.50; − 4.63 | |
| Min; Max | −17.5; 9.8 | | −19.0; 1.0 | | −20.0; 2.0 | | −21.0; 0.0 | |
| LS Mean (SE) ^{a} | −5.71 | (1.02) | −5.29 | (1.01) | −7.76 | (1.06) | −9.57 | (1.06) |
| LS Mean Diff vs. placebo (95% CI) ^{a} | | | 0.42 | (−2.33, 3.18) | −2.05 | (−4.84, 0.74) | −3.86 | (−6.71, −1.02) |

| | | | |
|---|---|---|---|
| P-value vs. placebo ^{a} | 0.7634 | 0.1491 | 0.0078 |

^{a} Each of the imputed complete data were analyzed by fitting an ANCOVA model with the corresponding baseline value, intervention group and region as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.
One patient was randomized to Rilzabrutinib 400 mg TID group by mistake (baseline ISS7/UAS7 = 0). Removed this participant from the analysis.

TABLE 27
Subgroup Analysis: Change from Baseline in ISS7 at Week 12 by Subgroups (Omalizumab-naïve population)

| | | | |
|---|---|---|---|
| | Rilzabrutinib | Rilzabrutinib | Rilzabrutinib |
| Placebo | 400 mg QPM | 400 mg BID | 400 mg TID |
| (N = 36) | (N = 37) | (N = 35) | (N = 35) |

Age group (years)

| <44 (Median) | | | | | | | | |
|---|---|---|---|---|---|---|---|---|
| Number (observed/imputed) | 21 | (20/1) | 16 | (14/2) | 12 | (12/0) | 8 | (8/0) |
| Mean (SD) | −4.31 | (6.69) | −5.45 | (5.57) | −5.97 | (4.65) | −10.53 | (5.94) |
| LS Mean (SE) ^{a} | −4.47 | (1.30) | −4.68 | (1.40) | −7.05 | (1.52) | −9.04 | (1.89) |
| LS Mean Diff vs. placebo (95% CI) ^{a} | | | −0.21 | (−4.03, 3.62) | −2.58 | (−6.51, 1.36) | −4.56 | (−9.03, −0.09) |
| P-value vs. placebo ^{a} | | | 0.9161 | | 0.1987 | | 0.0454 | |
| ≥44 (Median) | | | | | | | | |
| Number (observed/imputed) | 11 | (11/0) | 14 | (13/1) | 14 | (12/2) | 20 | (19/1) |
| Mean (SD) | −6.64 | (6.75) | −5.95 | (3.70) | −8.62 | (7.08) | −10.16 | (6.20) |
| LS Mean (SE) ^{a} | −6.60 | (1.68) | −4.77 | (1.45) | −8.10 | (1.40) | −9.26 | (1.23) |
| LS Mean Diff vs. placebo (95% CI) ^{a} | | | 1.83 | (−2.53, 6.19) | −1.50 | (−5.80, 2.80) | −2.66 | (−6.72, 1.41) |

| | | | |
|---|---|---|---|
| P-value vs. placebo ^{a} | 0.4100 | 0.4943 | 0.2007 |
| Overall p-value for interaction ^{b} | | | 0.9294 |

Age group (years)

| <65 | | | | | | | | |
|---|---|---|---|---|---|---|---|---|
| Number (observed/imputed) | 30 | (29/1) | 29 | (26/3) | 25 | (23/2) | 24 | (23/1) |
| Mean (SD) | −5.52 | (6.47) | −5.68 | (4.80) | −7.17 | (6.13) | −9.73 | (5.71) |
| LS Mean (SE) ^{a} | −5.66 | (1.04) | −4.78 | (1.01) | −7.37 | (1.05) | −8.63 | (1.09) |
| LS Mean Diff vs. placebo (95% CI) ^{a} | | | 0.88 | (−2.00, 3.75) | −1.71 | (−4.59, 1.18) | −2.97 | (−5.90, −0.03) |
| P-value vs. placebo ^{a} | | | 0.5497 | | 0.2453 | | 0.0478 | |
| ≥65 | | | | | | | | |
| Number (observed/imputed) | 2 | (2/0) | 1 | (1/0) | 1 | (1/0) | 4 | (4/0) |
| Mean (SD) | 1.00 | (9.90) | −5.83 | (NC) | −13.00 | (NC) | −13.50 | (7.71) |
| LS Mean (SE) ^{a} | 0.90 | (3.84) | 2.48 | (6.23) | −13.32 | (4.62) | −12.67 | (2.73) |
| LS Mean Diff vs. placebo (95% CI) ^{a} | | | 1.58 | (−12.80, 15.96) | −14.22 | (−25.98, −2.46) | − 13.57 | (−22.81, −4.32) |

| | | | |
|---|---|---|---|
| P-value vs. placebo ^{a} | 0.8295 | 0.0178 | 0.0040 |
| Overall p-value for interaction ^{b} | | | 0.1940 |

Gender

| Male | | | | | | | | |
|---|---|---|---|---|---|---|---|---|
| Number (observed/imputed) | 11 | (11/0) | 8 | (8/0) | 9 | (8/1) | 5 | (5/0) |
| Mean (SD) | −3.50 | (7.05) | −5.20 | (5.77) | −8.17 | (6.82) | −7.08 | (3.25) |
| LS Mean (SE) ^{a} | −3.55 | (1.87) | −4.68 | (2.21) | −8.42 | (1.87) | −6.50 | (2.65) |
| LS Mean Diff vs. placebo (95% CI) ^{a} | | | −1.14 | (−6.82, 4.55) | −4.88 | (−9.99, 0.23) | −2.95 | (−9.38, 3.48) |
| P-value vs. placebo ^{a} | | | 0.6946 | | 0.0613 | | 0.3683 | |
| Female | | | | | | | | |
| Number (observed/imputed) | 21 | (20/1) | 22 | (19/3) | 17 | (16/1) | 23 | (22/1) |
| Mean (SD) | −5.95 | (6.52) | −5.86 | (4.42) | −6.99 | (5.89) | −10.96 | (6.30) |
| LS Mean (SE) ^{a} | −6.30 | (1.20) | 4.77 | (1.12) | −6.92 | (1.24) | −9.80 | (1.09) |
| LS Mean Diff vs. placebo (95% CI) a | | | 1.52 | (−1.76, 4.81) | −0.62 | (−4.00, 2.75) | −3.50 | (−6.68, −0.33) |

| | | | |
|---|---|---|---|
| P-value vs. placebo ^{a} | 0.3627 | 0.7173 | 0.0305 |
| Overall p-value for interaction ^{b} | | | 0.3883 |

Baseline weight (kg)

| <72.9 (Median) | | | | | | | | |
|---|---|---|---|---|---|---|---|---|
| Number (observed/imputed) | 18 | (18/0) | 17 | (16/1) | 16 | (15/1) | 13 | (13/0) |
| Mean (SD) | −5.88 | (6.08) | −4.51 | (3.00) | −6.55 | (5.45) | −11.22 | (7.22) |
| LS Mean (SE) ^{a} | −5.75 | (1.29) | −3.98 | (1.27) | −6.60 | (1.34) | −9.97 | (1.47) |
| LS Mean Diff vs. placebo (95% CI) ^{a} | | | 1.77 | (−1.80, 5.35) | −0.84 | (−4.46, 2.77) | −4.22 | (−8.04, −0.39) |
| P-value vs. placebo ^{a} | | | 0.3311 | | 0.6472 | | 0.0306 | |
| ≥72.9 (Median) | | | | | | | | |
| Number (observed/imputed) | 14 | (13/1) | 13 | (11/2) | 10 | (9/1) | 15 | (14/1) |
| Mean (SD) | −4.12 | (7.53) | −7.22 | (6.10) | −8.75 | (7.15) | −9.44 | (4.85) |
| LS Mean (SE) ^{a} | −4.65 | (1.54) | −5.86 | (1.55) | −8.28 | (1.53) | −8.80 | (1.38) |
| LS Mean Diff vs. placebo (95% CI) ^{a} | | | −1.21 | (−5.49, 3.07) | −3.62 | (−7.92, 0.67) | −4.15 | (−8.21, −0.09) |

| | | | |
|---|---|---|---|
| P-value vs. placebo ^{a} | 0.5791 | 0.0983 | 0.0451 |
| Overall p-value for interaction ^{b} | | | 0.4436 |

Baseline weight (kg)

| <60 | | | | | | | | |
|---|---|---|---|---|---|---|---|---|
| Number (observed/imputed) | 10 | (10/0) | 6 | (5/1) | 7 | (7/0) | 8 | (8/0) |
| Mean (SD) | −4.70 | (5.59) | −4.71 | (2.89) | −5.29 | (4.80) | −11.17 | (7.60) |
| LS Mean (SE) ^{a} | −4.34 | (1.79) | −4.12 | (2.29) | −5.70 | (2.01) | −11.07 | (2.04) |
| LS Mean Diff vs. placebo (95% CI) ^{a} | | | 0.22 | (−5.52, 5.95) | −1.37 | (−6.59, 3.86) | 6.74 | (−12.06, −1.41) |
| P-value vs. placebo ^{a} | | | 0.9410 | | 0.6083 | | 0.0131 | |
| ≥60-<90 | | | | | | | | |
| Number (observed/imputed) | 15 | (14/1) | 21 | (19/2) | 16 | (14/2) | 17 | (17/0) |
| Mean (SD) | −5.69 | (7.04) | −5.28 | (4.97) | −7.71 | (6.96) | −9.91 | (5.71) |
| LS Mean (SE) ^{a} | −6.06 | (1.42) | −4.36 | (1.15) | −7.08 | (1.35) | −8.41 | (1.24) |
| LS Mean Diff vs. placebo (95% CI) ^{a} | | | 1.71 | (−1.90, 5.31) | −1.01 | (−4.85, 2.82) | −2.35 | (−6.03, 1.34) |
| P-value vs. placebo ^{a} | | | 0.3530 | | 0.6049 | | 0.2123 | |
| ≥90 | | | | | | | | |
| Number (observed/imputed) | 7 | (7/0) | 3 | (3/0) | 3 | (3/0) | 3 | (2/1) |
| Mean (SD) | −4.45 | (8.22) | −10.47 | (4.01) | −10.67 | (1.53) | −9.89 | (4.55) |
| LS Mean (SE) ^{a} | −5.14 | (2.38) | −8.76 | (3.75) | −10.60 | (2.52) | −10.90 | (3.63) |
| LS Mean Diff vs. placebo (95% CI) ^{a} | | | −3.62 | (−12.56, 5.32) | −5.46 | (−12.33, 1.40) | −5.76 | (−14.13, 2.61) |

| | | | |
|---|---|---|---|
| P-value vs. placebo ^{a} | 0.4278 | 0.1187 | 0.1775 |
| Overall p-value for interaction ^{b} | | | 0.7163 |

Baseline BMI (kg/m²)

| <25 | | | | | | | | |
|---|---|---|---|---|---|---|---|---|
| Number (observed/imputed) | 15 | (15/0) | 14 | (13/1) | 17 | (16/1) | 11 | (11/0) |
| Mean (SD) | −4.50 | (5.63) | −4.18 | (3.14) | −7.34 | (6.20) | −9.13 | (7.05) |
| LS Mean (SE) ^{a} | −4.42 | (1.40) | −3.64 | (1.42) | −7.22 | (1.29) | −9.17 | (1.68) |
| LS Mean Diff vs. placebo (95% CI) ^{a} | | | 0.77 | (−3.14, 4.68) | −2.81 | (−6.51, 0.90) | −4.75 | (−9.04, −0.47) |
| P-value vs. placebo ^{a} | | | 0.6983 | | 0.1382 | | 0.0297 | |
| >25-<30 | | | | | | | | |
| Number (observed/imputed) | 9 | (8/1) | 7 | (6/1) | 5 | (4/1) | 13 | (13/0) |
| Mean (SD) | −3.39 | (8.46) | −5.60 | (6.96) | −7.10 | (7.55) | −10.74 | (5.51) |
| LS Mean (SE) ^{a} | −5.55 | (1.96) | −3.43 | (1.97) | −6.90 | (2.41) | −8.43 | (1.46) |
| LS Mean Diff vs. placebo (95% CI) ^{a} | | | 2.13 | (−3.54, 7.80) | −1.35 | (−7.44, 4.74) | −2.88 | (−7.76, 2.00) |
| P-value vs. placebo ^{a} | | | 0.4619 | | 0.6647 | | 0.2473 | |
| ≥30 | | | | | | | | |
| Number (observed/imputed) | 8 | (8/0) | 9 | (8/1) | 4 | (4/0) | 4 | (3/1) |
| Mean (SD) | −8.19 | (6.16) | −8.09 | (4.22) | −8.00 | (5.48) | −11.88 | (5.44) |
| LS Mean (SE) ^{a} | −7.67 | (2.06) | −7.49 | (1.92) | −8.24 | (2.10) | −10.85 | (2.59) |
| LS Mean Diff vs. placebo (95% CI) ^{a} | | | 0.17 | (−5.29, 5.63) | −0.57 | (−6.35, 5.21) | −3.19 | (−9.64, 3.26) |

| | | | |
|---|---|---|---|
| P-value vs. placebo ^{a} | 0.9508 | 0.8467 | 0.3327 |
| Overall p-value for interaction ^{b} | | | 0.9842 |

Region ^{c}

| Asia | | | | | | | | |
|---|---|---|---|---|---|---|---|---|
| Number (observed/imputed) | 8 | (8/0) | 9 | (9/0) | 8 | (8/0) | 6 | (6/0) |
| Mean (SD) | −3.46 | (5.86) | −5.66 | (5.13) | −6.86 | (5.22) | −8.21 | (6.23) |
| LS Mean (SE) a | −3.45 | (1.91) | −5.17 | (1.81) | −6.99 | (1.97) | −8.33 | (2.09) |
| LS Mean Diff vs. placebo (95% CI) ^{a} | | | −1.72 | (−6.88, 3.45) | −3.54 | (−8.93, 1.85) | −4.87 | (−10.41, 0.67) |
| P-value vs. placebo ^{a} | | | 0.5148 | | 0.1978 | | 0.0848 | |
| Latin America | | | | | | | | |
| Number (observed/imputed) | 11 | (10/1) | 10 | (7/3) | 8 | (6/2) | 6 | (6/0) |
| Mean (SD) | −7.29 | (7.65) | −6.51 | (5.89) | −4.42 | (6.21) | −7.34 | (5.81) |
| LS Mean (SE) ^{a} | −6.59 | (1.75) | −5.19 | (1.62) | −4.75 | (1.94) | −6.07 | (2.20) |
| LS Mean Diff vs. placebo (95% CI) ^{a} | | | 1.40 | (−3.23, 6.04) | 1.84 | (−3.27, 6.94) | 0.52 | (−4.93, 5.96) |
| P-value vs. placebo ^{a} | | | 0.5532 | | 0.4802 | | 0.8521 | |
| Eastern Europe | | | | | | | | |
| Number (observed/imputed) | 2 | (2/0) | 4 | (4/0) | 3 | (3/0) | 4 | (4/0) |
| Mean (SD) | −6.00 | (8.49) | −5.54 | (3.34) | −10.81 | (7.84) | −13.17 | (6.21) |
| LS Mean (SE) ^{a} | −12.67 | (4.21) | −4.74 | (2.49) | −4.38 | (4.01) | −14.02 | (2.49) |
| LS Mean Diff vs. placebo (95% CI) ^{a} | | | 7.93 | (−2.07, 17.93) | 8.28 | (−5.64, 22.21) | −1.35 | (−10.49, 7.79) |
| P-value vs. placebo ^{a} | | | 0.1201 | | 0.2436 | | 0.7720 | |
| Western Countries | | | | | | | | |
| Number (observed/imputed) | 11 | (11/0) | 7 | (7/0) | 7 | (7/0) | 12 | (11/1) |
| Mean (SD) | −3.97 | (6.35) | −4.61 | (3.50) | 9.95 | (5.74) | −11.79 | (5.72) |
| LS Mean (SE) ^{a} | −4.55 | (1.70) | −3.02 | (2.04) | −10.12 | (1.65) | 9.58 | (1.54) |
| LS Mean Diff vs. placebo (95% CI) ^{a} | | | 1.53 | (−3.74, 6.79) | −5.57 | (−10.21, −0.93) | −5.03 | (−9.55, −0.51) |

| | | | |
|---|---|---|---|
| P-value vs. placebo ^{a} | 0.5697 | 0.0186 | 0.0293 |
| Overall p-value for interaction ^{b} | | | 0.3768 |

Race

| White | | | | | | | | |
|---|---|---|---|---|---|---|---|---|
| Number (observed/imputed) | 24 | (23/1) | 21 | (18/3) | 18 | (16/2) | 20 | (19/1) |
| Mean (SD) | −5.66 | (6.98) | −5.70 | (4.66) | −7.64 | (6.60) | −10.88 | (6.28) |
| LS Mean (SE) ^{a} | −6.01 | (1.20) | −4.41 | (1.21) | −7.91 | (1.21) | −9.18 | (1.24) |
| LS Mean Diff vs. placebo (95% CI) ^{a} | | | 1.60 | (−1.77, 4.97) | −1.90 | (−5.24, 1.43) | −3.17 | (−6.56, 0.21) |
| P-value vs. placebo ^{a} | | | 0.3527 | | 0.2635 | | 0.0663 | |
| All the Others | | | | | | | | |
| Number (observed/imputed) | 8 | (8/0) | 9 | (9/0) | 8 | (8/0) | 8 | (8/0) |
| Mean (SD) | −3.46 | (5.86) | −5.66 | (5.13) | −6.86 | (5.22) | −8.74 | (5.36) |
| LS Mean (SE) ^{a} | −3.47 | (1.86) | −4.94 | (1.72) | −6.99 | (1.92) | −8.74 | (1.80) |
| LS Mean Diff vs. placebo (95% CI) ^{a} | | | −1.48 | (−6.47, 3.52) | −3.52 | (−8.76, 1.71) | −5.27 | (−10.33, −0.21) |

| | | | |
|---|---|---|---|
| P-value vs. placebo ^{a} | 0.5627 | 0.1874 | 0.0414 |
| Overall p-value for interaction ^{b} | | | 0.8028 |

Ethnicity

| Hispanic | | | | | | | | |
|---|---|---|---|---|---|---|---|---|
| Number (observed/imputed) | 10 | (10/0) | 12 | (10/2) | 8 | (6/2) | 8 | (8/0) |
| Mean (SD) | −8.02 | (7.65) | −5.04 | (5.25) | −4.42 | (6.21) | −8.13 | (6.19) |
| LS Mean (SE) ^{a} | −7.01 | (1.85) | −4.19 | (1.52) | −5.19 | (1.87) | 7.36 | (1.94) |
| LS Mean Diff vs. placebo (95% CI) ^{a} | | | 2.82 | (−1.84, 7.47) | 1.82 | (−3.35, 6.98) | −0.35 | (−5.55, 4.84) |
| P-value vs. placebo ^{a} | | | 0.2356 | | 0.4911 | | 0.8936 | |
| Non-Hispanic | | | | | | | | |
| Number (observed/imputed) | 22 | (21/1) | 18 | (17/1) | 17 | (17/0) | 18 | (17/1) |
| Mean (SD) | −3.79 | (5.94) | −6.12 | (4.43) | −8.59 | (5.90) | −11.83 | (5.76) |
| LS Mean (SE) ^{a} | −4.47 | (1.21) | −5.29 | (1.31) | −8.43 | (1.26) | −10.16 | (1.27) |
| LS Mean Diff vs. placebo (95% CI) ^{a} | | | −0.82 | (−4.37, 2.73) | −3.96 | (−7.39, −0.53) | −5.69 | (−9.13, −2.25) |

| | | | |
|---|---|---|---|
| P-value vs. placebo ^{a} | 0.6503 | 0.0236 | 0.0012 |
| Overall p-value for interaction ^{b} | | | 0.2235 |

History of angioedema

| Yes | | | | | | | | |
|---|---|---|---|---|---|---|---|---|
| Number (observed/imputed) | 15 | (14/1) | 11 | (8/3) | 8 | (6/2) | 9 | (8/1) |
| Mean (SD) | −5.34 | (6.25) | −6.31 | (5.88) | −4.04 | (5.66) | −9.01 | (5.02) |
| LS Mean (SE) ^{a} | −5.25 | (1.51) | −5.00 | (1.53) | −6.26 | (1.90) | −8.24 | (1.90) |
| LS Mean Diff vs. placebo (95% CI) ^{a} | | | 0.25 | (−3.98, 4.47) | −1.01 | (−5.77, 3.76) | −2.99 | (−7.72, 1.75) |
| P-value vs. placebo ^{a} | | | 0.9094 | | 0.6793 | | 0.2160 | |
| No | | | | | | | | |
| Number (observed/imputed) | 17 | (17/0) | 19 | (19/0) | 18 | (18/0) | 19 | (19/0) |
| Mean (SD) | −4.90 | (7.26) | −5.32 | (4.04) | −8.89 | (5.85) | −10.86 | (6.47) |
| LS Mean (SE) ^{a} | −5.34 | (1.39) | −4.56 | (1.34) | −7.93 | (1.24) | −9.56 | (1.25) |
| LS Mean Diff vs. placebo (95% CI) ^{a} | | | 0.78 | (−3.03, 4.59) | −2.59 | (−6.23, 1.05) | −4.22 | (−7.88, −0.56) |

| | | | |
|---|---|---|---|
| P-value vs. placebo ^{a} | 0.6884 | 0.1626 | 0.0239 |
| Overall p-value for interaction ^{b} | | | 0.9105 |

Baseline UAS7 score

| <28 | | | | | | | | |
|---|---|---|---|---|---|---|---|---|
| Number (observed/imputed) | 12 | (12/0) | 6 | (6/0) | 11 | (9/2) | 11 | (11/0) |
| Mean (SD) | −2.68 | (6.50) | −5.32 | (3.03) | −6.29 | (4.93) | −7.61 | (4.20) |
| LS Mean (SE) ^{a} | −3.17 | (1.42) | −3.33 | (1.83) | −5.40 | (1.42) | −6.43 | (1.53) |
| LS Mean Diff vs. placebo (95% CI) ^{a} | | | −0.15 | (−4.72, 4.41) | −2.22 | (−6.23, 1.79) | −3.26 | (−7.39, 0.88) |
| P-value vs. placebo ^{a} | | | 0.9472 | | 0.2773 | | 0.1225 | |
| ≥28 | | | | | | | | |
| Number (observed/imputed) | 20 | (19/1) | 24 | (21/3) | 15 | (15/0) | 17 | (16/1) |
| Mean (SD) | −6.57 | (6.54) | −5.78 | (5.10) | −8.21 | (6.91) | −11.99 | (6.49) |
| LS Mean (SE) ^{a} | −6.60 | (1.38) | −5.59 | (1.21) | −8.64 | (1.44) | −10.62 | (1.34) |
| LS Mean Diff vs. placebo (95% CI) ^{a} | | | 1.01 | (−2.59, 4.61) | −2.04 | (−5.95, 1.86) | −4.02 | (−7.78, −0.25) |

| | | | |
|---|---|---|---|
| P-value vs. placebo ^{a} | 0.5815 | 0.3055 | 0.0365 |
| Overall p-value for interaction ^{b} | | | 0.9187 |

Baseline ISS7 score

| <13 | | | | | | | | |
|---|---|---|---|---|---|---|---|---|
| Number (observed/imputed) | 10 | (10/0) | 2 | (2/0) | 6 | (5/1) | 2 | (2/0) |
| Mean (SD) | −3.42 | (6.10) | −4.42 | (0.82) | −5.28 | (5.01) | −3.50 | (0.71) |
| LS Mean (SE) ^{a} | −2.72 | (1.55) | −1.87 | (2.70) | −5.14 | (1.96) | −3.73 | (3.33) |
| LS Mean Diff vs. placebo (95% CI) ^{a} | | | 0.85 | (−5.28, 6.98) | −2.42 | (−7.37, 2.52) | −1.01 | (−8.29, 6.27) |
| P-value vs. placebo ^{a} | | | 0.7852 | | 0.3369 | | 0.7855 | |
| ≥13 | | | | | | | | |
| Number (observed/imputed) | 22 | (21/1) | 28 | (25/3) | 20 | (19/1) | 26 | (25/1) |
| Mean (SD) | −5.88 | (6.95) | −5.78 | (4.87) | −8.03 | (6.38) | −10.79 | (5.93) |
| LS Mean (SE) ^{a} | −5.74 | (1.24) | −5.38 | (1.08) | −7.87 | (1.19) | −10.18 | (1.09) |
| LS Mean Diff vs. placebo (95% CI) ^{a} | | | 0.36 | (−2.88, 3.59) | −2.14 | (−5.49, 1.22) | −4.45 | (−7.66, −1.23) |

| | | | |
|---|---|---|---|
| P-value vs. placebo ^{a} | 0.8293 | 0.2124 | 0.0068 |
| Overall p-value for interaction ^{b} | | | 0.5628 |

Duration of disease (years)

| <2 | | | | | | | | |
|---|---|---|---|---|---|---|---|---|
| Number (observed/imputed) | 14 | (13/1) | 10 | (9/1) | 14 | (13/1) | 12 | (11/1) |
| Mean (SD) | −3.63 | (5.63) | −5.46 | (4.49) | −7.53 | (5.34) | −10.93 | (5.46) |
| LS Mean (SE) ^{a} | −3.65 | (1.54) | −4.83 | (1.76) | −8.54 | (1.41) | −9.77 | (1.60) |
| LS Mean Diff vs. placebo (95% CI) ^{a} | | | −1.18 | (−5.77, 3.41) | −4.89 | (−8.99, −0.80) | −6.12 | (−10.48, −1.75) |
| P-value vs. placebo ^{a} | | | 0.6138 | | 0.0191 | | 0.0060 | |
| 2-10 | | | | | | | | |
| Number (observed/imputed) | 15 | (15/0) | 16 | (16/0) | 11 | (10/1) | 12 | (12/0) |
| Mean (SD) | −7.08 | (7.11) | −6.43 | (5.07) | −8.05 | (6.91) | −9.37 | (5.98) |
| LS Mean (SE) ^{a} | −7.03 | (1.42) | −5.24 | (1.27) | −6.89 | (1.50) | −8.84 | (1.51) |
| LS Mean Diff vs. placebo (95% CI) ^{a} | | | 1.79 | (−1.98, 5.56) | 0.14 | (−3.91, 4.18) | −1.82 | (−5.85, 2.22) |
| P-value vs. placebo ^{a} | | | 0.3512 | | 0.9476 | | 0.3780 | |
| >10 | | | | | | | | |
| Number (observed/imputed) | 3 | (3/0) | 4 | (2/2) | 1 | (1/0) | 4 | (4/0) |
| Mean (SD) | −2.17 | (8.81) | −3.29 | (3.90) | 1.67 | (NC) | −11.00 | (8.91) |
| LS Mean (SE) ^{a} | −3.68 | (3.26) | −0.20 | (2.96) | 5.36 | (5.71) | −9.33 | (2.48) |
| LS Mean Diff vs. placebo (95% CI) ^{a} | | | 3.49 | (−5.35, 12.33) | 9.04 | (−4.01, 22.09) | −5.64 | (−13.66, 2.38) |

| | | | |
|---|---|---|---|
| P-value vs. placebo ^{a} | 0.4391 | 0.1743 | 0.1678 |
| Overall p-value for interaction ^{b} | | | 0.4674 |

Baseline H1-antihistamines dose

| 1-fold | | | | | | | | |
|---|---|---|---|---|---|---|---|---|
| Number (observed/imputed) | 17 | (17/0) | 16 | (14/2) | 11 | (10/1) | 18 | (17/1) |
| Mean (SD) | −6.94 | (6.90) | −5.09 | (4.87) | −9.03 | (7.91) | −8.88 | (5.91) |
| LS Mean (SE) ^{a} | −6.83 | (1.42) | −4.24 | (1.40) | −8.44 | (1.60) | −8.05 | (1.33) |
| LS Mean Diff vs. placebo (95% CI) ^{a} | | | 2.59 | (−1.31, 6.49) | −1.60 | (−5.81, 2.60) | −1.22 | (−5.04, 2.59) |
| P-value vs. placebo ^{a} | | | 0.1928 | | 0.4541 | | 0.5295 | |
| 2 to 4-fold standard dose | | | | | | | | |
| Number (observed/imputed) | 14 | (13/1) | 14 | (13/1) | 15 | (14/1) | 10 | (10/0) |
| Mean (SD) | −2.96 | (6.24) | −6.37 | (4.61) | −6.20 | (4.30) | −12.76 | (5.65) |
| LS Mean (SE) ^{a} | −3.48 | (1.49) | −5.28 | (1.43) | −6.73 | (1.31) | −11.00 | (1.62) |
| LS Mean Diff vs. placebo (95% CI) ^{a} | | | −1.80 | (−5.90, 2.30) | −3.25 | (−7.13, 0.62) | −7.52 | (−11.82, −3.22) |

| | | | |
|---|---|---|---|
| P-value vs. placebo ^{a} | 0.3893 | 0.1001 | 0.0006 |
| Overall p-value for interaction ^{b} | | | 0.1602 |

Baseline Eosinophils (10\hat{ }9/L)

| <0.150 | | | | | | | |
|---|---|---|---|---|---|---|---|
| Number | 16 | | N/A | 11 | | 14 | |
| Mean (SD) | N/A | | N/A | N/A | | N/A | |
| LS Mean (SE) | −6.78 | (1.51) | N/A | −6.93 | (1.69) | −10.28 | (1.64) |
| LS Mean Diff vs. placebo (95% CI) | | | N/A | −0.15 | (−4.60, 4.29) | −3.50 | (−7.87, 0.87) |
| P-value vs. placebo | | | N/A | N/A | | N/A | |
| ≥0.150 | | | | | | | |
| Number | 16 | | N/A | 15 | | 14 | |
| Mean (SD) | N/A | | N/A | N/A | | N/A | |
| LS Mean (SE) | −3.99 | (1.42) | N/A | −7.91 | (1.29) | −8.30 | (1.31) |

| | | | | | |
|---|---|---|---|---|---|
| LS Mean Diff vs. placebo (95% CI) | N/A | −3.92 | (−7.74, −0.10) | −4.31 | (−8.09, −0.53) |

| | | | |
|---|---|---|---|
| P-value vs. placebo | N/A | N/A | N/A |

Baseline Eosinophils (10\hat{ }9/L)

| <0.165 (Median) | | | | | | | | |
|---|---|---|---|---|---|---|---|---|
| Number (observed/imputed) | 17 | (17/0) | 16 | (14/2) | 13 | (12/1) | 14 | (14/0) |
| Mean (SD) | −6.64 | (7.14) | −4.93 | (4.13) | −5.92 | (5.31) | −10.99 | (6.77) |
| LS Mean (SE) ^{a} | −6.51 | (1.42) | −4.00 | (1.41) | −7.37 | (1.49) | −10.22 | (1.54) |
| LS Mean Diff vs. placebo (95% CI) ^{a} | | | 2.51 | (−1.40, 6.42) | −0.86 | (−4.88, 3.15) | −3.71 | (−7.81, 0.40) |
| P-value vs. placebo ^{a} | | | 0.2080 | | 0.6742 | | 0.0767 | |
| ≥0.165 (Median) | | | | | | | | |
| Number (observed/imputed) | 15 | (14/1) | 14 | (13/1) | 13 | (12/1) | 14 | (13/1) |
| Mean (SD) | −3.38 | (5.91) | 6.55 | (5.33) | −8.88 | (6.71) | −9.55 | (5.32) |
| LS Mean (SE) ^{a} | −4.08 | (1.51) | −5.54 | (1.44) | −7.62 | (1.45) | −8.24 | (1.39) |
| LS Mean Diff vs. placebo (95% CI) ^{a} | | | −1.46 | (−5.64, 2.72) | −3.53 | (−7.69, 0.63) | −4.16 | (−8.20, −0.11) |

| | | | |
|---|---|---|---|
| P-value vs. placebo ^{a} | 0.4941 | 0.0959 | 0.0440 |
| Overall p-value for interaction ^{b} | | | 0.4740 |

Baseline Eosinophils (10\hat{ }9/L)

| <0.3 | | | | | | | | |
|---|---|---|---|---|---|---|---|---|
| Number (observed/imputed) | 29 | (28/1) | 25 | (22/3) | 20 | (19/1) | 23 | (22/1) |
| Mean (SD) | −4.98 | (7.03) | −5.66 | (4.16) | −7.62 | (6.27) | −10.61 | (6.31) |
| LS Mean (SE) ^{a} | −5.46 | (1.09) | −4.64 | (1.10) | −7.78 | (1.19) | −9.46 | (1.18) |
| LS Mean Diff vs. placebo (95% CI) ^{a} | | | 0.83 | (−2.24, 3.89) | −2.31 | (−5.47, 0.85) | −3.99 | (−7.15, − |
| P-value vs. placebo ^{a} | | | 0.5966 | | 0.1514 | | 0.0132 | |
| ≥0.3 | | | | | | | | |
| Number (observed/imputed) | 3 | (3/0) | 5 | (5/0) | 6 | (5/1) | 5 | (5/0) |
| Mean (SD) | −6.33 | (0.58) | −5.81 | (7.57) | −6.67 | (6.06) | −8.68 | (4.62) |
| LS Mean (SE) ^{a} | −4.85 | (2.85) | −5.53 | (2.57) | −6.15 | (2.12) | −8.12 | (2.13) |
| LS Mean Diff vs. placebo (95% CI) ^{a} | | | −0.68 | (−8.12, 6.76) | −1.29 | (−8.43, 5.85) | −3.27 | (−10.21, 3.67) |

| | | | |
|---|---|---|---|
| P-value vs. placebo ^{a} | 0.8580 | 0.7219 | 0.3547 |
| Overall p-value for interaction ^{b} | | | 0.9753 |

Baseline Total IgE (IU/mL)

| <300 | | | | | | | | |
|---|---|---|---|---|---|---|---|---|
| Number (observed/imputed) | 23 | (22/1) | 21 | (18/3) | 18 | (16/2) | 18 | (18/0) |
| Mean (SD) | −4.45 | (6.94) | −5.41 | (4.78) | −7.93 | (6.60) | −11.12 | (6.60) |
| LS Mean (SE) ^{a} | −4.51 | (1.21) | −4.46 | (1.23) | −7.94 | (1.28) | −9.55 | (1.28) |
| LS Mean Diff vs. placebo (95% CI) ^{a} | | | 0.05 | (−3.34, 3.44) | −3.43 | (−6.90, 0.04) | 5.04 | (−8.49, 1.58) |
| P-value vs. placebo ^{a} | | | 0.9766 | | 0.0524 | | 0.0043 | |
| ≥300 | | | | | | | | |
| Number (observed/imputed) | 9 | (9/0) | 8 | (8/0) | 8 | (8/0) | 10 | (9/1) |
| Mean (SD) | −6.80 | (6.07) | −6.11 | (5.05) | −6.20 | (5.04) | 8.74 | (4.73) |
| LS Mean (SE) ^{a} | −6.54 | (1.94) | −4.90 | (1.83) | −7.17 | (1.88) | −8.12 | (1.78) |
| LS Mean Diff vs. placebo (95% CI) ^{a} | | | 1.64 | (−3.67, 6.95) | −0.62 | (−6.16, 4.91) | −1.58 | (−6.77, 3.62) |

| | | | |
|---|---|---|---|
| P-value vs. placebo ^{a} | 0.5443 | 0.8249 | 0.5523 |
| Overall p-value for interaction ^{b} | | | 0.5546 |

Baseline Total IgE (IU/mL)

| <100 | | | | | | | | |
|---|---|---|---|---|---|---|---|---|
| Number (observed/imputed) | 15 | (14/1) | 11 | (10/1) | 11 | (10/1) | 13 | (13/0) |
| Mean (SD) | −4.06 | (7.77) | −4.48 | (4.37) | −8.52 | (6.05) | −11.17 | (7.09) |
| LS Mean (SE) ^{a} | −4.24 | (1.58) | −3.37 | (1.66) | −8.29 | (1.67) | −9.19 | (1.55) |
| LS Mean Diff vs. placebo (95% CI) ^{a} | | | 0.87 | (−3.65, 5.38) | −4.06 | (−8.57, 0.46) | −4.95 | (−9.29, −0.61) |
| P-value vs. placebo ^{a} | | | 0.7062 | | 0.0780 | | 0.0253 | |
| ≥100 | | | | | | | | |
| Number (observed/imputed) | 17 | (17/0) | 18 | (16/2) | 15 | (14/1) | 15 | (14/1) |
| Mean (SD) | −6.04 | (5.66) | −6.28 | (5.01) | −6.57 | (6.24) | −9.49 | (5.03) |
| LS Mean (SE) ^{a} | −6.03 | (1.33) | −5.51 | (1.28) | −6.91 | (1.31) | −9.13 | (1.38) |
| LS Mean Diff vs. placebo (95% CI) ^{a} | | | 0.52 | (−3.13, 4.18) | −0.88 | (−4.57, 2.80) | −3.10 | (−6.85, 0.65) |

| | | | |
|---|---|---|---|
| P-value vs. placebo ^{a} | 0.7797 | 0.6380 | 0.1053 |
| Overall p-value for interaction ^{b} | | | 0.6170 |

Baseline BHRA

| BHRA+ | | | | | | | | |
|---|---|---|---|---|---|---|---|---|
| Number (observed/imputed) | 3 | (3/0) | 3 | (2/1) | 6 | (5/1) | 4 | (4/0) |
| Mean (SD) | −4.39 | (5.43) | −9.13 | (3.87) | −9.57 | (4.88) | −12.42 | (9.12) |
| LS Mean (SE) ^{a} | −5.38 | (3.35) | −8.50 | (3.41) | −8.42 | (3.14) | −12.96 | (3.21) |
| LS Mean Diff vs. placebo (95% CI) ^{a} | | | −3.12 | (−12.22, 5.98) | −3.03 | (−12.35, 6.28) | −7.58 | (−16.53, 1.37) |
| P-value vs. placebo ^{a} | | | 0.5015 | | 0.5234 | | 0.0967 | |
| BHRA− | | | | | | | | |
| Number (observed/imputed) | 29 | (28/1) | 26 | (24/2) | 20 | (19/1) | 24 | (23/1) |
| Mean (SD) | −5.18 | (6.89) | −5.45 | (4.73) | −6.75 | (6.40) | −9.91 | (5.54) |
| LS Mean (SE) ^{a} | −5.49 | (1.04) | −4.66 | (1.06) | −6.59 | (1.13) | −8.82 | (1.08) |
| LS Mean Diff vs. placebo (95% CI) ^{a} | | | 0.83 | (−2.10, 3.77) | −1.10 | (−4.12, 1.91) | −3.33 | (−6.25, −0.41) |

| | | | |
|---|---|---|---|
| P-value vs. placebo ^{a} | 0.5791 | 0.4730 | 0.0256 |
| Overall p-value for interaction ^{b} | | | 0.5253 |

^{a} Each of the imputed complete data were analyzed by fitting an ANCOVA model with the corresponding baseline value and intervention group as covariates. Analysis was based on the same imputed dataset using WOCF/MI from primary analysis of the primary endpoint.
^{b} Each of the imputed complete data were analyzed by fitting an ANCOVA model with the corresponding baseline value and intervention group as covariates, plus the subgroup variable and the subgroup-by-intervention interaction. Analysis was based on the same imputed dataset using WOCF/MI from primary analysis of the primary endpoint.
^{c} Asia: South Korea, Taiwan, Japan; Latin America: Argentina, Chile; Eastern Europe: Poland; Western Countries: Spain, Italy, Germany, Canada, Greece, Netherlands.
Note:
Descriptive statistics include participants with WOCF imputation at Week 12 and participants whose Week 12 values were imputed by MI were excluded from the descriptive analysis.

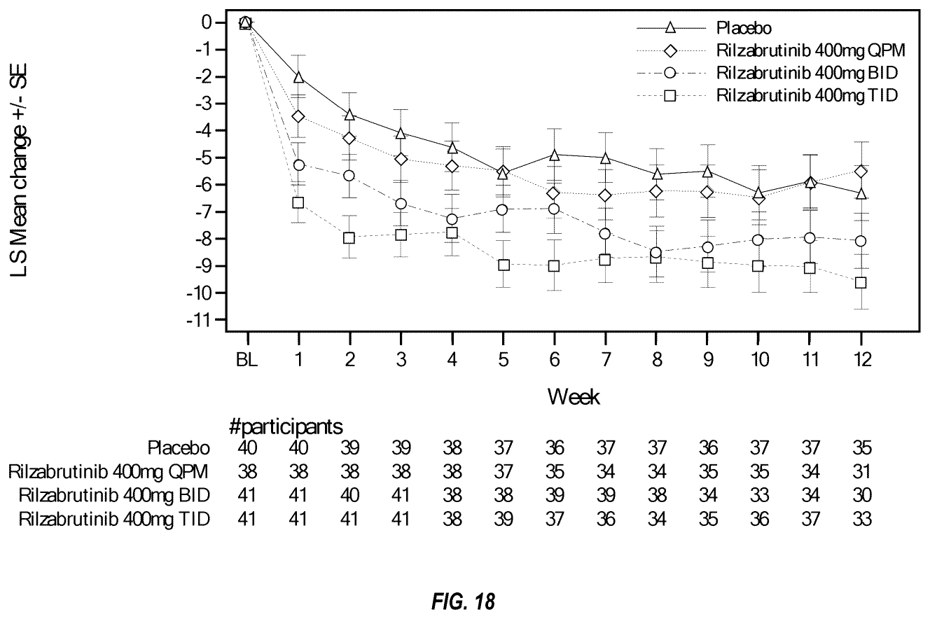

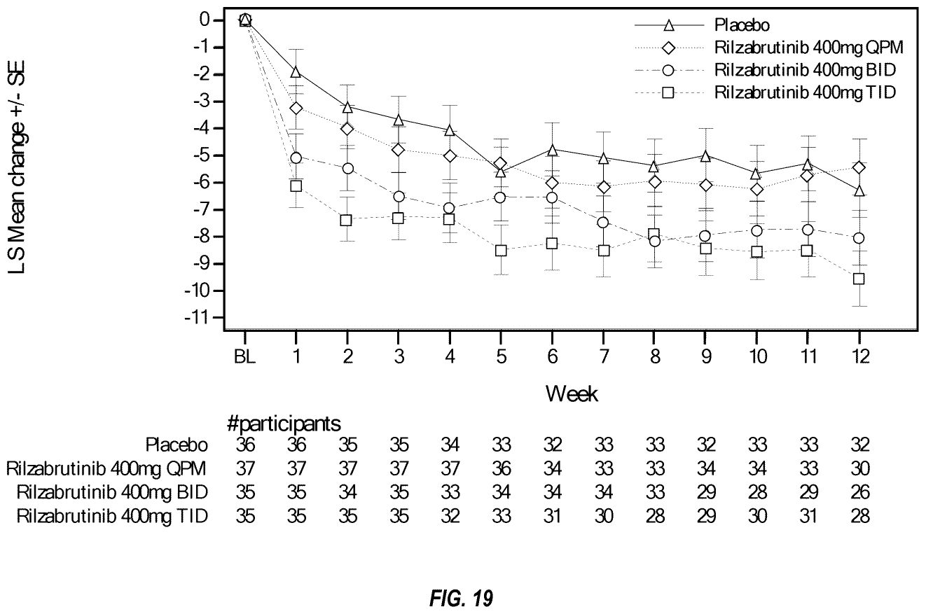

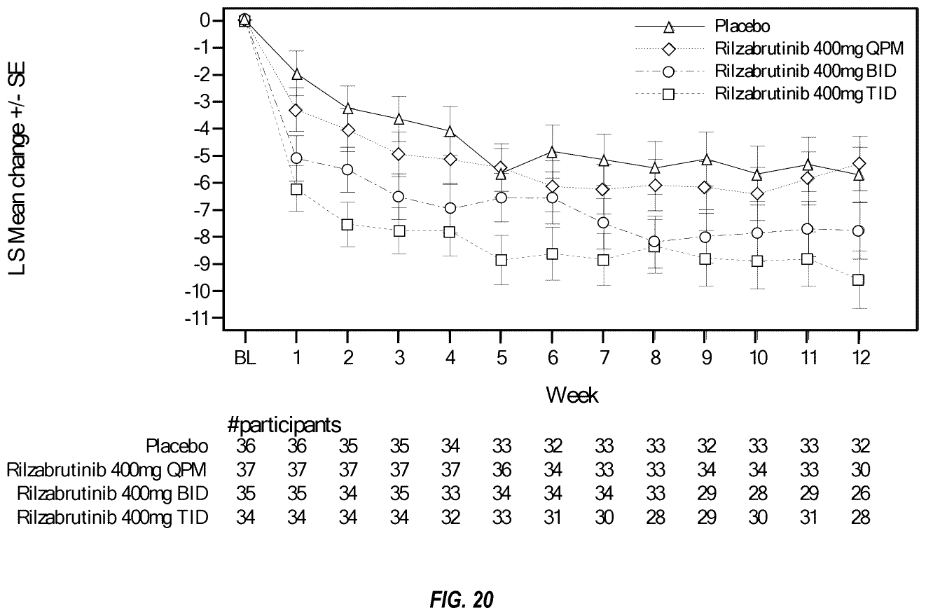

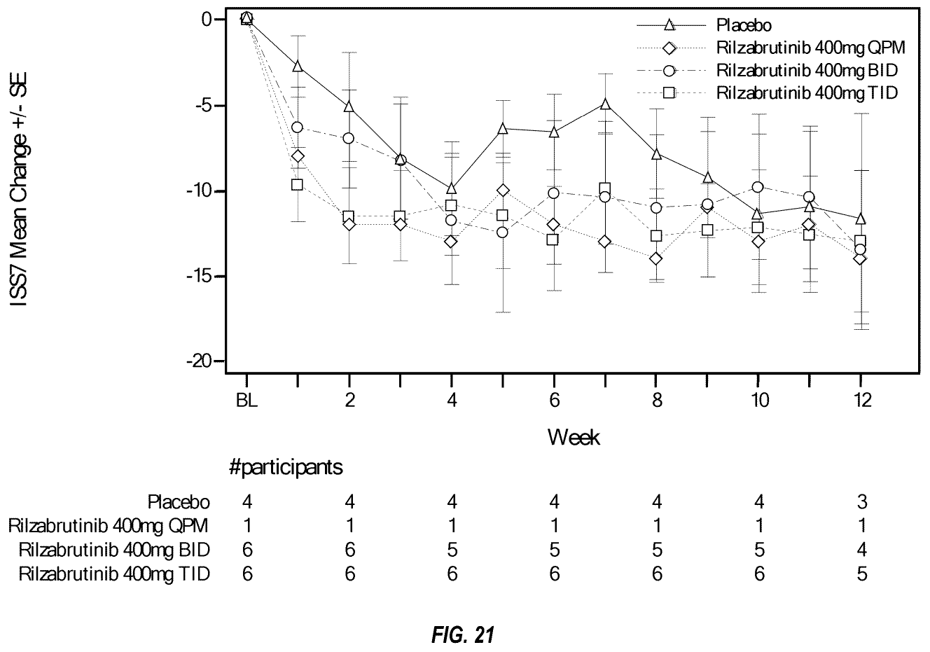

Additionally, participants exhibited a rapid, clinically meaningful improvement in symptoms, with significant reductions in ISS7 observed following one week of treatment (see FIGS. 18-21 and Tables 28-31). In the ITT population, participants receiving 400 mg BID rilzabrutinib had an LS mean change in ISS7 of −5.23 points from baseline (p=0.0024; see Table 29). Consistent with rilzabrutinib's dose-dependent effect on ISS7, participants receiving 400 mg TID showed an LS mean change in ISS7 of −6.65 points (p<0.0001), reflecting a 40.41% decrease from baseline. Notably, the overall reduction in ISS7 observed in both the 400 mg BID and TID arms exceeded the minimal clinically important difference (MCID) of 5 points.

Similar results were observed for the omalizumab-naïve population. Omalizumab-naïve participants in the 400 mg BID and TID rilzabrutinib arms experienced an LS mean reduction in ISS7 of −5.06 (p=0.0060) and −6.13 (p=0.0003), respectively (see Table 30). Similar data were obtained when these analyses were repeated following the removal of the asymptomatic outlier (see Table 31).

As further demonstrated in Tables 28-31, these improvements were sustained over the course of the study.

TABLE 28
Summary of ISS7 by Week in the Double-Blind Period (Omalizumab-Naïve Population and Patients with Prior
Omalizumab Use, Including Omalizumab-Incomplete Responders)

| | | | | | Patients with Prior Omalizumab Use, Including Omalizumab- | | | |
|---|---|---|---|---|---|---|---|---|
| | Omalizumab-Naïve Population | | | | Incomplete Responders | | | |
| | | Rilzabrutinib | Rilzabrutinib | Rilzabrutinib | | Rilzabrutinib | Rilzabrutinib | Rilzabrutinib |
| | Placebo | 400 mg QPM | 400 mg BID | 400 mg TID | Placebo | 400 mg QPM | 400 mg BID | 400 mg TID |
| ISS7 | (N = 36) | (N = 37) | (N = 35) | (N = 35) | (N = 4) | (N = 1) | (N = 6) | (N = 6) |
| Baseline | | | | | | | | |
| Number | 36 | 37 | 35 | 35 | 4 | 1 | 6 | 6 |
| Mean (SD) | 15.14 (4.16) | 16.38 (3.50) | 15.45 (3.72) | 15.69 (4.26) | 19.75 (2.50) | 21.00 (NC) | 17.83 (2.93) | 17.00 (5.25) |
| Median | 14.00 | 16.00 | 14.00 | 14.00 | 21.00 | 21.00 | 18.00 | 19.00 |
| Q1; Q3 | 12.00; 19.50 | 14.00; 20.00 | 13.00; 19.00 | 14.00; 19.00 | 18.50; 21.00 | 21.00; 21.00 | 15.00; 21.00 | 14.00; 21.00 |
| Min; Max | 8.0; 21.0 | 9.0; 21.0 | 8.0; 21.0 | 0.0; 21.0 | 16.0; 21.0 | 21.0; 21.0 | 14.0; 21.0 | 8.0; 21.0 |
| Week 1 | | | | | | | | |
| Number (observed/imputed) | 36 (36/0) | 37 (37/0) | 35 (35/0) | 35 (35/0) | 4 (4/0) | 1 (1/0) | 6 (5/1) | 6 (6/0) |
| Mean (SD) | 13.57 (5.31) | 13.08 (4.52) | 10.51 (5.88) | 9.51 (5.64) | 17.00 (4.08) | 13.00 (NC) | 11.50 (6.19) | 7.33 (5.43) |
| Median | 14.00 | 14.00 | 12.00 | 9.00 | 17.00 | 13.00 | 13.00 | 7.00 |
| Q1; Q3 | 11.00; 17.25 | 11.00; 16.00 | 6.00; 14.00 | 4.00; 15.00 | 13.50; 20.50 | 13.00; 13.00 | 8.00; 16.00 | 2.00; 10.00 |
| Min; Max | 0.0; 21.0 | 2.0; 21.0 | 0.0; 21.0 | 0.0; 21.0 | 13.0; 21.0 | 13.0; 13.0 | 1.0; 18.0 | 2.0; 16.0 |
| Change from baseline | | | | | | | | |
| Number (observed/imputed) | 36 (36/0) | 37 (37/0) | 35 (35/0) | 35 (35/0) | 4 (4/0) | 1 (1/0) | 6 (5/1) | 6 (6/0) |
| Mean (SD) | −1.57 (4.47) | −3.30 (4.93) | −4.94 (4.65) | −6.17 (5.49) | −2.75 (3.59) | −8.00 (NC) | −6.33 (5.82) | −9.67 (5.32) |
| Median | 0.00 | −3.00 | −4.00 | −5.00 | −1.50 | −8.00 | −4.50 | −9.00 |
| Q1; Q3 | −3.75; 1.00 | −5.00; 0.00 | −10.00; −1.00 | −11.00; −2.00 | −5.00; −0.50 | −8.00; −8.00 | −13.00; −2.00 | −15.00; −5.00 |
| Min; Max | −15.0; 8.0 | −17.0; 6.0 | −15.0; 2.0 | −18.0; 3.0 | −8.0; 0.0 | −8.0; −8.0 | −14.0; 0.0 | −16.0; −4.0 |
| Percent change from | | | | | | | | |
| baseline | | | | | | | | |
| Number (observed/imputed) | 36 (36/0) | 37 (37/0) | 35 (35/0) | 34 (34/0) | 4 (4/0) | 1 (1/0) | 6 (5/1) | 6 (6/0) |
| Mean (SD) | −8.95 (33.13) | −17.96 (28.66) | −33.91 (34.62) | −38.58 (33.51) | −13.84 (16.97) | −38.10 (NC) | −35.93 (34.83) | −58.16 (26.72) |
| Median | 0.00 | −19.05 | −25.00 | −31.37 | −8.63 | −38.10 | −23.02 | −66.07 |
| Q1; Q3 | −25.66; 7.42 | −31.25; 0.00 | −57.14; −7.14 | −72.22; −16.67 | −25.30; −2.38 | −38.10; −38.10 | −61.90; −14.29 | −76.19; −28.57 |
| Min; Max | −100.0; 66.7 | −89.5; 40.0 | −100.0; 15.4 | −92.9; 21.4 | −38.1; 0.0 | −38.1; −38.1 | −93.3; 0.0 | −88.2; −23.8 |
| Week 2 | | | | | | | | |
| Number (observed/imputed) | 35 (35/0) | 37 (37/0) | 34 (34/0) | 35 (35/0) | 4 (4/0) | 1 (1/0) | 6 (5/1) | 6 (6/0) |
| Mean (SD) | 12.38 (5.17) | 12.53 (4.73) | 10.32 (5.64) | 8.40 (5.82) | 14.63 (6.94) | 9.00 (NC) | 10.83 (6.85) | 5.50 (6.95) |
| Median | 13.00 | 12.00 | 11.00 | 8.00 | 15.25 | 9.00 | 13.50 | 4.00 |
| Q1; Q3 | 9.00; 16.00 | 10.00; 15.75 | 7.00; 14.00 | 3.00; 11.20 | 8.75; 20.50 | 9.00; 9.00 | 5.00; 15.00 | 0.00; 7.00 |
| Min; Max | 0.0; 21.0 | 3.0; 21.0 | 0.0; 21.0 | 0.0; 21.0 | 7.0; 21.0 | 9.0; 9.0 | 0.0; 18.0 | 0.0; 18.0 |
| Change from baseline | | | | | | | | |
| Number (observed/imputed) | 35 (35/0) | 37 (37/0) | 34 (34/0) | 35 (35/0) | 4 (4/0) | 1 (1/0) | 6 (5/1) | 6 (6/0) |
| Mean (SD) | −2.59 (4.45) | −3.84 (4.75) | −5.00 (4.40) | −7.29 (6.09) | −5.13 (6.38) | −12.00 (NC) | −7.00 (7.04) | −11.50 (6.92) |
| Median | −2.00 | −3.00 | −5.00 | −7.00 | −3.25 | −12.00 | −5.50 | −10.50 |
| Q1; Q3 | −5.00; 0.00 | −6.00; −0.40 | −8.00; −1.00 | −13.00; −2.80 | −9.75; −0.50 | −12.00; −12.00 | −15.00; 0.00 | −17.00; −7.00 |
| Min; Max | −15.0; 6.0 | −16.0; 4.7 | −16.0; 1.0 | −17.0; 4.7 | −14.0; 0.0 | −12.0; −12.0 | −16.0; 0.0 | −21.0; −3.0 |
| Percent change from | | | | | | | | |
| baseline | | | | | | | | |
| Number (observed/imputed) | 35 (35/0) | 37 (37/0) | 34 (34/0) | 34 (34/0) | 4 (4/0) | 1 (1/0) | 6 (5/1) | 6 (6/0) |
| Mean (SD) | −16.14 (31.66) | −22.08 (27.52) | −34.40 (31.27) | −46.12 (36.57) | −26.45 (30.82) | −57.14 (NC) | −38.76 (40.94) | −69.74 (33.53) |
| Median | −12.50 | −17.11 | −25.06 | −46.89 | −19.57 | −57.14 | −28.17 | −77.08 |
| Q1; Q3 | −28.57; 0.00 | −35.71; −2.00 | −53.33; −7.14 | −66.67; −21.43 | −50.52; −2.38 | −57.14; −57.14 | −76.19; 0.00 | −100.00; −50.00 |
| Min; Max | −100.0; 50.0 | −84.2; 33.3 | −100.0; 7.7 | −100.0; 33.3 | −66.7; 0.0 | −57.1; −57.1 | −100.0; 0.0 | −100.0; −14.3 |
| Week 3 | | | | | | | | |
| Number (observed/imputed) | 35 (35/0) | 37 (37/0) | 35 (35/0) | 35 (35/0) | 4 (4/0) | 1 (1/0) | 6 (5/1) | 6 (6/0) |
| Mean (SD) | 11.98 (5.40) | 11.60 (4.68) | 9.16 (6.09) | 8.34 (5.42) | 11.63 (6.46) | 9.00 (NC) | 9.53 (7.26) | 5.50 (5.50) |
| Median | 13.00 | 11.00 | 9.00 | 8.40 | 12.43 | 9.00 | 10.50 | 5.00 |
| Q1; Q3 | 10.00; 15.17 | 8.00; 15.40 | 4.00; 13.00 | 3.00; 12.83 | 7.63; 15.63 | 9.00; 9.00 | 3.00; 15.17 | 1.00; 7.00 |
| Min; Max | 0.0; 21.0 | 3.0; 21.0 | 0.0; 21.0 | 0.0; 21.0 | 3.0; 18.7 | 9.0; 9.0 | 0.0; 18.0 | 0.0; 15.0 |
| Change from baseline | | | | | | | | |
| Number (observed/imputed) | 35 (35/0) | 37 (37/0) | 35 (35/0) | 35 (35/0) | 4 (4/0) | 1 (1/0) | 6 (5/1) | 6 (6/0) |
| Mean (SD) | −2.99 (4.61) | −4.77 (4.84) | −6.29 (5.74) | −7.34 (6.48) | −8.12 (7.16) | −12.00 (NC) | −8.31 (8.28) | −11.50 (6.47) |
| Median | −2.00 | −3.67 | 5.00 | −7.00 | −6.08 | −12.00 | −9.00 | −10.50 |
| Q1; Q3 | −7.00; 0.00 | −8.00; −1.00 | −11.00; −1.00 | −12.00; −3.00 | −13.38; −2.87 | −12.00; −12.00 | −12.00; 0.00 | −16.00; −6.00 |
| Min; Max | −16.0; 4.0 | −16.0; 2.0 | −19.0; 0.0 | −17.0; 14.0 | −18.0; −2.3 | −12.0; −12.0 | −21.0; 1.2 | −21.0; −5.0 |
| Percent change from | | | | | | | | |
| baseline | | | | | | | | |
| Number (observed/imputed) | 35 (35/0) | 37 (37/0) | 35 (35/0) | 34 (34/0) | 4 (4/0) | 1 (1/0) | 6 (5/1) | 6 (6/0) |
| Mean (SD) | −18.75 (33.73) | −27.79 (26.72) | −40.77 (35.75) | −49.02 (33.06) | −39.94 (33.06) | −57.14 (NC) | −44.35 (43.62) | −66.98 (26.87) |
| Median | −9.52 | −23.81 | −26.67 | −48.53 | −31.46 | −57.14 | −47.22 | −64.58 |
| Q1; Q3 | −33.33; 0.00 | −47.62; −8.33 | −73.33; −11.11 | −78.57; −22.22 | 63.69; −16.18 | −57.14; −57.14 | −80.00; 0.00 | −94.12; −50.00 |
| Min; Max | −100.0; 33.3 | −84.2; 13.3 | −100.0; 0.0 | −100.0; 23.1 | −85.7; −11.1 | −57.1; −57.1 | −100.0; 8.3 | −100.0; −28.6 |
| Week 4 | | | | | | | | |
| Number (observed/imputed) | 34 (34/0) | 37 (36/1) | 33 (33/0) | 32 (32/0) | 4 (4/0) | 1 (1/0) | 5 (4/1) | 6 (6/0) |
| Mean (SD) | 11.51 (6.14) | 11.39 (4.79) | 8.32 (6.02) | 7.79 (5.03) | 9.84 (5.24) | 8.00 (NC) | 6.80 (8.53) | 6.17 (6.41) |
| Median | 12.33 | 11.00 | 7.00 | 7.50 | 10.68 | 8.00 | 1.00 | 5.42 |
| Q1; Q3 | 7.00; 15.00 | 8.00; 15.00 | 3.00; 13.00 | 4.33; 11.00 | 5.88; 13.80 | 8.00; 8.00 | 1.00; 14.00 | 1.17; 7.00 |
| Min; Max | 0.0; 21.0 | 1.2; 21.0 | 0.0; 21.0 | 0.0; 21.0 | 3.0; 15.0 | 8.0; 8.0 | 0.0; 18.0 | 0.0; 18.0 |
| Change from baseline | | | | | | | | |
| Number (observed/imputed) | 34 (34/0) | 37 (36/1) | 33 (33/0) | 32 (32/0) | 4 (4/0) | 1 (1/0) | 5 (4/1) | 6 (6/0) |
| Mean (SD) | −3.54 (5.46) | −4.99 (5.13) | −6.94 (6.13) | −8.30 (5.13) | −9.91 (5.48) | −13.00 (NC) | −11.80 (8.35) | −10.83 (7.32) |
| Median | −2.42 | −4.00 | −5.83 | −8.00 | −7.83 | −13.00 | −14.00 | −11.08 |
| Q1; Q3 | −7.00; 0.00 | −7.00; −1.00 | −11.00; −2.00 | −12.00; −4.30 | −13.20; −6.63 | −13.00; −13.00 | −17.00; −7.00 | −15.83; −3.00 |
| Min; Max | −17.0; 8.0 | −17.8; 1.0 | −19.0; 2.0 | −19.0; 0.0 | −18.0; −6.0 | −13.0; −13.0 | −21.0; 0.0 | −21.0; −3.0 |
| Percent change from | | | | | | | | |
| baseline | | | | | | | | |
| Number (observed/imputed) | 34 (34/0) | 37 (36/1) | 33 (33/0) | 32 (32/0) | 4 (4/0) | 1 (1/0) | 5 (4/1) | 6 (6/0) |
| Mean (SD) | −22.34 (39.94) | −28.81 (27.96) | −44.87 (37.61) | −51.09 (29.31) | −49.90 (24.88) | −61.90 (NC) | −64.22 (45.05) | −61.65 (32.63) |
| Median | −17.71 | −23.81 | −41.67 | −50.00 | −42.66 | −61.90 | −93.33 | −62.50 |
| Q1; Q3 | −50.00; 0.00 | −50.00; −6.67 | −80.00; −15.38 | −72.56; −27.78 | −65.51; −34.29 | −61.90; −61.90 | −94.44; −33.33 | −93.14; −37.50 |
| Min; Max | −100.0; 61.5 | −93.9; 6.7 | −100.0; 22.2 | −100.0; 0.0 | −85.7; −28.6 | −61.9; −61.9 | −100.0; 0.0 | −100.0; −14.3 |
| Week 5 | | | | | | | | |
| Number (observed/imputed) | 33 (33/0) | 36 (35/1) | 34 (33/1) | 33 (33/0) | 4 (4/0) | 1 (1/0) | 4 (3/1) | 6 (5/1) |
| Mean (SD) | 9.75 (4.75) | 11.00 (4.68) | 8.88 (6.81) | 6.92 (5.87) | 13.35 (4.77) | 11.00 (NC) | 5.50 (8.54) | 5.50 (6.53) |
| Median | 9.00 | 9.90 | 8.50 | 7.00 | 13.38 | 11.00 | 2.00 | 3.00 |
| Q1; Q3 | 7.00; 12.83 | 8.00; 14.00 | 3.00; 12.83 | 2.00; 9.00 | 9.50; 17.21 | 11.00; 11.00 | 0.00; 11.00 | 2.00; 7.00 |
| Min; Max | 0.0; 21.0 | 0.0; 21.0 | 0.0; 21.0 | 0.0; 21.0 | 8.0; 18.7 | 11.0; 11.0 | 0.0; 18.0 | 0.0; 18.0 |
| Change from baseline | | | | | | | | |
| Number (observed/imputed) | 33 (33/0) | 36 (35/1) | 34 (33/1) | 33 (33/0) | 4 (4/0) | 1 (1/0) | 4 (3/1) | 6 (5/1) |
| Mean (SD) | −5.35 (4.29) | −5.53 (4.98) | −6.55 (6.17) | −9.18 (6.46) | −6.40 (3.34) | −10.00 (NC) | −12.50 (9.33) | −11.50 (7.58) |
| Median | −5.00 | −4.10 | −6.42 | −9.00 | −6.63 | −10.00 | −14.50 | −10.50 |
| Q1; Q3 | −8.00; −2.00 | −8.50; −1.50 | −11.83; −1.00 | −13.60; −5.00 | −9.00; −3.79 | −10.00; −10.00 | −19.50; −5.50 | −19.00; −5.00 |
| Min; Max | −16.0; 3.0 | −19.0; 1.0 | −17.7; 6.0 | −21.0; 1.4 | −10.0; −2.3 | −10.0; −10.0 | −21.0; 0.0 | −21.0; −3.0 |
| Percent change from | | | | | | | | |
| baseline | | | | | | | | |
| Number (observed/imputed) | 33 (33/0) | 36 (35/1) | 34 (33/1) | 33 (33/0) | 4 (4/0) | 1 (1/0) | 4 (3/1) | 6 (5/1) |
| Mean (SD) | −34.77 (28.93) | −31.93 (26.18) | −43.73 (41.21) | −55.68 (34.88) | −33.43 (18.66) | −47.62 (NC) | −68.33 (47.26) | −66.60 (31.51) |
| Median | −38.10 | −30.00 | −39.85 | −56.25 | −36.31 | −47.62 | −86.67 | −72.43 |
| Q1; Q3 | −50.00; −18.18 | −48.16; −9.11 | −80.00; −7.69 | −85.71; −27.78 | −48.81; −18.06 | −47.62; −47.62 | −100.00; −36.67 | −90.48; −50.00 |
| Min; Max | −100.0; 37.5 | −100.0; 5.0 | −100.0; 40.0 | −100.0; 10.0 | −50.0; −11.1 | −47.6; −47.6 | −100.0; 0.0 | −100.0; −14.3 |
| Week 6 | | | | | | | | |
| Number (observed/imputed) | 32 (31/1) | 34 (33/1) | 34 (33/1) | 31 (31/0) | 4 (3/1) | 1 (1/0) | 5 (4/1) | 6 (5/1) |
| Mean (SD) | 10.58 (5.85) | 10.27 (5.26) | 8.85 (6.40) | 7.32 (6.05) | 13.17 (5.82) | 9.00 (NC) | 7.07 (8.32) | 4.17 (6.88) |
| Median | 11.60 | 10.25 | 10.00 | 7.00 | 12.33 | 9.00 | 2.33 | 2.00 |
| Q1; Q3 | 5.50; 14.00 | 6.00; 14.00 | 2.00; 14.00 | 1.00; 10.50 | 9.33; 17.00 | 9.00; 9.00 | 1.00; 14.00 | 0.00; 3.00 |
| Min; Max | 0.0; 21.0 | 0.0; 21.0 | 0.0; 21.0 | 0.0; 21.0 | 7.0; 21.0 | 9.0; 9.0 | 0.0; 18.0 | 0.0; 18.0 |
| Change from baseline | | | | | | | | |
| Number (observed/imputed) | 32 (31/1) | 34 (33/1) | 34 (33/1) | 31 (31/0) | 4 (3/1) | 1 (1/0) | 5 (4/1) | 6 (5/1) |
| Mean (SD) | −4.54 (5.55) | −6.59 (5.45) | −6.58 (5.90) | −8.71 (6.32) | −6.58 (4.43) | −12.00 (NC) | −10.13 (9.42) | −12.83 (7.49) |
| Median | −4.00 | −5.00 | −5.75 | −7.50 | −8.50 | −12.00 | −14.00 | −14.50 |
| Q1; Q3 | −7.40; −0.25 | −11.00; −3.00 | −12.00; −1.00 | −14.00; −3.33 | −9.17; −4.00 | −12.00; −12.00 | −18.00; 0.00 | −19.00; −5.00 |
| Min; Max | −20.0; 6.0 | −19.0; 1.0 | −18.0; 2.0 | −21.0; 2.3 | −9.3; 0.0 | −12.0; −12.0 | −18.7; 0.0 | −21.0; −3.0 |
| Percent change from | | | | | | | | |
| baseline | | | | | | | | |
| Number (observed/imputed) | 32 (31/1) | 34 (33/1) | 34 (33/1) | 31 (31/0) | 4 (3/1) | 1 (1/0) | 5 (4/1) | 6 (5/1) |
| Mean (SD) | −28.70 (38.25) | −38.20 (29.52) | −42.90 (39.02) | −53.59 (35.59) | −34.70 (24.32) | −57.14 (NC) | −56.44 (51.68) | −75.50 (33.00) |
| Median | −25.00 | −31.67 | −33.40 | −50.00 | −41.27 | −57.14 | −88.89 | −88.10 |
| Q1; Q3 | −57.74; −2.27 | −64.71; −20.00 | −84.62; −6.67 | −95.24; −25.00 | 50.35; −19.05 | −57.14; −57.14 | −93.33; 0.00 | −100.00; −62.50 |
| Min; Max | −100.0; 62.5 | −100.0; 6.7 | −100.0; 22.2 | −100.0; 16.7 | −56.3; 0.0 | −57.1; −57.1 | −100.0; 0.0 | −100.0; −14.3 |
| Week 7 | | | | | | | | |
| Number (observed/imputed) | 33 (32/1) | 33 (31/2) | 34 (33/1) | 30 (30/0) | 4 (3/1) | 1 (1/0) | 5 (4/1) | 6 (5/1) |
| Mean (SD) | 10.51 (5.70) | 10.68 (5.34) | 8.21 (6.16) | 7.14 (5.35) | 14.85 (5.21) | 8.00 (NC) | 6.80 (8.56) | 7.06 (6.25) |
| Median | 9.33 | 9.80 | 7.00 | 8.00 | 15.00 | 8.00 | 2.00 | 6.00 |
| Q1; Q3 | 7.00; 14.00 | 7.00; 14.00 | 2.80; 12.00 | 3.00; 10.00 | 11.20; 18.50 | 8.00; 8.00 | 0.00; 14.00 | 3.00; 9.33 |
| Min; Max | 0.0; 21.0 | 0.0; 21.0 | 0.0; 21.0 | 0.0; 21.0 | 8.4; 21.0 | 8.0; 8.0 | 0.0; 18.0 | 0.0; 18.0 |
| Change from baseline | | | | | | | | |
| Number (observed/imputed) | 33 (32/1) | 33 (31/2) | 34 (33/1) | 30 (30/0) | 4 (3/1) | 1 (1/0) | 5 (4/1) | 6 (5/1) |
| Mean (SD) | −4.58 (5.48) | −6.11 (5.15) | −7.28 (5.95) | −8.96 (5.57) | −4.90 (3.45) | −13.00 (NC) | −10.40 (9.91) | −9.94 (7.89) |
| Median | −4.20 | −5.20 | −8.08 | −9.00 | −6.00 | −13.00 | −13.00 | −7.33 |
| Q1; Q3 | −7.00; −0.83 | 12.00; −1.17 | −12.00; −2.00 | −14.00; −5.25 | −7.30; −2.50 | −13.00; −13.00 | −18.00; 0.00 | −18.00; −3.00 |
| Min; Max | −20.0; 5.0 | −14.0; 1.0 | −19.0; 2.0 | −20.0; 1.8 | −7.6; 0.0 | −13.0; −13.0 | −21.0; 0.0 | −21.0; −3.0 |
| Percent change from | | | | | | | | |
| baseline | | | | | | | | |
| Number (observed/imputed) | 33 (32/1) | 33 (31/2) | 34 (33/1) | 30 (30/0) | 4 (3/1) | 1 (1/0) | 5 (4/1) | 6 (5/1) |
| Mean (SD) | −28.57 (37.46) | −36.22 (29.09) | −46.93 (37.51) | −55.24 (31.64) | −26.16 (19.97) | −61.90 (NC) | −57.33 (52.62) | −54.94 (32.91) |
| Median | −25.76 | −37.78 | −56.35 | −52.66 | −28.57 | −61.90 | −86.67 | −48.16 |
| Q1; Q3 | −50.00; −5.21 | −60.00; −8.33 | −85.71; −14.29 | −84.21; −37.78 | −40.42; −11.90 | −61.90; −61.90 | −100.00; 0.00 | −85.71; −33.33 |
| Min; Max | −100.0; 57.5 | −100.0; 7.7 | −100.0; 22.2 | −100.0; 12.5 | −47.5; 0.0 | −61.9; −61.9 | −100.0; 0.0 | −100.0; −14.3 |
| Week 8 | | | | | | | | |
| Number (observed/imputed) | 33 (32/1) | 33 (30/3) | 33 (32/1) | 28 (28/0) | 4 (3/1) | 1 (1/0) | 5 (4/1) | 6 (5/1) |
| Mean (SD) | 10.15 (6.23) | 10.72 (5.26) | 7.33 (6.55) | 7.24 (5.51) | 11.91 (6.45) | 7.00 (NC) | 6.20 (8.32) | 4.33 (6.83) |
| Median | 9.00 | 10.50 | 6.00 | 7.00 | 10.40 | 7.00 | 1.00 | 1.50 |
| Q1; Q3 | 7.00; 15.00 | 7.00; 14.00 | 2.00; 11.00 | 2.50; 11.60 | 7.82; 16.00 | 7.00; 7.00 | 0.00; 12.00 | 1.00; 4.00 |
| Min; Max | 0.0; 21.0 | 0.0; 21.0 | 0.0; 21.0 | 0.0; 21.0 | 5.8; 21.0 | 7.0; 7.0 | 0.0; 18.0 | 0.0; 18.0 |
| Change from baseline | | | | | | | | |
| Number (observed/imputed) | 33 (32/1) | 33 (30/3) | 33 (32/1) | 28 (28/0) | 4 (3/1) | 1 (1/0) | 5 (4/1) | 6 (5/1) |
| Mean (SD) | −4.94 (6.17) | −6.03 (4.99) | −8.15 (6.15) | −9.01 (5.43) | −7.84 (5.25) | −14.00 (NC) | −11.00 (9.49) | −12.67 (6.71) |
| Median | −5.00 | −6.00 | −9.00 | −9.33 | −10.08 | −14.00 | −14.00 | −14.00 |
| Q1; Q3 | −9.00; −0.33 | −9.17; −2.00 | −12.00; −2.17 | −13.50; −6.00 | −10.68; −5.00 | −14.00; −14.00 | −18.00; −2.00 | −17.00; −7.00 |
| Min; Max | −18.0; 7.0 | −19.0; 1.0 | −21.0; 2.0 | −21.0; 1.0 | −11.2; 0.0 | −14.0; −14.0 | −21.0; 0.0 | −21.0; −3.0 |
| Percent change from | | | | | | | | |
| baseline | | | | | | | | |
| Number (observed/imputed) | 33 (32/1) | 33 (30/3) | 33 (32/1) | 28 (28/0) | 4 (3/1) | 1 (1/0) | 5 (4/1) | 6 (5/1) |
| Mean (SD) | −31.17 (41.33) | −36.14 (28.79) | −53.11 (38.61) | −55.59 (31.84) | −41.12 (28.20) | −66.67 (NC) | −61.52 (49.97) | −77.10 (31.49) |
| Median | −33.33 | −33.68 | −60.00 | −53.49 | −50.48 | −66.67 | −93.33 | −86.61 |
| Q1; Q3 | −50.00; −1.75 | −58.33; −13.33 | −90.00; −19.05 | −81.78; −33.33 | −58.44; −23.81 | −66.67; −66.67 | −100.00; −14.29 | −94.12; −80.95 |
| Min; Max | −100.0; 54.0 | −100.0; 7.7 | −100.0; 22.2 | −100.0; 9.1 | −63.5; 0.0 | −66.7; −66.7 | −100.0; 0.0 | −100.0; −14.3 |
| Week 9 | | | | | | | | |
| Number (observed/imputed) | 32 (31/1) | 34 (31/3) | 29 (28/1) | 29 (29/0) | 4 (3/1) | 1 (1/0) | 5 (4/1) | 6 (5/1) |
| Mean (SD) | 10.55 (5.94) | 10.46 (5.15) | 7.63 (7.11) | 6.68 (5.31) | 10.50 (7.09) | 10.00 (NC) | 6.37 (8.47) | 4.64 (6.72) |
| Median | 10.75 | 10.25 | 7.00 | 7.00 | 7.70 | 10.00 | 1.00 | 2.00 |
| Q1; Q3 | 7.00; 14.00 | 8.00; 14.00 | 1.17; 11.00 | 2.00; 9.00 | 6.30; 14.70 | 10.00; 10.00 | 0.00; 12.83 | 1.17; 4.67 |
| Min; Max | 0.0; 21.0 | 0.0; 21.0 | 0.0; 21.0 | 0.0; 21.0 | 5.6; 21.0 | 10.0; 10.0 | 0.0; 18.0 | 0.0; 18.0 |
| Change from baseline | | | | | | | | |
| Number (observed/imputed) | 32 (31/1) | 34 (31/3) | 29 (28/1) | 29 (29/0) | 4 (3/1) | 1 (1/0) | 5 (4/1) | 6 (5/1) |
| Mean (SD) | −4.58 (5.77) | −6.39 (5.27) | −7.57 (6.14) | −9.70 (5.57) | −9.25 (7.04) | 11.00 (NC) | −10.83 (9.53) | −12.36 (6.72) |
| Median | −4.50 | −5.42 | −8.00 | −10.00 | −10.80 | −11.00 | −15.00 | −13.92 |
| Q1; Q3 | −8.00; 0.00 | −9.00; −2.50 | −11.00; −3.00 | −14.00; −6.00 | −14.70; −3.80 | −11.00; −11.00 | −18.00; −1.17 | −16.33; −6.00 |
| Min; Max | −17.0; 8.0 | −19.0; 1.0 | −20.0; 3.0 | −21.0; 0.7 | −15.4; 0.0 | −11.0; −11.0 | −20.0; 0.0 | −21.0; −3.0 |
| Percent change from | | | | | | | | |
| baseline | | | | | | | | |
| Number (observed/imputed) | 32 (31/1) | 34 (31/3) | 29 (28/1) | 29 (29/0) | 4 (3/1) | 1 (1/0) | 5 (4/1) | 6 (5/1) |
| Mean (SD) | −28.39 (39.68) | −37.00 (28.06) | −51.57 (40.77) | −58.56 (30.66) | −46.88 (33.11) | −52.38 (NC) | −60.71 (51.74) | −74.49 (30.89) |
| Median | −28.33 | −33.33 | −53.33 | −65.00 | −57.08 | −52.38 | −95.24 | −83.01 |
| Q1; Q3 | −55.00; 0.00 | −53.33; −19.23 | −91.67; −16.67 | −85.71; −33.33 | −70.00; −23.75 | −52.38; −52.38 | −100.00; −8.33 | −91.67; −75.00 |
| Min; Max | −100.0; 61.5 | −100.0; 5.0 | −100.0; 23.1 | −100.0; 6.1 | −73.3; 0.0 | −52.4; −52.4 | −100.0; 0.0 | −100.0; −14.3 |
| Week 10 | | | | | | | | |
| Number (observed/imputed) | 33 (32/1) | 34 (31/3) | 28 (27/1) | 30 (30/0) | 4 (3/1) | 1 (1/0) | 5 (4/1) | 6 (4/2) |
| Mean (SD) | 10.00 (6.49) | 10.43 (5.30) | 8.17 (7.29) | 6.75 (5.25) | 8.39 (8.95) | 8.00 (NC) | 7.40 (8.11) | 4.83 (6.85) |
| Median | 9.33 | 9.50 | 7.00 | 7.00 | 5.70 | 8.00 | 4.00 | 3.00 |
| Q1; Q3 | 5.25; 14.00 | 7.00; 14.00 | 0.00; 11.50 | 2.00; 10.00 | 2.08; 14.70 | 8.00; 8.00 | 1.00; 14.00 | 0.00; 5.00 |
| Min; Max | 0.0; 21.0 | 0.0; 21.0 | 0.0; 21.0 | 0.0; 21.0 | 1.2; 21.0 | 8.0; 8.0 | 0.0; 18.0 | 0.0; 18.0 |
| Change from baseline | | | | | | | | |
| Number (observed/imputed) | 33 (32/1) | 34 (31/3) | 28 (27/1) | 30 (30/0) | 4 (3/1) | 1 (1/0) | 5 (4/1) | 6 (4/2) |
| Mean (SD) | −5.09 (5.89) | −6.43 (5.26) | −7.07 (6.58) | −9.55 (5.37) | −11.36 (9.29) | −13.00 (NC) | −9.80 (9.55) | −12.17 (8.26) |
| Median | −5.00 | −6.00 | −7.58 | −10.00 | −12.80 | −13.00 | −11.00 | −13.00 |
| Q1; Q3 | −8.17; 0.00 | −9.00; −3.00 | −12.50; −2.00 | −14.00; −5.00 | −18.92; −3.80 | −13.00; −13.00 | −18.00; 0.00 | −20.00; −3.00 |
| Min; Max | −17.0; 8.0 | −19.0; 2.2 | −20.0; 5.0 | −21.0; 0.0 | −19.8; 0.0 | −13.0; −13.0 | −20.0; 0.0 | −21.0; −3.0 |
| Percent change from | | | | | | | | |
| baseline | | | | | | | | |
| Number (observed/imputed) | 33 (32/1) | 34 (31/3) | 28 (27/1) | 30 (30/0) | 4 (3/1) | 1 (1/0) | 5 (4/1) | 6 (4/2) |
| Mean (SD) | −33.59 (41.95) | −37.25 (29.62) | −47.10 (43.27) | −58.27 (30.87) | −56.91 (43.07) | −61.90 (NC) | −53.71 (50.05) | −68.55 (36.38) |
| Median | −33.33 | −39.23 | −48.10 | −57.19 | −66.61 | −61.90 | −73.33 | −79.76 |
| Q1; Q3 | −55.79; 0.00 | −57.14; −17.11 | −100.00; −13.39 | −84.62; −33.33 | −90.08; −23.75 | −61.90; −61.90 | −95.24; 0.00 | −100.00; −37.50 |
| Min; Max | −100.0; 61.5 | −100.0; 24.4 | −100.0; 38.5 | −100.0; 0.0 | −94.4; 0.0 | −61.9; −61.9 | −100.0; 0.0 | −100.0; −14.3 |
| Week 11 | | | | | | | | |
| Number (observed/imputed) | 33 (32/1) | 33 (30/3) | 29 (28/1) | 31 (31/0) | 4 (3/1) | 1 (1/0) | 5 (4/1) | 6 (4/2) |
| Mean (SD) | 10.24 (6.31) | 11.03 (5.55) | 7.93 (6.94) | 6.99 (5.53) | 8.79 (8.61) | 9.00 (NC) | 6.77 (8.14) | 4.42 (6.99) |
| Median | 11.00 | 11.00 | 7.00 | 7.00 | 6.50 | 9.00 | 2.00 | 1.75 |
| Q1; Q3 | 6.00; 14.00 | 7.00; 14.00 | 3.00; 10.00 | 2.33; 10.00 | 3.08; 14.50 | 9.00; 9.00 | 1.00; 12.83 | 0.00; 5.00 |
| Min; Max | 0.0; 21.0 | 0.0; 21.0 | 0.0; 21.0 | 0.0; 21.0 | 1.2; 21.0 | 9.0; 9.0 | 0.0; 18.0 | 0.0; 18.0 |
| Change from baseline | | | | | | | | |
| Number (observed/imputed) | 33 (32/1) | 33 (30/3) | 29 (28/1) | 31 (31/0) | 4 (3/1) | 1 (1/0) | 5 (4/1) | 6 (4/2) |
| Mean (SD) | −4.85 (6.05) | −6.07 (4.83) | −7.51 (6.18) | −9.23 (5.92) | −10.96 (8.81) | −12.00 (NC) | −10.43 (9.36) | −12.58 (8.36) |
| Median | −6.00 | −6.00 | −8.00 | −9.00 | −12.00 | −12.00 | −13.00 | −13.75 |
| Q1; Q3 | −9.00; 0.00 | −8.00; −3.00 | −12.00; −2.80 | −14.00; −5.20 | −17.92; −4.00 | −12.00; −12.00 | −18.00; −1.17 | −21.00; −3.00 |
| Min; Max | −17.0; 8.0 | −19.0; 1.0 | −18.0; 4.0 | −21.0; 4.2 | −19.8; 0.0 | −12.0; −12.0 | −20.0; 0.0 | −21.0; −3.0 |
| Percent change from | | | | | | | | |
| baseline | | | | | | | | |
| Number (observed/imputed) | 33 (32/1) | 33 (30/3) | 29 (28/1) | 31 (31/0) | 4 (3/1) | 1 (1/0) | 5 (4/1) | 6 (4/2) |
| Mean (SD) | −30.60 (43.70) | −36.12 (28.12) | −49.75 (40.18) | −56.08 (33.60) | −55.16 (41.05) | −57.14 (NC) | −58.05 (49.51) | −71.13 (37.09) |
| Median | −33.33 | −30.00 | −57.14 | −60.00 | −63.10 | −57.14 | −86.67 | −87.50 |
| Q1; Q3 | −56.25; 0.00 | −50.00; −18.33 | −80.95; −25.00 | −85.71; −33.33 | −85.32; −25.00 | −57.14; −57.14 | −95.24; −8.33 | −100.00; −37.50 |
| Min; Max | −100.0; 61.5 | −100.0; 5.0 | −100.0; 23.5 | −100.0; 30.0 | −94.4; 0.0 | −57.1; −57.1 | −100.0; 0.0 | −100.0; −14.3 |
| Week 12 | | | | | | | | |
| Number (observed/imputed) | 32 (31/1) | 30 (27/3) | 26 (24/2) | 28 (27/1) | 3 (2/1) | 1 (1/0) | 4 (3/1) | 5 (3/2) |
| Mean (SD) | 10.02 (6.82) | 11.51 (5.54) | 8.09 (6.95) | 5.77 (5.26) | 9.33 (10.69) | 7.00 (NC) | 4.50 (9.00) | 4.60 (7.80) |
| Median | 8.50 | 11.60 | 7.00 | 7.00 | 7.00 | 7.00 | 0.00 | 0.00 |
| Q1; Q3 | 4.83; 14.00 | 7.00; 15.17 | 1.17; 13.00 | 0.00; 8.88 | 0.00; 21.00 | 7.00; 7.00 | 0.00; 9.00 | 0.00; 5.00 |
| Min; Max | 0.0; 21.0 | 0.0; 21.0 | 0.0; 21.0 | 0.0; 21.0 | 0.0; 21.0 | 7.0; 7.0 | 0.0; 18.0 | 0.0; 18.0 |
| Change from baseline | | | | | | | | |
| Number (observed/imputed) | 32 (31/1) | 30 (27/3) | 26 (24/2) | 28 (27/1) | 3 (2/1) | 1 (1/0) | 4 (3/1) | 5 (3/2) |
| Mean (SD) | −5.11 (6.70) | −5.69 (4.72) | −7.40 (6.12) | −10.27 (6.02) | −11.67 (10.69) | −14.00 (NC) | −13.50 (9.33) | −13.00 (9.27) |
| Median | −5.50 | −5.33 | −7.00 | −10.33 | −14.00 | −14.00 | −16.50 | −17.00 |
| Q1; Q3 | −9.00; −0.25 | −8.17; −1.40 | −11.00; −3.00 | −14.50; −4.63 | −21.00; 0.00 | −14.00; −14.00 | −19.50; −7.50 | −21.00; −3.00 |
| Min; Max | −17.5; 9.8 | −19.0; 1.0 | −20.0; 2.0 | −21.0; 0.0 | −21.0; 0.0 | −14.0; −14.0 | −21.0; 0.0 | −21.0; −3.0 |
| Percent change from | | | | | | | | |
| baseline | | | | | | | | |
| Number (observed/imputed) | 32 (31/1) | 30 (27/3) | 26 (24/2) | 28 (27/1) | 3 (2/1) | 1 (1/0) | 4 (3/1) | 5 (3/2) |
| Mean (SD) | −32.65 (46.69) | −33.81 (26.94) | −49.06 (38.85) | −62.66 (31.72) | −55.56 (50.92) | −66.67 (NC) | −75.00 (50.00) | −70.36 (41.41) |
| Median | −31.67 | −32.64 | −45.00 | −57.19 | −66.67 | −66.67 | −100.00 | −100.00 |
| Q1; Q3 | −66.23; −2.27 | −50.00; −8.33 | −91.67; −26.67 | −100.00; −34.52 | −100.00; 0.00 | −66.67; −66.67 | −100.00; −50.00 | −100.00; −37.50 |
| Min; Max | −100.0; 98.3 | −100.0; 5.0 | −100.0; 22.2 | −100.0; 0.0 | −100.0; 0.0 | −66.7; −66.7 | −100.0; 0.0 | −100.0; −14.3 |
| End of Study | | | | | | | | |
| Number (observed/imputed) | 5 (3/2) | 7 (3/4) | 5 (3/2) | 5 (3/2) | 0 (0/0) | 0 (0/0) | 0 (0/0) | 2 (0/2) |
| Mean (SD) | 12.17 (7.59) | 5.74 (8.03) | 7.80 (8.76) | 11.40 (6.11) | | | | 9.50 (6.36) |
| Median | 13.00 | 2.00 | 7.00 | 10.00 | | | | 9.50 |
| Q1; Q3 | 12.83; 14.00 | 0.00; 14.00 | 0.00; 11.00 | 7.00; 15.00 | | | | 5.00; 14.00 |
| Min; Max | 0.0; 21.0 | 0.0; 20.0 | 0.0; 21.0 | 5.0; 20.0 | | | | 5.0; 14.0 |
| Change from baseline | | | | | | | | |
| Number (observed/imputed) | 5 (3/2) | 7 (3/4) | 5 (3/2) | 5 (3/2) | 0 (0/0) | 0 (0/0) | 0 (0/0) | 2 (0/2) |
| Mean (SD) | −4.23 (5.39) | −11.40 (8.46) | −6.60 (7.33) | −6.20 (3.70) | | | | −1.50 (2.12) |
| Median | −7.00 | −14.00 | −8.00 | −7.00 | | | | −1.50 |
| Q1; Q3 | −7.00; 0.00 | −19.00; 0.00 | −13.00; 0.00 | −9.00; −6.00 | | | | −3.00; 0.00 |
| Min; Max | −10.0; 2.8 | −20.0; 0.0 | −14.0; 2.0 | 9.0; 0.0 | | | | −3.0; 0.0 |
| Percent change from | | | | | | | | |
| baseline | | | | | | | | |
| Number (observed/imputed) | 5 (3/2) | 7 (3/4) | 5 (3/2) | 5 (3/2) | 0 (0/0) | 0 (0/0) | 0 (0/0) | 2 (0/2) |
| Mean (SD) | −28.00 (48.01) | −66.19 (45.96) | −46.22 (56.25) | −38.05 (24.78) | | | | −18.75 (26.52) |
| Median | −33.33 | −83.33 | −53.33 | −47.37 | | | | −18.75 |
| Q1; Q3 | −35.00; 0.00 | −100.00; 0.00 | −100.00; 0.00 | −50.00; −28.57 | | | | −37.50; 0.00 |
| Min; Max | −100.0; 28.3 | −100.0; 0.0 | −100.0; 22.2 | −64.3; 0.0 | | | | −37.5; 0.0 |

WOCF: Worst observation carried forward
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF.

TABLE 29
Change and Percent Change from Baseline in ISS7 Over Time Up to Week 12 (ITT Population)

| | | | | Change | Change difference | | | % Change | |
|---|---|---|---|---|---|---|---|---|---|
| | | | | from | for rilzabrutinib | | % Change | difference for | |
| | | | Post- | baseline | vs. placebo | | from | rilzabrutinib vs. placebo | |
| | | Baseline | baseline | LS Mean | LS Mean | | baseline LS | LS Mean | |
| | n | Mean (SD) | Mean (SD) | (SE)a | (95% CI)^{a} | p-valueª | Mean (SE)ª | (95% CI)^{a} | p-valueª |
| Week 1 | | | | | | | | | |
| Rilzabrutinib 400 | 41 | 15.88 (4.37) | 9.20 (5.60) | −6.65 (0.76) | −4.65 | <.0001 | −40.41 (5.24) | −29.95 | <.0001 |
| mg TID (N = 41) | | | | | (−7.14, −2.15) | | | (−44.08, −15.81) | |
| Rilzabrutinib 400 | 41 | 15.80 (3.68) | 10.65 (5.86) | −5.23 (0.78) | −3.23 | 0.0024 | −34.02 (5.28) | −23.55 | 0.0009 |
| mg BID (N = 41) | | | | | (−5.72, −0.74) | | | (−37.52, −9.59) | |
| Rilzabrutinib 400 | 38 | 16.50 (3.53) | 13.08 (4.46) | −3.47 (0.79) | −1.47 | 0.1716 | −19.33 (5.35) | −8.87 | 0.2225 |
| mg QPM (N = 38) | | | | | (−4.00, 1.07) | | | (−23.12, 5.38) | |
| Placebo (N = 40) | 40 | 15.60 (4.24) | 13.91 (5.26) | −2.00 (0.78) | | | −10.46 (5.30) | | |
| Week 2 | | | | | | | | | |
| Rilzabrutinib 400 | 41 | 15.88 (4.37) | 7.97 (5.99) | −7.92 (0.79) | −4.51 | <.0001 | −49.29 (5.21) | −29.81 | <.0001 |
| mg TID (N = 41) | | | | | (−6.65, −2.38) | | | (−43.92, −15.69) | |
| Rilzabrutinib 400 | 40 | 15.80 (3.68) | 10.40 (5.75) | −5.68 (0.80) | −2.27 | 0.0363 | −36.11 (5.26) | −16.63 | 0.0192 |
| mg BID (N = 41) | | | | | (−4.39, −0.14) | | | (−30.54, −2.71) | |
| Rilzabrutinib 400 | 38 | 16.50 (3.53) | 12.44 (4.70) | −4.27 (0.81) | −0.86 | 0.4345 | −25.13 (5.32) | −5.64 | 0.4360 |
| mg QPM (N = 38) | | | | | (−3.03, 1.30) | | | (−19.84, 8.56) | |
| Placebo (N = 40) | 39 | 15.60 (4.24) | 12.61 (5.31) | −3.41 (0.81) | | | −19.49 (5.28) | | |
| Week 3 | | | | | | | | | |
| Rilzabrutinib 400 | 41 | 15.88 (4.37) | 7.93 (5.46) | −7.84 (0.83) | −3.76 | 0.0010 | −50.07 (5.21) | −27.63 | 0.0001 |
| mg TID (N = 41) | | | | | (−6.00, −1.51) | | | (−41.72, −13.53) | |
| Rilzabrutinib 400 | 41 | 15.80 (3.68) | 9.21 (6.17) | −6.68 (0.84) | −2.60 | 0.0225 | −40.88 (5.26) | −18.44 | 0.0095 |
| mg BID (N = 41) | | | | | (−4.83, −0.37) | | | (−32.38, −4.50) | |
| Rilzabrutinib 400 | 38 | 16.50 (3.53) | 11.54 (4.63) | −5.06 (0.85) | −0.97 | 0.4036 | −29.78 (5.33) | −7.34 | 0.3125 |
| mg QPM (N = 38) | | | | | (−3.25, 1.31) | | | (−21.57, 6.90) | |
| Placebo (N = 40) | 39 | 15.60 (4.24) | 11.94 (5.42) | −4.08 (0.85) | | | −22.44 (5.30) | | |
| Week 4 | | | | | | | | | |
| Rilzabrutinib 400 | 38 | 15.88 (4.37) | 7.54 (5.21) | −7.75 (0.88) | −3.13 | 0.0096 | −48.97 (5.57) | −23.20 | 0.0027 |
| mg TID (N = 41) | | | | | (−5.50, −0.76) | | | (−38.37, −8.04) | |
| Rilzabrutinib 400 | 38 | 15.80 (3.68) | 8.12 (6.28) | −7.24 (0.89) | −2.62 | 0.0291 | −44.30 (5.62) | −18.54 | 0.0147 |
| mg BID (N = 41) | | | | | (−4.98, −0.27) | | | (−33.43, −3.64) | |
| Rilzabrutinib 400 | 38 | 16.50 (3.53) | 11.30 (4.76) | −5.29 (0.90) | −0.67 | 0.5838 | −30.86 (5.64) | −5.09 | 0.5109 |
| mg QPM (N = 38) | | | | | (−3.07, 1.73) | | | (−20.26, 10.08) | |
| Placebo (N = 40) | 38 | 15.60 (4.24) | 11.34 (6.01) | −4.62 (0.90) | | | −25.77 (5.66) | | |
| Week 5 | | | | | | | | | |
| Rilzabrutinib 400 | 39 | 15.88 (4.37) | 6.70 (5.91) | −8.93 (0.86) | −3.36 | 0.0046 | −54.96 (5.32) | −21.69 | 0.0033 |
| mg TID (N = 41) | | | | | (−5.69, −1.04) | | | (−36.14, −7.23) | |
| Rilzabrutinib 400 | 38 | 15.80 (3.68) | 8.53 (6.95) | −6.89 (0.88) | −1.32 | 0.2653 | 43.12 (5.39) | −9.84 | 0.1769 |
| mg BID (N = 41) | | | | | (−3.65, 1.00) | | | (−24.14, 4.45) | |
| Rilzabrutinib 400 | 37 | 16.50 (3.53) | 11.00 (4.61) | −5.48 (0.88) | 0.08 | 0.9441 | −32.42 (5.42) | 0.86 | 0.9079 |
| mg QPM (N = 38) | | | | | (−2.27, 2.44) | | | (−13.68, 15.40) | |
| Placebo (N = 40) | 37 | 15.60 (4.24) | 10.14 (4.82) | −5.57 (0.88) | | | −33.27 (5.42) | | |
| Week 6 | | | | | | | | | |
| Rilzabrutinib 400 | 37 | 15.88 (4.37) | 6.81 (6.20) | −8.97 (0.93) | −4.09 | 0.0014 | −55.31 (5.83) | −26.88 | 0.0009 |
| mg TID (N = 41) | | | | | (−6.60, −1.57) | | | (−42.81, −10.95) | |
| Rilzabrutinib 400 | 39 | 15.80 (3.68) | 8.62 (6.58) | −6.87 (0.93) | −1.99 | 0.1157 | −42.18 (5.84) | −13.75 | 0.0841 |
| mg BID (N = 41) | | | | | (−4.47, 0.49) | | | (−29.35, 1.85) | |
| Rilzabrutinib 400 | 35 | 16.50 (3.53) | 10.23 (5.19) | −6.28 (0.95) | −1.39 | 0.2855 | −37.07 (5.96) | −8.64 | 0.2892 |
| mg QPM (N = 38) | | | | | (−3.95, 1.16) | | | (−24.62, 7.34) | |
| Placebo (N = 40) | 36 | 15.60 (4.24) | 10.87 (5.82) | −4.89 (0.95) | | | −28.43 (5.99) | | |
| Week 7 | | | | | | | | | |
| Rilzabrutinib 400 | 36 | 15.88 (4.37) | 7.13 (5.42) | −8.71 (0.91) | −3.71 | 0.0028 | −53.83 (5.69) | −24.96 | 0.0014 |
| mg TID (N = 41) | | | | | (−6.15, −1.27) | | | (−40.31, −9.62) | |
| Rilzabrutinib 400 | 39 | 15.80 (3.68) | 8.03 (6.39) | −7.77 (0.91) | −2.77 | 0.0243 | −47.79 (5.68) | −18.93 | 0.0138 |
| mg BID (N = 41) | | | | | (−5.18, −0.36) | | | (−34.00, −3.86) | |
| Rilzabrutinib 400 | 34 | 16.50 (3.53) | 10.60 (5.27) | −6.37 (0.93) | −1.37 | 0.2799 | −38.52 (5.84) | −9.66 | 0.2269 |
| mg QPM (N = 38) | | | | | (−3.86, 1.12) | | | (−25.33, 6.01) | |
| Placebo (N = 40) | 37 | 15.60 (4.24) | 10.98 (5.74) | −5.00 (0.92) | | | −28.86 (5.76) | | |
| Week 8 | | | | | | | | | |
| Rilzabrutinib 400 | 34 | 15.88 (4.37) | 6.72 (5.76) | −8.66 (0.95) | −3.05 | 0.0198 | −54.76 (5.95) | −21.97 | 0.0073 |
| mg TID (N = 41) | | | | | (−5.61, −0.48) | | | (−38.02, −5.91) | |
| Rilzabrutinib 400 | 38 | 15.80 (3.68) | 7.18 (6.69) | −8.47 (0.93) | −2.86 | 0.0243 | −52.80 (5.92) | −20.00 | 0.0127 |
| mg BID (N = 41) | | | | | (−5.34, −0.37) | | | (−35.73, −4.26) | |
| Rilzabrutinib 400 | 34 | 16.50 (3.53) | 10.61 (5.22) | −6.22 (0.96) | −0.61 | 0.6407 | −38.39 (6.03) | −5.60 | 0.4963 |
| mg QPM (N = 38) | | | | | (−3.18, 1.96) | | | (−21.72, 10.53) | |
| Placebo (N = 40) | 37 | 15.60 (4.24) | 10.34 (6.19) | −5.61 (0.94) | | | −32.80 (5.95) | | |
| Week 9 | | | | | | | | | |
| Rilzabrutinib 400 | 35 | 15.88 (4.37) | 6.33 (5.52) | −8.85 (0.95) | −3.35 | 0.0107 | −55.43 (6.01) | −24.57 | 0.0030 |
| mg TID (N = 41) | | | | | (−5.93, −0.78) | | | (−40.80, −8.33) | |
| Rilzabrutinib 400 | 34 | 15.80 (3.68) | 7.44 (7.20) | −8.27 (0.96) | −2.77 | 0.0337 | −51.80 (5.94) | −20.94 | 0.0091 |
| mg BID (N = 41) | | | | | (−5.33, −0.21) | | | (−36.69, −5.20) | |
| Rilzabrutinib 400 | 35 | 16.50 (3.53) | 10.45 (5.08) | −6.24 (0.96) | −0.74 | 0.5717 | −37.93 | −7.07 | 0.3920 |
| mg QPM (N = 38) | | | | | (−3.32, 1.83) | | (6.02) | (−23.26, 9.12) | |
| Placebo (N = 40) | 36 | 15.60 (4.24) | 10.54 (5.97) | −5.50 (0.96) | | | −30.86 (5.97) | | |
| Week 10 | | | | | | | | | |
| Rilzabrutinib 400 | 36 | 15.88 (4.37) | 6.43 (5.48) | −8.98 (0.99) | −2.68 | 0.0496 | −56.23 (6.14) | −18.42 | 0.0293 |
| mg TID (N = 41) | | | | | (−5.36, −0.00) | | | (−34.98, −1.86) | |
| Rilzabrutinib 400 | 33 | 15.80 (3.68) | 8.06 (7.29) | −8.01 (1.01) | −1.72 | 0.2088 | −48.75 (6.31) | −10.95 | 0.1939 |
| mg BID (N = 41) | | | | | (−4.39, 0.96) | | | (−27.46, 5.57) | |
| Rilzabrutinib 400 | 35 | 16.50 (3.53) | 10.36 (5.24) | −6.47 (1.02) | −0.17 | 0.9030 | −39.07 (6.29) | −1.26 | 0.8835 |
| mg QPM (N = 38) | | | | | (−2.89, 2.55) | | | (−18.15, 15.62) | |
| Placebo (N = 40) | 37 | 15.60 (4.24) | 9.82 (6.66) | −6.30 (1.01) | | | −37.81 (6.23) | | |
| Week 11 | | | | | | | | | |
| Rilzabrutinib 400 | 37 | 15.88 (4.37) | 6.57 (5.76) | −9.02 (0.96) | −3.15 | 0.0173 | −55.75 (6.15) | −22.13 | 0.0094 |
| mg TID (N = 41) | | | | | (−5.75, −0.56) | | | (−38.82, −5.44) | |
| Rilzabrutinib 400 | 34 | 15.80 (3.68) | 7.76 (7.01) | −7.93 (0.99) | −2.07 | 0.1169 | −49.45 (6.23) | −15.82 | 0.0601 |
| mg BID (N = 41) | | | | | (−4.65, 0.52) | | | (−32.32, 0.67) | |
| Rilzabrutinib 400 | 34 | 16.50 (3.53) | 10.97 (5.48) | −5.90 (0.99) | −0.04 | 0.9783 | −36.92 (6.23) | −3.30 | 0.6977 |
| mg QPM (N = 38) | | | | | (−2.69, 2.62) | | | (−19.96, 13.36) | |
| Placebo (N = 40) | 37 | 15.60 (4.24) | 10.09 (6.46) | −5.87 (0.98) | | | −33.62 (6.18) | | |
| Week 12 | | | | | | | | | |
| Rilzabrutinib 400 | 33 | 15.88 (4.37) | 5.59 (5.58) | −9.58 (1.01) | −3.26 | 0.0181 | −59.81 (6.28) | −23.23 | 0.0070 |
| mg TID (N = 41) | | | | | (−5.97, −0.56) | | | (−40.11, −6.35) | |
| Rilzabrutinib 400 | 30 | 15.80 (3.68) | 7.61 (7.18) | −8.06 (1.02) | −1.75 | 0.2041 | −50.27 (6.54) | −13.69 | 0.1176 |
| mg BID (N = 41) | | | | | (−4.46, 0.95) | | | (−30.83, 3.46) | |
| Rilzabrutinib 400 | 31 | 16.50 (3.53) | 11.37 (5.51) | −5.46 (1.03) | 0.85 | 0.5439 | −35.23 (6.55) | 1.35 | 0.8790 |
| mg QPM (N = 38) | | | | | (−1.90, 3.60) | | | (−16.05, 18.76) | |
| Placebo (N = 40) | 35 | 15.60 (4.24) | 9.96 (7.02) | −6.31 (1.02) | | | −36.58 (6.30) | | |

^{a}Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF; other missing data were imputed by MI. Descriptive statistics at each week include participants with WOCF imputation at the week, and participants whose values were imputed by MI at the week were excluded from the descriptive analysis.

TABLE 30
Change and Percent Change from Baseline in ISS7 over Time up to Week 12 (Omalizumab-Naïve Population)

| | | | | | Change difference | | | | |
|---|---|---|---|---|---|---|---|---|---|
| | | | | | for rilzabrutinib | | % Change | % Change difference for | |
| | | | Post- | Change from | vs. placebo | | from baseline | rilzabrutinib vs. placebo | |
| | | Baseline | baseline | baseline LS | LS Mean | | LS Mean | LS Mean | |
| | n | Mean (SD) | Mean (SD) | Mean (SE)^{a} | (95% CI)ª | p-valueª | (SE)^{a} | (95% CI)^{a} | p-valueª |
| Week 1 | | | | | | | | | |
| Rilzabrutinib 400 | 35 | 15.69 (4.26) | 9.51 (5.64) | −6.13 (0.82) | −4.21 | 0.0003 | −37.45 (5.78) | −27.33 (−42.78, −11.87) | 0.0005 |
| mg TID (N = 35) | | | | | (−6.89, −1.52) | | | | |
| Rilzabrutinib 400 | 35 | 15.45 (3.72) | 10.51 (5.88) | −5.06 (0.83) | −3.14 | 0.0060 | −33.92 (5.73) | −23.80 (−38.99, −8.60) | 0.0021 |
| mg BID (N = 35) | | | | | (−5.80, −0.47) | | | | |
| Rilzabrutinib 400 | 37 | 16.38 (3.50) | 13.08 (4.52) | −3.25 (0.80) | −1.33 | 0.2347 | −18.38 (5.55) | −8.25 (−23.30, 6.80) | 0.2827 |
| mg QPM (N = 37) | | | | | (−3.97, 1.31) | | | | |
| Placebo (N = 36) | 36 | 15.14 (4.16) | 13.57 (5.31) | −1.93 (0.82) | | | −10.12 (5.66) | | |
| Week 2 | | | | | | | | | |
| Rilzabrutinib 400 | 35 | 15.69 (4.26) | 8.40 (5.82) | −7.37 (0.82) | −4.14 | 0.0002 | −46.27 (5.61) | −27.63 (−42.70, −12.56) | 0.0003 |
| mg TID (N = 35) | | | | | (−6.35, −1.94) | | | | |
| Rilzabrutinib 400 | 34 | 15.45 (3.72) | 10.32 (5.64) | −5.49 (0.83) | −2.26 | 0.0431 | −35.92 (5.58) | −17.28 (−32.06, −2.50) | 0.0219 |
| mg BID (N = 35) | | | | | (−4.46, −0.07) | | | | |
| Rilzabrutinib 400 | 37 | 16.38 (3.50) | 12.53 (4.73) | −3.99 (0.80) | −0.76 | 0.4948 | −23.96 (5.37) | −5.32 (−19.96, 9.32) | 0.4765 |
| mg QPM (N = 37) | | | | | (−2.92, 1.41) | | | | |
| Placebo (N = 36) | 35 | 15.14 (4.16) | 12.38 (5.17) | −3.23 (0.82) | | | −18.64 (5.51) | | |
| Week 3 | | | | | | | | | |
| Rilzabrutinib 400 | 35 | 15.69 (4.26) | 8.34 (5.42) | −7.29 (0.87) | −3.58 | 0.0027 | −47.81 (5.64) | −27.20 (−42.33, −12.08) | 0.0004 |
| mg TID (N = 35) | | | | | (−5.93, −1.24) | | | | |
| Rilzabrutinib 400 | 35 | 15.45 (3.72) | 9.16 (6.09) | −6.52 (0.87) | −2.82 | 0.0175 | −40.91 (5.60) | −20.30 (−35.19, −5.41) | 0.0076 |
| mg BID (N = 35) | | | | | (−5.15, −0.49) | | | | |
| Rilzabrutinib 400 | 37 | 16.38 (3.50) | 11.60 (4.68) | −4.82 (0.85) | −1.12 | 0.3399 | −28.99 (5.42) | −8.38 (−23.14, 6.38) | 0.2659 |
| mg QPM (N = 37) | | | | | (−3.43, 1.18) | | | | |
| Placebo (N = 36) | 35 | 15.14 (4.16) | 11.98 (5.40) | −3.70 (0.87) | | | −20.61 (5.56) | | |
| Week 4 | | | | | | | | | |
| Rilzabrutinib 400 | 32 | 15.69 (4.26) | 7.79 (5.03) | −7.33 (0.92) | −3.23 | 0.0106 | −47.50 (6.02) | −24.55 (−40.74, −8.36) | 0.0030 |
| mg TID (N = 35) | | | | | (−5.71, −0.75) | | | | |
| Rilzabrutinib 400 | 33 | 15.45 (3.72) | 8.32 (6.02) | −6.96 (0.93) | −2.86 | 0.0227 | −43.47 (5.96) | −20.53 (−36.37, −4.69) | 0.0111 |
| mg BID (N = 35) | | | | | (−5.32, −0.40) | | | | |
| Rilzabrutinib 400 | 37 | 16.38 (3.50) | 11.39 (4.79) | −5.03 (0.89) | −0.93 | 0.4535 | −29.97 (5.75) | −7.02 (−22.76, 8.71) | 0.3817 |
| mg QPM (N = 37) | | | | | (−3.36, 1.50) | | | | |
| Placebo (N = 36) | 34 | 15.14 (4.16) | 11.51 (6.14) | −4.10 (0.92) | | | −22.94 (5.94) | | |
| Week 5 | | | | | | | | | |
| Rilzabrutinib 400 | 33 | 15.69 (4.26) | 6.92 (5.87) | −8.51 (0.91) | −2.88 | 0.0216 | −53.64 (5.75) | −19.52 (−35.10, −3.93) | 0.0141 |
| mg TID (N = 35) | | | | | (−5.34, −0.42) | | | | |
| Rilzabrutinib 400 | 34 | 15.45 (3.72) | 8.88 (6.81) | −6.55 (0.91) | −0.92 | 0.4561 | −42.17 (5.73) | −8.04 (−23.43, 7.34) | 0.3055 |
| mg BID (N = 35) | | | | | (−3.33, 1.50) | | | | |
| Rilzabrutinib 400 | 36 | 16.38 (3.50) | 11.00 (4.68) | −5.30 (0.88) | 0.33 | 0.7894 | −31.89 (5.56) | 2.23 (−13.08, 17.55) | 0.7751 |
| mg QPM (N = 37) | | | | | (−2.07, 2.73) | | | | |
| Placebo (N = 36) | 33 | 15.14 (4.16) | 9.75 (4.75) | −5.63 (0.91) | | | −34.12 (5.78) | | |
| Week 6 | | | | | | | | | |
| Rilzabrutinib 400 | 31 | 15.69 (4.26) | .32 (6.05) | −8.27 (0.99) | −3.47 | 0.0103 | −52.36 (6.28) | −24.70 (−41.62, −7.79) | 0.0042 |
| mg TID (N = 35) | | | | | (−6.12, −0.82) | | | | |
| Rilzabrutinib 400 | 34 | 15.45 (3.72) | 8.85 (6.40) | −6.57 (0.97) | −1.76 | 0.1834 | −41.28 (6.23) | −13.63 (−30.25, 3.00) | 0.1082 |
| mg BID (N = 35) | | | | | (−4.36, 0.83) | | | | |
| Rilzabrutinib 400 | 34 | 16.38 (3.50) | 10.27 (5.26) | −6.02 (0.95) | −1.21 | 0.3604 | −36.29 (6.07) | −8.63 (−25.16, 7.90) | 0.3060 |
| mg QPM (N = 37) | | | | | (−3.81, 1.39) | | | | |
| Placebo (N = 36) | 32 | 15.14 (4.16) | 10.58 (5.85) | −4.80 (0.98) | | | −27.66 (6.23) | | |
| Week 7 | | | | | | | | | |
| Rilzabrutinib 400 | 30 | 15.69 (4.26) | 7.14 (5.35) | −8.54 (0.97) | −3.43 | 0.0091 | −54.42 (6.29) | −24.69 (−41.39, −7.99) | 0.0038 |
| mg TID (N = 35) | | | | | (−6.01, −0.85) | | | | |
| Rilzabrutinib 400 | 34 | 15.45 (3.72) | 8.21 (6.16) | −7.47 (0.94) | −2.36 | 0.0664 | −46.77 (6.08) | −17.04 (−33.33, −0.75) | 0.0404 |
| mg BID (N = 35) | | | | | (−4.88, 0.16) | | | | |
| Rilzabrutinib 400 | 33 | 16.38 (3.50) | 10.68 (5.34) | −6.16 (0.93) | −1.06 | 0.4144 | −38.23 (5.98) | −8.51 (−24.78, 7.77) | 0.3057 |
| mg QPM (N = 37) | | | | | (−3.59, 1.48) | | | | |
| Placebo (N = 36) | 33 | 15.14 (4.16) | 10.51 (5.70) | −5.11 (0.95) | | | −29.73 (6.10) | | |
| Week 8 | | | | | | | | | |
| Rilzabrutinib 400 | 28 | 15.69 (4.26) | 7.24 (5.51) | −7.93 (1.03) | −2.52 | 0.0697 | −51.09 (6.57) | −19.18 (−36.57, −1.80) | 0.0306 |
| mg TID (N = 35) | | | | | (−5.25, 0.20) | | | | |
| Rilzabrutinib 400 | 33 | 15.45 (3.72) | 7.33 (6.55) | −8.20 (0.98) | −2.79 | 0.0363 | −52.58 (6.34) | −20.68 (−37.61, −3.74) | 0.0167 |
| mg BID (N = 35) | | | | | (−5.41, −0.18) | | | | |
| Rilzabrutinib 400 | 33 | 16.38 (3.50) | 10.72 (5.26) | −5.96 (0.96) | −0.55 | 0.6792 | −37.54 (6.29) | −5.64 (−22.51, 11.23) | 0.5122 |
| mg QPM (N = 37) | | | | | (−3.17, 2.06) | | | | |
| Placebo (N = 36) | 33 | 15.14 (4.16) | 10.15 (6.23) | −5.41 (0.98) | | | −31.90 (6.28) | | |
| Week 9 | | | | | | | | | |
| Rilzabrutinib 400 | 29 | 15.69 (4.26) | 6.68 (5.31) | −8.45 (1.00) | −3.43 | 0.0114 | −53.94 (6.32) | −24.44 (−41.44, −7.44) | 0.0048 |
| mg TID (N = 35) | | | | | (−6.09, −0.77) | | | | |
| Rilzabrutinib 400 | 29 | 15.45 (3.72) | 7.63 (7.11) | −7.96 (0.99) | −2.94 | 0.0282 | −51.70 (6.32) | −22.21 (−38.97, −5.44) | 0.0094 |
| mg BID (N = 35) | | | | | (−5.57, −0.32) | | | | |
| Rilzabrutinib 400 | 34 | 16.38 (3.50) | 10.46 (5.15) | −6.10 (0.96) | −1.08 | 0.4202 | −37.27 (6.13) | −7.78 (−24.44, 8.89) | 0.3604 |
| mg QPM (N = 37) | | | | | (−3.70, 1.54) | | | | |
| Placebo (N = 36) | 32 | 15.14 (4.16) | 10.55 (5.94) | −5.02 (0.98) | | | −29.49 (6.26) | | |
| Week 10 | | | | | | | | | |
| Rilzabrutinib 400 | 30 | 15.69 (4.26) | 6.75 (5.25) | −8.59 (1.03) | −2.91 | 0.0400 | −54.88 (6.72) | −20.32 (−38.47, −2.16) | 0.0283 |
| mg TID (N = 35) | | | | | (−5.68, −0.13) | | | | |
| Rilzabrutinib 400 | 28 | 15.45 (3.72) | 8.17 (7.29) | −7.77 (1.05) | −2.09 | 0.1405 | −48.38 (6.69) | −13.81 (−31.52, 3.91) | 0.1267 |
| mg BID (N = 35) | | | | | (−4.86, 0.69) | | | | |
| Rilzabrutinib 400 | 34 | 16.38 (3.50) | 10.43 (5.30) | −6.26 (1.01) | −0.58 | 0.6786 | −38.74 (6.43) | −4.17 (−21.94, 13.59) | 0.6454 |
| mg QPM (N = 37) | | | | | (−3.31, 2.15) | | | | |
| Placebo (N = 36) | 33 | 15.14 (4.16) | 10.00 (6.49) | −5.68 (1.02) | | | −34.57 (6.68) | | |
| Week 11 | | | | | | | | | |
| Rilzabrutinib 400 | 31 | 15.69 (4.26) | 6.99 (5.53) | −8.50 (1.02) | −3.19 | 0.0224 | −53.92 (6.66) | −22.62 (−40.61, −4.64) | 0.0137 |
| mg TID (N = 35) | | | | | (−5.92, −0.45) | | | | |
| Rilzabrutinib 400 | 29 | 15.45 (3.72) | 7.93 (6.94) | −7.75 (1.02) | −2.43 | 0.0790 | −49.16 (6.63) | −17.86 (−35.62, −0.09) | 0.0488 |
| mg BID (N = 35) | | | | | (−5.15, 0.28) | | | | |
| Rilzabrutinib 400 | 33 | 16.38 (3.50) | 11.03 (5.55) | −5.73 (1.00) | −0.42 | 0.7628 | −36.17 (6.53) | −4.87 (−22.51, 12.77) | 0.5885 |
| mg QPM (N = 37) | | | | | (−3.12, 2.28) | | | | |
| Placebo (N = 36) | 33 | 15.14 (4.16) | 10.24 (6.31) | −5.32 (1.01) | | | −31.30 (6.59) | | |
| Week 12 | | | | | | | | | |
| Rilzabrutinib 400 | 28 | 15.69 (4.26) | 5.77 (5.26) | −9.21 (1.05) | −3.44 | 0.0168 | −58.05 (6.73) | −23.84 (−41.97, −5.71) | 0.0100 |
| mg TID (N = 35) | | | | | (−6.25, −0.62) | | | | |
| Rilzabrutinib 400 | 26 | 15.45 (3.72) | 8.09 (6.95) | −7.73 (1.06) | −1.96 | 0.1728 | −48.31 (6.75) | −14.10 (−32.26, 4.06) | 0.1281 |
| mg BID (N = 35) | | | | | (−4.77, 0.86) | | | | |
| Rilzabrutinib 400 | 30 | 16.38 (3.50) | 11.51 (5.54) | −5.19 (1.02) | 0.58 | 0.6869 | −34.75 (6.64) | −0.54 (−18.55, 17.47) | 0.9529 |
| mg QPM (N = 37) | | | | | (−2.24, 3.40) | | | | |
| Placebo (N = 36) | 32 | 15.14 (4.16) | 10.02 (6.82) | −5.77 (1.05) | | | −34.21 (6.72) | | |

^{a}Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF; other missing data were imputed by MI. Descriptive statistics at each week include participants with WOCF imputation at the week, and participants whose values were imputed by MI at the week were excluded from the descriptive analysis.

TABLE 31
Change and Percent Change from Baseline in ISS7 Over Time Up to Week 12 (Omalizumab-Naïve Population with
Outlier Removed from Analysis)

| | | | | | Change difference | | | % Change difference | |
|---|---|---|---|---|---|---|---|---|---|
| | | | | | for rilzabrutinib | | % Change | for rilzabrutinib | |
| | | | Post- | Change from | vs. placebo | | from | vs. placebo | |
| | | Baseline | baseline | baseline LS | LS Mean | p- | baseline LS | LS Mean | p- |
| | n | Mean (SD) | Mean (SD) | Mean (SE)^{a} | (95% CI)^{a} | valueª | Mean (SE)ª | (95% CI)ª | valueª |
| Week 1 | | | | | | | | | |
| Rilzabrutinib 400 | 34 | 16.15 (3.32) | 9.79 (5.47) | −6.21 (0.84) | −4.25 (−6.97, −1.53) | 0.0003 | −37.73 (5.76) | −27.54 (−46.26, −8.82) | 0.0006 |
| mg TID (N = 34) | | | | | | | | | |
| Rilzabrutinib 400 | 35 | 15.45 (3.72) | 10.51 (5.88) | −5.10 (0.83) | −3.14 (−5.82, −0.46) | 0.0062 | −34.00 (5.73) | −23.81 (−42.23, −5.39) | 0.0026 |
| mg BID (N = 35) | | | | | | | | | |
| Rilzabrutinib 400 | 37 | 16.38 (3.50) | 13.08 (4.52) | −3.30 (0.81) | −1.34 (−3.99, 1.31) | 0.2324 | −18.50 (5.54) | −8.32 (−26.56, 9.93) | 0.2807 |
| mg QPM (N = 37) | | | | | | | | | |
| Placebo (N = 36) | 36 | 15.14 (4.16) | 13.57 (5.31) | −1.96 (0.82) | | | −10.19 (5.67) | | |
| Week 2 | | | | | | | | | |
| Rilzabrutinib 400 | 34 | 16.15 (3.32) | 8.59 (5.80) | −7.53 (0.83) | −4.29 (−6.52, −2.06) | 0.0002 | −46.27 (5.57) | −27.63 (−42.59, −12.68) | 0.0003 |
| mg TID (N = 34) | | | | | | | | | |
| Rilzabrutinib 400 | 34 | 15.45 (3.72) | 10.32 (5.64) | −5.52 (0.83) | −2.27 (−4.47, −0.08) | 0.0425 | −36.28 (5.56) | −17.64 (−32.37, −2.92) | 0.0189 |
| mg BID (N = 35) | | | | | | | | | |
| Rilzabrutinib 400 | 37 | 16.38 (3.50) | 12.53 (4.73) | −4.05 (0.80) | −0.80 (−2.97, 1.37) | 0.4691 | −24.02 (5.36) | −5.38 (−19.96, 9.19) | 0.4692 |
| mg QPM (N = 37) | | | | | | | | | |
| Placebo (N = 36) | 35 | 15.14 (4.16) | 12.38 (5.17) | −3.24 (0.82) | | | −18.64 (5.49) | | |
| Week 3 | | | | | | | | | |
| Rilzabrutinib 400 | 34 | 16.15 (3.32) | 8.18 (5.41) | −7.77 (0.85) | −4.12 (−6.42, −1.83) | 0.0004 | −47.94 (5.64) | −27.10 (−42.26, −11.94) | 0.0005 |
| mg TID (N = 34) | | | | | | | | | |
| Rilzabrutinib 400 | 35 | 15.45 (3.72) | 9.16 (6.09) | −6.51 (0.85) | −2.87 (−5.12, −0.61) | 0.0127 | −41.02 (5.61) | −20.18 (−35.09, −5.26) | 0.0080 |
| mg BID (N = 35) | | | | | | | | | |
| Rilzabrutinib 400 | 37 | 16.38 (3.50) | 11.60 (4.68) | −4.94 (0.82) | −1.30 (−3.54, 0.94) | 0.2544 | −29.09 (5.42) | −8.25 (−23.04, 6.55) | 0.2746 |
| mg QPM (N = 37) | | | | | | | | | |
| Placebo (N = 36) | 35 | 15.14 (4.16) | 11.98 (5.40) | −3.64 (0.84) | | | −20.84 (5.58) | | |
| Week 4 | | | | | | | | | |
| Rilzabrutinib 400 | 32 | 16.15 (3.32) | 7.79 (5.03) | −7.78 (0.91) | −3.69 (−6.13, −1.25) | 0.0031 | −47.66 (6.02) | −24.30 (−40.44, −8.16) | 0.0032 |
| mg TID (N = 34) | | | | | | | | | |
| Rilzabrutinib 400 | 33 | 15.45 (3.72) | 8.32 (6.02) | −6.96 (0.91) | −2.86 (−5.26, −0.46) | 0.0193 | −43.56 (5.98) | −20.20 (−36.06, −4.35) | 0.0125 |
| mg BID (N = 35) | | | | | | | | | |
| Rilzabrutinib 400 | 37 | 16.38 (3.50) | 11.39 (4.79) | −5.14 (0.87) | −1.05 (−3.42, 1.32) | 0.3841 | −30.05 (5.74) | −6.69 (−22.35, 8.98) | 0.4028 |
| mg QPM (N = 37) | | | | | | | | | |
| Placebo (N = 36) | 34 | 15.14 (4.16) | 11.51 (6.14) | −4.09 (0.89) | | | −23.36 (5.91) | | |
| Week 5 | | | | | | | | | |
| Rilzabrutinib 400 | 33 | 16.15 (3.32) | 6.92 (5.87) | −8.85 (0.91) | −3.21 (−5.65, −0.77) | 0.0100 | −53.59 (5.78) | −19.54 (−35.11, −3.96) | 0.0140 |
| mg TID (N = 34) | | | | | | | | | |
| Rilzabrutinib 400 | 34 | 15.45 (3.72) | 8.88 (6.81) | −6.55 (0.90) | −0.91 (−3.31, 1.48) | 0.4553 | −42.26 (5.73) | −8.21 (−23.48, 7.06) | 0.2920 |
| mg BID (N = 35) | | | | | | | | | |
| Rilzabrutinib 400 | 36 | 16.38 (3.50) | 11.00 (4.68) | −5.44 (0.87) | 0.20 (−2.17, 2.58) | 0.8663 | −32.18 (5.54) | 1.87 (−13.30, 17.05) | 0.8087 |
| mg QPM (N = 37) | | | | | | | | | |
| Placebo (N = 36) | 33 | 15.14 (4.16) | 9.75 (4.75) | −5.64 (0.90) | | | −34.06 (5.74) | | |
| Week 6 | | | | | | | | | |
| Rilzabrutinib 400 | 31 | 16.15 (3.32) | 7.32 (6.05) | −8.61 (0.98) | −3.77 (−6.39, −1.15) | 0.0047 | −52.19 (6.28) | −23.97 (−40.79, −7.15) | 0.0052 |
| mg TID (N = 34) | | | | | | | | | |
| Rilzabrutinib 400 | 34 | 15.45 (3.72) | 8.85 (6.40) | −6.56 (0.97) | −1.72 (−4.30, 0.87) | 0.1928 | −41.38 (6.22) | −13.16 (−29.74, 3.43) | 0.1201 |
| mg BID (N = 35) | | | | | | | | | |
| Rilzabrutinib 400 | 34 | 16.38 (3.50) | 10.27 (5.26) | −6.13 (0.94) | −1.29 (−3.88, 1.30) | 0.3299 | −36.45 (6.05) | −8.22 (−24.84, 8.40) | 0.3323 |
| mg QPM (N = 37) | | | | | | | | | |
| Placebo (N = 36) | 32 | 15.14 (4.16) | 10.58 (5.85) | −4.84 (0.98) | | | −28.23 (6.27) | | |
| Week 7 | | | | | | | | | |
| Rilzabrutinib 400 | 30 | 16.15 (3.32) | 7.14 (5.35) | −8.83 (0.95) | −3.69 (−6.24, −1.13) | 0.0046 | −54.22 (6.16) | −24.40 (−40.95, −7.85) | 0.0039 |
| mg TID (N = 34) | | | | | | | | | |
| Rilzabrutinib 400 | 34 | 15.45 (3.72) | 8.21 (6.16) | −7.50 (0.93) | −2.36 (−4.85, 0.14) | 0.0640 | −47.03 (6.06) | −17.21 (−33.40, −1.02) | 0.0372 |
| mg BID (N = 35) | | | | | | | | | |
| Rilzabrutinib 400 | 33 | 16.38 (3.50) | 10.68 (5.34) | −6.23 (0.91) | −1.09 (−3.58, 1.41) | 0.3929 | −38.17 (5.94) | −8.35 (−24.56, 7.86) | 0.3129 |
| mg QPM (N = 37) | | | | | | | | | |
| Placebo (N = 36) | 33 | 15.14 (4.16) | 10.51 (5.70) | −5.14 (0.94) | | | −29.82 (6.07) | | |
| Week 8 | | | | | | | | | |
| Rilzabrutinib 400 | 28 | 16.15 (3.32) | 7.24 (5.51) | −8.34 (0.99) | −2.89 (−5.57, −0.21) | 0.0343 | −51.64 (6.49) | −19.50 (−37.05, −1.95) | 0.0295 |
| mg TID (N = 34) | | | | | | | | | |
| Rilzabrutinib 400 | 33 | 15.45 (3.72) | 7.33 (6.55) | −8.18 (0.97) | −2.73 (−5.33, −0.13) | 0.0394 | −52.28 (6.33) | −20.13 (−37.11, −3.16) | 0.0201 |
| mg BID (N = 35) | | | | | | | | | |
| Rilzabrutinib 400 | 33 | 16.38 (3.50) | 10.72 (5.26) | −6.08 (0.95) | −0.63 (−3.22, 1.96) | 0.6343 | −38.14 (6.20) | −5.99 (−22.96, 10.98) | 0.4887 |
| mg QPM (N = 37) | | | | | | | | | |
| Placebo (N = 36) | 33 | 15.14 (4.16) | 10.15 (6.23) | −5.45 (0.97) | | | −32.14 (6.36) | | |
| Week 9 | | | | | | | | | |
| Rilzabrutinib 400 | 29 | 16.15 (3.32) | 6.68 (5.31) | −8.79 (1.02) | −3.67 (−6.38, −0.96) | 0.0079 | −53.45 (6.52) | −23.53 (−40.91, −6.16) | 0.0080 |
| mg TID (N = 34) | | | | | | | | | |
| Rilzabrutinib 400 | 29 | 15.45 (3.72) | 7.63 (7.11) | −7.98 (0.99) | −2.86 (−5.51, −0.21) | 0.0344 | −51.40 (6.37) | −21.48 (−38.50, −4.47) | 0.0133 |
| mg BID (N = 35) | | | | | | | | | |
| Rilzabrutinib 400 | 34 | 16.38 (3.50) | 10.46 (5.15) | −6.16 (0.95) | −1.04 (−3.67, 1.58) | 0.4355 | −37.28 (6.13) | −7.36 (−24.20, 9.48) | 0.3915 |
| mg QPM (N = 37) | | | | | | | | | |
| Placebo (N = 36) | 32 | 15.14 (4.16) | 10.55 (5.94) | −5.12 (0.98) | | | −29.92 (6.31) | | |
| Week 10 | | | | | | | | | |
| Rilzabrutinib 400 | 30 | 16.15 (3.32) | 6.75 (5.25) | −8.88 (1.04) | −3.21 (−5.98, −0.44) | 0.0230 | −54.26 (6.73) | −19.20 (−37.21, −1.20) | 0.0366 |
| mg TID (N = 34) | | | | | | | | | |
| Rilzabrutinib 400 | 28 | 15.45 (3.72) | 8.17 (7.29) | −7.86 (1.04) | −2.19 (−4.95, 0.57) | 0.1198 | −49.04 (6.78) | −13.98 (−31.91, 3.95) | 0.1264 |
| mg BID (N = 35) | | | | | | | | | |
| Rilzabrutinib 400 | 34 | 16.38 (3.50) | 10.43 (5.30) | −6.41 (0.98) | −0.75 (−3.45, 1.96) | 0.5891 | −39.06 (6.40) | −3.99 (−21.57, 13.58) | 0.6560 |
| mg QPM (N = 37) | | | | | | | | | |
| Placebo (N = 36) | 33 | 15.14 (4.16) | 10.00 (6.49) | −5.67 (1.02) | | | −35.06 (6.61) | | |
| Week 11 | | | | | | | | | |
| Rilzabrutinib 400 | 31 | 16.15 (3.32) | 6.99 (5.53) | −8.82 (1.00) | −3.49 (−6.21, −0.78) | 0.0116 | −53.60 (6.61) | −22.12 (−39.99, −4.26) | 0.0152 |
| mg TID (N = 34) | | | | | | | | | |
| Rilzabrutinib 400 | 29 | 15.45 (3.72) | 7.93 (6.94) | −7.71 (1.02) | −2.38 (−5.10, 0.33) | 0.0851 | −49.26 (6.74) | −17.79 (−35.67, 0.10) | 0.0513 |
| mg BID (N = 35) | | | | | | | | | |
| Rilzabrutinib 400 | 33 | 16.38 (3.50) | 11.03 (5.55) | −5.84 (0.98) | −0.51 (−3.21, 2.18) | 0.7077 | −36.51 (6.46) | −5.03 (−22.79, 12.72) | 0.5785 |
| mg QPM (N = 37) | | | | | | | | | |
| Placebo (N = 36) | 33 | 15.14 (4.16) | 10.24 (6.31) | −5.32 (1.00) | | | −31.47 (6.58) | | |
| Week 12 | | | | | | | | | |
| Rilzabrutinib 400 | 28 | 16.15 (3.32) | 5.77 (5.26) | −9.57 (1.06) | −3.86 (−6.71, −1.02) | 0.0078 | −58.20 (6.85) | −23.57 (−41.96, −5.17) | 0.0120 |
| mg TID (N = 34) | | | | | | | | | |
| Rilzabrutinib 400 | 26 | 15.45 (3.72) | 8.09 (6.95) | −7.76 (1.06) | −2.05 (−4.84, 0.74) | 0.1491 | −49.36 (6.89) | −14.72 (−32.82, 3.38) | 0.1109 |
| mg BID (N = 35) | | | | | | | | | |
| Rilzabrutinib 400 | 30 | 16.38 (3.50) | 11.51 (5.54) | −5.29 (1.01) | 0.42 (−2.33, 3.18) | 0.7634 | −34.35 (6.52) | 0.29 (−17.53, 18.11) | 0.9746 |
| mg QPM (N = 37) | | | | | | | | | |
| Placebo (N = 36) | 32 | 15.14 (4.16) | 10.02 (6.82) | −5.71 (1.02) | | | −34.64 (6.62) | | |

^{a}Each of the imputed complete data were analyzed by fitting an ANCOVA model with the corresponding baseline value, intervention group and region as covariates.
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF; other missing data were imputed by MI.
Descriptive statistics at each week include participants with WOCF imputation at the week, and participants whose values were imputed by MI at the week were excluded from the descriptive analysis. One patient was randomized to Rilzabrutinib 400 mg TID group by mistake (baseline ISS7/UAS7 = 0). Removed this participant from the analysis.

A significant proportion of patients in both the ITT and omalizumab-naïve population experienced a MCID (a reduction in ISS7 of at least 5 points) that was sustained over the course of the double-blind treatment phase (see Table 32).

TABLE 32
Proportion of Participants with ISS7 Reduction from Baseline of 5 Points or More Over Time up to Week 12 (ITT
and Omalizumab-Naïve Populations)

| | ITT Population | | | | | | Omalizumab-Naïve Population | | | | | |
|---|---|---|---|---|---|---|---|---|---|---|---|---|
| | | | Imputed | RRD (%) for | | | | | Imputed | RRD (%) | OR (95% CI) | |
| | | | non- | rilzabrutinib | OR (95% CI) | | | | non- | for rilzabrutinib | for | |
| | | Non-responder | responder | vs. placebo | for rilzabrutinib | | | Non-responder | responder | vs. placebo | rilzabrutinib | P-value |
| | Responder n (%) | n (%) | n (%) | (95% CI)^{a} | vs. placeboª | P-value vs. placebo^{b} | Responder n (% ) | n (%) | n (%) | (95% CI)^{a} | vs. placeboª | vs. placebo^{b} |
| Week 1 | | | | | | | | | | | | |
| Rilzabrutinib 400 mg | 27 (65.9) | 14 (34.1) | 0 | 42.3 | 6.428 | 0.0002 | 22 (62.9) | 13 (37.1) | 0 | 40.1 | 5.851 | 0.0009 |
| TID (N = 41) | | | | (22.48, 62.19) | (2.369, 17.441) | | | | | (18.80, 61.44) | (2.026, 16.899) | |
| Rilzabrutinib 400 mg | 20 (48.8) | 21 (51.2) | 1 (2.4) | 25.5 | 3.262 | 0.0180 | 17 (48.6) | 18 (51.4) | 0 | 25.3 | 3.240 | 0.0267 |
| BID (N = 41) | | | | (5.45, 45.48) | (1.222, 8.713) | | | | | (3.89, 46.70) | (1.139, 9.215) | |
| Rilzabrutinib 400 mg | 13 (34.2) | 25 (65.8) | 0 | 12.7 | 1.855 | 0.2221 | 12 (32.4) | 25 (67.6) | 0 | 10.5 | 1.691 | 0.3218 |
| QPM (N = 38) | | | | (−7.46, 32.82) | (0.686, 5.019) | | | | | (−9.99, 31.07) | (0.600, 4.768) | |
| Placebo (N = 40) | 9 (22.5) | 31 (77.5) | 0 | | | | 8 (22.2) | 28 (77.8) | 0 | | | |
| Week 2 | | | | | | | | | | | | |
| Rilzabrutinib 400 mg | 28 (68.3) | 13 (31.7) | 0 | 35.9 | 4.772 | 0.0013 | 23 (65.7) | 12 (34.3) | 0 | 36.4 | 4.876 | 0.0023 |
| TID (N = 41) | | | | (15.40, 56.30) | (1.806, 12.610) | | | | | (14.67, 58.20) | (1.722, 13.807) | |
| Rilzabrutinib 400 mg | 22 (53.7) | 19 (46.3) | 2 (4.9) | 23.6 | 2.693 | 0.0349 | 18 (51.4) | 17 (48.6) | 1 (2.9) | 24.1 | 2.836 | 0.0420 |
| BID (N = 41) | | | | (2.60, 44.66) | (1.073, 6.761) | | | | | (2.00, 46.13) | (1.042, 7.719) | |
| Rilzabrutinib 400 mg | 15 (39.5) | 23 (60.5) | 0 | 11.0 | 1.662 | 0.3076 | 14 (37.8) | 23 (62.2) | 0 | 10.6 | 1.637 | 0.3389 |
| QPM (N = 38) | | | | (−9.68, 31.70) | (0.634, 4.356) | | | | | (−10.65, 31.84) | (0.603, 4.439) | |
| Placebo (N = 40) | 12 (30.0) | 28 (70.0) | 1 (2.5) | | | | 10 (27.8) | 26 (72.2) | 1 (2.8) | | | |
| Week 3 | | | | | | | | | | | | |
| Rilzabrutinib 400 mg | 29 (70.7) | 12 (29.3) | 0 | 37.3 | 4.110 | 0.0011 | 23 (65.7) | 12 (34.3) | 0 | 34.3 | 3.637 | 0.0053 |
| TID (N = 41) | | | | (15.73, 58.95) | (1.671, 10.108) | | | | | (10.93, 57.65) | (1.411, 9.374) | |
| Rilzabrutinib 400 mg | 22 (53.7) | 19 (46.3) | 1 (2.4) | 21.5 | 2.451 | 0.0549 | 18 (51.4) | 17 (48.6) | 0 | 21.1 | 2.418 | 0.0770 |
| BID (N = 41) | | | | (0.36, 42.64) | (0.981, 6.119) | | | | | (−1.44, 43.66) | (0.911, 6.416) | |
| Rilzabrutinib 400 mg | 18 (47.4) | 20 (52.6) | 0 | 15.9 | 1.881 | 0.1588 | 17 (45.9) | 20 (54.1) | 0 | 16.0 | 1.910 | 0.1686 |
| QPM (N = 38) | | | | (−6.26, 38.11) | (0.766, 4.618) | | | | | (−6.65, 38.62) | (0.749, 4.870) | |
| Placebo (N = 40) | 13 (32.5) | 27 (67.5) | 1 (2.5) | | | | 11 (30.6) | 25 (69.4) | 1 (2.8) | | | |
| Week 4 | | | | | | | | | | | | |
| Rilzabrutinib 400 mg | 28 (68.3) | 13 (31.7) | 3 (7.3) | 18.8 | 2.155 | 0.0939 | 24 (68.6) | 11 (31.4) | 3 (8.6) | 25.1 | 2.777 | 0.0384 |
| TID (N = 41) | | | | (−2.90, 40.54) | (0.875, 5.310) | | | | | (2.13, 48.17) | (1.051, 7.333) | |
| Rilzabrutinib 400 mg | 22 (53.7) | 19 (46.3) | 4 (9.8) | 5.6 | 1.252 | 0.6227 | 18 (51.4) | 17 (48.6) | 2 (5.7) | 9.7 | 1.476 | 0.4279 |
| BID (N = 41) | | | | (−16.31, 27.56) | (0.521, 3.011) | | | | | (−13.58, 33.00) | (0.577, 3.779) | |
| Rilzabrutinib 400 mg | 18 (47.4) | 20 (52.6) | 1 (2.6) | 1.7 | 1.076 | 0.8779 | 17 (45.9) | 20 (54.1) | 1 (2.7) | 5.0 | 1.231 | 0.6670 |
| QPM (N = 38) | | | | (−19.99, 23.44) | (0.430, 2.691) | | | | | (−17.54, 27.58) | (0.482, 3.146) | |
| Placebo (N = 40) | 19 (47.5) | 21 (52.5) | 2 (5.0) | | | | 15 (41.7) | 21 (58.3) | 2 (5.6) | | | |
| Week 5 | | | | | | | | | | | | |
| Rilzabrutinib 400 mg | 30 (73.2) | 11 (26.8) | 3 (7.3) | 20.9 | 2.433 | 0.0603 | 25 (71.4) | 10 (28.6) | 2 (5.7) | 22.7 | 2.535 | 0.0599 |
| TID (N = 41) | | | | (−0.26, 42.03) | (0.960, 6.167) | | | | | (−0.15, 45.54) | (0.951, 6.754) | |
| Rilzabrutinib 400 mg | 23 (56.1) | 18 (43.9) | 5 (12.2) | 2.7 | 1.112 | 0.8143 | 20 (57.1) | 15 (42.9) | 2 (5.7) | 7.3 | 1.334 | 0.5478 |
| BID (N = 41) | | | | (−19.28, 24.60) | (0.463, 2.675) | | | | | (−16.20, 30.77) | (0.525, 3.390) | |
| Rilzabrutinib 400 mg | 17 (44.7) | 21 (55.3) | 2 (5.3) | 6.2 | 0.781 | 0.5860 | 16 (43.2) | 21 (56.8) | 2 (5.4) | −6.3 | 0.787 | 0.5972 |
| QPM (N = 38) | | | | (−28.55, 16.17) | (0.321, 1.905) | | | | | (−29.76, 17.25) | (0.320, 1.938) | |
| Placebo (N = 40) | 21 (52.5) | 19 (47.5) | 3 (7.5) | | | | 18 (50.0) | 18 (50.0) | 3 (8.3) | | | |
| Week 6 | | | | | | | | | | | | |
| Rilzabrutinib 400 mg | 25 (61.0) | 16 (39.0) | 5 (12.2) | 11.1 | 1.574 | 0.3257 | 20 (57.1) | 15 (42.9) | 4 (11.4) | 11.3 | 1.563 | 0.3556 |
| TID (N = 41) | | | | (−10.74, 32.99) | (0.643, 3.851) | | | | | (−12.38, 35.07) | (0.611, 3.998) | |
| Rilzabrutinib 400 mg | 21 (51.2) | 20 (48.8) | 4 (9.8) | 2.7 | 1.112 | 0.8133 | 18 (51.4) | 17 (48.6) | 2 (5.7) | 7.1 | 1.326 | 0.5593 |
| BID (N = 41) | | | | (−19.31, 24.65) | (0.463, 2.670) | | | | | (−16.32, 30.44) | (0.520, 3.380) | |
| Rilzabrutinib 400 mg | 18 (47.4) | 20 (52.6) | 4 (10.5) | 1.5 | 1.066 | 0.8938 | 17 (45.9) | 20 (54.1) | 4 (10.8) | 2.0 | 1.088 | 0.8639 |
| QPM (N = 38) | | | | (−20.31, 23.37) | (0.428, 2.654) | | | | | (−20.59, 24.65) | (0.427, 2.773) | |
| Placebo (N = 40) | 19 (47.5) | 21 (52.5) | 6 (15.0) | | | | 16 (44.4) | 20 (55.6) | 5 (13.9) | | | |
| Week 7 | | | | | | | | | | | | |
| Rilzabrutinib 400 mg | 27 (65.9) | 14 (34.1) | 6 (14.6) | 16.8 | 2.038 | 0.1300 | 24 (68.6) | 11 (31.4) | 5 (14.3) | 23.5 | 2.715 | 0.0485 |
| TID (N = 41) | | | | (−4.53, 38.17) | (0.810, 5.128) | | | | | (1.00, 45.99) | (0.995, 7.404) | |
| Rilzabrutinib 400 mg | 22 (53.7) | 19 (46.3) | 4 (9.8) | 5.5 | 1.258 | 0.6214 | 19 (54.3) | 16 (45.7) | 2 (5.7) | 10.2 | 1.533 | 0.3942 |
| BID (N = 41) | | | | (−16.01, 27.10) | (0.515, 3.075) | | | | | (−12.59, 33.02) | (0.586, 4.011) | |
| Rilzabrutinib 400 mg | 19 (50.0) | 19 (50.0) | 6 (15.8) | 3.1 | 1.138 | 0.7854 | 18 (48.6) | 19 (51.4) | 6 (16.2) | 4.0 | 1.176 | 0.7346 |
| QPM (N = 38) | | | | (−18.79, 25.00) | (0.458, 2.826) | | | | | (−18.90, 26.93) | (0.467, 2.962) | |
| Placebo (N = 40) | 19 (47.5) | 21 (52.5) | 5 (12.5) | | | | 16 (44.4) | 20 (55.6) | 4 (11.1) | | | |
| Week 8 | | | | | | | | | | | | |
| Rilzabrutinib 400 mg | 28 (68.3) | 13 (31.7) | 8 (19.5) | 14.6 | 1.839 | 0.1862 | 23 (65.7) | 12 (34.3) | 7 (20.0) | 16.1 | 1.891 | 0.1770 |
| TID (N = 41) | | | | (−6.94, 36.13) | (0.738, 4.585) | | | | | (−7.40, 39.61) | (0.730, 4.901) | |
| Rilzabrutinib 400 mg | 25 (61.0) | 16 (39.0) | 5 (12.2) | 8.3 | 1.388 | 0.4623 | 22 (62.9) | 13 (37.1) | 3 (8.6) | 13.7 | 1.690 | 0.2599 |
| BID (N = 41) | | | | (−13.74, 30.43) | (0.580, 3.321) | | | | | (−10.03, 37.36) | (0.671, 4.258) | |
| Rilzabrutinib 400 mg | 18 (47.4) | 20 (52.6) | 7 (18.4) | −4.5 | 0.836 | 0.6963 | 17 (45.9) | 20 (54.1) | 7 (18.9) | −4.4 | 0.843 | 0.7111 |
| QPM (N = 38) | | | | (−26.75, 17.83) | (0.342, 2.045) | | | | | (−27.59, 18.88) | (0.339, 2.100) | |
| Placebo (N = 40) | 21 (52.5) | 19 (47.5) | 5 (12.5) | | | | 18 (50.0) | 18 (50.0) | 4 (11.1) | | | |
| Week 9 | | | | | | | | | | | | |
| Rilzabrutinib 400 mg | 29 (70.7) | 12 (29.3) | 7 (17.1) | 22.5 | 2.730 | 0.0383 | 24 (68.6) | 11 (31.4) | 6 (17.1) | 25.1 | 2.956 | 0.0315 |
| TID (N = 41) | | | | (1.82, 43.14) | (1.042, 7.150) | | | | | (2.93, 47.37) | (1.064, 8.208) | |
| Rilzabrutinib 400 mg | 22 (53.7) | 19 (46.3) | 9 (22.0) | 5.6 | 1.233 | 0.6227 | 19 (54.3) | 16 (45.7) | 7 (20.0) | 10.4 | 1.462 | 0.3922 |
| BID (N = 41) | | | | (−17.08, 28.33) | (0.528, 2.880) | | | | | (−13.98, 34.88) | (0.596, 3.586) | |
| Rilzabrutinib 400 mg | 20 (52.6) | 18 (47.4) | 6 (15.8) | 5.8 | 1.294 | 0.5965 | 19 (51.4) | 18 (48.6) | 6 (16.2) | 6.5 | 1.328 | 0.5653 |
| QPM (N = 38) | | | | (−15.33, 26.96) | (0.505, 3.321) | | | | | (−15.49, 28.41) | (0.506, 3.483) | |
| Placebo (N = 40) | 19 (47.5) | 21 (52.5) | 6 (15.0) | | | | 16 (44.4) | 20 (55.6) | 5 (13.9) | | | |
| Week 10 | | | | | | | | | | | | |
| Rilzabrutinib 400 mg | 27 (65.9) | 14 (34.1) | 7 (17.1) | 11.9 | 1.669 | 0.2796 | 23 (65.7) | 12 (34.3) | 5 (14.3) | 15.5 | 1.910 | 0.1910 |
| TID (N = 41) | | | | (−9.36, 33.15) | (0.662, 4.207) | | | | | (−7.42, 38.40) | (0.719, 5.077) | |
| Rilzabrutinib 400 mg | 19 (46.3) | 22 (53.7) | 10 (24.4) | −6.2 | 0.786 | 0.5921 | 16 (45.7) | 19 (54.3) | 8 (22.9) | −3.1 | 0.883 | 0.7963 |
| BID (N = 41) | | | | (−28.29, 15.99) | (0.329, 1.875) | | | | | (−26.73, 20.43) | (0.349, 2.235) | |
| Rilzabrutinib 400 mg | 24 (63.2) | 14 (36.8) | 6 (15.8) | 11.2 | 1.593 | 0.3280 | 23 (62.2) | 14 (37.8) | 6 (16.2) | 11.9 | 1.614 | 0.3157 |
| QPM (N = 38) | | | | (−10.58, 32.94) | (0.637, 3.986) | | | | | (−10.94, 34.75) | (0.639, 4.080) | |
| Placebo (N = 40) | 21 (52.5) | 19 (47.5) | 5 (12.5) | | | | 18 (50.0) | 18 (50.0) | 4 (11.1) | | | |
| Week 11 | | | | | | | | | | | | |
| Rilzabrutinib 400 mg | 29 (70.7) | 12 (29.3) | 6 (14.6) | 11.8 | 1.699 | 0.2734 | 25 (71.4) | 10 (28.6) | 4 (11.4) | 15.5 | 1.951 | 0.1834 |
| TID (N = 41) | | | | (−9.03, 32.68) | (0.661, 4.362) | | | | | (−7.09, 38.00) | (0.720, 5.283) | |
| Rilzabrutinib 400 mg | 22 (53.7) | 19 (46.3) | 9 (22.0) | −4.5 | 0.837 | 0.6935 | 19 (54.3) | 16 (45.7) | 7 (20.0) | −1.0 | 0.963 | 0.9376 |
| BID (N = 41) | | | | (−26.51, 17.54) | (0.349, 2.007) | | | | | (−24.66, 22.75) | (0.383, 2.426) | |
| Rilzabrutinib 400 mg | 21 (55.3) | 17 (44.7) | 7 (18.4) | −1.5 | 0.938 | 0.8934 | 20 (54.1) | 17 (45.9) | 7 (18.9) | −1.7 | 0.932 | 0.8852 |
| QPM (N = 38) | | | | (−23.17, 20.15) | (0.374, 2.353) | | | | | (−24.23, 20.85) | (0.364, 2.385) | |
| Placebo (N = 40) | 23 (57.5) | 17 (42.5) | 5 (12.5) | | | | 20 (55.6) | 16 (44.4) | 4 (11.1) | | | |
| Week 12 | | | | | | | | | | | | |
| Rilzabrutinib 400 mg | 23 (56.1) | 18 (43.9) | 11 (26.8) | 7.9 | 1.371 | 0.4839 | 20 (57.1) | 15 (42.9) | 8 (22.9) | 9.7 | 1.469 | 0.4230 |
| TID (N = 41) | | | | (−14.19, 29.98) | (0.566, 3.320) | | | | | (−13.92, 33.30) | (0.572, 3.772) | |
| Rilzabrutinib 400 mg | 20 (48.8) | 21 (51.2) | 14 (34.1) | 1.6 | 1.063 | 0.8886 | 17 (48.6) | 18 (51.4) | 11 (31.4) | 2.4 | 1.095 | 0.8444 |
| BID (N = 41) | | | | (−20.93, 24.12) | (0.452, 2.497) | | | | | (−21.62, 26.39) | (0.440, 2.724) | |
| Rilzabrutinib 400 mg | 17 (44.7) | 21 (55.3) | 10 (26.3) | −2.6 | 0.905 | 0.8245 | 16 (43.2) | 21 (56.8) | 10 (27.0) | −4.2 | 0.851 | 0.7237 |
| QPM (N = 38) | | | | (−25.28, 20.14) | (0.377, 2.177) | | | | | (−27.77, 19.30) | (0.346, 2.092) | |
| Placebo (N = 40) | 19 (47.5) | 21 (52.5) | 7 (17.5) | | | | 17 (47.2) | 19 (52.8) | 5 (13.9) | | | |

^{b}CMH test was performed on the association between the responder status and intervention group, stratified by region.
Note:
Participants who received selected prohibited/rescue medications (high/medium impact on efficacy confirmed through blinded medical review) before the given week were considered as non-responders, and participants with missing data at the given week were considered as non-responders up to Week 12.

### HSS7

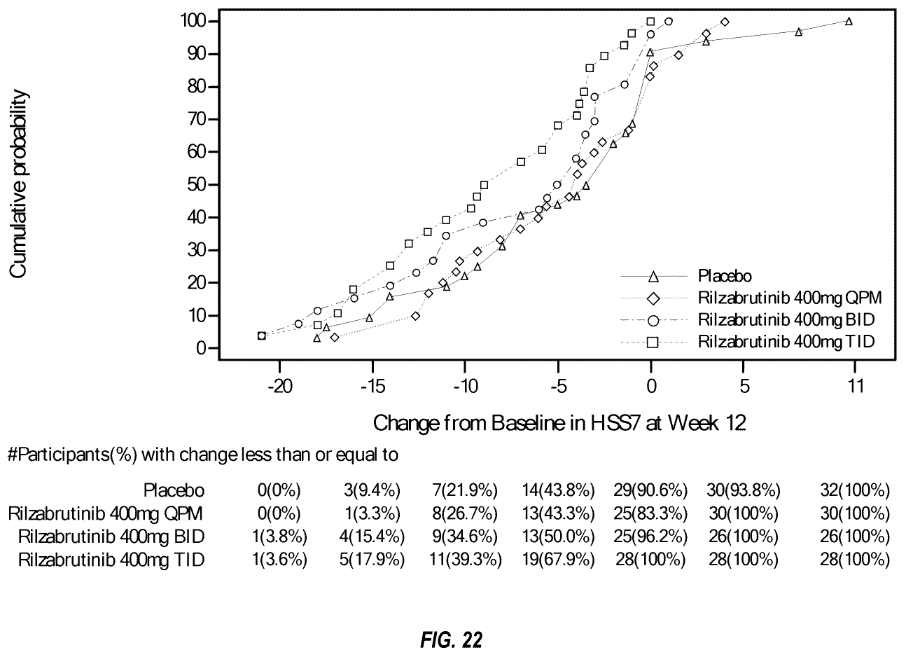

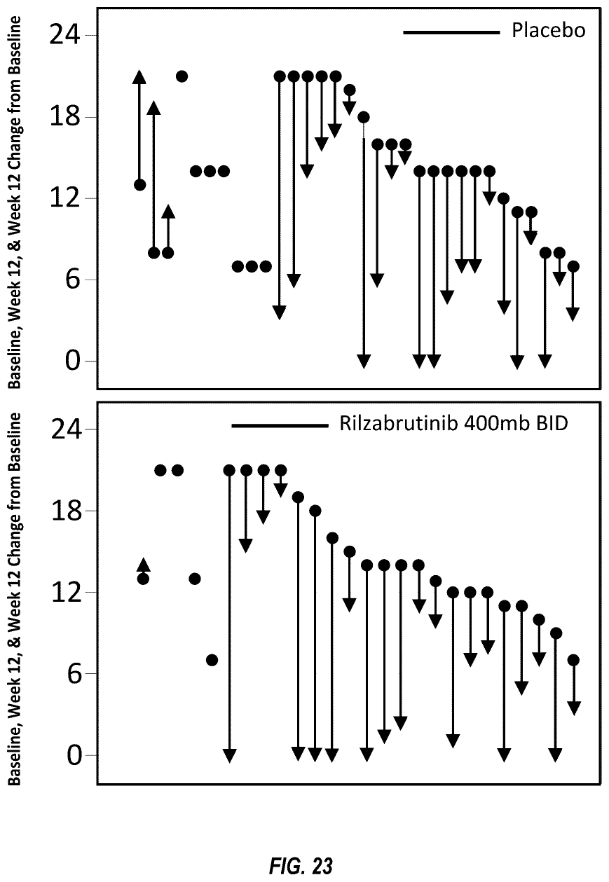

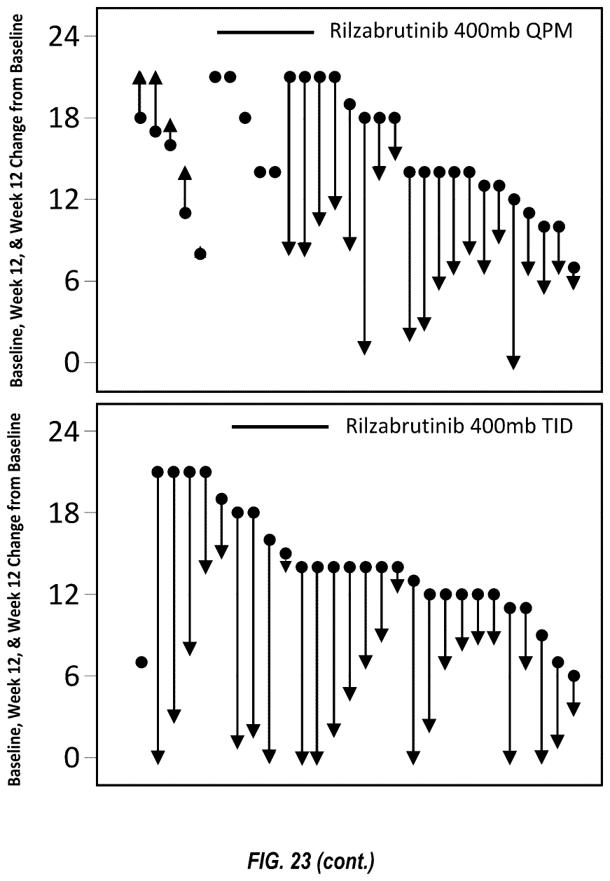

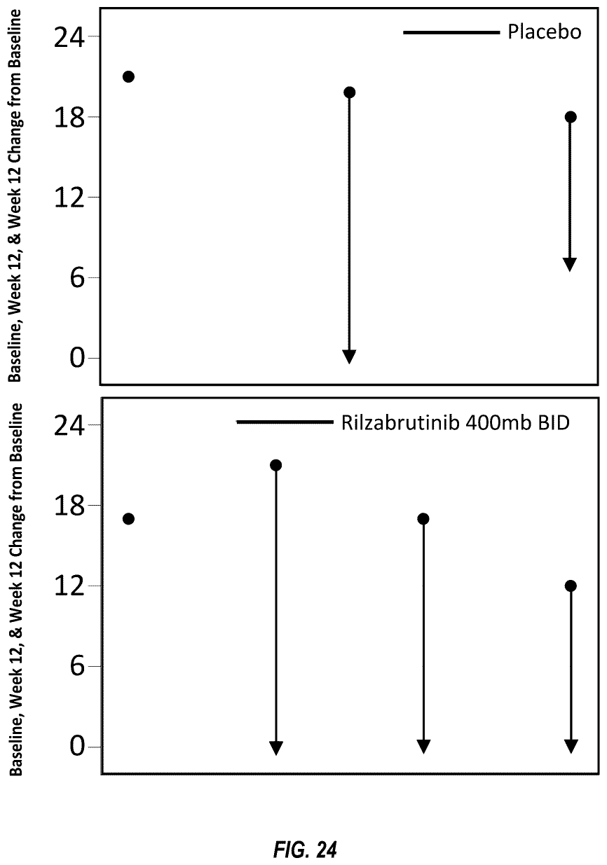

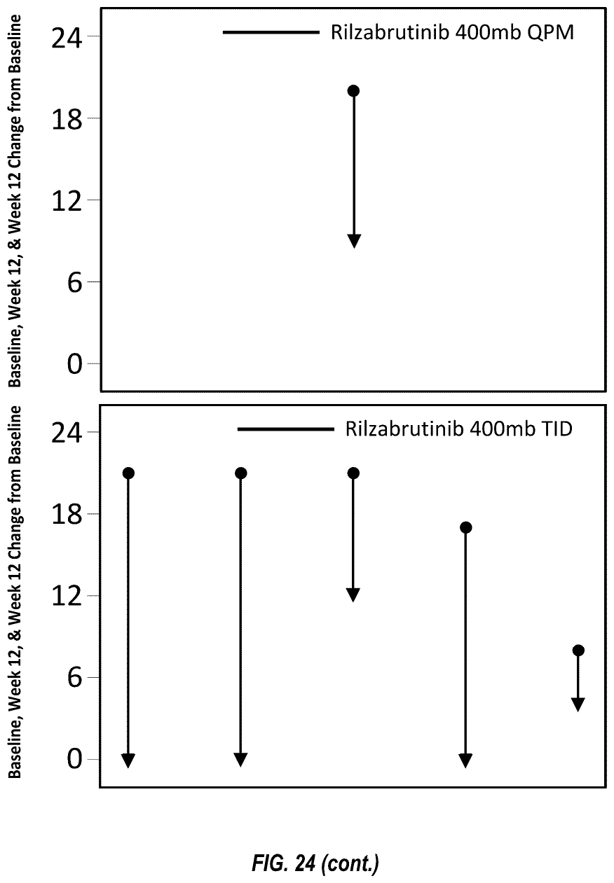

Rilzabrutinib treatment led to significant improvements in HSS7. Notably, participants receiving 400 mg TID rilzabrutinib exhibited a significant reduction in HSS7 at Week 12 (see FIGS. 22-24 and Tables 33-35). In the ITT population, participants receiving 400 mg TID rilzabrutinib exhibited a median reduction in HSS7 of −8.31 (p=0.0126) points. Meanwhile, patients receiving the placebo exhibited a median reduction in HSS7 of −4.89 points (see Table 33). Similar results were observed for the omalizumab-naïve population, both prior to (median reduction of −7.64, p=0.0257; see Table 34) and following (median reduction of −7.89, p=0.0156; see Table 35) the removal of the asymptomatic outlier (UAS7/ISS7=0 at baseline) who was erroneously randomized into the 400 mg TID rilzabrutinib arm. Notably, in the 400 mg TID arm, all patients except one showed improvement in ISS7.

TABLE 33
Supplementary analysis: Change from baseline in HSS7 at Week 12 (ITT Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| HSS7 | (N = 40) | (N = 38) | (N = 41) | (N = 41) |

Baseline

| Number | 40 | | 38 | | 41 | | 41 | |
|---|---|---|---|---|---|---|---|---|
| Mean (SD) | 14.37 | (4.89) | 14.87 | (4.29) | 14.41 | (4.26) | 14.00 | (4.77) |

| | | | | |
|---|---|---|---|---|
| Median | 14.00 | 14.00 | 14.00 | 14.00 |
| Q1; Q3 | 11.00; 18.92 | 12.00; 18.00 | 12.00; 17.00 | 12.00; 17.00 |
| Min; Max | 7.0; 21.0 | 7.0; 21.0 | 7.0; 21.0 | 0.0; 21.0 |

Week 12

| | | | | | | | | |
|---|---|---|---|---|---|---|---|---|
| Number (observed/imputed) | 35 | (33/2) | 31 | (28/3) | 30 | (27/3) | 33 | (30/3) |
| Mean (SD) | 9.31 | (6.74) | 10.47 | (6.04) | 7.08 | (7.32) | 4.92 | (4.89) |

| | | | | |
|---|---|---|---|---|
| Median | 7.00 | 8.75 | 6.00 | 3.50 |
| Q1; Q3 | 4.00; 14.00 | 7.00; 14.00 | 0.00; 13.00 | 0.00; 8.40 |
| Min; Max | 0.0; 21.0 | 0.0; 21.0 | 0.0; 21.0 | 0.0; 15.2 |

Change from baseline

| Number (observed/imputed) | 35 | (33/2) | 31 | (28/3) | 30 | (27/3) | 33 | (30/3) |
|---|---|---|---|---|---|---|---|---|
| Mean (SD) | −5.09 | (7.08) | −5.05 | (5.64) | −7.81 | (7.00) | −9.62 | (6.34) |
| Median | −3.50 | | −4.00 | | −5.30 | | −9.00 | |
| Q1; Q3 | −10.00; 0.00 | | −10.50; 0.00 | | −12.60; −3.00 | | −14.00; −4.00 | |
| Min; Max | −19.8; 10.7 | | −17.0; 4.0 | | −21.0; 1.0 | | −21.0; 0.0 | |
| LS Mean (SE) ^{a} | −4.89 | (1.02) | −4.75 | (1.03) | −6.78 | (1.02) | −8.31 | (1.00) |
| LS Mean Diff vs. placebo (95% CI) ^{a} | | | 0.14 | (−2.62, 2.91) | −1.89 | (−4.58, 0.80) | −3.42 | (−6.11, −0.74) |

| | | | |
|---|---|---|---|
| P-value vs. placebo ^{a} | 0.9186 | 0.1683 | 0.0126 |

^{a} Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.

TABLE 34
Change from Baseline in HSS7 at Week 12 (Omalizumab-naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| HSS7 | (N = 36) | (N = 37) | (N = 35) | (N = 35) |

Baseline

| Number | 36 | | 37 | | 35 | | 35 | |
|---|---|---|---|---|---|---|---|---|
| Mean (SD) | 13.83 | (4.85) | 14.73 | (4.26) | 14.28 | (4.21) | 13.66 | (4.47) |

| | | | | |
|---|---|---|---|---|
| Median | 14.00 | 14.00 | 14.00 | 14.00 |
| Q1; Q3 | 9.50; 17.00 | 12.00; 18.00 | 12.00; 18.00 | 12.00; 16.00 |
| Min; Max | 7.0; 21.0 | 7.0; 21.0 | 7.0; 21.0 | 0.0; 21.0 |

Week 12

| | | | | | | | | |
|---|---|---|---|---|---|---|---|---|
| Number (observed/imputed) | 32 | (31/1) | 30 | (27/3) | 26 | (24/2) | 28 | (27/1) |
| Mean (SD) | 9.31 | (6.52) | 10.52 | (6.14) | 7.52 | (7.21) | 5.23 | (4.86) |

| | | | | |
|---|---|---|---|---|
| Median | 7.00 | 8.58 | 7.00 | 4.08 |
| Q1; Q3 | 4.33; 14.00 | 7.00; 14.00 | 0.00; 13.00 | 0.58; 8.58 |
| Min; Max | 0.0; 21.0 | 0.0; 21.0 | 0.0; 21.0 | 0.0; 15.2 |

Change from baseline

| Number (observed/imputed) | 32 | (31/1) | 30 | (27/3) | 26 | (24/2) | 28 | (27/1) |
|---|---|---|---|---|---|---|---|---|
| Mean (SD) | −4.60 | (6.76) | −4.85 | (5.63) | −7.09 | (6.54) | −8.77 | (5.84) |
| Median | −2.75 | | −4.00 | | −4.50 | | −8.00 | |
| Q1; Q3 | −8.67; 0.00 | | −10.25; 0.00 | | −11.67; −3.00 | | −13.50; −3.72 | |
| Min; Max | −18.0; 10.7 | | −17.0; 4.0 | | −21.0; 1.0 | | −21.0; 0.0 | |
| LS Mean (SE) ^{a} | −4.40 | (1.06) | −4.49 | (1.04) | −6.52 | (1.07) | −7.64 | (1.06) |
| LS Mean Diff vs. placebo (95% CI) ^{a} | | | −0.09 | (−2.92, 2.75) | −2.12 | (−4.97, 0.73) | −3.24 | (−6.08, −0.39) |

| | | | |
|---|---|---|---|
| P-value vs. placebo ^{a} | 0.9531 | 0.1450 | 0.0257 |

^{a} Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values wereimputed by MI at Week 12 were excluded from the descriptive analysis.

TABLE 35
Change from Baseline in HSS7 at Week 12 (Omalizumab-Naïve Population with Outlier Removed from Analysis)

| | | | | |
|---|---|---|---|---|
| | | Rilzabrutinib | Rilzabrutinib | Rilzabrutinib |
| | Placebo | 400 mg QPM | 400 mg BID | 400 mg TID |
| HSS7 | (N = 36) | (N = 37) | (N = 35) | (N = 34) |

Baseline

| Number | 36 | | 37 | | 35 | | 34 | |
|---|---|---|---|---|---|---|---|---|
| Mean (SD) | 13.83 | (4.85) | 14.73 | (4.26) | 14.28 | (4.21) | 14.06 | (3.85) |

| | | | | |
|---|---|---|---|---|
| Median | 14.00 | 14.00 | 14.00 | 14.00 |
| Q1; Q3 | 9.50; 17.00 | 12.00; 18.00 | 12.00; 18.00 | 12.00; 16.00 |
| Min; Max | 7.0; 21.0 | 7.0; 21.0 | 7.0; 21.0 | 6.0; 21.0 |

Week 12

| | | | | | | | | |
|---|---|---|---|---|---|---|---|---|
| Number (observed/imputed) | 32 | (31/1) | 30 | (27/3) | 26 | (24/2) | 28 | (27/1) |
| Mean (SD) | 9.31 | (6.52) | 10.52 | (6.14) | 7.52 | (7.21) | 5.23 | (4.86) |

| | | | | |
|---|---|---|---|---|
| Median | 7.00 | 8.58 | 7.00 | 4.08 |
| Q1; Q3 | 4.33; 14.00 | 7.00; 14.00 | 0.00; 13.00 | 0.58; 8.58 |
| Min; Max | 0.0; 21.0 | 0.0; 21.0 | 0.0; 21.0 | 0.0; 15.2 |

Change from baseline

| Number (observed/imputed) | 32 | (31/1) | 30 | (27/3) | 26 | (24/2) | 28 | (27/1) |
|---|---|---|---|---|---|---|---|---|
| Mean (SD) | −4.60 | (6.76) | −4.85 | (5.63) | −7.09 | (6.54) | −8.77 | (5.84) |
| Median | −2.75 | | −4.00 | | −4.50 | | −8.00 | |
| Q1; Q3 | −8.67; 0.00 | | −10.25; 0.00 | | −11.67; −3.00 | | −13.50; −3.72 | |
| Min; Max | −18.0; 10.7 | | −17.0; 4.0 | | −21.0; 1.0 | | −21.0; 0.0 | |
| LS Mean (SE) ^{a} | −4.35 | (1.04) | −4.57 | (1.02) | −6.56 | (1.07) | −7.89 | (1.07) |
| LS Mean Diff vs. placebo (95% CI) ^{a} | | | −0.22 | (−3.01, 2.57) | −2.20 | (−5.04, 0.63) | −3.54 | (−6.40, −0.67) |

| | | | |
|---|---|---|---|
| P-value vs. placebo ^{a} | 0.8768 | 0.1279 | 0.0156 |

^{a} Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF.
Missing data after study intervention discontinuation for lack of efficacy were imputed by WOCF; other missing data were imputed by MI.
Descriptive statistics at Week 12 include participants with WOCF imputation at Week 12, and participants whose values were imputed by MI at Week 12 were excluded from the descriptive analysis.
One participant was randomized to Rilzabrutinib 400 mg TID group by mistake (baseline ISS7/UAS7 = 0). Removed this participant from the analysis.

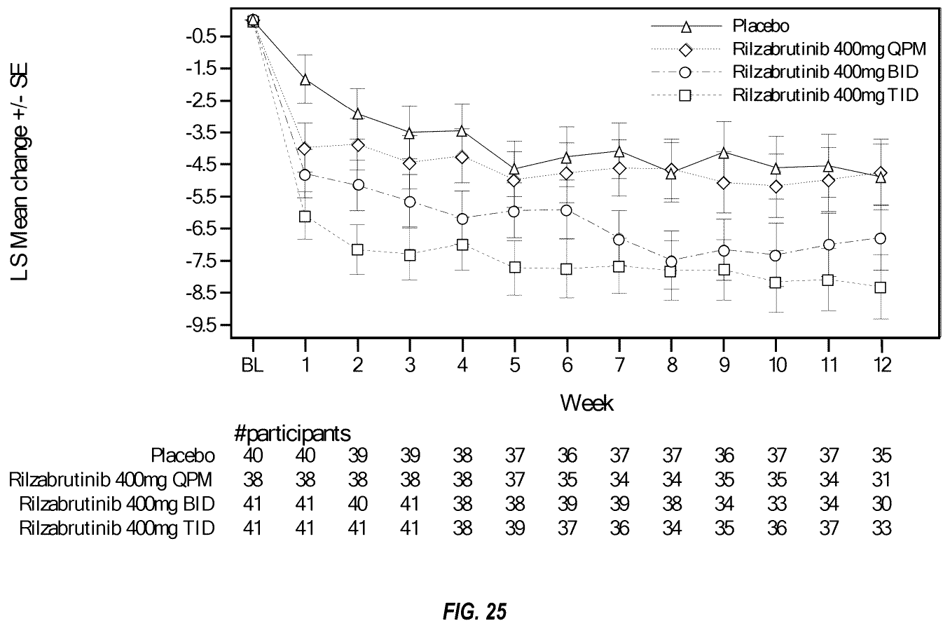

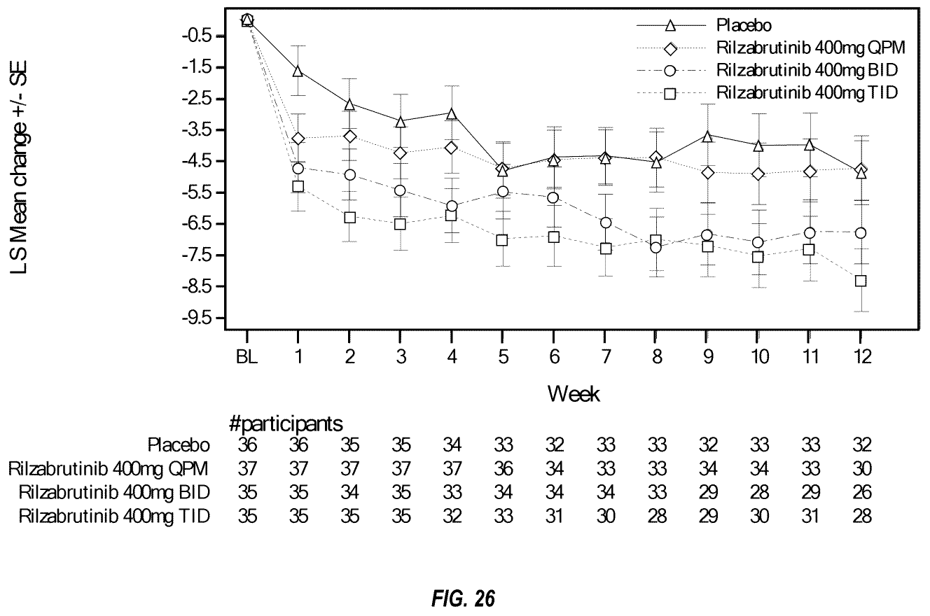

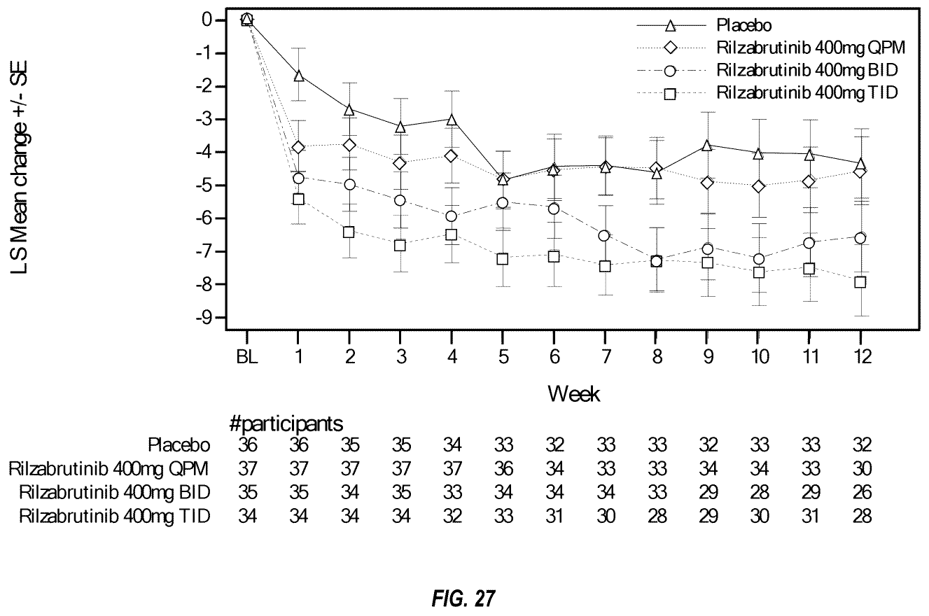

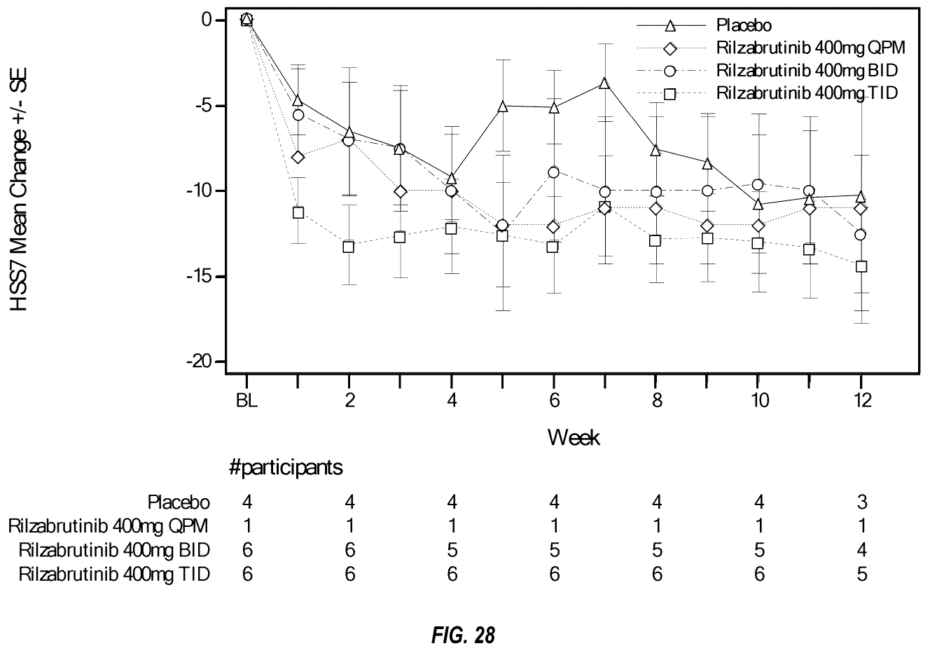

Participants exhibited a rapid and sustained improvement in hives symptoms, with significant reductions in HSS7 observed following one week of treatment (see FIGS. 25-28 and Tables 36-39).

TABLE 36
Summary of HSS7 by Week in the Double-Blind Period (Omalizumab-Naïve Population and Patients with Prior
Omalizumab Use, Including Omalizumab-Incomplete Responders)

| | Omalizumab-Naïve Population | | | | | | | |
|---|---|---|---|---|---|---|---|---|
| | | Rilzabrutinib | Rilzabrutinib | Rilzabrutinib | | Rilzabrutinib | Rilzabrutinib | Rilzabrutinib |
| | Placebo | 400 mg QPM | 400 mg BID | 400 mg TID | Placebo | 400 mg QPM | 400 mg BID | 400 mg TID |
| HSS7 | (N = 36) | (N = 37) | (N = 35) | (N = 35) | (N = 4) | (N = 1) | (N = 6) | (N = 6) |

Baseline

| | | | | | | | | |
|---|---|---|---|---|---|---|---|---|
| Number | 36 | 37 | 35 | 35 | 4 | 1 | 6 | 6 |
| Mean (SD) | 13.83 (4.85) | 14.73 (4.26) | 14.28 (4.21) | 13.66 (4.47) | 19.21 (1.47) | 20.00 (NC) | 15.17 (4.92) | 16.03 (6.35) |
| Median | 14.00 | 14.00 | 14.00 | 14.00 | 18.92 | 20.00 | 17.00 | 19.00 |
| Q1; Q3 | 9.50; 17.00 | 12.00; 18.00 | 12.00; 18.00 | 12.00; 16.00 | 18.00; 20.42 | 20.00; 20.00 | 12.00; 17.00 | 8.17; 21.00 |
| Min; Max | 7.0; 21.0 | 7.0; 21.0 | 7.0; 21.0 | 0.0; 21.0 | 18.0; 21.0 | 20.0; 20.0 | 7.0; 21.0 | 8.0; 21.0 |

Week 1

| | | | | | | | | |
|---|---|---|---|---|---|---|---|---|
| Number | 36 (36/0) | 37 (37/0) | 35 (35/0) | 35 (35/0) | 4 (4/0) | 1 (1/0) | 6 (5/1) | 6 (6/0) |
| (observed/ | | | | | | | | |
| imputed) | | | | | | | | |
| Mean (SD) | 12.23 (5.49) | 10.73 (4.90) | 9.26 (5.76) | 8.26 (4.91) | 14.50 (5.32) | 12.00 (NC) | 9.67 (6.41) | 4.83 (3.31) |
| Median | 12.50 | 10.00 | 10.00 | 8.00 | 14.50 | 12.00 | 10.50 | 4.50 |
| Q1; Q3 | 8.00; 16.00 | 8.00; 14.00 | 4.00; 14.00 | 4.00; 13.00 | 11.00; 18.00 | 12.00; 12.00 | 6.00; 14.00 | 2.00; 7.00 |
| Min; Max | 0.0; 21.0 | 1.0; 21.0 | 0.0; 21.0 | 0.0; 16.0 | 8.0; 21.0 | 12.0; 12.0 | 0.0; 17.0 | 1.0; 10.0 |
| Change from | | | | | | | | |
| baseline | | | | | | | | |
| Number | 36 (36/0) | 37 (37/0) | 35 (35/0) | 35 (35/0) | 4 (4/0) | 1 (1/0) | 6 (5/1) | 6 (6/0) |
| (observed/ | | | | | | | | |
| imputed) | | | | | | | | |
| Mean (SD) | −1.60 (4.68) | −4.00 (4.92) | −5.02 (4.53) | −5.40 (5.44) | −4.71 (4.11) | −8.00 (NC) | −5.50 (6.41) | −11.19 (4.74) |
| Median | −1.00 | −3.00 | −5.00 | −5.00 | −4.42 | −8.00 | −3.00 | −12.50 |
| Q1; Q3 | −3.50; 1.00 | −6.00; 0.00 | −9.00; 0.00 | −9.00; 0.00 | −7.42; −2.00 | −8.00; −8.00 | 12.00; 0.00 | −15.00; −7.00 |
| Min; Max | −15.0; 9.0 | −18.0; 2.0 | −15.0; 1.0 | −19.0; 2.0 | −10.0; 0.0 | 8.0; −8.0 | −15.0; 0.0 | −16.0; −4.2 |
| Percent change | | | | | | | | |
| from baseline | | | | | | | | |
| Number | 36 (36/0) | 37 (37/0) | 35 (35/0) | 34 (34/0) | 4 (4/0) | 1 (1/0) | 6 (5/1) | 6 (6/0) |
| (observed/ | | | | | | | | |
| imputed) | | | | | | | | |
| Mean (SD) | −8.04 (38.95) | −25.31 (29.00) | −36.53 (34.65) | −36.74 (35.15) | −25.54 (22.84) | −40.00 (NC) | −34.45 (41.48) | −70.33 (16.48) |
| Median | −7.74 | −20.00 | −28.57 | −33.33 | −23.30 | −40.00 | −17.65 | −71.43 |
| Q1; Q3 | −26.79; 6.70 | −44.44; 0.00 | −52.38; 0.00 | −61.11; 0.00 | −39.96; −11.11 | −40.00; −40.00 | −71.43; 0.00 | −87.50; −52.38 |
| Min; Max | −100.0; 85.7 | −93.3; 15.4 | −100.0; 14.3 | −100.0; 16.7 | −55.6; 0.0 | −40.0; −40.0 | −100.0; 0.0 | −88.2; −51.0 |

Week 2

| | | | | | | | | |
|---|---|---|---|---|---|---|---|---|
| Number | 35 (35/0) | 37 (37/0) | 34 (34/0) | 35 (35/0) | 4 (4/0) | 1 (1/0) | 6 (5/1) | 6 (6/0) |
| (observed/ | | | | | | | | |
| imputed) | | | | | | | | |
| Mean (SD) | 11.11 (5.32) | 10.90 (4.97) | 9.27 (5.49) | 7.25 (5.05) | 12.67 (8.67) | 13.00 (NC) | 8.17 (7.08) | 2.83 (3.31) |
| Median | 12.00 | 10.00 | 7.50 | 7.00 | 14.33 | 13.00 | 9.50 | 1.50 |
| Q1; Q3 | 8.00; 14.00 | 8.00; 14.00 | 5.00; 14.00 | 4.00; 11.00 | 6.33; 19.00 | 13.00; 13.00 | 0.00; 13.00 | 0.00; 7.00 |
| Min; Max | 0.0; 21.0 | 1.0; 21.0 | 0.0; 21.0 | 0.0; 17.0 | 1.0; 21.0 | 13.0; 13.0 | 0.0; 17.0 | 0.0; 7.0 |
| Change from | | | | | | | | |
| baseline | | | | | | | | |
| Number | 35 (35/0) | 37 (37/0) | 34 (34/0) | 35 (35/0) | 4 (4/0) | 1 (1/0) | 6 (5/1) | 6 (6/0) |
| (observed/ | | | | | | | | |
| imputed) | | | | | | | | |
| Mean (SD) | −2.52 (4.98) | −3.83 (4.91) | −5.10 (4.74) | −6.40 (5.94) | −6.54 (7.44) | −7.00 (NC) | −7.00 (8.15) | −13.19 (5.74) |
| Median | −1.00 | 3.00 | 5.50 | −6.00 | −4.58 | −7.00 | −4.50 | −14.00 |
| Q1; Q3 | −5.00; 1.00 | −7.00; 0.00 | −7.00; 0.00 | −11.00; −1.17 | −11.67; −1.42 | −7.00; −7.00 | −12.00; 0.00 | −17.00; −7.17 |
| Min; Max | −17.0; 7.0 | −16.0; 3.0 | −16.0; 3.6 | −18.0; 2.3 | −17.0; 0.0 | −7.0; −7.0 | −21.0; 0.0 | −21.0; −6.0 |
| Percent change | | | | | | | | |
| from baseline | | | | | | | | |
| Number | 35 (35/0) | 37 (37/0) | 34 (34/0) | 34 (34/0) | 4 (4/0) | 1 (1/0) | 6 (5/1) | 6 (6/0) |
| (observed/ | | | | | | | | |
| imputed) | | | | | | | | |
| Mean (SD) | −14.59 (39.41) | −23.50 (30.60) | −35.29 (34.55) | −44.30 (37.78) | −35.98 (41.57) | −35.00 (NC) | −42.16 (46.38) | 82.68 (15.48) |
| Median | −12.50 | −21.43 | −37.50 | −42.86 | −24.74 | −35.00 | −26.47 | −81.38 |
| Q1; Q3 | −37.50; 6.25 | −44.44; 0.00 | −54.55; 0.00 | −80.95; −10.00 | −64.81; −7.14 | −35.00; −35.00 | −100.00; 0.00 | −100.00; −66.67 |
| Min; Max | −100.0; 100.0 | −93.3; 33.3 | −100.0; 40.0 | −100.0; 16.7 | −94.4; 0.0 | −35.0; −35.0 | −100.0; 0.0 | −100.0; −66.7 |

Week 3

| | | | | | | | | |
|---|---|---|---|---|---|---|---|---|
| Number | 35 (35/0) | 37 (37/0) | 35 (35/0) | 35 (35/0) | 4 (4/0) | 1 (1/0) | 6 (5/1) | 6 (6/0) |
| (observed/ | | | | | | | | |
| imputed) | | | | | | | | |
| Mean (SD) | 10.62 (5.91) | 10.41 (4.97) | 8.55 (6.09) | 7.03 (4.82) | 11.67 (8.08) | 10.00 (NC) | 7.67 (7.00) | 3.39 (2.63) |
| Median | 11.00 | 9.00 | 7.00 | 7.00 | 14.00 | 10.00 | 7.50 | 2.67 |
| Q1; Q3 | 7.00; 14.00 | 7.00; 14.00 | 3.50; 13.00 | 4.00; 11.00 | 7.00; 16.33 | 10.00; 10.00 | 0.00; 14.00 | 2.00; 6.00 |
| Min; Max | 0.0; 21.0 | 0.0; 21.0 | 0.0; 21.0 | 0.0; 17.0 | 0.0; 18.7 | 10.0; 10.0 | 0.0; 17.0 | 0.0; 7.0 |
| Change from | | | | | | | | |
| baseline | | | | | | | | |
| Number | 35 (35/0) | 37 (37/0) | 35 (35/0) | 35 (35/0) | 4 (4/0) | 1 (1/0) | 6 (5/1) | 6 (6/0) |
| (observed/ | | | | | | | | |
| imputed) | | | | | | | | |
| Mean (SD) | −3.01 (5.52) | −4.32 (4.60) | −5.73 (5.31) | −6.63 (6.22) | −7.54 (7.37) | −10.00 (NC) | −7.50 (8.22) | −12.64 (6.13) |
| Median | −2.00 | −2.00 | −5.00 | −7.00 | −5.50 | −10.00 | −6.00 | −14.50 |
| Q1; Q3 | −6.00; 0.00 | −7.00; −0.83 | −9.00; −2.00 | −11.00; −2.00 | −12.50; −2.58 | −10.00; −10.00 | 12.00; 0.00 | −15.00; −5.83 |
| Min; Max | −17.0; 7.0 | −15.0; 3.0 | −20.0; 2.2 | −18.0; 7.0 | −18.0; −1.2 | −10.0; −10.0 | −21.0; 0.0 | −21.0; −5.0 |
| Percent change | | | | | | | | |
| from baseline | | | | | | | | |
| Number | 35 (35/0) | 37 (37/0) | 35 (35/0) | 34 (34/0) | 4 (4/0) | 1 (1/0) | 6 (5/1) | 6 (6/0) |
| (observed/ | | | | | | | | |
| imputed) | | | | | | | | |
| Mean (SD) | −17.74 (46.09) | −27.98 (28.68) | −40.65 (36.39) | −47.46 (36.18) | −40.36 (41.33) | −50.00 (NC) | −45.10 (46.71) | −76.71 (14.38) |
| Median | −12.50 | −28.57 | −41.67 | −48.81 | −27.78 | −50.00 | −35.29 | −71.43 |
| Q1; Q3 | −46.15; 0.00 | −50.00; −6.25 | −66.67; −9.52 | −75.00; −19.05 | −66.67; −14.05 | 50.00; −50.00 | −100.00; 0.00 | −88.24; −66.67 |
| Min; Max | −100.0; 100.0 | −100.0; 37.5 | −100.0; 24.4 | −100.0; 33.3 | −100.0; −5.9 | −50.0; −50.0 | −100.0; 0.0 | −100.0; −62.5 |

Week 4

| | | | | | | | | |
|---|---|---|---|---|---|---|---|---|
| Number | 34 (34/0) | 37 (36/1) | 33 (33/0) | 32 (32/0) | 4 (4/0) | 1 (1/0) | 5 (4/1) | 6 (6/0) |
| (observed/ | | | | | | | | |
| imputed) | | | | | | | | |
| Mean (SD) | 10.58 (6.36) | 10.53 (4.74) | 7.60 (6.00) | 6.85 (4.96) | 9.99 (5.80) | 10.00 (NC) | 6.80 (8.04) | 3.92 (4.26) |
| Median | 12.00 | 10.00 | 7.00 | 6.50 | 9.98 | 10.00 | 2.00 | 3.17 |
| Q1; Q3 | 7.00; 14.00 | 8.00; 14.00 | 2.00; 11.00 | 3.50; 8.08 | 5.88; 14.10 | 10.00; 10.00 | 1.00; 14.00 | 1.17; 4.00 |
| Min; Max | 0.0; 21.0 | 0.0; 21.0 | 0.0; 21.0 | 0.0; 19.0 | 3.0; 17.0 | 10.0; 10.0 | 0.0; 17.0 | 0.0; 12.0 |
| Change from | | | | | | | | |
| baseline | | | | | | | | |
| Number | 34 (34/0) | 37 (36/1) | 33 (33/0) | 32 (32/0) | 4 (4/0) | 1 (1/0) | 5 (4/1) | 6 (6/0) |
| (observed/ | | | | | | | | |
| imputed) | | | | | | | | |
| Mean (SD) | −3.10 (6.11) | −4.20 (4.74) | −6.54 (5.34) | −7.21 (5.24) | −9.22 (4.98) | −10.00 (NC) | −10.00 (8.34) | −12.11 (6.81) |
| Median | −0.50 | −3.00 | −6.00 | −6.58 | −9.53 | −10.00 | −12.00 | −12.42 |
| Q1; Q3 | −6.00; 0.00 | −6.00; 0.00 | −10.00; −2.33 | −10.00; −3.50 | −12.40; −6.04 | −10.00; −10.00 | 15.00; −3.00 | −17.00; −5.83 |
| Min; Max | −18.0; 7.0 | −16.3; 2.0 | −19.0; 2.0 | −18.0; 2.0 | −15.0; −2.8 | −10.0; −10.0 | −20.0; 0.0 | −21.0; −4.0 |
| Percent change | | | | | | | | |
| from baseline | | | | | | | | |
| Number | 34 (34/0) | 37 (36/1) | 33 (33/0) | 32 (32/0) | 4 (4/0) | 1 (1/0) | 5 (4/1) | 6 (6/0) |
| (observed/ | | | | | | | | |
| imputed) | | | | | | | | |
| Mean (SD) | −18.42 (48.71) | −26.30 (29.41) | −47.44 (37.17) | −50.43 (33.03) | −48.92 (28.25) | −50.00 (NC) | −60.22 (47.52) | −73.06 (22.97) |
| Median | −2.38 | −16.67 | −42.86 | −50.69 | −49.03 | −50.00 | −88.24 | −76.19 |
| Q1; Q3 | −59.17; 0.00 | −45.45; 0.00 | −85.71; −15.38 | −71.43; −33.33 | −67.36; −30.48 | −50.00; −50.00 | −95.24; −17.65 | −93.14; −50.00 |
| Min; Max | −100.0; 100.0 | −100.0; 25.0 | −100.0; 14.3 | −100.0; 16.7 | −83.3; −14.3 | −50.0; −50.0 | −100.0; 0.0 | −100.0; −42.9 |

Week 5

| | | | | | | | | |
|---|---|---|---|---|---|---|---|---|
| Number | 33 (33/0) | 36 (35/1) | 34 (33/1) | 33 (33/0) | 4 (4/0) | 1 (1/0) | 4 (3/1) | 6 (5/1) |
| (observed/ | | | | | | | | |
| imputed) | | | | | | | | |
| Mean (SD) | 8.56 (4.98) | 9.63 (5.13) | 8.34 (6.75) | 6.49 (5.14) | 14.17 (6.62) | 8.00 (NC) | 4.25 (8.50) | 3.42 (4.45) |
| Median | 7.00 | 8.20 | 7.00 | 6.00 | 13.83 | 8.00 | 0.00 | 2.50 |
| Q1; Q3 | 5.83; 12.00 | 6.50; 14.00 | 2.33; 13.00 | 2.80; 8.75 | 8.50; 19.83 | 8.00; 8.00 | 0.00; 8.50 | 0.00; 3.50 |
| Min; Max | 0.0; 21.0 | 0.0; 21.0 | 0.0; 21.0 | 0.0; 19.0 | 8.0; 21.0 | 8.0; 8.0 | 0.0; 17.0 | 0.0; 12.0 |
| Change from | | | | | | | | |
| baseline | | | | | | | | |
| Number | 33 (33/0) | 36 (35/1) | 34 (33/1) | 33 (33/0) | 4 (4/0) | 1 (1/0) | 4 (3/1) | 6 (5/1) |
| (observed/ | | | | | | | | |
| imputed) | | | | | | | | |
| Mean (SD) | −5.32 (4.97) | −5.12 (5.22) | −6.01 (5.82) | −7.57 (5.71) | −5.04 (5.36) | −12.00 (NC) | −12.50 (9.11) | −12.61 (7.49) |
| Median | −4.00 | −3.50 | −7.00 | −7.00 | −5.67 | −12.00 | −14.50 | −12.00 |
| Q1; Q3 | −8.17; −1.00 | −9.33; −0.63 | −9.83; −1.00 | −11.20; −4.00 | −9.50; −0.58 | −12.00; −12.00 | −19.00; −6.00 | −21.00; −5.00 |
| Min; Max | −17.0; 4.0 | −18.0; 1.0 | −17.5; 7.0 | −21.0; 2.0 | −10.0; 1.2 | −12.0; −12.0 | −21.0; 0.0 | −21.0; −4.7 |
| Percent change | | | | | | | | |
| from baseline | | | | | | | | |
| Number | 33 (33/0) | 36 (35/1) | 34 (33/1) | 33 (33/0) | 4 (4/0) | 1 (1/0) | 4 (3/1) | 6 (5/1) |
| (observed/ | | | | | | | | |
| imputed) | | | | | | | | |
| Mean (SD) | −36.12 (33.18) | −32.44 (31.06) | −43.27 (41.28) | −52.28 (34.93) | −27.70 (29.87) | −60.00 (NC) | −75.00 (50.00) | −75.12 (24.22) |
| Median | −41.67 | −26.11 | −42.86 | −50.00 | −30.56 | −60.00 | −100.00 | −75.37 |
| Q1; Q3 | −50.00; −6.25 | −56.92; −4.17 | −83.33; −5.56 | −80.00; −33.33 | −52.78; −2.61 | −60.00; −60.00 | −100.00; | −100.00; |
| | | | | | | | −50.00 | −57.14 |
| Min; Max | −100.0; 50.0 | −100.0; 10.0 | −100.0; 50.0 | −100.0; 18.2 | −55.6; 5.9 | −60.0; −60.0 | −100.0; 0.0 | −100.0; −42.9 |

Week 6

| | | | | | | | | |
|---|---|---|---|---|---|---|---|---|
| Number | 32 (31/1) | 34 (33/1) | 34 (33/1) | 31 (31/0) | 4 (3/1) | 1 (1/0) | 5 (4/1) | 6 (5/1) |
| (observed/ | | | | | | | | |
| imputed) | | | | | | | | |
| Mean (SD) | 9.09 (5.60) | 9.94 (5.76) | 8.17 (6.62) | 6.78 (5.55) | 14.08 (5.76) | 8.00 (NC) | 5.97 (7.25) | 2.83 (4.62) |
| Median | 8.50 | 8.28 | 7.00 | 6.00 | 13.67 | 8.00 | 3.50 | 1.00 |
| Q1; Q3 | 6.00; 14.00 | 7.00; 14.00 | 1.17; 13.00 | 2.00; 10.50 | 9.50; 18.67 | 8.00; 8.00 | 0.00; 9.33 | 0.00; 3.00 |
| Min; Max | 0.0; 21.0 | 0.0; 21.0 | 0.0; 21.0 | 0.0; 21.0 | 8.0; 21.0 | 8.0; 8.0 | 0.0; 17.0 | 0.0; 12.0 |
| Change from | | | | | | | | |
| baseline | | | | | | | | |
| Number | 32 (31/1) | 34 (33/1) | 34 (33/1) | 31 (31/0) | 4 (3/1) | 1 (1/0) | 5 (4/1) | 6 (5/1) |
| (observed/ | | | | | | | | |
| imputed) | | | | | | | | |
| Mean (SD) | −4.82 (6.12) | −5.20 (5.64) | −6.17 (6.01) | −7.22 (6.24) | −5.13 (4.33) | −12.00 (NC) | −8.83 (9.41) | −13.19 (6.97) |
| Median | −3.50 | −4.50 | −6.42 | −7.00 | −5.25 | −12.00 | −12.00 | −13.00 |
| Q1; Q3 | −8.00; 0.00 | −10.00; 0.00 | −11.00; −1.00 | −11.00; −1.50 | −8.50; −1.75 | −12.00; −12.00 | −17.00; 0.00 | −20.00; −7.17 |
| Min; Max | 20.0; 7.0 | −16.3; 4.0 | −17.0; 4.0 | −19.0; 1.2 | −10.0; 0.0 | −12.0; −12.0 | −17.5; 2.3 | −21.0; −5.0 |
| Percent change | | | | | | | | |
| from baseline | | | | | | | | |
| Number | 32 (31/1) | 34 (33/1) | 34 (33/1) | 31 (31/0) | 4 (3/1) | 1 (1/0) | 5 (4/1) | 6 (5/1) |
| (observed/ | | | | | | | | |
| imputed) | | | | | | | | |
| Mean (SD) | −29.90 (43.54) | −33.60 (35.00) | −43.56 (44.25) | −49.49 (37.97) | −28.02 (24.28) | −60.00 (NC) | −50.00 (62.36) | −81.39 (23.52) |
| Median | −25.76 | −37.09 | −47.73 | −46.15 | −28.27 | −60.00 | −83.33 | −91.50 |
| Q1; Q3 | −54.76; 0.00 | −61.11; 0.00 | −85.71; −7.69 | −90.48; −12.50 | −47.22; −8.82 | −60.00; −60.00 | −100.00; 0.00 | −100.00; |
| | | | | | | | | −62.50 |
| Min; Max | −100.0; 87.5 | −100.0; 37.5 | −100.0; 40.0 | −100.0; 8.3 | −55.6; 0.0 | −60.0; −60.0 | −100.0; 33.3 | −100.0; −42.9 |

Week 7

| | | | | | | | | |
|---|---|---|---|---|---|---|---|---|
| Number | 33 (32/1) | 33 (31/2) | 34 (33/1) | 30 (30/0) | 4 (3/1) | 1 (1/0) | 5 (4/1) | 6 (5/1) |
| (observed/ | | | | | | | | |
| imputed) | | | | | | | | |
| Mean (SD) | 9.27 (5.59) | 10.43 (5.27) | 7.34 (6.12) | 6.40 (4.54) | 15.50 (5.88) | 9.00 (NC) | 4.80 (7.46) | 5.11 (4.08) |
| Median | 8.17 | 8.40 | 7.00 | 5.92 | 16.30 | 9.00 | 0.00 | 4.33 |
| Q1; Q3 | 5.60; 14.00 | 7.00; 14.00 | 2.00; 12.60 | 3.00; 9.00 | 10.70; 20.30 | 9.00; 9.00 | 0.00; 7.00 | 3.00; 7.00 |
| Min; Max | 0.0; 21.0 | 0.0; 21.0 | 0.0; 21.0 | 0.0; 15.8 | 8.4; 21.0 | 9.0; 9.0 | 0.0; 17.0 | 0.0; 12.0 |
| Change from | | | | | | | | |
| baseline | | | | | | | | |
| Number | 33 (32/1) | 33 (31/2) | 34 (33/1) | 30 (30/0) | 4 (3/1) | 1 (1/0) | 5 (4/1) | 6 (5/1) |
| (observed/ | | | | | | | | |
| imputed) | | | | | | | | |
| Mean (SD) | −4.60 (5.67) | −4.82 (5.35) | −6.75 (5.85) | −7.60 (5.60) | −3.71 (4.55) | −11.00 (NC) | −10.00 (9.67) | −10.92 (7.20) |
| Median | −5.00 | −4.00 | −7.00 | −6.58 | −2.62 | −11.00 | −12.00 | −9.50 |
| Q1; Q3 | −7.00; 0.00 | −10.00; 0.00 | −9.83; −2.00 | −11.00; −3.00 | −7.30; −0.12 | −11.00; −11.00 | −17.00; 0.00 | −18.00; −4.00 |
| Min; Max | −20.0; 7.4 | −15.0; 4.0 | −20.0; 2.0 | −18.0; 1.8 | −9.6; 0.0 | −11.0;−11.0 | −21.0; 0.0 | −21.0; −3.5 |
| Percent change | | | | | | | | |
| from baseline | | | | | | | | |
| Number | 33 (32/1) | 33 (31/2) | 34 (33/1) | 30 (30/0) | 4 (3/1) | 1 (1/0) | 5 (4/1) | 6 (5/1) |
| (observed/ | | | | | | | | |
| imputed) | | | | | | | | |
| Mean (SD) | −29.77 (42.69) | −29.76 (31.10) | −48.07 (38.31) | −52.36 (32.23) | −20.57 (25.33) | −55.00 (NC) | −60.00 (54.77) | −63.38 (24.01) |
| Median | −25.00 | −35.71 | −50.00 | −50.69 | −14.48 | −55.00 | −100.00 | −54.41 |
| Q1; Q3 | −56.25; 0.00 | −55.56; 0.00 | −84.44; −11.11 | −80.95; −28.57 | −40.56; −0.59 | −55.00; −55.00 | −100.00; 0.00 | −85.71; −42.86 |
| Min; Max | −100.0; 92.5 | −100.0; 23.5 | −100.0; 15.4 | −100.0; 12.5 | −53.3; 0.0 | −55.0; −55.0 | −100.0; 0.0 | −100.0; −42.9 |

Week 8

| | | | | | | | | |
|---|---|---|---|---|---|---|---|---|
| Number | 33 (32/1) | 33 (30/3) | 33 (32/1) | 28 (28/0) | 4 (3/1) | 1 (1/0) | 5 (4/1) | 6 (5/1) |
| (observed/ | | | | | | | | |
| imputed) | | | | | | | | |
| Mean (SD) | 9.16 (6.13) | 10.47 (5.71) | 6.66 (6.70) | 6.12 (4.80) | 11.61 (6.92) | 9.00 (NC) | 4.80 (7.46) | 3.17 (4.45) |
| Median | 8.00 | 9.00 | 5.00 | 6.00 | 9.80 | 9.00 | 0.00 | 1.50 |
| Q1; Q3 | 4.00; 14.00 | 7.00; 14.00 | 2.00; 11.00 | 2.17; 8.58 | 6.42; 16.80 | 9.00; 9.00 | 0.00; 7.00 | 1.00; 3.00 |
| Min; Max | 0.0; 21.0 | 0.0; 21.0 | 0.0; 21.0 | 0.0; 19.0 | 5.8; 21.0 | 9.0; 9.0 | 0.0; 17.0 | 0.0; 12.0 |
| Change from | | | | | | | | |
| baseline | | | | | | | | |
| Number | 33 (32/1) | 33 (30/3) | 33 (32/1) | 28 (28/0) | 4 (3/1) | 1 (1/0) | 5 (4/1) | 6 (5/1) |
| (observed/ | | | | | | | | |
| imputed) | | | | | | | | |
| Mean (SD) | −4.72 (6.09) | −4.56 (5.35) | −7.49 (6.10) | −7.88 (5.18) | −7.60 (5.49) | −11.00 (NC) | −10.00 (9.67) | −12.86 (6.28) |
| Median | −3.00 | −4.00 | −7.00 | −7.50 | −9.12 | −11.00 | −12.00 | −12.50 |
| Q1; Q3 | −7.00; 0.00 | −8.17; 0.00 | −12.00; −2.00 | −10.83; −4.33 | −11.58; −3.62 | −11.00; −11.00 | −17.00; 0.00 | −18.00; −7.17 |
| Min; Max | −19.0; 6.0 | −15.0; 4.0 | −21.0; 2.0 | −17.0; 0.0 | −12.2; 0.0 | −11.0; −11.0 | −21.0; 0.0 | −21.0; −6.0 |
| Percent change | | | | | | | | |
| from baseline | | | | | | | | |
| Number | 33 (32/1) | 33 (30/3) | 33 (32/1) | 28 (28/0) | 4 (3/1) | 1 (1/0) | 5 (4/1) | 6 (5/1) |
| (observed/ | | | | | | | | |
| imputed) | | | | | | | | |
| Mean (SD) | −31.47 (41.65) | −29.79 (35.34) | −54.71 (40.69) | −56.04 (32.39) | −41.29 (30.62) | −55.00 (NC) | −60.00 (54.77) | −80.91 (20.45) |
| Median | −23.81 | −28.57 | −74.07 | −56.35 | −48.79 | −55.00 | −100.00 | −86.73 |
| Q1; Q3 | −56.25; 0.00 | −57.14; 0.00 | −88.89; −14.29 | −82.14; −36.04 | −64.35; −18.24 | −55.00; −55.00 | −100.00; 0.00 | −94.12; −75.00 |
| Min; Max | −100.0; 75.0 | −100.0; 27.3 | −100.0; 16.7 | −100.0; 0.0 | −67.6; 0.0 | −55.0; −55.0 | −100.0; 0.0 | −100.0; −42.9 |

Week 9

| | | | | | | | | |
|---|---|---|---|---|---|---|---|---|
| Number | 32 (31/1) | 34 (31/3) | 29 (28/1) | 29 (29/0) | 4 (3/1) | 1 (1/0) | 5 (4/1) | 6 (5/1) |
| (observed/ | | | | | | | | |
| imputed) | | | | | | | | |
| Mean (SD) | 10.10 (5.69) | 9.82 (5.59) | 7.01 (7.58) | 5.88 (4.98) | 10.85 (6.80) | 8.00 (NC) | 4.80 (7.46) | 3.25 (4.37) |
| Median | 9.00 | 9.20 | 3.50 | 6.00 | 7.70 | 8.00 | 0.00 | 2.00 |
| Q1; Q3 | 7.00; 14.00 | 7.00; 14.00 | 0.00; 11.00 | 1.40; 9.33 | 7.00; 14.70 | 8.00; 8.00 | 0.00; 7.00 | 1.17; 2.33 |
| Min; Max | 0.0; 21.0 | 0.0; 21.0 | 0.0; 21.0 | 0.0; 17.0 | 7.0; 21.0 | 8.0; 8.0 | 0.0; 17.0 | 0.0; 12.0 |
| Change from | | | | | | | | |
| baseline | | | | | | | | |
| Number | 32 (31/1) | 34 (31/3) | 29 (28/1) | 29 (29/0) | 4 (3/1) | 1 (1/0) | 5 (4/1) | 6 (5/1) |
| (observed/ | | | | | | | | |
| imputed) | | | | | | | | |
| Mean (SD) | −3.77 (5.51) | −5.33 (5.35) | −6.95 (6.00) | −8.19 (5.87) | −8.36 (5.73) | −12.00 (NC) | −10.00 (9.67) | −12.78 (6.34) |
| Median | −3.00 | 4.92 | 7.00 | −8.00 | −10.30 | −12.00 | −12.00 | −12.00 |
| Q1; Q3 | −7.00; 0.00 | −8.00; 0.00 | −11.00; −1.17 | −13.00; −4.00 | −11.92; −4.80 | −12.00; −12.00 | −17.00; 0.00 | −18.67; −7.00 |
| Min; Max | −18.0; 5.7 | −19.0; 4.0 | −19.0; 3.0 | −20.0; 3.3 | −12.8; 0.0 | −12.0; −12.0 | −21.0; 0.0 | −21.0; −6.0 |
| Percent change | | | | | | | | |
| from baseline | | | | | | | | |
| Number | 32 (31/1) | 34 (31/3) | 29 (28/1) | 29 (29/0) | 4 (3/1) | 1 (1/0) | 5 (4/1) | 6 (5/1) |
| (observed/ | | | | | | | | |
| imputed) | | | | | | | | |
| Mean (SD) | 24.38 (38.78) | −34.46 (32.68) | −53.79 (43.28) | −55.85 (38.63) | −44.79 (30.23) | −60.00 (NC) | 60.00 (54.77) | −80.12 (19.92) |
| Median | −19.81 | −33.59 | −68.18 | −57.14 | −57.22 | −60.00 | −100.00 | −86.97 |
| Q1; Q3 | −48.08; 0.00 | −50.00; 0.00 | −100.00; −5.56 | −91.25; −28.57 | −62.91; −26.67 | −60.00; −60.00 | −100.00; 0.00 | −88.89; −75.00 |
| Min; Max | −100.0; 62.5 | −100.0; 23.5 | −100.0; 25.0 | −100.0; 55.6 | −64.7; 0.0 | −60.0; −60.0 | −100.0; 0.0 | −100.0; −42.9 |

Week 10

| | | | | | | | | |
|---|---|---|---|---|---|---|---|---|
| Number | 33 (32/1) | 34 (31/3) | 28 (27/1) | 30 (30/0) | 4 (3/1) | 1 (1/0) | 5 (4/1) | 6 (4/2) |
| (observed/ | | | | | | | | |
| imputed) | | | | | | | | |
| Mean (SD) | 9.73 (6.39) | 9.88 (5.93) | 7.05 (7.27) | 5.87 (5.06) | 8.39 (8.95) | 8.00 (NC) | 5.20 (7.19) | 3.00 (4.65) |
| Median | 10.50 | 8.00 | 6.50 | 4.50 | 5.70 | 8.00 | 2.00 | 1.00 |
| Q1; Q3 | 5.25; 14.00 | 5.00; 14.00 | 0.00; 12.00 | 2.00; 8.00 | 2.08; 14.70 | 8.00; 8.00 | 0.00; 7.00 | 0.00; 4.00 |
| Min; Max | 0.0; 21.0 | 0.0; 21.0 | 0.0; 21.0 | 0.0; 19.0 | 1.2; 21.0 | 8.0; 8.0 | 0.0; 17.0 | 0.0; 12.0 |
| Change from | | | | | | | | |
| baseline | | | | | | | | |
| Number | 33 (32/1) | 34 (31/3) | 28 (27/1) | 30 (30/0) | 4 (3/1) | 1 (1/0) | 5 (4/1) | 6 (4/2) |
| (observed/ | | | | | | | | |
| imputed) | | | | | | | | |
| Mean (SD) | −4.14 (6.14) | −5.27 (5.66) | −6.95 (5.65) | −8.20 (5.62) | −10.82 (8.12) | −12.00 (NC) | −9.60 (9.13) | −13.03 (7.21) |
| Median | −3.00 | −4.00 | −6.50 | −9.00 | −12.30 | −12.00 | −12.00 | −13.00 |
| Q1; Q3 | −8.00; 0.00 | −10.00; −1.00 | −12.00; −2.50 | 11.00; −4.00 | −16.83; −4.80 | −12.00; −12.00 | −17.00; 0.00 | −20.00; −7.17 |
| Min; Max | −18.0; 6.0 | −17.0; 4.0 | −16.0; 3.0 | −19.0; 1.0 | −18.7; 0.0 | −12.0; −12.0 | −19.0; 0.0 | −21.0; −4.0 |
| Percent change | | | | | | | | |
| from baseline | | | | | | | | |
| Number | 33 (32/1) | 34 (31/3) | 28 (27/1) | 30 (30/0) | 4 (3/1) | 1 (1/0) | 5 (4/1) | 6 (4/2) |
| (observed/ | | | | | | | | |
| imputed) | | | | | | | | |
| Mean (SD) | −27.51 (45.53) | −33.86 (34.68) | −53.45 (42.56) | −57.32 (34.80) | −57.70 (42.16) | −60.00 (NC) | −58.10 (53.18) | −79.31 (25.96) |
| Median | −18.75 | −28.64 | −47.73 | −63.10 | −68.33 | −60.00 | −90.48 | −91.50 |
| Q1; Q3 | −50.00; 0.00 | −63.64; −5.56 | −100.00; −16.30 | −86.67; −25.00 | −88.73; −26.67 | −60.00; −60.00 | −100.00; 0.00 | −100.00; |
| | | | | | | | | −50.00 |
| Min; Max | −100.0; 62.5 | −100.0; 37.5 | −100.0; 25.0 | −100.0; 16.7 | −94.1; 0.0 | −60.0; −60.0 | −100.0; 0.0 | −100.0; −42.9 |

Week 11

| | | | | | | | | |
|---|---|---|---|---|---|---|---|---|
| Number | 33 (32/1) | 33 (30/3) | 29 (28/1) | 31 (31/0) | 4 (3/1) | 1 (1/0) | 5 (4/1) | 6 (4/2) |
| (observed/ | | | | | | | | |
| imputed) | | | | | | | | |
| Mean (SD) | 9.64 (6.32) | 10.18 (6.18) | 7.13 (7.07) | 6.17 (5.40) | 8.79 (8.61) | 9.00 (NC) | 4.80 (7.46) | 2.67 (4.84) |
| Median | 9.00 | 9.33 | 5.83 | 5.83 | 6.50 | 9.00 | 0.00 | 0.00 |
| Q1; Q3 | 4.67; 14.00 | 7.00; 14.00 | 2.00; 11.00 | 1.00; 9.80 | 3.08; 14.50 | 9.00; 9.00 | 0.00; 7.00 | 0.00; 4.00 |
| Min; Max | 0.0; 21.0 | 0.0; 21.0 | 0.0; 21.0 | 0.0; 19.6 | 1.2; 21.0 | 9.0; 9.0 | 0.0; 17.0 | 0.0; 12.0 |
| Change from | | | | | | | | |
| baseline | | | | | | | | |
| Number | 33 (32/1) | 33 (30/3) | 29 (28/1) | 31 (31/0) | 4 (3/1) | 1 (1/0) | 5 (4/1) | 6 (4/2) |
| (observed/ | | | | | | | | |
| imputed) | | | | | | | | |
| Mean (SD) | −4.23 (6.15) | −5.18 (5.64) | −7.34 (6.14) | −7.90 (6.07) | −10.42 (7.82) | −11.00 (NC) | −10.00 (9.67) | −13.36 (7.26) |
| Median | −4.60 | 3.00 | −7.00 | −8.00 | −11.50 | −11.00 | −12.00 | −13.00 |
| Q1; Q3 | −9.00; 0.00 | −9.67; 0.00 | −12.00; −2.00 | −14.00; −3.00 | −15.83; −5.00 | −11.00; −11.00 | −17.00; 0.00 | −21.00; −8.17 |
| Min; Max | −18.0; 8.0 | −18.0; 4.0 | −21.0; 3.0 | −20.0; 5.6 | −18.7; 0.0 | −11.0;−11.0 | −21.0; 0.0 | −21.0; −4.0 |
| Percent change | | | | | | | | |
| from baseline | | | | | | | | |
| Number | 33 (32/1) | 33 (30/3) | 29 (28/1) | 31 (31/0) | 4 (3/1) | 1 (1/0) | 5 (4/1) | 6 (4/2) |
| (observed/ | | | | | | | | |
| imputed) | | | | | | | | |
| Mean (SD) | −27.83 (49.26) | −33.21 (36.21) | −52.82 (41.27) | −55.30 (37.59) | −55.47 (40.21) | −55.00 (NC) | −60.00 (54.77) | −82.14 (27.76) |
| Median | −33.33 | −30.00 | −54.55 | −61.11 | −63.89 | −55.00 | −100.00 | −100.00 |
| Q1; Q3 | −60.00; 0.00 | −50.88; 0.00 | −89.47; −18.18 | −93.75; −18.33 | −83.17; −27.78 | −55.00; −55.00 | −100.00; 0.00 | −100.00; |
| | | | | | | | | −50.00 |
| Min; Max | −100.0; 96.9 | −100.0; 25.0 | −100.0; 25.0 | −100.0; 40.0 | −94.1; 0.0 | −55.0; −55.0 | −100.0; 0.0 | −100.0; −42.9 |

Week 12

| | | | | | | | | |
|---|---|---|---|---|---|---|---|---|
| Number | 32 (31/1) | 30 (27/3) | 26 (24/2) | 28 (27/1) | 3 (2/1) | 1 (1/0) | 4 (3/1) | 5 (3/2) |
| (observed/ | | | | | | | | |
| imputed) | | | | | | | | |
| Mean (SD) | 9.31 (6.52) | 10.52 (6.14) | 7.52 (7.21) | 5.23 (4.86) | 9.33 (10.69) | 9.00 (NC) | 4.25 (8.50) | 3.20 (5.22) |
| Median | 7.00 | 8.58 | 7.00 | 4.08 | 7.00 | 9.00 | 0.00 | 0.00 |
| Q1; Q3 | 4.33; 14.00 | 7.00; 14.00 | 0.00; 13.00 | 0.58; 8.58 | 0.00; 21.00 | 9.00; 9.00 | 0.00; 8.50 | 0.00; 4.00 |
| Min; Max | 0.0; 21.0 | 0.0; 21.0 | 0.0; 21.0 | 0.0; 15.2 | 0.0; 21.0 | 9.0; 9.0 | 0.0; 17.0 | 0.0; 12.0 |
| Change from | | | | | | | | |
| baseline | | | | | | | | |
| Number | 32 (31/1) | 30 (27/3) | 26 (24/2) | 28 (27/1) | 3 (2/1) | 1 (1/0) | 4 (3/1) | 5 (3/2) |
| (observed/ | | | | | | | | |
| imputed) | | | | | | | | |
| Mean (SD) | −4.60 (6.76) | −4.85 (5.63) | −7.09 (6.54) | −8.77 (5.84) | −10.28 (9.94) | −11.00 (NC) | −12.50 (9.11) | −14.40 (7.60) |
| Median | −2.75 | −4.00 | −4.50 | −8.00 | −11.00 | −11.00 | −14.50 | −17.00 |
| Q1; Q3 | −8.67; 0.00 | −10.25; 0.00 | −11.67; −3.00 | −13.50; −3.72 | −19.83; 0.00 | −11.00; −11.00 | −19.00; −6.00 | −21.00; −9.00 |
| Min; Max | −18.0; 10.7 | −17.0; 4.0 | −21.0; 1.0 | −21.0; 0.0 | −19.8; 0.0 | −11.0;−11.0 | −21.0; 0.0 | −21.0; −4.0 |
| Percent change | | | | | | | | |
| from baseline | | | | | | | | |
| Number | 32 (31/1) | 30 (27/3) | 26 (24/2) | 28 (27/1) | 3 (2/1) | 1 (1/0) | 4 (3/1) | 5 (3/2) |
| (observed/ | | | | | | | | |
| imputed) | | | | | | | | |
| Mean (SD) | −29.00 (51.42) | −31.20 (34.88) | −49.56 (40.81) | −60.93 (34.27) | −53.70 (50.41) | −55.00 (NC) | −75.00 (50.00) | −78.57 (29.45) |
| Median | −21.43 | −33.18 | −37.50 | −64.29 | −61.11 | −55.00 | −100.00 | −100.00 |
| Q1; Q3 | −66.67; 0.00 | −53.95; 0.00 | −100.00; −16.67 | −96.76; −31.67 | −100.00; 0.00 | −55.00; −55.00 | −100.00; | −100.00; |
| | | | | | | | −50.00 | −50.00 |
| Min; Max | −100.0; 133.3 | −100.0; 27.3 | −100.0; 7.7 | −100.0; 0.0 | −100.0; 0.0 | −55.0; −55.0 | −100.0; 0.0 | −100.0; −42.9 |
| End of Study | | | | | | | | |
| Number | 5 (3/2) | 7 (3/4) | 5 (3/2) | 5 (3/2) | 0 (0/0) | 0 (0/0) | 0 (0/0) | 2 (0/2) |
| (observed/ | | | | | | | | |
| imputed) | | | | | | | | |
| Mean (SD) | 9.93 (5.80) | 6.13 (9.09) | 7.80 (8.76) | 10.00 (5.79) | | | | 6.08 (2.95) |
| Median | 11.67 | 1.40 | 7.00 | 10.00 | | | | 6.08 |
| Q1; Q3 | 10.00; 14.00 | 0.00; 17.50 | 0.00; 11.00 | 7.00; 15.00 | | | | 4.00; 8.17 |
| Min; Max | 0.0; 14.0 | 0.0; 21.0 | 0.0; 21.0 | 2.0; 16.0 | | | | 4.0; 8.2 |
| Change from | | | | | | | | |
| baseline | | | | | | | | |
| Number | 5 (3/2) | 7 (3/4) | 5 (3/2) | 5 (3/2) | 0 (0/0) | 0 (0/0) | 0 (0/0) | 2 (0/2) |
| (observed/ | | | | | | | | |
| imputed) | | | | | | | | |
| Mean (SD) | −3.07 (4.88) | −9.87 (8.34) | −7.80 (6.87) | −5.20 (3.11) | | | | −2.00 (2.83) |
| Median | −4.00 | −12.00 | −5.00 | −6.00 | | | | −2.00 |
| Q1; Q3 | −7.00; 0.00 | −18.00; 0.00 | −14.00; −4.00 | −7.00; −5.00 | | | | −4.00; 0.00 |
| Min; Max | −8.0; 3.7 | −19.6; 1.5 | −16.0; 0.0 | −8.0; 0.0 | | | | −4.0; 0.0 |
| Percent change | | | | | | | | |
| from baseline | | | | | | | | |
| Number | 5 (3/2) | 7 (3/4) | 5 (3/2) | 5 (3/2) | 0 (0/0) | 0 (0/0) | 0 (0/0) | 2 (0/2) |
| (observed/ | | | | | | | | |
| imputed) | | | | | | | | |
| Mean (SD) | −23.21 (53.23) | −64.85 (48.75) | −53.67 (44.85) | −38.89 (26.61) | | | | −25.00 (35.36) |
| Median | −28.57 | −93.33 | −41.67 | −44.44 | | | | −25.00 |
| Q1; Q3 | −33.33; 0.00 | −100.00; 0.00 | −100.00; −26.67 | −50.00; −28.57 | | | | −50.00; 0.00 |
| Min; Max | −100.0; 45.8 | 100.0; 9.4 | −100.0; 0.0 | −71.4; 0.0 | | | | −50.0; 0.0 |

Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF.

TABLE 37
Change and Percent Change from Baseline in HSS7 over Time up to Week 12 (ITT Population)

| | | | | | Change difference for | |
|---|---|---|---|---|---|---|
| | | | | Change from | rilzabrutinib vs.placebo | |
| | | Baseline | Postbaseline | baseline | LS Mean | |
| | n | Mean (SD) | Mean (SD) | LS Mean (SE)^{a} | (95% CI)^{a} | pvalueª |
| Week 1 | | | | | | |
| Rilzabrutinib 400 mg TID | 41 | 14.00 (4.77) | 7.76 (4.83) | −6.09 (0.74) | −4.26 (−6.67, −1.85) | <.0001 |
| (N = 41) | | | | | | |
| Rilzabrutinib 400 mg BID | 41 | 14.41 (4.26) | 9.32 (5.77) | −4.79 (0.75) | −2.95 (−5.36, −0.55) | 0.0041 |
| (N = 41) | | | | | | |
| Rilzabrutinib 400 mg QPM | 38 | 14.87 (4.29) | 10.76 (4.83) | −3.98 (0.76) | −2.14 (−4.59, 0.31) | 0.0398 |
| (N = 38) | | | | | | |
| Placebo (N = 40) | 40 | 14.37 (4.89) | 12.46 (5.45) | −1.84 (0.75) | | |
| Week 2 | | | | | | |
| Rilzabrutinib 400 mg TID | 41 | 14.00 (4.77) | 6.61 (5.06) | −7.16 (0.77) | −4.24 (−6.33, −2.15) | <.0001 |
| (N = 41) | | | | | | |
| Rilzabrutinib 400 mg BID | 40 | 14.41 (4.26) | 9.11 (5.66) | −5.15 (0.79) | −2.23 (−4.31, −0.14) | 0.0361 |
| (N = 41) | | | | | | |
| Rilzabrutinib 400 mg QPM | 38 | 14.87 (4.29) | 10.96 (4.91) | −3.87 (0.80) | −0.95 (−3.07, 1.17) | 0.3805 |
| (N = 38) | | | | | | |
| Placebo (N = 40) | 39 | 14.37 (4.89) | 11.27 (5.61) | −2.92 (0.79) | | |
| Week 3 | | | | | | |
| Rilzabrutinib 400 mg TID | 41 | 14.00 (4.77) | 6.49 (4.72) | −7.29 (0.81) | −3.79 (−5.97, −1.61) | 0.0007 |
| (N = 41) | | | | | | |
| Rilzabrutinib 400 mg BID | 41 | 14.41 (4.26) | 8.42 (6.15) | −5.64 (0.82) | −2.14 (−4.31, 0.04) | 0.0544 |
| (N = 41) | | | | | | |
| Rilzabrutinib 400 mg QPM | 38 | 14.87 (4.29) | 10.40 (4.90) | −4.44 (0.83) | −0.94 (−3.15, 1.28) | 0.4081 |
| (N = 38) | | | | | | |
| Placebo (N = 40) | 39 | 14.37 (4.89) | 10.73 (6.04) | −3.50 (0.83) | | |
| Week 4 | | | | | | |
| Rilzabrutinib 400 mg TID | 38 | 14.00 (4.77) | 6.39 (4.92) | −6.98 (0.82) | −3.53 (−5.74, −1.31) | 0.0018 |
| (N = 41) | | | | | | |
| Rilzabrutinib 400 mg BID | 38 | 14.41 (4.26) | 7.50 (6.18) | −6.16 (0.83) | −2.71 (−4.92, −0.50) | 0.0161 |
| (N = 41) | | | | | | |
| Rilzabrutinib 400 mg QPM | 38 | 14.87 (4.29) | 10.52 (4.68) | −4.23 (0.84) | −0.78 (−3.03, 1.46) | 0.4947 |
| (N = 38) | | | | | | |
| Placebo (N = 40) | 38 | 14.37 (4.89) | 10.52 (6.24) | −3.45 (0.84) | | |
| Week 5 | | | | | | |
| Rilzabrutinib 400 mg TID | 39 | 14.00 (4.77) | 6.02 (5.11) | −7.73 (0.84) | −3.09 (−5.36, −0.81) | 0.0078 |
| (N = 41) | | | | | | |
| Rilzabrutinib 400 mg BID | 38 | 14.41 (4.26) | 7.91 (6.94) | −5.93 (0.86) | −1.29 (−3.57, 0.99) | 0.2659 |
| (N = 41) | | | | | | |
| Rilzabrutinib 400 mg QPM | 37 | 14.87 (4.29) | 9.59 (5.07) | −4.97 (0.86) | −0.33 (−2.64, 1.97) | 0.7765 |
| (N = 38) | | | | | | |
| Placebo (N = 40) | 37 | 14.37 (4.89) | 9.17 (5.37) | −4.64 (0.86) | | |
| Week 6 | | | | | | |
| Rilzabrutinib 400 mg TID | 37 | 14.00 (4.77) | 6.14 (5.55) | −7.75 (0.91) | −3.48 (−5.94, −1.02) | 0.0055 |
| (N = 41) | | | | | | |
| Rilzabrutinib 400 mg BID | 39 | 14.41 (4.26) | 7.89 (6.65) | −5.90 (0.92) | −1.64 (−4.07, 0.79) | 0.1859 |
| (N = 41) | | | | | | |
| Rilzabrutinib 400 mg QPM | 35 | 14.87 (4.29) | 9.89 (5.69) | −4.76 (0.93) | −0.50 (−3.00, 2.00) | 0.6952 |
| (N = 38) | | | | | | |
| Placebo (N = 40) | 36 | 14.37 (4.89) | 9.65 (5.75) | −4.26 (0.93) | | |
| Week 7 | | | | | | |
| Rilzabrutinib 400 mg TID | 36 | 14.00 (4.77) | 6.18 (4.44) | −7.66 (0.86) | −3.58 (−5.87, −1.29) | 0.0022 |
| (N = 41) | | | | | | |
| Rilzabrutinib 400 mg BID | 39 | 14.41 (4.26) | 7.01 (6.25) | −6.79 (0.86) | −2.71 (−4.98, −0.44) | 0.0193 |
| (N = 41) | | | | | | |
| Rilzabrutinib 400 mg QPM | 34 | 14.87 (4.29) | 10.38 (5.19) | −4.61 (0.88) | −0.53 (−2.87, 1.81) | 0.6575 |
| (N = 38) | | | | | | |
| Placebo (N = 40) | 37 | 14.37 (4.89) | 9.95 (5.88) | −4.08 (0.86) | | |
| Week 8 | | | | | | |
| Rilzabrutinib 400 mg TID | 34 | 14.00 (4.77) | 5.60 (4.81) | −7.81 (0.92) | −3.06 (−5.54, −0.58) | 0.0156 |
| (N = 41) | | | | | | |
| Rilzabrutinib 400 mg BID | 38 | 14.41 (4.26) | 6.41 (6.73) | −7.49 (0.91) | −2.74 (−5.15, −0.33) | 0.0262 |
| (N = 41) | | | | | | |
| Rilzabrutinib 400 mg QPM | 34 | 14.87 (4.29) | 10.43 (5.63) | −4.64 (0.93) | 0.11 (−2.37, 2.59) | 0.9319 |
| (N = 38) | | | | | | |
| Placebo (N = 40) | 37 | 14.37 (4.89) | 9.43 (6.16) | −4.75 (0.92) | | |
| Week 9 | | | | | | |
| Rilzabrutinib 400 mg TID | 35 | 14.00 (4.77) | 5.43 (4.93) | −7.79 (0.94) | −3.67 (−6.21, −1.13) | 0.0046 |
| (N = 41) | | | | | | |
| Rilzabrutinib 400 mg BID | 34 | 14.41 (4.26) | 6.69 (7.49) | −7.16 (0.96) | −3.04 (−5.57, −0.51) | 0.0185 |
| (N = 41) | | | | | | |
| Rilzabrutinib 400 mg QPM | 35 | 14.87 (4.29) | 9.77 (5.51) | −5.06 (0.95) | −0.94 (−3.47, 1.59) | 0.4656 |
| (N = 38) | | | | | | |
| Placebo (N = 40) | 36 | 14.37 (4.89) | 10.18 (5.72) | −4.12 (0.95) | | |
| Week 10 | | | | | | |
| Rilzabrutinib 400 mg TID | 36 | 14.00 (4.77) | 5.39 (5.05) | −8.15 (0.96) | −3.55 (−6.13, −0.97) | 0.0070 |
| (N = 41) | | | | | | |
| Rilzabrutinib 400 mg BID | 33 | 14.41 (4.26) | 6.77 (7.18) | −7.32 (0.99) | −2.72 (−5.32, −0.11) | 0.0410 |
| (N = 41) | | | | | | |
| Rilzabrutinib 400 mg QPM | 35 | 14.87 (4.29) | 9.82 (5.85) | −5.16 (0.98) | −0.56 (−3.19, 2.07) | 0.6752 |
| (N = 38) | | | | | | |
| Placebo (N = 40) | 37 | 14.37 (4.89) | 9.59 (6.57) | −4.60 (0.98) | | |
| Week 11 | | | | | | |
| Rilzabrutinib 400 mg TID | 37 | 14.00 (4.77) | 5.60 (5.41) | −8.09 (0.96) | −3.55 (−6.14, −0.95) | 0.0074 |
| (N = 41) | | | | | | |
| Rilzabrutinib 400 mg BID | 34 | 14.41 (4.26) | 6.79 (7.06) | −7.01 (0.99) | −2.46 (−5.05, 0.13) | 0.0629 |
| (N = 41) | | | | | | |
| Rilzabrutinib 400 mg QPM | 34 | 14.87 (4.29) | 10.15 (6.09) | −4.97 (0.99) | −0.43 (−3.08, 2.22) | 0.7517 |
| (N = 38) | | | | | | |
| Placebo (N = 40) | 37 | 14.37 (4.89) | 9.55 (6.46) | −4.54 (0.98) | | |
| Week 12 | | | | | | |
| Rilzabrutinib 400 mg TID | 33 | 14.00 (4.77) | 4.92 (4.89) | −8.31 (1.00) | −3.42 (−6.11, −0.74) | 0.0126 |
| (N = 41) | | | | | | |
| Rilzabrutinib 400 mg BID | 30 | 14.41 (4.26) | 7.08 (7.32) | −6.78 (1.02) | −1.89 (−4.58, 0.80) | 0.1683 |
| (N = 41) | | | | | | |
| Rilzabrutinib 400 mg QPM | 31 | 14.87 (4.29) | 10.47 (6.04) | −4.75 (1.03) | 0.14 (−2.62, 2.91) | 0.9186 |
| (N = 38) | | | | | | |
| Placebo (N = 40) | 35 | 14.37 (4.89) | 9.31 (6.74) | −4.89 (1.02) | | |

| | | % Change difference for | |
|---|---|---|---|
| | % Change | rilzabrutinib vs.placebo | |
| | from baseline | LS Mean | |
| | LS Mean (SE)^{a} | (95% CI)ª | pvalue^{a} |
| Week 1 | | | |
| Rilzabrutinib 400 mg TID | −39.34 (5.49) | −30.65 (−45.45, | <.0001 |
| (N = 41) | | −15.85) | |
| Rilzabrutinib 400 mg BID | −33.74 (5.55) | −25.04 (−39.70, | 0.0008 |
| (N = 41) | | −10.39) | |
| Rilzabrutinib 400 mg QPM | −25.22 (5.61) | −16.53 (−31.46, | 0.0301 |
| (N = 38) | | −1.59) | |
| Placebo (N = 40) | −8.69 (5.56) | | |
| Week 2 | | | |
| Rilzabrutinib 400 mg TID | −47.49 (5.64) | −30.93 (−46.17, | <.0001 |
| (N = 41) | | −15.70) | |
| Rilzabrutinib 400 mg BID | −34.94 (5.70) | −18.39 (−33.46, | 0.0168 |
| (N = 41) | | −3.31) | |
| Rilzabrutinib 400 mg QPM | −24.23 (5.75) | −7.67 (−23.03, 7.68) | 0.3273 |
| (N = 38) | | | |
| Placebo (N = 40) | −16.55 (5.72) | | |
| Week 3 | | | |
| Rilzabrutinib 400 mg TID | −48.71 (5.82) | −29.56 (−45.28, | 0.0002 |
| (N = 41) | | −13.85) | |
| Rilzabrutinib 400 mg BID | −38.24 (5.88) | −19.09 (−34.67, | 0.0164 |
| (N = 41) | | −3.50) | |
| Rilzabrutinib 400 mg QPM | −28.70 (5.96) | −9.55 (−25.43, 6.33) | 0.2387 |
| (N = 38) | | | |
| Placebo (N = 40) | −19.15 (5.92) | | |
| Week 4 | | | |
| Rilzabrutinib 400 mg TID | −47.90 (5.95) | −29.20 (−45.35, | 0.0004 |
| (N = 41) | | −13.05) | |
| Rilzabrutinib 400 mg BID | −43.10 (6.02) | −24.40 (−40.33, | 0.0027 |
| (N = 41) | | −8.47) | |
| Rilzabrutinib 400 mg QPM | −26.58 (6.02) | −7.88 (−24.05, 8.29) | 0.3397 |
| (N = 38) | | | |
| Placebo (N = 40) | −18.70 (6.04) | | |
| Week 5 | | | |
| Rilzabrutinib 400 mg TID | −52.29 (5.80) | −23.13 (−38.86, | 0.0039 |
| (N = 41) | | −7.41) | |
| Rilzabrutinib 400 mg BID | −41.40 (5.88) | −12.25 (−27.81, | 0.1232 |
| (N = 41) | | 3.32) | |
| Rilzabrutinib 400 mg QPM | −31.95 (5.91) | −2.80 (−18.63, | 0.7292 |
| (N = 38) | | 13.03) | |
| Placebo (N = 40) | −29.15 (5.91) | | |
| Week 6 | | | |
| Rilzabrutinib 400 mg TID | −52.27 (6.61) | −28.20 (−46.23, | 0.0022 |
| (N = 41) | | −10.17) | |
| Rilzabrutinib 400 mg BID | −39.79 (6.64) | −15.72 (−33.44, | 0.0820 |
| (N = 41) | | 1.99) | |
| Rilzabrutinib 400 mg QPM | −31.19 (6.73) | −7.12 (−25.20, | 0.4401 |
| (N = 38) | | 10.96) | |
| Placebo (N = 40) | −24.07 (6.79) | | |
| Week 7 | | | |
| Rilzabrutinib 400 mg TID | −51.12 (6.01) | −27.16 (−43.35, | 0.0010 |
| (N = 41) | | −10.98) | |
| Rilzabrutinib 400 mg BID | −46.27 (6.01) | −22.31 (−38.25, | 0.0061 |
| (N = 41) | | −6.38) | |
| Rilzabrutinib 400 mg QPM | −29.88 (6.15) | −5.92 (−22.43, | 0.4819 |
| (N = 38) | | 10.58) | |
| Placebo (N = 40) | −23.96 (6.09) | | |
| Week 8 | | | |
| Rilzabrutinib 400 mg TID | −54.34 (6.35) | −25.27 (−42.38, | 0.0038 |
| (N = 41) | | −8.15) | |
| Rilzabrutinib 400 mg BID | −52.52 (6.33) | −23.44 (−40.28, | 0.0063 |
| (N = 41) | | −6.61) | |
| Rilzabrutinib 400 mg QPM | −30.95 (6.43) | −1.88 (−19.05, | 0.8305 |
| (N = 38) | | 15.30) | |
| Placebo (N = 40) | −29.07 (6.37) | | |
| Week 9 | | | |
| Rilzabrutinib 400 mg TID | −53.06 (6.70) | −29.14 (−47.15, | 0.0015 |
| (N = 41) | | −11.12) | |
| Rilzabrutinib 400 mg BID | −51.53 (6.66) | −27.61 (−45.14, | 0.0020 |
| (N = 41) | | −10.07) | |
| Rilzabrutinib 400 mg QPM | −34.23 (6.69) | −10.31 (−28.25, | 0.2602 |
| (N = 38) | | 7.64) | |
| Placebo (N = 40) | −23.93 (6.63) | | |
| Week 10 | | | |
| Rilzabrutinib 400 mg TID | −56.54 (6.68) | −28.54 (−46.43, | 0.0018 |
| (N = 41) | | −10.65) | |
| Rilzabrutinib 400 mg BID | −52.53 (6.82) | −24.54 (−42.40, | 0.0071 |
| (N = 41) | | −6.67) | |
| Rilzabrutinib 400 mg QPM | −34.70 (6.79) | −6.70 (−24.87, | 0.4694 |
| (N = 38) | | 11.46) | |
| Placebo (N = 40) | −28.00 (6.74) | | |
| Week 11 | | | |
| Rilzabrutinib 400 mg TID | −54.96 (6.95) | −28.42 (−47.32, | 0.0032 |
| (N = 41) | | 9.53) | |
| Rilzabrutinib 400 mg BID | −49.14 (7.06) | −22.60 (−41.37, | 0.0183 |
| (N = 41) | | −3.83) | |
| Rilzabrutinib 400 mg QPM | −34.56 (7.07) | −8.01 (−26.93, | 0.4062 |
| (N = 38) | | 10.90) | |
| Placebo (N = 40) | −26.54 (7.04) | | |
| Week 12 | | | |
| Rilzabrutinib 400 mg TID | −57.27 (7.06) | −29.54 (−48.60, | 0.0024 |
| (N = 41) | | −10.47) | |
| Rilzabrutinib 400 mg BID | −46.86 (7.34) | −19.13 (−38.34, | 0.0510 |
| (N = 41) | | 0.09) | |
| Rilzabrutinib 400 mg QPM | −33.48 (7.28) | −5.74 (−25.11, | 0.5609 |
| (N = 38) | | 13.62) | |
| Placebo (N = 40) | −27.73 (7.11) | | |

^{a}Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF; other missing data were imputed by MI. Descriptive statistics at each week include participants with WOCF imputation at the week, and participants whose values were imputed by MI at the week were excluded from the descriptive analysis.

TABLE 38
Change and Percent Change from Baseline in HSS7 over Time up to Week 12 (Omalizumab-Naïve Population)

| | | | | | Change difference for | |
|---|---|---|---|---|---|---|
| | | | | Change from | rilzabrutinib vs.placebo | |
| | | Baseline | Postbaseline | baseline | LS Mean | |
| | n | Mean (SD) | Mean (SD) | LS Mean (SE)^{a} | (95% CI)^{a} | pvalueª |
| Week 1 | | | | | | |
| Rilzabrutinib 400 mg TID | 35 | 13.66 (4.47) | 8.26 (4.91) | −5.32 (0.79) | −3.68 (−6.26, −1.11) | 0.0009 |
| (N = 35) | | | | | | |
| Rilzabrutinib 400 mg BID | 35 | 14.28 (4.21) | 9.26 (5.76) | −4.73 (0.80) | −3.10 (−5.66, −0.53) | 0.0048 |
| (N = 35) | | | | | | |
| Rilzabrutinib 400 mg QPM | 37 | 14.73 (4.26) | 10.73 (4.90) | −3.79 (0.77) | −2.16 (−4.69, 0.37) | 0.0444 |
| (N = 37) | | | | | | |
| Placebo (N = 36) | 39 | 13.83 (4.85) | 12.23 (5.49) | −1.63 (0.79) | | |
| Week 2 | | | | | | |
| Rilzabrutinib 400 mg TID | 35 | 13.66 (4.47) | 7.25 (5.05) | −6.28 (0.80) | −3.60 (−5.75, −1.45) | 0.0010 |
| (N = 35) | | | | | | |
| Rilzabrutinib 400 mg BID | 34 | 14.28 (4.21) | 9.27 (5.49) | −4.95 (0.81) | −2.26 (−4.41, −0.12) | 0.0389 |
| (N = 35) | | | | | | |
| Rilzabrutinib 400 mg QPM | 37 | 14.73 (4.26) | 10.90 (4.97) | −3.72 (0.78) | −1.03 (−3.15, 1.08) | 0.3378 |
| (N = 37) | | | | | | |
| Placebo (N = 36) | 35 | 13.83 (4.85) | 11.11 (5.32) | −2.68 (0.80) | | |
| Week 3 | | | | | | |
| Rilzabrutinib 400 mg TID | 35 | 13.66 (4.47) | 7.03 (4.82) | −6.52 (0.85) | −3.28 (−5.57, −1.00) | 0.0048 |
| (N = 35) | | | | | | |
| Rilzabrutinib 400 mg BID | 35 | 14.28 (4.21) | 8.55 (6.09) | −5.45 (0.86) | −2.21 (−4.49, 0.07) | 0.0571 |
| (N = 35) | | | | | | |
| Rilzabrutinib 400 mg QPM | 37 | 14.73 (4.26) | 10.41 (4.97) | −4.25 (0.83) | −1.02 (−3.27, 1.23) | 0.3745 |
| (N = 37) | | | | | | |
| Placebo (N = 36) | 35 | 13.83 (4.85) | 10.62 (5.91) | −3.24 (0.85) | | |
| Week 4 | | | | | | |
| Rilzabrutinib 400 mg TID | 32 | 13.66 (4.47) | 6.85 (4.96) | −6.26 (0.87) | −3.28 (−5.60, −0.96) | 0.0056 |
| (N = 35) | | | | | | |
| Rilzabrutinib 400 mg BID | 33 | 14.28 (4.21) | 7.60 (6.00) | −5.94 (0.87) | −2.96 (−5.27, −0.65) | 0.0120 |
| (N = 35) | | | | | | |
| Rilzabrutinib 400 mg QPM | 37 | 14.73 (4.26) | 10.53 (4.74) | −4.07 (0.83) | −1.08 (−3.36, 1.19) | 0.3500 |
| (N = 37) | | | | | | |
| Placebo (N = 36) | 34 | 13.83 (4.85) | 10.58 (6.36) | −2.98 (0.86) | | |
| Week 5 | | | | | | |
| Rilzabrutinib 400 mg TID | 33 | 13.66 (4.47) | 6.49 (5.14) | −6.99 (0.88) | −2.17 (−4.52, 0.18) | 0.0708 |
| (N = 35) | | | | | | |
| Rilzabrutinib 400 mg BID | 34 | 14.28 (4.21) | 8.34 (6.75) | −5.50 (0.87) | −0.68 (−3.00, 1.65) | 0.5686 |
| (N = 35) | | | | | | |
| Rilzabrutinib 400 mg QPM | 36 | 14.73 (4.26) | 9.63 (5.13) | −4.75 (0.84) | 0.07 (−2.23, 2.38) | 0.9514 |
| (N = 37) | | | | | | |
| Placebo (N = 36) | 33 | 13.83 (4.85) | 8.56 (4.98) | −4.82 (0.87) | | |
| Week 6 | | | | | | |
| Rilzabrutinib 400 mg TID | 31 | 13.66 (4.47) | 6.78 (5.55) | −6.90 (0.98) | −2.51 (−5.12, 0.09) | 0.0581 |
| (N = 35) | | | | | | |
| Rilzabrutinib 400 mg BID | 34 | 14.28 (4.21) | 8.17 (6.62) | −5.67 (0.96) | −1.28 (−3.84, 1.29) | 0.3295 |
| (N = 35) | | | | | | |
| Rilzabrutinib 400 mg QPM | 34 | 14.73 (4.26) | 9.94 (5.76) | −4.47 (0.93) | −0.08 (−2.63, 2.47) | 0.9525 |
| (N = 37) | | | | | | |
| Placebo (N = 36) | 32 | 13.83 (4.85) | 9.09 (5.60) | −4.39 (0.96) | | |
| Week 7 | | | | | | |
| Rilzabrutinib 400 mg TID | 30 | 13.66 (4.47) | 6.40 (4.54) | −7.27 (0.91) | −2.92 (−5.35, −0.49) | 0.0185 |
| (N = 35) | | | | | | |
| Rilzabrutinib 400 mg BID | 34 | 14.28 (4.21) | 7.34 (6.12) | −6.47 (0.89) | −2.12 (−4.51, 0.27) | 0.0817 |
| (N = 35) | | | | | | |
| Rilzabrutinib 400 mg QPM | 33 | 14.73 (4.26) | 10.43 (5.27) | −4.41 (0.88) | −0.06 (−2.46, 2.33) | 0.9584 |
| (N = 37) | | | | | | |
| Placebo (N = 36) | 33 | 13.83 (4.85) | 9.27 (5.59) | −4.35 (0.90) | | |
| Week 8 | | | | | | |
| Rilzabrutinib 400 mg TID | 28 | 13.66 (4.47) | 6.12 (4.80) | −7.02 (1.00) | −2.47 (−5.11, 0.18) | 0.0678 |
| (N = 35) | | | | | | |
| Rilzabrutinib 400 mg BID | 33 | 14.28 (4.21) | 6.66 (6.70) | −7.26 (0.95) | −2.71 (−5.26, −0.16) | 0.0375 |
| (N = 35) | | | | | | |
| Rilzabrutinib 400 mg QPM | 33 | 14.73 (4.26) | 10.47 (5.71) | −4.41 (0.93) | 0.14 (−2.40, 2.69) | 0.9115 |
| (N = 37) | | | | | | |
| Placebo (N = 36) | 33 | 13.83 (4.85) | 9.16 (6.13) | −4.55 (0.95) | | |
| Week 9 | | | | | | |
| Rilzabrutinib 400 mg TID | 29 | 13.66 (4.47) | 5.88 (4.98) | −7.21 (1.00) | −3.53 (−6.17, −0.89) | 0.0088 |
| (N = 35) | | | | | | |
| Rilzabrutinib 400 mg BID | 29 | 14.28 (4.21) | 7.01 (7.58) | −6.84 (0.99) | −3.16 (−5.79, −0.53) | 0.0185 |
| (N = 35) | | | | | | |
| Rilzabrutinib 400 mg QPM | 34 | 14.73 (4.26) | 9.82 (5.59) | −4.88 (0.96) | −1.19 (−3.81, 1.42) | 0.3703 |
| (N = 37) | | | | | | |
| Placebo (N = 36) | 32 | 13.83 (4.85) | 10.10 (5.69) | −3.68 (0.98) | | |
| Week 10 | | | | | | |
| Rilzabrutinib 400 mg TID | 30 | 13.66 (4.47) | 5.87 (5.06) | −7.54 (1.02) | −3.52 (−6.23, −0.82) | 0.0107 |
| (N = 35) | | | | | | |
| Rilzabrutinib 400 mg BID | 28 | 14.28 (4.21) | 7.05 (7.27) | −7.11 (1.03) | −3.09 (−5.81, −0.38) | 0.0256 |
| (N = 35) | | | | | | |
| Rilzabrutinib 400 mg QPM | 34 | 14.73 (4.26) | 9.88 (5.93) | −4.92 (0.98) | −0.91 (−3.55, 1.74) | 0.5023 |
| (N = 37) | | | | | | |
| Placebo (N = 36) | 33 | 13.83 (4.85) | 9.73 (6.39) | −4.02 (1.00) | | |
| Week 11 | | | | | | |
| Rilzabrutinib 400 mg TID | 31 | 13.66 (4.47) | 6.17 (5.40) | −7.31 (1.02) | −3.31 (−6.05, −0.57) | 0.0179 |
| (N = 35) | | | | | | |
| Rilzabrutinib 400 mg BID | 29 | 14.28 (4.21) | 7.13 (7.07) | −6.77 (1.02) | −2.77 (−5.49, −0.04) | 0.0465 |
| (N = 35) | | | | | | |
| Rilzabrutinib 400 mg QPM | 33 | 14.73 (4.26) | 10.18 (6.18) | −4.81 (1.00) | −0.81 (−3.52, 1.89) | 0.5569 |
| (N = 37) | | | | | | |
| Placebo (N = 36) | 33 | 13.83 (4.85) | 9.64 (6.32) | −4.00 (1.01) | | |
| Week 12 | | | | | | |
| Rilzabrutinib 400 mg TID | 28 | 13.66 (4.47) | 5.23 (4.86) | −7.64 (1.06) | −3.24 (−6.08, −0.39) | 0.0257 |
| (N = 35) | | | | | | |
| Rilzabrutinib 400 mg BID | 26 | 14.28 (4.21) | 7.52 (7.21) | −6.52 (1.07) | −2.12 (−4.97, 0.73) | 0.1450 |
| (N = 35) | | | | | | |
| Rilzabrutinib 400 mg QPM | 30 | 14.73 (4.26) | 10.52 (6.14) | −4.49 (1.04) | −0.09 (−2.92, 2.75) | 0.9531 |
| (N = 37) | | | | | | |
| Placebo (N = 36) | 32 | 13.83 (4.85) | 9.31 (6.52) | −4.40 (1.06) | | |

| | | % Change difference for | |
|---|---|---|---|
| | % Change | rilzabrutinib vs.placebo | |
| | from baseline | LS Mean | |
| | LS Mean (SE)^{a} | (95% CI)^{a} | pvalueª |
| Week 1 | | | |
| Rilzabrutinib 400 mg TID | −34.85 (6.00) | −27.41 (−43.41, | 0.0008 |
| (N = 35) | | −11.41) | |
| Rilzabrutinib 400 mg BID | −34.51 (5.97) | −27.07 (−42.90, | 0.0008 |
| (N = 35) | | −11.24) | |
| Rilzabrutinib 400 mg QPM | −24.49 (5.76) | −17.05 (−32.67, | 0.0325 |
| (N = 37) | | −1.42) | |
| Placebo (N = 36) | −7.44 (5.89) | | |
| Week 2 | | | |
| Rilzabrutinib 400 mg TID | −42.63 (6.08) | −27.36 (−43.65, | 0.0010 |
| (N = 35) | | −11.07) | |
| Rilzabrutinib 400 mg BID | −34.80 (6.07) | −19.53 (−35.64, | 0.0175 |
| (N = 35) | | −3.42) | |
| Rilzabrutinib 400 mg QPM | −23.62 (5.84) | −8.35 (−24.24, 7.54) | 0.3028 |
| (N = 37) | | | |
| Placebo (N = 36) | −15.27 (6.00) | | |
| Week 3 | | | |
| Rilzabrutinib 400 mg TID | −45.07 (6.36) | −27.37 (−44.36, | 0.0016 |
| (N = 35) | | −10.37) | |
| Rilzabrutinib 400 mg BID | −38.38 (6.33) | −20.68 (−37.52, | 0.0161 |
| (N = 35) | | −3.84) | |
| Rilzabrutinib 400 mg QPM | −28.10 (6.12) | −10.40 (−27.02, | 0.2204 |
| (N = 37) | | 6.23) | |
| Placebo (N = 36) | −17.70 (6.28) | | |
| Week 4 | | | |
| Rilzabrutinib 400 mg TID | −44.69 (6.50) | −28.82 (−46.18, | 0.0011 |
| (N = 35) | | −11.45) | |
| Rilzabrutinib 400 mg BID | −43.11 (6.45) | −27.23 (−44.36, | 0.0018 |
| (N = 35) | | −10.10) | |
| Rilzabrutinib 400 mg QPM | −26.07 (6.19) | −10.20 (−27.12, | 0.2378 |
| (N = 37) | | 6.73) | |
| Placebo (N = 36) | −15.88 (6.41) | | |
| Week 5 | | | |
| Rilzabrutinib 400 mg TID | −49.42 (6.23) | −18.86 (−35.66, | 0.0277 |
| (N = 35) | | −2.07) | |
| Rilzabrutinib 400 mg BID | −39.98 (6.23) | −9.42 (−26.13, 7.29) | 0.2692 |
| (N = 35) | | | |
| Rilzabrutinib 400 mg QPM | −31.12 (6.01) | −0.56 (−17.08, | 0.9468 |
| (N = 37) | | 15.96) | |
| Placebo (N = 36) | −30.56 (6.26) | | |
| Week 6 | | | |
| Rilzabrutinib 400 mg TID | −48.15 (7.17) | −24.11 (−43.39, | 0.0142 |
| (N = 35) | | −4.84) | |
| Rilzabrutinib 400 mg BID | −40.02 (7.13) | −15.99 (−35.02, | 0.0996 |
| (N = 35) | | 3.04) | |
| Rilzabrutinib 400 mg QPM | −30.30 (6.90) | −6.27 (−25.06, | 0.5135 |
| (N = 37) | | 12.53) | |
| Placebo (N = 36) | −24.04 (7.13) | | |
| Week 7 | | | |
| Rilzabrutinib 400 mg TID | −50.32 (6.67) | −24.63 (−42.34, | 0.0064 |
| (N = 35) | | −6.91) | |
| Rilzabrutinib 400 mg BID | −45.57 (6.48) | −19.88 (−37.24, | 0.0248 |
| (N = 35) | | −2.52) | |
| Rilzabrutinib 400 mg QPM | −29.51 (6.35) | −3.82 (−21.11, | 0.6649 |
| (N = 37) | | 13.47) | |
| Placebo (N = 36) | −25.69 (6.51) | | |
| Week 8 | | | |
| Rilzabrutinib 400 mg TID | −50.37 (7.07) | −22.36 (−41.09, | 0.0192 |
| (N = 35) | | −3.64) | |
| Rilzabrutinib 400 mg BID | −53.21 (6.86) | −25.21 (−43.54, | 0.0071 |
| (N = 35) | | −6.87) | |
| Rilzabrutinib 400 mg QPM | −30.42 (6.75) | −2.41 (−20.57, | 0.7944 |
| (N = 37) | | 15.74) | |
| Placebo (N = 36) | −28.01 (6.80) | | |
| Week 9 | | | |
| Rilzabrutinib 400 mg TID | −50.43 (7.09) | −27.94 (−47.00, | 0.0041 |
| (N = 35) | | −8.88) | |
| Rilzabrutinib 400 mg BID | −52.03 (7.11) | −29.54 (−48.43, | 0.0022 |
| (N = 35) | | −10.65) | |
| Rilzabrutinib 400 mg QPM | −33.42 (6.85) | −10.93 (−29.60, | 0.2511 |
| (N = 37) | | 7.74) | |
| Placebo (N = 36) | −22.49 (7.08) | | |
| Week 10 | | | |
| Rilzabrutinib 400 mg TID | −53.73 (7.33) | −29.43 (−49.17, | 0.0035 |
| (N = 35) | | −9.70) | |
| Rilzabrutinib 400 mg BID | −52.91 (7.33) | −28.62 (−48.06, | 0.0039 |
| (N = 35) | | −9.18) | |
| Rilzabrutinib 400 mg QPM | −34.15 (7.01) | −9.86 (−29.14, 9.43) | 0.3166 |
| (N = 37) | | | |
| Placebo (N = 36) | −24.29 (7.29) | | |
| Week 11 | | | |
| Rilzabrutinib 400 mg TID | −51.69 (7.63) | −27.64 (−48.19, | 0.0084 |
| (N = 35) | | −7.09) | |
| Rilzabrutinib 400 mg BID | −49.42 (7.58) | −25.37 (−45.60, | 0.0140 |
| (N = 35) | | −5.14) | |
| Rilzabrutinib 400 mg QPM | −33.39 (7.38) | −9.35 (−29.26, | 0.3575 |
| (N = 37) | | 10.56) | |
| Placebo (N = 36) | −24.05 (7.51) | | |
| Week 12 | | | |
| Rilzabrutinib 400 mg TID | −54.23 (7.68) | −29.33 (−49.90, | 0.0052 |
| (N = 35) | | −8.75) | |
| Rilzabrutinib 400 mg BID | −46.07 (7.64) | −21.17 (−41.70, | 0.0432 |
| (N = 35) | | −0.64) | |
| Rilzabrutinib 400 mg QPM | −33.18 (7.52) | −8.28 (−28.75, | 0.4280 |
| (N = 37) | | 12.19) | |
| Placebo (N = 36) | −24.90 (7.64) | | |

^{a}Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF; other missing data were imputed by MI. Descriptive statistics at each week include participants with WOCF imputation at the week, and participants whose values were imputed by MI at the week were excluded from the descriptive analysis.

TABLE 39
Change and Percent Change from Baseline in HSS7 over Time up to Week 12 (Omalizumab-Naïve Population with
Outlier Removed from Analysis)

| | | | | | Change difference for | |
|---|---|---|---|---|---|---|
| | | | | Change from | rilzabrutinib vs.placebo | |
| | | Baseline | Postbaseline | baseline | LS Mean | |
| | n | Mean (SD) | Mean (SD) | LS Mean (SE)ª | (95% CI)^{a} | pvalueª |
| Week 1 | | | | | | |
| Rilzabrutinib 400 mg TID | 34 | 14.06 (3.85) | 8.50 (4.76) | −5.38 (0.80) | −3.72 (−6.32, −1.12) | 0.0009 |
| (N = 34) | | | | | | |
| Rilzabrutinib 400 mg BID | 35 | 14.28 (4.21) | 9.26 (5.76) | −4.76 (0.80) | −3.10 (−5.67, −0.52) | 0.0049 |
| (N = 35) | | | | | | |
| Rilzabrutinib 400 mg QPM | 37 | 14.73 (4.26) | 10.73 (4.90) | −3.83 (0.77) | −2.17 (−4.71, 0.37) | 0.0444 |
| (N = 37) | | | | | | |
| Placebo (N = 36) | 36 | 13.83 (4.85) | 12.23 (5.49) | −1.66 (0.79) | | |
| Week 2 | | | | | | |
| Rilzabrutinib 400 mg TID | 34 | 14.06 (3.85) | 7.44 (5.01) | −6.39 (0.81) | −3.68 (−5.85, −1.50) | 0.0009 |
| (N = 34) | | | | | | |
| Rilzabrutinib 400 mg BID | 34 | 14.28 (4.21) | 9.27 (5.49) | −4.98 (0.81) | −2.27 (−4.42, −0.12) | 0.0389 |
| (N = 35) | | | | | | |
| Rilzabrutinib 400 mg QPM | 37 | 14.73 (4.26) | 10.90 (4.97) | −3.77 (0.78) | −1.05 (−3.18, 1.07) | 0.3302 |
| (N = 37) | | | | | | |
| Placebo (N = 36) | 35 | 13.83 (4.85) | 11.11 (5.32) | −2.71 (0.80) | | |
| Week 3 | | | | | | |
| Rilzabrutinib 400 mg TID | 34 | 14.06 (3.85) | 7.03 (4.89) | −6.77 (0.85) | −3.54 (−5.82, −1.26) | 0.0023 |
| (N = 34) | | | | | | |
| Rilzabrutinib 400 mg BID | 35 | 14.28 (4.21) | 8.55 (6.09) | −5.46 (0.85) | −2.23 (−4.49, 0.03) | 0.0529 |
| (N = 35) | | | | | | |
| Rilzabrutinib 400 mg QPM | 37 | 14.73 (4.26) | 10.41 (4.97) | −4.32 (0.82) | −1.09 (−3.32, 1.14) | 0.3394 |
| (N = 37) | | | | | | |
| Placebo (N = 36) | 35 | 13.83 (4.85) | 10.62 (5.91) | −3.23 (0.84) | | |
| Week 4 | | | | | | |
| Rilzabrutinib 400 mg TID | 32 | 14.06 (3.85) | 6.85 (4.96) | −6.48 (0.87) | −3.47 (−5.78, −1.16) | 0.0033 |
| (N = 34) | | | | | | |
| Rilzabrutinib 400 mg BID | 33 | 14.28 (4.21) | 7.60 (6.00) | −5.94 (0.86) | −2.93 (−5.22, −0.65) | 0.0119 |
| (N = 35) | | | | | | |
| Rilzabrutinib 400 mg QPM | 37 | 14.73 (4.26) | 10.53 (4.74) | 4.12 (0.83) | −1.10 (−3.35, 1.14) | 0.3359 |
| (N = 37) | | | | | | |
| Placebo (N = 36) | 34 | 13.83 (4.85) | 10.58 (6.36) | −3.01 (0.85) | | |
| Week 5 | | | | | | |
| Rilzabrutinib 400 mg TID | 33 | 14.06 (3.85) | 6.49 (5.14) | −7.19 (0.88) | −2.34 (−4.69, 0.01) | 0.0514 |
| (N = 34) | | | | | | |
| Rilzabrutinib 400 mg BID | 34 | 14.28 (4.21) | 8.34 (6.75) | −5.51 (0.87) | −0.66 (−2.98, 1.66) | 0.5779 |
| (N = 35) | | | | | | |
| Rilzabrutinib 400 mg QPM | 36 | 14.73 (4.26) | 9.63 (5.13) | −4.83 (0.84) | 0.02 (−2.28, 2.32) | 0.9862 |
| (N = 37) | | | | | | |
| Placebo (N = 36) | 33 | 13.83 (4.85) | 8.56 (4.98) | −4.85 (0.87) | | |
| Week 6 | | | | | | |
| Rilzabrutinib 400 mg TID | 31 | 14.06 (3.85) | 6.78 (5.55) | −7.10 (0.97) | −2.66 (−5.24, −0.09) | 0.0427 |
| (N = 34) | | | | | | |
| Rilzabrutinib 400 mg BID | 34 | 14.28 (4.21) | 8.17 (6.62) | −5.66 (0.96) | −1.22 (−3.78, 1.33) | 0.3482 |
| (N = 35) | | | | | | |
| Rilzabrutinib 400 mg QPM | 34 | 14.73 (4.26) | 9.94 (5.76) | −4.54 (0.93) | −0.10 (−2.65, 2.44) | 0.9363 |
| (N = 37) | | | | | | |
| Placebo (N = 36) | 32 | 13.83 (4.85) | 9.09 (5.60) | −4.44 (0.96) | | |
| Week 7 | | | | | | |
| Rilzabrutinib 400 mg TID | 30 | 14.06 (3.85) | 6.40 (4.54) | −7.42 (0.90) | −3.01 (−5.42, −0.60) | 0.0144 |
| (N = 34) | | | | | | |
| Rilzabrutinib 400 mg BID | 34 | 14.28 (4.21) | 7.34 (6.12) | −6.51 (0.89) | −2.10 (−4.48, 0.27) | 0.0824 |
| (N = 35) | | | | | | |
| Rilzabrutinib 400 mg QPM | 33 | 14.73 (4.26) | 10.43 (5.27) | −4.45 (0.87) | −0.04 (−2.40, 2.32) | 0.9755 |
| (N = 37) | | | | | | |
| Placebo (N = 36) | 33 | 13.83 (4.85) | 9.27 (5.59) | −4.41 (0.89) | | |
| Week 8 | | | | | | |
| Rilzabrutinib 400 mg TID | 28 | 14.06 (3.85) | 6.12 (4.80) | −7.27 (0.98) | −2.65 (−5.28, −0.02) | 0.0486 |
| (N = 34) | | | | | | |
| Rilzabrutinib 400 mg BID | 33 | 14.28 (4.21) | 6.66 (6.70) | −7.25 (0.95) | −2.63 (−5.19, −0.07) | 0.0443 |
| (N = 35) | | | | | | |
| Rilzabrutinib 400 mg QPM | 33 | 14.73 (4.26) | 10.47 (5.71) | −4.49 (0.93) | 0.13 (−2.41, 2.67) | 0.9189 |
| (N = 37) | | | | | | |
| Placebo (N = 36) | 33 | 13.83 (4.85) | 9.16 (6.13) | −4.62 (0.96) | | |
| Week 9 | | | | | | |
| Rilzabrutinib 400 mg TID | 29 | 14.06 (3.85) | 5.88 (4.98) | −7.35 (1.02) | −3.55 (−6.27, −0.83) | 0.0104 |
| (N = 34) | | | | | | |
| Rilzabrutinib 400 mg BID | 29 | 14.28 (4.21) | 7.01 (7.58) | −6.87 (1.00) | −3.07 (−5.75, −0.40) | 0.0241 |
| (N = 35) | | | | | | |
| Rilzabrutinib 400 mg QPM | 34 | 14.73 (4.26) | 9.82 (5.59) | −4.89 (0.96) | −1.09 (−3.72, 1.53) | 0.4145 |
| (N = 37) | | | | | | |
| Placebo (N = 36) | 32 | 13.83 (4.85) | 10.10 (5.69) | −3.80 (0.99) | | |
| Week 10 | | | | | | |
| Rilzabrutinib 400 mg TID | 30 | 14.06 (3.85) | 5.87 (5.06) | −7.62 (1.03) | −3.59 (−6.32, −0.86) | 0.0099 |
| (N = 34) | | | | | | |
| Rilzabrutinib 400 mg BID | 28 | 14.28 (4.21) | 7.05 (7.27) | −7.21 (1.04) | −3.18 (−5.92, −0.45) | 0.0225 |
| (N = 35) | | | | | | |
| Rilzabrutinib 400 mg QPM | 34 | 14.73 (4.26) | 9.88 (5.93) | −5.01 (0.97) | −0.99 (−3.65, 1.68) | 0.4693 |
| (N = 37) | | | | | | |
| Placebo (N = 36) | 33 | 13.83 (4.85) | 9.73 (6.39) | −4.03 (1.01) | | |
| Week 11 | | | | | | |
| Rilzabrutinib 400 mg TID | 31 | 14.06 (3.85) | 6.17 (5.40) | −7.49 (1.03) | −3.43 (−6.19, −0.66) | 0.0151 |
| (N = 34) | | | | | | |
| Rilzabrutinib 400 mg BID | 29 | 14.28 (4.21) | 7.13 (7.07) | −6.73 (1.04) | −2.67 (−5.45, 0.11) | 0.0601 |
| (N = 35) | | | | | | |
| Rilzabrutinib 400 mg QPM | 33 | 14.73 (4.26) | 10.18 (6.18) | −4.85 (0.99) | −0.79 (−3.51, 1.93) | 0.5709 |
| (N = 37) | | | | | | |
| Placebo (N = 36) | 33 | 13.83 (4.85) | 9.64 (6.32) | −4.06 (1.02) | | |
| Week 12 | | | | | | |
| Rilzabrutinib 400 mg TID | 28 | 14.06 (3.85) | 5.23 (4.86) | −7.89 (1.07) | −3.54 (−6.40, −0.67) | 0.0156 |
| (N = 34) | | | | | | |
| Rilzabrutinib 400 mg BID | 26 | 14.28 (4.21) | 7.52 (7.21) | −6.56 (1.07) | −2.20 (−5.04, 0.63) | 0.1279 |
| (N = 35) | | | | | | |
| Rilzabrutinib 400 mg QPM | 30 | 14.73 (4.26) | 10.52 (6.14) | −4.57 (1.02) | −0.22 (−3.01, 2.57) | 0.8768 |
| (N = 37) | | | | | | |
| Placebo (N = 36) | 32 | 13.83 (4.85) | 9.31 (6.52) | −4.35 (1.04) | | |

| | | % Change difference for | |
|---|---|---|---|
| | % Change | rilzabrutinib vs.placebo | |
| | from baseline | LS Mean | |
| | LS Mean (SE)ª | (95% CI)ª | pvalue^{a} |
| Week 1 | | | |
| Rilzabrutinib 400 mg TID | −35.11 (5.99) | −27.60 (−46.98, | 0.0009 |
| (N = 34) | | −8.21) | |
| Rilzabrutinib 400 mg BID | −34.59 (5.97) | −27.08 (−46.27, | 0.0010 |
| (N = 35) | | −7.88) | |
| Rilzabrutinib 400 mg QPM | −24.61 (5.76) | −17.09 (−36.03, | 0.0338 |
| (N = 37) | | 1.85) | |
| Placebo (N = 36) | −7.52 (5.90) | | |
| Week 2 | | | |
| Rilzabrutinib 400 mg TID | −42.74 (6.08) | −27.42 (−43.65, | 0.0009 |
| (N = 34) | | −11.18) | |
| Rilzabrutinib 400 mg BID | −35.26 (6.08) | −19.94 (−36.04, | 0.0152 |
| (N = 35) | | −3.84) | |
| Rilzabrutinib 400 mg QPM | −23.80 (5.84) | −8.48 (−24.35, 7.39) | 0.2948 |
| (N = 37) | | | |
| Placebo (N = 36) | −15.32 (5.99) | | |
| Week 3 | | | |
| Rilzabrutinib 400 mg TID | −45.23 (6.36) | −27.27 (−44.29, | 0.0017 |
| (N = 34) | | −10.26) | |
| Rilzabrutinib 400 mg BID | −38.51 (6.34) | −20.55 (−37.40, | 0.0169 |
| (N = 35) | | −3.70) | |
| Rilzabrutinib 400 mg QPM | −28.21 (6.11) | −10.26 (−26.91, | 0.2272 |
| (N = 37) | | 6.39) | |
| Placebo (N = 36) | −17.96 (6.30) | | |
| Week 4 | | | |
| Rilzabrutinib 400 mg TID | −44.85 (6.50) | −28.68 (−46.02, | 0.0012 |
| (N = 34) | | −11.35) | |
| Rilzabrutinib 400 mg BID | −43.08 (6.46) | −26.91 (−44.03, | 0.0021 |
| (N = 35) | | −9.79) | |
| Rilzabrutinib 400 mg QPM | −26.10 (6.19) | −9.93 (−26.78, 6.91) | 0.2477 |
| (N = 37) | | | |
| Placebo (N = 36) | −16.17 (6.37) | | |
| Week 5 | | | |
| Rilzabrutinib 400 mg TID | −49.43 (6.29) | −19.00 (−35.86, | 0.0272 |
| (N = 34) | | −2.14) | |
| Rilzabrutinib 400 mg BID | −40.02 (6.24) | −9.59 (−26.21, 7.02) | 0.2577 |
| (N = 35) | | | |
| Rilzabrutinib 400 mg QPM | −31.37 (6.02) | 0.94 (−17.38, | 0.9109 |
| (N = 37) | | 15.50) | |
| Placebo (N = 36) | −30.43 (6.23) | | |
| Week 6 | | | |
| Rilzabrutinib 400 mg TID | −48.07 (7.15) | −23.42 (−42.47, | 0.0159 |
| (N = 34) | | −4.38) | |
| Rilzabrutinib 400 mg BID | −40.11 (7.09) | −15.46 (−34.37, | 0.1089 |
| (N = 35) | | 3.44) | |
| Rilzabrutinib 400 mg QPM | −30.40 (6.87) | −5.75 (−24.56, | 0.5487 |
| (N = 37) | | 13.05) | |
| Placebo (N = 36) | −24.65 (7.12) | | |
| Week 7 | | | |
| Rilzabrutinib 400 mg TID | −50.21 (6.54) | −24.42 (−41.91, | 0.0062 |
| (N = 34) | | −6.94) | |
| Rilzabrutinib 400 mg BID | −45.82 (6.45) | −20.03 (−37.24, | 0.0226 |
| (N = 35) | | −2.81) | |
| Rilzabrutinib 400 mg QPM | −29.53 (6.30) | −3.74 (−20.88, | 0.6688 |
| (N = 37) | | 13.40) | |
| Placebo (N = 36) | −25.79 (6.44) | | |
| Week 8 | | | |
| Rilzabrutinib 400 mg TID | −50.94 (7.02) | −22.59 (−41.56, | 0.0196 |
| (N = 34) | | −3.62) | |
| Rilzabrutinib 400 mg BID | −52.80 (6.83) | −24.45 (−42.85, | 0.0092 |
| (N = 35) | | −6.05) | |
| Rilzabrutinib 400 mg QPM | −30.95 (6.65) | −2.60 (−20.89, | 0.7806 |
| (N = 37) | | 15.69) | |
| Placebo (N = 36) | −28.35 (6.91) | | |
| Week 9 | | | |
| Rilzabrutinib 400 mg TID | −50.03 (7.38) | −27.33 (−46.80, | 0.0060 |
| (N = 34) | | −7.85) | |
| Rilzabrutinib 400 mg BID | −51.66 (7.19) | −28.96 (−48.16, | 0.0031 |
| (N = 35) | | −9.75) | |
| Rilzabrutinib 400 mg QPM | −33.40 (6.87) | −10.69 (−29.52, | 0.2658 |
| (N = 37) | | 8.14) | |
| Placebo (N = 36) | −22.70 (7.10) | | |
| Week 10 | | | |
| Rilzabrutinib 400 mg TID | −53.16 (7.35) | −28.45 (−48.02, | 0.0044 |
| (N = 34) | | −8.89) | |
| Rilzabrutinib 400 mg BID | −53.63 (7.40) | −28.93 (−48.47, | 0.0037 |
| (N = 35) | | −9.38) | |
| Rilzabrutinib 400 mg QPM | −34.33 (6.97) | −9.62 (−28.72, 9.48) | 0.3234 |
| (N = 37) | | | |
| Placebo (N = 36) | −24.71 (7.20) | | |
| Week 11 | | | |
| Rilzabrutinib 400 mg TID | −51.42 (7.56) | −27.32 (−47.67, | 0.0085 |
| (N = 34) | | −6.96) | |
| Rilzabrutinib 400 mg BID | −49.25 (7.67) | −25.15 (−45.61, | 0.0160 |
| (N = 35) | | −4.68) | |
| Rilzabrutinib 400 mg QPM | −33.92 (7.28) | −9.82 (−29.88, | 0.3371 |
| (N = 37) | | 10.24) | |
| Placebo (N = 36) | −24.10 (7.53) | | |
| Week 12 | | | |
| Rilzabrutinib 400 mg TID | −54.34 (7.78) | −28.97 (−49.68, | 0.0061 |
| (N = 34) | | −8.27) | |
| Rilzabrutinib 400 mg BID | −47.15 (7.75) | −21.78 (−42.31, | 0.0376 |
| (N = 35) | | −1.25) | |
| Rilzabrutinib 400 mg QPM | −32.82 (7.36) | −7.45 (−27.56, | 0.4674 |
| (N = 37) | | 12.65) | |
| Placebo (N = 36) | −25.37 (7.52) | | |

^{a}Each of the imputed complete data were analyzed by fitting an ANCOVA model with the corresponding baseline value, intervention group and regions as covariates.
Note:
Data collected after study intervention discontinuation were included. Data post selected prohibited/rescue medications (high/medium impact on efficacy confirmed through blinded medical review) were set to missing and imputed by WOCF. Missing data after study intervention discontinuation for lack of efficacy were imputed by WOCF; other missing data were imputed by MI. Descriptive statistics at each week include participants with WOCF imputation at the week, and participants whose values were imputed by MI at the week were excluded from the descriptive analysis.

### AAS7

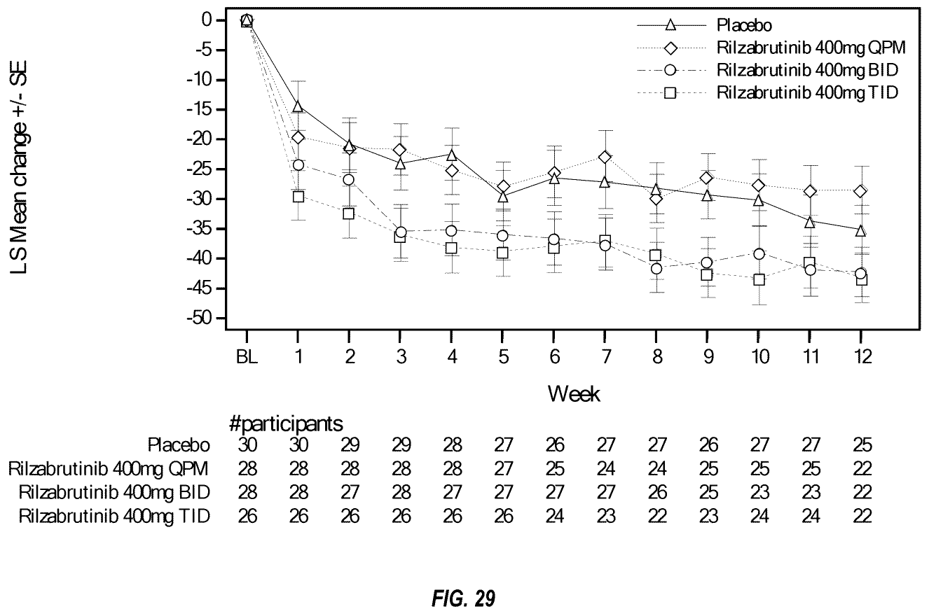

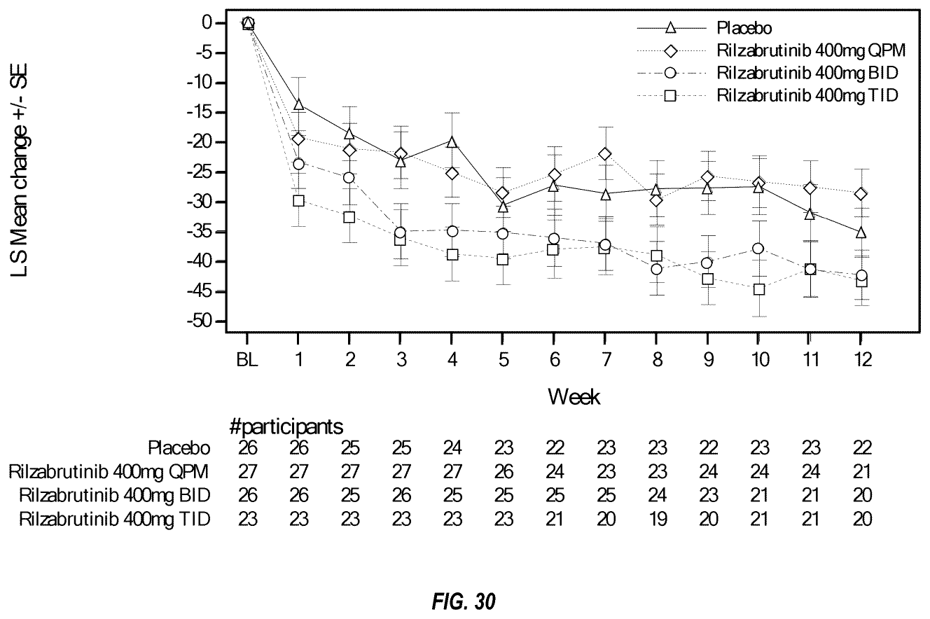

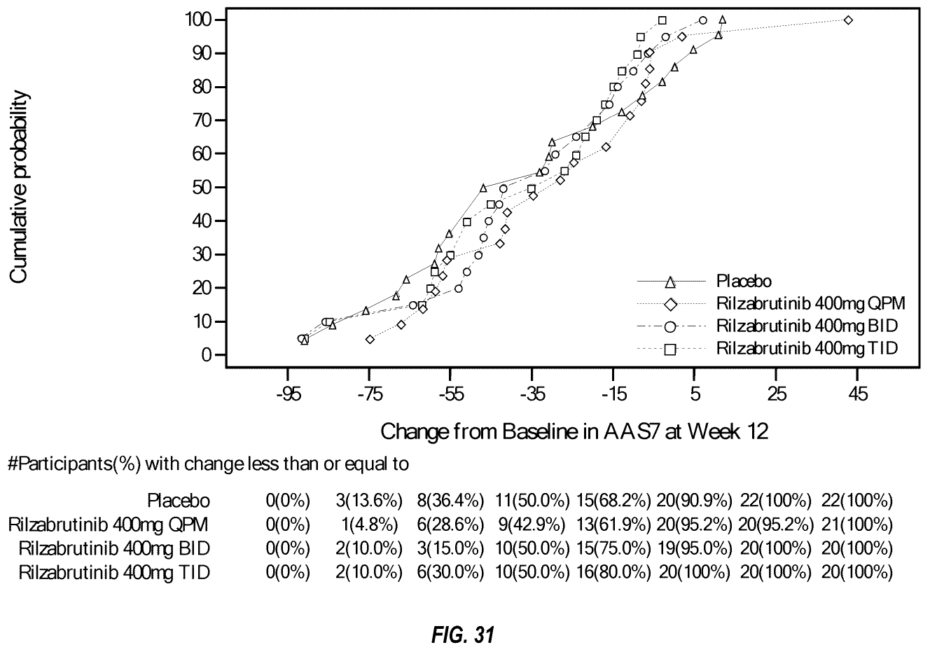

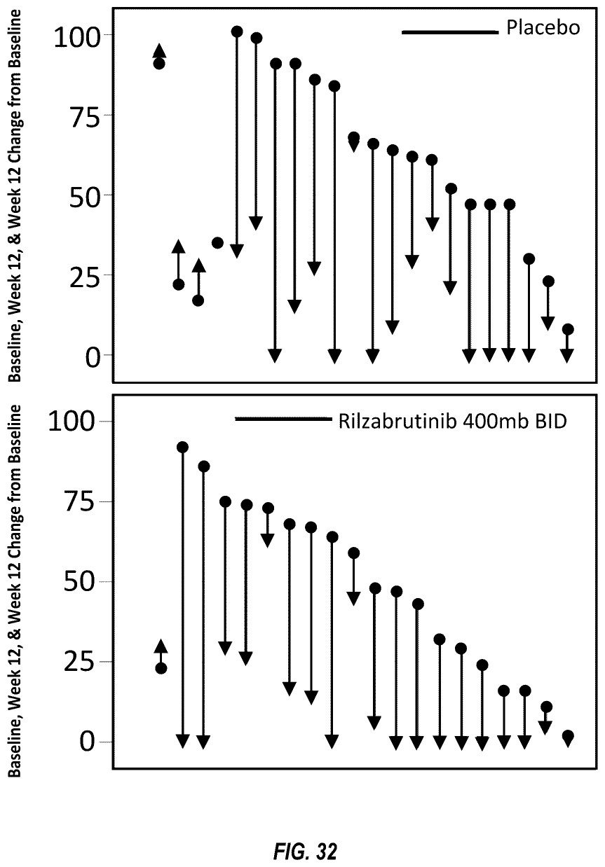

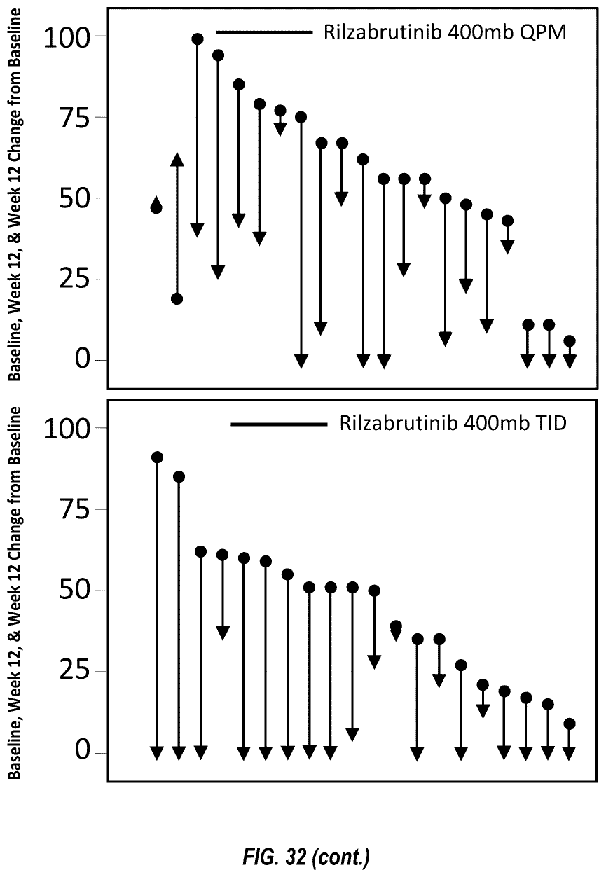

Treatment with rilzabrutinib was associated with meaningful improvements in AAS7 in patients with angioedema at baseline. Notably, participants receiving 400 mg TID rilzabrutinib exhibited a substantial reduction in AAS7 at Week 12 (see FIGS. 29-32 and Tables 40-42). In the ITT population, participants receiving 400 mg TID rilzabrutinib exhibited a LS mean change from baseline at Week 12 of −43.36 (p=0.1511) points. By contrast, patients receiving the placebo had a LS mean change form baseline of −35.31 points (see Table 41). Similar results were observed for the omalizumab-naïve population, in which patients receiving the 400 mg TID rilzabrutinib intervention exhibited an LS mean change from baseline at Week 12 of −44.04 (p=0.0797), which represents an 84.63% decrease from baseline (see Table 42).

Improvement in AAS7 was dose-dependent, with participants randomized into the 400 mg BID rilzabrutinib treatment group also exhibiting a meaningful response.

Additionally, the reduction in AAS7 was rapid, with results observed as early as Week 1, and sustained throughout the study.

TABLE 40
Summary of AAS7 by Week in the Double−Blind Period in Participants with Angioedema at Baseline
(Omalizumab-Naïve Population and Patients with Prior Omalizumab Use, Including Omalizumab-Incomplete Responders)

| | Omalizumab-Naïve Population | | | |
|---|---|---|---|---|
| | | Rilzabrutinib | Rilzabrutinib | Rilzabrutinib |
| | Placebo | 400 mg QPM | 400 mg BID | 400 mg TID |
| AAS7 | (N = 36) | (N = 37) | (N = 35) | (N = 35) |
| Baseline | | | | |
| Number | 26 | 27 | 26 | 23 |
| Mean (SD) | 58.73 (26.52) | 53.48 (25.55) | 46.58 (26.66) | 47.91 (22.78) |
| Median | 61.50 | 56.00 | 46.50 | 51.00 |
| Q1; Q3 | 42.00; 84.00 | 35.00; 74.00 | 24.00; 68.00 | 27.00; 62.00 |
| Min; Max | 8.0; 101.0 | 6.0; 99.0 | 2.0; 92.0 | 9.0; 91.0 |
| Week 1 | | | | |
| Number (observed/imputed) | 26 (26/0) | 27 (27/0) | 26 (26/0) | 23 (23/0) |
| Mean (SD) | 43.46 (28.75) | 34.85 (21.86) | 25.15 (31.33) | 18.96 (23.24) |
| Median | 36.00 | 39.00 | 10.50 | 9.00 |
| Q1; Q3 | 20.00; 65.00 | 17.00; 50.00 | 0.00; 48.00 | 0.00; 35.00 |
| Min; Max | 0.0; 97.0 | 0.0; 77.0 | 0.0; 94.5 | 0.0; 82.0 |
| Change from baseline | | | | |
| Number (observed/imputed) | 26 (26/0) | 27 (27/0) | 26 (26/0) | 23 (23/0) |
| Mean (SD) | −15.27 (23.42) | −18.63 (22.01) | −21.44 (18.46) | −28.96 (27.34) |
| Median | −13.00 | −19.00 | −22.00 | −26.00 |
| Q1; Q3 | −30.00; −2.00 | −39.00; 3.00 | −32.00; −11.00 | −55.00; −9.00 |
| Min; Max | −60.0; 44.0 | −62.0; 12.0 | −61.0; 7.0 | −76.0; 19.0 |
| Percent change from baseline | | | | |
| Number (observed/imputed) | 26 (26/0) | 27 (27/0) | 26 (26/0) | 23 (23/0) |
| Mean (SD) | −18.83 (60.46) | −30.81 (44.93) | 62.04 (42.40) | −56.39 (51.49) |
| Median | −18.98 | −35.35 | −82.71 | −89.41 |
| Q1; Q3 | −59.38; −3.30 | −69.64; 4.48 | −100.00; −26.74 | −100.00; −28.57 |
| Min; Max | −100.0; 200.0 | −100.0; 45.5 | −100.0; 12.5 | −100.0; 84.2 |
| Week 2 | | | | |
| Number (observed/imputed) | 25 (25/0) | 27 (27/0) | 25 (25/0) | 23 (23/0) |
| Mean (SD) | 38.14 (29.33) | 33.25 (23.40) | 24.57 (29.12) | 17.03 (20.90) |
| Median | 38.00 | 31.50 | 14.00 | 9.33 |
| Q1; Q3 | 14.00; 63.00 | 14.00; 49.00 | 0.00; 37.80 | 0.00; 30.80 |
| Min; Max | 0.0; 91.0 | 0.0; 77.0 | 0.0; 91.0 | 0.0; 68.0 |
| Change from baseline | | | | |
| Number (observed/imputed) | 25 (25/0) | 27 (27/0) | 25 (25/0) | 23 (23/0) |
| Mean (SD) | −19.82 (22.48) | −20.23 (24.12) | −22.60 (18.31) | −30.89 (28.32) |
| Median | −17.00 | −11.00 | −23.00 | −27.00 |
| Q1; Q3 | −40.00; −5.00 | −32.67; −1.67 | −33.00; −11.00 | 55.00; −9.00 |
| Min; Max | −66.0; 22.0 | −75.0; 15.0 | −57.0; 11.0 | −75.0; 16.0 |
| Percent change from baseline | | | | |
| Number (observed/imputed) | 25 (25/0) | 27 (27/0) | 25 (25/0) | 23 (23/0) |
| Mean (SD) | −38.51 (43.99) | −36.22 (43.96) | −45.07 (90.72) | −58.84 (53.09) |
| Median | −25.58 | −31.31 | −77.03 | −81.97 |
| Q1; Q3 | −77.42; −9.85 | −64.56; −2.49 | −100.00; −26.56 | −100.00; −28.57 |
| Min; Max | −100.0; 50.0 | −100.0; 88.2 | −100.0; 350.0 | −100.0; 84.2 |
| Week 3 | | | | |
| Number (observed/imputed) | 25 (25/0) | 27 (27/0) | 26 (26/0) | 23 (23/0) |
| Mean (SD) | 33.14 (30.73) | 32.60 (20.89) | 15.95 (20.90) | 14.08 (22.48) |
| Median | 22.40 | 34.00 | 6.50 | 0.00 |
| Q1; Q3 | 5.00; 56.00 | 22.00; 46.00 | 0.00; 27.00 | 0.00; 24.00 |
| Min; Max | 0.0; 93.0 | 0.0; 72.0 | 0.0; 68.0 | 0.0; 67.0 |
| Change from baseline | | | | |
| Number (observed/imputed) | 25 (25/0) | 27 (27/0) | 26 (26/0) | 23 (23/0) |
| Mean (SD) | −24.82 (27.21) | −20.88 (26.24) | −30.63 (24.46) | −33.83 (29.99) |
| Median | −17.00 | −13.00 | −25.88 | −27.00 |
| Q1; Q3 | −47.00; −8.00 | −49.00; −5.00 | −51.00; −16.00 | −59.00; −11.00 |
| Min; Max | −83.0; 21.0 | −75.0; 23.0 | −92.0; 10.8 | −83.0; 26.8 |
| Percent change from baseline | | | | |
| Number (observed/imputed) | 25 (25/0) | 27 (27/0) | 26 (26/0) | 23 (23/0) |
| Mean (SD) | −45.13 (46.73) | −29.46 (63.83) | −45.93 (125.55) | −69.93 (48.65) |
| Median | −38.46 | −26.00 | −89.45 | −100.00 |
| Q1; Q3 | −92.42; −9.90 | −70.89; −6.49 | −100.00; −36.00 | −100.00; −38.46 |
| Min; Max | −100.0; 50.0 | −100.0; 209.1 | −100.0; 541.7 | −100.0; 76.7 |
| Week 4 | | | | |
| Number (observed/imputed) | 24 (24/0) | 27 (26/1) | 25 (25/0) | 23 (23/0) |
| Mean (SD) | 36.02 (34.33) | 29.55 (20.68) | 14.79 (17.89) | 11.73 (16.82) |
| Median | 34.50 | 35.00 | 11.00 | 5.00 |
| Q1; Q3 | 0.58; 61.70 | 12.83; 45.00 | 0.00; 21.00 | 0.00; 18.00 |
| Min; Max | 0.0; 101.0 | 0.0; 66.0 | 0.0; 63.0 | 0.0; 62.0 |
| Change from baseline | | | | |
| Number (observed/imputed) | 24 (24/0) | 27 (26/1) | 25 (25/0) | 23 (23/0) |
| Mean (SD) | −22.15 (30.83) | −23.93 (24.26) | −30.05 (20.80) | −36.18 (24.55) |
| Median | −13.50 | −17.00 | −28.00 | −27.00 |
| Q1; Q3 | −44.50; 0.00 | −36.20; −4.00 | −43.00; −14.00 | −56.00; −16.00 |
| Min; Max | −91.0; 37.0 | −75.0; 5.0 | −79.0; 9.0 | −86.0; −2.0 |
| Percent change from baseline | | | | |
| Number (observed/imputed) | 24 (24/0) | 27 (26/1) | 25 (25/0) | 23 (23/0) |
| Mean (SD) | −31.17 (65.33) | −46.35 (39.61) | −53.69 (109.03) | −76.56 (30.09) |
| Median | −25.37 | −46.27 | −82.09 | −93.33 |
| Q1; Q3 | −99.14; 0.00 | −81.18; −5.97 | −100.00; −50.78 | −100.00; −63.33 |
| Min; Max | −100.0; 168.2 | −100.0; 26.3 | −100.0; 450.0 | −100.0; −4.0 |
| Week 5 | | | | |
| Number (observed/imputed) | 23 (23/0) | 26 (25/1) | 25 (24/1) | 23 (23/0) |
| Mean (SD) | 21.08 (24.61) | 24.55 (22.80) | 13.98 (20.55) | 10.83 (15.39) |
| Median | 12.83 | 23.50 | 0.00 | 6.00 |
| Q1; Q3 | 0.00; 35.00 | 0.00; 38.00 | 0.00; 20.00 | 0.00; 16.33 |
| Min; Max | 0.0; 98.0 | 0.0; 71.0 | 0.0; 64.0 | 0.0; 63.0 |
| Change from baseline | | | | |
| Number (observed/imputed) | 23 (23/0) | 26 (25/1) | 25 (24/1) | 23 (23/0) |
| Mean (SD) | −37.79 (31.17) | −29.64 (28.55) | −30.86 (24.37) | −37.08 (26.17) |
| Median | −41.00 | −17.00 | −24.00 | −30.00 |
| Q1; Q3 | −64.00; −12.00 | −55.00; −6.00 | −47.00; −13.00 | −59.00; −15.00 |
| Min; Max | −91.0; 36.3 | −80.0; 7.0 | −92.0; 2.7 | −91.0; 6.0 |
| Percent change from baseline | | | | |
| Number (observed/imputed) | 23 (23/0) | 26 (25/1) | 25 (24/1) | 23 (23/0) |
| Mean (SD) | −60.14 (58.83) | −55.69 (41.16) | −66.40 (54.16) | −73.55 (35.00) |
| Median | −81.13 | −59.46 | −100.00 | −88.24 |
| Q1; Q3 | −100.00; −52.17 | −100.00; −18.75 | −100.00; −33.11 | −100.00; −51.28 |
| Min; Max | −100.0; 165.2 | −100.0; 14.5 | −100.0; 133.3 | −100.0; 31.6 |
| Week 6 | | | | |
| Number (observed/imputed) | 22 (21/1) | 24 (23/1) | 25 (24/1) | 21 (21/0) |
| Mean (SD) | 25.43 (27.48) | 28.26 (23.00) | 13.26 (19.12) | 13.55 (20.58) |
| Median | 16.70 | 24.38 | 3.00 | 0.00 |
| Q1; Q3 | 0.00; 47.00 | 8.00; 46.50 | 0.00; 21.00 | 0.00; 16.00 |
| Min; Max | 0.0; 98.0 | 0.0; 69.0 | 0.0; 63.0 | 0.0; 63.0 |
| Change from baseline | | | | |
| Number (observed/imputed) | 22 (21/1) | 24 (23/1) | 25 (24/1) | 21 (21/0) |
| Mean (SD) | −33.30 (33.06) | −28.36 (28.62) | −31.58 (23.39) | −32.35 (30.51) |
| Median | −27.00 | −23.63 | −24.00 | −27.00 |
| Q1; Q3 | −55.60; −11.00 | −46.50; −7.17 | −43.00; −14.00 | −55.00; −11.00 |
| Min; Max | −98.0; 34.0 | −79.2; 14.8 | −92.0; 1.0 | −91.0; 26.8 |
| Percent change from baseline | | | | |
| Number (observed/imputed) | 22 (21/1) | 24 (23/1) | 25 (24/1) | 21 (21/0) |
| Mean (SD) | −53.45 (59.54) | −47.92 (45.31) | −70.72 (39.56) | −63.91 (58.28) |
| Median | −65.64 | −55.14 | −92.19 | −100.00 |
| Q1; Q3 | −100.00; −22.95 | −80.43; −19.31 | −100.00; −45.83 | −100.00; −52.38 |
| Min; Max | −100.0; 154.5 | −100.0; 78.1 | −100.0; 50.0 | −100.0; 100.0 |
| Week 7 | | | | |
| Number (observed/imputed) | 23 (22/1) | 23 (21/2) | 25 (24/1) | 20 (20/0) |
| Mean (SD) | 22.56 (25.96) | 31.88 (21.51) | 14.38 (19.63) | 12.43 (15.87) |
| Median | 16.80 | 30.00 | 4.67 | 2.50 |
| Q1; Q3 | 0.00; 38.00 | 14.00; 49.00 | 0.00; 22.00 | 0.00; 26.00 |
| Min; Max | 0.0; 98.0 | 0.0; 70.0 | 0.0; 63.0 | 0.0; 45.5 |
| Change from baseline | | | | |
| Number (observed/imputed) | 23 (22/1) | 23 (21/2) | 25 (24/1) | 20 (20/0) |
| Mean (SD) | −36.31 (32.62) | −26.95 (30.21) | −33.54 (24.64) | −32.23 (26.57) |
| Median | −37.00 | −28.00 | −30.00 | −27.00 |
| Q1; Q3 | −62.00; −8.00 | −55.00; −5.00 | −47.00; −15.00 | −51.00; −13.50 |
| Min; Max | −95.0; 32.8 | −75.0; 42.8 | −92.0; 1.0 | 85.0; 26.5 |
| Percent change from baseline | | | | |
| Number (observed/imputed) | 23 (22/1) | 23 (21/2) | 25 (24/1) | 20 (20/0) |
| Mean (SD) | −56.79 (58.52) | −33.18 (69.78) | −68.05 (40.33) | −66.41 (55.40) |
| Median | −78.72 | −50.00 | −88.60 | −93.41 |
| Q1; Q3 | −100.00; −38.71 | −76.00; −9.09 | −100.00; −38.67 | −100.00; −48.21 |
| Min; Max | −100.0; 149.2 | −100.0; 225.4 | −100.0; 50.0 | −100.0; 139.5 |
| Week 8 | | | | |
| Number (observed/imputed) | 23 (22/1) | 23 (20/3) | 24 (23/1) | 19 (19/0) |
| Mean (SD) | 25.96 (27.00) | 24.98 (23.29) | 9.97 (16.69) | 12.20 (13.05) |
| Median | 26.00 | 18.00 | 0.00 | 11.00 |
| Q1; Q3 | 0.00; 39.00 | 0.00; 44.00 | 0.00; 16.00 | 0.00; 22.17 |
| Min; Max | 0.0; 98.0 | 0.0; 70.0 | 0.0; 63.0 | 0.0; 40.6 |
| Change from baseline | | | | |
| Number (observed/imputed) | 23 (22/1) | 23 (20/3) | 24 (23/1) | 19 (19/0) |
| Mean (SD) | −32.91 (32.23) | −29.80 (30.38) | −36.21 (24.67) | −33.38 (24.94) |
| Median | −39.00 | −28.00 | −32.00 | −25.00 |
| Q1; Q3 | −61.00; −8.00 | −56.00; −7.00 | −52.50; −16.00 | −51.00; −13.00 |
| Min; Max | −83.0; 52.2 | −77.0; 42.8 | −92.0; 1.5 | −80.0; 5.6 |
| Percent change from baseline | | | | |
| Number (observed/imputed) | 23 (22/1) | 23 (20/3) | 24 (23/1) | 19 (19/0) |
| Mean (SD) | −49.15 (73.10) | −49.05 (70.23) | −74.90 (43.31) | −69.90 (38.13) |
| Median | −70.71 | −66.00 | −100.00 | −85.88 |
| Q1; Q3 | −100.00; −22.95 | −100.00; −11.94 | −100.00; −63.27 | −100.00; −44.00 |
| Min; Max | −100.0; 237.3 | −100.0; 225.4 | −100.0; 75.0 | −100.0; 23.8 |
| Week 9 | | | | |
| Number (observed/imputed) | 22 (21/1) | 24 (21/3) | 23 (22/1) | 20 (20/0) |
| Mean (SD) | 26.38 (25.55) | 27.96 (21.62) | 10.87 (18.30) | 8.52 (12.19) |
| Median | 29.58 | 26.30 | 0.00 | 1.40 |
| Q1; Q3 | 0.00; 40.00 | 9.50; 44.00 | 0.00; 12.00 | 0.00; 15.00 |
| Min; Max | 0.0; 98.0 | 0.0; 71.0 | 0.0; 63.0 | 0.0; 34.0 |
| Change from baseline | | | | |
| Number (observed/imputed) | 22 (21/1) | 24 (21/3) | 23 (22/1) | 20 (20/0) |
| Mean (SD) | −32.17 (30.60) | −28.66 (27.25) | −34.83 (26.07) | −38.54 (26.30) |
| Median | −38.50 | −29.00 | −32.00 | −39.00 |
| Q1; Q3 | −58.80; −3.00 | −52.50; −7.50 | −46.00; −16.00 | −55.00; −17.00 |
| Min; Max | −84.0; 22.3 | −75.0; 42.8 | −92.0; 4.0 | −85.0; 3.5 |
| Percent change from baseline | | | | |
| Number (observed/imputed) | 22 (21/1) | 24 (21/3) | 23 (22/1) | 20 (20/0) |
| Mean (SD) | −47.50 (57.76) | −46.39 (66.49) | −67.10 (66.88) | −78.96 (34.18) |
| Median | −62.54 | −56.26 | −100.00 | −98.13 |
| Q1; Q3 | −100.00; −4.41 | −83.27; −33.38 | −100.00; −58.21 | −100.00; −65.23 |
| Min; Max | −100.0; 101.5 | −100.0; 225.4 | −100.0; 200.0 | −100.0; 16.7 |
| Week 10 | | | | |
| Number (observed/imputed) | 23 (22/1) | 24 (21/3) | 21 (20/1) | 21 (21/0) |
| Mean (SD) | 26.17 (27.80) | 27.70 (22.96) | 13.93 (20.43) | 6.83 (9.73) |
| Median | 21.00 | 28.00 | 0.00 | 2.00 |
| Q1; Q3 | 0.00; 41.00 | 0.00; 48.00 | 0.00; 24.00 | 0.00; 11.00 |
| Min; Max | 0.0; 98.0 | 0.0; 70.0 | 0.0; 63.0 | 0.0; 32.0 |
| Change from baseline | | | | |
| Number (observed/imputed) | 23 (22/1) | 24 (21/3) | 21 (20/1) | 21 (21/0) |
| Mean (SD) | −32.70 (32.03) | −28.93 (28.14) | −29.84 (25.74) | −39.26 (24.84) |
| Median | −30.00 | −28.92 | −24.00 | −40.00 |
| Q1; Q3 | −62.83; −8.00 | −50.00; −9.00 | −38.00; −14.00 | −56.00; −18.00 |
| Min; Max | −91.0; 25.8 | −75.0; 42.8 | −92.0; 10.0 | −85.0; 7.0 |
| Percent change from baseline | | | | |
| Number (observed/imputed) | 23 (22/1) | 24 (21/3) | 21 (20/1) | 21 (21/0) |
| Mean (SD) | −48.53 (61.08) | −46.90 (68.16) | −45.78 (130.13) | −82.44 (32.63) |
| Median | −64.49 | −50.17 | −100.00 | −96.61 |
| Q1; Q3 | −100.00; −15.12 | −100.00; −31.94 | −100.00; −34.86 | −100.00; −80.65 |
| Min; Max | −100.0; 117.4 | −100.0; 225.4 | −100.0; 500.0 | −100.0; 33.3 |
| Week 11 | | | | |
| Number (observed/imputed) | 23 (22/1) | 24 (21/3) | 21 (20/1) | 21 (21/0) |
| Mean (SD) | 21.57 (26.48) | 27.44 (24.07) | 11.30 (19.07) | 9.80 (13.81) |
| Median | 10.50 | 30.10 | 0.00 | 3.00 |
| Q1; Q3 | 0.00; 35.00 | 0.00; 46.67 | 0.00; 23.00 | 0.00; 19.60 |
| Min; Max | 0.0; 98.0 | 0.0; 70.0 | 0.0; 63.0 | 0.0; 46.0 |
| Change from baseline | | | | |
| Number (observed/imputed) | 23 (22/1) | 24 (21/3) | 21 (20/1) | 21 (21/0) |
| Mean (SD) | −37.30 (31.67) | −29.18 (28.09) | −35.05 (24.68) | −36.30 (27.49) |
| Median | −41.17 | −32.40 | −29.17 | −31.00 |
| Q1; Q3 | −60.00; −8.00 | −51.40; −7.00 | −47.00; −16.00 | −55.00; −11.17 |
| Min; Max | −99.0; 16.0 | −75.0; 42.8 | −92.0; 2.0 | −91.0; −1.4 |
| Percent change from baseline | | | | |
| Number (observed/imputed) | 23 (22/1) | 24 (21/3) | 21 (20/1) | 21 (21/0) |
| Mean (SD) | −58.67 (50.47) | −47.96 (69.01) | −73.13 (48.67) | −76.08 (33.07) |
| Median | −71.15 | −52.90 | −100.00 | −96.00 |
| Q1; Q3 | −100.00; −26.23 | −100.00; −19.25 | −100.00; −65.67 | −100.00; −65.69 |
| Min; Max | −100.0; 94.1 | −100.0; 225.4 | −100.0; 100.0 | −100.0; −4.0 |
| Week 12 | | | | |
| Number (observed/imputed) | 22 (21/1) | 21 (18/3) | 20 (18/2) | 20 (19/1) |
| Mean (SD) | 21.96 (24.66) | 25.87 (23.04) | 11.73 (18.07) | 7.10 (12.86) |
| Median | 18.08 | 26.83 | 0.00 | 0.00 |
| Q1; Q3 | 0.00; 33.83 | 0.00; 43.40 | 0.00; 21.40 | 0.00; 9.33 |
| Min; Max | 0.0; 95.7 | 0.0; 71.2 | 0.0; 63.0 | 0.0; 37.0 |
| Change from baseline | | | | |
| Number (observed/imputed) | 22 (21/1) | 21 (18/3) | 20 (18/2) | 20 (19/1) |
| Mean (SD) | −36.77 (31.45) | −29.03 (28.74) | −35.73 (26.59) | −37.55 (25.86) |
| Median | −40.00 | −28.00 | −37.00 | −31.00 |
| Q1; Q3 | −59.00; −8.00 | −56.00; −8.00 | −49.60; −15.00 | −57.00; −16.00 |
| Min; Max | −91.0; 11.8 | −75.0; 42.8 | −92.0; 7.0 | −91.0; −3.0 |
| Percent change from baseline | | | | |
| Number (observed/imputed) | 22 (21/1) | 21 (18/3) | 20 (18/2) | 20 (19/1) |
| Mean (SD) | −56.70 (50.79) | −48.34 (71.54) | −76.62 (36.34) | −82.72 (30.20) |
| Median | −68.13 | −59.34 | −100.00 | −100.00 |
| Q1; Q3 | −100.00; −32.79 | −100.00; −25.12 | −100.00; −62.99 | −100.00; −66.28 |
| Min; Max | −100.0; 64.7 | −100.0; 225.4 | −100.0; 30.4 | −100.0; −7.7 |
| End of Study | | | | |
| Number (observed/imputed) | 4 (2/2) | 4 (1/3) | 3 (1/2) | 4 (3/1) |
| Mean (SD) | 33.50 (31.96) | 17.46 (29.82) | 27.33 (24.01) | 26.75 (33.68) |
| Median | 28.50 | 4.00 | 37.00 | 16.00 |
| Q1; Q3 | 14.00; 53.00 | 0.00; 34.92 | 0.00; 45.00 | 4.00; 49.50 |
| Min; Max | 0.0; 77.0 | 0.0; 61.8 | 0.0; 45.0 | 0.0; 75.0 |
| Change from baseline | | | | |
| Number (observed/imputed) | 4 (2/2) | 4 (1/3) | 3 (1/2) | 4 (3/1) |
| Mean (SD | −23.75 (27.40) | −4.04 (35.07) | −38.33 (25.03) | −21.25 (14.29) |
| Median | −22.00 | −8.50 | −37.00 | −27.00 |
| Q1; Q3 | −44.00; −3.50 | −26.50; 18.42 | −64.00; −14.00 | −29.00; −13.50 |
| Min; Max | −58.0; 7.0 | −42.0; 42.8 | −64.0; −14.0 | −31.0; 0.0 |
| Percent change from baseline | | | | |
| Number (observed/imputed) | 4 (2/2) | 4 (1/3) | 3 (1/2) | 4 (3/1) |
| Mean (SD) | −37.75 (58.01) | −14.64 (160.23) | −57.91 (38.75) | −58.11 (43.26) |
| Median | −41.41 | −92.00 | −50.00 | −66.21 |
| Q1; Q3 | −83.72; 8.22 | −100.00; 70.72 | −100.00; −23.73 | −89.74; −26.47 |
| Min; Max | −100.0; 31.8 | −100.0; 225.4 | −100.0; −23.7 | −100.0; 0.0 |
| | Patients with Prior Omalizumab Use, Including Omalizumab- | | | |
| | Incomplete Responders | | | |
| | | Rilzabrutinib | Rilzabrutinib | Rilzabrutinib |
| | Placebo | 400 mg QPM | 400 mg BID | 400 mg TID |
| AAS7 | (N = 4) | (N = 1) | (N = 6) | (N = 6) |
| Baseline | | | | |
| Number | 4 | 1 | 2 | 3 |
| Mean (SD) | 66.00 (34.21) | 68.00 (NC) | 39.50 (3.54) | 31.28 (12.60) |
| Median | 68.50 | 68.00 | 39.50 | 36.00 |
| Q1; Q3 | 37.00; 95.00 | 68.00; 68.00 | 37.00; 42.00 | 17.00; 40.83 |
| Min; Max | 29.0; 98.0 | 68.0; 68.0 | 37.0; 42.0 | 17.0; 40.8 |
| Week 1 | | | | |
| Number (observed/imputed) | 4 (4/0) | 1 (1/0) | 2 (2/0) | 3 (3/0) |
| Mean (SD) | 37.96 (36.04) | 27.00 (NC) | 0.00 (0.00) | 6.33 (7.77) |
| Median | 31.00 | 27.00 | 0.00 | 4.00 |
| Q1; Q3 | 9.42; 66.50 | 27.00; 27.00 | 0.00; 0.00 | 0.00; 15.00 |
| Min; Max | 5.8; 84.0 | 27.0; 27.0 | 0.0; 0.0 | 0.0; 15.0 |
| Change from baseline | | | | |
| Number (observed/imputed) | 4 (4/0) | 1 (1/0) | 2 (2/0) | 3 (3/0) |
| Mean (SD) | −28.04 (12.39) | 41.00 (NC) | −39.50 (3.54) | −24.94 (11.53) |
| Median | −27.58 | −41.00 | −39.50 | −25.83 |
| Q1; Q3 | −37.50; −18.58 | −41.00; −41.00 | −42.00; −37.00 | −36.00; −13.00 |
| Min; Max | −43.0; −14.0 | −41.0; −41.0 | −42.0; −37.0 | −36.0; −13.0 |
| Percent change from baseline | | | | |
| Number (observed/imputed) | 4 (4/0) | 1 (1/0) | 2 (2/0) | 3 (3/0) |
| Mean (SD) | −53.01 (29.38) | −60.29 (NC) | −100.00 (0.00) | −79.91 (18.61) |
| Median | −58.93 | −60.29 | −100.00 | −76.47 |
| Q1; Q3 | −75.50; −30.51 | −60.29; −60.29 | −100.00; −100.00 | −100.00; −63.27 |
| Min; Max | −79.9; −14.3 | −60.3; −60.3 | −100.0; −100.0 | −100.0; −63.3 |
| Week 2 | | | | |
| Number (observed/imputed) | 4 (4/0) | 1 (1/0) | 2 (2/0) | 3 (3/0) |
| Mean (SD) | 21.21 (38.63) | 25.00 (NC) | 0.00 (0.00) | 4.00 (6.93) |
| Median | 2.92 | 25.00 | 0.00 | 0.00 |
| Q1; Q3 | 0.00; 42.42 | 25.00; 25.00 | 0.00; 0.00 | 0.00; 12.00 |
| Min; Max | 0.0; 79.0 | 25.0; 25.0 | 0.0; 0.0 | 0.0; 12.0 |
| Change from baseline | | | | |
| Number (observed/imputed) | 4 (4/0) | 1 (1/0) | 2 (2/0) | 3 (3/0) |
| Mean (SD) | −44.79 (33.47) | −43.00 (NC) | −39.50 (3.54) | −27.28 (19.44) |
| Median | −34.08 | −43.00 | −39.50 | −36.00 |
| Q1; Q3 | −68.50; −21.08 | −43.00; −43.00 | −42.00; −37.00 | −40.83; −5.00 |
| Min; Max | −92.0; −19.0 | −43.0; −43.0 | −42.0; −37.0 | −40.8; −5.0 |
| Percent change from baseline | | | | |
| Number (observed/imputed) | 4 (4/0) | 1 (1/0) | 2 (2/0) | 3 (3/0) |
| Mean (SD) | −74.82 (38.15) | −63.24 (NC) | −100.00 (0.00) | −76.47 (40.75) |
| Median | −89.94 | −63.24 | −100.00 | −100.00 |
| Q1; Q3 | −100.00; −49.64 | −63.24; −63.24 | −100.00; −100.00 | −100.00; −29.41 |
| Min; Max | −100.0; −19.4 | −63.2; −63.2 | −100.0; −100.0 | −100.0; −29.4 |
| Week 3 | | | | |
| Number (observed/imputed) | 4 (4/0) | 1 (1/0) | 2 (2/0) | 3 (3/0) |
| Mean (SD) | 24.15 (36.42) | 29.00 (NC) | 0.00 (0.00) | 7.00 (7.00) |
| Median | 9.80 | 29.00 | 0.00 | 7.00 |
| Q1; Q3 | 0.00; 48.30 | 29.00; 29.00 | 0.00; 0.00 | 0.00; 14.00 |
| Min; Max | 0.0; 77.0 | 29.0; 29.0 | 0.0; 0.0 | 0.0; 14.0 |
| Change from baseline | | | | |
| Number (observed/imputed) | 4 (4/0) | 1 (1/0) | 2 (2/0) | 3 (3/0) |
| Mean (SD) | −41.85 (36.57) | −39.00 (NC) | −39.50 (3.54) | −24.28 (18.46) |
| Median | −33.00 | −39.00 | −39.50 | −33.83 |
| Q1; Q3 | −68.50; −15.20 | −39.00; −39.00 | −42.00; −37.00 | −36.00; −3.00 |
| Min; Max | −92.0; −9.4 | −39.0; −39.0 | −42.0; −37.0 | −36.0; −3.0 |
| Percent change from baseline | | | | |
| Number (observed/imputed) | 4 (4/0) | 1 (1/0) | 2 (2/0) | 3 (3/0) |
| Mean (SD) | −63.46 (42.43) | −57.35 (NC) | −100.00 (0.00) | −66.83 (43.45) |
| Median | −66.21 | −57.35 | −100.00 | −82.86 |
| Q1; Q3 | −100.00; −26.92 | −57.35; −57.35 | −100.00; −100.00 | −100.00; −17.65 |
| Min; Max | −100.0; −21.4 | −57.4; −57.4 | −100.0; −100.0 | −100.0; −17.6 |
| Week 4 | | | | |
| Number (observed/imputed) | 4 (4/0) | 1 (1/0) | 2 (2/0) | 3 (3/0) |
| Mean (SD) | 16.25 (32.50) | 26.00 (NC) | 0.00 (0.00) | 7.11 (6.30) |
| Median | 0.00 | 26.00 | 0.00 | 9.33 |
| Q1; Q3 | 0.00; 32.50 | 26.00; 26.00 | 0.00; 0.00 | 0.00; 12.00 |
| Min; Max | 0.0; 65.0 | 26.0; 26.0 | 0.0; 0.0 | 0.0; 12.0 |
| Change from baseline | | | | |
| Number (observed/imputed) | 4 (4/0) | 1 (1/0) | 2 (2/0) | 3 (3/0) |
| Mean (SD) | −49.75 (28.98) | −42.00 (NC) | −39.50 (3.54) | −24.17 (16.75) |
| Median | −39.00 | −42.00 | −39.50 | −31.50 |
| Q1; Q3 | −68.50; −31.00 | −42.00; −42.00 | −42.00; −37.00 | −36.00; −5.00 |
| Min; Max | −92.0; −29.0 | −42.0; −42.0 | −42.0; −37.0 | −36.0; −5.0 |
| Percent change from baseline | | | | |
| Number (observed/imputed) | 4 (4/0) | 1 (1/0) | 2 (2/0) | 3 (3/0) |
| Mean (SD) | −83.42 (33.16) | −61.76 (NC) | −100.00 (0.00) | −68.85 (36.02) |
| Median | −100.00 | −61.76 | −100.00 | −77.14 |
| Q1; Q3 | −100.00; −66.84 | −61.76; −61.76 | −100.00; −100.00 | −100.00; −29.41 |
| Min; Max | −100.0; −33.7 | −61.8; −61.8 | −100.0; −100.0 | −100.0; −29.4 |
| Week 5 | | | | |
| Number (observed/imputed) | 4 (4/0) | 1 (1/0) | 2 (2/0) | 3 (3/0) |
| Mean (SD) | 31.88 (31.82) | 37.00 (NC) | 0.00 (0.00) | 13.28 (11.50) |
| Median | 27.00 | 37.00 | 0.00 | 19.83 |
| Q1; Q3 | 8.00; 55.75 | 37.00; 37.00 | 0.00; 0.00 | 0.00; 20.00 |
| Min; Max | 0.0; 73.5 | 37.0; 37.0 | 0.0; 0.0 | 0.0; 20.0 |
| Change from baseline | | | | |
| Number (observed/imputed) | 4 (4/0) | 1 (1/0) | 2 (2/0) | 3 (3/0) |
| Mean (SD) | −34.13 (18.73) | −31.00 (NC) | −39.50 (3.54) | −18.00 (19.67) |
| Median | −34.75 | −31.00 | −39.50 | −21.00 |
| Q1; Q3 | −49.50; −18.75 | −31.00; −31.00 | −42.00; −37.00 | −36.00; 3.00 |
| Min; Max | −54.0; −13.0 | −31.0; −31.0 | −42.0; −37.0 | −36.0; 3.0 |
| Percent change from baseline | | | | |
| Number (observed/imputed) | 4 (4/0) | 1 (1/0) | 2 (2/0) | 3 (3/0) |
| Mean (SD) | −57.13 (31.75) | −45.59 (NC) | −100.00 (0.00) | −44.59 (59.12) |
| Median | −51.76 | −45.59 | −100.00 | −51.43 |
| Q1; Q3 | −79.35; −34.91 | −45.59; −45.59 | −100.00; −100.00 | −100.00; 17.65 |
| Min; Max | −100.0; −25.0 | −45.6; −45.6 | −100.0; −100.0 | −100.0; 17.6 |
| Week 6 | | | | |
| Number (observed/imputed) | 4 (3/1) | 1 (1/0) | 2 (2/0) | 3 (3/0) |
| Mean (SD) | 31.83 (20.44) | 21.00 (NC) | 0.00 (0.00) | 7.00 (8.19) |
| Median | 30.17 | 21.00 | 0.00 | 5.00 |
| Q1; Q3 | 14.50; 49.17 | 21.00; 21.00 | 0.00; 0.00 | 0.00; 16.00 |
| Min; Max | 13.0; 54.0 | 21.0; 21.0 | 0.0; 0.0 | 0.0; 16.0 |
| Change from baseline | | | | |
| Number (observed/imputed) | 4 (3/1) | 1 (1/0) | 2 (2/0) | 3 (3/0) |
| Mean (SD) | −34.17 (16.81) | −47.00 (NC) | −39.50 (3.54) | −24.28 (20.16) |
| Median | −35.00 | −47.00 | −39.50 | −35.83 |
| Q1; Q3 | −45.83; −22.50 | −47.00; −47.00 | −42.00; −37.00 | −36.00; −1.00 |
| Min; Max | −53.7; −13.0 | −47.0; −47.0 | −42.0; −37.0 | −36.0; −1.0 |
| Percent change from baseline | | | | |
| Number (observed/imputed) | 4 (3/1) | 1 (1/0) | 2 (2/0) | 3 (3/0) |
| Mean (SD) | −53.00 (13.35) | −69.12 (NC) | −100.00 (0.00) | −64.55 (51.17) |
| Median | −49.79 | −69.12 | −100.00 | −87.76 |
| Q1; Q3 | −62.94; −43.07 | −69.12; −69.12 | −100.00; −100.00 | −100.00; −5.88 |
| Min; Max | −71.1; −41.3 | −69.1; −69.1 | −100.0; −100.0 | −100.0; −5.9 |
| Week 7 | | | | |
| Number (observed/imputed) | 4 (3/1) | 1 (1/0) | 2 (2/0) | 3 (3/0) |
| Mean (SD) | 37.90 (31.72) | 12.00 (NC) | 1.00 (1.41) | 9.72 (16.84) |
| Median | 36.60 | 12.00 | 1.00 | 0.00 |
| Q1; Q3 | 10.70; 65.10 | 12.00; 12.00 | 0.00; 2.00 | 0.00; 29.17 |
| Min; Max | 8.4; 70.0 | 12.0; 12.0 | 0.0; 2.0 | 0.0; 29.2 |
| Change from baseline | | | | |
| Number (observed/imputed) | 4 (3/1) | 1 (1/0) | 2 (2/0) | 3 (3/0) |
| Mean (SD) | −28.10 (8.22) | −56.00 (NC) | −38.50 (2.12) | −21.56 (12.79) |
| Median | −27.00 | −56.00 | −38.50 | −17.00 |
| Q1; Q3 | −34.90; −21.30 | −56.00; −56.00 | −40.00; −37.00 | −36.00; −11.67 |
| Min; Max | −37.8; −20.6 | −56.0; −56.0 | −40.0; −37.0 | −36.0; −11.7 |
| Percent change from baseline | | | | |
| Number (observed/imputed) | 4 (3/1) | 1 (1/0) | 2 (2/0) | 3 (3/0) |
| Mean (SD) | −51.16 (23.76) | −82.35 (NC) | −97.62 (3.37) | −76.19 (41.24) |
| Median | −54.80 | −82.35 | −97.62 | −100.00 |
| Q1; Q3 | −71.07; −31.24 | −82.35; −82.35 | −100.00; −95.24 | −100.00; −28.57 |
| Min; Max | −71.1; −23.9 | −82.4; −82.4 | −100.0; −95.2 | −100.0; −28.6 |
| Week 8 | | | | |
| Number (observed/imputed) | 4 (3/1) | 1 (1/0) | 2 (2/0) | 3 (3/0) |
| Mean (SD) | 25.25 (22.35) | 18.00 (NC) | 0.00 (0.00) | 4.67 (7.23) |
| Median | 27.50 | 18.00 | 0.00 | 1.00 |
| Q1; Q3 | 6.50; 44.00 | 18.00; 18.00 | 0.00; 0.00 | 0.00; 13.00 |
| Min; Max | 0.0; 46.0 | 18.0; 18.0 | 0.0; 0.0 | 0.0; 13.0 |
| Change from baseline | | | | |
| Number (observed/imputed) | 4 (3/1) | 1 (1/0) | 2 (2/0) | 3 (3/0) |
| Mean (SD) | −40.75 (12.58) | −50.00 (NC) | −39.50 (3.54) | −26.61 (19.68) |
| Median | −39.00 | −50.00 | −39.50 | −36.00 |
| Q1; Q3 | −51.00; −30.50 | −50.00; −50.00 | −42.00; −37.00 | −39.83; −4.00 |
| Min; Max | −56.0; −29.0 | −50.0; −50.0 | −42.0; −37.0 | −39.8; −4.0 |
| Percent change from baseline | | | | |
| Number (observed/imputed) | 4 (3/1) | 1 (1/0) | 2 (2/0) | 3 (3/0) |
| Mean (SD) | −69.56 (22.10) | −73.53 (NC) | −100.00 (0.00) | −73.69 (43.46) |
| Median | −64.13 | −73.53 | −100.00 | −97.55 |
| Q1; Q3 | −85.56; −53.57 | −73.53; −73.53 | −100.00; − | −100.00; −23.53 |
| Min; Max | −100.0; −50.0 | −73.5; −73.5 | −100.0; −100.0 | −100.0; −23.5 |
| Week 9 | | | | |
| Number (observed/imputed) | 4 (3/1) | 1 (1/0) | 2 (2/0) | 3 (3/0) |
| Mean (SD) | 16.03 (5.68) | 10.00 (NC) | 0.00 (0.00) | 4.39 (6.62) |
| Median | 13.50 | 10.00 | 0.00 | 1.17 |
| Q1; Q3 | 12.80; 19.25 | 10.00; 10.00 | 0.00; 0.00 | 0.00; 12.00 |
| Min; Max | 12.6; 24.5 | 10.0; 10.0 | 0.0; 0.0 | 0.0; 12.0 |
| Change from baseline | | | | |
| Number (observed/imputed) | 4 (3/1) | 1 (1/0) | 2 (2/0) | 3 (3/0) |
| Mean (SD) | −49.98 (31.17) | −58.00 (NC) | −39.50 (3.54) | −26.89 (19.04) |
| Median | −49.75 | −58.00 | −39.50 | −36.00 |
| Q1; Q3 | −75.75; −24.20 | −58.00; −58.00 | −42.00; −37.00 | −39.67; −5.00 |
| Min; Max | −84.0; −16.4 | −58.0; −58.0 | −42.0; −37.0 | −39.7; −5.0 |
| Percent change from baseline | | | | |
| Number (observed/imputed) | 4 (3/1) | 1 (1/0) | 2 (2/0) | 3 (3/0) |
| Mean (SD) | −71.69 (11.96) | −85.29 (NC) | −100.00 (0.00) | −75.52 (39.95) |
| Median | −72.24 | −85.29 | −100.00 | −97.14 |
| Q1; Q3 | −79.54; −63.83 | −85.29; −85.29 | −100.00; −100.00 | −100.00; −29.41 |
| Min; Max | −85.7; −56.6 | −85.3; −85.3 | −100.0; −100.0 | −100.0; −29.4 |
| Week 10 | | | | |
| Number (observed/imputed) | 4 (3/1) | 1 (1/0) | 2 (2/0) | 3 (2/1) |
| Mean (SD) | 7.50 (5.57) | 0.00 (NC) | 0.00 (0.00) | 9.00 (10.15) |
| Median | 8.50 | 0.00 | 0.00 | 7.00 |
| Q1; Q3 | 3.50; 11.50 | 0.00; 0.00 | 0.00; 0.00 | 0.00; 20.00 |
| Min; Max | 0.0; 13.0 | 0.0; 0.0 | 0.0; 0.0 | 0.0; 20.0 |
| Change from baseline | | | | |
| Number (observed/imputed) | 4 (3/1) | 1 (1/0) | 2 (2/0) | 3 (2/1) |
| Mean (SD) | −58.50 (37.18) | −68.00 (NC) | −39.50 (3.54) | −22.28 (21.92) |
| Median | −57.00 | −68.00 | −39.50 | −33.83 |
| Q1; Q3 | −90.00; −27.00 | −68.00; −68.00 | −42.00; −37.00 | −36.00; 3.00 |
| Min; Max | −98.0; −22.0 | −68.0; −68.0 | −42.0; −37.0 | −36.0; 3.0 |
| Percent change from baseline | | | | |
| Number (observed/imputed) | 4 (3/1) | 1 (1/0) | 2 (2/0) | 3 (2/1) |
| Mean (SD) | −84.03 (13.10) | −100.00 (NC) | −100.00 (0.00) | −55.07 (63.56) |
| Median | −82.50 | −100.00 | −100.00 | −82.86 |
| Q1; Q3 | −94.57; −73.49 | −100.00; −100.00 | −100.00; −100.00 | −100.00; 17.65 |
| Min; Max | −100.0; −71.1 | −100.0; −100.0 | −100.0; −100.0 | −100.0; 17.6 |
| Week 11 | | | | |
| Number (observed/imputed) | 4 (3/1) | 1 (1/0) | 2 (2/0) | 3 (2/1) |
| Mean (SD) | 8.96 (4.12) | 0.00 (NC) | 0.00 (0.00) | 6.67 (11.55) |
| Median | 8.92 | 0.00 | 0.00 | 0.00 |
| Q1; Q3 | 5.42; 12.50 | 0.00; 0.00 | 0.00; 0.00 | 0.00; 20.00 |
| Min; Max | 5.0; 13.0 | 0.0; 0.0 | 0.0; 0.0 | 0.0; 20.0 |
| Change from baseline | | | | |
| Number (observed/imputed) | 4 (3/1) | 1 (1/0) | 2 (2/0) | 3 (2/1) |
| Mean (SD) | −57.04 (34.06) | −68.00 (NC) | −39.50 (3.54) | −24.61 (24.03) |
| Median | −56.00 | −68.00 | −39.50 | −36.00 |
| Q1; Q3 | −86.08; −28.00 | −68.00; −68.00 | −42.00; −37.00 | −40.83; 3.00 |
| Min; Max | −92.2; −24.0 | −68.0; −68.0 | −42.0; −37.0 | −40.8; 3.0 |
| Percent change from baseline | | | | |
| Number (observed/imputed) | 4 (3/1) | 1 (1/0) | 2 (2/0) | 3 (2/1) |
| Mean (SD) | −83.72 (9.61) | −100.00 (NC) | −100.00 (0.00) | −60.78 (67.92) |
| Median | −84.86 | −100.00 | −100.00 | −100.00 |
| Q1; Q3 | −90.50; −76.93 | −100.00; −100.00 | −100.00; −100.00 | −100.00; 17.65 |
| Min; Max | −94.0; −71.1 | −100.0; −100.0 | −100.0; −100.0 | −100.0; 17.6 |
| Week 12 | | | | |
| Number (observed/imputed) | 3 (2/1) | 1 (1/0) | 2 (2/0) | 2 (1/1) |
| Mean (SD) | 6.67 (6.51) | 5.00 (NC) | 0.00 (0.00) | 10.00 (14.14) |
| Median | 7.00 | 5.00 | 0.00 | 10.00 |
| Q1; Q3 | 0.00; 13.00 | 5.00; 5.00 | 0.00; 0.00 | 0.00; 20.00 |
| Min; Max | 0.0; 13.0 | 5.0; 5.0 | 0.0; 0.0 | 0.0; 20.0 |
| Change from baseline | | | | |
| Number (observed/imputed) | 3 (2/1) | 1 (1/0) | 2 (2/0) | 2 (1/1) |
| Mean (SD) | −71.67 (34.96) | −63.00 (NC) | −39.50 (3.54) | −16.50 (27.58) |
| Median | −85.00 | −63.00 | −39.50 | −16.50 |
| Q1; Q3 | 98.00; −32.00 | −63.00; −63.00 | −42.00; −37.00 | −36.00; 3.00 |
| Min; Max | −98.0; −32.0 | −63.0; −63.0 | −42.0; −37.0 | −36.0; 3.0 |
| Percent change from baseline | | | | |
| Number (observed/imputed) | 3 (2/1) | 1 (1/0) | 2 (2/0) | 2 (1/1) |
| Mean (SD) | −87.83 (14.97) | −92.65 (NC) | −100.00 (0.00) | −41.18 (83.19) |
| Median | −92.39 | −92.65 | −100.00 | −41.18 |
| Q1; Q3 | −100.00; −71.11 | −92.65; −92.65 | −100.00; −100.00 | −100.00; 17.65 |
| Min; Max | −100.0; −71.1 | −92.6; −92.6 | −100.0; −100.0 | −100.0; 17.6 |
| End of Study | | | | |
| Number (observed/imputed) | 0 (0/0) | 0 (0/0) | 0 (0/0) | 2 (0/2) |
| Mean (SD) | | | | 30.42 (14.73) |
| Median | | | | 30.42 |
| Q1; Q3 | | | | 20.00; 40.83 |
| Min; Max | | | | 20.0; 40.8 |
| Change from baseline | | | | |
| Number (observed/imputed) | 0 (0/0) | 0 (0/0) | 0 (0/0) | 2 (0/2) |
| Mean (SD | | | | 1.50 (2.12) |
| Median | | | | 1.50 |
| Q1; Q3 | | | | 0.00; 3.00 |
| Min; Max | | | | 0.0; 3.0 |
| Percent change from baseline | | | | |
| Number (observed/imputed) | 0 (0/0) | 0 (0/0) | 0 (0/0) | 2 (0/2) |
| Mean (SD) | | | | 8.82 (12.48) |
| Median | | | | 8.82 |
| Q1; Q3 | | | | 0.00; 17.65 |
| Min; Max | | | | 0.0; 17.6 |

TABLE 41
Change and Percent Change from Baseline in AAS7 over Time up to Week
12 in Participants with Angioedema at Baseline (ITT Population)

| | | | | | Change difference for |
|---|---|---|---|---|---|
| | | | Post- | Change from | rilzabrutinib vs. placebo |
| | | Baseline | baseline | baseline | LS Mean |
| | n | Mean (SD) | Mean (SD) | LS Mean (SE)^{a} | (95% CI)^{a} |
| Week 1 | | | | | |
| Rilzabrutinib 400 mg TID | 26 | 45.99 (22.33) | 17.50 (22.29) | −29.55 (4.16) | −15.05 (−28.44, −1.67) |
| (N = 41) | | | | | |
| Rilzabrutinib 400 mg BID | 28 | 46.08 (25.73) | 23.35 (30.86) | −24.34 (4.19) | −9.84 (−22.84, 3.16) |
| (N = 41) | | | | | |
| Rilzabrutinib 400 mg QPM | 28 | 54.00 (25.23) | 34.57 (21.50) | −19.68 (3.99) | −5.18 (−18.04, 7.67) |
| (N = 38) | | | | | |
| Placebo (N = 40) | 30 | 59.70 (27.08) | 42.73 (29.16) | −14.50 (4.14) | |
| Week 2 | | | | | |
| Rilzabrutinib 400 mg TID | 26 | 45.99 (22.33) | 15.52 (20.15) | −32.35 (4.37) | −11.45 (−23.02, 0.12) |
| (N = 41) | | | | | |
| Rilzabrutinib 400 mg BID | 27 | 46.08 (25.73) | 22.75 (28.74) | −26.86 (4.43) | −5.96 (−17.22, 5.30) |
| (N = 41) | | | | | |
| Rilzabrutinib 400 mg QPM | 28 | 54.00 (25.23) | 32.95 (23.02) | −21.54 (4.19) | −0.63 (−11.74, 10.48) |
| (N = 38) | | | | | |
| Placebo (N = 40) | 29 | 59.70 (27.08) | 35.80 (30.53) | −20.90 (4.36) | |
| Week 3 | | | | | |
| Rilzabrutinib 400 mg TID | 26 | 45.99 (22.33) | 13.26 (21.30) | −36.15 (4.48) | −12.00 (−23.87, −0.13) |
| (N = 41) | | | | | |
| Rilzabrutinib 400 mg BID | 28 | 46.08 (25.73) | 14.81 (20.54) | −35.57 (4.51) | −11.42 (−22.95, 0.12) |
| (N = 41) | | | | | |
| Rilzabrutinib 400 mg QPM | 28 | 54.00 (25.23) | 32.48 (20.51) | −21.82 (4.29) | 2.33 (−9.09, 13.74) |
| (N = 38) | | | | | |
| Placebo (N = 40) | 29 | 59.70 (27.08) | 31.90 (31.01) | −24.15 (4.48) | |
| Week 4 | | | | | |
| Rilzabrutinib 400 mg TID | 26 | 45.99 (22.33) | 11.20 (15.95) | −38.27 (4.30) | −15.67 (−27.11, −4.22) |
| (N = 41) | | | | | |
| Rilzabrutinib 400 mg BID | 27 | 46.08 (25.73) | 13.70 (17.64) | −35.34 (4.34) | −12.73 (−23.88, −1.58) |
| (N = 41) | | | | | |
| Rilzabrutinib 400 mg QPM | 28 | 54.00 (25.23) | 29.42 (20.30) | −25.28 (4.12) | −2.68 (−13.70, 8.35) |
| (N = 38) | | | | | |
| Placebo (N = 40) | 28 | 59.70 (27.08) | 33.19 (34.22) | −22.60 (4.34) | |
| Week 5 | | | | | |
| Rilzabrutinib 400 mg TID | 26 | 45.99 (22.33) | 11.12 (14.82) | −38.91 (4.22) | −9.32 (−20.56, 1.93) |
| (N = 41) | | | | | |
| Rilzabrutinib 400 mg BID | 27 | 46.08 (25.73) | 12.95 (20.09) | −36.13 (4.29) | −6.53 (−17.51, 4.44) |
| (N = 41) | | | | | |
| Rilzabrutinib 400 mg QPM | 27 | 54.00 (25.23) | 25.01 (22.48) | −28.04 (4.08) | 1.56 (−9.31, 12.42) |
| (N = 38) | | | | | |
| Placebo (N = 40) | 27 | 59.70 (27.08) | 22.68 (25.39) | −29.59 (4.26) | |
| Week 6 | | | | | |
| Rilzabrutinib 400 mg TID | 24 | 45.99 (22.33) | 12.73 (19.47) | −38.03 (4.48) | −11.42 (−23.52, 0.68) |
| (N = 41) | | | | | |
| Rilzabrutinib 400 mg BID | 27 | 46.08 (25.73) | 12.28 (18.70) | −36.76 (4.50) | −10.15 (−21.84, 1.54) |
| (N = 41) | | | | | |
| Rilzabrutinib 400 mg QPM | 25 | 54.00 (25.23) | 27.97 (22.56) | −25.61 (4.32) | 1.00 (−10.55, 12.54) |
| (N = 38) | | | | | |
| Placebo (N = 40) | 26 | 59.70 (27.08) | 26.41 (26.26) | −26.61 (4.62) | |
| Week 7 | | | | | |
| Rilzabrutinib 400 mg TID | 23 | 45.99 (22.33) | 12.07 (15.62) | −37.16 (4.45) | −9.86 (−21.60, 1.88) |
| (N = 41) | | | | | |
| Rilzabrutinib 400 mg BID | 27 | 46.08 (25.73) | 13.39 (19.19) | −37.72 (4.37) | −10.42 (−21.71, 0.87) |
| (N = 41) | | | | | |
| Rilzabrutinib 400 mg QPM | 24 | 54.00 (25.23) | 31.05 (21.43) | −22.95 (4.29) | 4.35 (−7.06, 15.76) |
| (N = 38) | | | | | |
| Placebo (N = 40) | 27 | 59.70 (27.08) | 24.84 (26.78) | −27.30 (4.43) | |
| Week 8 | | | | | |
| Rilzabrutinib 400 mg TID | 22 | 45.99 (22.33) | 11.17 (12.56) | −39.35 (4.28) | −11.02 (−22.54, 0.50) |
| (N = 41) | | | | | |
| Rilzabrutinib 400 mg BID | 26 | 46.08 (25.73) | 9.20 (16.24) | −41.63 (4.21) | −13.31 (−24.25, −2.37) |
| (N = 41) | | | | | |
| Rilzabrutinib 400 mg QPM | 24 | 54.00 (25.23) | 24.69 (22.82) | −30.07 (4.07) | −1.74 (−12.55, 9.06) |
| (N = 38) | | | | | |
| Placebo (N = 40) | 27 | 59.70 (27.08) | 25.86 (25.97) | −28.33 (4.29) | |
| Week 9 | | | | | |
| Rilzabrutinib 400 mg TID | 23 | 45.99 (22.33) | 7.98 (11.59) | −42.57 (4.09) | −13.12 (−23.93, −2.31) |
| (N = 41) | | | | | |
| Rilzabrutinib 400 mg BID | 25 | 46.08 (25.73) | 10.00 (17.78) | −40.70 (4.08) | −11.25 (−21.79, −0.72) |
| (N = 41) | | | | | |
| Rilzabrutinib 400 mg QPM | 25 | 54.00 (25.23) | 27.24 (21.46) | −26.47 (3.93) | 2.98 (−7.44, 13.40) |
| (N = 38) | | | | | |
| Placebo (N = 40) | 26 | 59.70 (27.08) | 24.79 (23.80) | −29.45 (4.07) | |
| Week 10 | | | | | |
| Rilzabrutinib 400 mg TID | 24 | 45.99 (22.33) | 7.10 (9.58) | −43.40 (4.49) | −13.07 (−24.96, −1.18) |
| (N = 41) | | | | | |
| Rilzabrutinib 400 mg BID | 23 | 46.08 (25.73) | 12.72 (19.89) | −39.12 (4.47) | −8.79 (−20.40, 2.82) |
| (N = 41) | | | | | |
| Rilzabrutinib 400 mg QPM | 25 | 54.00 (25.23) | 26.59 (23.15) | −27.81 (4.28) | 2.53 (−8.76, 13.81) |
| (N = 38) | | | | | |
| Placebo (N = 40) | 27 | 59.70 (27.08) | 23.41 (26.52) | −30.33 (4.43) | |
| Week 11 | | | | | |
| Rilzabrutinib 400 mg TID | 24 | 45.99 (22.33) | 9.40 (13.36) | −40.74 (4.34) | −6.88 (−18.42, 4.67) |
| (N = 41) | | | | | |
| Rilzabrutinib 400 mg BID | 23 | 46.08 (25.73) | 10.31 (18.47) | −42.03 (4.41) | −8.17 (−19.55, 3.20) |
| (N = 41) | | | | | |
| Rilzabrutinib 400 mg QPM | 25 | 54.00 (25.23) | 26.34 (24.19) | −28.68 (4.16) | 5.18 (−6.05, 16.41) |
| (N = 38) | | | | | |
| Placebo (N = 40) | 27 | 59.70 (27.08) | 19.70 (24.82) | −33.86 (4.39) | |
| Week 12 | | | | | |
| Rilzabrutinib 400 mg TID | 22 | 45.99 (22.33) | 7.37 (12.64) | −43.36 (4.14) | −8.06 (−19.05, 2.94) |
| (N = 41) | | | | | |
| Rilzabrutinib 400 mg BID | 22 | 46.08 (25.73) | 10.67 (17.53) | −42.38 (4.13) | −7.07 (−17.69, 3.54) |
| (N = 41) | | | | | |
| Rilzabrutinib 400 mg QPM | 22 | 54.00 (25.23) | 24.92 (22.92) | −28.63 (4.01) | 6.68 (−4.00, 17.35) |
| (N = 38) | | | | | |
| Placebo (N = 40) | 25 | 59.70 (27.08) | 20.12 (23.69) | −35.31 (4.14) | |

| | | % Change | % Change difference for | |
|---|---|---|---|---|
| | Change difference for | from | rilzabrutinib vs. placebo | |
| | rilzabrutinib vs. placebo | baseline | LS Mean | |
| | p-value^{a} | LS Mean (SE)^{a} | (95% CI)^{a} | p-value^{a} |
| Week 1 | | | | |
| Rilzabrutinib 400 mg TID | 0.0085 | −54.51 (9.77) | −34.55 (−66.00, −3.09) | 0.0101 |
| (N = 41) | | | | |
| Rilzabrutinib 400 mg BID | 0.0737 | −60.34 (9.85) | −40.37 (−70.92, −9.83) | 0.0021 |
| (N = 41) | | | | |
| Rilzabrutinib 400 mg QPM | 0.3380 | −32.14 (9.37) | −12.18 (−42.38, 18.02) | 0.3380 |
| (N = 38) | | | | |
| Placebo (N = 40) | | −19.96 (9.73) | | |
| Week 2 | | | | |
| Rilzabrutinib 400 mg TID | 0.0524 | −60.12 (12.38) | −18.75 (−51.54, 14.04) | 0.2625 |
| (N = 41) | | | | |
| Rilzabrutinib 400 mg BID | 0.2996 | −49.52 (12.60) | −8.15 (−40.12, 23.82) | 0.6173 |
| (N = 41) | | | | |
| Rilzabrutinib 400 mg QPM | 0.9109 | −37.11 (11.87) | 4.26 (−27.23, 35.76) | 0.7908 |
| (N = 38) | | | | |
| Placebo (N = 40) | | −41.37 (12.36) | | |
| Week 3 | | | | |
| Rilzabrutinib 400 mg TID | 0.0476 | −73.45 (15.58) | −24.93 (−66.27, 16.41) | 0.2373 |
| (N = 41) | | | | |
| Rilzabrutinib 400 mg BID | 0.0524 | −55.85 (15.71) | −7.33 (−47.50, 32.83) | 0.7205 |
| (N = 41) | | | | |
| Rilzabrutinib 400 mg QPM | 0.6895 | −33.35 (14.94) | 15.17 (−24.59, 54.94) | 0.4545 |
| (N = 38) | | | | |
| Placebo (N = 40) | | −48.52 (15.62) | | |
| Week 4 | | | | |
| Rilzabrutinib 400 mg TID | 0.0073 | −78.35 (13.78) | −41.77 (−78.55, −4.98) | 0.0260 |
| (N = 41) | | | | |
| Rilzabrutinib 400 mg BID | 0.0252 | −60.60 (13.93) | −24.02 (−59.85, 11.81) | 0.1889 |
| (N = 41) | | | | |
| Rilzabrutinib 400 mg QPM | 0.6344 | −49.47 (13.21) | −12.88 (−48.34, 22.58) | 0.4765 |
| (N = 38) | | | | |
| Placebo (N = 40) | | −36.59 (13.98) | | |
| Week 5 | | | | |
| Rilzabrutinib 400 mg TID | 0.1043 | −69.87 (9.90) | −16.05 (−42.45, 10.36) | 0.2337 |
| (N = 41) | | | | |
| Rilzabrutinib 400 mg BID | 0.2432 | −68.19 (10.08) | −14.36 (−40.13, 11.41) | 0.2747 |
| (N = 41) | | | | |
| Rilzabrutinib 400 mg QPM | 0.7787 | −54.71 (9.56) | −0.88 (−26.37, 24.61) | 0.9461 |
| (N = 38) | | | | |
| Placebo (N = 40) | | −53.83 (10.03) | | |
| Week 6 | | | | |
| Rilzabrutinib 400 mg TID | 0.0644 | −64.66 (10.25) | −17.35 (−45.04, 10.34) | 0.2194 |
| (N = 41) | | | | |
| Rilzabrutinib 400 mg BID | 0.0887 | −70.86 (10.33) | −23.55 (−50.40, 3.31) | 0.0857 |
| (N = 41) | | | | |
| Rilzabrutinib 400 mg QPM | 0.8656 | −46.12 (9.92) | 1.19 (−25.36, 27.75) | 0.9297 |
| (N = 38) | | | | |
| Placebo (N = 40) | | −47.31 (10.61) | | |
| Week 7 | | | | |
| Rilzabrutinib 400 mg TID | 0.0996 | −68.52 (11.69) | −22.57 (−53.40, 8.26) | 0.1513 |
| (N = 41) | | | | |
| Rilzabrutinib 400 mg BID | 0.0705 | −71.37 (11.45) | −25.42 (−54.97, 4.13) | 0.0918 |
| (N = 41) | | | | |
| Rilzabrutinib 400 mg QPM | 0.4545 | −29.64 (11.30) | 16.32 (−13.52, 46.16) | 0.2838 |
| (N = 38) | | | | |
| Placebo (N = 40) | | −45.95 (11.59) | | |
| Week 8 | | | | |
| Rilzabrutinib 400 mg TID | 0.0608 | −74.30 (11.67) | −22.25 (−53.63, 9.12) | 0.1645 |
| (N = 41) | | | | |
| Rilzabrutinib 400 mg BID | 0.0171 | −81.40 (11.62) | −29.34 (−59.45, 0.76) | 0.0560 |
| (N = 41) | | | | |
| Rilzabrutinib 400 mg QPM | 0.7517 | −54.56 (11.16) | −2.51 (−32.23, 27.22) | 0.8687 |
| (N = 38) | | | | |
| Placebo (N = 40) | | −52.05 (11.75) | | |
| Week 9 | | | | |
| Rilzabrutinib 400 mg TID | 0.0174 | −84.57 (11.49) | −34.54 (−64.85, −4.23) | 0.0255 |
| (N = 41) | | | | |
| Rilzabrutinib 400 mg BID | 0.0363 | −76.91 (11.50) | −26.88 (−56.35, 2.58) | 0.0738 |
| (N = 41) | | | | |
| Rilzabrutinib 400 mg QPM | 0.5753 | −48.50 (10.95) | 1.53 (−27.52, 30.59) | 0.9177 |
| (N = 38) | | | | |
| Placebo (N = 40) | | −50.03 (11.36) | | |
| Week 10 | | | | |
| Rilzabrutinib 400 mg TID | 0.0313 | −87.33 (15.54) | −38.35 (−79.56, 2.86) | 0.0682 |
| (N = 41) | | | | |
| Rilzabrutinib 400 mg BID | 0.1378 | −64.07 (15.65) | −15.09 (−55.38, 25.21) | 0.4631 |
| (N = 41) | | | | |
| Rilzabrutinib 400 mg QPM | 0.6608 | −50.13 (14.94) | −1.14 (−41.08, 38.79) | 0.9553 |
| (N = 38) | | | | |
| Placebo (N = 40) | | −48.99 (15.58) | | |
| Week 11 | | | | |
| Rilzabrutinib 400 mg TID | 0.2430 | −76.91 (10.59) | −15.53 (−43.68, 12.62) | 0.2796 |
| (N = 41) | | | | |
| Rilzabrutinib 400 mg BID | 0.1591 | −84.81 (10.85) | −23.43 (−51.36, 4.50) | 0.1002 |
| (N = 41) | | | | |
| Rilzabrutinib 400 mg QPM | 0.3656 | −53.78 (10.24) | 7.61 (−19.90, 35.12) | 0.5877 |
| (N = 38) | | | | |
| Placebo (N = 40) | | −61.39 (10.72) | | |
| Week 12 | | | | |
| Rilzabrutinib 400 mg TID | 0.1511 | −82.82 (10.03) | −19.84 (−46.44, 6.76) | 0.1438 |
| (N = 41) | | | | |
| Rilzabrutinib 400 mg BID | 0.1916 | −87.51 (10.12) | −24.53 (−50.33, 1.27) | 0.0624 |
| (N = 41) | | | | |
| Rilzabrutinib 400 mg QPM | 0.2200 | −54.80 (9.75) | 8.18 (−17.80, 34.16) | 0.5371 |
| (N = 38) | | | | |
| Placebo (N = 40) | | −62.98 (10.04) | | |

^{a}Each of the imputed complete data were analyzed by fitting an ANCOVA model with the corresponding baseline value, intervention group and regions as covariates.

TABLE 42
Change and Percent Change from Baseline in AAS7 over Time up to Week 12 in Participants
with Angioedema at Baseline (Omalizumab-Naïve Population)

| | | | | | Change difference for |
|---|---|---|---|---|---|
| | | | Post- | Change from | rilzabrutinib vs. placebo |
| | | Baseline | baseline | baseline | LS Mean |
| | n | Mean (SD) | Mean (SD) | LS Mean (SE)^{a} | (95% CI)^{a} |
| Week 1 | | | | | |
| Rilzabrutinib 400 mg TID | 23 | 47.91 (22.78) | 18.96 (23.24) | −29.80 (4.48) | −16.03 (−30.69, −1.38) |
| (N = 35) | | | | | |
| Rilzabrutinib 400 mg BID | 26 | 46.58 (26.66) | 25.15 (31.33) | −23.46 (4.42) | −9.69 (−23.74, 4.37) |
| (N = 35) | | | | | |
| Rilzabrutinib 400 mg QPM | 27 | 53.48 (25.55) | 34.85 (21.86) | −19.33 (4.16) | −5.57 (−19.34, 8.21) |
| (N = 37) | | | | | |
| Placebo (N = 36) | 26 | 58.73 (26.52) | 43.46 (28.75) | −13.77 (4.48) | |
| Week 2 | | | | | |
| Rilzabrutinib 400 mg TID | 23 | 47.91 (22.78) | 17.03 (20.90) | −32.47 (4.53) | −13.79 (−25.99, −1.60) |
| (N = 35) | | | | | |
| Rilzabrutinib 400 mg BID | 25 | 46.58 (26.66) | 24.57 (29.12) | −26.11 (4.50) | −7.43 (−19.15, 4.29) |
| (N = 35) | | | | | |
| Rilzabrutinib 400 mg QPM | 27 | 53.48 (25.55) | 33.25 (23.40) | −21.21 (4.21) | −2.53 (−13.99, 8.94) |
| (N = 37) | | | | | |
| Placebo (N = 36) | 25 | 58.73 (26.52) | 38.14 (29.33) | −18.68 (4.55) | |
| Week 3 | | | | | |
| Rilzabrutinib 400 mg TID | 23 | 47.91 (22.78) | 14.08 (22.48) | −36.16 (4.71) | −13.01 (−25.71, −0.30) |
| (N = 35) | | | | | |
| Rilzabrutinib 400 mg BID | 26 | 46.58 (26.66) | 15.95 (20.90) | −35.08 (4.66) | −11.92 (−24.12, 0.27) |
| (N = 35) | | | | | |
| Rilzabrutinib 400 mg QPM | 27 | 53.48 (25.55) | 32.60 (20.89) | −21.85 (4.38) | 1.31 (−10.66, 13.27) |
| (N = 37) | | | | | |
| Placebo (N = 36) | 25 | 58.73 (26.52) | 33.14 (30.73) | −23.16 (4.75) | |
| Week 4 | | | | | |
| Rilzabrutinib 400 mg TID | 23 | 47.91 (22.78) | 11.73 (16.82) | −38.89 (4.51) | −19.05 (−31.29, −6.81) |
| (N = 35) | | | | | |
| Rilzabrutinib 400 mg BID | 25 | 46.58 (26.66) | 14.79 (17.89) | −34.90 (4.47) | −15.05 (−26.84, −3.27) |
| (N = 35) | | | | | |
| Rilzabrutinib 400 mg QPM | 27 | 53.48 (25.55) | 29.55 (20.68) | −25.16 (4.19) | −5.31 (−16.86, 6.24) |
| (N = 37) | | | | | |
| Placebo (N = 36) | 24 | 58.73 (26.52) | 36.02 (34.33) | −19.84 (4.60) | |
| Week 5 | | | | | |
| Rilzabrutinib 400 mg TID | 23 | 47.91 (22.78) | 10.83 (15.39) | −39.59 (4.46) | −8.94 (−21.03, 3.15) |
| (N = 35) | | | | | |
| Rilzabrutinib 400 mg BID | 25 | 46.58 (26.66) | 13.98 (20.55) | −35.28 (4.45) | −4.63 (−16.28, 7.02) |
| (N = 35) | | | | | |
| Rilzabrutinib 400 mg QPM | 26 | 53.48 (25.55) | 24.55 (22.80) | −28.57 (4.18) | 2.08 (−9.37, 13.52) |
| (N = 37) | | | | | |
| Placebo (N = 36) | 23 | 58.73 (26.52) | 21.08 (24.61) | −30.65 (4.54) | |
| Week 6 | | | | | |
| Rilzabrutinib 400 mg TID | 21 | 47.91 (22.78) | 13.55 (20.58) | −38.09 (4.88) | −10.77 (−24.18, 2.64) |
| (N = 35) | | | | | |
| Rilzabrutinib 400 mg BID | 25 | 46.58 (26.66) | 13.26 (19.12) | −36.17 (4.80) | −8.84 (−21.64, 3.95) |
| (N = 35) | | | | | |
| Rilzabrutinib 400 mg QPM | 24 | 53.48 (25.55) | 28.26 (23.00) | −25.45 (4.55) | 1.87 (−10.66, 14.40) |
| (N = 37) | | | | | |
| Placebo (N = 36) | 22 | 58.73 (26.52) | 25.43 (27.48) | −27.32 (5.07) | |
| Week 7 | | | | | |
| Rilzabrutinib 400 mg TID | 20 | 47.91 (22.78) | 12.43 (15.87) | −37.68 (4.70) | −8.96 (−21.57, 3.65) |
| (N = 35) | | | | | |
| Rilzabrutinib 400 mg BID | 25 | 46.58 (26.66) | 14.38 (19.63) | −37.12 (4.53) | −8.40 (−20.39, 3.59) |
| (N = 35) | | | | | |
| Rilzabrutinib 400 mg QPM | 23 | 53.48 (25.55) | 31.88 (21.51) | −22.00 (4.38) | 6.71 (−5.27, 18.69) |
| (N = 37) | | | | | |
| Placebo (N = 36) | 23 | 58.73 (26.52) | 22.56 (25.96) | −28.72 (4.72) | |
| Week 8 | | | | | |
| Rilzabrutinib 400 mg TID | 19 | 47.91 (22.78) | 12.20 (13.05) | −39.04 (4.68) | −11.12 (−23.92, 1.69) |
| (N = 35) | | | | | |
| Rilzabrutinib 400 mg BID | 24 | 46.58 (26.66) | 9.97 (16.69) | −41.30 (4.49) | −13.38 (−25.33, −1.44) |
| (N = 35) | | | | | |
| Rilzabrutinib 400 mg QPM | 23 | 53.48 (25.55) | 24.98 (23.29) | −29.76 (4.29) | −1.84 (−13.56, 9.89) |
| (N = 37) | | | | | |
| Placebo (N = 36) | 23 | 58.73 (26.52) | 25.96 (27.00) | −27.92 (4.70) | |
| Week 9 | | | | | |
| Rilzabrutinib 400 mg TID | 20 | 47.91 (22.78) | 8.52 (12.19) | −42.94 (4.44) | −15.14 (−27.06, −3.23) |
| (N = 35) | | | | | |
| Rilzabrutinib 400 mg BID | 23 | 46.58 (26.66) | 10.87 (18.30) | −40.15 (4.33) | −12.36 (−23.79, −0.92) |
| (N = 35) | | | | | |
| Rilzabrutinib 400 mg QPM | 24 | 53.48 (25.55) | 27.96 (21.62) | −25.79 (4.12) | 2.00 (−9.20, 13.21) |
| (N = 37) | | | | | |
| Placebo (N = 36) | 22 | 58.73 (26.52) | 26.38 (25.55) | −27.79 (4.43) | |
| Week 10 | | | | | |
| Rilzabrutinib 400 mg TID | 21 | 47.91 (22.78) | 6.83 (9.73) | −44.63 (4.75) | −17.05 (−29.81, −4.29) |
| (N = 35) | | | | | |
| Rilzabrutinib 400 mg BID | 21 | 46.58 (26.66) | 13.93 (20.43) | −38.01 (4.62) | −10.43 (−22.69, 1.83) |
| (N = 35) | | | | | |
| Rilzabrutinib 400 mg QPM | 24 | 53.48 (25.55) | 27.70 (22.96) | −26.74 (4.35) | 0.84 (−10.97, 12.66) |
| (N = 37) | | | | | |
| Placebo (N = 36) | 23 | 58.73 (26.52) | 26.17 (27.80) | −27.58 (4.71) | |
| Week 11 | | | | | |
| Rilzabrutinib 400 mg TID | 21 | 47.91 (22.78) | 9.80 (13.81) | −41.34 (4.63) | −9.39 (−21.92, 3.13) |
| (N = 35) | | | | | |
| Rilzabrutinib 400 mg BID | 21 | 46.58 (26.66) | 11.30 (19.07) | −41.52 (4.63) | −9.58 (−21.83, 2.67) |
| (N = 35) | | | | | |
| Rilzabrutinib 400 mg QPM | 24 | 53.48 (25.55) | 27.44 (24.07) | −27.59 (4.31) | 4.36 (−7.60, 16.33) |
| (N = 37) | | | | | |
| Placebo (N = 36) | 23 | 58.73 (26.52) | 21.57 (26.48) | −31.95 (4.74) | |
| Week 12 | | | | | |
| Rilzabrutinib 400 mg TID | 20 | 47.91 (22.78) | 7.10 (12.86) | −44.04 (4.42) | −10.65 (−22.56, 1.26) |
| (N = 35) | | | | | |
| Rilzabrutinib 400 mg BID | 20 | 46.58 (26.66) | 11.73 (18.07) | −41.53 (4.39) | −8.14 (−19.74, 3.45) |
| (N = 35) | | | | | |
| Rilzabrutinib 400 mg QPM | 21 | 53.48 (25.55) | 25.87 (23.04) | −26.98 (4.16) | 6.41 (−5.00, 17.81) |
| (N = 37) | | | | | |
| Placebo (N = 36) | 22 | 58.73 (26.52) | 21.96 (24.66) | −33.39 (4.44) | |

| | | | % Change difference for | |
|---|---|---|---|---|
| | Change difference for | % Change | rilzabrutinib vs. placebo | |
| | rilzabrutinib vs. placebo | from baseline | LS Mean | |
| | p-value^{a} | LS Mean (SE)^{a} | (95% CI)^{a} | p-value^{a} |
| Week 1 | | | | |
| Rilzabrutinib 400 mg TID | 0.0105 | −53.40 (10.59) | −36.35 (−71.01, −1.69) | 0.0140 |
| (N = 35) | | | | |
| Rilzabrutinib 400 mg BID | 0.1032 | −59.22 (10.46) | −42.17 (−75.41, −8.93) | 0.0032 |
| (N = 35) | | | | |
| Rilzabrutinib 400 mg QPM | 0.3371 | −31.74 (9.84) | −14.70 (−47.27, 17.88) | 0.2843 |
| (N = 37) | | | | |
| Placebo (N = 36) | | −17.04 (10.60) | | |
| Week 2 | | | | |
| Rilzabrutinib 400 mg TID | 0.0266 | −59.25 (13.33) | −22.03 (−57.92, 13.85) | 0.2288 |
| (N = 35) | | | | |
| Rilzabrutinib 400 mg BID | 0.2138 | −46.69 (13.31) | −9.47 (−44.02, 25.08) | 0.5912 |
| (N = 35) | | | | |
| Rilzabrutinib 400 mg QPM | 0.6656 | −36.33 (12.38) | 0.88 (−32.86, 34.63) | 0.9590 |
| (N = 37) | | | | |
| Placebo (N = 36) | | −37.22 (13.40) | | |
| Week 3 | | | | |
| Rilzabrutinib 400 mg TID | 0.0449 | −72.78 (16.93) | −25.10 (−70.77, 20.57) | 0.2814 |
| (N = 35) | | | | |
| Rilzabrutinib 400 mg BID | 0.0553 | −52.68 (16.72) | −5.01 (−48.85, 38.83) | 0.8229 |
| (N = 35) | | | | |
| Rilzabrutinib 400 mg QPM | 0.8306 | −33.23 (15.73) | 14.44 (−28.58, 57.46) | 0.5105 |
| (N = 37) | | | | |
| Placebo (N = 36) | | −47.68 (17.09) | | |
| Week 4 | | | | |
| Rilzabrutinib 400 mg TID | 0.0023 | −79.54 (14.89) | −49.61 (−90.06, −9.16) | 0.0162 |
| (N = 35) | | | | |
| Rilzabrutinib 400 mg BID | 0.0123 | −58.12 (14.75) | −28.19 (−67.14, 10.77) | 0.1561 |
| (N = 35) | | | | |
| Rilzabrutinib 400 mg QPM | 0.3674 | −49.19 (13.84) | −19.26 (−57.48, 18.97) | 0.3234 |
| (N = 37) | | | | |
| Placebo (N = 36) | | −29.93 (15.25) | | |
| Week 5 | | | | |
| Rilzabrutinib 400 mg TID | 0.1474 | −72.25 (10.51) | −17.62 (−46.15, 10.90) | 0.2259 |
| (N = 35) | | | | |
| Rilzabrutinib 400 mg BID | 0.4358 | −65.60 (10.49) | −10.97 (−38.45, 16.50) | 0.4339 |
| (N = 35) | | | | |
| Rilzabrutinib 400 mg QPM | 0.7221 | −55.66 (9.83) | −1.04 (−28.01, 25.93) | 0.9399 |
| (N = 37) | | | | |
| Placebo (N = 36) | | −54.63 (10.73) | | |
| Week 6 | | | | |
| Rilzabrutinib 400 mg TID | 0.1154 | −64.16 (11.13) | −15.46 (−46.06, 15.15) | 0.3222 |
| (N = 35) | | | | |
| Rilzabrutinib 400 mg BID | 0.1753 | −69.32 (10.99) | −20.62 (−49.95, 8.72) | 0.1683 |
| (N = 35) | | | | |
| Rilzabrutinib 400 mg QPM | 0.7699 | −46.08 (10.42) | 2.62 (−26.14, 31.38) | 0.8582 |
| (N = 37) | | | | |
| Placebo (N = 36) | | −48.70 (11.62) | | |
| Week 7 | | | | |
| Rilzabrutinib 400 mg TID | 0.1636 | −67.11 (12.58) | −20.11 (−53.85, 13.63) | 0.2427 |
| (N = 35) | | | | |
| Rilzabrutinib 400 mg BID | 0.1698 | −70.13 (12.07) | −23.13 (−55.14, 8.88) | 0.1567 |
| (N = 35) | | | | |
| Rilzabrutinib 400 mg QPM | 0.2720 | −27.39 (11.77) | 19.61 (−12.44, 51.65) | 0.2304 |
| (N = 37) | | | | |
| Placebo (N = 36) | | −47.00 (12.61) | | |
| Week 8 | | | | |
| Rilzabrutinib 400 mg TID | 0.0888 | −73.05 (12.72) | −23.05 (−57.86, 11.76) | 0.1943 |
| (N = 35) | | | | |
| Rilzabrutinib 400 mg BID | 0.0281 | −80.38 (12.36) | −30.39 (−63.25, 2.47) | 0.0699 |
| (N = 35) | | | | |
| Rilzabrutinib 400 mg QPM | 0.7587 | −54.13 (11.75) | −4.14 (−36.36, 28.09) | 0.8014 |
| (N = 37) | | | | |
| Placebo (N = 36) | | −50.00 (12.91) | | |
| Week 9 | | | | |
| Rilzabrutinib 400 mg TID | 0.0127 | −84.73 (12.57) | −37.56 (−71.28, −3.83) | 0.0291 |
| (N = 35) | | | | |
| Rilzabrutinib 400 mg BID | 0.0342 | −74.80 (12.27) | −27.62 (−59.82, 4.58) | 0.0927 |
| (N = 35) | | | | |
| Rilzabrutinib 400 mg QPM | 0.7262 | −47.25 (11.58) | −0.07 (−31.59, 31.45) | 0.9964 |
| (N = 37) | | | | |
| Placebo (N = 36) | | −47.18 (12.47) | | |
| Week 10 | | | | |
| Rilzabrutinib 400 mg TID | 0.0088 | −89.99 (16.85) | −46.10 (−91.54, −0.67) | 0.0467 |
| (N = 35) | | | | |
| Rilzabrutinib 400 mg BID | 0.0953 | −60.11 (16.65) | −16.22 (−60.13, 27.69) | 0.4690 |
| (N = 35) | | | | |
| Rilzabrutinib 400 mg QPM | 0.8887 | −48.11 (15.70) | −4.23 (−47.40, 38.95) | 0.8478 |
| (N = 37) | | | | |
| Placebo (N = 36) | | −43.89 (17.02) | | |
| Week 11 | | | | |
| Rilzabrutinib 400 mg TID | 0.1414 | −78.48 (11.38) | −20.16 (−50.91, 10.58) | 0.1986 |
| (N = 35) | | | | |
| Rilzabrutinib 400 mg BID | 0.1255 | −83.14 (11.48) | −24.83 (−55.17, 5.52) | 0.1088 |
| (N = 35) | | | | |
| Rilzabrutinib 400 mg QPM | 0.4749 | −52.05 (10.68) | 6.27 (−23.29, 35.83) | 0.6775 |
| (N = 37) | | | | |
| Placebo (N = 36) | | −58.32 (11.64) | | |
| Week 12 | | | | |
| Rilzabrutinib 400 mg TID | 0.0797 | −84.63 (10.83) | −23.26 (−52.37, 5.85) | 0.1173 |
| (N = 35) | | | | |
| Rilzabrutinib 400 mg BID | 0.1685 | −86.30 (10.67) | −24.94 (−53.17, 3.29) | 0.0834 |
| (N = 35) | | | | |
| Rilzabrutinib 400 mg QPM | 0.2708 | −52.66 (10.25) | 8.71 (−19.46, 36.88) | 0.5444 |
| (N = 37) | | | | |
| Placebo (N = 36) | | −61.36 (10.86) | | |

^{a}Each of the imputed complete data were analyzed by fitting an ANCOVA model with the corresponding baseline value, intervention group and regions as covariates.

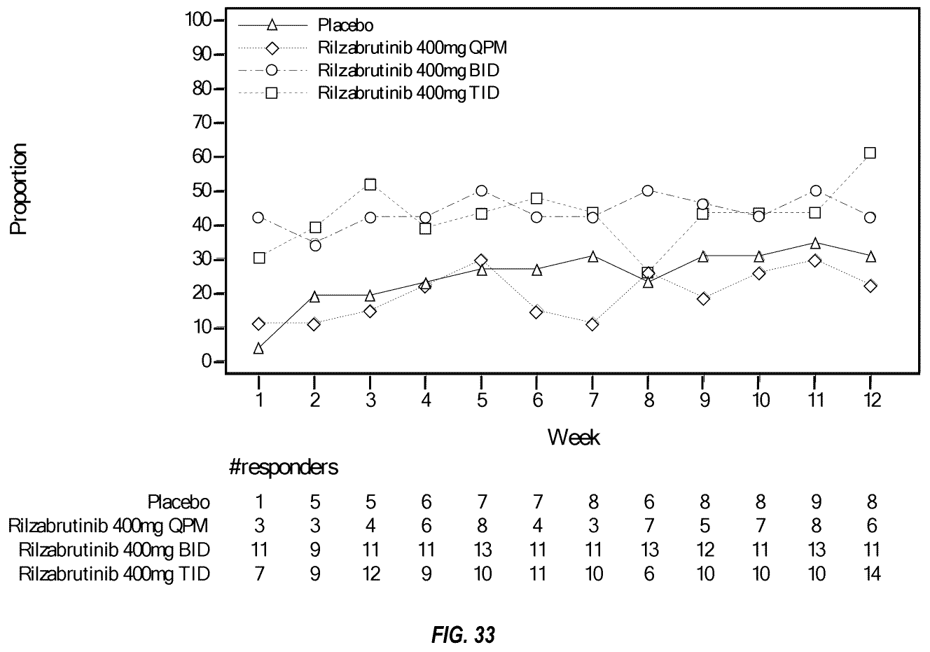

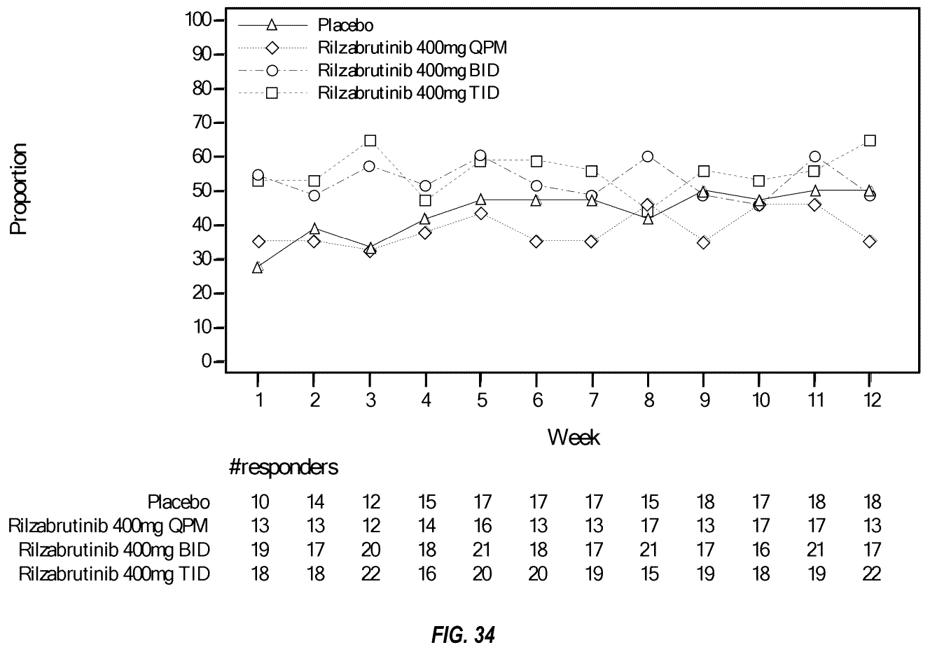

A substantial percentage of patients receiving 400 mg BID or TID rilzabrutinib achieved angioedema scores of 0 (see FIGS. 33-34 and Tables 43-44. Notably, in the omalizumab-naïve population, 42.3% of patients in the 400 mg BID arm and 30.4% of patients in the 400 mg TID arm achieved scores of 0 within one week of initiating treatment. Further, at Week 12, 42.3% of omalizumab-naïve patients in the 400 mg BID arm and 60.9% of omalizumab-naïve patients in the 400 mg TID arm had scores of 0, indicating that the improvements were sustained over the course of the study.

TABLE 43
Proportion of Participants with AAS7 = 0 over Time up to Week 12 (Participants
with Angioedema at Baseline in Omalizumab-Naïve Population)

| | | | | RRD (%) | | |
|---|---|---|---|---|---|---|
| | | | Imputed | for | OR (95% | |
| | | Non- | non- | rilzabrutinib | CI) for | |
| | Responder | responder | responder | vs. placebo | rilzabrutinib | P-value vs. |
| | n (%) | n (%) | n (%) | (95% CI)^{a} | vs. placebo^{a} | placebo^{b} |
| Baseline | | | | | | |
| Rilzabrutinib 400 mg TID (N = 23) | 0 | 23 (100) | 0 | | | |
| Rilzabrutinib 400 mg BID (N = 26) | 0 | 26 (100) | 0 | | | |
| Rilzabrutinib 400 mg QPM (N = 27) | 0 | 27 (100) | 0 | | | |
| Placebo (N = 26) | 0 | 26 (100) | 0 | | | |
| Week 1 | | | | | | |
| Rilzabrutinib 400 mg TID (N = 23) | 7 (30.4) | 16 (69.6) | 0 | 21.8 (1.41, | 9.250 (0.901, | 0.0399 |
| | | | | 42.21) | 94.977) | |
| Rilzabrutinib 400 mg BID (N = 26) | 11 (42.3) | 15 (57.7) | 0 | 35.1 (14.62, | 20.850 (2.026, | 0.0019 |
| | | | | 55.52) | 214.582) | |
| Rilzabrutinib 400 mg QPM (N = 27) | 3 (11.1) | 24 (88.9) | 0 | 9.1 (−5.96, | 3.545 (0.387, | 0.2210 |
| | | | | 24.23) | 32.487) | |
| Placebo (N = 26) | 1 (3.8) | 25 (96.2) | 0 | | | |
| Week 2 | | | | | | |
| Rilzabrutinib 400 mg TID (N = 23) | 9 (39.1) | 14 (60.9) | 0 | 12.9 (−13.45, | 1.922 (0.506, | 0.3449 |
| | | | | 39.17) | 7.307) | |
| Rilzabrutinib 400 mg BID (N = 26) | 9 (34.6) | 17 (65.4) | 1 (3.8) | 14.6 (−10.55, | 2.012 (0.587, | 0.2526 |
| | | | | 39.70) | 6.895) | |
| Rilzabrutinib 400 mg QPM (N = 27) | 3 (11.1) | 24 (88.9) | 0 | −9.2 (−29.54, | 0.514 (0.114, | 0.3750 |
| | | | | 11.20) | 2.332) | |
| Placebo (N = 26) | 5 (19.2) | 21 (80.8) | 1 (3.8) | | | |
| Week 3 | | | | | | |
| Rilzabrutinib 400 mg TID (N = 23) | 12 (52.2) | 11 (47.8) | 0 | 22.5 (−4.30, | 3.063 (0.815, | 0.0959 |
| | | | | 49.32) | 11.508) | |
| Rilzabrutinib 400 mg BID (N = 26) | 11 (42.3) | 15 (57.7) | 0 | 20.8 (−4.36, | 2.703 (0.776, | 0.1107 |
| | | | | 45.88) | 9.417) | |
| Rilzabrutinib 400 mg QPM (N = 27) | 4 (14.8) | 23 (85.2) | 0 | −5.0 (−25.05, | 0.686 (0.153, | 0.6344 |
| | | | | 14.97) | 3.070) | |
| Placebo (N = 26) | 5 (19.2) | 21 (80.8) | 1 (3.8) | | | |
| Week 4 | | | | | | |
| Rilzabrutinib 400 mg TID (N = 23) | 9 (39.1) | 14 (60.9) | 0 | 7.3 (−17.51, | 1.519 (0.372, | 0.5701 |
| | | | | 32.12) | 6.200) | |
| Rilzabrutinib 400 mg BID (N = 26) | 11 (42.3) | 15 (57.7) | 1 (3.8) | 16.8 (−9.25, | 2.118 (0.633, | 0.2174 |
| | | | | 42.82) | 7.081) | |
| Rilzabrutinib 400 mg QPM (N = 27) | 6 (22.2) | 21 (77.8) | 1 (3.7) | −2.4 (−24.90, | 0.869 (0.230, | 0.8394 |
| | | | | 20.12) | 3.275) | |
| Placebo (N = 26) | 6 (23.1) | 20 (76.9) | 2 (7.7) | | | |
| Week 5 | | | | | | |
| Rilzabrutinib 400 mg TID (N = 23) | 10 (43.5) | 13 (56.5) | 0 | 9.8 (−18.19, | 1.550 (0.448, | 0.4959 |
| | | | | 37.86) | 5.366) | |
| Rilzabrutinib 400 mg BID (N = 26) | 13 (50.0) | 13 (50.0) | 2 (7.7) | 22.3 (−5.17, | 2.390 (0.769, | 0.1186 |
| | | | | 49.87) | 7.427) | |
| Rilzabrutinib 400 mg QPM (N = 27) | 8 (29.6) | 19 (70.4) | 2 (7.4) | 1.6 (−23.44, | 1.079 (0.327, | 0.9006 |
| | | | | 26.63) | 3.558) | |
| Placebo (N = 26) | 7 (26.9) | 19 (73.1) | 3 (11.5) | | | |
| Week 6 | | | | | | |
| Rilzabrutinib 400 mg TID (N = 23) | 11 (47.8) | 12 (52.2) | 2 (8.7) | 13.5 (−13.95, | 1.874 (0.528, | 0.3486 |
| | | | | 41.01) | 6.650) | |
| Rilzabrutinib 400 mg BID (N = 26) | 11 (42.3) | 15 (57.7) | 2 (7.7) | 13.6 (−12.56, | 1.828 (0.565, | 0.3156 |
| | | | | 39.77) | 5.910) | |
| Rilzabrutinib 400 mg QPM (N = 27) | 4 (14.8) | 23 (85.2) | 4 (14.8) | −13.0 (−34.72, | 0.437 (0.106, | 0.2620 |
| | | | | 8.69) | 1.796) | |
| Placebo (N = 26) | 7 (26.9) | 19 (73.1) | 5 (19.2) | | | |
| Week 7 | | | | | | |
| Rilzabrutinib 400 mg TID (N = 23) | 10 (43.5) | 13 (56.5) | 3 (13.0) | 7.2 (−21.57, | 1.351 (0.407, | 0.6220 |
| | | | | 35.92) | 4.489) | |
| Rilzabrutinib 400 mg BID (N = 26) | 11 (42.3) | 15 (57.7) | 2 (7.7) | 10.5 (−17.21, | 1.515 (0.499, | 0.4586 |
| | | | | 38.23) | 4.599) | |
| Rilzabrutinib 400 mg QPM (N = 27) | 3 (11.1) | 24 (88.9) | 6 (22.2) | −23.0 (−43.69, | 0.200 (0.039, | 0.0441 |
| | | | | −2.37) | 1.034) | |
| Placebo (N = 26) | 8 (30.8) | 18 (69.2) | 4 (15.4) | | | |
| Week 8 | | | | | | |
| Rilzabrutinib 400 mg TID (N = 23) | 6 (26.1) | 17 (73.9) | 4 (17.4) | −6.2 (−29.94, | 0.690 (0.160, | 0.6203 |
| | | | | 17.56) | 2.969) | |
| Rilzabrutinib 400 mg BID (N = 26) | 13 (50.0) | 13 (50.0) | 3 (11.5) | 26.4 (−0.77, | 2.829 (0.886, | 0.0626 |
| | | | | 53.60) | 9.031) | |
| Rilzabrutinib 400 mg QPM (N = 27) | 7 (25.9) | 20 (74.1) | 7 (25.9) | −1.3 (−24.15, | 0.929 (0.251, | 0.9119 |
| | | | | 21.56) | 3.430) | |
| Placebo (N = 26) | 6 (23.1) | 20 (76.9) | 4 (15.4) | | | |
| Week 9 | | | | | | |
| Rilzabrutinib 400 mg TID (N = 23) | 10 (43.5) | 13 (56.5) | 3 (13.0) | 6.9 (−21.07, | 1.358 (0.394, | 0.6178 |
| | | | | 34.92) | 4.681) | |
| Rilzabrutinib 400 mg BID (N = 26) | 12 (46.2) | 14 (53.8) | 4 (15.4) | 14.5 (−12.95, | 1.787 (0.578, | 0.3088 |
| | | | | 41.93) | 5.519) | |
| Rilzabrutinib 400 mg QPM (N = 27) | 5 (18.5) | 22 (81.5) | 6 (22.2) | −15.5 (−38.66, | 0.431 (0.116, | 0.2062 |
| | | | | 7.74) | 1.602) | |
| Placebo (N = 26) | 8 (30.8) | 18 (69.2) | 5 (19.2) | | | |
| Week 10 | | | | | | |
| Rilzabrutinib 400 mg TID (N = 23) | 10 (43.5) | 13 (56.5) | 2 (8.7) | 11.0 (−16.84, | 1.627 (0.465, | 0.4184 |
| | | | | 38.85) | 5.690) | |
| Rilzabrutinib 400 mg BID (N = 26) | 11 (42.3) | 15 (57.7) | 6 (23.1) | 13.1 (−13.64, | 1.734 (0.544, | 0.3524 |
| | | | | 39.79) | 5.526) | |
| Rilzabrutinib 400 mg QPM (N = 27) | 7 (25.9) | 20 (74.1) | 6 (22.2) | −9.2 (−32.92, | 0.616 (0.174, | 0.4559 |
| | | | | 14.48) | 2.186) | |
| Placebo (N = 26) | 8 (30.8) | 18 (69.2) | 4 (15.4) | | | |
| Week 11 | | | | | | |
| Rilzabrutinib 400 mg TID (N = 23) | 10 (43.5) | 13 (56.5) | 2 (8.7) | 3.1 (−23.73, | 1.160 (0.319, | 0.8269 |
| | | | | 29.88) | 4.214) | |
| Rilzabrutinib 400 mg BID (N = 26) | 13 (50.0) | 13 (50.0) | 6 (23.1) | 17.8 (−9.70, | 2.010 (0.651, | 0.2207 |
| | | | | 45.21) | 6.204) | |
| Rilzabrutinib 400 mg QPM (N = 27) | 8 (29.6) | 19 (70.4) | 6 (22.2) | −8.6 (−32.83, | 0.650 (0.189, | 0.5103 |
| | | | | 15.69) | 2.239) | |
| Placebo (N = 26) | 9 (34.6) | 17 (65.4) | 4 (15.4) | | | |
| Week 12 | | | | | | |
| Rilzabrutinib 400 mg TID (N = 23) | 14 (60.9) | 9 (39.1) | 4 (17.4) | 29.7 (0.65, | 3.085 (0.945, | 0.0526 |
| | | | | 58.69) | 10.074) | |
| Rilzabrutinib 400 mg BID (N = 26) | 11 (42.3) | 15 (57.7) | 8 (30.8) | 11.3 (−15.22, | 1.627 (0.513, | 0.4132 |
| | | | | 37.83) | 5.167) | |
| Rilzabrutinib 400 mg QPM (N = 27) | 6 (22.2) | 21 (77.8) | 9 (33.3) | −11.2 (−35.56, | 0.571 (0.165, | 0.3734 |
| | | | | 13.12) | 1.976) | |
| Placebo (N = 26) | 8 (30.8) | 18 (69.2) | 5 (19.2) | | | |

CMH: Cochran-Mantel Haenzel
^{a}OR: odds ratio; RRD: response rate difference; derived from the Mantel-Haenszel estimator

TABLE 44
Proportion of Participants with AAS7 = 0 over Time up to Week 12
(Omalizumab-Naïve Population with Outlier Removed from Analysis)

| | | | | RRD (%) | | |
|---|---|---|---|---|---|---|
| | | | Imputed | for | OR (95% | |
| | | Non- | non- | rilzabrutinib | CI) for | |
| | Responder | responder | responder | vs. placebo | rilzabrutinib | P-value vs. |
| | n (%) | n (%) | n (%) | (95% CI)^{a} | vs. placebo^{a} | placebo^{b} |
| Baseline | | | | | | |
| Rilzabrutinib 400 mg TID (N = 34) | 11 (32.4) | 23 (67.6) | 0 | | | |
| Rilzabrutinib 400 mg BID (N = 35) | 9 (25.7) | 26 (74.3) | 0 | | | |
| Rilzabrutinib 400 mg QPM (N = 37) | 10 (27.0) | 27 (73.0) | 0 | | | |
| Placebo (N = 36) | 10 (27.8) | 26 (72.2) | 0 | | | |
| Week 1 | | | | | | |
| Rilzabrutinib 400 mg TID (N = 34) | 18 (52.9) | 16 (47.1) | 0 | 21.8 (−0.83, | 2.576 (0.940, | 0.0644 |
| | | | | 44.41) | 7.060) | |
| Rilzabrutinib 400 mg BID (N = 35) | 19 (54.3) | 16 (45.7) | 0 | 23.4 (1.75, | 2.944 (1.046, | 0.0408 |
| | | | | 45.01) | 8.292) | |
| Rilzabrutinib 400 mg QPM (N = 37) | 13 (35.1) | 24 (64.9) | 0 | 8.1 (−13.79, | 1.428 (0.543, | 0.4649 |
| | | | | 30.07) | 3.758) | |
| Placebo (N = 36) | 10 (27.8) | 26 (72.2) | 0 | | | |
| Week 2 | | | | | | |
| Rilzabrutinib 400 mg TID (N = 34) | 18 (52.9) | 16 (47.1) | 0 | 10.2 (−12.61, | 1.556 (0.579, | 0.3883 |
| | | | | 33.08) | 4.185) | |
| Rilzabrutinib 400 mg BID (N = 35) | 17 (48.6) | 18 (51.4) | 1 (2.9) | 8.2 (−15.79, | 1.366 (0.549, | 0.4941 |
| | | | | 32.29) | 3.395) | |
| Rilzabrutinib 400 mg QPM (N = 37) | 13 (35.1) | 24 (64.9) | 0 | −3.3 (−26.15, | 0.875 (0.347, | 0.7761 |
| | | | | 19.57) | 2.209) | |
| Placebo (N = 36) | 14 (38.9) | 22 (61.1) | 1 (2.8) | | | |
| Week 3 | | | | | | |
| Rilzabrutinib 400 mg TID (N = 34) | 22 (64.7) | 12 (35.3) | 0 | 26.5 (3.50, | 3.010 (1.126, | 0.0228 |
| | | | | 49.54) | 8.044) | |
| Rilzabrutinib 400 mg BID (N = 35) | 20 (57.1) | 15 (42.9) | 0 | 21.6 (−1.62, | 2.357 (0.913, | 0.0679 |
| | | | | 44.84) | 6.082) | |
| Rilzabrutinib 400 mg QPM (N = 37) | 12 (32.4) | 25 (67.6) | 0 | 0.9 (−20.42, | 1.041 (0.385, | 0.9362 |
| | | | | 22.15) | 2.817) | |
| Placebo (N = 36) | 12 (33.3) | 24 (66.7) | 1 (2.8) | | | |
| Week 4 | | | | | | |
| Rilzabrutinib 400 mg TID (N = 34) | 16 (47.1) | 18 (52.9) | 2 (5.9) | 1.1 (−21.36, | 1.049 (0.384, | 0.9267 |
| | | | | 23.50) | 2.863) | |
| Rilzabrutinib 400 mg BID (N = 35) | 18 (51.4) | 17 (48.6) | 2 (5.7) | 7.4 (−15.53, | 1.361 (0.523, | 0.5317 |
| | | | | 30.33) | 3.539) | |
| Rilzabrutinib 400 mg QPM (N = 37) | 14 (37.8) | 23 (62.2) | 1 (2.7) | −3.0 (−24.84, | 0.874 (0.331, | 0.7907 |
| | | | | 18.78) | 2.307) | |
| Placebo (N = 36) | 15 (41.7) | 21 (58.3) | 2 (5.6) | | | |
| Week 5 | | | | | | |
| Rilzabrutinib 400 mg TID (N = 34) | 20 (58.8) | 14 (41.2) | 1 (2.9) | 7.5 (−15.98, | 1.359 (0.520, | 0.5302 |
| | | | | 30.99) | 3.550) | |
| Rilzabrutinib 400 mg BID (N = 35) | 21 (60.0) | 14 (40.0) | 2 (5.7) | 11.1 (−11.98, | 1.574 (0.610, | 0.3477 |
| | | | | 34.23) | 4.061) | |
| Rilzabrutinib 400 mg QPM (N = 37) | 16 (43.2) | 21 (56.8) | 2 (5.4) | −3.4 (−26.11, | 0.872 (0.344, | 0.7714 |
| | | | | 19.41) | 2.211) | |
| Placebo (N = 36) | 17 (47.2) | 19 (52.8) | 3 (8.3) | | | |
| Week 6 | | | | | | |
| Rilzabrutinib 400 mg TID (N = 34) | 20 (58.8) | 14 (41.2) | 3 (8.8) | 6.5 (−16.05, | 1.334 (0.490, | 0.5811 |
| | | | | 28.96) | 3.635) | |
| Rilzabrutinib 400 mg BID (N = 35) | 18 (51.4) | 17 (48.6) | 2 (5.7) | 2.9 (−20.98, | 1.118 (0.447, | 0.8106 |
| | | | | 26.81) | 2.795) | |
| Rilzabrutinib 400 mg QPM (N = 37) | 13 (35.1) | 24 (64.9) | 4 (10.8) | −11.4 (−33.39, | 0.606 (0.231, | 0.3192 |
| | | | | 10.50) | 1.594) | |
| Placebo (N = 36) | 17 (47.2) | 19 (52.8) | 5 (13.9) | | | |
| Week 7 | | | | | | |
| Rilzabrutinib 400 mg TID (N = 34) | 19 (55.9) | 15 (44.1) | 4 (11.8) | 6.5 (−17.94, | 1.277 (0.509, | 0.5961 |
| | | | | 30.95) | 3.205) | |
| Rilzabrutinib 400 mg BID (N = 35) | 17 (48.6) | 18 (51.4) | 2 (5.7) | 0.6 (−23.63, | 1.022 (0.414, | 0.9620 |
| | | | | 24.78) | 2.525) | |
| Rilzabrutinib 400 mg QPM (N = 37) | 13 (35.1) | 24 (64.9) | 6 (16.2) | −11.8 (−34.27, | 0.611 (0.238, | 0.3105 |
| | | | | 10.63) | 1.570) | |
| Placebo (N = 36) | 17 (47.2) | 19 (52.8) | 4 (11.1) | | | |
| Week 8 | | | | | | |
| Rilzabrutinib 400 mg TID (N = 34) | 15 (44.1) | 19 (55.9) | 6 (17.6) | −0.9 (−24.66, | 0.964 (0.373, | 0.9387 |
| | | | | 22.82) | 2.489) | |
| Rilzabrutinib 400 mg BID (N = 35) | 21 (60.0) | 14 (40.0) | 3 (8.6) | 17.0 (−6.09, | 1.991 (0.768, | 0.1565 |
| | | | | 40.01) | 5.157) | |
| Rilzabrutinib 400 mg QPM (N = 37) | 17 (45.9) | 20 (54.1) | 7 (18.9) | 4.2 (−18.73, | 1.185 (0.471, | 0.7177 |
| | | | | 27.17) | 2.982) | |
| Placebo (N = 36) | 15 (41.7) | 21 (58.3) | 4 (11.1) | | | |
| Week 9 | | | | | | |
| Rilzabrutinib 400 mg TID (N = 34) | 19 (55.9) | 15 (44.1) | 5 (14.7) | 3.6 (−20.13, | 1.155 (0.448, | 0.7579 |
| | | | | 27.35) | 2.981) | |
| Rilzabrutinib 400 mg BID (N = 35) | 17 (48.6) | 18 (51.4) | 7 (20.0) | −2.1 (−25.67, | 0.919 (0.363, | 0.8577 |
| | | | | 21.40) | 2.330) | |
| Rilzabrutinib 400 mg QPM (N = 37) | 13 (35.1) | 24 (64.9) | 6 (16.2) | −15.0 (−36.92, | 0.520 (0.197, | 0.1891 |
| | | | | 6.90) | 1.373) | |
| Placebo (N = 36) | 18 (50.0) | 18 (50.0) | 5 (13.9) | | | |
| Week 10 | | | | | | |
| Rilzabrutinib 400 mg TID (N = 34) | 18 (52.9) | 16 (47.1) | 4 (11.8) | 6.0 (−18.63, | 1.247 (0.499, | 0.6201 |
| | | | | 30.54) | 3.119) | |
| Rilzabrutinib 400 mg BID (N = 35) | 16 (45.7) | 19 (54.3) | 8 (22.9) | −1.4 (−24.86, | 0.945 (0.371, | 0.9055 |
| | | | | 22.01) | 2.404) | |
| Rilzabrutinib 400 mg QPM (N = 37) | 17 (45.9) | 20 (54.1) | 6 (16.2) | −1.9 (−25.10, | 0.927 (0.372, | 0.8717 |
| | | | | 21.27) | 2.311) | |
| Placebo (N = 36) | 17 (47.2) | 19 (52.8) | 4 (11.1) | | | |
| Week 11 | | | | | | |
| Rilzabrutinib 400 mg TID (N = 34) | 19 (55.9) | 15 (44.1) | 3 (8.8) | 3.1 (−19.84, | 1.142 (0.427, | 0.7922 |
| | | | | 26.01) | 3.049) | |
| Rilzabrutinib 400 mg BID (N = 35) | 21 (60.0) | 14 (40.0) | 7 (20.0) | 10.4 (−12.89, | 1.516 (0.592, | 0.3828 |
| | | | | 33.65) | 3.884) | |
| Rilzabrutinib 400 mg QPM (N = 37) | 17 (45.9) | 20 (54.1) | 7 (18.9) | −4.7 (−26.95, | 0.819 (0.316, | 0.6860 |
| | | | | 17.57) | 2.121) | |
| Placebo (N = 36) | 18 (50.0) | 18 (50.0) | 4 (11.1) | | | |
| Week 12 | | | | | | |
| Rilzabrutinib 400 mg TID (N = 34) | 22 (64.7) | 12 (35.3) | 7 (20.6) | 13.0 (−10.57, | 1.685 (0.646, | 0.2803 |
| | | | | 36.55) | 4.396) | |
| Rilzabrutinib 400 mg BID (N = 35) | 17 (48.6) | 18 (51.4) | 11 (31.4) | −1.9 (−25.26, | 0.925 (0.362, | 0.8730 |
| | | | | 21.39) | 2.365) | |
| Rilzabrutinib 400 mg QPM (N = 37) | 13 (35.1) | 24 (64.9) | 10 (27.0) | −15.0 (−36.78, | 0.516 (0.194, | 0.1891 |
| | | | | 6.76) | 1.370) | |
| Placebo (N = 36) | 18 (50.0) | 18 (50.0) | 5 (13.9) | | | |

^{a}OR: odds ratio; RRD: response rate difference; derived from the Mantel-Haenszel estimator

### UCT

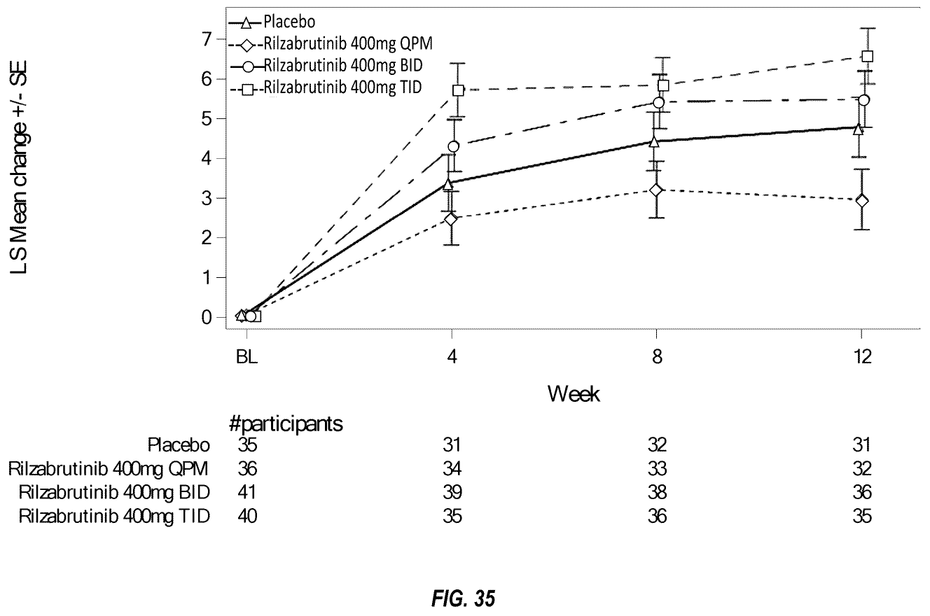

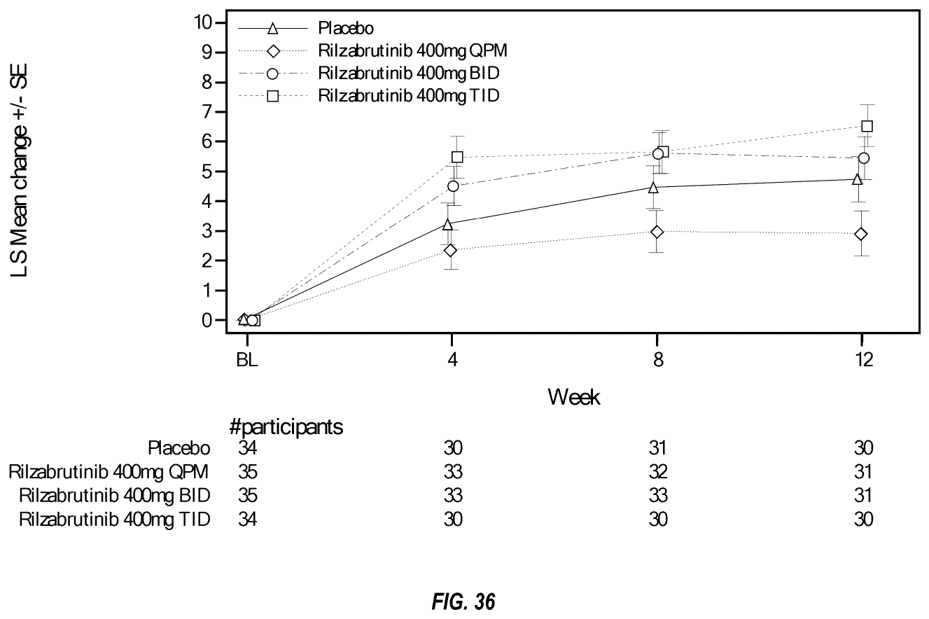

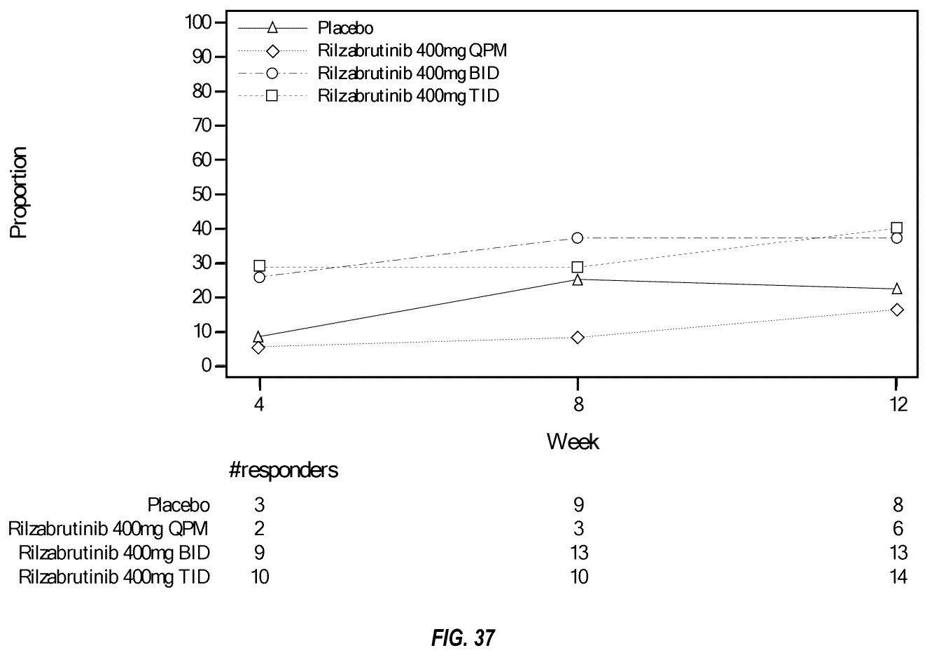

Overall, patients in the 400 mg TID cohort experienced a mild increase in UCT over baseline, with >20% of patients achieving a UCT≥12 over time up to Week 12 (see FIGS. 35-37 and Tables 45-47).

TABLE 45
Summary of UCT by Visit in the Double-Blind Period

| | Omalizumab-Naïve Population | | | | | | | |
|---|---|---|---|---|---|---|---|---|
| | | | Rilzabrutinib | | Rilzabrutinib | | Rilzabrutinib | |
| | Placebo | | 400 mg QPM | | 400 mg BID | | 400 mg TID | |
| UCT | (N = 36) | | (N = 37) | | (N = 35) | | (N = 35) | |
| Baseline | | | | | | | | |
| Number | 34 | | 35 | | 35 | | 34 | |
| Mean (SD) | 4.29 | (3.08) | 4.00 | (2.18) | 4.51 | (3.21) | 4.47 | (3.54) |
| Median | 4.00 | | 4.00 | | 4.00 | | 4.00 | |
| Q1; Q3 | 2.00; 6.00 | | 2.00; 6.00 | | 2.00; 7.00 | | 2.00; 6.00 | |
| Min; Max | 0.0; 12.0 | | 0.0; 9.0 | | 0.0; 13.0 | | 0.0; 15.0 | |
| Week 4 | | | | | | | | |
| Number (observed/imputed) | 30 | (30/0) | 34 | (33/1) | 33 | (33/0) | 30 | (30/0) |
| Mean (SD) | 7.47 | (4.07) | 6.35 | (3.14) | 8.85 | (4.53) | 9.77 | (4.17) |
| Median | 8.00 | | 6.00 | | 9.00 | | 10.50 | |
| Q1; Q3 | 4.00; 11.00 | | 4.00; 8.00 | | 6.00; 12.00 | | 7.00; 12.00 | |
| Min; Max | 0.0; 16.0 | | 1.0; 14.0 | | 1.0; 16.0 | | 1.0; 16.0 | |
| Change from baseline | | | | | | | | |
| Number (observed/imputed) | 30 | (30/0) | 33 | (32/1) | 33 | (33/0) | 30 | (30/0) |
| Mean (SD) | 2.80 | (3.51) | 2.27 | (3.07) | 4.21 | (4.97) | 5.63 | (4.70) |
| Median | 3.00 | | 2.00 | | 4.00 | | 6.50 | |
| Q1; Q3 | 1.00; 6.00 | | 0.00; 5.00 | | 1.00; 7.00 | | 2.00; 9.00 | |
| Min; Max | −6.0; 9.0 | | −3.0; 8.0 | | −6.0; 15.0 | | −3.0; 15.0 | |
| Percent change from baseline | | | | | | | | |
| Number (observed/imputed) | 27 | (27/0) | 32 | (31/1) | 31 | (31/0) | 26 | (26/0) |
| Mean (SD) | 76.17 | (117.81) | 102.47 | (169.77) | 220.19 | (390.12) | 254.30 | (370.78) |
| Median | 50.00 | | 45.00 | | 75.00 | | 100.00 | |
| Q1; Q3 | 0.00; 120.00 | | 0.00; 135.00 | | 20.00; 166.67 | | 33.33; 275.00 | |
| Min; Max | −100.0; 450.0 | | −42.9; 600.0 | | −80.0; 1500.0 | | −37.5; 1500.0 | |
| Week 8 | | | | | | | | |
| Number (observed/imputed) | 31 | (30/1) | 32 | (29/3) | 33 | (32/1) | 30 | (30/0) |
| Mean (SD) | 8.65 | (4.36) | 6.84 | (3.84) | 10.12 | (4.20) | 9.80 | (3.86) |
| Median | 10.00 | | 7.00 | | 11.00 | | 9.50 | |
| Q1; Q3 | 5.00; 12.00 | | 4.00; 10.00 | | 7.00; 14.00 | | 8.00; 13.00 | |
| Min; Max | 0.0; 16.0 | | 0.0; 16.0 | | 1.0; 16.0 | | 2.0; 16.0 | |
| Change from baseline | | | | | | | | |
| Number (observed/imputed) | 31 | (30/1) | 32 | (29/3) | 33 | (32/1) | 30 | (30/0) |
| Mean (SD) | 4.16 | (3.60) | 3.09 | (3.84) | 5.70 | (5.00) | 6.20 | (3.99) |
| Median | 3.00 | | 2.00 | | 5.00 | | 6.50 | |
| Q1; Q3 | 1.00; 8.00 | | 0.00; 6.00 | | 2.00; 9.00 | | 3.00; 9.00 | |
| Min; Max | −1.0; 11.0 | | −3.0; 11.0 | | −6.0; 15.0 | | −3.0; 14.0 | |
| Percent change from baseline | | | | | | | | |
| Number (observed/imputed) | 27 | (26/1) | 31 | (28/3) | 30 | (29/1) | 26 | (26/0) |
| Mean (SD) | 100.69 | (122.18) | 138.85 | (261.15) | 290.62 | (401.91) | 277.48 | (345.91) |
| Median | 62.50 | | 66.67 | | 114.29 | | 120.83 | |
| Q1; Q3 | 33.33; 133.33 | | 0.00; 150.00 | | 40.00; 400.00 | | 75.00; 300.00 | |
| Min; Max | −100.0; 400.0 | | −100.0; 1000.0 | | −46.2; 1500.0 | | −50.0; 1200.0 | |
| Week 12 | | | | | | | | |
| Number (observed/imputed) | 30 | (29/1) | 33 | (30/3) | 31 | (29/2) | 30 | (29/1) |
| Mean (SD) | 8.73 | (4.23) | 6.88 | (3.86) | 10.03 | (4.29) | 10.50 | (4.23) |
| Median | 9.00 | | 7.00 | | 11.00 | | 11.00 | |
| Q1; Q3 | 6.00; 12.00 | | 4.00; 10.00 | | 7.00; 12.00 | | 8.00; 13.00 | |
| Min; Max | 0.0; 16.0 | | 0.0; 13.0 | | 0.0; 16.0 | | 1.0; 16.0 | |
| Change from baseline | | | | | | | | |
| Number (observed/imputed) | 30 | (29/1) | 31 | (28/3) | 31 | (29/2) | 30 | (29/1) |
| Mean (SD) | 4.43 | (3.48) | 3.13 | (3.69) | 5.65 | (5.32) | 6.67 | (4.92) |
| Median | 4.00 | | 2.00 | | 4.00 | | 6.00 | |
| Q1; Q3 | 3.00; 7.00 | | 0.00; 6.00 | | 1.00; 11.00 | | 3.00; 11.00 | |
| Min; Max | −3.0; 11.0 | | −4.0; 11.0 | | −6.0; 14.0 | | −2.0; 16.0 | |
| Percent change from baseline | | | | | | | | |
| Number (observed/imputed) | 26 | (25/1) | 30 | (27/3) | 28 | (26/2) | 26 | (26/0) |
| Mean (SD) | 131.15 | (177.29) | 121.74 | (233.08) | 301.53 | (394.02) | 311.71 | (436.58) |
| Median | 100.00 | | 92.86 | | 130.95 | | 102.86 | |
| Q1; Q3 | 33.33; 220.00 | | 0.00; 140.00 | | 28.33; 416.67 | | 60.00; 300.00 | |
| Min; Max | −100.0; 800.0 | | −100.0; 1100.0 | | −46.2; 1400.0 | | −66.7; 1500.0 | |
| End of study | | | | | | | | |
| Number (observed/imputed) | 5 | (4/1) | 2 | (2/0) | 3 | (3/0) | 3 | (3/0) |
| Mean (SD) | 8.40 | (3.44) | 11.50 | (0.71) | 6.67 | (8.14) | 10.33 | (4.51) |
| Median | 7.00 | | 11.50 | | 3.00 | | 10.00 | |
| Q1; Q3 | 6.00; 11.00 | | 11.00; 12.00 | | 1.00; 16.00 | | 6.00; 15.00 | |
| Min; Max | 5.0; 13.0 | | 11.0; 12.0 | | 1.0; 16.0 | | 6.0; 15.0 | |
| Change from baseline | | | | | | | | |
| Number (observed/imputed) | 5 | (4/1) | 2 | (2/0) | 3 | (3/0) | 3 | (3/0) |
| Mean (SD) | 4.60 | (2.70) | 8.50 | (0.71) | 0.00 | (5.29) | 4.00 | (2.00) |
| Median | 5.00 | | 8.50 | | −2.00 | | 4.00 | |
| Q1; Q3 | 3.00; 6.00 | | 8.00; 9.00 | | −4.00; 6.00 | | 2.00; 6.00 | |
| Min; Max | 1.0; 8.0 | | 8.0; 9.0 | | −4.0; 6.0 | | 2.0; 6.0 | |
| Percent change from baseline | | | | | | | | |
| Number (observed/imputed) | 4 | (3/1) | 2 | (2/0) | 3 | (3/0) | 2 | (2/0) |
| Mean (SD) | 217.50 | (262.35) | 283.33 | (23.57) | −21.27 | (70.54) | 41.03 | (36.26) |

| | | | | |
|---|---|---|---|---|
| Median | 130.00 | 283.33 | −57.14 | 41.03 |
| Q1; Q3 | 55.00; 380.00 | 266.67; 300.00 | −66.67; 60.00 | 15.38; 66.67 |
| Min; Max | 10.0; 600.0 | 266.7; 300.0 | −66.7; 60.0 | 15.4; 66.7 |

| | Patients with Prior Omalizumab Use, | | | | | | | |
|---|---|---|---|---|---|---|---|---|
| | Including Omalizumab-Incomplete Responders | | | | | | | |
| | | | Rilzabrutinib | | Rilzabrutinib | | Rilzabrutinib | |
| | Placebo | | 400 mg QPM | | 400 mg BID | | 400 mg TID | |
| UCT | (N = 4) | | (N = 1) | | (N = 6) | | (N = 6) | |
| Baseline | | | | | | | | |
| Number | 1 | | 1 | | 6 | | 6 | |
| Mean (SD) | 0.00 | (NC) | 5.00 | (NC) | 2.67 | (1.03) | 4.83 | (3.60) |
| Median | 0.00 | | 5.00 | | 3.00 | | 4.00 | |
| Q1; Q3 | 0.00; 0.00 | | 5.00; 5.00 | | 2.00; 3.00 | | 3.00; 8.00 | |
| Min; Max | 0.0; 0.0 | | 5.0; 5.0 | | 1.0; 4.0 | | 0.0; 10.0 | |
| Week 4 | | | | | | | | |
| Number (observed/imputed) | 3 | (3/0) | 1 | (1/0) | 6 | (5/1) | 5 | (5/0) |
| Mean (SD) | 4.33 | (3.51) | 8.00 | (NC) | 7.00 | (7.18) | 11.60 | (4.51) |
| Median | 4.00 | | 8.00 | | 5.50 | | 13.00 | |
| Q1; Q3 | 1.00; 8.00 | | 8.00; 8.00 | | 1.00; 15.00 | | 12.00; 13.00 | |
| Min; Max | 1.0; 8.0 | | 8.0; 8.0 | | 0.0; 15.0 | | 4.0; 16.0 | |
| Change from baseline | | | | | | | | |
| Number (observed/imputed) | 1 | (1/0) | 1 | (1/0) | 6 | (5/1) | 5 | (5/0) |
| Mean (SD) | 8.00 | (NC) | 3.00 | (NC) | 4.33 | (6.77) | 7.80 | (5.89) |
| Median | 8.00 | | 3.00 | | 4.00 | | 9.00 | |
| Q1; Q3 | 8.00; 8.00 | | 3.00; 3.00 | | −2.00; 11.00 | | 5.00; 9.00 | |
| Min; Max | 8.0; 8.0 | | 3.0; 3.0 | | −3.0; 12.0 | | 0.0; 16.0 | |
| Percent change from baseline | | | | | | | | |
| Number (observed/imputed) | 0 | (0/0) | 1 | (1/0) | 6 | (5/1) | 4 | (4/0) |
| Mean (SD) | NC | (NC) | 60.00 | (NC) | 151.39 | (233.48) | 146.88 | (139.33) |
| Median | NC | | 60.00 | | 137.50 | | 143.75 | |
| Q1; Q3 | NC; NC | | 60.00; 60.00 | | −66.67; 400.00 | | 31.25; 262.50 | |
| Min; Max | NC; NC | | 60.0; 60.0 | | −100.0; 400.0 | | 0.0; 300.0 | |
| Week 8 | | | | | | | | |
| Number (observed/imputed) | 3 | (3/0) | 1 | (1/0) | 5 | (4/1) | 6 | (5/1) |
| Mean (SD) | 6.00 | (2.00) | 11.00 | (NC) | 9.20 | (7.98) | 11.00 | (4.00) |
| Median | 6.00 | | 11.00 | | 14.00 | | 11.50 | |
| Q1; Q3 | 4.00; 8.00 | | 11.00; 11.00 | | 1.00; 15.00 | | 10.00; 13.00 | |
| Min; Max | 4.0; 8.0 | | 11.0; 11.0 | | 0.0; 16.0 | | 4.0; 16.0 | |
| Change from baseline | | | | | | | | |
| Number (observed/imputed) | 1 | (1/0) | 1 | (1/0) | 5 | (4/1) | 6 | (5/1) |
| Mean (SD) | 4.00 | (NC) | 6.00 | (NC) | 6.60 | (7.57) | 6.17 | (6.01) |
| Median | 4.00 | | 6.00 | | 11.00 | | 5.00 | |
| Q1; Q3 | 4.00; 4.00 | | 6.00; 6.00 | | 0.00; 11.00 | | 2.00; 9.00 | |
| Min; Max | 4.0; 4.0 | | 6.0; 6.0 | | −3.0; 14.0 | | 0.0; 16.0 | |
| Percent change from baseline | | | | | | | | |
| Number (observed/imputed) | 0 | (0/0) | 1 | (1/0) | 5 | (4/1) | 5 | (4/1) |
| Mean (SD) | NC | (NC) | 120.00 | (NC) | 248.33 | (316.91) | 107.33 | (127.63) |
| Median | NC | | 120.00 | | 275.00 | | 25.00 | |
| Q1; Q3 | NC; NC | | 120.00; 120.00 | | 0.00; 366.67 | | 20.00; 225.00 | |
| Min; Max | NC; NC | | 120.0; 120.0 | | −100.0; 700.0 | | 0.0; 266.7 | |
| Week 12 | | | | | | | | |
| Number (observed/imputed) | 3 | (2/1) | 1 | (1/0) | 5 | (4/1) | 5 | (4/1) |
| Mean (SD) | 7.67 | (3.51) | 10.00 | (NC) | 9.20 | (7.33) | 11.80 | (4.71) |
| Median | 8.00 | | 10.00 | | 11.00 | | 12.00 | |
| Q1; Q3 | 4.00; 11.00 | | 10.00; 10.00 | | 2.00; 16.00 | | 12.00; 15.00 | |
| Min; Max | 4.0; 11.0 | | 10.0; 10.0 | | 1.0; 16.0 | | 4.0; 16.0 | |
| Change from baseline | | | | | | | | |
| Number (observed/imputed) | 1 | (1/0) | 1 | (1/0) | 5 | (4/1) | 5 | (4/1) |
| Mean (SD) | 8.00 | (NC) | 5.00 | (NC) | 6.60 | (6.84) | 8.00 | (6.20) |
| Median | 8.00 | | 5.00 | | 8.00 | | 9.00 | |
| Q1; Q3 | 8.00; 8.00 | | 5.00; 5.00 | | 0.00; 12.00 | | 4.00; 11.00 | |
| Min; Max | 8.0; 8.0 | | 5.0; 5.0 | | −1.0; 14.0 | | 0.0; 16.0 | |
| Percent change from baseline | | | | | | | | |
| Number (observed/imputed) | 0 | (0/0) | 1 | (1/0) | 5 | (4/1) | 4 | (3/1) |
| Mean (SD) | NC | (NC) | 100.00 | (NC) | 246.67 | (294.96) | 156.25 | (153.26) |
| Median | NC | | 100.00 | | 266.67 | | 162.50 | |
| Q1; Q3 | NC; NC | | 100.00; 100.00 | | 0.00; 300.00 | | 25.00; 287.50 | |
| Min; Max | NC; NC | | 100.0; 100.0 | | −33.3; 700.0 | | 0.0; 300.0 | |
| End of study | | | | | | | | |
| Number (observed/imputed) | 1 | | 1 | | 6 | | 6 | |
| Mean (SD) | 0.00 | (NC) | 5.00 | (NC) | 2.67 | (1.03) | 4.83 | (3.60) |
| Median | 0.00 | | 5.00 | | 3.00 | | 4.00 | |
| Q1; Q3 | 0.00; 0.00 | | 5.00; 5.00 | | 2.00; 3.00 | | 3.00; 8.00 | |
| Min; Max | 0.0; 0.0 | | 5.0; 5.0 | | 1.0; 4.0 | | 0.0; 10.0 | |
| Change from baseline | | | | | | | | |
| Number (observed/imputed) | 3 | (3/0) | 1 | (1/0) | 6 | (5/1) | 5 | (5/0) |
| Mean (SD) | 4.33 | (3.51) | 8.00 | (NC) | 7.00 | (7.18) | 11.60 | (4.51) |
| Median | 4.00 | | 8.00 | | 5.50 | | 13.00 | |
| Q1; Q3 | 1.00; 8.00 | | 8.00; 8.00 | | 1.00; 15.00 | | 12.00; 13.00 | |
| Min; Max | 1.0; 8.0 | | 8.0; 8.0 | | 0.0; 15.0 | | 4.0; 16.0 | |
| Percent change from baseline | | | | | | | | |
| Number (observed/imputed) | 1 | (1/0) | 1 | (1/0) | 6 | (5/1) | 5 | (5/0) |
| Mean (SD) | 8.00 | (NC) | 3.00 | (NC) | 4.33 | (6.77) | 7.80 | (5.89) |

| | | | | |
|---|---|---|---|---|
| Median | 8.00 | 3.00 | 4.00 | 9.00 |
| Q1; Q3 | 8.00; 8.00 | 3.00; 3.00 | −2.00; 11.00 | 5.00; 9.00 |
| Min; Max | 8.0; 8.0 | 3.0; 3.0 | −3.0; 12.0 | 0.0; 16.0 |

TABLE 46
Change and Percent Change from Baseline in UCT over Time up to Week 12 (ITT Population)

| | | | | | Change difference for | |
|---|---|---|---|---|---|---|
| | | | Post- | Change from | rilzabrutinib vs. placebo | |
| | | Baseline | baseline | baseline | LS Mean | |
| | n | Mean (SD) | Mean (SD) | LS Mean (SE)^{a} | (95% CI)^{a} | p-value^{a} |
| Week 4 | | | | | | |
| Rilzabrutinib 400 mg TID | 35 | 4.53 (3.50) | 10.03 (4.20) | 5.68 (0.68) | 2.34 (0.46, 4.22) | 0.0146 |
| (N = 41) | | | | | | |
| Rilzabrutinib 400 mg BID | 39 | 4.24 (3.06) | 8.56 (4.95) | 4.27 (0.65) | 0.94 (−0.87, 2.75) | 0.3089 |
| (N = 41) | | | | | | |
| Rilzabrutinib 400 mg QPM | 35 | 4.03 (2.16) | 6.40 (3.11) | 2.44 (0.68) | −0.89 (−2.79, 1.00) | 0.3558 |
| (N = 38) | | | | | | |
| Placebo (N = 40) | 33 | 4.17 (3.12) | 7.18 (4.07) | 3.33 (0.71) | | |
| Week 8 | | | | | | |
| Rilzabrutinib 400 mg TID | 36 | 4.53 (3.50) | 10.00 (3.85) | 5.80 (0.69) | 1.43 (−0.50, 3.37) | 0.1471 |
| (N = 41) | | | | | | |
| Rilzabrutinib 400 mg BID | 38 | 4.24 (3.06) | 10.00 (4.71) | 5.38 (0.68) | 1.01 (−0.90, 2.92) | 0.3016 |
| (N = 41) | | | | | | |
| Rilzabrutinib 400 mg QPM | 33 | 4.03 (2.16) | 6.97 (3.84) | 3.16 (0.71) | −1.21 (−3.16, 0.74) | 0.2226 |
| (N = 38) | | | | | | |
| Placebo (N = 40) | 34 | 4.17 (3.12) | 8.41 (4.25) | 4.37 (0.74) | | |
| Week 12 | | | | | | |
| Rilzabrutinib 400 mg TID | 35 | 4.53 (3.50) | 10.69 (4.25) | 6.53 (0.70) | 1.80 (−0.20, 3.79) | 0.0771 |
| (N = 41) | | | | | | |
| Rilzabrutinib 400 mg BID | 36 | 4.24 (3.06) | 9.92 (4.69) | 5.44 (0.71) | 0.71 (−1.27, 2.68) | 0.4839 |
| (N = 41) | | | | | | |
| Rilzabrutinib 400 mg QPM | 34 | 4.03 (2.16) | 6.97 (3.84) | 2.92 (0.76) | −1.82 (−3.87, 0.23) | 0.0812 |
| (N = 38) | | | | | | |
| Placebo (N = 40) | 33 | 4.17 (3.12) | 8.64 (4.14) | 4.74 (0.76) | | |

| | | % Change difference for | |
|---|---|---|---|
| | % Change | rilzabrutinib vs. placebo | |
| | from baseline | LS Mean | |
| | LS Mean (SE)^{a} | (95% CI)^{a} | p-value^{a} |
| Week 4 | | | |
| Rilzabrutinib 400 mg TID | 262.11 (48.72) | 122.56 (−10.01, 255.13) | 0.0699 |
| (N = 41) | | | |
| Rilzabrutinib 400 mg BID | 236.29 (42.88) | 96.74 (−24.75, 218.23) | 0.1185 |
| (N = 41) | | | |
| Rilzabrutinib 400 mg QPM | 95.52 (42.85) | −44.03 (−169.40, 81.34) | 0.4908 |
| (N = 38) | | | |
| Placebo (N = 40) | 139.55 (48.25) | | |
| Week 8 | | | |
| Rilzabrutinib 400 mg TID | 272.85 (43.60) | 108.30 (−18.30, 234.90) | 0.0935 |
| (N = 41) | | | |
| Rilzabrutinib 400 mg BID | 304.12 (43.11) | 139.58 (18.06, 261.09) | 0.0244 |
| (N = 41) | | | |
| Rilzabrutinib 400 mg QPM | 126.71 (44.63) | −37.84 (−167.44, 91.76) | 0.5667 |
| (N = 38) | | | |
| Placebo (N = 40) | 164.55 (48.15) | | |
| Week 12 | | | |
| Rilzabrutinib 400 mg TID | 322.21 (47.00) | 122.43 (−16.89, 261.74) | 0.0849 |
| (N = 41) | | | |
| Rilzabrutinib 400 mg BID | 309.12 (47.68) | 109.34 (−24.34, 243.02) | 0.1088 |
| (N = 41) | | | |
| Rilzabrutinib 400 mg QPM | 99.76 (47.53) | −100.02 (−238.68, 38.64) | 0.1572 |
| (N = 38) | | | |
| Placebo (N = 40) | 199.78 (53.12) | | |

^{a}Each of the imputed complete data were analyzed by fitting an ANCOVA model with the corresponding baseline value, intervention group and regions as covariates.

TABLE 47
Change and Percent Change from Baseline in UCT over Time
up to Week 12 (Omalizumab-Naïve Population)

| | | | | | Change difference for | |
|---|---|---|---|---|---|---|
| | | | Post- | Change from | rilzabrutinib vs. placebo | |
| | | Baseline | baseline | baseline | LS Mean | |
| | n | Mean (SD) | Mean (SD) | LS Mean (SE)^{a} | (95% CI)^{a} | p-value^{a} |
| Week 4 | | | | | | |
| Rilzabrutinib 400 mg TID | 30 | 4.47 (3.54) | 9.77 (4.17) | 5.47 (0.70) | 2.23 (0.33, 4.13) | 0.0216 |
| (N = 35) | | | | | | |
| Rilzabrutinib 400 mg BID | 33 | 4.51 (3.21) | 8.85 (4.53) | 4.51 (0.66) | 1.27 (−0.56, 3.11) | 0.1736 |
| (N = 35) | | | | | | |
| Rilzabrutinib 400 mg QPM | 34 | 4.00 (2.18) | 6.35 (3.14) | 2.36 (0.66) | −0.88 (−2.74, 0.99) | 0.3559 |
| (N = 37) | | | | | | |
| Placebo (N = 36) | 30 | 4.29 (3.08) | 7.47 (4.07) | 3.24 (0.70) | | |
| Week 8 | | | | | | |
| Rilzabrutinib 400 mg TID | 30 | 4.47 (3.54) | 9.80 (3.86) | 5.66 (0.71) | 1.19 (−0.75, 3.12) | 0.2295 |
| (N = 35) | | | | | | |
| Rilzabrutinib 400 mg BID | 33 | 4.51 (3.21) | 10.12 (4.20) | 5.61 (0.69) | 1.14 (−0.74, 3.02) | 0.2334 |
| (N = 35) | | | | | | |
| Rilzabrutinib 400 mg QPM | 32 | 4.00 (2.18) | 6.84 (3.84) | 2.98 (0.71) | −1.49 (−3.43, 0.45) | 0.1318 |
| (N = 37) | | | | | | |
| Placebo (N = 36) | 31 | 4.29 (3.08) | 8.65 (4.36) | 4.47 (0.72) | | |
| Week 12 | | | | | | |
| Rilzabrutinib 400 mg TID | 30 | 4.47 (3.54) | 10.50 (4.23) | 6.31 (0.76) | 1.58 (−0.47, 3.63) | 0.1307 |
| (N = 35) | | | | | | |
| Rilzabrutinib 400 mg BID | 31 | 4.51 (3.21) | 10.03 (4.29) | 5.52 (0.74) | 0.80 (−1.21, 2.81) | 0.4374 |
| (N = 35) | | | | | | |
| Rilzabrutinib 400 mg QPM | 33 | 4.00 (2.18) | 6.88 (3.86) | 2.77 (0.75) | −1.96 (−3.99, 0.08) | 0.0595 |
| (N = 37) | | | | | | |
| Placebo (N = 36) | 30 | 4.29 (3.08) | 8.73 (4.23) | 4.73 (0.75) | | |

| | | % Change difference for | |
|---|---|---|---|
| | % Change | rilzabrutinib vs. placebo | |
| | from baseline | LS Mean | |
| | LS Mean (SE)^{a} | (95% CI)^{a} | p-value^{a} |
| Week 4 | | | |
| Rilzabrutinib 400 mg TID | 267.24 (49.68) | 124.43 (−9.74, 258.61) | 0.0691 |
| (N = 35) | | | |
| Rilzabrutinib 400 mg BID | 263.00 (44.80) | 120.19 (−1.20, 241.59) | 0.0523 |
| (N = 35) | | | |
| Rilzabrutinib 400 mg QPM | 94.30 (43.68) | −48.50 (−174.55, 77.55) | 0.4504 |
| (N = 37) | | | |
| Placebo (N = 36) | 142.81 (47.73) | | |
| Week 8 | | | |
| Rilzabrutinib 400 mg TID | 272.13 (49.40) | 102.55 (−31.38, 236.49) | 0.1332 |
| (N = 35) | | | |
| Rilzabrutinib 400 mg BID | 328.12 (46.96) | 158.55 (28.05, 289.04) | 0.0173 |
| (N = 35) | | | |
| Rilzabrutinib 400 mg QPM | 121.09 (44.66) | −48.49 (−177.78, 80.81) | 0.4620 |
| (N = 37) | | | |
| Placebo (N = 36) | 169.58 (49.20) | | |
| Week 12 | | | |
| Rilzabrutinib 400 mg TID | 329.00 (51.79) | 131.76 (−18.16, 281.68) | 0.0848 |
| (N = 35) | | | |
| Rilzabrutinib 400 mg BID | 344.03 (53.11) | 146.79 (5.56, 288.02) | 0.0417 |
| (N = 35) | | | |
| Rilzabrutinib 400 mg QPM | 91.29 (48.06) | −105.95 (−247.17, 35.27) | 0.1412 |
| (N = 37) | | | |
| Placebo (N = 36) | 197.24 (53.74) | | |

^{a}Each of the imputed complete data were analyzed by fitting an ANCOVA model with the corresponding baseline value, intervention group and regions as covariates.

### Quality of Life

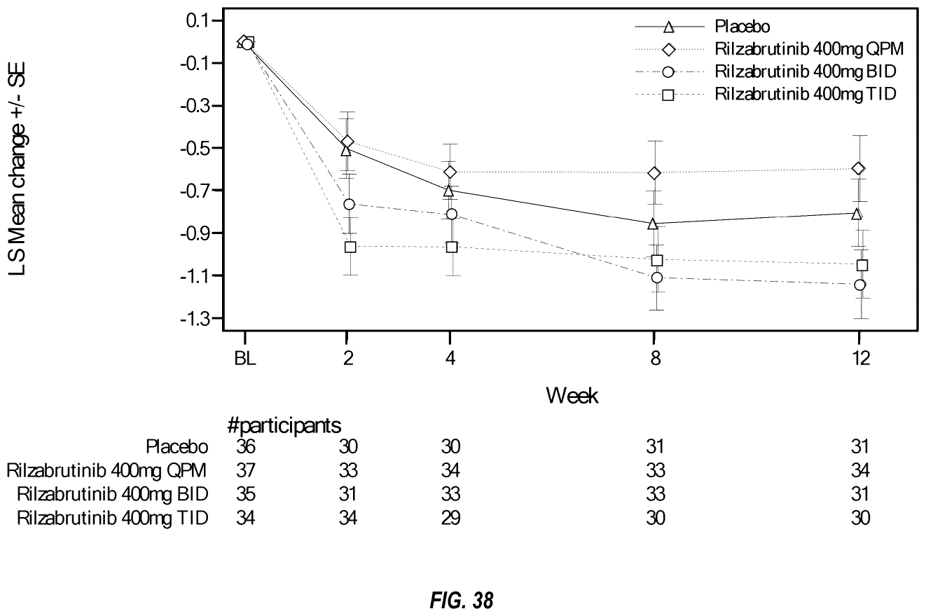

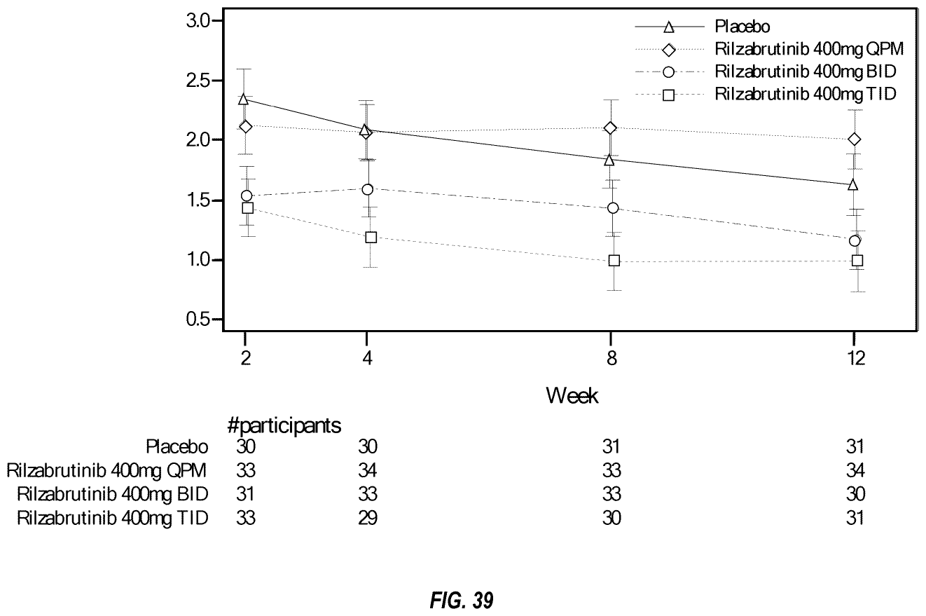

Patients receiving 400 mg TID rilzabrutinib experienced an improvement in quality of life as measured by PGIS and PGIC (see FIGS. 38-39 and Tables 48-52).

TABLE 48
Summary of PGIS by Visit in the Double-Blind Period (Omalizumab-Naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| PGIS | (N = 36) | (N = 37) | (N = 35) | (N = 35) |

Baseline

| Number | 36 | | 37 | | 35 | | 34 | |
|---|---|---|---|---|---|---|---|---|
| Mean (SD) | 2.39 | (0.60) | 2.51 | (0.61) | 2.20 | (0.63) | 2.32 | (0.68) |

| | | | | |
|---|---|---|---|---|
| Median | 2.00 | 3.00 | 2.00 | 2.00 |
| Q1; Q3 | 2.00; 3.00 | 2.00; 3.00 | 2.00; 3.00 | 2.00; 3.00 |
| Min; Max | 1.0; 3.0 | 1.0; 3.0 | 1.0; 3.0 | 0.0; 3.0 |

Week 2

| | | | | | | | | |
|---|---|---|---|---|---|---|---|---|
| Number (observed/imputed) | 30 | (30/0) | 33 | (33/0) | 31 | (31/0) | 34 | (34/0) |
| Mean (SD) | 1.90 | (0.71) | 2.00 | (0.71) | 1.55 | (0.93) | 1.38 | (0.85) |

| | | | | |
|---|---|---|---|---|
| Median | 2.00 | 2.00 | 2.00 | 1.00 |
| Q1; Q3 | 1.00; 2.00 | 2.00; 2.00 | 1.00; 2.00 | 1.00; 2.00 |
| Min; Max | 1.0; 3.0 | 1.0; 3.0 | 0.0; 3.0 | 0.0; 3.0 |

Change from baseline

| | | | | | | | | |
|---|---|---|---|---|---|---|---|---|
| Number (observed/imputed) | 30 | (30/0) | 33 | (33/0) | 31 | (31/0) | 34 | (34/0) |
| Mean (SD) | −0.43 | (0.63) | −0.48 | (0.67) | −0.65 | (1.02) | −0.94 | (1.01) |

| | | | | |
|---|---|---|---|---|
| Median | 0.00 | 0.00 | 0.00 | 1.00 |
| Q1; Q3 | −1.00; 0.00 | −1.00; 0.00 | −2.00; 0.00 | −2.00; 0.00 |
| Min; Max | −2.0; 1.0 | −2.0; 1.0 | −3.0; 1.0 | −3.0; 2.0 |

Percent change from baseline

| | | | | | | | | |
|---|---|---|---|---|---|---|---|---|
| Number (observed/imputed) | 30 | (30/0) | 33 | (33/0) | 31 | (31/0) | 33 | (33/0) |
| Mean (SD) | −17.22 | (26.07) | −17.68 | (25.66) | −23.12 | (50.49) | −41.92 | (35.63) |

| | | | | |
|---|---|---|---|---|
| Median | 0.00 | 0.00 | 0.00 | −50.00 |
| Q1; Q3 | −33.33; 0.00 | −33.33; 0.00 | −66.67; 0.00 | −66.67; 0.00 |
| Min; Max | −66.7; 50.0 | −66.7; 50.0 | −100.0; 100.0 | −100.0; 50.0 |

Week 4

| | | | | | | | | |
|---|---|---|---|---|---|---|---|---|
| Number (observed/imputed) | 30 | (30/0) | 34 | (33/1) | 33 | (33/0) | 29 | (29/0) |
| Mean (SD) | 1.63 | (0.76) | 1.88 | (0.81) | 1.48 | (0.87) | 1.34 | (0.77) |

| | | | | |
|---|---|---|---|---|
| Median | 2.00 | 2.00 | 2.00 | 1.00 |
| Q1; Q3 | 1.00; 2.00 | 1.00; 2.00 | 1.00; 2.00 | 1.00; 2.00 |
| Min; Max | 0.0; 3.0 | 0.0; 3.0 | 0.0; 3.0 | 0.0; 3.0 |

Change from baseline

| | | | | | | | | |
|---|---|---|---|---|---|---|---|---|
| Number (observed/imputed) | 30 | (30/0) | 34 | (33/1) | 33 | (33/0) | 29 | (29/0) |
| Mean (SD) | −0.67 | (0.76) | −0.65 | (0.73) | −0.70 | (0.92) | −1.07 | (0.80) |

| | | | | |
|---|---|---|---|---|
| Median | −1.00 | −0.50 | 0.00 | 1.00 |
| Q1; Q3 | −1.00; 0.00 | −1.00; 0.00 | −1.00; 0.00 | −2.00; −1.00 |
| Min; Max | −2.0; 1.0 | −2.0; 0.0 | −3.0; 1.0 | −3.0; 0.0 |

Percent change from baseline

| | | | | | | | | |
|---|---|---|---|---|---|---|---|---|
| Number (observed/imputed) | 30 | (30/0) | 34 | (33/1) | 33 | (33/0) | 29 | (29/0) |
| Mean (SD) | −27.22 | (31.71) | −25.49 | (29.08) | −27.78 | (43.43) | −43.10 | (30.71) |

| | | | | |
|---|---|---|---|---|
| Median | −33.33 | −16.67 | 0.00 | −50.00 |
| Q1; Q3 | −50.00; 0.00 | −50.00; 0.00 | −50.00; 0.00 | −66.67; −33.33 |
| Min; Max | −100.0; 50.0 | −100.0; 0.0 | −100.0; 100.0 | −100.0; 0.0 |

Week 8

| | | | | | | | | |
|---|---|---|---|---|---|---|---|---|
| Number (observed/imputed) | 31 | (30/1) | 33 | (30/3) | 33 | (32/1) | 30 | (30/0) |
| Mean (SD) | 1.52 | (0.89) | 1.88 | (0.82) | 1.18 | (0.98) | 1.33 | (0.96) |

| | | | | |
|---|---|---|---|---|
| Median | 1.00 | 2.00 | 1.00 | 1.00 |
| Q1; Q3 | 1.00; 2.00 | 1.00; 2.00 | 0.00; 2.00 | 1.00; 2.00 |
| Min; Max | 0.0; 3.0 | 0.0; 3.0 | 0.0; 3.0 | 0.0; 3.0 |

Change from baseline

| | | | | | | | | |
|---|---|---|---|---|---|---|---|---|
| Number (observed/imputed) | 31 | (30/1) | 33 | (30/3) | 33 | (32/1) | 30 | (30/0) |
| Mean (SD) | −0.81 | (0.91) | 0.67 | (0.74) | −1.00 | (0.97) | −1.13 | (0.86) |

| | | | | |
|---|---|---|---|---|
| Median | −1.00 | −1.00 | −1.00 | −1.00 |
| Q1; Q3 | −1.00; 0.00 | −1.00; 0.00 | −2.00; 0.00 | −2.00; −1.00 |
| Min; Max | −3.0; 1.0 | −2.0; 1.0 | −3.0; 1.0 | −3.0; 0.0 |

Percent change from baseline

| | | | | | | | | |
|---|---|---|---|---|---|---|---|---|
| Number (observed/imputed) | 31 | (30/1) | 33 | (30/3) | 33 | (32/1) | 30 | (30/0) |
| Mean (SD) | −32.80 | (36.64) | −25.25 | (35.63) | −44.95 | (43.60) | 47.22 | (34.49) |

| | | | | |
|---|---|---|---|---|
| Median | −33.33 | −33.33 | −50.00 | −50.00 |
| Q1; Q3 | −50.00; 0.00 | −50.00; 0.00 | −100.00; 0.00 | −66.67; −33.33 |
| Min; Max | −100.0; 50.0 | −100.0; 100.0 | −100.0; 50.0 | −100.0; 0.0 |

Week 12

| | | | | | | | | |
|---|---|---|---|---|---|---|---|---|
| Number (observed/imputed) | 31 | (30/1) | 34 | (31/3) | 31 | (29/2) | 30 | (29/1) |
| Mean (SD) | 1.58 | (0.92) | 1.88 | (0.81) | 1.13 | (1.06) | 1.27 | (0.91) |

| | | | | |
|---|---|---|---|---|
| Median | 1.00 | 2.00 | 1.00 | 1.00 |
| Q1; Q3 | 1.00; 2.00 | 1.00; 2.00 | 0.00; 2.00 | 1.00; 2.00 |
| Min; Max | 0.0; 3.0 | 0.0; 3.0 | 0.0; 3.0 | 0.0; 3.0 |

Change from baseline

| | | | | | | | | |
|---|---|---|---|---|---|---|---|---|
| Number (observed/imputed) | 31 | (30/1) | 34 | (31/3) | 31 | (29/2) | 30 | (29/1) |
| Mean (SD) | −0.81 | (0.95) | −0.68 | (0.64) | −1.03 | (1.17) | −1.17 | (0.91) |

| | | | | |
|---|---|---|---|---|
| Median | −1.00 | −1.00 | −1.00 | −1.00 |
| Q1; Q3 | −2.00; 0.00 | −1.00; 0.00 | −2.00; 0.00 | −2.00; 0.00 |
| Min; Max | 2.0; 1.0 | −2.0; 0.0 | −3.0; 1.0 | −3.0; 0.0 |

Percent change from baseline

| | | | | | | | | |
|---|---|---|---|---|---|---|---|---|
| Number (observed/imputed) | 31 | (30/1) | 34 | (31/3) | 31 | (29/2) | 30 | (29/1) |
| Mean (SD) | −32.26 | (39.89) | −27.45 | (26.87) | −43.55 | (55.56) | −47.22 | (35.85) |

| | | | | |
|---|---|---|---|---|
| Median | −33.33 | −33.33 | −50.00 | −50.00 |
| Q1; Q3 | −66.67; 0.00 | −50.00; 0.00 | −100.00; 0.00 | −66.67; 0.00 |
| Min; Max | −100.0; 50.0 | −100.0; 0.0 | −100.0; 100.0 | −100.0; 0.0 |

TABLE 49
Change and Percent Change from Baseline in PGIS over Time
up to Week 12 (Omalizumab-Naïve Population)

| | | | | | Change difference for | |
|---|---|---|---|---|---|---|
| | | | Post- | Change from | rilzabrutinib vs. placebo | |
| | | Baseline | baseline | baseline | LS Mean | |
| | n | Mean (SD) | Mean (SD) | LS Mean (SE)^{a} | (95% CI)^{a} | p-value^{a} |
| Week 2 | | | | | | |
| Rilzabrutinib 400 mg TID | 34 | 2.32 (0.68) | 1.38 (0.85) | −0.96 (0.13) | −0.46 (−0.83, −0.09) | 0.0148 |
| (N = 35) | | | | | | |
| Rilzabrutinib 400 mg BID | 31 | 2.20 (0.63) | 1.55 (0.93) | −0.76 (0.14) | −0.26 (−0.63, 0.11) | 0.1666 |
| (N = 35) | | | | | | |
| Rilzabrutinib 400 mg QPM | 33 | 2.51 (0.61) | 2.00 (0.71) | −0.47 (0.14) | 0.03 (−0.34, 0.41) | 0.8643 |
| (N = 37) | | | | | | |
| Placebo (N = 36) | 30 | 2.39 (0.60) | 1.90 (0.71) | −0.50 (0.14) | | |
| Week 4 | | | | | | |
| Rilzabrutinib 400 mg TID | 29 | 2.32 (0.68) | 1.34 (0.77) | −0.96 (0.13) | −0.27 (−0.62, 0.09) | 0.1413 |
| (N = 35) | | | | | | |
| Rilzabrutinib 400 mg BID | 33 | 2.20 (0.63) | 1.48 (0.87) | −0.81 (0.13) | −0.12 (−0.48, 0.25) | 0.5305 |
| (N = 35) | | | | | | |
| Rilzabrutinib 400 mg QPM | 34 | 2.51 (0.61) | 1.88 (0.81) | −0.61 (0.13) | 0.09 (−0.27, 0.44) | 0.6363 |
| (N = 37) | | | | | | |
| Placebo (N = 36) | 30 | 2.39 (0.60) | 1.63 (0.76) | −0.70 (0.14) | | |
| Week 8 | | | | | | |
| Rilzabrutinib 400 mg TID | 30 | 2.32 (0.68) | 1.33 (0.96) | −1.02 (0.15) | −0.17 (−0.58, 0.25) | 0.4296 |
| (N = 35) | | | | | | |
| Rilzabrutinib 400 mg BID | 33 | 2.20 (0.63) | 1.18 (0.98) | −1.11 (0.15) | −0.25 (−0.67, 0.16) | 0.2338 |
| (N = 35) | | | | | | |
| Rilzabrutinib 400 mg QPM | 33 | 2.51 (0.61) | 1.88 (0.82) | −0.61 (0.15) | 0.24 (−0.17, 0.65) | 0.2464 |
| (N = 37) | | | | | | |
| Placebo (N = 36) | 31 | 2.39 (0.60) | 1.52 (0.89) | −0.85 (0.16) | | |
| Week 12 | | | | | | |
| Rilzabrutinib 400 mg TID | 30 | 2.32 (0.68) | 1.27 (0.91) | −1.04 (0.16) | −0.24 (−0.67, 0.19) | 0.2700 |
| (N = 35) | | | | | | |
| Rilzabrutinib 400 mg BID | 31 | 2.20 (0.63) | 1.13 (1.06) | −1.14 (0.16) | −0.34 (−0.77, 0.10) | 0.1301 |
| (N = 35) | | | | | | |
| Rilzabrutinib 400 mg QPM | 34 | 2.51 (0.61) | 1.88 (0.81) | −0.59 (0.15) | 0.21 (−0.22, 0.63) | 0.3380 |
| (N = 37) | | | | | | |
| Placebo (N = 36) | 31 | 2.39 (0.60) | 1.58 (0.92) | −0.80 (0.16) | | |

| | | % Change difference for | |
|---|---|---|---|
| | % Change | rilzabrutinib vs. placebo | |
| | from baseline | LS Mean | |
| | LS Mean (SE)^{a} | (95% CI)^{a} | p-value^{a} |
| Week 2 | | | |
| Rilzabrutinib 400 mg TID | −40.66 (6.06) | −22.00 (−38.77, −5.22) | 0.0102 |
| (N = 35) | | | |
| Rilzabrutinib 400 mg BID | −26.23 (6.28) | −7.56 (−24.17, 9.05) | 0.3723 |
| (N = 35) | | | |
| Rilzabrutinib 400 mg QPM | −17.80 (6.05) | 0.86 (−15.86, 17.59) | 0.9195 |
| (N = 37) | | | |
| Placebo (N = 36) | −18.67 (6.28) | | |
| Week 4 | | | |
| Rilzabrutinib 400 mg TID | −41.04 (6.24) | −12.52 (−29.03, 4.00) | 0.1374 |
| (N = 35) | | | |
| Rilzabrutinib 400 mg BID | −29.83 (5.99) | −1.31 (−17.26, 14.65) | 0.8725 |
| (N = 35) | | | |
| Rilzabrutinib 400 mg QPM | −25.46 (5.77) | 3.06 (−12.83, 18.95) | 0.7058 |
| (N = 37) | | | |
| Placebo (N = 36) | −28.52 (5.99) | | |
| Week 8 | | | |
| Rilzabrutinib 400 mg TID | −45.65 (7.05) | −11.39 (−30.26, 7.48) | 0.2367 |
| (N = 35) | | | |
| Rilzabrutinib 400 mg BID | −46.44 (6.74) | −12.18 (−30.63, 6.27) | 0.1958 |
| (N = 35) | | | |
| Rilzabrutinib 400 mg QPM | −24.99 (6.69) | 9.27 (−9.09, 27.62) | 0.3223 |
| (N = 37) | | | |
| Placebo (N = 36) | −34.26 (7.00) | | |
| Week 12 | | | |
| Rilzabrutinib 400 mg TID | −44.96 (7.44) | −12.67 (−32.97, 7.63) | 0.2211 |
| (N = 35) | | | |
| Rilzabrutinib 400 mg BID | −45.76 (7.46) | −13.46 (−33.69, 6.76) | 0.1919 |
| (N = 35) | | | |
| Rilzabrutinib 400 mg QPM | −26.42 (7.16) | 5.87 (−13.65, 25.38) | 0.5555 |
| (N = 37) | | | |
| Placebo (N = 36) | −32.29 (7.54) | | |

^{a}Each of the imputed complete data were analyzed by fitting an ANCOVA model with the corresponding baseline value, intervention group and regions as covariates.

TABLE 50
Categorical Summary of PGIS by Visit up to Week
12 (Omalizumab-Naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| PGIS | (N = 36) | (N = 37) | (N = 35) | (N = 35) |

Baseline

| | | | | |
|---|---|---|---|---|
| Number | 36 | 37 | 35 | 34 |
| None | 0 | 0 | 0 | 1 (2.9) |
| Mild | 2 (5.6) | 2 (5.4) | 4 (11.4) | 1 (2.9) |
| Moderate | 18 (50.0) | 14 (37.8) | 20 (57.1) | 18 (52.9) |
| Severe | 16 (44.4) | 21 (56.8) | 11 (31.4) | 14 (41.2) |

Week 2

| | | | | |
|---|---|---|---|---|
| Number (observed/imputed) | 30 (30/0) | 33 (33/0) | 31 (31/0) | 34 (34/0) |
| None | 0 | 0 | 5 (16.1) | 4 (11.8) |
| Mild | 9 (30.0) | 8 (24.2) | 8 (25.8) | 17 (50.0) |
| Moderate | 15 (50.0) | 17 (51.5) | 14 (45.2) | 9 (26.5) |
| Severe | 6 (20.0) | 8 (24.2) | 4 (12.9) | 4 (11.8) |

Week 4

| | | | | |
|---|---|---|---|---|
| Number (observed/imputed) | 30 (30/0) | 34 (33/1) | 33 (33/0) | 29 (29/0) |
| None | 1 (3.3) | 1 (2.9) | 5 (15.2) | 3 (10.3) |
| Mild | 13 (43.3) | 10 (29.4) | 10 (30.3) | 15 (51.7) |
| Moderate | 12 (40.0) | 15 (44.1) | 15 (45.5) | 9 (31.0) |
| Severe | 4 (13.3) | 8 (23.5) | 3 (9.1) | 2 (6.9) |

Week 8

| | | | | |
|---|---|---|---|---|
| Number (observed/imputed) | 31 (30/1) | 33 (30/3) | 33 (32/1) | 30 (30/0) |
| None | 3 (9.7) | 1 (3.0) | 9 (27.3) | 6 (20.0) |
| Mild | 14 (45.2) | 10 (30.3) | 13 (39.4) | 12 (40.0) |
| Moderate | 9 (29.0) | 14 (42.4) | 7 (21.2) | 8 (26.7) |
| Severe | 5 (16.1) | 8 (24.2) | 4 (12.1) | 4 (13.3) |

Week 12

| | | | | |
|---|---|---|---|---|
| Number (observed/imputed) | 31 (30/1) | 34 (31/3) | 31 (29/2) | 30 (29/1) |
| None | 3 (9.7) | 1 (2.9) | 12 (38.7) | 6 (20.0) |
| Mild | 13 (41.9) | 10 (29.4) | 6 (19.4) | 13 (43.3) |
| Moderate | 9 (29.0) | 15 (44.1) | 10 (32.3) | 8 (26.7) |
| Severe | 6 (19.4) | 8 (23.5) | 3 (9.7) | 3 (10.0) |

TABLE 51
Summary of PGIC by Visit in the Double-Blind Period (Omalizumab-Naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| PGIC | (N = 36) | (N = 37) | (N = 35) | (N = 35) |

Week 2

| Number (observed/imputed) | 30 | (30/0) | 33 | (33/0) | 31 | (31/0) | 33 | (33/0) |
|---|---|---|---|---|---|---|---|---|
| Mean (SD) | 2.50 | (1.25) | 2.33 | (1.24) | 1.61 | (1.63) | 1.42 | (1.52) |
| Median | 3.00 | | 3.00 | | 1.00 | | 1.00 | |
| Q1; Q3 | 2.00; 3.00 | | 2.00; 3.00 | | 0.00; 3.00 | | 0.00; 2.00 | |
| Min; Max | 0.0; 6.0 | | 0.0; 6.0 | | 0.0; 6.0 | | 0.0; 6.0 | |
| LS Mean (SE) ^{a} | 2.34 | (0.25) | 2.12 | (0.24) | 1.53 | (0.25) | 1.43 | (0.24) |
| LS Mean Diff vs. placebo (95% CI) ^{a} | | | −0.22 | (−0.89, 0.45) | −0.81 | (−1.47, −0.15) | −0.91 | (−1.59, −0.23) |

| | | | |
|---|---|---|---|
| P-value vs. placebo ^{a} | 0.5204 | 0.0166 | 0.0085 |

Week 4

| Number (observed/imputed) | 30 | (30/0) | 34 | (33/1) | 33 | (33/0) | 29 | (29/0) |
|---|---|---|---|---|---|---|---|---|
| Mean (SD) | 2.13 | (1.31) | 2.24 | (1.54) | 1.67 | (1.49) | 1.14 | (1.25) |
| Median | 2.00 | | 2.00 | | 1.00 | | 1.00 | |
| Q1; Q3 | 1.00; 3.00 | | 1.00; 3.00 | | 0.00; 3.00 | | 0.00; 2.00 | |
| Min; Max | 0.0; 5.0 | | 0.0; 6.0 | | 0.0; 6.0 | | 0.0; 4.0 | |
| LS Mean (SE) ^{a} | 2.09 | (0.25) | 2.06 | (0.24) | 1.59 | (0.24) | 1.18 | (0.25) |
| LS Mean Diff vs. placebo (95% CI) ^{a} | | | −0.02 | (−0.67, 0.62) | −0.49 | (−1.15, 0.16) | −0.90 | (−1.58, −0.22) |

| | | | |
|---|---|---|---|
| P-value vs. placebo ^{a} | 0.9433 | 0.1411 | 0.0092 |

Week 8

| Number (observed/imputed) | 31 | (30/1) | 33 | (30/3) | 33 | (32/1) | 30 | (30/0) |
|---|---|---|---|---|---|---|---|---|
| Mean (SD) | 1.84 | (1.07) | 2.21 | (1.41) | 1.42 | (1.62) | 0.90 | (1.12) |
| Median | 2.00 | | 2.00 | | 1.00 | | 1.00 | |
| Q1; Q3 | 1.00; 3.00 | | 1.00; 3.00 | | 0.00; 3.00 | | 0.00; 1.00 | |
| Min; Max | 0.0; 4.0 | | 0.0; 6.0 | | 0.0; 6.0 | | 0.0; 4.0 | |
| LS Mean (SE) ^{a} | 1.84 | (0.24) | 2.10 | (0.23) | 1.43 | (0.24) | 0.98 | (0.24) |
| LS Mean Diff vs. placebo (95% CI) ^{a} | | | 0.27 | (−0.37, 0.90) | −0.41 | (−1.05, 0.23) | −0.86 | (−1.50, −0.21) |

| | | | |
|---|---|---|---|
| P−value vs. placebo ^{a} | 0.4112 | 0.2098 | 0.0093 |

Week 12

| Number (observed/imputed) | 31 | (30/1) | 34 | (31/3) | 30 | (28/2) | 31 | (29/2) |
|---|---|---|---|---|---|---|---|---|
| Mean (SD) | 1.58 | (1.15) | 2.12 | (1.49) | 1.03 | (1.40) | 0.97 | (1.30) |
| Median | 2.00 | | 2.00 | | 0.50 | | 0.00 | |
| Q1; Q3 | 0.00; 3.00 | | 1.00; 3.00 | | 0.00; 1.00 | | 0.00; 2.00 | |
| Min; Max | 0.0; 3.0 | | 0.0; 6.0 | | 0.0; 5.0 | | 0.0; 4.0 | |
| LS Mean (SE) ^{a} | 1.62 | (0.26) | 2.00 | (0.25) | 1.17 | (0.25) | 0.98 | (0.25) |
| LS Mean Diff vs. placebo (95% CI) ^{a} | | | 0.38 | (−0.30, 1.05) | −0.46 | (−1.14, 0.23) | −0.64 | (−1.34, 0.05) |

| | | | |
|---|---|---|---|
| P-value vs. placebo ^{a} | 0.2716 | 0.1904 | 0.0703 |

^{a} Data were analyzed by fitting an ANCOVA model with the intervention group and regions as covariates.

TABLE 52
Categorical Summary of PGIC by Visit up to Week
12 (Omalizumab-Naïve Population)

| | | | | |
|---|---|---|---|---|
| | | Rilzabrutinib | Rilzabrutinib | Rilzabrutinib |
| | Placebo | 400 mg QPM | 400 mg BID | 400 mg TID |
| PGIC | (N = 36) | (N = 37) | (N = 35) | (N = 35) |

Week 2

| | | | | |
|---|---|---|---|---|
| Number (observed/imputed) | 30 (30/0) | 33 (33/0) | 31 (31/0) | 33 (33/0) |
| Very much better | 3 (10.0) | 3 (9.1) | 11 (35.5) | 12 (36.4) |
| Moderately better | 1 (3.3) | 5 (15.2) | 5 (16.1) | 7 (21.2) |
| A little better | 9 (30.0) | 7 (21.2) | 6 (19.4) | 7 (21.2) |
| No change | 15 (50.0) | 16 (48.5) | 6 (19.4) | 5 (15.2) |
| A little worse | 0 | 1 (3.0) | 1 (3.2) | 0 |
| Moderately worse | 1 (3.3) | 0 | 1 (3.2) | 1 (3.0) |
| Very much worse | 1 (3.3) | 1 (3.0) | 1 (3.2) | 1 (3.0) |

Week 4

| | | | | |
|---|---|---|---|---|
| Number (observed/imputed) | 30 (30/0) | 34 (33/1) | 33 (33/0) | 29 (29/0) |
| Very much better | 4 (13.3) | 6 (17.6) | 9 (27.3) | 13 (44.8) |
| Moderately better | 5 (16.7) | 4 (11.8) | 8 (24.2) | 5 (17.2) |
| A little better | 8 (26.7) | 10 (29.4) | 6 (18.2) | 6 (20.7) |
| No change | 11 (36.7) | 7 (20.6) | 7 (21.2) | 4 (13.8) |
| A little worse | 0 | 5 (14.7) | 2 (6.1) | 1 (3.4) |
| Moderately worse | 2 (6.7) | 1 (2.9) | 0 | 0 |
| Very much worse | 0 | 1 (2.9) | 1 (3.0) | 0 |

Week 8

| | | | | |
|---|---|---|---|---|
| Number (observed/imputed) | 31 (30/1) | 33 (30/3) | 33 (32/1) | 30 (30/0) |
| Very much better | 4 (12.9) | 4 (12.1) | 13 (39.4) | 14 (46.7) |
| Moderately better | 7 (22.6) | 6 (18.2) | 8 (24.2) | 10 (33.3) |
| A little better | 11 (35.5) | 9 (27.3) | 3 (9.1) | 2 (6.7) |
| No change | 8 (25.8) | 10 (30.3) | 6 (18.2) | 3 (10.0) |
| A little worse | 1 (3.2) | 2 (6.1) | 1 (3.0) | 1 (3.3) |
| Moderately worse | 0 | 1 (3.0) | 1 (3.0) | 0 |
| Very much worse | 0 | 1 (3.0) | 1 (3.0) | 0 |

Week 12

| | | | | |
|---|---|---|---|---|
| Number (observed/imputed) | 31 (30/1) | 34 (31/3) | 30 (28/2) | 31 (29/2) |
| Very much better | 8 (25.8) | 6 (17.6) | 15 (50.0) | 17 (54.8) |
| Moderately better | 5 (16.1) | 6 (17.6) | 8 (26.7) | 5 (16.1) |
| A little better | 10 (32.3) | 7 (20.6) | 1 (3.3) | 4 (12.9) |
| No change | 8 (25.8) | 11 (32.4) | 4 (13.3) | 3 (9.7) |
| A little worse | 0 | 2 (5.9) | 1 (3.3) | 2 (6.5) |
| Moderately worse | 0 | 1 (2.9) | 1 (3.3) | 0 |
| Very much worse | | 1 (2.9) | 0 | 0 |

### Biomarkers

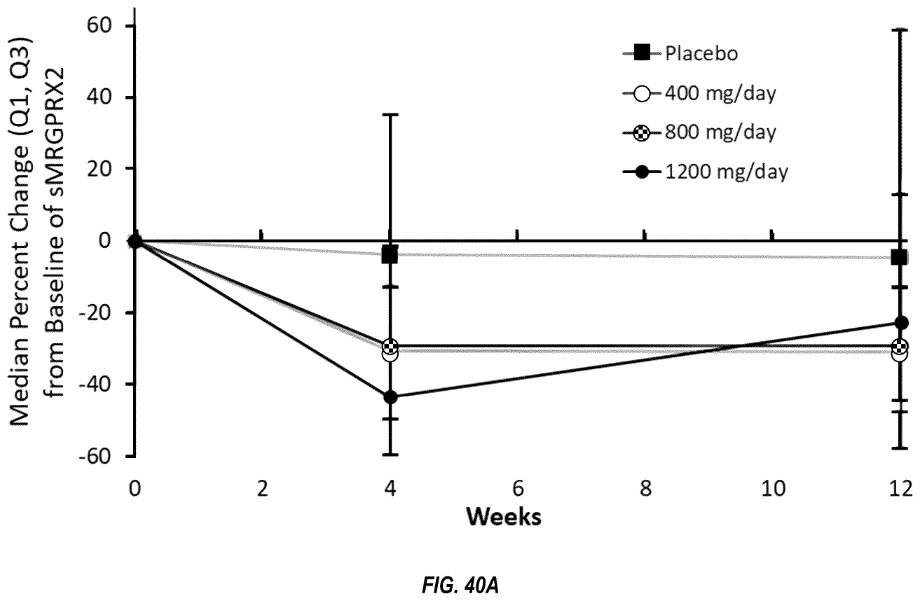

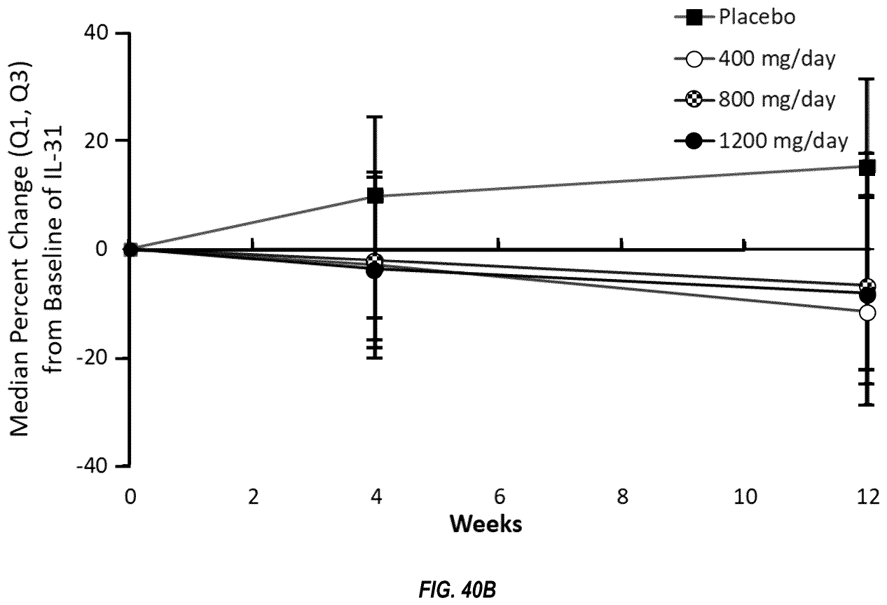

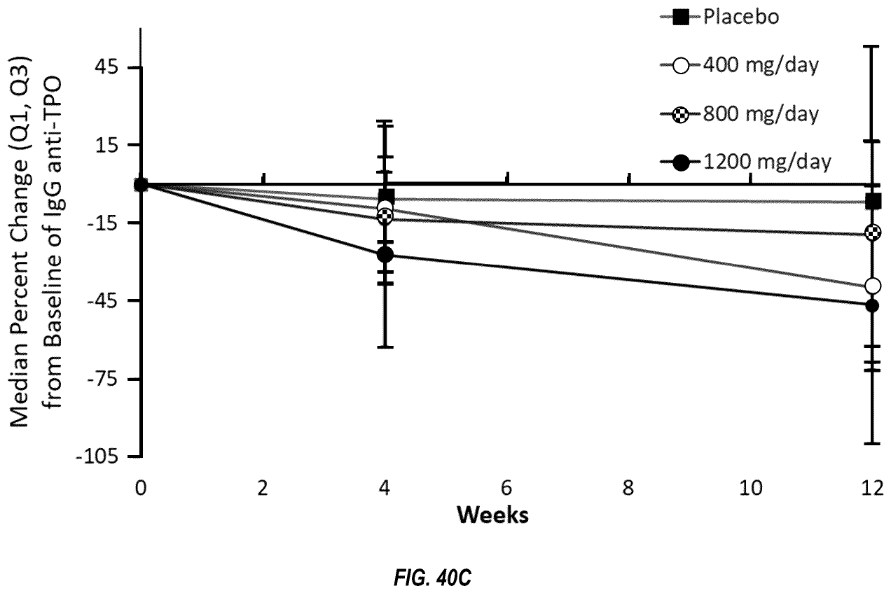

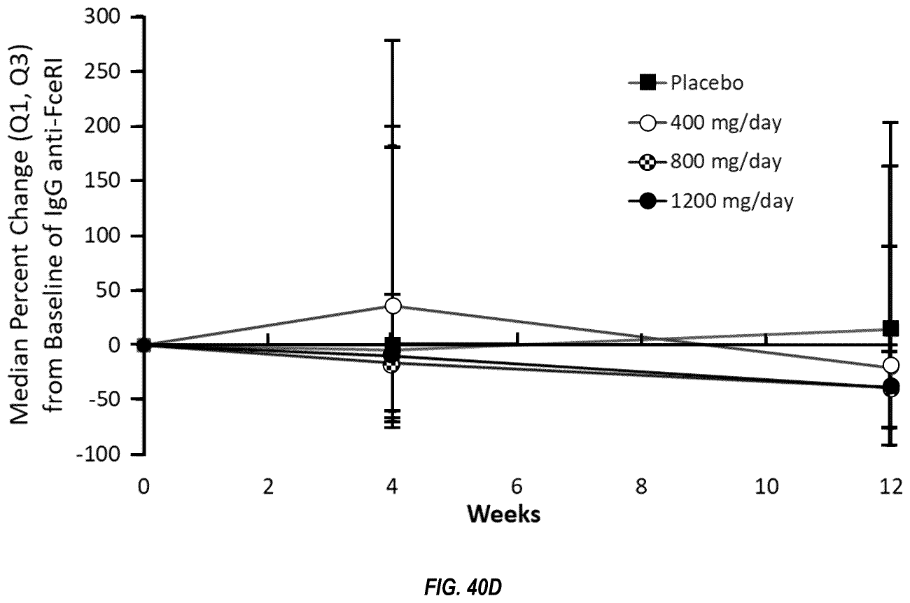

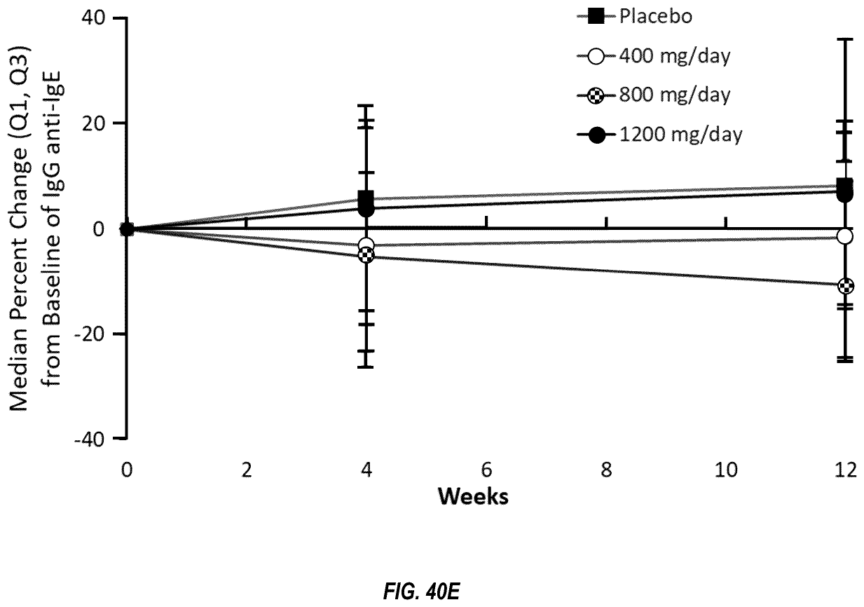

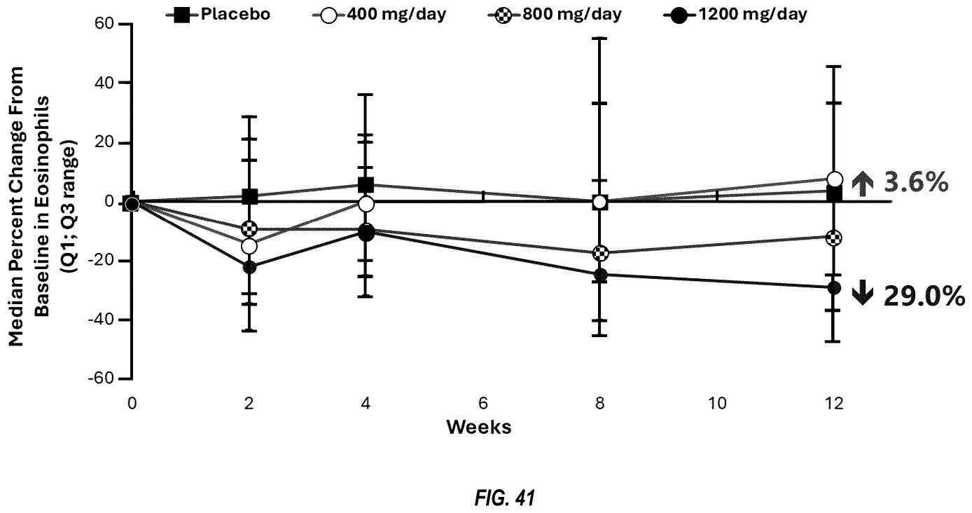

Changes in disease-relevant biomarkers were assessed over the course of the 12-Week double-blind phase (see FIG. 40). Biomarkers included sMRGPRX2, IgE anti-TPO, IgG anti-FCεRI, IL-31, IgM, IgE, IgG, IgG anti-IgE, and white blood cell levels. Consistent with the effects of rilzabrutinib on other aspects of CSU, patients receiving rilzabrutinib achieved a reduction in levels of sMRGPRX2 (a mast cell receptor associated with disease activity in CSU), IgE anti-TPO (a marker of Type IIb autoimmune CSU), IgG anti-FCεRI (a marker of Type IIb autoimmune CSU), IL-31 (an itch mediator secreted by mast cells and basophils), and eosinophils (an indirect mediator of mast cell activation) at Week 12 relative to patients receiving the placebo (see Table 53).

Patients treated with 400 mg TID rilzabrutinib exhibited a 22.8% and 8.1% reduction from baseline median sMRGPRX2 and IL-31 levels, respectively while patients treated with the placebo experienced a 4.5% reduction in sMRGPRX2 levels and an increase of 15.4% in IL-31 levels.

Additionally, patients receiving 400 mg TID rilzabrutinib achieved a 46.7% and 38.7% reduction from baseline for IgG anti-TPO and IgG anti-FCFRI, respectively, while patients receiving the placebo achieved only a 7.0% decrease in IgG anti-TPO and a 14.2% increase in IgG anti-FCFRI. The reduction in IgG anti-FCεRI following inhibition of BTK signaling with rilzabrutinib may result in a reduction in mast-cell and basophil activation.

Patients receiving 400 mg TID rilzabrutinib also exhibited a 29.0% reduction in eosinophil levels, while patients receiving the placebo experienced a 3.6% increase in eosinophil levels.

Treatment with rilzabrutinib was not associated with a change in total serum IgE or IgG levels.

TABLE 53
Summary of Change in Biomarkers from Baseline to Week 12

| Median Percent | | | | |
|---|---|---|---|---|
| Change from | | Rilzabrutinib | Rilzabrutinib | Rilzabrutinib |
| Baseline | Placebo | 400 mg QPM | 400 mg BID | 400 mg TID |
| (Q1; Q3 range) | (N = 36) | (N = 37) | (N = 35) | (N = 35) |
| Total IgE (IU/mL) | 0 | 3.4 | 3.9 | 4.1 |
| | (−13.8; 25.3) | (−13.4; 22.2) | (−13.0; 34.1) | (−13.4; 53.7) |
| Total IgG (g/L) | 2.76 | −1.99 | 0.96 | 0.24 |
| | (−2.29; 6.360 | (−6.23; 5.89) | (−2.21; 5.45) | (−7.99; 4.61) |
| IgG anti-FcεRI | 14.2 | −20.4 | −39.0 | −38.7 |
| (arbitrary unit) | (−76.9; 202.4) | (−92.4; 90.2) | (−91.5; − 5.7) | (−91.8; 162.9) |
| IgG anti-TPO (IU) | −7.0 | −39.9 | −19.4 | −46.7 |
| | (−62.4; 53.0) | (−100.0; − 0.7) | (−71.5; 16.7) | (−69.0; − 7.4) |
| IL-31 (ng/mL) | 15.4 | −11.6 | −6.8 | −8.1 |
| | (−11.1; 31.3) | (−24.7; 17.9) | (−28.6; 9.9) | (−22.3; 15.0) |
| SMRGPRX2 | −4.5 | −31.1 | −29.3 | −22.8 |
| (ng/mL) | (−47.6; 59.0) | (−47.4; − 13.0) | (−44.9; 13.0) | (−57.7; − 6.4) |
| Eosinophils | 3.6 | 7.7 | −11.8 | −29.0 |
| (10⁹/L) | (−25.0; 45.5) | (−25.0; 33.3) | (−36.6; 8.7) | (−47.5; 0.0) |

### Safety

Rilzabrutinib was generally safe, with no evidence of new safety concerns or dose-related AEs (see Table 54). Consistent with this, vital signs and ECG results were similar across all groups.

Participants receiving 400 mg BID or TID rilzabrutinib experienced more TEAEs than patients receiving 400 mg QD rilzabrutinib or the placebo. The majority of TEAEs in patients receiving 400 mg TID and BID rilzabrutinib were reported as mild, with the most frequently occurring TEAEs being diarrhea, nausea, and headaches. Skin-related AEs were more frequent in participants receiving the placebo or 400 mg QD rilzabrutinib than in patients receiving 400 mg BID or TID rilzabrutinib.

Throughout the study, there were 3 treatment-emergent SAEs and no deaths. SAEs occurred at the same incidence rate across all rilzabrutinib dosing groups. There were no severe/serious infections or opportunistic infections. Additionally, there were no drug-induced liver injuries (including Hy's law). ALT increases>3×ULN, which meet the criteria for an AESI, were observed in 2 patients in the QPM cohort, 1 patient in the BID cohort, and 1 patient in the TID cohort. Of these patients, 3 recovered while continuing rilzabrutinib treatment. Notably, no TEAEs observed in clinical trials of other BTK inhibitors were observed in this study, further attesting to the safety of rilzabrutinib.

TABLE 54
Overview of Adverse Event Profile: Treatment-Emergent Adverse
Events in the Double-Blind Period (Safety Population)

| | | Rilzabrutinib | Rilzabrutinib | Rilzabrutinib |
|---|---|---|---|---|
| | Placebo | 400 mg QPM | 400 mg BID | 400 mg TID |
| n (%) | (N = 40) | (N = 38) | (N = 41) | (N = 41) |
| Participants with any TEAE | 23 (57.5) | 23 (60.5) | 30 (73.2) | 31 (75.6) |
| Participants with any severe TEAE | 1 (2.5) | 1 (2.6) | 1 (2.4) | 1 (2.4) |
| Participants with any treatment-emergent SAE | 1 (2.5) | 0 | 0 | 2 (4.9) |
| Participants with any TEAE leading to death | 0 | 0 | 0 | 0 |
| Participants with any TEAE leading to permanent | 0 | 0 | 1 (2.4) | 1 (2.4) |
| study intervention discontinuation | | | | |
| Participants with any treatment-emergent AESI | 2 (5.0) | 4 (10.5) | 4 (9.8) | 3 (7.3) |
| Participants with any TEAE related to IMP | 8 (20.0) | 9 (23.7) | 22 (53.7) | 22 (53.7) |

TEAE: Treatment-emergent adverse event, SAE: Serious adverse event, AESI: Adverse event of special interest
n (%) = number and percentage of participants with at least one TEAE.

### EMBODIMENTS

Non-limiting embodiments of the disclosure include:
1. A method of treating chronic spontaneous urticaria (CSU) in a human patient in need thereof, comprising administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.
2. The method of Embodiment 1, wherein the human patient has moderate-to-severe CSU.
3. The method of Embodiment 1 or 2, wherein the human patient has moderate CSU.
4. The method of Embodiment 1 or 2, wherein the human patient has severe CSU.
5. The method of any one of the preceding Embodiments, wherein the human patient has CSU refractory to H1 antihistamine (H1-AH) treatment.
6. The method of any one of Embodiments 1-5, wherein the human patient has Type I autoimmune CSU.
7. The method of any one of Embodiments 1-5, wherein the human patient has Type IIb autoimmune CSU.
8. The method of any one of the preceding Embodiments, wherein the human patient has angioedema.
9. The method of any one of the preceding Embodiments, wherein the human patient achieves a Urticaria Activity Score (UAS7) that is lower than baseline UAS7.
10. The method of any one of the preceding Embodiments, wherein the human patient achieves a UAS7 that is lower than baseline UAS7 within 52 weeks of initiating rilzabrutinib treatment.
11. The method of any one of the preceding Embodiments, wherein the human patient achieves a UAS7 that is lower than baseline UAS7 within 24 weeks of initiating rilzabrutinib treatment.
12. The method of any one of the preceding Embodiments, wherein the human patient achieves a UAS7 that is lower than baseline UAS7 within 12 weeks of initiating rilzabrutinib treatment.
13. The method of any one of the preceding Embodiments, wherein the human patient achieves a UAS7 that is lower than baseline UAS7 within 4 weeks of initiating rilzabrutinib treatment.
14. The method of any one of the preceding Embodiments, wherein the human patient achieves a UAS7≤6.
15. The method of any one of the preceding Embodiments, wherein the human patient achieves a UAS7≤6 within 12 weeks of initiating rilzabrutinib treatment.
16. The method of any one of the preceding Embodiments, wherein the human patient achieves a UAS7 of 0.
17. The method of any one of the preceding Embodiments, wherein the human patient achieves a UAS7 of 0 within 12 weeks of initiating rilzabrutinib treatment.
18. The method of any one of the preceding Embodiments, wherein the human patient achieves a UAS7 that is at least 10 points lower than baseline UAS7.
19. The method of any one of the preceding Embodiments, wherein the human patient achieves an Itch Severity Score (ISS7) that is lower than baseline ISS7.
20. The method of any one of the preceding Embodiments, wherein the human patient achieves an ISS7 that is lower than baseline ISS7 within 52 weeks of initiating rilzabrutinib treatment.
21. The method of any one of the preceding Embodiments, wherein the human patient achieves an ISS7 that is lower than baseline ISS7 within 24 weeks of initiating rilzabrutinib treatment.
22. The method of any one of the preceding Embodiments, wherein the human patient achieves an ISS7 that is lower than baseline ISS7 within 12 weeks of initiating rilzabrutinib treatment.
23. The method of any one of the preceding Embodiments, wherein the human patient achieves an ISS7 that is at least 5 points lower than baseline ISS7.
24. The method of any one of the preceding Embodiments, wherein the human patient achieves a Hives Severity Score (HSS7) that is lower than baseline HSS7.
25. The method of any one of the preceding Embodiments, wherein the human patient achieves a HSS7 that is lower than baseline HSS7 within 52 weeks of initiating rilzabrutinib treatment.
26. The method of any one of the preceding Embodiments, wherein the human patient achieves a HSS7 that is lower than baseline HSS7 within 24 weeks of initiating rilzabrutinib treatment.
27. The method of any one of the preceding Embodiments, wherein the human patient achieves a HSS7 that is lower than baseline HSS7 within 12 weeks of initiating rilzabrutinib treatment.
28. The method of any one of the preceding Embodiments, wherein the human patient achieves an Angioedema Activity Score (AAS7) that is lower than baseline AAS7.
29. The method of any one of the preceding Embodiments, wherein the human patient achieves an AAS7 that is lower than baseline AAS7 within 52 weeks of initiating rilzabrutinib treatment.
30. The method of any one of the preceding Embodiments, wherein the human patient achieves an AAS7 that is lower than baseline AAS7 within 24 weeks of initiating rilzabrutinib treatment.
31. The method of any one of the preceding Embodiments, wherein the human patient achieves an AAS7 that is lower than baseline AAS7 within 12 weeks of initiating rilzabrutinib treatment.
32. The method of any one of the preceding Embodiments, wherein the human patient achieves a Urticaria Control Test (UCT) score that is higher than baseline UCT score.
33. The method of any one of the preceding Embodiments, wherein the human patient achieves a UCT score that is higher than baseline UCT score within 52 weeks of initiating rilzabrutinib treatment.
34. The method of any one of the preceding Embodiments, wherein the human patient achieves a UCT score that is higher than baseline UCT score within 24 weeks of initiating rilzabrutinib treatment.
35. The method of any one of the preceding Embodiments, wherein the human patient achieves a UCT score that is higher than baseline UCT score within 12 weeks of initiating rilzabrutinib treatment.
36. The method of any one of the preceding Embodiments, wherein the human patient achieves a UCT score that is higher than baseline UCT score within 8 weeks of initiating rilzabrutinib treatment.
37. The method of any one of the preceding Embodiments, wherein the human patient achieves a UCT score that is higher than baseline UCT score within 4 weeks of initiating rilzabrutinib treatment.
38. The method of any one of the preceding Embodiments, wherein the human patient achieves a UCT score≥12.
39. The method of any one of the preceding Embodiments, wherein the human patient achieves a UCT score≥12 within 12 weeks of initiating rilzabrutinib treatment.
40. The method of any one of the preceding Embodiments, wherein the human patient achieves a UCT score≥12 within 8 weeks of initiating rilzabrutinib treatment.
41. The method of any one of the preceding Embodiments, wherein the human patient achieves a UCT score≥12 within 4 weeks of initiating rilzabrutinib treatment.
42. The method of any one of the preceding Embodiments, wherein the human patient achieves a change from baseline in health-related quality-of-life (HRQoL).
43. The method of any one of the preceding Embodiments, wherein the human patient achieves a change from baseline in HRQoL as measured by the Patient Global Impression of Severity (PGIS).
44. The method of any one of the preceding Embodiments, wherein the human patient achieves a change from baseline in HRQoL as measured by the PGIS within 12 weeks of initiating rilzabrutinib treatment.
45. The method of any one of the preceding Embodiments, wherein the human patient achieves a change from baseline in HRQoL as measured by the PGIS within 8 weeks of initiating rilzabrutinib treatment.
46. The method of any one of the preceding Embodiments, wherein the human patient achieves a change from baseline in HRQoL as measured by the PGIS within 4 weeks of initiating rilzabrutinib treatment.
47. The method of any one of the preceding Embodiments, wherein the human patient achieves a change from baseline in HRQoL as measured by the PGIS within 2 weeks of initiating rilzabrutinib treatment.
48. The method of any one of the preceding Embodiments, wherein the human patient achieves a change from baseline in HRQoL as measured by the PGIC.
49. The method of any one of the preceding Embodiments, wherein the human patient achieves a change from baseline in HRQoL as measured by the PGIC within 12 weeks of initiating rilzabrutinib treatment.
50. The method of any one of the preceding Embodiments, wherein the human patient achieves a change from baseline in HRQoL as measured by the PGIC within 8 weeks of initiating rilzabrutinib treatment.
51. The method of any one of the preceding Embodiments, wherein the human patient achieves a change from baseline in HRQoL as measured by the PGIC within 4 weeks of initiating rilzabrutinib treatment.
52. The method of any one of the preceding Embodiments, wherein the human patient achieves a change from baseline in HRQoL as measured by the PGIC within 2 weeks of initiating rilzabrutinib treatment.
53. The method of any one of the preceding Embodiments, wherein the human patient requires rescue therapy.
54. The method of Embodiment 53, wherein the total number of days for which the human patient requires rescue therapy is lower than the total number of days for which the human patient required rescue therapy during a baseline time period of the same length.
55. The method of any one of the preceding claims, wherein the human patient has a history of taking omalizumab prior to the start of the treatment period.
56. The method of any one of the preceding Embodiments, wherein the human patient has had an incomplete response to omalizumab.
57. The method of any one of Embodiments 1-54, wherein the human patient is omalizumab naïve.
58. The method of any one of the preceding Embodiments, wherein the human patient has a positive basophil histamine release assay (BHRA) status.
59. The method of any one of the preceding Embodiments, wherein the human patient receives concomitant treatment with an H1-AH.
60. The method of any one of the preceding Embodiments, wherein the human patient has previously been identified as having increased sMRGPRX2 serum levels.
61. The method of any one of the preceding Embodiments, wherein the human patient has previously been identified as having increased mast cell MRGPRX2 expression.
62. The method of any one of the preceding Embodiments, wherein the human patient achieves a reduction from baseline in sMRGPRX2 serum levels.
63. The method of any one of the preceding Embodiments, wherein the human patient achieves a reduction from baseline in sMRGPRX2 serum levels within 12 weeks of initiating rilzabrutinib treatment.
64. The method of any one of the preceding Embodiments, wherein the human patient achieves a reduction from baseline in sMRGPRX2 serum levels within 8 weeks of initiating rilzabrutinib treatment.
65. The method of any one of the preceding Embodiments, wherein the human patient achieves a reduction from baseline in sMRGPRX2 serum levels within 4 weeks of initiating rilzabrutinib treatment.
66. The method of any one of the preceding Embodiments, wherein the human patient achieves a reduction from baseline in sMRGPRX2 serum levels within 2 weeks of initiating rilzabrutinib treatment.
67. The method of any one of the preceding Embodiments, wherein the human patient has previously been identified as having increased IL-31 serum levels.
68. The method of any one of the preceding Embodiments, wherein the human patient has previously been identified as having increased IL-31 serum levels as a result of increased mast cell or basophil activation.
69. The method of any one of the preceding Embodiments, wherein the human patient achieves a reduction from baseline in IL-31 serum levels.
70. The method of any one of the preceding Embodiments, wherein the human patient achieves a reduction from baseline in IL-31 serum levels within 12 weeks of initiating rilzabrutinib treatment.
71. The method of any one of the preceding Embodiments, wherein the human patient achieves a reduction from baseline in IL-31 serum levels within 8 weeks of initiating rilzabrutinib treatment.
72. The method of any one of the preceding Embodiments, wherein the human patient achieves a reduction from baseline in IL-31 serum levels within 4 weeks of initiating rilzabrutinib treatment.
73. The method of any one of the preceding Embodiments, wherein the human patient achieves a reduction from baseline in IL-31 serum levels within 2 weeks of initiating rilzabrutinib treatment.
74. The method of any one of the preceding Embodiments, wherein the human patient has previously been identified as having increased IgG anti-TPO serum levels.
75. The method of any one of the preceding Embodiments, wherein the human patient achieves a reduction from baseline in IgG anti-TPO serum levels.
76. The method of any one of the preceding Embodiments, wherein the human patient achieves a reduction from baseline in IgG anti-TPO serum levels within 12 weeks of initiating rilzabrutinib treatment.
77. The method of any one of the preceding Embodiments, wherein the human patient achieves a reduction from baseline in IgG anti-TPO serum levels within 8 weeks of initiating rilzabrutinib treatment.
78. The method of any one of the preceding Embodiments, wherein the human patient achieves a reduction from baseline in IgG anti-TPO serum levels within 4 weeks of initiating rilzabrutinib treatment.
79. The method of any one of the preceding Embodiments, wherein the human patient achieves a reduction from baseline in IgG anti-TPO serum levels within 2 weeks of initiating rilzabrutinib treatment.
80. The method of any one of the preceding Embodiments, wherein the human patient has previously been identified as having increased IgG anti-FCεRI serum levels.
81. The method of any one of the preceding Embodiments, wherein the human patient has previously been identified as having increased mast cell or basophil activation as a result of increased IgG anti-FCεRI levels serum levels.
82. The method of any one of the preceding Embodiments, wherein the human patient achieves a reduction from baseline in IgG anti-FCεRI serum levels.
83. The method of any one of the preceding Embodiments, wherein the human patient achieves a reduction from baseline in IgG anti-FCεRI serum levels within 12 weeks of initiating rilzabrutinib treatment.
84. The method of any one of the preceding Embodiments, wherein the human patient achieves a reduction from baseline in IgG anti-FCεRI serum levels within 8 weeks of initiating rilzabrutinib treatment.
85. The method of any one of the preceding Embodiments, wherein the human patient achieves a reduction from baseline in IgG anti-FCεRI serum levels within 4 weeks of initiating rilzabrutinib treatment.
86. The method of any one of the preceding Embodiments, wherein the human patient achieves a reduction from baseline in IgG anti-FCεRI serum levels within 2 weeks of initiating rilzabrutinib treatment.
87. The method of any one of the preceding Embodiments, wherein the human patient has previously been identified as having increased eosinophil levels.
88. The method of any one of the preceding Embodiments, wherein the human patient has previously been identified as having increased mast cell activation as a result of increased eosinophil levels.
89. The method of any one of the preceding Embodiments, wherein the human patient achieves a reduction from baseline in eosinophil levels.
90. The method of any one of the preceding Embodiments, wherein the human patient achieves a reduction from baseline in eosinophil levels within 12 weeks of initiating rilzabrutinib treatment.
91. The method of any one of the preceding Embodiments, wherein the human patient achieves a reduction from baseline in eosinophil levels within 8 weeks of initiating rilzabrutinib treatment.
92. The method of any one of the preceding Embodiments, wherein the human patient achieves a reduction from baseline in eosinophil levels within 4 weeks of initiating rilzabrutinib treatment.
93. The method of any one of the preceding Embodiments, wherein the human patient achieves a reduction from baseline in eosinophil levels within 2 weeks of initiating rilzabrutinib treatment.
94. The method of any one of the preceding Embodiments, wherein the human patient does not achieve a reduction in total serum IgG or IgE levels.
95. A method for treating chronic spontaneous urticaria (CSU) in a human patient in need thereof, comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has CSU refractory to H1 antihistamines (H1-AH) treatment.
96. The method of any one of the preceding Embodiments, wherein the treatment period is at least 12 weeks.
97. The method of any one of the preceding Embodiments, wherein the treatment period is at least 52 weeks.
98. The method of any one of the preceding Embodiments, wherein the at least one compound consists of at least one compound chosen from the (E) isomer of rilzabrutinib and pharmaceutically acceptable salts thereof.
99. The method of any one of Embodiments 1-97, wherein the at least one compound consists of at least one compound chosen from the (Z) isomer of rilzabrutinib and pharmaceutically acceptable salts thereof.
100. The method of any one of Embodiments 1-97, wherein the at least one compound consists of a mixture of (E) and (Z) isomers of rilzabrutinib or a pharmaceutically acceptable salt of the foregoing.
101. The method of any one of the preceding Embodiments, comprising administering to the human patient rilzabrutinib once, twice, or thrice a day.
102. The method of any one of the preceding Embodiments, comprising administering to the human patient 400 mg of rilzabrutinib once, twice, or thrice a day.
103. The method of any one of the preceding Embodiments, comprising administering to the human patient 400 mg of rilzabrutinib once a day.
104. The method of any one of Embodiments 1-102, comprising administering to the human patient 400 mg of rilzabrutinib twice a day.
105. The method of any one of Embodiments 1-102, comprising administering to the human patient 400 mg of rilzabrutinib thrice a day.
106. The method of any one of Embodiments 1-98 and 101-105 wherein the at least one compound is the (E) isomer of rilzabrutinib.
107. The method of any one of Embodiments 1-97, 99, and 101-105, wherein the at least one compound is the (Z) isomer of rilzabrutinib.
108. The method of any one of Embodiments 1-97 and 100-105, wherein the at least one compound consists of a mixture of (E) and (Z) isomers of rilzabrutinib.
109. The method of any one of the preceding Embodiments, wherein the at least one compound is orally administered to the human patient.
110. The method of any one of the preceding Embodiments, wherein the at least one compound is administered to the human patient in the form of at least one tablet.
111. The method of any one of the preceding Embodiments, wherein the at least one compound is administered with water.
112. The method of any one of Embodiments 1-100 and 109-111, wherein the at least one compound is rilzabrutinib.
113. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof.
114. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof.
115. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof.
116. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for the manufacture of a medicament for treating chronic spontaneous urticaria (CSU) in a human patient in need thereof.
117. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof, wherein the human patient in need thereof has CSU refractory to H1 antihistamines (H1-AH) treatment.
118. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof, wherein the human patient in need thereof has CSU refractory to H1 antihistamines (H1-AH) treatment.
119. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof, wherein the human patient in need thereof has CSU refractory to H1 antihistamines (H1-AH) treatment.
120. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, in the manufacture of a medicament for treating chronic spontaneous urticaria (CSU) in a human patient in need thereof, wherein the human patient in need thereof has CSU refractory to H1 antihistamines (H1-AH) treatment.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed is:

1. A method of treating chronic spontaneous urticaria (CSU) in a human patient in need thereof, comprising administering to the human patient in need thereof a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof.

2. The method of claim 1, wherein the human patient has moderate-to-severe CSU.

3. The method of claim 1 or 2, wherein the human patient has moderate CSU.

4. The method of claim 1 or 2, wherein the human patient has severe CSU.

5. The method of any one of the preceding claims, wherein the human patient has CSU refractory to H1antihistamine (H1-AH) treatment.

6. The method of any one of claims 1-5, wherein the human patient has Type I autoimmune CSU.

7. The method of any one of claims 1-5, wherein the human patient has Type IIb autoimmune CSU.

8. The method of any one of the preceding claims, wherein the human patient has angioedema.

9. The method of any one of the preceding claims, wherein the human patient achieves a Urticaria Activity Score (UAS7) that is lower than baseline UAS7.

10. The method of any one of the preceding claims, wherein the human patient achieves a UAS7 that is lower than baseline UAS7 within 52 weeks of initiating rilzabrutinib treatment.

11. The method of any one of the preceding claims, wherein the human patient achieves a UAS7 that is lower than baseline UAS7 within 24 weeks of initiating rilzabrutinib treatment.

12. The method of any one of the preceding claims, wherein the human patient achieves a UAS7 that is lower than baseline UAS7 within 12 weeks of initiating rilzabrutinib treatment.

13. The method of any one of the preceding claims, wherein the human patient achieves a UAS7 that is lower than baseline UAS7 within 4 weeks of initiating rilzabrutinib treatment.

14. The method of any one of the preceding claims, wherein the human patient achieves a UAS7≤6.

15. The method of any one of the preceding claims, wherein the human patient achieves a UAS7≤6 within 12 weeks of initiating rilzabrutinib treatment.

16. The method of any one of the preceding claims, wherein the human patient achieves a UAS7 of 0.

17. The method of any one of the preceding claims, wherein the human patient achieves a UAS7 of 0 within 12 weeks of initiating rilzabrutinib treatment.

18. The method of any one of the preceding claims, wherein the human patient achieves a UAS7 that is at least 10 points lower than baseline UAS7.

19. The method of any one of the preceding claims, wherein the human patient achieves an Itch Severity Score (ISS7) that is lower than baseline ISS7.

20. The method of any one of the preceding claims, wherein the human patient achieves an ISS7 that is lower than baseline ISS7 within 52 weeks of initiating rilzabrutinib treatment.

21. The method of any one of the preceding claims, wherein the human patient achieves an ISS7 that is lower than baseline ISS7 within 24 weeks of initiating rilzabrutinib treatment.

22. The method of any one of the preceding claims, wherein the human patient achieves an ISS7 that is lower than baseline ISS7 within 12 weeks of initiating rilzabrutinib treatment.

23. The method of any one of the preceding claims, wherein the human patient achieves an ISS7 that is at least 5 points lower than baseline ISS7.

24. The method of any one of the preceding claims, wherein the human patient achieves a Hives Severity Score (HSS7) that is lower than baseline HSS7.

25. The method of any one of the preceding claims, wherein the human patient achieves a HSS7 that is lower than baseline HSS7 within 52 weeks of initiating rilzabrutinib treatment.

26. The method of any one of the preceding claims, wherein the human patient achieves a HSS7 that is lower than baseline HSS7 within 24 weeks of initiating rilzabrutinib treatment.

27. The method of any one of the preceding claims, wherein the human patient achieves a HSS7 that is lower than baseline HSS7 within 12 weeks of initiating rilzabrutinib treatment.

28. The method of any one of the preceding claims, wherein the human patient achieves an Angioedema Activity Score (AAS7) that is lower than baseline AAS7.

29. The method of any one of the preceding claims, wherein the human patient achieves an AAS7 that is lower than baseline AAS7 within 52 weeks of initiating rilzabrutinib treatment.

30. The method of any one of the preceding claims, wherein the human patient achieves an AAS7 that is lower than baseline AAS7 within 24 weeks of initiating rilzabrutinib treatment.

31. The method of any one of the preceding claims, wherein the human patient achieves an AAS7 that is lower than baseline AAS7 within 12 weeks of initiating rilzabrutinib treatment.

32. The method of any one of the preceding claims, wherein the human patient achieves a Urticaria Control Test (UCT) score that is higher than baseline UCT score.

33. The method of any one of the preceding claims, wherein the human patient achieves a UCT score that is higher than baseline UCT score within 52 weeks of initiating rilzabrutinib treatment.

34. The method of any one of the preceding claims, wherein the human patient achieves a UCT score that is higher than baseline UCT score within 24 weeks of initiating rilzabrutinib treatment.

35. The method of any one of the preceding claims, wherein the human patient achieves a UCT score that is higher than baseline UCT score within 12 weeks of initiating rilzabrutinib treatment.

36. The method of any one of the preceding claims, wherein the human patient achieves a UCT score that is higher than baseline UCT score within 8 weeks of initiating rilzabrutinib treatment.

37. The method of any one of the preceding claims, wherein the human patient achieves a UCT score that is higher than baseline UCT score within 4 weeks of initiating rilzabrutinib treatment.

38. The method of any one of the preceding claims, wherein the human patient achieves a UCT score 12.

39. The method of any one of the preceding claims, wherein the human patient achieves a UCT score 12 within 12 weeks of initiating rilzabrutinib treatment.

40. The method of any one of the preceding claims, wherein the human patient achieves a UCT score 12 within 8 weeks of initiating rilzabrutinib treatment.

41. The method of any one of the preceding claims, wherein the human patient achieves a UCT score 12 within 4 weeks of initiating rilzabrutinib treatment.

42. The method of any one of the preceding claims, wherein the human patient achieves a change from baseline in health-related quality-of-life (HRQoL).

43. The method of any one of the preceding claims, wherein the human patient achieves a change from baseline in HRQoL as measured by the Patient Global Impression of Severity (PGIS).

44. The method of any one of the preceding claims, wherein the human patient achieves a change from baseline in HRQoL as measured by the PGIS within 12 weeks of initiating rilzabrutinib treatment.

45. The method of any one of the preceding claims, wherein the human patient achieves a change from baseline in HRQoL as measured by the PGIS within 8 weeks of initiating rilzabrutinib treatment.

46. The method of any one of the preceding claims, wherein the human patient achieves a change from baseline in HRQoL as measured by the PGIS within 4 weeks of initiating rilzabrutinib treatment.

47. The method of any one of the preceding claims, wherein the human patient achieves a change from baseline in HRQoL as measured by the PGIS within 2 weeks of initiating rilzabrutinib treatment.

48. The method of any one of the preceding claims, wherein the human patient achieves a change from baseline in HRQoL as measured by the PGIC.

49. The method of any one of the preceding claims, wherein the human patient achieves a change from baseline in HRQoL as measured by the PGIC within 12 weeks of initiating rilzabrutinib treatment.

50. The method of any one of the preceding claims, wherein the human patient achieves a change from baseline in HRQoL as measured by the PGIC within 8 weeks of initiating rilzabrutinib treatment.

51. The method of any one of the preceding claims, wherein the human patient achieves a change from baseline in HRQoL as measured by the PGIC within 4 weeks of initiating rilzabrutinib treatment.

52. The method of any one of the preceding claims, wherein the human patient achieves a change from baseline in HRQoL as measured by the PGIC within 2 weeks of initiating rilzabrutinib treatment.

53. The method of any one of the preceding claims, wherein the human patient requires rescue therapy.

54. The method of claim 53, wherein the total number of days for which the human patient requires rescue therapy is lower than the total number of days for which the human patient required rescue therapy during a baseline time period of the same length.

55. The method of any one of the preceding claims, wherein the human patient has a history of taking omalizumab prior to the start of the treatment period.

56. The method of any one of the preceding claims, wherein the human patient has had an incomplete response to omalizumab.

57. The method of any one of claims 1-55, wherein the human patient is omalizumab naïve.

58. The method of any one of the preceding claims, wherein the human patient has a positive basophil histamine release assay (BHRA) status.

59. The method of any one of the preceding claims, wherein the human patient receives concomitant treatment with an H1-AH.

60. A method of treating chronic spontaneous urticaria (CSU) in a human patient in need thereof, comprising administering to the human patient a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, wherein the human patient in need thereof has CSU refractory to H1 antihistamines (H1-AH) treatment.

61. The method of any one of the preceding claims, wherein the treatment period is at least 12 weeks.

62. The method of any one of the preceding claims, wherein the treatment period is at least 52 weeks.

63. The method of any one of the preceding claims, wherein the at least one compound consists of at least one compound chosen from the (E) isomer of rilzabrutinib and pharmaceutically acceptable salts thereof.

64. The method of any one of claims 1-62, wherein the at least one compound consists of at least one compound chosen from the (Z) isomer of rilzabrutinib and pharmaceutically acceptable salts thereof.

65. The method of any one of claims 1-62, wherein the at least one compound consists of a mixture of (E) and (Z) isomers of rilzabrutinib or a pharmaceutically acceptable salt of the foregoing.

66. The method of any one of the preceding claims, comprising administering to the human patient rilzabrutinib once, twice, or thrice a day.

67. The method of any one of the preceding claims, comprising administering to the human patient 400 mg of rilzabrutinib once, twice, or thrice a day.

68. The method of any one of the preceding claims, comprising administering to the human patient 400 mg of rilzabrutinib once a day.

69. The method of any one of claims 1-67, comprising administering to the human patient 400 mg of rilzabrutinib twice a day.

70. The method of any one of claims 1-67, comprising administering to the human patient 400 mg of rilzabrutinib thrice a day.

71. The method of any one of claims 1-63 and 66-70 wherein the at least one compound is the (E) isomer of rilzabrutinib.

72. The method of any one of claims 1-62, 64, and 66-70, wherein the at least one compound is the (Z) isomer of rilzabrutinib.

73. The method of any one of claims 1-62 and 65-70, wherein the at least one compound consists of a mixture of (E) and (Z) isomers of rilzabrutinib.

74. The method of any one of the preceding claims, wherein the at least one compound is orally administered to the human patient.

75. The method of any one of the preceding claims, wherein the at least one compound is administered to the human patient in the form of at least one tablet.

76. The method of any one of the preceding claims, wherein the at least one compound is administered with water.

77. The method of any one of claims 1-65 and 74-76, wherein the at least one compound is rilzabrutinib.

78. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof.

79. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof.

80. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof.

81. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for the manufacture of a medicament for treating chronic spontaneous urticaria (CSU) in a human patient in need thereof.

82. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in treating chronic spontaneous urticaria (CSU) in a human patient in need thereof, wherein the human patient in need thereof has CSU refractory to H1 antihistamines (H1-AH) treatment.

83. A therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for use in the manufacture of a medicament for treating chronic spontaneous urticaria (CSU) in a human patient in need thereof, wherein the human patient in need thereof has CSU refractory to H1 antihistamines (H1-AH) treatment.

84. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, for treating chronic spontaneous urticaria (CSU) in a human patient in need thereof, wherein the human patient in need thereof has CSU refractory to H1 antihistamines (H1-AH) treatment.

85. Use of a therapeutically effective amount of at least one compound chosen from (R)-2-[3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (rilzabrutinib) and pharmaceutically acceptable salts thereof, in the manufacture of a medicament for treating chronic spontaneous urticaria (CSU) in a human patient in need thereof, wherein the human patient in need thereof has CSU refractory to H1 antihistamines (H1-AH) treatment.
